(19) United States
(12) Patent Application Publication
(10) Pub. No.: US 2025/0326753 A1
(43) Pub. Date: Oct. 23, 2025

(54) DUAL CXCR4-BTK INHIBITORS

(71) Applicant: X4 Pharmaceuticals, Inc., Boston, MA (US)

(72) Inventors: Art Taveras, Boston, MA (US); Angelina Roberta Sekirnik, Logatec (SI); Chi Nguyen, Vienna (AT); Elyse Marie Josee Bourque, Quebec (CA)

(21) Appl. No.: 18/850,818

(22) PCT Filed: Mar. 27, 2023
(86) PCT No.: PCT/US2023/016451

§ 371 Date: Sep. 25, 2024

Related U.S. Application Data
(60) Provisional application No. 63/323,996, filed on Mar. 25, 2022.

(51) Int. Cl. C07D 487/04; A61K 31/506; A61K 31/519; A61K 47/55; A61P 35/00; C07D 401/14

(52) U.S. Cl. CPC C07D 487/04; A61K 31/506; A61K 31/519; A61K 47/55; A61P 35/00; C07D 401/14

(57) ABSTRACT

The present invention relates to compounds and methods useful for dual inhibition of C-X-C receptor type 4 (CXCR4) and Bruton's tyrosine kinase (BTK). The invention also provides pharmaceutically acceptable compositions comprising compounds of the present invention and methods of using said compositions in the treatment of various disorders.

### CROSS-REFERENCE TO RELATED APPLICATIONS

This application claims the benefit of U.S. Provisional Patent Application No. 63/323,996, filed Mar. 25, 2022; the entirety of which is hereby incorporated by reference.

### TECHNICAL FIELD OF THE INVENTION

The present invention relates to compounds and methods useful for dual inhibition of C-X-C receptor type 4 (CXCR4) and Bruton's tyrosine kinase (BTK). The invention also provides pharmaceutically acceptable compositions comprising compounds of the present invention and methods of using said compositions in the treatment of various disorders.

### BACKGROUND OF THE INVENTION

C-X-C chemokine receptor type 4 (CXCR4), also known as fusin or cluster of differentiation 184 (CD184), is a seven transmembrane G-protein coupled receptor (GPCR) belonging to Class I GPCR or rhodopsin-like GPCR family. Under normal physiological conditions, CXCR4 carries out multiple roles and is principally expressed in the hematopoietic and immune systems. CXCR4 was initially discovered as one of the co-receptors involved in human immunodeficiency virus (HIV) cell entry. Subsequent studies showed that it is expressed in many tissues, including brain, thymus, lymphatic tissues, spleen, stomach, and small intestine, and also specific cell types such as hematopoietic stem cells (HSC), mature lymphocytes, and fibroblasts. CXCL12, previously designated SDF-1 a, is the only known ligand for CXCR4. CXCR4 mediates migration of stem cells during embryonic development as well as in response to injury and inflammation. Multiple roles have been demonstrated for CXCR4 in human diseases such as cellular proliferative disorders, Alzheimer's disease, HIV, rheumatoid arthritis, pulmonary fibrosis, and others. For example, expression of CXCR4 and CXCL12 have been noted in several tumor types. CXCL12 is expressed by cancer-associated fibroblast (CAFs) and is often present at high levels in the tumor microenvironment (TME). In clinical studies of a wide range of tumor types, including breast, ovarian, renal, lung, and melanoma, expression of CXCR4/CXCL12 has been associated with a poor prognosis and with an increased risk of metastasis to lymph nodes, lung, liver, and brain, which are sites of CXCL12 expression. CXCR4 is frequently expressed on melanoma cells, particularly the CD133+ population that is considered to represent melanoma stem cells; in vitro experiments and murine models have demonstrated that CXCL12 is chemotactic for such cells.

Furthermore, there is now evidence implicating the CXCL12/CXCR4 axis in contributing to the loss or lack of tumor responsiveness to angiogenesis inhibitors (also referred to as “angiogenic escape”). In animal cancer models, interference with CXCR4 function has been demonstrated to alter the TME and sensitize the tumor to immune attack by multiple mechanisms such as elimination of tumor re-vascularization and increasing the ratio of CD8+ T cells to Treg cells. These effects result in significantly decreased tumor burden and increased overall survival in xenograft, syngeneic, and transgenic cancer models. See Vanharanta et al. (2013) Nat Med 19: 50-56; Gale and McColl (1999) BioEssays 21: 17-28; Highfill et al. (2014) Sci Transl Med 6: ra67; Facciabene et al. (2011) Nature 475: 226-230.

Bruton's tyrosine kinase (BTK) belongs to the Tec tyrosine kinase family (Vetrie et al., Nature 361:226-233, 1993; Bradshaw, Cell Signal. 22: 1175-84, 2010). BTK is primarily expressed in most hematopoietic cells such as B cells, mast cells and macrophages (Smith et al., J. Immunol. 152: 557-565, 1994) and is localized in bone marrow, spleen and lymph node tissue. BTK plays an important role in B-cell receptor (BCR) and FcR signaling pathways, which are involved in B-cell development and differentiation (Khan, Immunol. Res. 23: 147, 2001). BTK is activated by upstream Src-family kinases. Once activated, BTK, in turn, phosphorylates PLC-gamma, leading to effects on B-cell function and survival (Humphries et al., J. Biol. Chem. 279: 37651, 2004). These signaling pathways must be precisely regulated. Mutations in the gene encoding BTK cause an inherited B-cell specific immunodeficiency disease in humans, known as X-linked agammaglobulinemia (XLA) (Conley et al., Annu. Rev. Immunol. 27: 199-227, 2009). Aberrant BCR-mediated signaling may result in dysregulated B-cell activation leading to a number of autoimmune and inflammatory diseases. Preclinical studies show that BTK deficient mice are resistant to developing collagen-induced arthritis. Moreover, clinical studies of Rituxan, a CD20 antibody to deplete mature B-cells, reveal the key role of B-cells in a number of inflammatory diseases such as rheumatoid arthritis, systemic lupus erythematosus and multiple sclerosis (Gurcan et al., Int. Immunopharmacol. 9: 10-25, 2009). Therefore, BTK inhibitors can be used to treat autoimmune and/or inflammatory diseases.

Inhibition of BTK has been shown to affect cancer development (B cell malignancies) and cell viability, and improve autoimmune diseases (e.g., rheumatoid arthritis, multiple sclerosis, and lupus). Inhibition of BTK has also been reported via alternative strategies, such as through degradation of BTK (Alexandru D. et al., Biochemistry 2018, 57, 26, 3564-3575; Adelajda Z. et al., PNAS 2018 115 (31); Dennis D., et al., Blood, 2019, 133: 952-961; Yonghui S. et al., Cell Research, 2018, 28, 779-781; Yonghui S. et al., Leukemia, 2019.

These data underscore the significant, unmet need for improved treatments for the many diseases and conditions mediated by aberrant or undesired expression of CXCR4 and BTK, for example in cellular proliferative disorders. The present invention addresses this need and provides certain other related advantages.

### SUMMARY OF THE INVENTION

It has now been found that compounds of the present invention, and pharmaceutically acceptable compositions thereof, are effective as dual CXCR4-BTK inhibitors. In certain embodiments, the invention provides compounds of the formulae presented herein.

Compounds of the present invention, and pharmaceutically acceptable compositions thereof, are useful for treating a variety of diseases, disorders or conditions associated with CXCR4 and BTK. Such diseases, disorders, or conditions include cellular proliferative disorders (e.g., cancer) such as those described herein.

In some embodiments, compounds provided by this invention are useful for the study of simultaneous inhibition of CXCR4 and BTK in biological and pathological phenomena, the study of cellular proliferative disorders, and the comparative evaluation of new dual CXCR4-BTK inhibitors or other regulators of cellular proliferation in vitro or in vivo.

### BRIEF DESCRIPTION OF THE FIGURES

FIG. 1A provides a visualization of the data of the table of kinase inhibition in Example 25 for compound I-10. FIG. 1B shows similar visualization for kinase inhibition by ARQ-531. The diameter of dots reflects the percent inhibition of 336 tested wild-type kinases. In comparison with the visualization for ARQ-531, the inhibitory effects of compound I-10 are shown to be surprisingly and unexpectedly precise and directed. The selectivity of compounds of the present invention, such as I-10, helps mitigate the risk of significant off-target effects that may cause adverse effects in patients.

### DETAILED DESCRIPTION OF CERTAIN EMBODIMENTS

### 1. General Description of Certain Embodiments of the Invention

In one aspect, the present invention provides a compound of formula I:

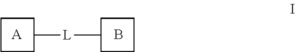

or a pharmaceutically acceptable salt thereof, wherein [symbol] is a small molecule BTK inhibitor; [symbol] is a small molecule or peptide CXCR4 inhibitor, or a CXCR4 antibody; and -L- is a covalent bond or a bivalent linker, and wherein each of [symbol] , [symbol] and -L- is as defined below and described in embodiments herein, both singly and in combination.

In some embodiments, the present invention provides a pharmaceutical composition comprising a compound of formula I and a pharmaceutically acceptable carrier, adjuvant, or diluent.

In some embodiments, the present invention provides a method of treating a disease, disorder, or condition associated with CXCR4 and/or BTK, comprising administering to a patient in need thereof a compound of formula I, or a pharmaceutically acceptable salt, solvate, or hydrate thereof. In some embodiments, the disease, disorder, or condition is treated by dual inhibition of CXCR4 and BTK.

The present invention also provides methods for synthesizing compounds of formula I.

### 2. Compounds and Definitions

Compounds of the present invention include those described generally herein, and are further illustrated by the classes, subclasses, and species disclosed herein. As used herein, the following definitions shall apply unless otherwise indicated. For purposes of this invention, the chemical elements are identified in accordance with the Periodic Table of the Elements, CAS version, Handbook of Chemistry and Physics, 75^{th} Ed. Additionally, general principles of organic chemistry are described in “Organic Chemistry,” Thomas Sorrell, University Science Books, Sausalito: 1999, and “March's Advanced Organic Chemistry,” 5^{th} Ed., Ed.: Smith, M. B. and March, J., John Wiley & Sons, New York: 2001, the entire contents of which are hereby incorporated by reference.

The term “aliphatic” or “aliphatic group,” as used herein, means a straight-chain (i.e., unbranched) or branched, substituted or unsubstituted hydrocarbon chain that is completely saturated or that contains one or more units of unsaturation, or a monocyclic hydrocarbon or bicyclic hydrocarbon that is completely saturated or that contains one or more units of unsaturation, but which is not aromatic (also referred to herein as “carbocycle,” “cycloaliphatic” or “cycloalkyl”), that has a single point of attachment to the rest of the molecule. Unless otherwise specified, aliphatic groups contain 1-6 aliphatic carbon atoms. In some embodiments, aliphatic groups contain 1-5 aliphatic carbon atoms. In other embodiments, aliphatic groups contain 1-4 aliphatic carbon atoms. In still other embodiments, aliphatic groups contain 1-3 aliphatic carbon atoms, and in yet other embodiments, aliphatic groups contain 1-2 aliphatic carbon atoms. In some embodiments, “cycloaliphatic” (or “carbocycle” or “cycloalkyl”) refers to a monocyclic C₃-C₆ hydrocarbon that is completely saturated or that contains one or more units of unsaturation, but which is not aromatic, that has a single point of attachment to the rest of the molecule. Suitable aliphatic groups include, but are not limited to, linear or branched, substituted or unsubstituted alkyl, alkenyl, alkynyl groups and hybrids thereof such as (cycloalkyl)alkyl, (cycloalkenyl)alkyl or (cycloalkyl)alkenyl.

As used herein, the term “bicyclic ring” or “bicyclic ring system” refers to any bicyclic ring system, i.e., carbocyclic or heterocyclic, saturated or having one or more units of unsaturation, having one or more atoms in common between the two rings of the ring system. Thus, the term includes any permissible ring fusion, such as ortho-fused or spirocyclic. As used herein, the term “heterobicyclic” is a subset of “bicyclic” that requires that one or more heteroatoms are present in one or both rings of the bicycle. Such heteroatoms may be present at ring junctions and are optionally substituted, and may be selected from nitrogen (including N-oxides), oxygen, sulfur (including oxidized forms such as sulfones and sulfonates), phosphorus (including oxidized forms such as phosphates), boron, etc. In some embodiments, a bicyclic group has 7-12 ring members and 0-4 heteroatoms independently selected from nitrogen, oxygen, or sulfur. As used herein, the term “bridged bicyclic” refers to any bicyclic ring system, i.e. carbocyclic or heterocyclic, saturated or partially unsaturated, having at least one bridge. As defined by IUPAC, a “bridge” is an unbranched chain of atoms or an atom or a valence bond connecting two bridgeheads, where a “bridgehead” is any skeletal atom of the ring system which is bonded to three or more skeletal atoms (excluding hydrogen). In some embodiments, a bridged bicyclic group has 7-12 ring members and 0-4 heteroatoms independently selected from nitrogen, oxygen, or sulfur. Such bridged bicyclic groups are well known in the art and include those groups set forth below where each group is attached to the rest of the molecule at any substitutable carbon or nitrogen atom. Unless otherwise specified, a bridged bicyclic group is optionally substituted with one or more substituents as set forth for aliphatic groups. Additionally or alternatively, any substitutable nitrogen of a bridged bicyclic group is optionally substituted. Exemplary bicyclic rings include:

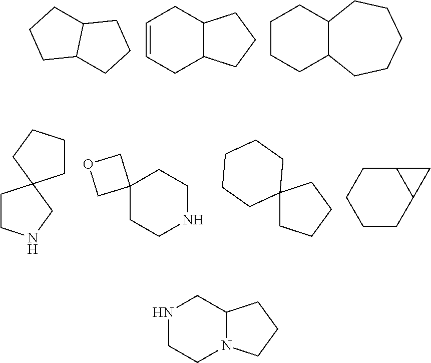

Exemplary bridged bicyclics include:

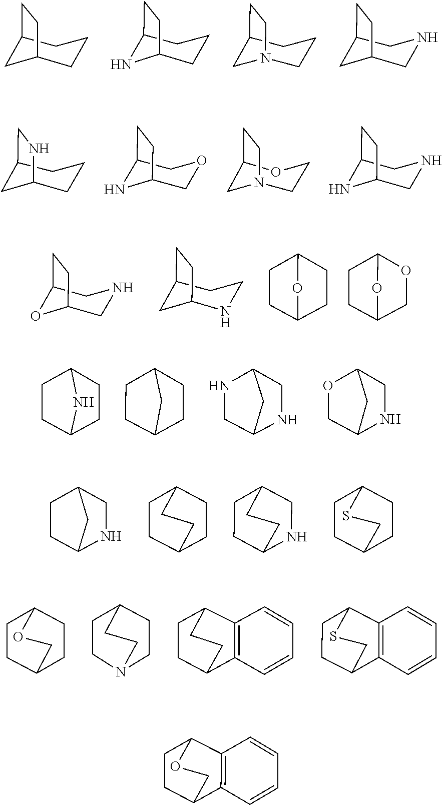

The term “lower alkyl” refers to a C₁₋₄ straight or branched alkyl group. Exemplary lower alkyl groups are methyl, ethyl, propyl, isopropyl, butyl, isobutyl, and tert-butyl.

The term “lower haloalkyl” refers to a C₁₋₄ straight or branched alkyl group that is substituted with one or more halogen atoms.

The term “heteroatom” means one or more of oxygen, sulfur, nitrogen, phosphorus, or silicon (including, any oxidized form of nitrogen, sulfur, phosphorus, or silicon; the quaternized form of any basic nitrogen or; a substitutable nitrogen of a heterocyclic ring, for example N (as in 3,4-dihydro-2H-pyrrolyl), NH (as in pyrrolidinyl) or NR⁻ (as in N-substituted pyrrolidinyl)).

The term “unsaturated,” as used herein, means that a moiety has one or more units of unsaturation.

As used herein, the term “bivalent C₁₋₈ (or C₁₋₆) saturated or unsaturated, straight or branched, hydrocarbon chain,” and similar terms, refer to bivalent alkylene, alkenylene, and alkynylene chains that are straight or branched as defined herein and contain the specified number of carbon atoms.

The term “alkylene” refers to a bivalent alkyl group. An “alkylene chain” is a polymethylene group, i.e., —(CH₂)ₙ—, wherein n is a positive integer, preferably from 1 to 6, from 1 to 4, from 1 to 3, from 1 to 2, or from 2 to 3. A substituted alkylene chain is a polymethylene group in which one or more methylene hydrogen atoms are replaced with a substituent. Suitable substituents include those described below for a substituted aliphatic group.

The term “alkenylene” refers to a bivalent alkenyl group. A substituted alkenylene chain is a polymethylene group containing at least one double bond in which one or more hydrogen atoms are replaced with a substituent. Suitable substituents include those described below for a substituted aliphatic group.

The term “halogen” means F, Cl, Br, or I.

The term “aryl” used alone or as part of a larger moiety as in “aralkyl,” “aralkoxy,” or “aryloxyalkyl,” refers to monocyclic or bicyclic ring systems having a total of five to fourteen ring members, wherein at least one ring in the system is aromatic and wherein each ring in the system contains 3 to 7 ring members. The term “aryl” may be used interchangeably with the term “aryl ring.” In certain embodiments of the present invention, “aryl” refers to an aromatic ring system which includes, but not limited to, phenyl, biphenyl, naphthyl, anthracyl and the like, which may bear one or more substituents. Also included within the scope of the term “aryl,” as it is used herein, is a group in which an aromatic ring is fused to one or more non-aromatic rings, such as indanyl, phthalimidyl, naphthimidyl, phenanthridinyl, or tetrahydronaphthyl, and the like.

The terms “heteroaryl” and “heteroar-,” used alone or as part of a larger moiety, e.g., “heteroaralkyl,” or “heteroaralkoxy,” refer to groups having 5 to 10 ring atoms, preferably 5, 6, or 9 ring atoms; having 6, 10, or 14 pi electrons shared in a cyclic array; and having, in addition to carbon atoms, from one to five heteroatoms. The term “heteroatom” refers to nitrogen, oxygen, or sulfur, and includes any oxidized form of nitrogen or sulfur, and any quaternized form of a basic nitrogen. Heteroaryl groups include, without limitation, thienyl, furanyl, pyrrolyl, imidazolyl, pyrazolyl, triazolyl, tetrazolyl, oxazolyl, isoxazolyl, oxadiazolyl, thiazolyl, isothiazolyl, thiadiazolyl, pyridyl, pyridazinyl, pyrimidinyl, pyrazinyl, indolizinyl, purinyl, naphthyridinyl, and pteridinyl. The terms “heteroaryl” and “heteroar-”, as used herein, also include groups in which a heteroaromatic ring is fused to one or more aryl, cycloaliphatic, or heterocyclyl rings, where the radical or point of attachment is on the heteroaromatic ring. Nonlimiting examples include indolyl, isoindolyl, benzothienyl, benzofuranyl, dibenzofuranyl, indazolyl, benzimidazolyl, benzthiazolyl, quinolyl, isoquinolyl, cinnolinyl, phthalazinyl, quinazolinyl, quinoxalinyl, 4H-quinolizinyl, carbazolyl, acridinyl, phenazinyl, phenothiazinyl, phenoxazinyl, tetrahydroquinolinyl, tetrahydroisoquinolinyl, and pyrido[2,3-b]-1,4-oxazin-3 (4H)-one. A heteroaryl group may be mono- or bicyclic. The term “heteroaryl” may be used interchangeably with the terms “heteroaryl ring,” “heteroaryl group,” or “heteroaromatic,” any of which terms include rings that are optionally substituted. The term “heteroaralkyl” refers to an alkyl group substituted by a heteroaryl, wherein the alkyl and heteroaryl portions independently are optionally substituted.

As used herein, the terms “heterocycle,” “heterocyclyl,” “heterocyclic radical,” and “heterocyclic ring” are used interchangeably and refer to a stable 5- to 7-membered monocyclic or 7-10-membered bicyclic heterocyclic moiety that is either saturated or partially unsaturated, and having, in addition to carbon atoms, one or more, preferably one to four, heteroatoms, as defined above. When used in reference to a ring atom of a heterocycle, the term “nitrogen” includes a substituted nitrogen. As an example, in a saturated or partially unsaturated ring having 0-3 heteroatoms selected from oxygen, sulfur or nitrogen, the nitrogen may be N (as in 3,4-dihydro-2H-pyrrolyl), NH (as in pyrrolidinyl), or ⁺NR (as in N-substituted pyrrolidinyl).

A heterocyclic ring can be attached to its pendant group at any heteroatom or carbon atom that results in a stable structure and any of the ring atoms can be optionally substituted.

Examples of such saturated or partially unsaturated heterocyclic radicals include, without limitation, tetrahydrofuranyl, tetrahydrothiophenyl, pyrrolidinyl, piperidinyl, pyrrolinyl, tetrahydroquinolinyl, tetrahydroisoquinolinyl, decahydroquinolinyl, oxazolidinyl, piperazinyl, dioxanyl, dioxolanyl, diazepinyl, oxazepinyl, thiazepinyl, morpholinyl, and quinuclidinyl. The terms “heterocycle,” “heterocyclyl,” “heterocyclyl ring,” “heterocyclic group,” “heterocyclic moiety,” and “heterocyclic radical,” are used interchangeably herein, and also include groups in which a heterocyclyl ring is fused to one or more aryl, heteroaryl, or cycloaliphatic rings, such as indolinyl, 3H-indolyl, chromanyl, phenanthridinyl, or tetrahydroquinolinyl. A heterocyclyl group may be mono- or bicyclic. The term “heterocyclylalkyl” refers to an alkyl group substituted by a heterocyclyl, wherein the alkyl and heterocyclyl portions independently are optionally substituted.

As used herein, the term “partially unsaturated” refers to a ring moiety that includes at least one double or triple bond. The term “partially unsaturated” is intended to encompass rings having multiple sites of unsaturation, but is not intended to include aryl or heteroaryl moieties, as herein defined.

As described herein, compounds of the invention may contain “optionally substituted” moieties. In general, the term “substituted,” whether preceded by the term “optionally” or not, means that one or more hydrogens of the designated moiety are replaced with a suitable substituent. Unless otherwise indicated, an “optionally substituted” group may have a suitable substituent at each substitutable position of the group, and when more than one position in any given structure may be substituted with more than one substituent selected from a specified group, the substituent may be either the same or different at every position. Combinations of substituents envisioned by this invention are preferably those that result in the formation of stable or chemically feasible compounds. The term “stable,” as used herein, refers to compounds that are not substantially altered when subjected to conditions to allow for their production, detection, and, in certain embodiments, their recovery, purification, and use for one or more of the purposes disclosed herein.

Each optional substituent on a substitutable carbon is a monovalent substituent independently selected from halogen; —(CH₂)₀₋₄R^{o}; —(CH₂)₀₋₄OR^{o}; —O(CH₂)₀₋₄R^{o}, —O—(CH₂)₀₋₄C(O)OR^{o}; —(CH₂)₀₋₄CH(OR^{o})₂; (CH₂)₀₋₄SR^{o}; —(CH₂)₀₋₄Ph, which may be substituted with R^{o}; (CH₂)₀₋₄₀ (CH₂)₀₋₁Ph which may be substituted with R^{o}; —CH═CHPh, which may be substituted with R^{o}; —(CH₂)₀₋₄₀ (CH₂)₀₋₁-pyridyl which may be substituted with R^{o}; —NO₂; —CN; —N₃; —(CH₂)₀₋₄N(R^{o})₂; —(CH₂)₀₋₄N(R^{o}C(O)R^{o}); —N(R^{o}C(S) R^{o}; —(CH₂)₀₋₄N(R^{o})C(O)NR^{o})₂; —N(R^{o}C)(S)NR^{o})₂; (CH₂)₀₋₄N(R^{o}C. (O) OR^{o}; —N(R^{o}N(R^{o}C(O)R^{o}); —N(R^{o}N(R^{o}C(O)NR^{o})₂; —N(R^{o}N(R^{o}C(O)OR^{o}; —(CH₂)₀₋₄C(O)R^{o}; —C(S)R^{o}; —(CH₂)₀₋₄C(O)OR^{o}; —(CH₂)₀₋₄C(O)SR^{o}; —(CH₂)₀₋₄C(O)OSiR^{o}₃; —(CH₂)₀₋₄OC(O)R^{o}; —OC(O)(CH₂)₀₋₄SR—, SC(S) SR^{o}; —(CH₂)₀₋₄SC(O)R^{o}; —(CH₂)₀₋₄C(O)NR^{o}₂; —C(S)NR^{o}₂; —C(S)SR^{o}; —SC(S)SR^{o}, —(CH₂)₀₋₄₀C(O)NR^{o}₂; —C(O)N(OR^{o})R^{o}; —C(O) C(O)R^{o}; —C(O)CH₂C(O)R^{o}; —C(NOR^{o})R^{o}; —(CH₂)₀₋₄SSR^{o}; —(CH₂)₀₋₄S(O)₂R^{o}; —(CH₂)₀₋₄S(O)₂OR^{o}; —(CH₂)₀₋₄OS(O)₂R^{o}; —S(O)₂NR^{o}₂; —S(O)(NR^{o})R^{o}; —S(O)₂N═C(NR^{o}₂)₂; —(CH₂)₀₋₄S(O)R^{o}; —N(R^{o}S(O)₂NR^{o})₂; —N(R^{o}S(O)₂R^{o}; —N(OR^{o})R^{o}; —C(NH)NR^{o}₂; —P(O)₂R^{o}; —P(O)R^{o}₂; —OP(O)R^{o}₂; —OP(O)(OR^{o})₂; SiR^{o}₃; —(C₁₋₄ straight or branched)alkylene)O—N(R^{o})₂; or —(C₁₋₄ straight or branched)alkylene) C(O)O—N(R^{o})₂.

Each R^{o} is independently hydrogen, C₁₋₆ aliphatic, —CH₂Ph, —O(CH₂)₀₋₁Ph, —CH₂-(5-6 membered heteroaryl ring), or a 5-6-membered saturated, partially unsaturated, or aryl ring having 0-4 heteroatoms independently selected from nitrogen, oxygen, or sulfur, or, notwithstanding the definition above, two independent occurrences of R^{o}, taken together with their intervening atom(s), form a 3-12-membered saturated, partially unsaturated, or aryl mono- or bicyclic ring having 0-4 heteroatoms independently selected from nitrogen, oxygen, or sulfur, which may be substituted by a divalent substituent on a saturated carbon atom of R^{o} selected from ═O and ═S; or each R^{o} is optionally substituted with a monovalent substituent independently selected from halogen, —(CH₂)₀₋₂R^{•}, -(haloR^{•}), —(CH₂)₀₋₂OH, —(CH₂)₀₋₂OR^{•}, —(CH₂)₀₋₂CH(OR^{•})₂; —O(haloR^{•}), —CN, —N₃, (CH₂)₀₋₂C(O)R^{•}, —(CH₂)₀₋₂C(O)OH, —(CH₂)₀₋₂C(O)OR^{•}, —(CH₂)₀₋₂SR^{•}, —(CH₂)₀₋₂SH, —(CH₂)₀₋₂NH₂, —(CH₂)₀₋₂NHR^{•}, —(CH₂)₀₋₂NR^{•}₂, —NO₂, —SiR^{•}₃, —OSiR^{•}₃, —C(O)SR^{•}, —(C₁₋₄ straight or branched alkylene)C(O)OR^{•}, or —SSR^{•}.

Each R^{•} is independently selected from C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur, and wherein each R^{•} is unsubstituted or where preceded by halo is substituted only with one or more halogens; or wherein an optional substituent on a saturated carbon is a divalent substituent independently selected from ═O, ═S, ═NNR*2, ═NNHC(O)R*, ═NNHC(O)OR*, ═NNHS(O)₂R*, ═NR*, ═NOR*, —O(C(R*₂))₂₋₃O—, or —S(C(R*₂))₂₋₃S—, or a divalent substituent bound to vicinal substitutable carbons of an “optionally substituted” group is —O(CR*2)₂₋₃O—, wherein each independent occurrence of R* is selected from hydrogen, C₁₋₆ aliphatic or an unsubstituted 5-6-membered saturated, partially unsaturated, or aryl ring having 0-4 heteroatoms independently selected from nitrogen, oxygen, or sulfur.

When R* is C₁₋₆ aliphatic, R* is optionally substituted with halogen, —R^{•}, -(haloR^{•}), —OH, —OR^{•}, —O(haloR^{•}), —CN, —C(O) OH, —C(O) OR*, —NH₂, —NHR^{•}, —NR^{•}₂, or —NO₂, wherein each R^{•} is independently selected from C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur, and wherein each R^{•} is unsubstituted or where preceded by halo is substituted only with one or more halogens.

An optional substituent on a substitutable nitrogen is independently —R^{†}, —NR^{†}₂, —C(O)R^{†}, —C(O)OR^{†}, —C(O)C(O)R^{†}, —C(O)CH₂C(O)R^{†}, —S(O)₂R^{†}, —S(O)₂NR^{†}₂, —C(S)NR^{†}₂, —C(NH)NR^{†}₂, or —N(R^{†})S(O)₂R^{†}; wherein each Rt is independently hydrogen, C₁₋₆ aliphatic, unsubstituted —OPh, or an unsubstituted 5-6-membered saturated, partially unsaturated, or aryl ring having 0-4 heteroatoms independently selected from nitrogen, oxygen, or sulfur, or, two independent occurrences of Rt, taken together with their intervening atom(s) form an unsubstituted 3-12-membered saturated, partially unsaturated, or aryl mono- or bicyclic ring having 0-4 heteroatoms independently selected from nitrogen, oxygen, or sulfur; wherein when Rt is C₁₋₆ aliphatic, R^{†} is optionally substituted with halogen, —R^{•}, -(haloR^{•}), —OH, —OR′, —O(haloR^{•}), —CN, —C(O)OH, —C(O)OR^{•}, —NH₂, —NHR^{•}, —NR^{•}₂, or —NO₂, wherein each R^{•} is independently selected from C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur, and wherein each R^{•} is unsubstituted or where preceded by halo is substituted only with one or more halogens.

As used herein, the term “pharmaceutically acceptable salt” refers to those salts which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts are well known in the art. For example, S. M. Berge et al., describe pharmaceutically acceptable salts in detail in J. Pharmaceutical Sciences, 1977, 66, 1-19, incorporated herein by reference. Pharmaceutically acceptable salts of the compounds of this invention include those derived from suitable inorganic and organic acids and bases. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other methods used in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, pivalate, propionate, stearate, succinate, sulfate, tartrate, thiocyanate, p-toluenesulfonate, undecanoate, valerate salts, and the like.

Salts derived from appropriate bases include alkali metal, alkaline earth metal, ammonium and N⁺(C₁₋₄alkyl)₄ salts. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, loweralkyl sulfonate and aryl sulfonate.

Unless otherwise stated, structures depicted herein are also meant to include all isomeric (e.g., enantiomeric, diastereomeric, and geometric (or conformational)) forms of the structure; for example, the R and S configurations for each asymmetric center, Z and E double bond isomers, and Z and E conformational isomers. Therefore, single stereochemical isomers as well as enantiomeric, diastereomeric, and geometric (or conformational) mixtures of the present compounds are within the scope of the invention. Unless otherwise stated, all tautomeric forms of the compounds of the invention are within the scope of the invention. Additionally, unless otherwise stated, structures depicted herein are also meant to include compounds that differ only in the presence of one or more isotopically enriched atoms. For example, compounds having the present structures including the replacement of hydrogen by deuterium or tritium, or the replacement of a carbon by a ¹³C- or ¹⁴C-enriched carbon are within the scope of this invention. Such compounds are useful, for example, as analytical tools, as probes in biological assays, or as therapeutic agents in accordance with the present invention. In certain embodiments, a warhead moiety, R¹, of a provided compound comprises one or more deuterium atoms.

As used herein, the term “inhibitor” is defined as a compound that binds to and/or inhibits BTK and/or CXCR4 with measurable affinity. In certain embodiments, an inhibitor has an IC₅₀ and/or binding constant of less than about 100 μM, less than about 50 μM, less than about 1 μM, less than about 500 nM, less than about 100 nM, less than about 10 nM, or less than about 1 nM.

The terms “measurable affinity” and “measurably inhibit,” as used herein, means a measurable change in BTK and/or CXCR4 activity between a sample comprising a compound of the present invention, or composition thereof, and BTK and/or CXCR4, and an equivalent sample comprising BTK and/or CXCR4, in the absence of said compound, or composition thereof.

The terms “administration,” “administering,” “treating,” and “treatment” herein, when applied to an animal, human, experimental subject, cell, tissue, organ, or biological fluid, mean contact of an exogenous pharmaceutical, therapeutic, diagnostic agent, or composition to the animal, human, subject, cell, tissue, organ, or biological fluid. Treatment of a cell encompasses contact of a reagent to the cell, as well as contact of a reagent to a fluid, where the fluid is in contact with the cell. The term “administration” and “treatment” also means in vitro and ex vivo treatments, e.g., of a cell, by a reagent, diagnostic agent, binding compound, or by another cell. The term “subject” herein includes any organism, preferably an animal, more preferably a mammal (e.g., rat, mouse, dog, cat, and rabbit) and most preferably a human.

The term “effective amount” or “therapeutically effective amount” refers to an amount of the active ingredient, such as compound that, when administered to a subject for treating a disease, or at least one of the clinical symptoms of a disease or disorder, is sufficient to affect such treatment for the disease, disorder, or symptom. The “therapeutically effective amount” can vary with the compound, the disease, disorder, and/or symptoms of the disease or disorder, severity of the disease, disorder, and/or symptoms of the disease or disorder, the age of the subject to be treated, and/or the weight of the subject to be treated. An appropriate amount in any given instance can be apparent to those skilled in the art or can be determined by routine experiments. In some embodiments, “therapeutically effective amount” is an amount of at least one compound and/or at least one stereoisomer thereof, and/or at least one pharmaceutically acceptable salt thereof disclosed herein effective to “treat” as defined herein, a disease or disorder in a subject. In the case of combination therapy, the “therapeutically effective amount” refers to the total amount of the combination objects for the effective treatment of a disease, a disorder or a condition.

As used herein, “about” means that the stated value or range may vary by up to 10% from the stated value or range. For example, “about” 5.0 means 5.0±0.5, and “about 5.0-10.0” means 4.5-10.5.

### 3. Description of Exemplary Embodiments

In one aspect, the present invention provides a compound of formula I:

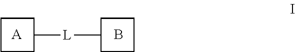

or a pharmaceutically acceptable salt thereof, wherein:
[symbol] is a small molecule BTK inhibitor;
[symbol] is a small molecule or peptide CXCR4 inhibitor, or a CXCR4 antibody; and
-L- is a covalent bond or a bivalent linker;
wherein -L- is attached via a covalent bond to each of [symbol] and [symbol] at a carbon, nitrogen, or oxygen atom, and each of the covalent bonds independently replaces a hydrogen on the carbon, nitrogen, or oxygen atom.

In some embodiments, [symbol] is a compound or moiety that exhibits activity in a BTK screening assay, such as those described herein (i.e., a BTK-inhibiting compound or BTK inhibitor). In some embodiments, [symbol] is a compound or moiety that exhibits activity in a CXCR4 screening assay, such as those described herein (i.e., a CXCR4-inhibiting compound or CXCR4 inhibitor).

In some embodiments, the BTK inhibitor is LOXO-305 (pirtobrutinib), tirabrutinib (also known as (R)-6-amino-9-(1-(but-2-ynoyl) pyrrolidin-3-yl)-7-(4-phenoxyphenyl)-7,9-dihydro-8H-purin-8-one), zanubrutinib, ibrutinib, acalabrutinib, evobrutinib, fenebrutinib, poseltinib, vecabrutinib, tirabrutinib, or spebrutinib.

In some embodiments, the BTK inhibitor is ibrutinib:

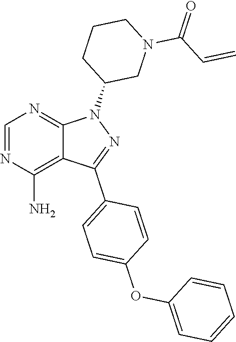 [SMILES: C=CC(=O)N1CCC[C@@H](n2nc(-c3ccc(Oc4ccccc4)cc3)c3c(N)ncnc32)C1]

or an analog thereof or a pharmaceutically acceptable salt thereof.

Ibrutinib has been approved as a single agent to treat WM in both US and European Union (EU) (ibrutinib (IMBRUVICA®)). In the US, ibrutinib can be used in any line of treatment while in the EU, ibrutinib is approved for patients who have received at least one prior therapy, or in first-line treatment for patients unsuitable for chemo-immunotherapy. The ibrutinib monotherapy and rituximab combination pivotal trials have identified genetic mutation patients who have not benefited to the same extent of those patients without genetic mutations. One specific population identified with a remaining unmet medical need is the double mutation, MYD88^{L265P} CXCR4^{WHIM} population, estimated at approximately 27% of the WM population (Treon 2015 [1], Treon 2018 [1]; Hunter 2014). Patients with the double mutations had a significantly reduced VGPR (9.5%) as compared with patients with the MYD88^{L265P} CXCR4^{WT} mutations (44.4%) (Treon 2015 [2]; Treon 2018 [2]).

Ibrutinib and its analogs are described in U.S. Pat. No. 7,514,444, the disclosure of which is incorporated herein by reference in its entirety. Additional representative examples of BTK inhibitors are set forth below.

In some embodiments, the BTK inhibitor is BIIB-068, which has the following structure:

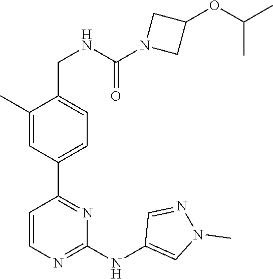 [SMILES: Cc1cc(-c2ccnc(Nc3cnn(C)c3)n2)ccc1CNC(=O)N1CC(OC(C)C)C1]

In some embodiments, the BTK inhibitor is zanubrutinib, or an analog thereof. Zanubrutinib is also known as (7S)-2-(4-phenoxyphenyl)-7-(1-prop-2-enoylpiperidin-4-yl)-4,5,6,7-tetrahydropyrazolo[1,5-a]pyrimidine-3-carboxamide. This drug and its analogs are described in U.S. Pat. No. 9,447,106, the disclosure of which is incorporated herein by reference in its entirety.

In some embodiments, the BTK inhibitor is LOXO-305 (pirtobrutinib or LY3527727). This compound is described in Mato et al. (2021) The Lancet 397:892-901.

In some embodiments, the BTK inhibitor is acalabrutinib, or an analog thereof. Acalabrutinib is also known as 4-[8-amino-3-[(2S)-1-but-2-ynoylpyrrolidin-2-yl]imidazo[1,5-a]pyrazin-1-yl]-N-pyridin-2-ylbenzamide. This drug and its analogs are described in U.S. Pat. No. 9,290,504, the disclosure of which is incorporated herein by reference in its entirety.

In some embodiments, the BTK inhibitor is LCB 03-0110 dihydrochloride, or an analog thereof or a salt thereof such as a dihydrochloride salt. LCB 03-0110 is also known as 3-[[2-[3-(4-morpholinylmethyl)phenyl]thieno[3,2-b]pyridin-7-yl]amino]phenol dihydrochloride. This compound is described in Sun et al., J. Pharmacol. Exp. Ther. 340(3):510-519 (2012), the disclosure of which is incorporated herein by reference in its entirety.

In some embodiments, the BTK inhibitor is LFM-A13, or an analog thereof. LFM-A13 is also known as 2-cyano-N-(2,5-dibromophenyl)-3-hydroxy-2-butenamide. This compound is described in Vassilev et al., J. Biol. Chem. 274(3):1646-1656 (1999), the disclosure of which is incorporated herein by reference in its entirety.

In some embodiments, the BTK inhibitor is PCI 29732, or an analog thereof. PCI 29732 is also known as 1-cyclopentyl-3-(4-phenoxyphenyl)-1H-pyrazolo[3,4-d]pyrimidin-4-amine. This compound is described in Honigberg et al., Proc. Natl. Acad. Sci. USA 107(29):13075-13080 (2010), the disclosure of which is incorporated herein by reference in its entirety.

In some embodiments, the BTK inhibitor is PF 06465469, or an analog thereof. PF 06465469 is also known as (R)-3-(1-(1-acryloylpiperidin-3-yl)-4-amino-1H-pyrazolo[3,4-d]pyrimidin-3-yl)-N-(3-methyl-4-(1-methylethyl)) benzamide. This compound is described in Zapf et al., J. Med. Chem. 55(22):10047-10063 (2012), the disclosure of which is incorporated herein by reference in its entirety.

In some embodiments, the BTK inhibitor is (−)-Terreic acid, or an analog thereof. (−)-Terreic acid is also known as (1R,6S)-3-hydroxy-4-methyl-7-oxabicyclo[4.1.0]hept-3-ene-2,5-dione, is as follows. This compound is described in Kawakami et al., Proc. Natl. Acad. Sci. USA 96(5):2227-2232 (1999), the disclosure of which is incorporated herein by reference in its entirety.

In some embodiments, the BTK inhibitor BMX-IN-1, or an analog thereof. BMX-IN-1 is known as N-[2-Methyl-5-[9-[4-[(methylsulfonyl)amino]phenyl]-2-oxobenzo[h]-1,6-naphthyridin-1 (2H)-yl]phenyl]-2-propenamide. This compound is described in Li et al., Oncotarget. 8(30):49238-49252 (2017), the disclosure of which is incorporated herein by reference in its entirety.

In some embodiments, the BTK inhibitor is ARQ-531, or an analog thereof. ARQ-531 is also known as (2-chloro-4-phenoxyphenyl) (4-(((3R,6S)-6-(hydroxymethyl)tetrahydro-2H-pyran-3-yl)amino)-7H-pyrrolo[2,3-d]pyrimidin-5-yl) methanone. This compound is described in Elgamal et al., J. Hematol. Oncol. 13:8 (2020), the disclosure of which is incorporated herein by reference in its entirety.

In some embodiments, the BTK inhibitor is BI-BTK-1, or an analog thereof. BI-BTK-1 is also known as 5-Amino-3-(4-phenoxyphenyl)-1-((2r,4s)-6-((E)-4-(piperidin-1-yl) but-2-enoyl)-6-azaspiro[3.4]octan-2-yl)-1H-pyrazole-4-carboxamide. This compound is described in Chalmers et al., Arthritis Res. Ther. 20(1):10 (2018), the disclosure of which is incorporated herein by reference in its entirety.

In some embodiments, the BTK inhibitor is BMS-986142, or an analog thereof. BMS-986142 is also known as 1H-Carbazole-8-carboxamide, (2S)-6-fluoro-5-[3-(8-fluoro-1-methyl-2,4-dioxo-1,2,3,4-tetrahydroquinazolin-3-yl)-2-methylphenyl]-2-(2-hydroxypropan-2-yl)-2,3,4,9-tetrahydro-1H-carbazole-8-carboxamide. This compound is described in Watterson et al., J. Med. Chem. 59(19):9173-9200, the disclosure of which is incorporated herein by reference in its entirety.

In some embodiments, the BTK inhibitor is CGI-1746, or an analog thereof. CGI-1746 is also known as 4-tert-butyl-N-[2-methyl-3-[4-methyl-6-[4-(morpholine-4-carbonyl)anilino]-5-oxopyrazin-2-yl]phenyl]benzamide. This compound is described in Di Paolo et al., Nat. Chem. Biol.7(1):51-50 the disclosure of which is incorporated herein by reference in its entirety.

In some embodiments, the BTK inhibitor is evobrutinib, or an analog thereof. Evobrutinib is also known as 1-[4-[[[6-amino-5-(4-phenoxyphenyl)pyrimidin-4-yl]amino]methyl]piperidin-1-yl]prop-2-en-1-one. This compound is described in Crawford et al., J. Med. Chem. 61(6):2227-2245, the disclosure of which is incorporated herein by reference in its entirety.

In some embodiments, the BTK inhibitor is fenebrutinib, or an analog thereof. Fenebrutinib is also known as 10-[3-(hydroxymethyl)-4-[1-methyl-5-[[5-[(2S)-2-methyl-4-(oxetan-3-yl)piperazin-1-yl]pyridin-2-yl]amino]-6-oxopyridin-3-yl]pyridin-2-yl]-4,4-dimethyl-1,10-diazatricyclo[6.4.0.02,6]dodeca-2 (6),7-dien-9-one. This compound is described in Crawford et al., WO 2013/067274, the disclosure of which is incorporated herein by reference in its entirety.

In some embodiments, the BTK inhibitor is GDC-0834, or an analog thereof. GDC-0834 is also known as (R)—N-(3-(6-((4-(1,4-dimethyl-3-oxopiperazin-2-yl)phenyl)amino)-4-methyl-5-oxo-4,5-dihydropyrazin-2-yl)-2-methylphenyl)-4,5,6,7-tetrahydrobenzo[b]thiophene-2-carboxamide. This compound is described in Liu et al., J. Pharmacol. Exp. Ther. 338 (1): 154-163, the disclosure of which is incorporated herein by reference in its entirety.

In some embodiments, the BTK inhibitor is olmutinib, or an analog thereof. Olmutinib is also known as N-[3-[2-[4-(4-methylpiperazin-1-yl) anilino]thieno[3,2-d]pyrimidin-4-yl]oxyphenyl]prop-2-enamide. This compound is described in Cha et al., U.S. Pat. No. 9,345,719, the disclosure of which is incorporated herein by reference in its entirety.

In some embodiments, the BTK inhibitor is PLS-123, or an analog thereof. PLS-123 is also known as N-(2-((3-(2-acrylamidoacetamido)phenyl)amino) pyrimidin-5-yl)-2-methyl-5-(3-(trifluoromethyl)benzamido)benzamide. This compound is described in Ding et al., Oncotarget. 6(17):15122-15136 (2015), the disclosure of which is incorporated herein by reference in its entirety.

In some embodiments, the BTK inhibitor is PRN1008, or an analog thereof. PRN1008 is also known as (S,E)-2-(3-(4-amino-3-(2-fluoro-4-phenoxyphenyl)-1H-pyrazolo[3,4-d]pyrimidin-1-yl)piperidine-1-carbonyl)-4-methyl-4-(4-(oxetan-3-yl)piperazin-1-yl) pent-2-enenitrile. This compound is described in Smith et al., Br. J. Clin. Pharmacol. 83 (11): 2367-2376 (2017), the disclosure of which is incorporated herein by reference in its entirety.

In some embodiments, the BTK inhibitor is RN-486, or an analog thereof. RN-486 is also known as 6-cyclopropyl-8-fluoro-2-(2-hydroxymethyl-3-{1-methyl-5-[5-(4-methyl-piperazin-1-yl)-pyridin-2-ylamino]-6-oxo-1,6-dihydro-pyridin-3-yl}-phenyl)-2H-isoquinolin-1-one. This compound is described in Zhao et al., Bioorg. Med. Chem. 23(15):4344-4353 (2015), the disclosure of which is incorporated herein by reference in its entirety.

In some embodiments, the BTK inhibitor is spebrutinib, or an analog thereof.

Spebrutinib is also known as N-[3-[[5-fluoro-2-[4-(2-methoxyethoxy) anilino]pyrimidin-4-yl]amino]phenyl]prop-2-enamide. This compound is described in Chen et al., U.S. 2019/144451, the disclosure of which is incorporated herein by reference in its entirety.

In some embodiments, the BTK inhibitor is tirabrutinib, or an analog thereof. Tirabrutinib is also known as (R)-6-amino-9-(1-(but-2-ynoyl) pyrrolidin-3-yl)-7-(4-phenoxyphenyl)-7,9-dihydro-8H-purin-8-one. This compound is described in Izumi et al., U.S. 2018/193337, the disclosure of which is incorporated herein by reference in its entirety.

In some embodiments, the BTK inhibitor is vecabrutinib, or an analog thereof. Vecabrutinib is also known as (3R,4S)-1-(6-amino-5-fluoropyrimidin-4-yl)-3-[(3R)-3-[3-chloro-5-(trifluoromethyl) anilino]-2-oxopiperidin-1-yl]piperidine-4-carboxamide.

In one aspect, the present invention provides a compound of formula I:

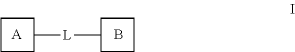

or a pharmaceutically acceptable salt thereof, wherein [symbol] is a BTK inhibitor selected from the following:

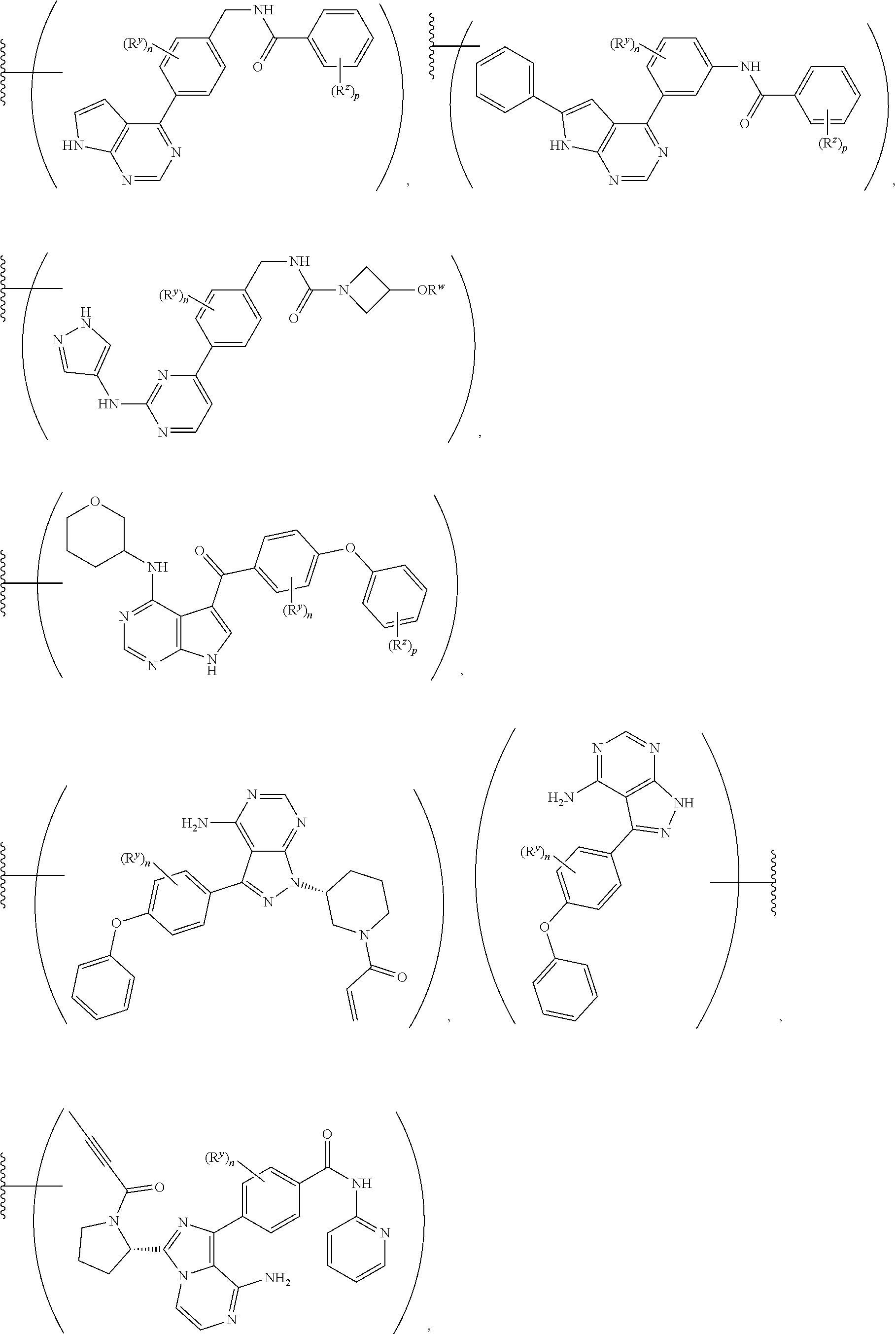

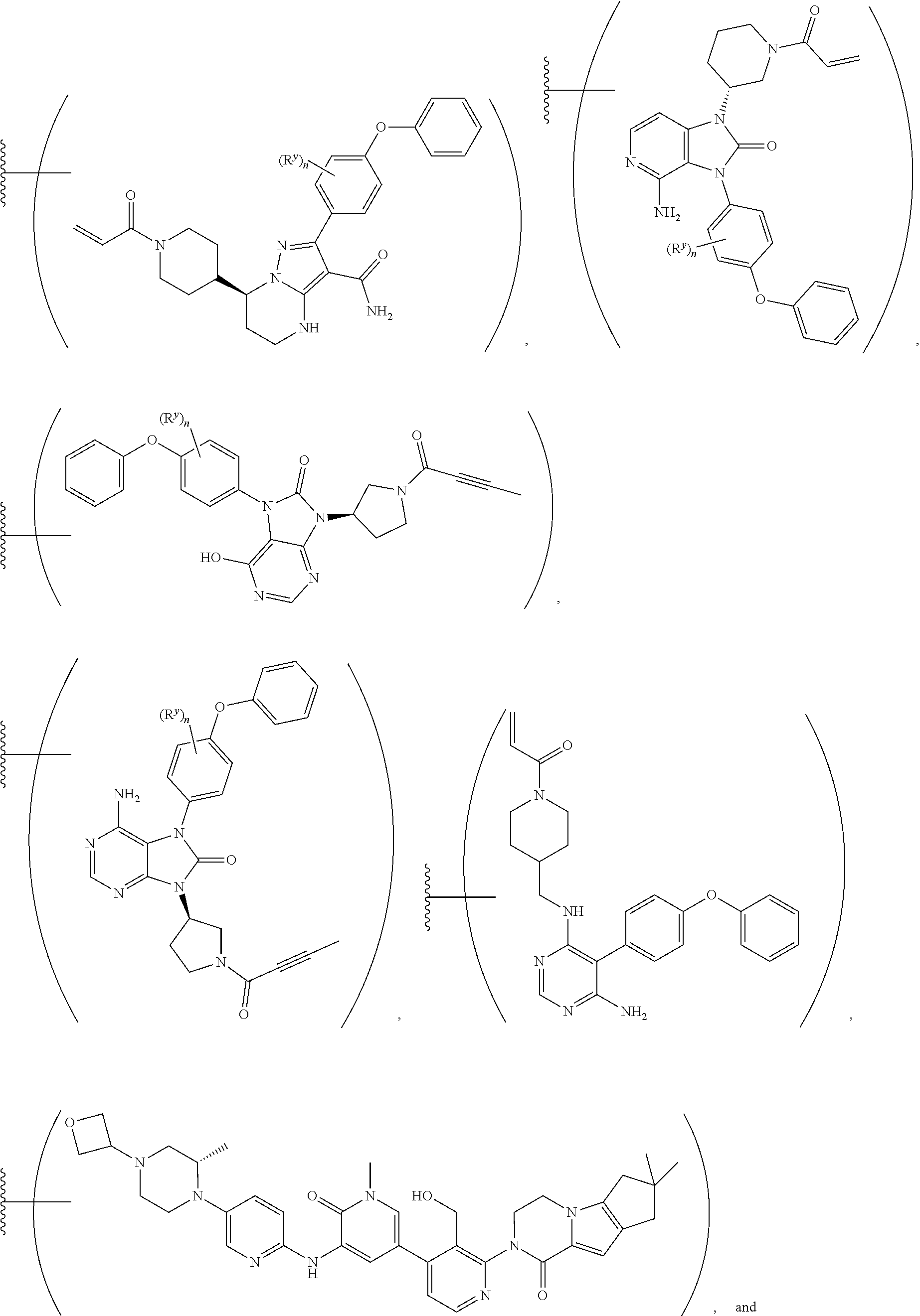

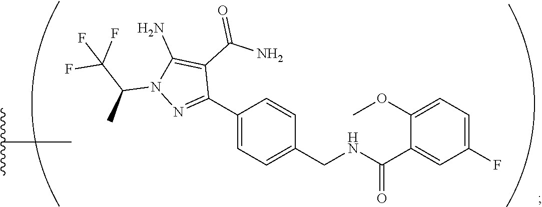

wherein,
   R^{w} is hydrogen, or C₁₋₇ aliphatic optionally substituted with 1-5 halogens;
   R^{y} is hydrogen, halogen, or C₁₋₇ aliphatic optionally substituted with 1-5 halogens;
   R^{z} is hydrogen, halogen, or C₁₋₇ aliphatic optionally substituted with 1-5 halogens;
-L- is one of the following:
   (a) a C₁₋₈ bivalent straight or branched, saturated or unsaturated hydrocarbon chain wherein 1-4 methylene units of the chain are independently and optionally replaced with —O—, —C(O)—, —C(S)—, -Cy-, —C(R^{A})₂—, —CH(R^{A})—, —CH(OR)—, N(R)—, —S—, —S(O)—, or —S(O)₂—; or
   (b) a covalent bond;
   each R^{A} is independently hydrogen, halogen, —CN, optionally substituted group selected from C₁₋₆ aliphatic; phenyl; an 8-10 membered bicyclic aryl ring, a 3-7 membered saturated or partially unsaturated monocyclic carbocyclic ring; a 4-8 membered saturated or partially unsaturated monocyclic heterocyclic ring having 1-2 heteroatoms independently selected from nitrogen, oxygen, and sulfur; a 5-6 membered monocyclic heteroaryl ring having 1-4 heteroatoms independently selected from nitrogen, oxygen, and sulfur; and an 8-10 membered bicyclic heteroaryl ring having 1-5 heteroatoms independently selected from nitrogen, oxygen, and sulfur; or
   two R^{A} groups on the same carbon are optionally taken together, with the carbon they are attached to, to form:
      an optionally substituted 3-10 membered monocyclic or bicyclic saturated, or partially unsaturated, carbocyclic ring; or
      an optionally substituted 3-10 membered monocyclic or bicyclic saturated, or partially unsaturated, heterocyclic ring, having 0-3 heteroatoms independently selected from nitrogen, oxygen, and sulfur;
   each R is independently hydrogen, or an optionally substituted group selected from C₁₋₆ aliphatic; phenyl; an 8-10 membered bicyclic aryl ring, a 3-7 membered saturated or partially unsaturated monocyclic carbocyclic ring; a 4-8 membered saturated or partially unsaturated monocyclic heterocyclic ring having 1-2 heteroatoms independently selected from nitrogen, oxygen, and sulfur; a 5-6 membered monocyclic heteroaryl ring having 1-4 heteroatoms independently selected from nitrogen, oxygen, and sulfur; and an 8-10 membered bicyclic heteroaryl ring having 1-5 heteroatoms independently selected from nitrogen, oxygen, and sulfur; or
      two R groups on the same nitrogen are optionally taken together with the nitrogen to form an optionally substituted 4-7 membered monocyclic saturated, partially unsaturated, or heteroaryl ring having, in addition to the nitrogen, 0-3 heteroatoms independently selected from nitrogen, oxygen, and sulfur;
   each -Cy- is independently an optionally substituted bivalent ring selected from a 4-7 membered saturated or partially unsaturated heterocyclylenyl having 1-2 heteroatoms independently selected from nitrogen, oxygen, and sulfur, phenylenyl, a 3-7 membered saturated or partially unsaturated carbocyclylenyl, or a 5-6 membered heteroarylenyl having 1-4 heteroatoms independently selected from nitrogen, oxygen, and sulfur;
[symbol] is a CXCR4 inhibitor selected from the following:

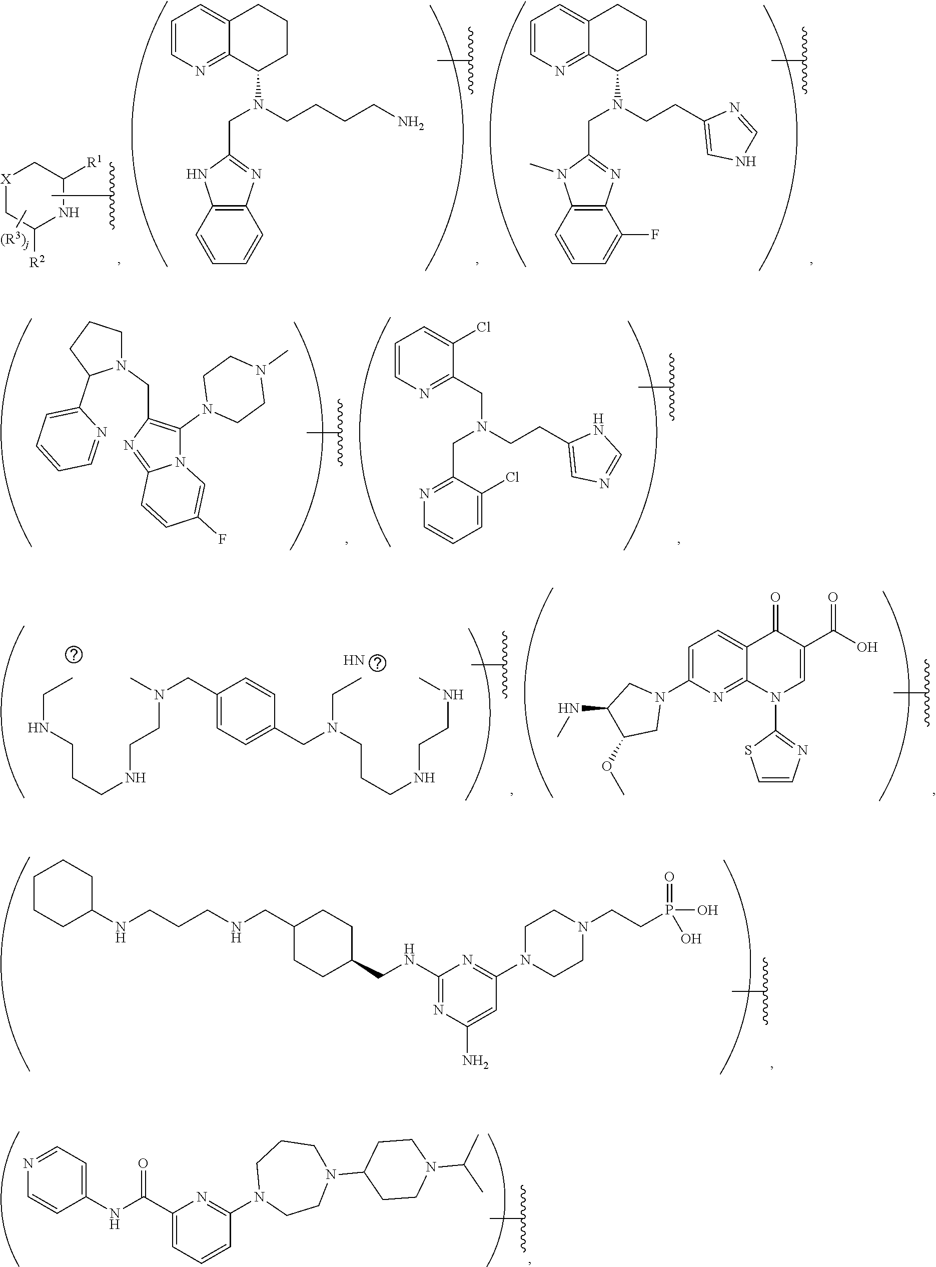

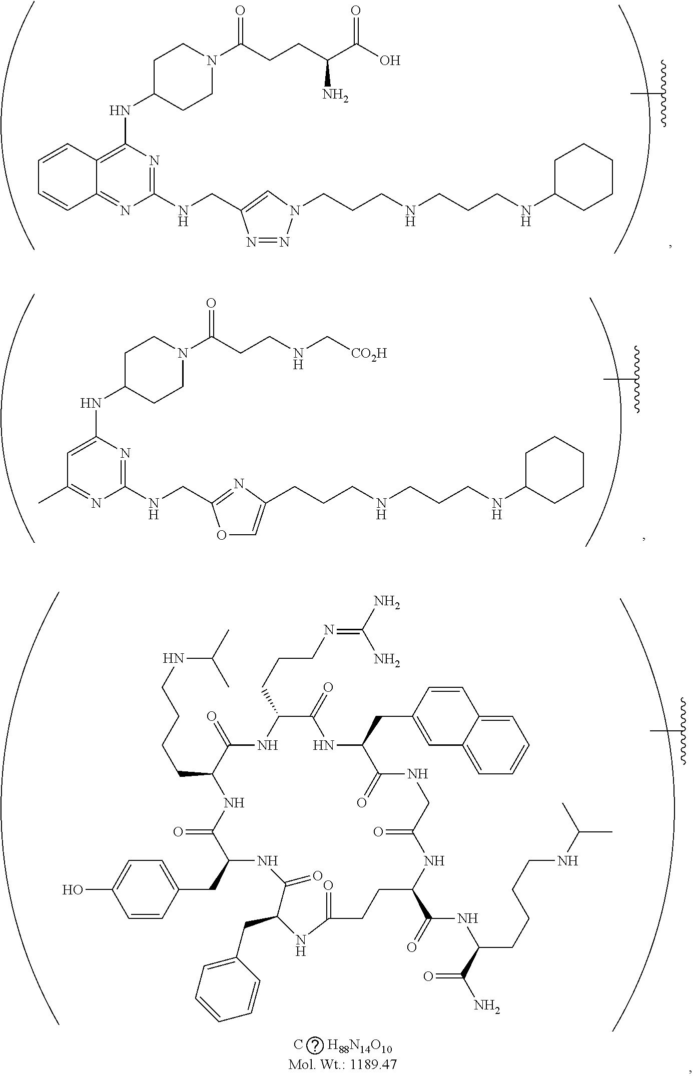

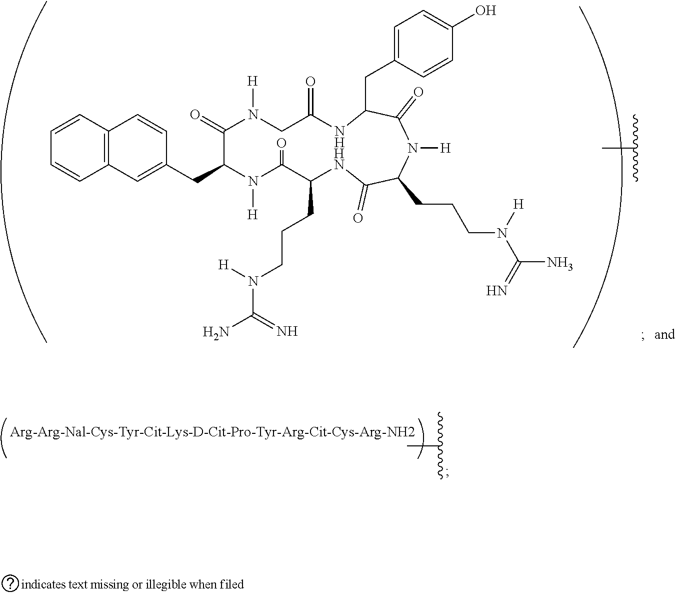

wherein,
—X— is carbon, nitrogen, oxygen, sulfur, SO, or SO₂;
each of R¹ and R² is independently one of the following:
   (a) C₁₋₇ aliphatic; phenyl; a 5-6 membered monocyclic heteroaryl ring having 1-4 heteroatoms independently selected from nitrogen, oxygen, and sulfur; a 3-7 membered saturated or partially unsaturated monocyclic carbocyclic ring; a 4-8 membered saturated or partially unsaturated monocyclic heterocyclic ring having 1-2 heteroatoms independently selected from nitrogen, oxygen, and sulfur; a 5-10 membered saturated or partially unsaturated bridged bicyclic ring having 0-3 heteroatoms independently selected from nitrogen, oxygen, and sulfur; a 5-10 membered saturated or partially unsaturated spirocyclic ring having 0-3 heteroatoms independently selected from nitrogen, oxygen, and sulfur; or an 8-10 membered partially aromatic or heteroaromatic bicyclic heterocyclic ring having 1-4 heteroatoms independently selected from nitrogen, oxygen, and sulfur; each of which is substituted with m instances of R′; or
   (b) —C(O)N(R″)₂, or —C(O) OR″;
      each occurrence of R′ is independently hydrogen, halogen, or a C₁₋₆ aliphatic group optionally substituted with 1 to 5 halogens;
      each occurrence of R″ is independently hydrogen, or a C₁₋₆ aliphatic group optionally substituted with 1 to 5 halogens;
each R³ is independently —N(R)₂, —OR, or C₁₋₇ aliphatic optionally substituted with 1-5 groups independently selected from halogen, —N(R)₂, or —OR, and wherein 1 or 2 methylene units of the aliphatic group are optionally replaced by —N(R)— or a methyl group is optionally replaced by —N(R)₂;
j is 0, 1, or 2;
m is 0, 1, 2, 3, or 4;
n is 0, 1, 2, 3, or 4;
p is 0, 1, 2, 3, 4, or 5; and
wherein -L- is attached via a covalent bond to each of [symbol] and [symbol] at a carbon, nitrogen, or oxygen atom, and each of the covalent bonds independently replaces a hydrogen on the carbon, nitrogen, or oxygen atom.

In some embodiments, the present invention provides a compound of formula I, or a pharmaceutically acceptable salt thereof, wherein [symbol] is a CXCR4 inhibitor selected from the following:

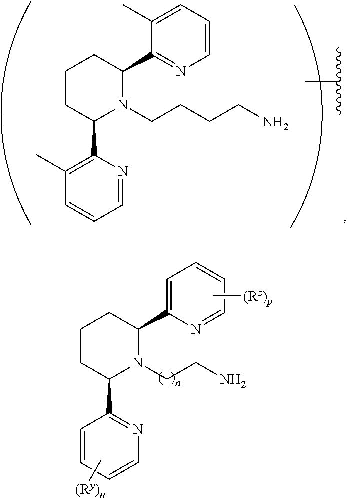

wherein n is 1-3,

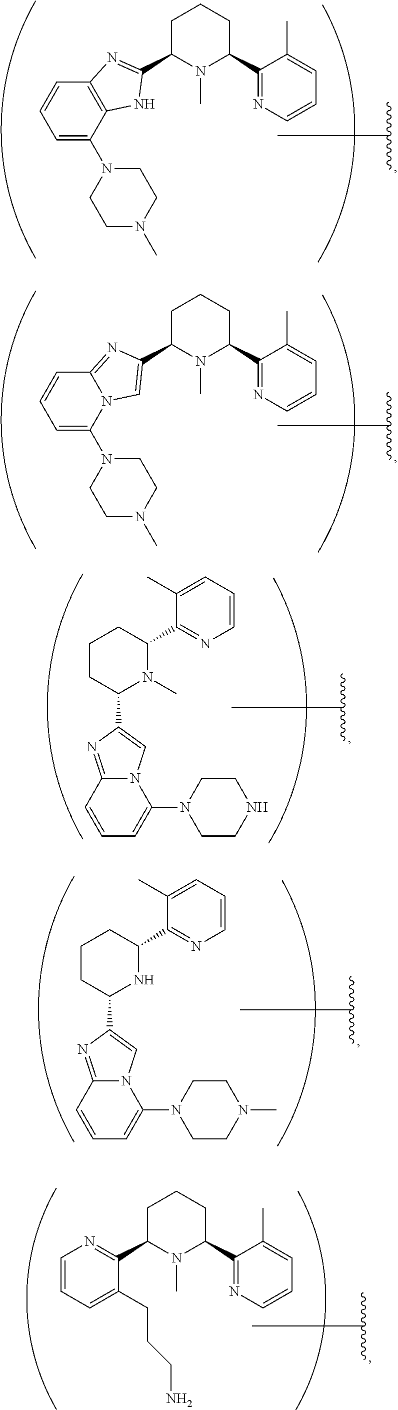

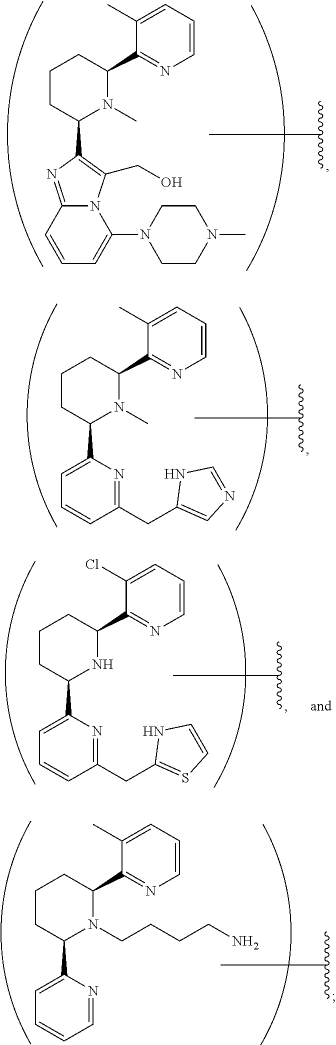

wherein -L- is attached via a covalent bond to each of [symbol] and [symbol] at a carbon, nitrogen, or oxygen atom, and each of the covalent bonds independently replaces a hydrogen on the carbon, nitrogen, or oxygen atom.

In some embodiments, the present invention provides a compound of formula I or a pharmaceutically acceptable salt thereof, wherein [symbol] is a CXCR4 inhibitor of Formula B-a:

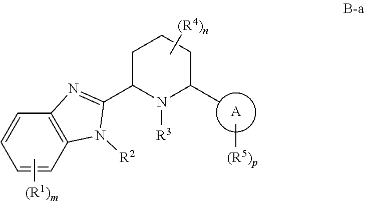

or a pharmaceutically acceptable salt thereof, wherein:
Ring A is a 3-8 membered saturated or partially unsaturated monocyclic carbocyclic ring, phenyl, an 8-10 membered bicyclic aromatic carbocyclic ring, a 4-8 membered saturated or partially unsaturated monocyclic heterocyclic ring having 1-2 heteroatoms independently selected from nitrogen, oxygen, or sulfur, a 5-6 membered monocyclic heteroaromatic ring having 1-4 heteroatoms independently selected from nitrogen, oxygen, or sulfur, or an 8-10 membered bicyclic heteroaromatic ring having 1-5 heteroatoms independently selected from nitrogen, oxygen, or sulfur;
each R¹ is independently R, halogen, —CN, —OR, —N(R)₂, —NO₂, —N₃, —SR, or -L¹-R⁶;
each R is independently hydrogen or an optionally substituted group selected from C₁₋₆ aliphatic, a 3-8 membered saturated or partially unsaturated monocyclic carbocyclic ring, phenyl, an 8-10 membered bicyclic aromatic carbocyclic ring, a 4-8 membered saturated or partially unsaturated monocyclic heterocyclic ring having 1-2 heteroatoms independently selected from nitrogen, oxygen, or sulfur, a 5-6 membered monocyclic heteroaromatic ring having 1-4 heteroatoms independently selected from nitrogen, oxygen, or sulfur, or an 8-10 membered bicyclic heteroaromatic ring having 1-5 heteroatoms independently selected from nitrogen, oxygen, or sulfur;
each L¹ and L² is independently a covalent bond or a C₁₋₈ bivalent straight or branched hydrocarbon chain wherein 1, 2, or 3 methylene units of the chain are independently and optionally replaced with —O—, —C(O)—, —C(O)O—, —OC(O)—, —N(R)—, —C(O)N(R)—, —(R)NC(O)—, —OC(O)N(R)—, —(R)NC(O)O—, —N(R)C(O)N(R)—, —S—, —SO—, —SO₂—, —SO₂N(R)—, —(R)NSO₂—, —C(S)—, —C(S)O—, —OC(S)—, —C(S) N(R)—, —(R)NC(S)—, —(R)NC(S) N(R)—, or -Cy-;
each -Cy- is independently a bivalent optionally substituted 3-8 membered saturated or partially unsaturated monocyclic carbocyclic ring, optionally substituted phenylene, an optionally substituted 4-8 membered saturated or partially unsaturated monocyclic heterocyclic ring having 1-3 heteroatoms independently selected from nitrogen, oxygen, or sulfur, an optionally substituted 5-6 membered monocyclic heteroaromatic ring having 1-4 heteroatoms independently selected from nitrogen, oxygen, or sulfur, an optionally substituted 8-10 membered bicyclic or bridged bicyclic saturated or partially unsaturated heterocyclic ring having 1-5 heteroatoms independently selected from nitrogen, oxygen, or sulfur, or an optionally substituted 8-10 membered bicyclic or bridged bicyclic heteroaromatic ring having 1-5 heteroatoms independently selected from nitrogen, oxygen, or sulfur;
R² is hydrogen, -L²-R⁶, or optionally substituted C₁₋₈ aliphatic;
R³ is hydrogen, optionally substituted C₁₋₆ aliphatic, or -L³-R⁶;
L³ is a C₁₋₆ bivalent straight or branched hydrocarbon chain wherein 1, 2, or 3 methylene units of the chain are independently and optionally replaced with —O—, —C(O)—, —C(O)O—, —OC(O)—, —N(R)—, —C(O)N(R)—, —(R)NC(O)—, —S—, —SO—, —SO₂—, —C(S)—, or -Cy-;
each R⁴ is independently hydrogen, deuterium, halogen, —CN, —OR⁶, or C₁₋₄ alkyl, or two R⁴ groups on the same carbon are optionally taken together to form ═NR⁶, ═NOR⁶, —O, or ═S;
each R⁵ is independently R, halogen, —CN, —OR, —N(R)₂, —NO₂, —N₃, —SR, or -L¹-R⁶, or two R⁵ groups on the same saturated carbon atom are optionally taken together to form ═NR, ═NOR, ═O, ═S, or a spirocyclic 3-6 membered carbocyclic ring;
each R⁶ is independently hydrogen or C₁₋₆ alkyl optionally substituted with 1, 2, 3, 4, 5, or 6 deuterium or halogen atoms;
m is 0, 1, 2, 3, or 4;
n is 0, 1, 2, 3, or 4;
p is 0, 1, 2, 3, or 4; and
wherein -L- is attached via a covalent bond to each of [symbol] and [symbol] at a carbon, nitrogen, or oxygen atom, and each of the covalent bonds independently replaces a hydrogen on the carbon, nitrogen, or oxygen atom.

In one aspect, the present invention provides a compound of formula I or a pharmaceutically acceptable salt thereof, wherein is a CXCR4 inhibitor selected from compounds disclosed in WO 2017/223229, which [symbol] is incorporated by reference herein.

In some embodiments, the present invention provides a compound of formula I or a pharmaceutically acceptable salt thereof, wherein [symbol] is a CXCR4 inhibitor of Formula B-b:

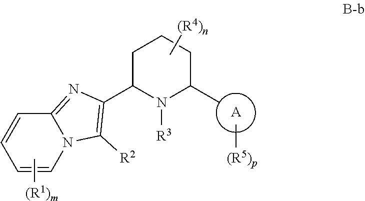

or a pharmaceutically acceptable salt thereof, wherein:
Ring A is a 3-8 membered saturated or partially unsaturated monocyclic carbocyclic ring, phenyl, an 8-10 membered bicyclic aromatic carbocyclic ring, a 4-8 membered saturated or partially unsaturated monocyclic heterocyclic ring having 1-2 heteroatoms independently selected from nitrogen, oxygen, or sulfur, a 5-6 membered monocyclic heteroaromatic ring having 1-4 heteroatoms independently selected from nitrogen, oxygen, or sulfur, or an 8-10 membered bicyclic heteroaromatic ring having 1-5 heteroatoms independently selected from nitrogen, oxygen, or sulfur;
each R¹ is independently —R, halogen, —CN, —OR, —N(R)₂, —NO₂, —N₃, —SR, or -L¹-R⁶;
each R is independently hydrogen or an optionally substituted group selected from C₁₋₆ aliphatic, a 3-8 membered saturated or partially unsaturated monocyclic carbocyclic ring, phenyl, an 8-10 membered bicyclic aromatic carbocyclic ring, a 4-8 membered saturated or partially unsaturated monocyclic heterocyclic ring having 1-2 heteroatoms independently selected from nitrogen, oxygen, or sulfur, a 5-6 membered monocyclic heteroaromatic ring having 1-4 heteroatoms independently selected from nitrogen, oxygen, or sulfur, or an 8-10 membered bicyclic heteroaromatic ring having 1-5 heteroatoms independently selected from nitrogen, oxygen, or sulfur;
each L¹ and L² is independently a covalent bond or a C₁₋₈ bivalent straight or branched hydrocarbon chain wherein 1, 2, or 3 methylene units of the chain are independently and optionally replaced with —O—, —C(O)—, —C(O)O—, —OC(O)—, —N(R)—, —C(O)N(R)—, —(R)NC(O)—, —OC(O)N(R)—, —(R)NC(O)O—, —N(R)C(O)N(R)—, —S—, —SO—, —SO₂—, —SO₂N(R)—, —(R)NSO₂—, —C(S)—, —C(S)O—, —OC(S)—, —C(S) N(R)—, —(R)NC(S)—, —(R)NC(S) N(R)—, or -Cy-;
each -Cy- is independently a bivalent optionally substituted 3-8 membered saturated or partially unsaturated monocyclic carbocyclic ring, optionally substituted phenylene, an optionally substituted 4-8 membered saturated or partially unsaturated monocyclic heterocyclic ring having 1-3 heteroatoms independently selected from nitrogen, oxygen, or sulfur, an optionally substituted 5-6 membered monocyclic heteroaromatic ring having 1-4 heteroatoms independently selected from nitrogen, oxygen, or sulfur, an optionally substituted 8-10 membered bicyclic or bridged bicyclic saturated or partially unsaturated heterocyclic ring having 1-5 heteroatoms independently selected from nitrogen, oxygen, or sulfur, or an optionally substituted 8-10 membered bicyclic or bridged bicyclic heteroaromatic ring having 1-5 heteroatoms independently selected from nitrogen, oxygen, or sulfur;
R² is hydrogen, halogen, —CN, —OR, —N(R)₂, —NO₂, —N₃, —SR, -L²-R⁶, or optionally substituted C₁₋₈ aliphatic;
R³ is hydrogen, optionally substituted C₁₋₆ aliphatic, or -L³-R⁶;
L³ is a C₁₋₆ bivalent straight or branched hydrocarbon chain wherein 1, 2, or 3 methylene units of the chain are independently and optionally replaced with —O—, —C(O)—, —C(O)O—, —OC(O)—, —N(R)—, —C(O)N(R)—, —(R)NC(O)—, —S—, —SO—, —SO₂—, —C(S)—, or -Cy-;
each R⁴ is independently hydrogen, deuterium, halogen, —CN, —OR₆, or C₁₋₄ alkyl, or two R⁴ groups on the same carbon are optionally taken together to form ═NR⁶, ═NOR⁶, —O, or ═S;
each R⁵ is independently R, halogen, —CN, —OR, —N(R)₂, —NO₂, —N₃, —SR, or -L¹-R⁶, or two R⁵ groups on the same saturated carbon atom are optionally taken together to form ═NR, ═NOR, ═O, ═S, or a spirocyclic 3-6 membered carbocyclic ring;
each R⁶ is independently hydrogen or C₁₋₆ alkyl optionally substituted with 1, 2, 3, 4, 5, or 6 deuterium or halogen atoms;
m is 0, 1, 2, 3, or 4;
n is 0, 1, 2, 3, or 4;
p is 0, 1, 2, 3, or 4; and
wherein -L- is attached via a covalent bond to each of [symbol] and [symbol] at a carbon, nitrogen, or oxygen atom, and each of the covalent bonds independently replaces a hydrogen on the carbon, nitrogen, or oxygen atom.

In some embodiments, the present invention provides a compound of formula I:

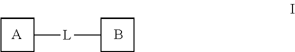

or a pharmaceutically acceptable salt thereof, wherein [symbol] is a CXCR4 inhibitor selected from compounds disclosed in WO 2017/223239, which is incorporated by reference herein.

In one aspect, the present invention provides a compound of formula I or a pharmaceutically acceptable salt thereof, wherein [symbol] is a CXCR4 inhibitor of Formula B-c:

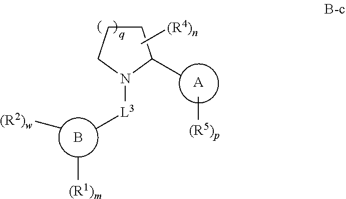

or a pharmaceutically acceptable salt thereof, wherein:
Ring A is a 3-8 membered saturated or partially unsaturated monocyclic carbocyclic ring, phenyl, an 8-10 membered bicyclic aromatic carbocyclic ring, a 4-8 membered saturated or partially unsaturated monocyclic heterocyclic ring having 1-2 heteroatoms independently selected from nitrogen, oxygen, or sulfur, a 5-6 membered monocyclic heteroaromatic ring having 1-4 heteroatoms independently selected from nitrogen, oxygen, or sulfur, or an 8-10 membered bicyclic heteroaromatic ring having 1-5 heteroatoms independently selected from nitrogen, oxygen, or sulfur;
Ring B is selected from

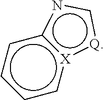

or a 5- or 6-membered aromatic ring having 0-3 heteroatoms selected from nitrogen, oxygen, or sulfur;
X is C or N;
Q is C, N, or S; with the provisos that X and Q are not simultaneously N; and X is C when Q is S;
each R¹ is independently R, halogen, —CN, —OR, —N(R)₂, —NO₂, —N₃, —SR, or -L¹-R⁶;
each R is independently hydrogen or an optionally substituted group selected from C₁₋₆ aliphatic, a 3-8 membered saturated or partially unsaturated monocyclic carbocyclic ring, phenyl, an 8-10 membered bicyclic aromatic carbocyclic ring, a 4-8 membered saturated or partially unsaturated monocyclic heterocyclic ring having 1-2 heteroatoms independently selected from nitrogen, oxygen, or sulfur, a 5-6 membered monocyclic heteroaromatic ring having 1-4 heteroatoms independently selected from nitrogen, oxygen, or sulfur, or an 8-10 membered bicyclic heteroaromatic ring having 1-5 heteroatoms independently selected from nitrogen, oxygen, or sulfur;
each L¹ and L² is independently a covalent bond or a C₁₋₈ bivalent straight or branched hydrocarbon chain wherein 1, 2, or 3 methylene units of the chain are independently and optionally replaced with —O—, —C(O)—, —C(O)O—, —OC(O)—, —N(R)—, —C(O)N(R)—, —(R)NC(O)—, —OC(O)N(R)—, —(R)NC(O)O—, —N(R)C(O)N(R)—, —S—, —SO—, —SO₂—, —SO₂N(R)—, —(R)NSO₂—, —C(S)—, —C(S)O—, —OC(S)—, —C(S) N(R)—, —(R)NC(S)—, —(R)NC(S) N(R)—, or -Cy-;
each -Cy- is independently a bivalent optionally substituted 3-8 membered saturated or partially unsaturated monocyclic carbocyclic ring, optionally substituted phenylene, an optionally substituted 4-8 membered saturated or partially unsaturated monocyclic heterocyclic ring having 1-3 heteroatoms independently selected from nitrogen, oxygen, or sulfur, an optionally substituted 5-6 membered monocyclic heteroaromatic ring having 1-4 heteroatoms independently selected from nitrogen, oxygen, or sulfur, an optionally substituted 8-10 membered bicyclic or bridged bicyclic saturated or partially unsaturated heterocyclic ring having 1-5 heteroatoms independently selected from nitrogen, oxygen, or sulfur, or an optionally substituted 8-10 membered bicyclic or bridged bicyclic heteroaromatic ring having 1-5 heteroatoms independently selected from nitrogen, oxygen, or sulfur;
R² is hydrogen, -L²-R⁶, or optionally substituted C₁₋₈ aliphatic;
L³ is a C₁₋₄ bivalent straight chain hydrocarbon substituted with 1, 2, 3, or 4 independently selected R³ groups;
each R³ is independently hydrogen, deuterium, halogen, —OR₆, or optionally substituted C₁₋₃ aliphatic; or two R³ groups on the same saturated carbon atom are optionally taken together to form ═O or ═S;
each R⁴ is independently hydrogen, deuterium, halogen, —CN, —OR₆, or C₁₋₄ alkyl, or two R⁴ groups on the same carbon are optionally taken together to form ═NR⁶, —NOR⁶, —O, or ═S;
each R⁵ is independently R, halogen, —CN, —OR, —N(R)₂, —NO₂, —N₃, —SR, or -L¹-R⁶, or two R⁵ groups on the same saturated carbon atom are optionally taken together to form ═NR, =NOR, ═O, ═S, or a spirocyclic 3-6 membered carbocyclic ring;
each R⁶ is independently hydrogen or C₁₋₆ alkyl optionally substituted with 1, 2, 3, 4, 5, or 6 deuterium or halogen atoms;
m is 0, 1, 2, 3, or 4;
n is 0, 1, 2, 3, or 4;
p is 0, 1, 2, 3, or 4;
q is 0, 1, 2, or 3;
w is 0 or 1; and
wherein -L- is attached via a covalent bond to each of [symbol] and [symbol] at a carbon, nitrogen, or oxygen atom, and each of the covalent bonds independently replaces a hydrogen on the carbon, nitrogen, or oxygen atom.

In some embodiments, the present invention provides a compound of formula I:

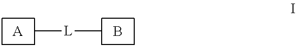

or a pharmaceutically acceptable salt thereof, wherein [symbol] is a CXCR4 inhibitor selected from compounds disclosed in WO 2017/223243, which is incorporated by reference herein.

In some embodiments, the present invention provides a compound of formula I or a pharmaceutically acceptable salt thereof, wherein [symbol] is a CXCR4 inhibitor of Formula B-d:

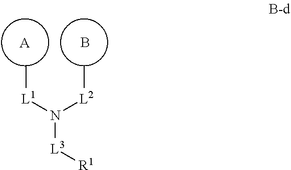

or a pharmaceutically acceptable salt thereof, wherein:
Ring A is an optionally substituted ring selected from a 5-6 membered monocyclic heteroaromatic ring having 1-4 heteroatoms independently selected from nitrogen, oxygen, or sulfur, or an 8-10 membered bicyclic partially unsaturated or aromatic ring having 0-3 heteroatoms independently selected from nitrogen, oxygen, or sulfur;
Ring B is an optionally substituted ring selected from a 5-6 membered monocyclic heteroaromatic ring having 1-4 heteroatoms independently selected from nitrogen, oxygen, or sulfur, or an 8-10 membered bicyclic partially unsaturated or aromatic ring having 0-3 heteroatoms independently selected from nitrogen, oxygen, or sulfur;
L¹ is —CH₂— or —CH(CH₃)—;
L² is a covalent bond, —CH₂—, or —CH(CH₃)—;
L³ is a C₂₋₃ bivalent straight or branched hydrocarbon chain;
R¹ is —Cy, —OR, —N(R)₂, —C(O)N(R)₂, or —N(R)C(O)R;
each R is independently hydrogen or an optionally substituted group selected from C₁₋₆ aliphatic, a 3-8 membered saturated or partially unsaturated monocyclic carbocyclic ring, phenyl, an 8-10 membered bicyclic aromatic carbocyclic ring, a 4-8 membered saturated or partially unsaturated monocyclic heterocyclic ring having 1-2 heteroatoms independently selected from nitrogen, oxygen, or sulfur, a 5-6 membered monocyclic heteroaromatic ring having 1-4 heteroatoms independently selected from nitrogen, oxygen, or sulfur, or an 8-10 membered bicyclic heteroaromatic ring having 1-5 heteroatoms independently selected from nitrogen, oxygen, or sulfur, or two R groups on the same nitrogen are optionally take together with their intervening atoms to form a 5-6 membered saturated, partially unsaturated, or aromatic heterocyclic ring having 1-2 heteroatoms in addition to the nitrogen attached thereto independently selected from nitrogen, oxygen, or sulfur;
-Cy is an optionally substituted ring selected from a 3-8 membered saturated or partially unsaturated monocyclic carbocyclic ring, phenyl, an 8-10 membered bicyclic aromatic carbocyclic ring, a 4-8 membered saturated or partially unsaturated monocyclic heterocyclic ring having 1-2 heteroatoms independently selected from nitrogen, oxygen, or sulfur, a 5-6 membered monocyclic heteroaromatic ring having 1-4 heteroatoms independently selected from nitrogen, oxygen, or sulfur, or an 8-10 membered bicyclic heteroaromatic ring having 1-5 heteroatoms independently selected from nitrogen, oxygen, or sulfur; and
wherein -L- is attached via a covalent bond to each of [symbol] and [symbol] at a carbon, nitrogen, or oxygen atom, and each of the covalent bonds independently replaces a hydrogen on the carbon, nitrogen, or oxygen atom.

In some embodiments, the present invention provides a compound of formula I or a pharmaceutically acceptable salt thereof, wherein [symbol] is a CXCR4 inhibitor selected from compounds disclosed in WO 2019/126106, which is incorporated by reference herein.

In some embodiments, the present invention provides a compound of formula I or a pharmaceutically acceptable salt thereof, wherein [symbol] is a CXCR4 inhibitor of Formula B-e:

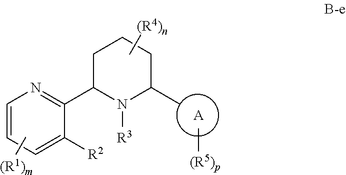

or a pharmaceutically acceptable salt thereof, wherein:
Ring A is a 3-8 membered saturated or partially unsaturated monocyclic carbocyclic ring, phenyl, an 8-10 membered bicyclic aromatic carbocyclic ring, a 4-8 membered saturated or partially unsaturated monocyclic heterocyclic ring having 1-2 heteroatoms independently selected from nitrogen, oxygen, or sulfur, a 5-6 membered monocyclic heteroaromatic ring having 1-4 heteroatoms independently selected from nitrogen, oxygen, or sulfur, or an 8-10 membered bicyclic heteroaromatic ring having 1-5 heteroatoms independently selected from nitrogen, oxygen, or sulfur;
each R¹ is independently R, halogen, —CN, —OR, —N(R)₂, —NO₂, —N₃, —SR, or -L¹-R⁶;
R² is —CN, —OR, —N(R)₂, —SR, -L²-R⁶, or optionally substituted C₁₋₈ aliphatic;
R³ is hydrogen, optionally substituted C₁₋₆ aliphatic, or -L³-R⁶;
each —R is independently hydrogen or an optionally substituted group selected from C₁₋₆ aliphatic, a 3-8 membered saturated or partially unsaturated monocyclic carbocyclic ring, phenyl, an 8-10 membered bicyclic aromatic carbocyclic ring, a 4-8 membered saturated or partially unsaturated monocyclic heterocyclic ring having 1-2 heteroatoms independently selected from nitrogen, oxygen, or sulfur, a 5-6 membered monocyclic heteroaromatic ring having 1-4 heteroatoms independently selected from nitrogen, oxygen, or sulfur, or an 8-10 membered bicyclic heteroaromatic ring having 1-5 heteroatoms independently selected from nitrogen, oxygen, or sulfur;
each L¹ and L² is independently a covalent bond or a C₁₋₈ bivalent straight or branched, optionally substituted hydrocarbon chain wherein 1, 2, or 3 methylene units of the chain are independently and optionally replaced with —O—, —C(O)—, —C(O)O—, —OC(O)—, —N(R)—, —C(O)N(R)—, —(R)NC(O)—, —OC(O)N(R)—, —(R)NC(O)O—, —N(R)C(O)N(R)—, —S—, —SO—, —SO₂—, —SO₂N(R)—, —(R)NSO₂—, —C(S)—, —C(S)O—, —OC(S)—, —C(S) N(R)—, —(R)NC(S)—, —(R)NC(S) N(R)—, or -Cy-;
each -Cy- is independently a bivalent optionally substituted 3-8 membered saturated or partially unsaturated monocyclic carbocyclic ring, optionally substituted phenylene, an optionally substituted 3-8 membered saturated or partially unsaturated monocyclic heterocyclic ring having 1-3 heteroatoms independently selected from nitrogen, oxygen, or sulfur, an optionally substituted 5-6 membered monocyclic heteroaromatic ring having 1-4 heteroatoms independently selected from nitrogen, oxygen, or sulfur, an optionally substituted 8-10 membered bicyclic or bridged bicyclic saturated or partially unsaturated heterocyclic ring having 1-5 heteroatoms independently selected from nitrogen, oxygen, or sulfur, or an optionally substituted 8-10 membered bicyclic or bridged bicyclic heteroaromatic ring having 1-5 heteroatoms independently selected from nitrogen, oxygen, or sulfur;
L³ is a C₁₋₆ bivalent straight or branched hydrocarbon chain wherein 1, 2, or 3 methylene units of the chain are independently and optionally replaced with —O—, —C(O)—, —C(O)O—, —OC(O)—, —N(R)—, —C(O)N(R)—, —(R)NC(O)—, —S—, —SO—, —SO₂—, —C(S)—, or -Cy-;
each R⁴ is independently hydrogen, deuterium, halogen, —CN, —OR₆, or C₁₋₄ alkyl, or two R⁴ groups on the same carbon are optionally taken together to form ═NR⁶, =NOR⁶, =O, or ═S;
each R⁵ is independently R, halogen, —CN, —OR, —N(R)₂, —NO₂, —N₃, —SR, or -L¹-R⁶, or two R⁵ groups on the same saturated carbon atom are optionally taken together to form ═NR, =NOR, ═O, ═S, or a spirocyclic 3-6 membered carbocyclic ring;
each R⁶ is independently hydrogen or C₁₋₆ alkyl optionally substituted with 1, 2, 3, 4, 5, or 6 deuterium or halogen atoms;
m is 0, 1, 2, or 3;
n is 0, 1, 2, 3, or 4;
p is 0, 1, 2, 3, or 4; provided that

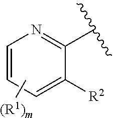

is not the same as

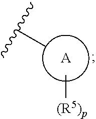

and
wherein -L- is attached via a covalent bond to each of [symbol] and [symbol] at a carbon, nitrogen, or oxygen atom, and each of the covalent bonds independently replaces a hydrogen on the carbon, nitrogen, or oxygen atom.

In some embodiments, the present invention provides a compound of formula I or a pharmaceutically acceptable salt thereof, wherein [symbol] is a CXCR4 inhibitor selected from compounds disclosed in WO 2020/264292, which is incorporated by reference herein.

In some embodiments, the present invention provides a compound of formula I or a pharmaceutically acceptable salt thereof, wherein [symbol] is a CXCR4 inhibitor of Formula B-f:

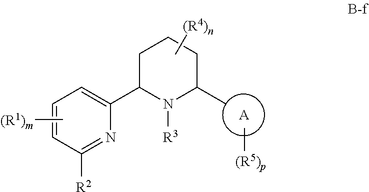

or a pharmaceutically acceptable salt thereof, wherein:
Ring A is a 3-8 membered saturated or partially unsaturated monocyclic carbocyclic ring, phenyl, an 8-10 membered bicyclic aromatic carbocyclic ring, a 4-8 membered saturated or partially unsaturated monocyclic heterocyclic ring having 1-2 heteroatoms independently selected from nitrogen, oxygen, or sulfur, a 5-6 membered monocyclic heteroaromatic ring having 1-4 heteroatoms independently selected from nitrogen, oxygen, or sulfur, or an 8-10 membered bicyclic heteroaromatic ring having 1-5 heteroatoms independently selected from nitrogen, oxygen, or sulfur;
each R¹ is independently R, halogen, —CN, —OR, —N(R)₂, —NO₂, —N₃, —SR, or -L¹-R⁶,
each —R is independently hydrogen or an optionally substituted group selected from C₁₋₆ aliphatic, a 3-8 membered saturated or partially unsaturated monocyclic carbocyclic ring, phenyl, an 8-10 membered bicyclic aromatic carbocyclic ring, a 4-8 membered saturated or partially unsaturated monocyclic heterocyclic ring having 1-2 heteroatoms independently selected from nitrogen, oxygen, or sulfur, a 5-6 membered monocyclic heteroaromatic ring having 1-4 heteroatoms independently selected from nitrogen, oxygen, or sulfur, or an 8-10 membered bicyclic heteroaromatic ring having 1-5 heteroatoms independently selected from nitrogen, oxygen, or sulfur;
each L¹ is independently a covalent bond or a C₁₋₆ bivalent straight or branched, optionally substituted hydrocarbon chain wherein 1, 2, or 3 methylene units of the chain are independently and optionally replaced with —O—, —C(O)—, —C(O)O—, —OC(O)—, —N(R)—, —C(O)N(R)—, —(R)NC(O)—, —OC(O)N(R)—, —(R)NC(O)O—, —N(R)C(O)N(R)—, —S—, —SO—, —SO₂—, —SO₂N(R)—, —(R)NSO₂—, —C(S)—, —C(S)O—, —OC(S)—, —C(S) N(R)—, —(R)NC(S)—, —(R)NC(S) N(R)—, or -Cy-;
each -Cy- is independently a bivalent optionally substituted 3-8 membered saturated or partially unsaturated monocyclic carbocyclic ring, optionally substituted phenylene, an optionally substituted 3-8 membered saturated or partially unsaturated monocyclic heterocyclic ring having 1-3 heteroatoms independently selected from nitrogen, oxygen, or sulfur, an optionally substituted 5-6 membered monocyclic heteroaromatic ring having 1-4 heteroatoms independently selected from nitrogen, oxygen, or sulfur, an optionally substituted 8-10 membered bicyclic or bridged bicyclic saturated or partially unsaturated heterocyclic ring having 1-5 heteroatoms independently selected from nitrogen, oxygen, or sulfur, or an optionally substituted 8-10 membered bicyclic or bridged bicyclic heteroaromatic ring having 1-5 heteroatoms independently selected from nitrogen, oxygen, or sulfur;
R² is

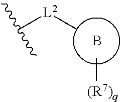

or a C₁₋₆ aliphatic group substituted with one-CN, —N(R)₂, or —C(O)N(R)₂ group and wherein 1 or 2 methylene units of the aliphatic group are independently and optionally replaced with —O—, —C(O)—, —C(O)O—, —OC(O)—, —N(R)—, —S—, —SO—, or —SO₂—;
L² is a covalent bond or a C₁₋₄ bivalent straight or branched, optionally substituted hydrocarbon chain wherein 1 or 2 methylene units of the chain are independently and optionally replaced with —O—, —C(O)—, —C(O)O—, —OC(O)—, —N(R)—, —C(O)N(R)—, —(R)NC(O)—, —S—, —SO—, —SO₂—, —SO₂N(R)—, —(R)NSO₂—, - or C(S)—;
Ring B is phenyl, an 8-10 membered bicyclic aromatic carbocyclic ring, an 8-10 membered partially unsaturated carbocyclic ring, a 5-6 membered monocyclic heteroaromatic ring having 1-4 heteroatoms independently selected from nitrogen, oxygen, or sulfur, an 8-10 membered bicyclic heteroaromatic ring having 1-5 heteroatoms independently selected from nitrogen, oxygen, or sulfur, or an 8-10 membered bicyclic partially unsaturated heterocyclic ring having 1-5 heteroatoms independently selected from nitrogen, oxygen, or sulfur;
R³ is hydrogen or C₁₋₃ aliphatic optionally substituted with 1, 2 or 3 substituents independently selected from deuterium or halogen;
each R⁴ is independently hydrogen, deuterium, halogen, —CN, —OR₆, or C₁₋₄ alkyl, or two R⁴ groups on the same carbon are optionally taken together to form ═NR⁶, =NOR⁶, ═O, or ═S;
each R⁵ is independently R, halogen, —CN, —OR, —N(R)₂, —NO₂, —N₃, —SR, or -L¹-R⁶, or two R⁵ groups on the same carbon atom are optionally taken together to form ═NR, =NOR, ═O, ═S, or a spirocyclic 3-6 membered carbocyclic ring;
each R⁶ is independently hydrogen or C₁₋₆ alkyl optionally substituted with 1, 2, 3, 4, 5, or 6 deuterium or halogen atoms;
each R⁷ is independently R, halogen, —CN, —OR, —N(R)₂, —NO₂, —N₃, —SR, or -L¹-R⁶, or two R⁷ groups on the same carbon atom are optionally taken together to form ═O or ═S;
m is 0, 1, 2, or 3;
n is 0, 1, 2, 3, or 4;
p is 0, 1, 2, 3, or 4;
q is 0, 1, 2, or 3;
provided that

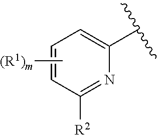

is not the same as

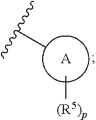

and
wherein -L- is attached via a covalent bond to each of [symbol] and [symbol] at a carbon, nitrogen, or oxygen atom, and each of the covalent bonds independently replaces a hydrogen on the carbon, nitrogen, or oxygen atom.

In some embodiments, the present invention provides a compound of formula I or a pharmaceutically acceptable salt thereof, wherein [symbol] is a CXCR4 inhibitor selected from compounds disclosed in WO 2021/263203, which is incorporated by reference herein.

In one aspect, the present invention provides a compound of formula II:

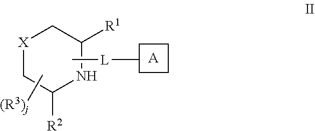

or a pharmaceutically acceptable salt, solvate, or hydrate thereof, wherein:
—X— is carbon, nitrogen, oxygen, sulfur, SO, or SO₂;
-L- is a covalent bond or a C₁₋₈ bivalent straight or branched, saturated or unsaturated hydrocarbon chain wherein 1-4 methylene units of the chain are independently and optionally replaced with —O—, —C(O)—, —C(S)—, -Cy-, —C(R^{A})₂—, —CH(R^{A})—, —CH(OR)—, —N(R)—, —S—, —S(O)—, or —S(O)₂—;
each R^{A} is independently hydrogen, halogen, —CN, optionally substituted group selected from C₁₋₆ aliphatic; phenyl; an 8-10 membered bicyclic aryl ring, a 3-7 membered saturated or partially unsaturated monocyclic carbocyclic ring; a 4-8 membered saturated or partially unsaturated monocyclic heterocyclic ring having 1-2 heteroatoms independently selected from nitrogen, oxygen, and sulfur; a 5-6 membered monocyclic heteroaryl ring having 1-4 heteroatoms independently selected from nitrogen, oxygen, and sulfur; and an 8-10 membered bicyclic heteroaryl ring having 1-5 heteroatoms independently selected from nitrogen, oxygen, and sulfur; or
   two R^{A} groups on the same carbon are optionally taken together, with the carbon they are attached to, to form: an optionally substituted 3-10 membered monocyclic or bicyclic saturated, or partially unsaturated, carbocyclic ring; or an optionally substituted 3-10 membered monocyclic or bicyclic saturated, or partially unsaturated, heterocyclic ring, having 0-3 heteroatoms independently selected from nitrogen, oxygen, and sulfur;
each R is independently hydrogen, or an optionally substituted group selected from C₁₋₆ aliphatic; phenyl; an 8-10 membered bicyclic aryl ring, a 3-7 membered saturated or partially unsaturated monocyclic carbocyclic ring; a 4-8 membered saturated or partially unsaturated monocyclic heterocyclic ring having 1-2 heteroatoms independently selected from nitrogen, oxygen, and sulfur; a 5-6 membered monocyclic heteroaryl ring having 1-4 heteroatoms independently selected from nitrogen, oxygen, and sulfur; and an 8-10 membered bicyclic heteroaryl ring having 1-5 heteroatoms independently selected from nitrogen, oxygen, and sulfur; or
   two R groups on the same nitrogen are optionally taken together with the nitrogen to form an optionally substituted 4-7 membered monocyclic saturated, partially unsaturated, or heteroaryl ring having, in addition to the nitrogen, 0-3 heteroatoms independently selected from nitrogen, oxygen, and sulfur;
each -Cy- is independently an optionally substituted bivalent ring selected from a 4-7 membered saturated or partially unsaturated heterocyclylenyl having 1-2 heteroatoms independently selected from nitrogen, oxygen, and sulfur, phenylenyl, a 3-7 membered saturated or partially unsaturated carbocyclylenyl, or a 5-6 membered heteroarylenyl having 1-4 heteroatoms independently selected from nitrogen, oxygen, and sulfur;
each of R¹ and R² is independently —C(O)N(R″) 2; —C(O) OR″; C₁₋₇ aliphatic; phenyl; a 5-6 membered monocyclic heteroaryl ring having 1-4 heteroatoms independently selected from nitrogen, oxygen, and sulfur; a 3-7 membered saturated or partially unsaturated monocyclic carbocyclic ring; a 4-8 membered saturated or partially unsaturated monocyclic heterocyclic ring having 1-2 heteroatoms independently selected from nitrogen, oxygen, and sulfur; a 5-10 membered saturated or partially unsaturated bridged bicyclic ring having 0-3 heteroatoms independently selected from nitrogen, oxygen, and sulfur; a 5-10 membered saturated or partially unsaturated spirocyclic ring having 0-3 heteroatoms independently selected from nitrogen, oxygen, and sulfur; or an 8-10 membered partially aromatic or heteroaromatic bicyclic heterocyclic ring having 1-4 heteroatoms independently selected from nitrogen, oxygen, and sulfur; each of which is substituted with m instances of R′;
each occurrence of R′ is independently hydrogen, halogen, or a C₁₋₆ aliphatic group optionally substituted with 1-5 halogens;
each occurrence of R″ is independently hydrogen, or a C₁₋₆ aliphatic group optionally substituted with 1-5 halogens;
each R³ is independently —N(R)₂, —OR, or C₁₋₇ aliphatic optionally substituted with 1-5 groups independently selected from halogen, —N(R)₂, and —OR, and wherein 1 or 2 methylene units of the aliphatic group are optionally replaced by —N(R)— or a methyl group is optionally replaced by —N(R)₂;
[symbol] is selected from the following:

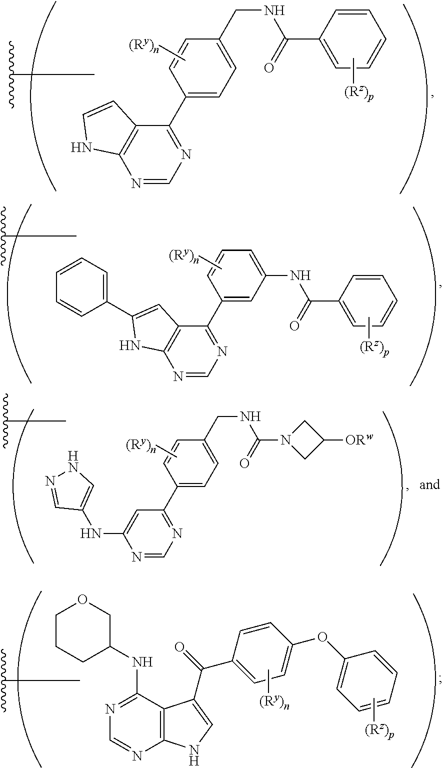

R^{w} is hydrogen, or C₁₋₇ aliphatic optionally substituted with 1-5 halogens; R^{y} is hydrogen, halogen, or C₁₋₇ aliphatic optionally substituted with 1-5 halogens; R^{z} is hydrogen, halogen, or C₁₋₇ aliphatic optionally substituted with 1-5 halogens;
j is 0, 1, or 2;
m is 0, 1, 2, 3, or 4;
n is 0, 1, 2, 3, or 4;
p is 0, 1, 2, 3, 4, or 5; and
wherein -L- is attached via a covalent bond to each of [symbol] and

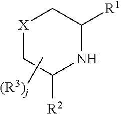

at a carbon, nitrogen, or oxygen atom, and each covalent bond independently replaces a hydrogen on the carbon, nitrogen, or oxygen atom.

As defined generally above, —X— is carbon, nitrogen, oxygen, or sulfur.

In some embodiments, —X— is carbon. In some embodiments, —X— is nitrogen. In some embodiments, —X— is oxygen. In some embodiments, —X— is sulfur.

In some embodiments, —X— is selected from those depicted in Table 1, below.

As defined generally above, -L- is one of the following: (a) C₁₋₈ bivalent straight or branched, saturated or unsaturated hydrocarbon chain wherein 1-4 methylene units of the chain are independently and optionally replaced with —O—, —C(O)—, —C(S)—, -Cy-, —C(R^{A})₂—, —CH(R^{A})—, —CH(OR)—, —N(R)—, —S—, —S(O)—, or —S(O)₂—; or (b) a covalent bond.

In some embodiments, -L- is C₁₋₈ bivalent straight or branched, saturated or unsaturated hydrocarbon chain wherein 1-4 methylene units of the chain are independently and optionally replaced with —O—, —C(O)—, —C(S)—, -Cy-, —C(R^{A})₂—, —CH(R^{A})—, —CH(OR)—, —N(R)—, —S—, —S(O)—, or —S(O)₂; or a covalent bond.

In some embodiments, -L- is C₁₋₈ bivalent straight or branched, saturated or unsaturated hydrocarbon chain wherein 1-4 methylene units of the chain are independently and optionally replaced with —O—. In some embodiments, -L- is C₁₋₈ bivalent straight or branched, saturated or unsaturated hydrocarbon chain wherein 1-4 methylene units of the chain are independently and optionally replaced with —C(O)—. In some embodiments, -L- is C₁₋₈ bivalent straight or branched, saturated or unsaturated hydrocarbon chain wherein 1-4 methylene units of the chain are independently and optionally replaced with —C(S)—. In some embodiments, -L- is C₁₋₈ bivalent straight or branched, saturated or unsaturated hydrocarbon chain wherein 1-4 methylene units of the chain are independently and optionally replaced with -Cy-. In some embodiments, -L- is C₁₋₈ bivalent straight or branched, saturated or unsaturated hydrocarbon chain wherein 1-4 methylene units of the chain are independently and optionally replaced with —C(R^{A})₂—. In some embodiments, -L- is C₁₋₈ bivalent straight or branched, saturated or unsaturated hydrocarbon chain wherein 1-4 methylene units of the chain are independently and optionally replaced with —CH(R^{A})—. In some embodiments, -L- is C₁₋₈ bivalent straight or branched, saturated or unsaturated hydrocarbon chain wherein 1-4 methylene units of the chain are independently and optionally replaced with —CH(OR)—. In some embodiments, -L- is C₁₋₈ bivalent straight or branched, saturated or unsaturated hydrocarbon chain wherein 1-4 methylene units of the chain are independently and optionally replaced with —N(R)—. In some embodiments, -L- is C₁₋₈ bivalent straight or branched, saturated or unsaturated hydrocarbon chain wherein 1-4 methylene units of the chain are independently and optionally replaced with —S—. In some embodiments, -L- is C₁₋₈ bivalent straight or branched, saturated or unsaturated hydrocarbon chain wherein 1-4 methylene units of the chain are independently and optionally replaced with —S(O)—. In some embodiments, -L- is C₁₋₈ bivalent straight or branched, saturated or unsaturated hydrocarbon chain wherein 1-4 methylene units of the chain are independently and optionally replaced with —S(O)₂—.

In some embodiments, -L- is a covalent bond.

In some embodiments, -L- is

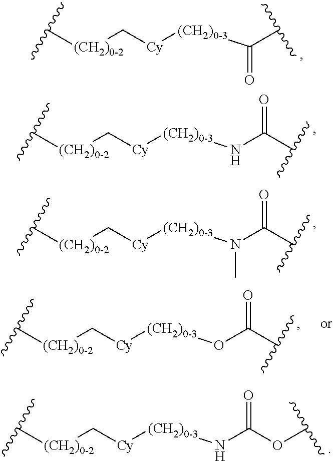

In some embodiments, -L- is

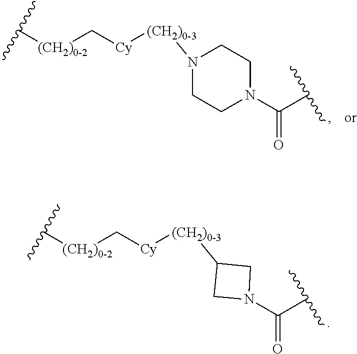

In some embodiments, -L- is

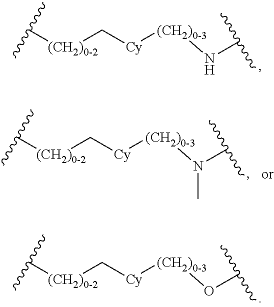

In some embodiments, -L- is

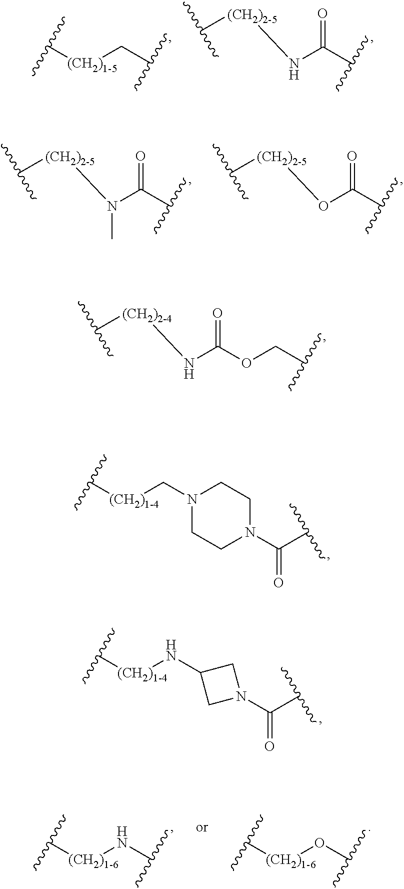

In some embodiments, -L- is selected from those depicted in Table 1, below.

As defined generally above, each R^{A} is independently hydrogen, halogen, —CN, optionally substituted group selected from C₁₋₆ aliphatic; phenyl; an 8-10 membered bicyclic aryl ring, a 3-7 membered saturated or partially unsaturated monocyclic carbocyclic ring; a 4-8 membered saturated or partially unsaturated monocyclic heterocyclic ring having 1-2 heteroatoms independently selected from nitrogen, oxygen, and sulfur; a 5-6 membered monocyclic heteroaryl ring having 1-4 heteroatoms independently selected from nitrogen, oxygen, and sulfur; or an 8-10 membered bicyclic heteroaryl ring having 1-5 heteroatoms independently selected from nitrogen, oxygen, and sulfur.

In some embodiments, each R^{A} is independently hydrogen, halogen, —CN, optionally substituted group selected from C₁₋₆ aliphatic; phenyl; an 8-10 membered bicyclic aryl ring, a 3-7 membered saturated or partially unsaturated monocyclic carbocyclic ring; a 4-8 membered saturated or partially unsaturated monocyclic heterocyclic ring having 1-2 heteroatoms independently selected from nitrogen, oxygen, and sulfur; a 5-6 membered monocyclic heteroaryl ring having 1-4 heteroatoms independently selected from nitrogen, oxygen, and sulfur; or an 8-10 membered bicyclic heteroaryl ring having 1-5 heteroatoms independently selected from nitrogen, oxygen, and sulfur.

In some embodiments, each R^{A} is independently halogen. In some embodiments, each R^{A} is independently —CN. In some embodiments, each R^{A} is independently optionally substituted C₁₋₆ aliphatic. In some embodiments, each R^{A} is independently optionally substituted phenyl. In some embodiments, each R^{A} is independently optionally substituted 8-10 membered bicyclic aryl ring. In some embodiments, each R^{A} is independently optionally substituted 3-7 membered saturated or partially unsaturated monocyclic carbocyclic ring. In some embodiments, each R^{A} is independently optionally substituted 4-8 membered saturated or partially unsaturated monocyclic heterocyclic ring having 1-2 heteroatoms independently selected from nitrogen, oxygen, and sulfur. In some embodiments, each R^{A} is independently optionally substituted 5-6 membered monocyclic heteroaryl ring having 1-4 heteroatoms independently selected from nitrogen, oxygen, and sulfur. In some embodiments, each R^{A} is independently optionally substituted 8-10 membered bicyclic heteroaryl ring having 1-5 heteroatoms independently selected from nitrogen, oxygen, and sulfur.

As defined generally above, two R^{A} groups on the same carbon are optionally taken together, with the carbon they are attached to, to form an optionally substituted 3-10 membered monocyclic or bicyclic saturated, or partially unsaturated, carbocyclic ring; or an optionally substituted 3-10 membered monocyclic or bicyclic saturated, or partially unsaturated, heterocyclic ring, having 0-3 heteroatoms independently selected from nitrogen, oxygen, and sulfur.

In some embodiments, two R^{A} groups on the same carbon are optionally taken together, with the carbon they are attached to, to form an optionally substituted 3-10 membered monocyclic or bicyclic saturated, or partially unsaturated, carbocyclic ring; or an optionally substituted 3-10 membered monocyclic or bicyclic saturated, or partially unsaturated, heterocyclic ring, having 0-3 heteroatoms independently selected from nitrogen, oxygen, and sulfur.

In some embodiments, two R^{A} groups on the same carbon are optionally taken together, with the carbon they are attached to, to form an optionally substituted 3-10 membered monocyclic or bicyclic saturated, or partially unsaturated, carbocyclic ring. In some embodiments, two R^{A} groups on the same carbon are optionally taken together, with the carbon they are attached to, to form an optionally substituted 3-10 membered monocyclic or bicyclic saturated, or partially unsaturated, heterocyclic ring, having 0-3 heteroatoms independently selected from nitrogen, oxygen, and sulfur.

In some embodiments, R^{A} is selected from those depicted in Table 1, below.

As defined generally above, each R is independently hydrogen, or an optionally substituted group selected from C₁₋₆ aliphatic; phenyl; an 8-10 membered bicyclic aryl ring, a 3-7 membered saturated or partially unsaturated monocyclic carbocyclic ring; a 4-8 membered saturated or partially unsaturated monocyclic heterocyclic ring having 1-2 heteroatoms independently selected from nitrogen, oxygen, and sulfur; a 5-6 membered monocyclic heteroaryl ring having 1-4 heteroatoms independently selected from nitrogen, oxygen, and sulfur; and an 8-10 membered bicyclic heteroaryl ring having 1-5 heteroatoms independently selected from nitrogen, oxygen, and sulfur.

In some embodiments, R is hydrogen. In some embodiments, R is an optionally substituted C₁₋₆ aliphatic. In some embodiments, R is an optionally substituted phenyl. In some embodiments, R is an optionally substituted 8-10 membered bicyclic aryl ring. a 3-7 membered saturated or partially unsaturated monocyclic carbocyclic ring. In some embodiments, R is an optionally substituted 4-8 membered saturated or partially unsaturated monocyclic heterocyclic ring having 1-2 heteroatoms independently selected from nitrogen, oxygen, and sulfur. In some embodiments, R is an optionally substituted 5-6 membered monocyclic heteroaryl ring having 1-4 heteroatoms independently selected from nitrogen, oxygen, and sulfur. In some embodiments, R is an optionally substituted 8-10 membered bicyclic heteroaryl ring having 1-5 heteroatoms independently selected from nitrogen, oxygen, and sulfur.

As defined generally above, two R groups on the same nitrogen are optionally taken together with the nitrogen to form an optionally substituted 4-7 membered monocyclic saturated, partially unsaturated, or heteroaryl ring having, in addition to the nitrogen, 0-3 heteroatoms independently selected from nitrogen, oxygen, and sulfur.

In some embodiments, two R groups on the same nitrogen are optionally taken together with the nitrogen to form an optionally substituted 4-7 membered monocyclic saturated, partially unsaturated, or heteroaryl ring having, in addition to the nitrogen, 0-3 heteroatoms independently selected from nitrogen, oxygen, and sulfur.

In some embodiments, R is selected from those depicted in Table 1, below.

As defined generally above, each -Cy- is independently an optionally substituted bivalent ring selected from a 4-7 membered saturated or partially unsaturated heterocyclylenyl having 1-2 heteroatoms independently selected from nitrogen, oxygen, and sulfur, phenylenyl, a 3-7 membered saturated or partially unsaturated carbocyclylenyl, or a 5-6 membered heteroarylenyl having 1-4 heteroatoms independently selected from nitrogen, oxygen, and sulfur.

In some embodiments, -Cy- is an optionally substituted bivalent ring selected from a 4-7 membered saturated or partially unsaturated heterocyclylenyl having 1-2 heteroatoms independently selected from nitrogen, oxygen, and sulfur.

In some embodiments, -Cy- is an optionally substituted phenylenyl.

In some embodiments, -Cy- is an optionally substituted 3-7 membered saturated or partially unsaturated carbocyclylenyl. In some embodiments, -Cy- is an optionally substituted 5-6 membered heteroarylenyl having 1-4 heteroatoms independently selected from nitrogen, oxygen, and sulfur.

In some embodiments, -Cy- is selected from those depicted in Table 1, below.

As defined generally above, R¹ is one of the following:
(a) C₁₋₇ aliphatic; phenyl; a 5-6 membered monocyclic heteroaryl ring having 1-4 heteroatoms independently selected from nitrogen, oxygen, and sulfur; a 3-7 membered saturated or partially unsaturated monocyclic carbocyclic ring; a 4-8 membered saturated or partially unsaturated monocyclic heterocyclic ring having 1-2 heteroatoms independently selected from nitrogen, oxygen, and sulfur; a 5-10 membered saturated or partially unsaturated bridged bicyclic ring having 0-3 heteroatoms independently selected from nitrogen, oxygen, and sulfur; a 5-10 membered saturated or partially unsaturated spirocyclic ring having 0-3 heteroatoms independently selected from nitrogen, oxygen, and sulfur; or an 8-10 membered partially aromatic or heteroaromatic bicyclic heterocyclic ring having 1-4 heteroatoms independently selected from nitrogen, oxygen, and sulfur; each of which is substituted with m instances of R′; or
(b) —C(O)N(R″) 2, or —C(O) OR″.

In some embodiments, R¹ is C₁₋₇ aliphatic, which is substituted with m instances of R′. In some embodiments, R¹ is phenyl, which is substituted with m instances of R′. In some embodiments, R¹ is a 5-6 membered monocyclic heteroaryl ring having 1-4 heteroatoms independently selected from nitrogen, oxygen, and sulfur, which is substituted with m instances of R′. In some embodiments, R¹ is a 3-7 membered saturated or partially unsaturated monocyclic carbocyclic ring, which is substituted with m instances of R′. In some embodiments, R¹ is a 4-8 membered saturated or partially unsaturated monocyclic heterocyclic ring having 1-2 heteroatoms independently selected from nitrogen, oxygen, and sulfur, which is substituted with m instances of R′. In some embodiments, R¹ is a 5-10 membered saturated or partially unsaturated bridged bicyclic ring having 0-3 heteroatoms independently selected from nitrogen, oxygen, and sulfur, which is substituted with m instances of R′. In some embodiments, R¹ is a 5-10 membered saturated or partially unsaturated spirocyclic ring having 0-3 heteroatoms independently selected from nitrogen, oxygen, and sulfur, which is substituted with m instances of R′. In some embodiments, R¹ is an 8-10 membered partially aromatic or heteroaromatic bicyclic heterocyclic ring having 1-4 heteroatoms independently selected from nitrogen, oxygen, and sulfur, which is substituted with m instances of R′. In some embodiments, R¹ is —C(O)N(R″) 2. In some embodiments, R¹ is —C(O)OR″.

In some embodiments, R¹ is

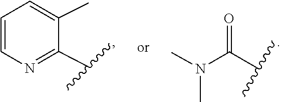

In some embodiments, R¹ is selected from those depicted in Table 1, below.

As defined generally above, R² is one of the following:
(a) C₁₋₇ aliphatic; phenyl; a 5-6 membered monocyclic heteroaryl ring having 1-4 heteroatoms independently selected from nitrogen, oxygen, and sulfur; a 3-7 membered saturated or partially unsaturated monocyclic carbocyclic ring; a 4-8 membered saturated or partially unsaturated monocyclic heterocyclic ring having 1-2 heteroatoms independently selected from nitrogen, oxygen, and sulfur; a 5-10 membered saturated or partially unsaturated bridged bicyclic ring having 0-3 heteroatoms independently selected from nitrogen, oxygen, and sulfur; a 5-10 membered saturated or partially unsaturated spirocyclic ring having 0-3 heteroatoms independently selected from nitrogen, oxygen, and sulfur; or an 8-10 membered partially aromatic or heteroaromatic bicyclic heterocyclic ring having 1-4 heteroatoms independently selected from nitrogen, oxygen, and sulfur; each of which is substituted with m instances of R′; or
(b) —C(O)N(R″) 2, or —C(O) OR″.

In some embodiments, R¹ is C₁₋₇ aliphatic, which is substituted with m instances of R′. In some embodiments, R² is phenyl, which is substituted with m instances of R′. In some embodiments, R² is a 5-6 membered monocyclic heteroaryl ring having 1-4 heteroatoms independently selected from nitrogen, oxygen, and sulfur, which is substituted with m instances of R′. In some embodiments, R² is a 3-7 membered saturated or partially unsaturated monocyclic carbocyclic ring, which is substituted with m instances of R′. In some embodiments, R² is a 4-8 membered saturated or partially unsaturated monocyclic heterocyclic ring having 1-2 heteroatoms independently selected from nitrogen, oxygen, and sulfur, which is substituted with m instances of R′. In some embodiments, R² is a 5-10 membered saturated or partially unsaturated bridged bicyclic ring having 0-3 heteroatoms independently selected from nitrogen, oxygen, and sulfur, which is substituted with m instances of R′. In some embodiments, R² is a 5-10 membered saturated or partially unsaturated spirocyclic ring having 0-3 heteroatoms independently selected from nitrogen, oxygen, and sulfur, which is substituted with m instances of R′. In some embodiments, R² is an 8-10 membered partially aromatic or heteroaromatic bicyclic heterocyclic ring having 1-4 heteroatoms independently selected from nitrogen, oxygen, and sulfur, which is substituted with m instances of R′. In some embodiments, R² is —C(O)N(R″) 2. In some embodiments, R² is —C(O) OR″.

In some embodiments, R² is

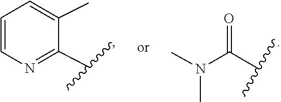

In some embodiments, R² is selected from those depicted in Table 1, below.

As defined generally above, R′ is hydrogen, halogen, or a C₁₋₆ aliphatic group optionally substituted with 1-5 halogens.

In some embodiments, R′ is hydrogen, halogen, or a C₁₋₆ aliphatic group optionally substituted with 1-5 halogens. In some embodiments, R′ is hydrogen. In some embodiments, R′ is halogen. In some embodiments, R′ is C₁₋₆ aliphatic group optionally substituted with 1-5 halogens.

In some embodiments, R′ is selected from those depicted in Table 1, below.

As defined generally above, R″ is hydrogen, or a C₁₋₆ aliphatic group optionally substituted with 1-5 halogens.

In some embodiments, R″ is hydrogen, or a C₁₋₆ aliphatic group optionally substituted with 1-5 halogens. In some embodiments, R″ is hydrogen. In some embodiments, R″ is C₁₋₆ aliphatic group optionally substituted with 1-5 halogens.

In some embodiments, R″ is selected from those depicted in Table 1, below.

As defined generally above, each R³ is independently —N(R)₂, —OR, or C₁₋₇ aliphatic optionally substituted with 1-5 groups independently selected from halogen, —N(R)₂, or —OR, and wherein 1 or 2 methylene units of the aliphatic group are optionally replaced by —N(R)— or a methyl group is optionally replaced by —N(R)₂.

In some embodiments, each R³ is independently —N(R)₂. In some embodiments, each R³ is independently —OR. In some embodiments, each R³ is independently C₁₋₇ aliphatic optionally substituted with 1-5 groups independently selected from halogen, —N(R)₂, or —OR, and wherein 1 or 2 methylene units of the aliphatic group are optionally replaced by —N(R)— or a methyl group is optionally replaced by —N(R)₂.

In some embodiments, R³ is

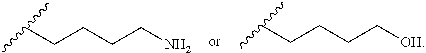 [SMILES: *CCCCN ; *CCCCO]

In some embodiments, R³ is selected from those depicted in Table 1, below.

As defined generally above, in some embodiments, [symbol] is selected from the following:

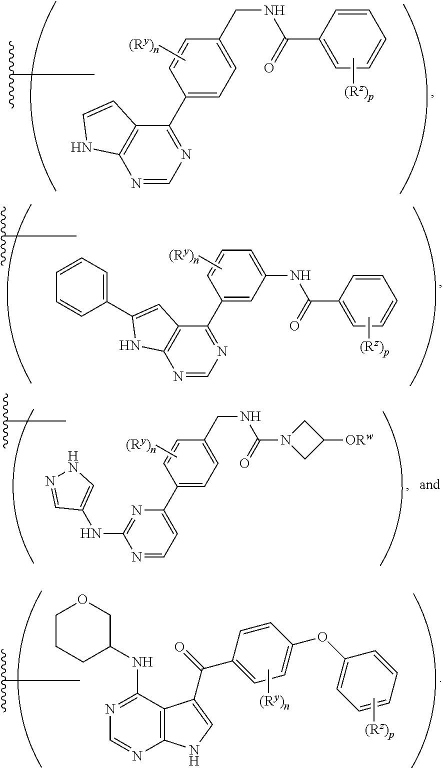

In some embodiments, [symbol] is

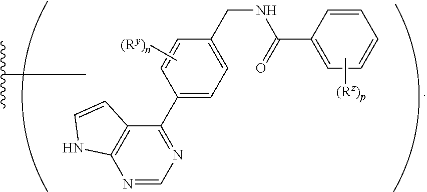

In some embodiments, [symbol] is

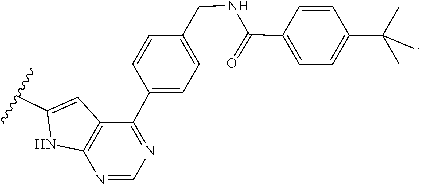 [SMILES: *c1cc2c(-c3ccc(CNC(=O)c4ccc(C(C)(C)C)cc4)cc3)ncnc2[nH]1]

In some embodiments, [symbol] is

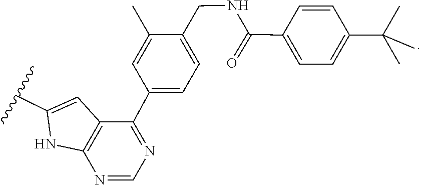 [SMILES: *c1cc2c(-c3ccc(CNC(=O)c4ccc(C(C)(C)C)cc4)c(C)c3)ncnc2[nH]1]

In some embodiments, [symbol] is

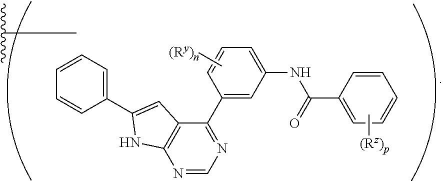

In some embodiments, [symbol] is

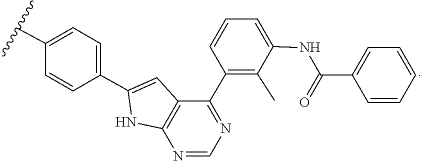 [SMILES: *c1ccc(-c2cc3c(-c4cccc(NC(=O)c5ccccc5)c4C)ncnc3[nH]2)cc1]

In some embodiments, [symbol] is

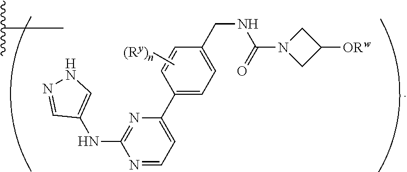

In some embodiments, [symbol] is

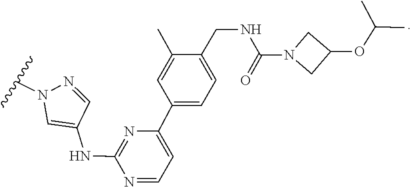 [SMILES: *n1cc(Nc2nccc(-c3ccc(CNC(=O)N4CC(OC(C)C)C4)c(C)c3)n2)cn1]

In some embodiments, [symbol] is

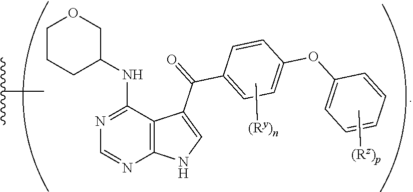

In some embodiments, [symbol] is

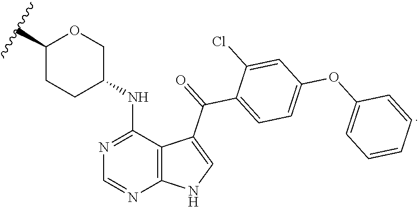 [SMILES: *[C@@H]1CC[C@@H](Nc2ncnc3[nH]cc(C(=O)c4ccc(Oc5ccccc5)cc4Cl)c23)CO1]

In some embodiments, [symbol] is selected from those depicted in Table 1, below.

As defined generally above, R^{w} is hydrogen, or C₁₋₇ aliphatic optionally substituted with 1 to 5 halogens.

In some embodiments, R is hydrogen, or C₁₋₇ aliphatic optionally substituted with 1 to 5 halogens. In some embodiments, R″ is hydrogen. In some embodiments, R^{w} is C₁₋₇ aliphatic optionally substituted with 1 to 5 halogens.

In some embodiments, R^{w} is selected from those depicted in Table 1, below.

As defined generally above, R^{y} is hydrogen, halogen, or C₁₋₇ aliphatic optionally substituted with 1 to 5 halogens.

In some embodiments, R^{y} is hydrogen, halogen, or C₁₋₇ aliphatic optionally substituted with 1 to 5 halogens. In some embodiments, R^{y} is hydrogen. In some embodiments, R^{y} is halogen. In some embodiments, R^{y} is C₁₋₇ aliphatic optionally substituted with 1 to 5 halogens.

In some embodiments, R^{y} is selected from those depicted in Table 1, below.

As defined generally above, R^{z} is hydrogen, halogen, or C₁₋₇ aliphatic optionally substituted with 1 to 5 halogens.

In some embodiments, R^{z} is hydrogen, halogen, or C₁₋₇ aliphatic optionally substituted with 1 to 5 halogens. In some embodiments, R^{z} is hydrogen. In some embodiments, R^{z} is halogen. In some embodiments, R^{z} is C₁₋₇ aliphatic optionally substituted with 1 to 5 halogens.

In some embodiments, R^{z} is selected from those depicted in Table 1, below.

As defined generally above, j is 0, 1, or 2.

In some embodiments, j is 0, 1, or 2. In some embodiments, j is 0. In some embodiments, j is 1. In some embodiments, j is 2.

In some embodiments, j is selected from those depicted in Table 1, below.

As defined generally above, m is 0, 1, 2, 3, or 4.

In some embodiments, m is 0, 1, 2, 3, or 4. In some embodiments, m is 0. In some embodiments, m is 1. In some embodiments, m is 2. In some embodiments, m is 3. In some embodiments, m is 4.

In some embodiments, m is selected from those depicted in Table 1, below.

As defined generally above, n is 0, 1, 2, 3, or 4.

In some embodiments, n is 0, 1, 2, 3, or 4. In some embodiments, n is 0. In some embodiments, n is 1. In some embodiments, n is 2. In some embodiments, n is 3. In some embodiments, n is 4.

In some embodiments, n is selected from those depicted in Table 1, below.

As defined generally above, p is 0, 1, 2, 3, 4, or 5.

In some embodiments, p is 0, 1, 2, 3, 4, or 5. In some embodiments, p is 0. In some embodiments, p is 1. In some embodiments, p is 2. In some embodiments, p is 3. In some embodiments, p is 4. In some embodiments, p is 5.

In some embodiments, p is selected from those depicted in Table 1, below.

In some embodiments, the present invention provides a compound of formula I-A, I-B, I-C, or I-D:

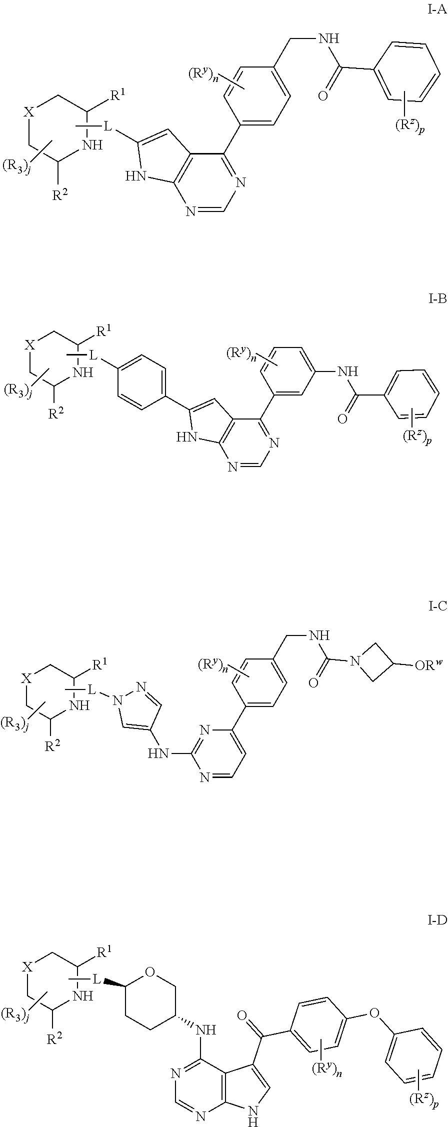

or a pharmaceutically acceptable salt, solvate, or hydrate thereof, wherein each of X, -L-, R¹, R², R³, R^{w}, R^{y}, R^{z}, j, n, and p is as defined above and described in embodiments herein, both singly and in combination.

In some embodiments, the present invention provides a compound of formula I-A-a, I-B-a, I-C-a, or I-D-a:

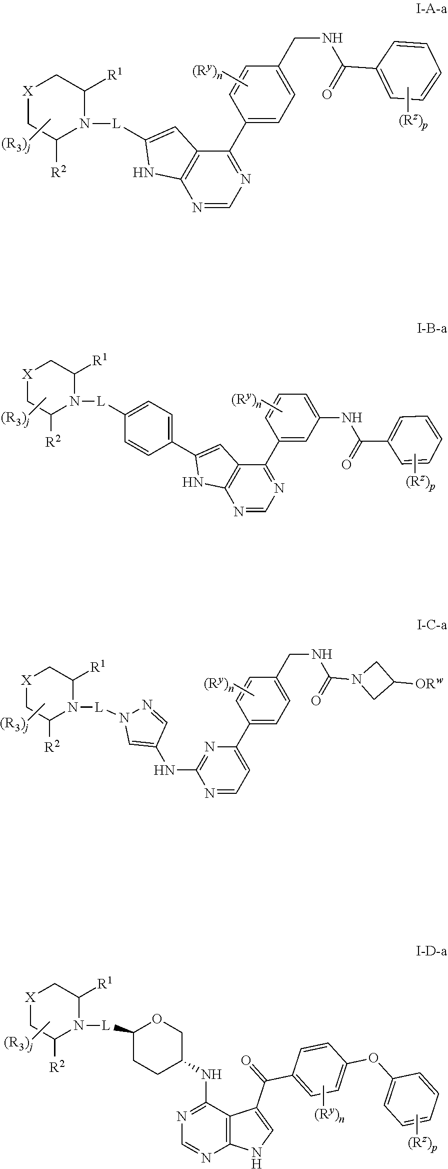

or a pharmaceutically acceptable salt, solvate, or hydrate thereof, wherein each of X, -L-, R¹, R², R³, R^{w}, R^{y}, R^{z}, j, n, and p is as defined above and described in embodiments herein, both singly and in combination.

In some embodiments, the present invention provides a compound of formula I-A-b, I-B-b, I-C-b, or I-D-b:

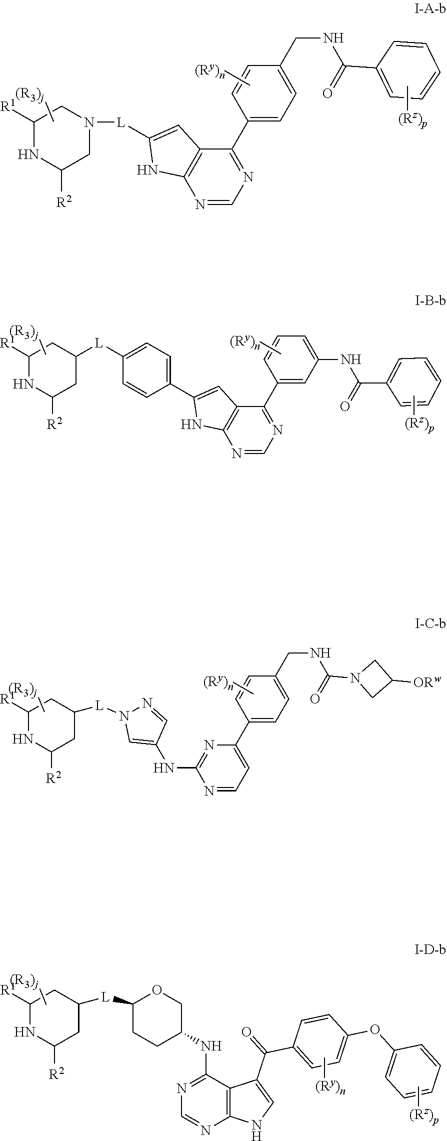

or a pharmaceutically acceptable salt, solvate, or hydrate thereof, wherein each of -L-, R¹, R², R³, R^{w}, R^{y}, R^{z}, j, n, and p is as defined above and described in embodiments herein, both singly and in combination.

In some embodiments, the present invention provides a compound of formula I-A-c, I-A-d, I-A-e, or I-A-f:

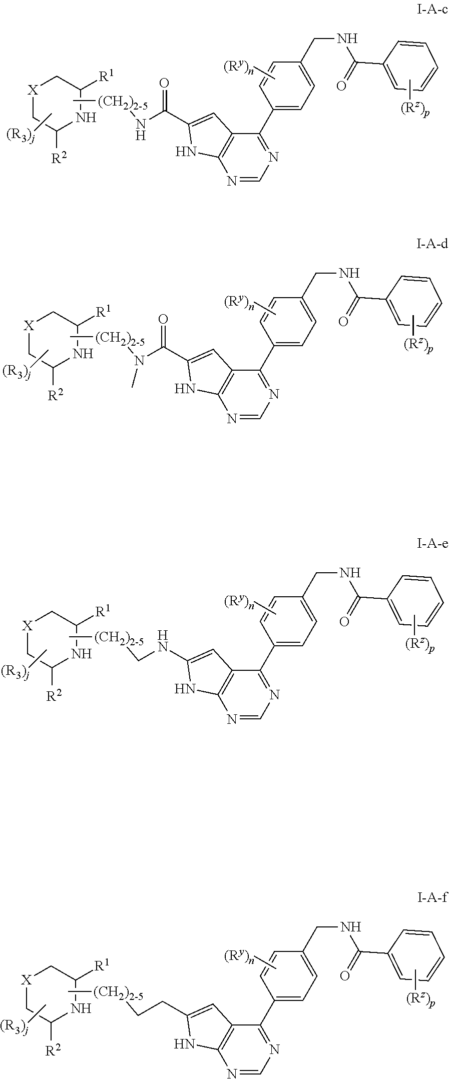

or a pharmaceutically acceptable salt, solvate, or hydrate thereof, wherein each of X, R¹, R², R³, R^{w}, R^{y}, R^{z}, j, n, and p is as defined above and described in embodiments herein, both singly and in combination.

In some embodiments, the present invention provides a compound of formula I-A-g, I-A-h, I-A-i, or I-A-j:

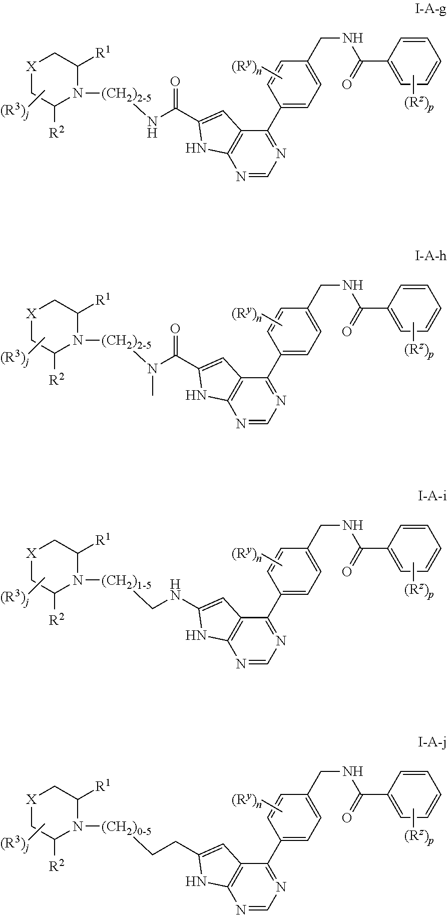

or a pharmaceutically acceptable salt, solvate, or hydrate thereof, wherein each of X, R¹, R², R³, R^{w}, R^{y}, R^{z}, j, n, and p is as defined above and described in embodiments herein, both singly and in combination.

In some embodiments, the present invention provides a compound of formula I-A-k, I-A-l, I-A-m, or I-A-n:

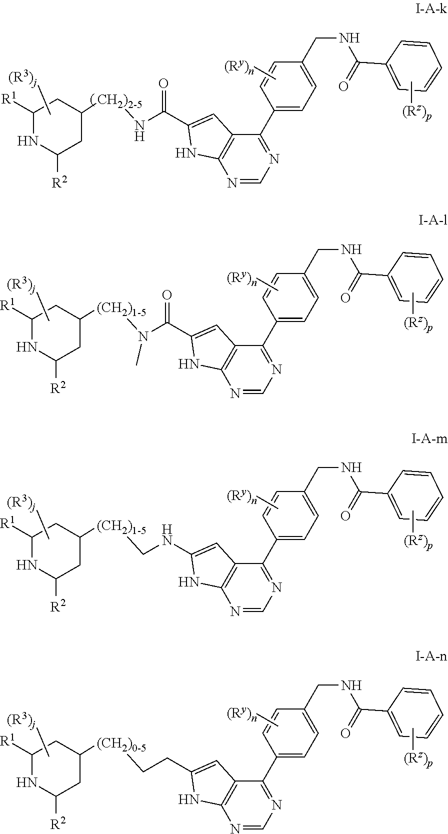

or a pharmaceutically acceptable salt, solvate, or hydrate thereof, wherein each of R¹, R², R³, R^{w}, R^{y}, R^{z}, j, n, and p is as defined above and described in embodiments herein, both singly and in combination.

In some embodiments, the present invention provides a compound of formula I-A-o, I-A-p, I-A-q, or I-A-r:

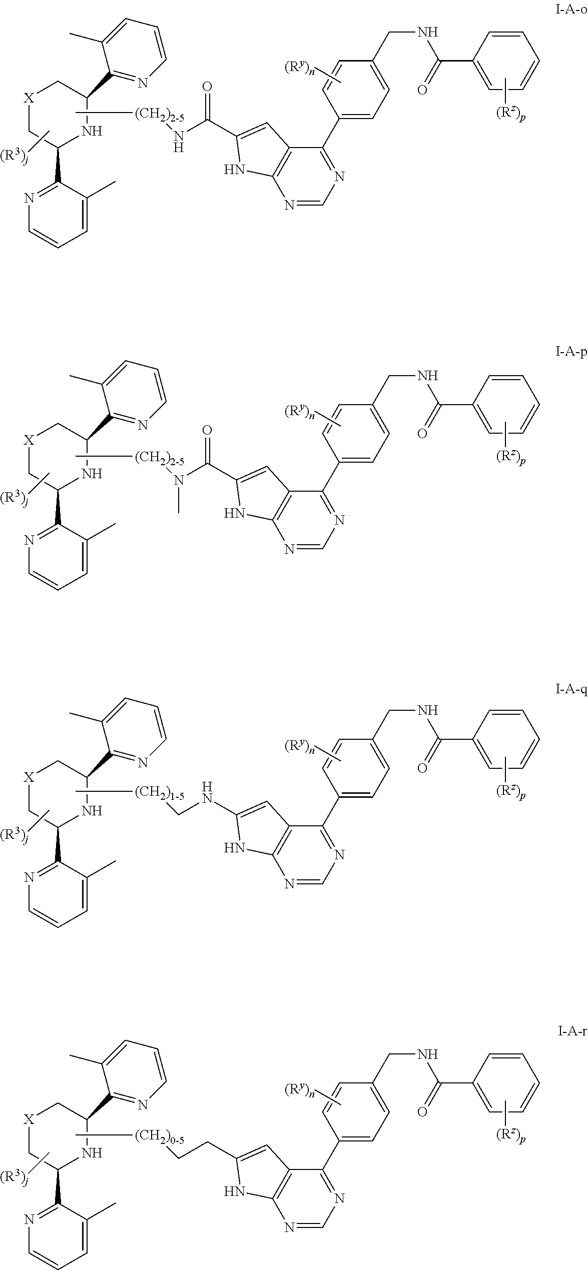

or a pharmaceutically acceptable salt, solvate, or hydrate thereof, wherein each of X, R³, R^{w}, R^{y}, R^{z}, j, n, and p is as defined above and described in embodiments herein, both singly and in combination.

In some embodiments, the present invention provides a compound of formula I-A-s, I-A-t, I-A-u, or I-A-v:

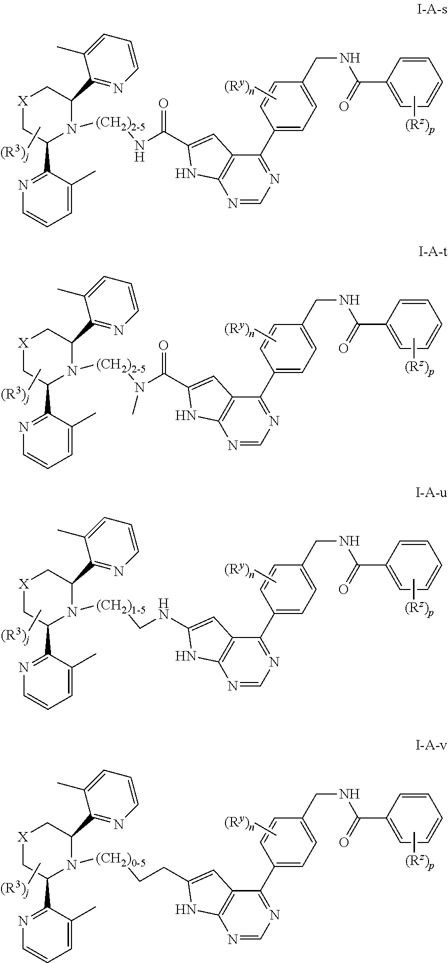

or a pharmaceutically acceptable salt, solvate, or hydrate thereof, wherein each of X, R³, R^{w}, R^{y}, R^{z}, j, n, and p is as defined above and described in embodiments herein, both singly and in combination.

In some embodiments, the present invention provides a compound of formula I-A-w, I-A-x, I-A-y, or I-A-z:

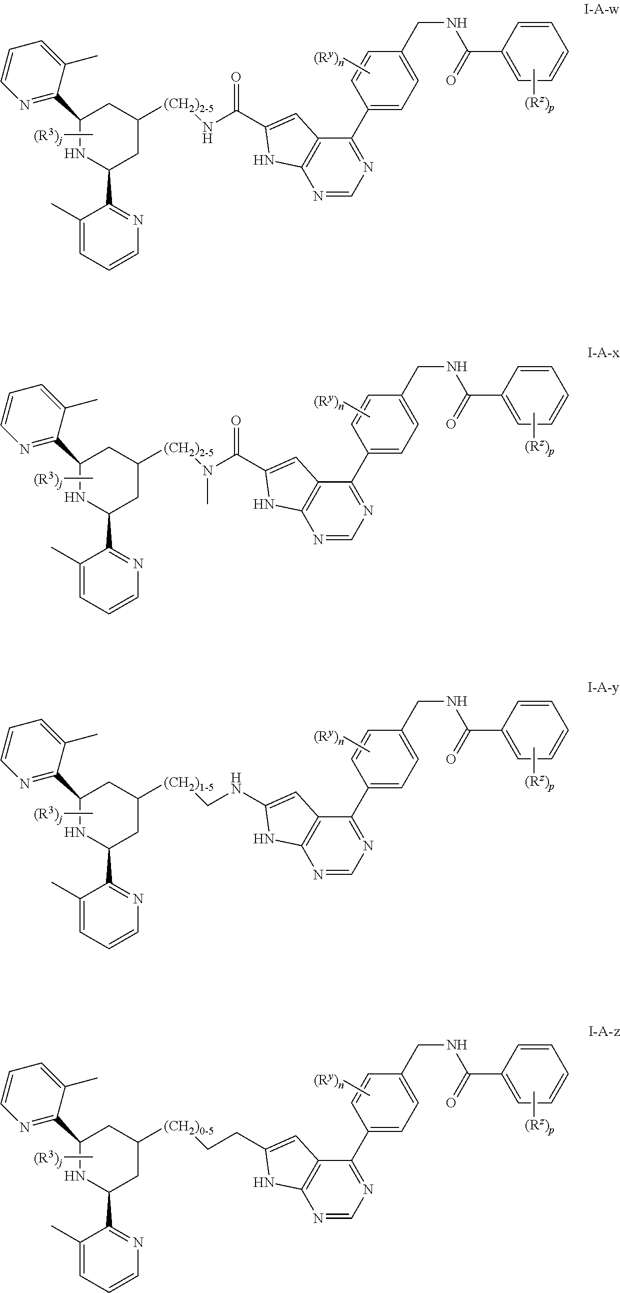

or a pharmaceutically acceptable salt, solvate, or hydrate thereof, wherein each of R³, R^{w}, R^{y}, R^{z}, j, n, and p is as defined above and described in embodiments herein, both singly and in combination.

In some embodiments, the present invention provides a compound of formula I-B-c, I-B-d, I-B-e, or I-B-f:

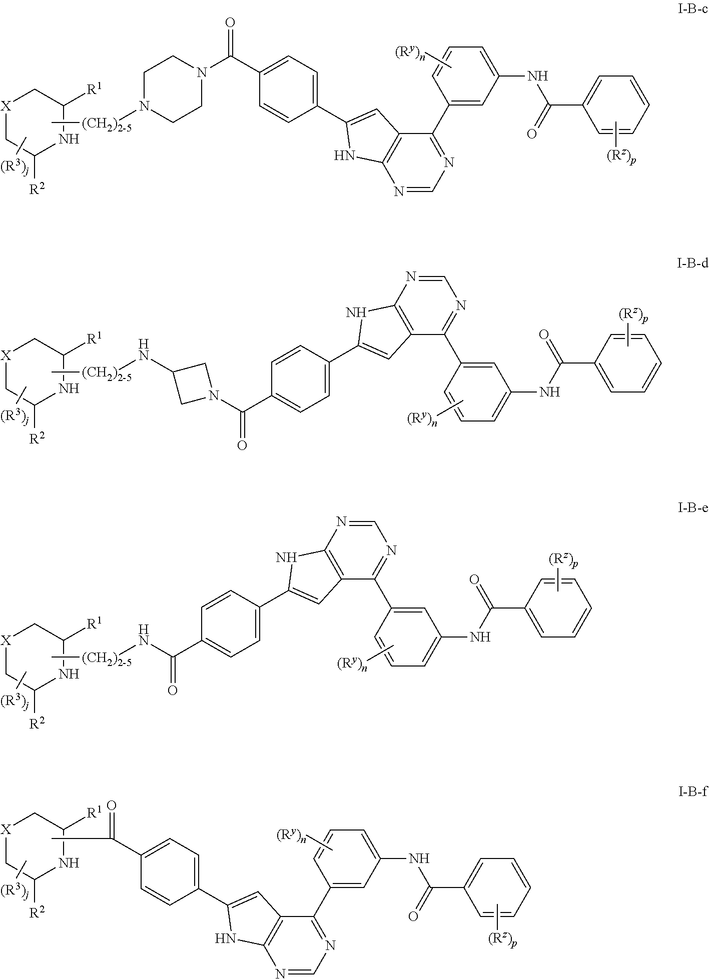

or a pharmaceutically acceptable salt, solvate, or hydrate thereof, wherein each of X, R¹, R², R³, R^{y}, R^{z}, j, n, and p is as defined above and described in embodiments herein, both singly and in combination.

In some embodiments, the present invention provides a compound of formula I-B-g, I-B-h, I-B-i, or I-B-j:

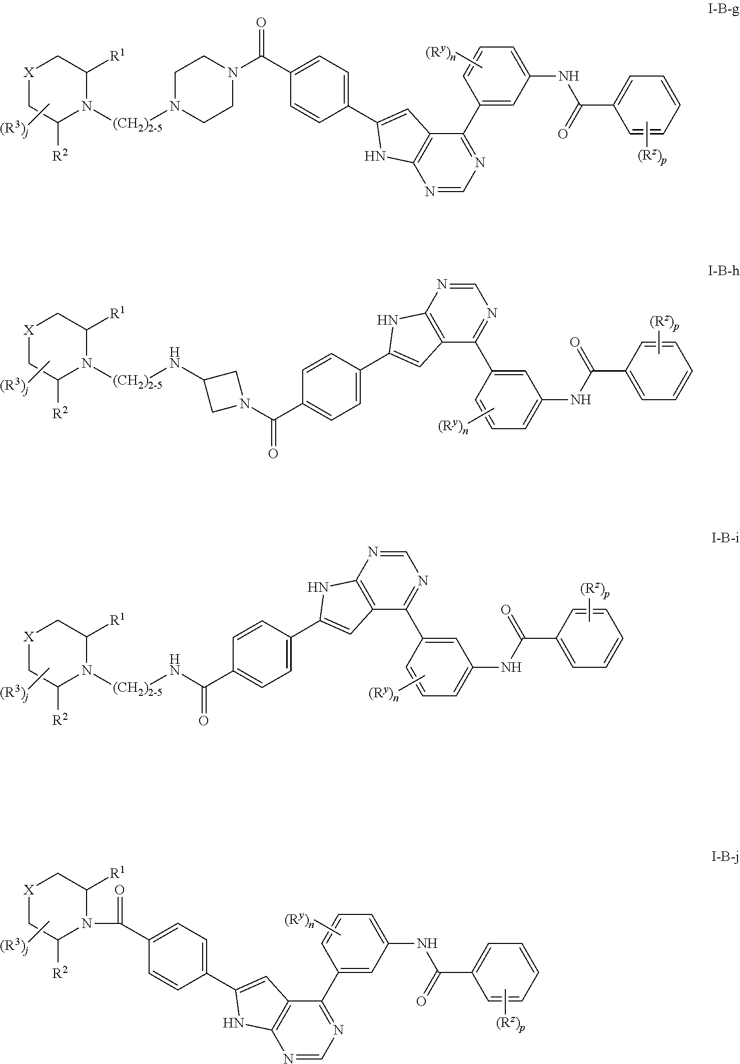

or a pharmaceutically acceptable salt, solvate, or hydrate thereof, wherein each of X, R¹, R², R³, R^{y}, R^{z}, j, n, and p is as defined above and described in embodiments herein, both singly and in combination.

In some embodiments, the present invention provides a compound of formula I-B-k, I-B-l, I-B-m, or I-B-n:

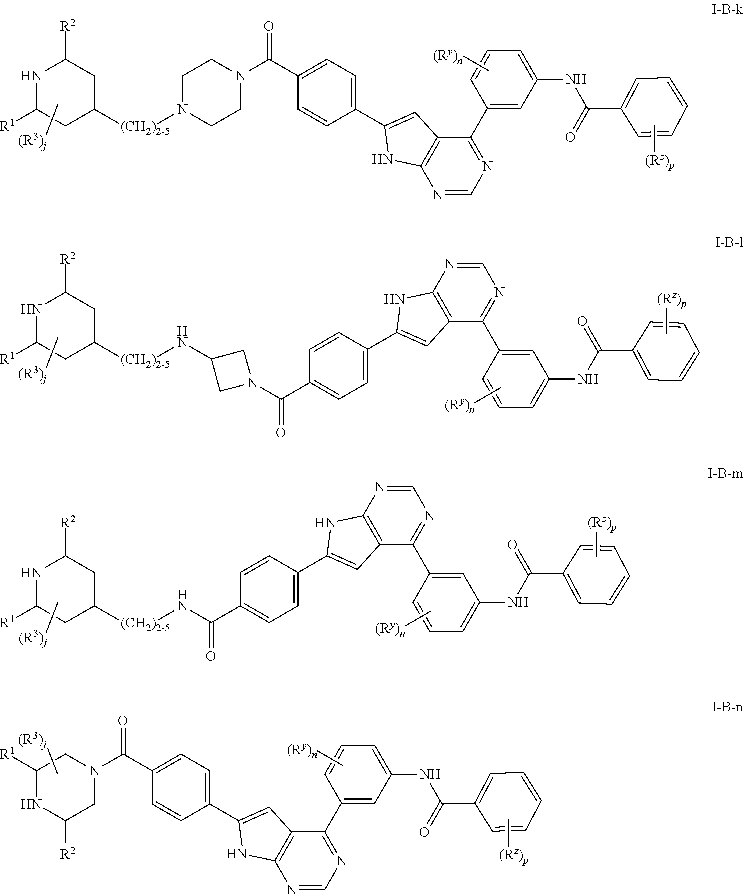

or a pharmaceutically acceptable salt, solvate, or hydrate thereof, wherein each of R¹, R², R³, R^{y}, R², j, n, and p is as defined above and described in embodiments herein, both singly and in combination.

In some embodiments, the present invention provides a compound of formula I-B-o, I-B-p, I-B-q, or I-B-r:

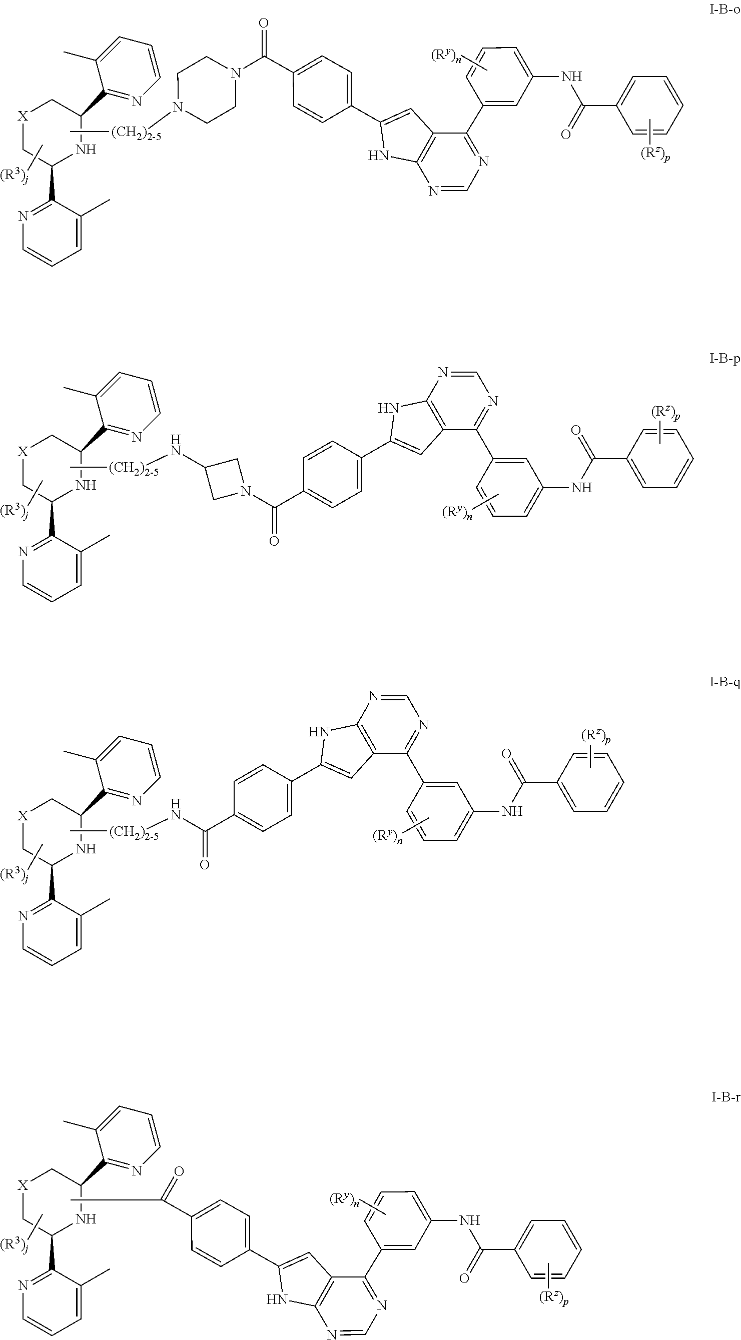

or a pharmaceutically acceptable salt, solvate, or hydrate thereof, wherein each of X, R³, R^{y}, R^{z}, j, n, and p is as defined above and described in embodiments herein, both singly and in combination.

In some embodiments, the present invention provides a compound of formula I-B-s, I-B-t, I-B-u, or I-B-v:

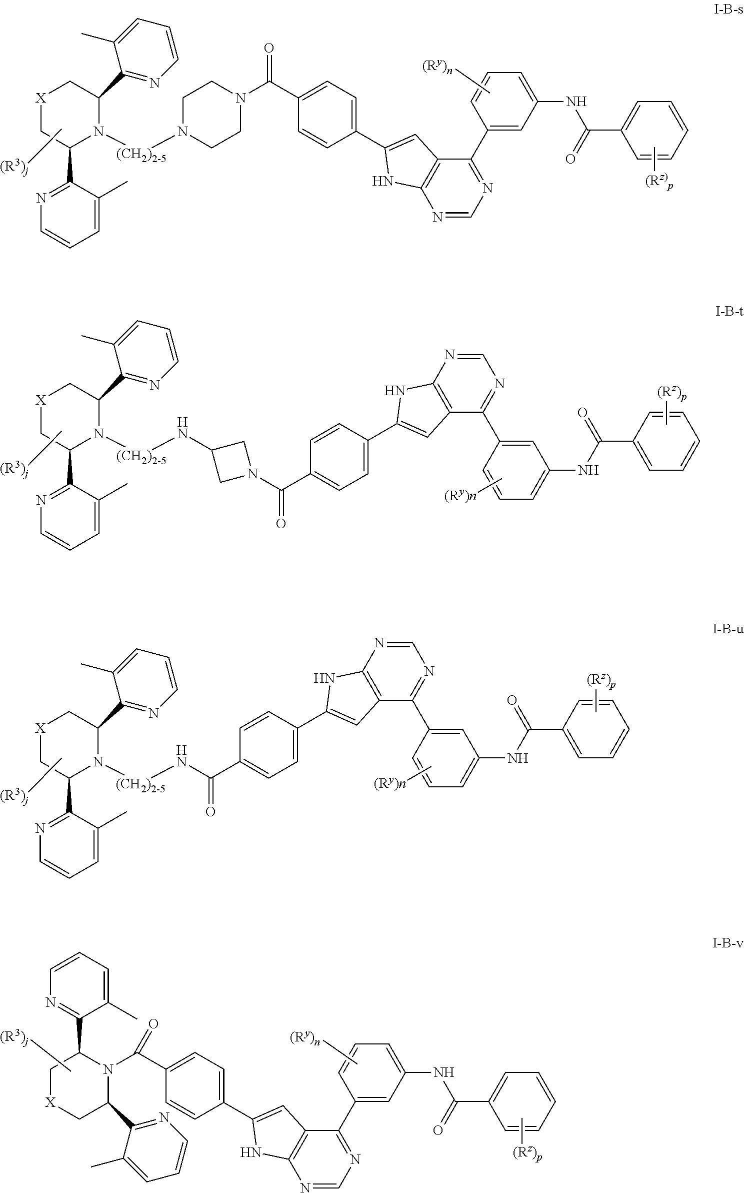

or a pharmaceutically acceptable salt, solvate, or hydrate thereof, wherein each of X, R³, R^{y}, R^{z}, j, n, and p is as defined above and described in embodiments herein, both singly and in combination.

In some embodiments, the present invention provides a compound of formula I-B-w, I-B-x, I-B-y, or I-B-z:

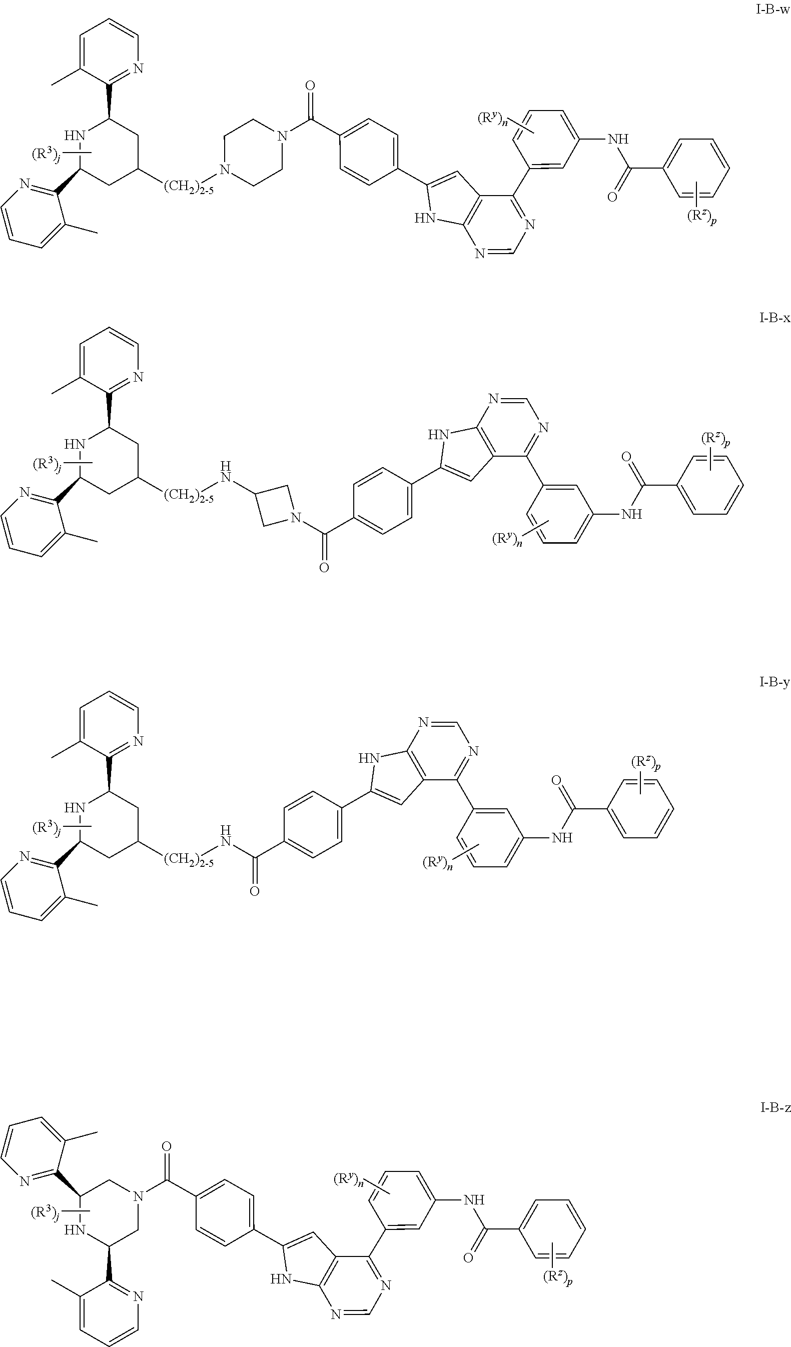

or a pharmaceutically acceptable salt, solvate, or hydrate thereof, wherein each of R³, R^{y}, R^{z}, j, n, and p is as defined above and described in embodiments herein, both singly and in combination.

In some embodiments, the present invention provides a compound of formula I-C-c:

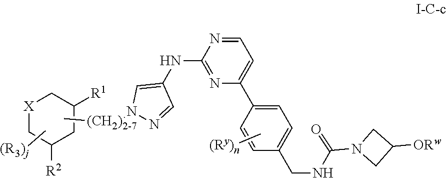

or a pharmaceutically acceptable salt, solvate, or hydrate thereof, wherein each of X, R¹, R², R³, R^{y}, R^{z}, j, n, and p is as defined above and described in embodiments herein, both singly and in combination.

In some embodiments, the present invention provides a compound of formula I-C-d:

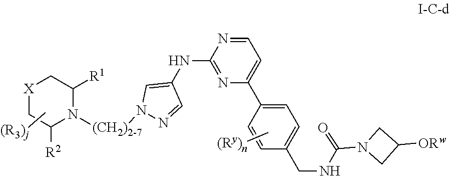

or a pharmaceutically acceptable salt, solvate, or hydrate thereof, wherein each of X, R¹, R², R³, R^{y}, R^{z}, j, n, and p is as defined above and described in embodiments herein, both singly and in combination.

In some embodiments, the present invention provides a compound of formula I-C-e:

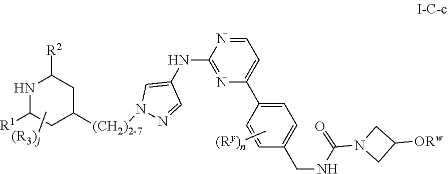

or a pharmaceutically acceptable salt, solvate, or hydrate thereof, wherein each of X, R¹, R², R³, R^{y}, R^{z}, j, n, and p is as defined above and described in embodiments herein, both singly and in combination.

In some embodiments, the present invention provides a compound of formula I-C-f:

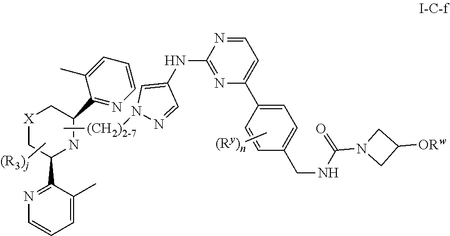

or a pharmaceutically acceptable salt, solvate, or hydrate thereof, wherein each of X, R³, R^{y}, R^{z}, j, n, and p is as defined above and described in embodiments herein, both singly and in combination. In some embodiments, the present invention provides a compound of formula I-C-g:

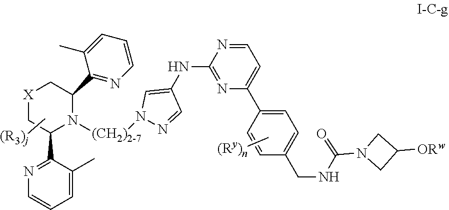

or a pharmaceutically acceptable salt, solvate, or hydrate thereof, wherein each of X, R³, R^{y}, R^{z}, j, n, and p is as defined above and described in embodiments herein, both singly and in combination.

In some embodiments, the present invention provides a compound of formula I-C-h:

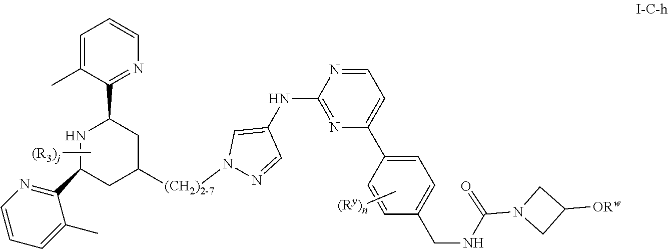

or a pharmaceutically acceptable salt, solvate, or hydrate thereof, wherein each of R³, R^{y}, R^{z}, j, n, and p is as defined above and described in embodiments herein, both singly and in combination.

In some embodiments, the present invention provides a compound of formula I-D-c, I-D-d, I-D-e, I-D-f, or I-B-g:

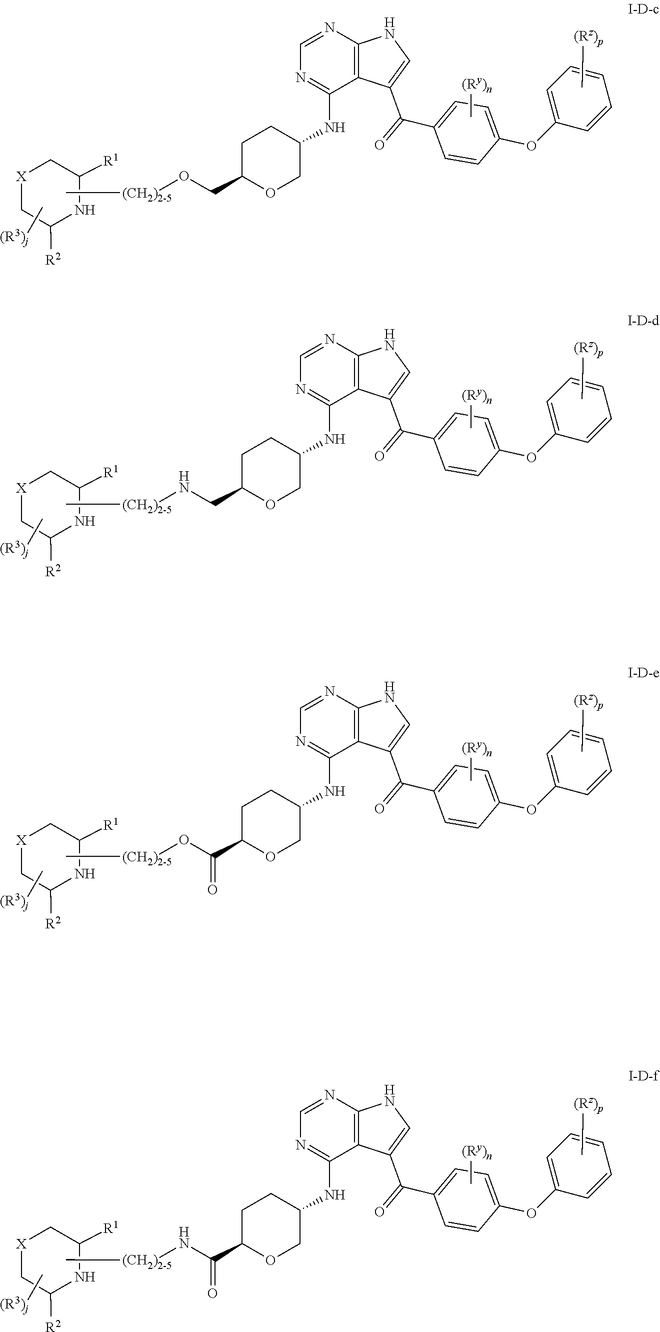

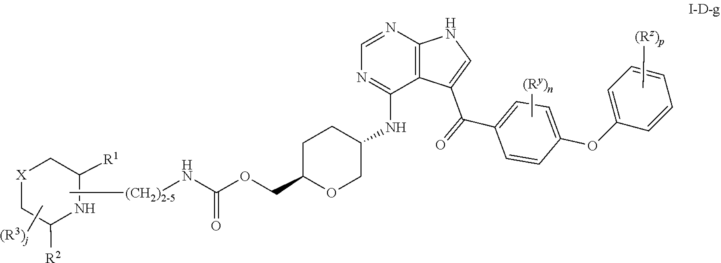

or a pharmaceutically acceptable salt, solvate, or hydrate thereof, wherein each of X, R¹, R², R³, R^{y}, R^{z}, j, n, and p is as defined above and described in embodiments herein, both singly and in combination.

In some embodiments, the present invention provides a compound of formula I-D-h, I-D-i, I-D-j, I-D-k, or I-B-l:

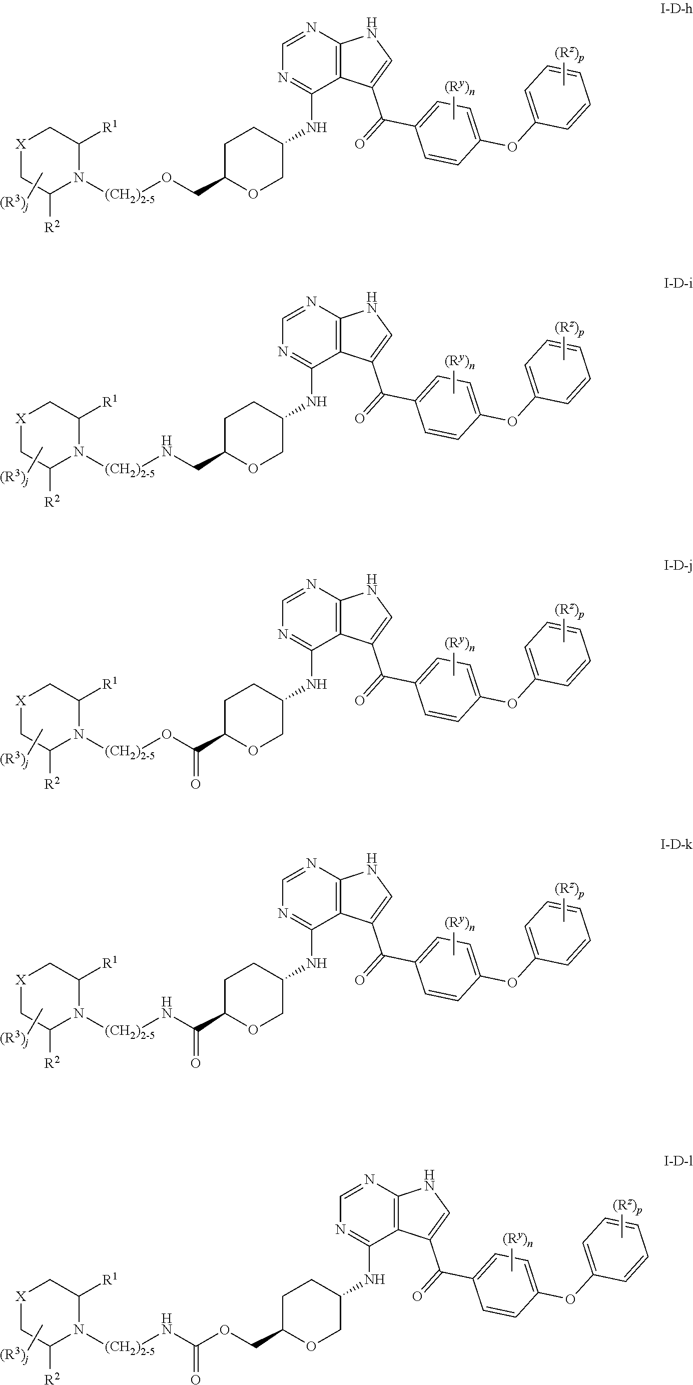

or a pharmaceutically acceptable salt, solvate, or hydrate thereof, wherein each of X, R¹, R², R³, R^{y}, R^{z}, j, n, and p is as defined above and described in embodiments herein, both singly and in combination.

In some embodiments, the present invention provides a compound of formula I-D-m, I-D-n, I-D-o, I-D-p, or I-D-q:

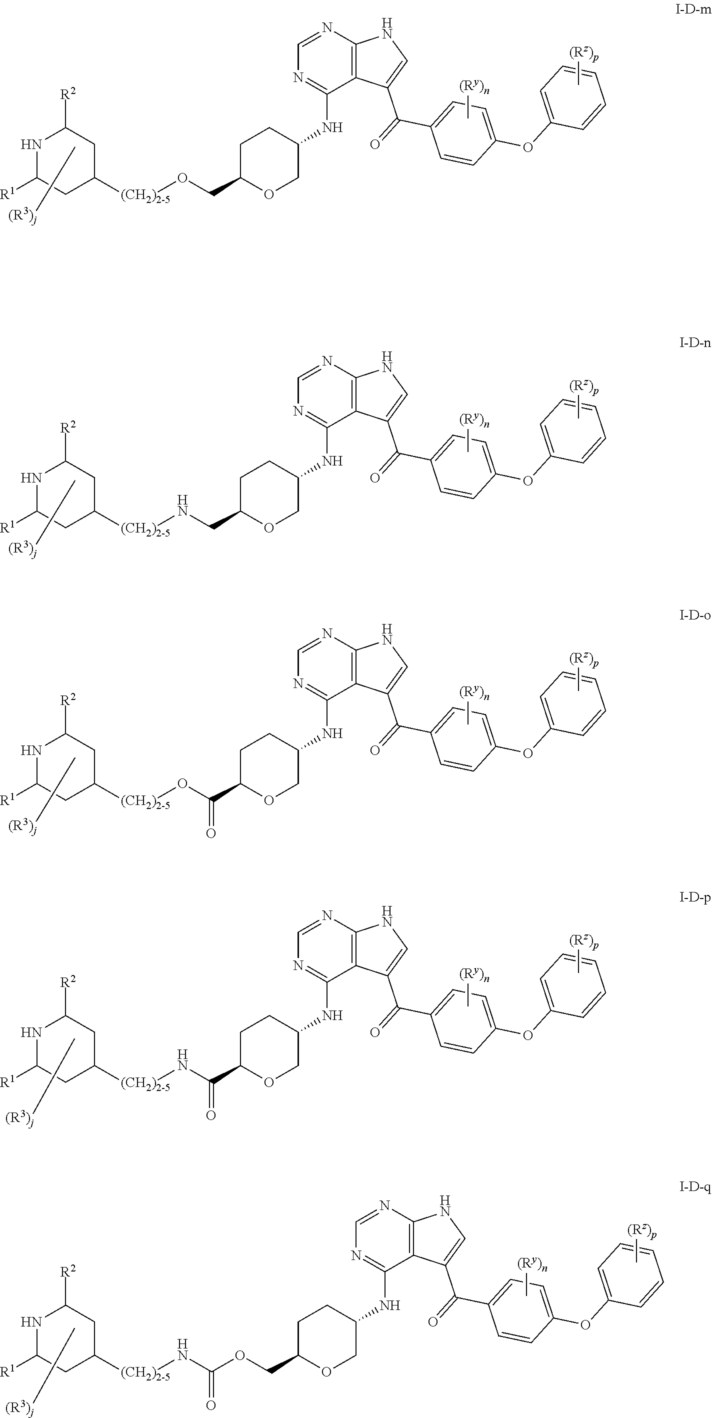

or a pharmaceutically acceptable salt, solvate, or hydrate thereof, wherein each of R¹, R², R³, R^{y}, R^{z}, j, n, and p is as defined above and described in embodiments herein, both singly and in combination.

In some embodiments, the present invention provides a compound of formula I-D-r, I-D-s, I-D-t, I-D-u, or I-D-v:

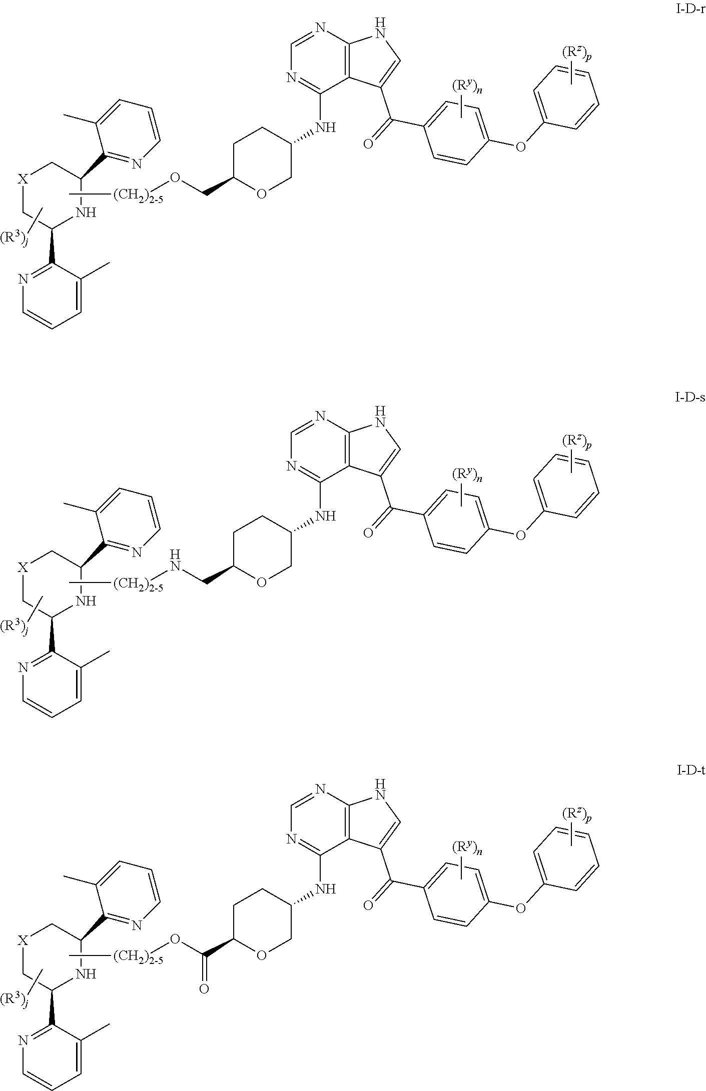

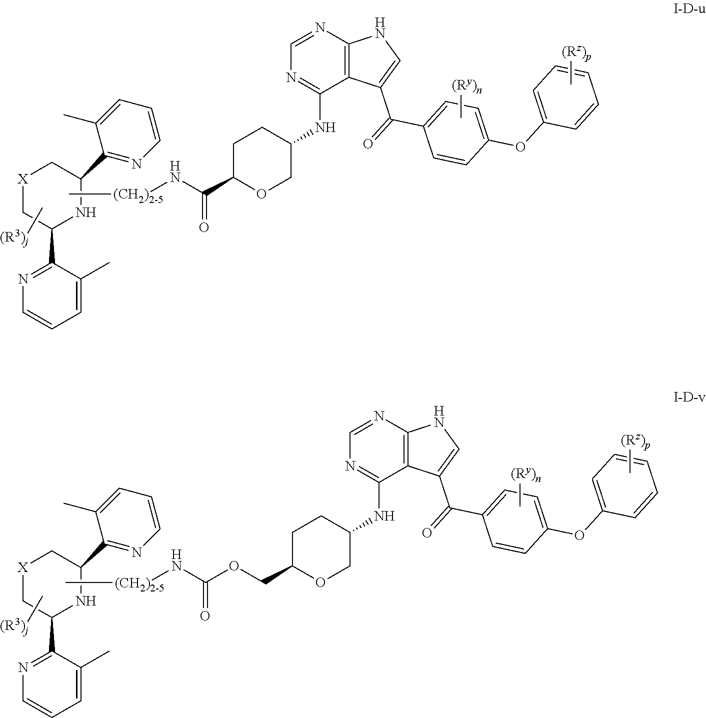

or a pharmaceutically acceptable salt, solvate, or hydrate thereof, wherein each of X, R³, R^{y}, R^{z}, j, n, and p is as defined above and described in embodiments herein, both singly and in combination.

In some embodiments, the present invention provides a compound of formula I-D-r-a, I-D-s-a, I-D-t-a, I-D-u-a, or I-D-v-a:

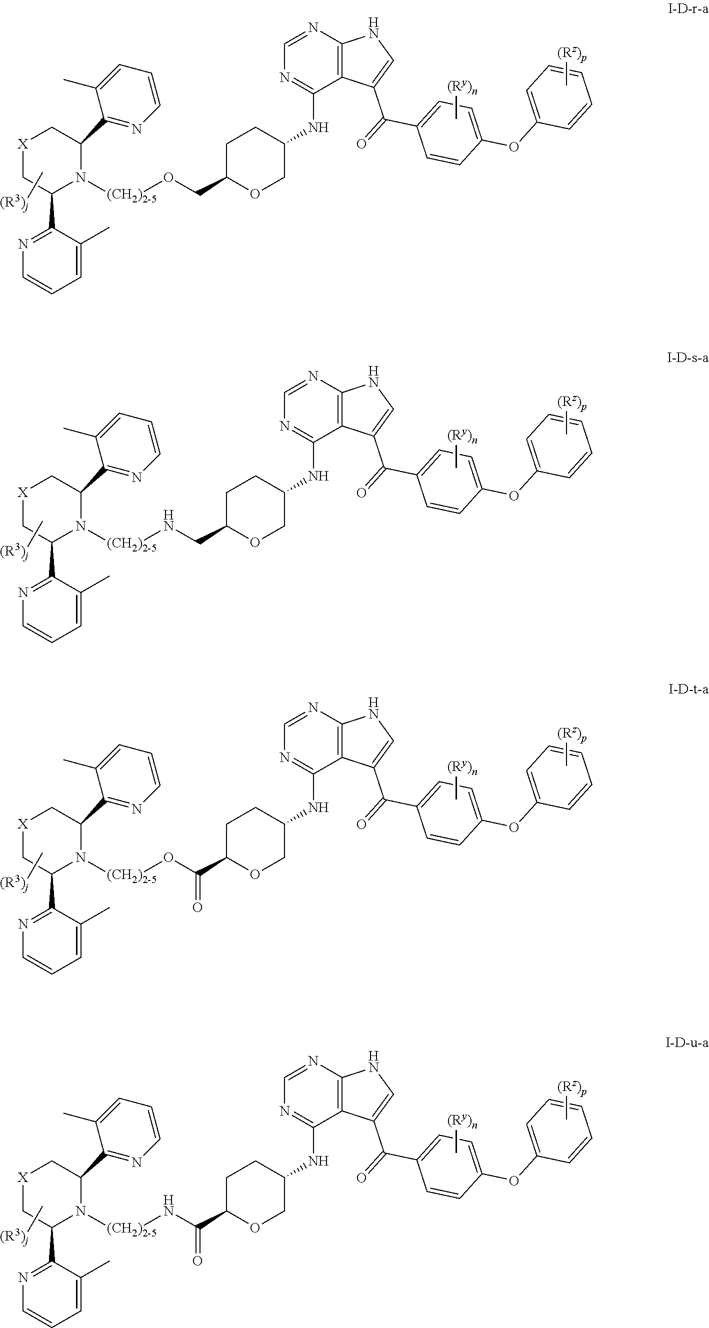

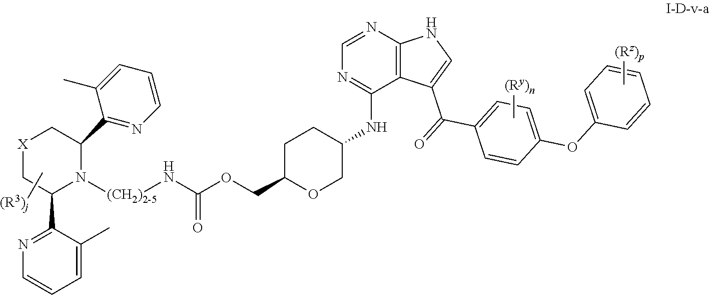

or a pharmaceutically acceptable salt, solvate, or hydrate thereof, wherein each of X, R³, R^{y}, R^{z}, j, n, and p is as defined above and described in embodiments herein, both singly and in combination.

In some embodiments, the present invention provides a compound of formula I-D-r-b, I-D-s-b, I-D-t-b, I-D-u-b, or I-D-v-b:

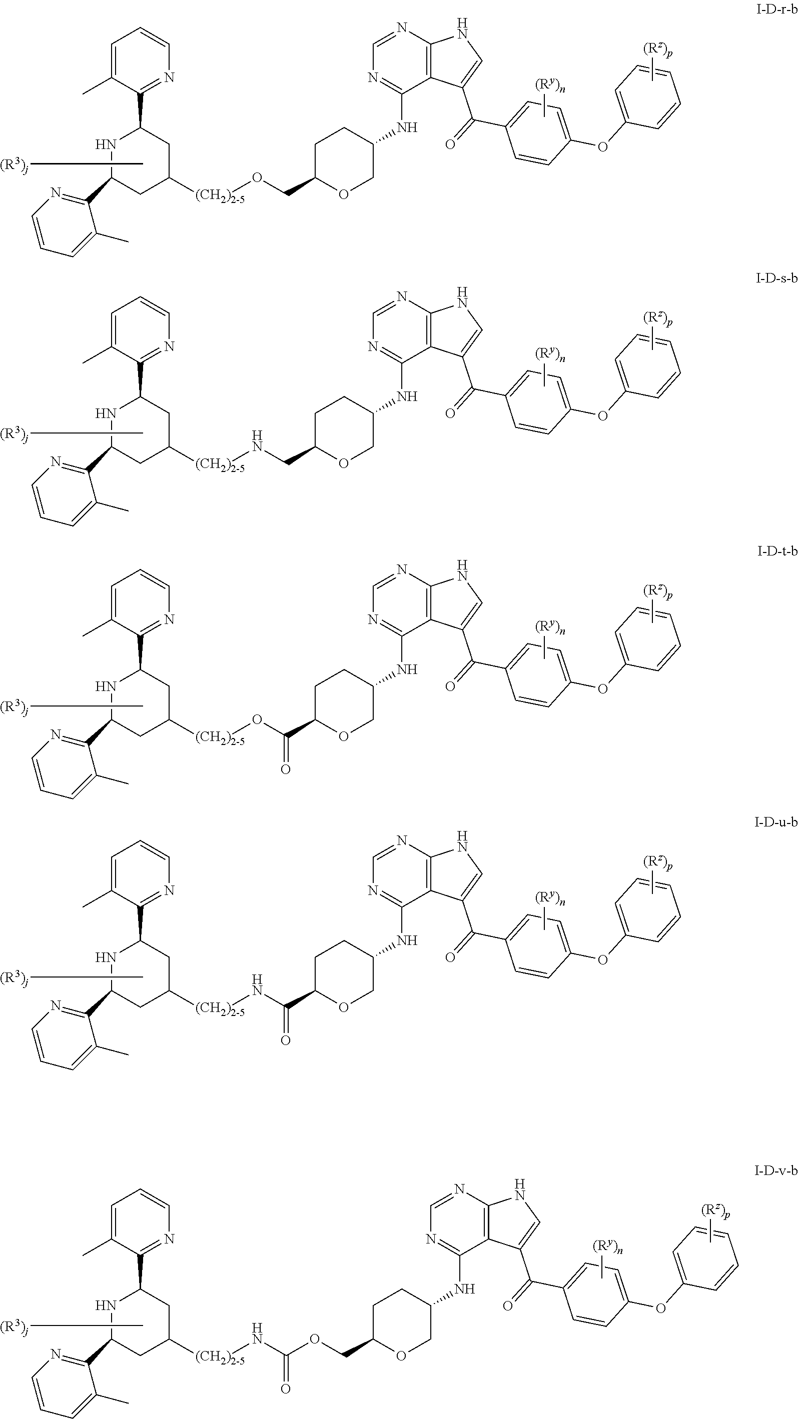

or a pharmaceutically acceptable salt, solvate, or hydrate thereof, wherein each of R³, R^{y}, R^{z}, j, n, and p is as defined above and described in embodiments herein, both singly and in combination. Exemplary compounds of the invention are set forth in Table 1, below.

TABLE 1
Exemplary Compounds

| Compound | |
|---|---|
| No. | Structure |
| I-1 | 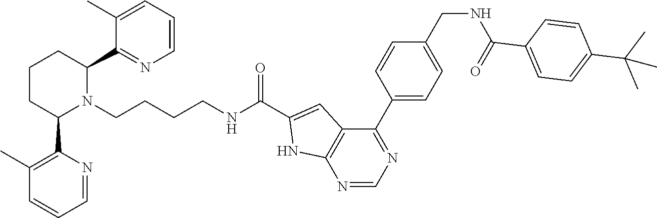 [SMILES: Cc1cccnc1[C@H]1CCC[C@@H](c2ncccc2C)N1CCCCNC(=O)c1cc2c(-c3ccc(CNC(=O)c4ccc(C(C)(C)C)cc4)cc3)ncnc2[nH]1] |
| I-2 | 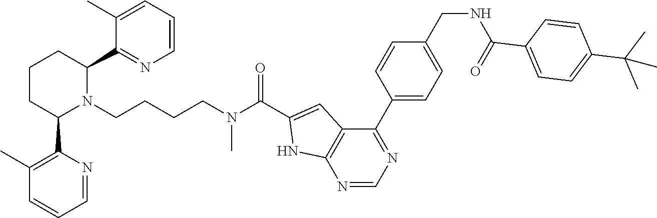 [SMILES: Cc1cccnc1[C@H]1CCC[C@@H](c2ncccc2C)N1CCCCN(C)C(=O)c1cc2c(-c3ccc(CNC(=O)c4ccc(C(C)(C)C)cc4)cc3)ncnc2[nH]1] |
| I-3 | 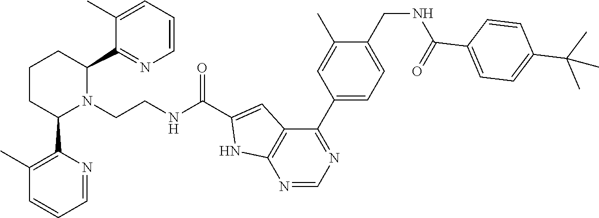 [SMILES: Cc1cc(-c2ncnc3[nH]c(C(=O)NCCN4[C@@H](c5ncccc5C)CCC[C@H]4c4ncccc4C)cc23)ccc1CNC(=O)c1ccc(C(C)(C)C)cc1] |
| I-4 | 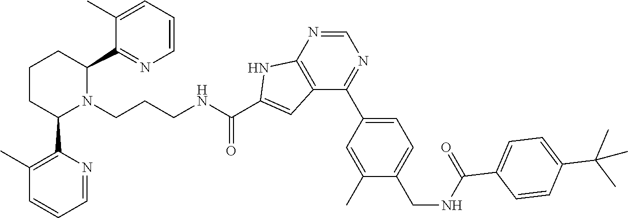 [SMILES: Cc1cc(-c2ncnc3[nH]c(C(=O)NCCCN4[C@@H](c5ncccc5C)CCC[C@H]4c4ncccc4C)cc23)ccc1CNC(=O)c1ccc(C(C)(C)C)cc1] |
| I-5 | 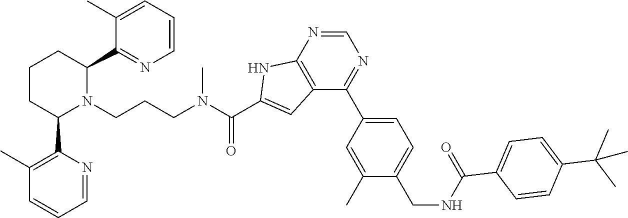 [SMILES: Cc1cc(-c2ncnc3[nH]c(C(=O)N(C)CCCN4[C@@H](c5ncccc5C)CCC[C@H]4c4ncccc4C)cc23)ccc1CNC(=O)c1ccc(C(C)(C)C)cc1] |
| I-6 | 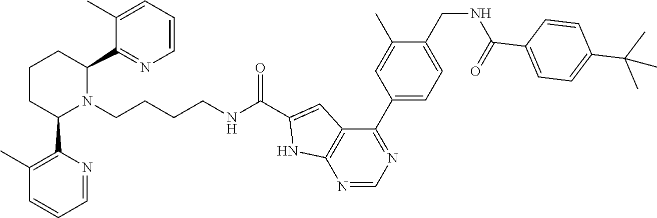 [SMILES: Cc1cc(-c2ncnc3[nH]c(C(=O)NCCCCN4[C@@H](c5ncccc5C)CCC[C@H]4c4ncccc4C)cc23)ccc1CNC(=O)c1ccc(C(C)(C)C)cc1] |
| I-7 | 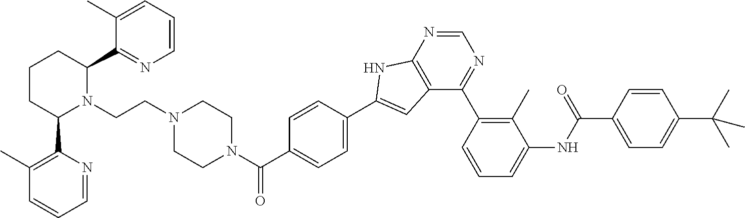 [SMILES: Cc1cccnc1[C@H]1CCC[C@@H](c2ncccc2C)N1CCN1CCN(C(=O)c2ccc(-c3cc4c(-c5cccc(NC(=O)c6ccc(C(C)(C)C)cc6)c5C)ncnc4[nH]3)cc2)CC1] |
| I-8 | 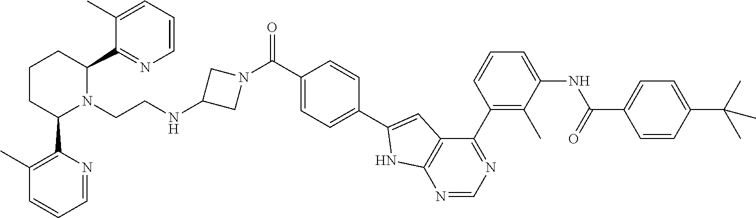 [SMILES: Cc1cccnc1[C@H]1CCC[C@@H](c2ncccc2C)N1CCNC1CN(C(=O)c2ccc(-c3cc4c(-c5cccc(NC(=O)c6ccc(C(C)(C)C)cc6)c5C)ncnc4[nH]3)cc2)C1] |
| I-9 | 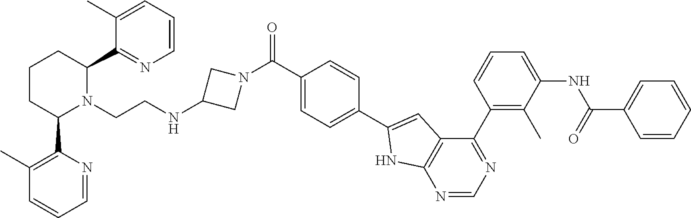 [SMILES: Cc1cccnc1[C@H]1CCC[C@@H](c2ncccc2C)N1CCNC1CN(C(=O)c2ccc(-c3cc4c(-c5cccc(NC(=O)c6ccccc6)c5C)ncnc4[nH]3)cc2)C1] |
| I-10 | 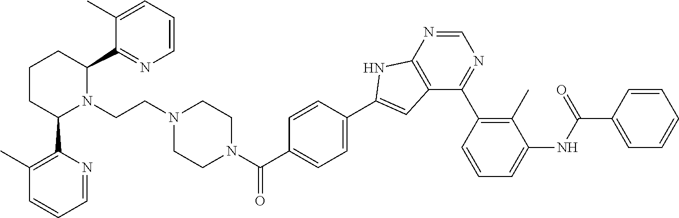 [SMILES: Cc1cccnc1[C@H]1CCC[C@@H](c2ncccc2C)N1CCN1CCN(C(=O)c2ccc(-c3cc4c(-c5cccc(NC(=O)c6ccccc6)c5C)ncnc4[nH]3)cc2)CC1] |
| I-11 | 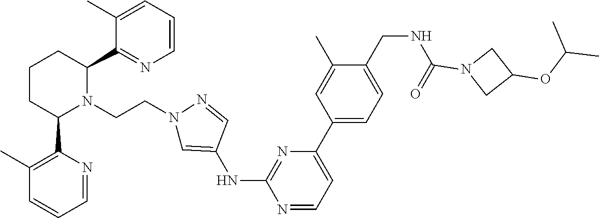 [SMILES: Cc1cc(-c2ccnc(Nc3cnn(CCN4[C@@H](c5ncccc5C)CCC[C@H]4c4ncccc4C)c3)n2)ccc1CNC(=O)N1CC(OC(C)C)C1] |
| I-12 | 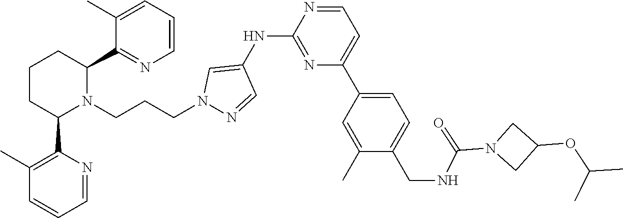 [SMILES: Cc1cc(-c2ccnc(Nc3cnn(CCCN4[C@@H](c5ncccc5C)CCC[C@H]4c4ncccc4C)c3)n2)ccc1CNC(=O)N1CC(OC(C)C)C1] |
| I-13 | 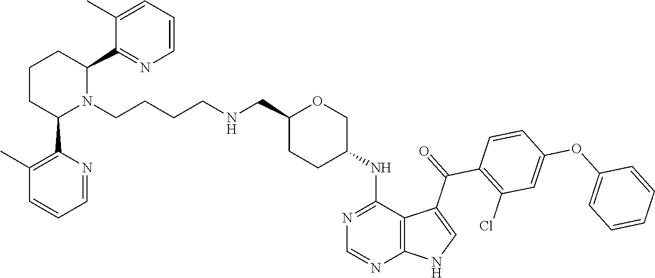 [SMILES: Cc1cccnc1[C@H]1CCC[C@@H](c2ncccc2C)N1CCCCNC[C@@H]1CC[C@@H](Nc2ncnc3[nH]cc(C(=O)c4ccc(Oc5ccccc5)cc4Cl)c23)CO1] |
| I-14 | 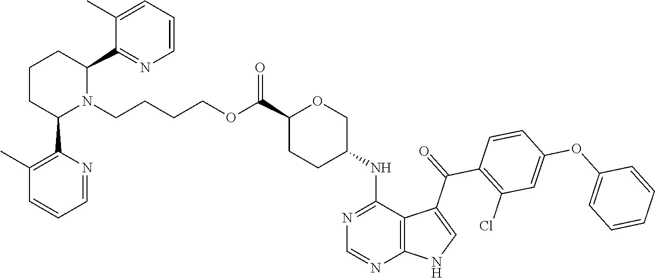 [SMILES: Cc1cccnc1[C@H]1CCC[C@@H](c2ncccc2C)N1CCCCOC(=O)[C@@H]1CC[C@@H](Nc2ncnc3[nH]cc(C(=O)c4ccc(Oc5ccccc5)cc4Cl)c23)CO1] |
| I-15 | 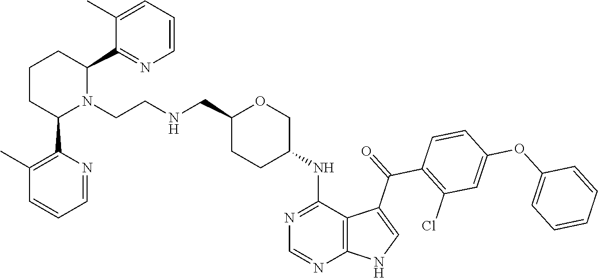 [SMILES: Cc1cccnc1[C@H]1CCC[C@@H](c2ncccc2C)N1CCNC[C@@H]1CC[C@@H](Nc2ncnc3[nH]cc(C(=O)c4ccc(Oc5ccccc5)cc4Cl)c23)CO1] |
| I-16 | 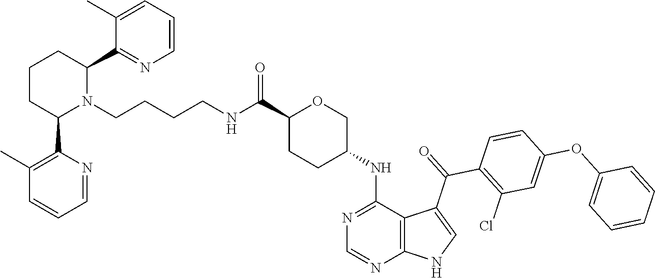 [SMILES: Cc1cccnc1[C@H]1CCC[C@@H](c2ncccc2C)N1CCCCNC(=O)[C@@H]1CC[C@@H](Nc2ncnc3[nH]cc(C(=O)c4ccc(Oc5ccccc5)cc4Cl)c23)CO1] |
| I-17 | 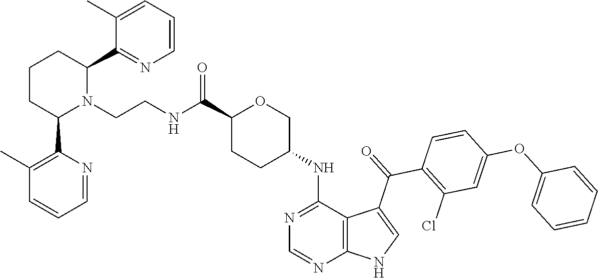 [SMILES: Cc1cccnc1[C@H]1CCC[C@@H](c2ncccc2C)N1CCNC(=O)[C@@H]1CC[C@@H](Nc2ncnc3[nH]cc(C(=O)c4ccc(Oc5ccccc5)cc4Cl)c23)CO1] |
| I-18 | 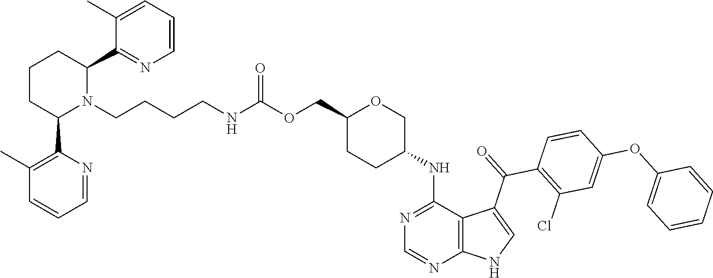 [SMILES: Cc1cccnc1[C@H]1CCC[C@@H](c2ncccc2C)N1CCCCNC(=O)OC[C@@H]1CC[C@@H](Nc2ncnc3[nH]cc(C(=O)c4ccc(Oc5ccccc5)cc4Cl)c23)CO1] |
| I-19 | 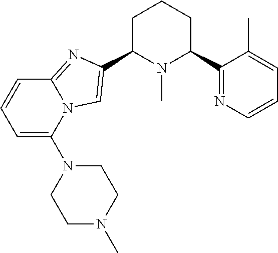 [SMILES: Cc1cccnc1[C@@H]1CCC[C@H](c2cn3c(N4CCN(C)CC4)cccc3n2)N1C] |
| I-20 | 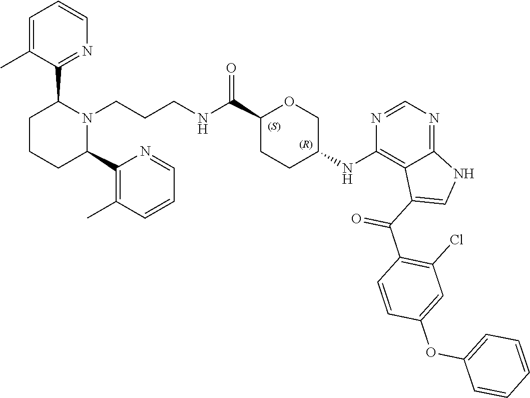 |
| I-21 | 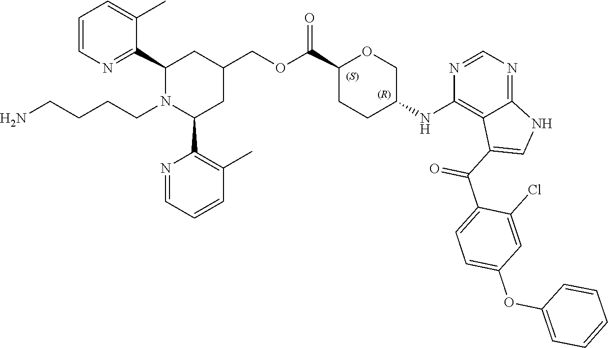 |
| I-22 | 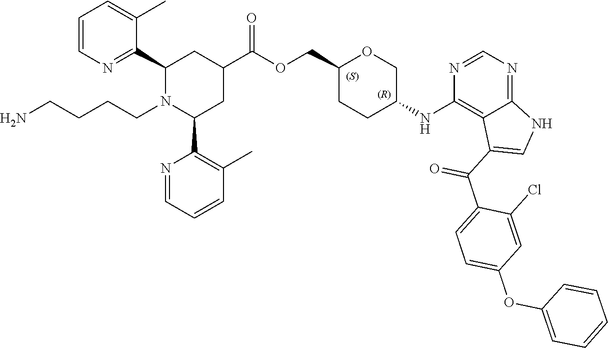 |
| I-23 | 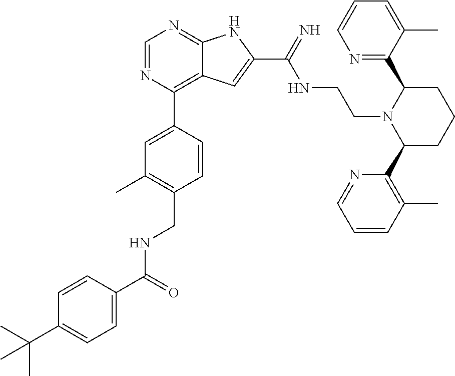 [SMILES: Cc1cc(-c2ncnc3[nH]c(C(=N)NCCN4[C@@H](c5ncccc5C)CCC[C@H]4c4ncccc4C)cc23)ccc1CNC(=O)c1ccc(C(C)(C)C)cc1] |
| I-24 | 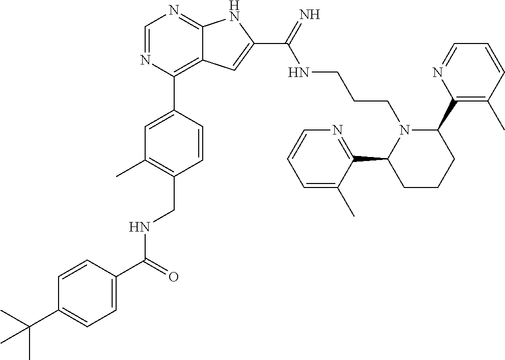 [SMILES: Cc1cc(-c2ncnc3[nH]c(C(=N)NCCCN4[C@@H](c5ncccc5C)CCC[C@H]4c4ncccc4C)cc23)ccc1CNC(=O)c1ccc(C(C)(C)C)cc1] |
| I-25 | 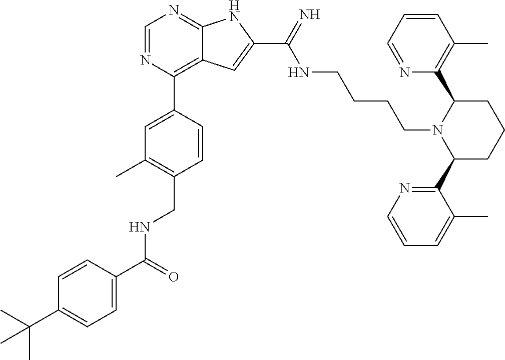 [SMILES: Cc1cc(-c2ncnc3[nH]c(C(=N)NCCCCN4[C@@H](c5ncccc5C)CCC[C@H]4c4ncccc4C)cc23)ccc1CNC(=O)c1ccc(C(C)(C)C)cc1] |
| I-26 | 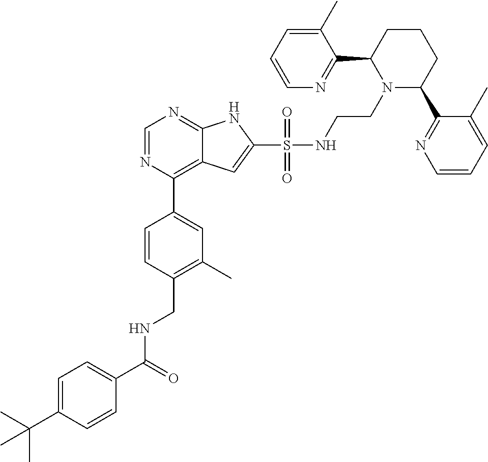 [SMILES: Cc1cc(-c2ncnc3[nH]c(S(=O)(=O)NCCN4[C@@H](c5ncccc5C)CCC[C@H]4c4ncccc4C)cc23)ccc1CNC(=O)c1ccc(C(C)(C)C)cc1] |
| I-27 | 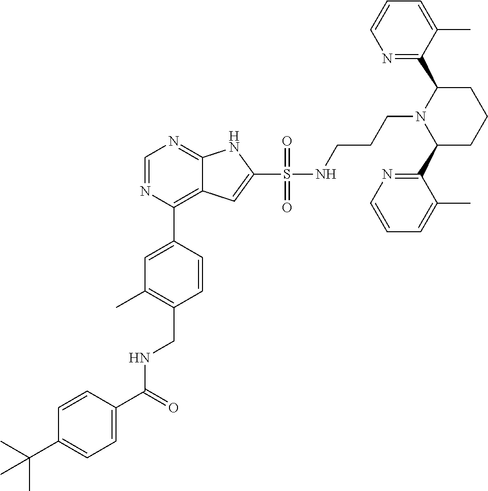 [SMILES: Cc1cc(-c2ncnc3[nH]c(S(=O)(=O)NCCCN4[C@@H](c5ncccc5C)CCC[C@H]4c4ncccc4C)cc23)ccc1CNC(=O)c1ccc(C(C)(C)C)cc1] |
| I-28 | 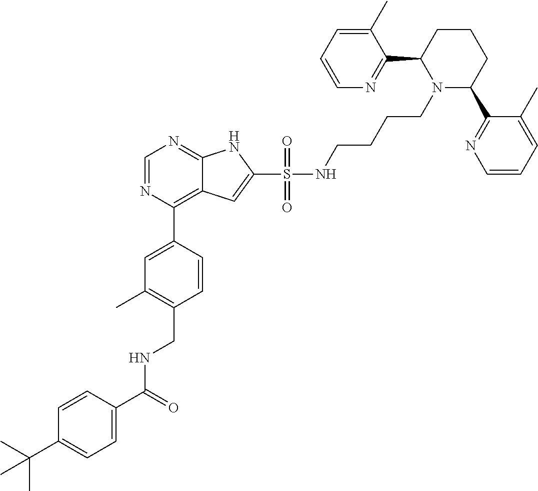 [SMILES: Cc1cc(-c2ncnc3[nH]c(S(=O)(=O)NCCCCN4[C@@H](c5ncccc5C)CCC[C@H]4c4ncccc4C)cc23)ccc1CNC(=O)c1ccc(C(C)(C)C)cc1] |
| I-29 | 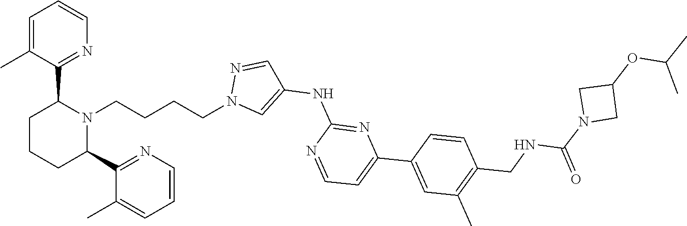 [SMILES: Cc1cc(-c2ccnc(Nc3cnn(CCCCN4[C@@H](c5ncccc5C)CCC[C@H]4c4ncccc4C)c3)n2)ccc1CNC(=O)N1CC(OC(C)C)C1] |
| I-30 | 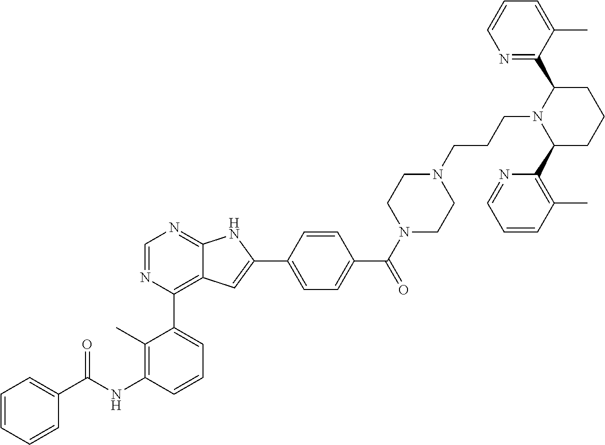 [SMILES: Cc1cccnc1[C@H]1CCC[C@@H](c2ncccc2C)N1CCCN1CCN(C(=O)c2ccc(-c3cc4c(-c5cccc(NC(=O)c6ccccc6)c5C)ncnc4[nH]3)cc2)CC1] |
| I-31 | 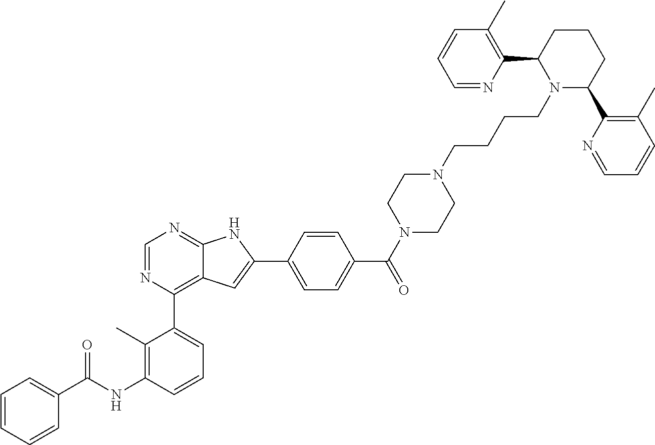 [SMILES: Cc1cccnc1[C@H]1CCC[C@@H](c2ncccc2C)N1CCCCN1CCN(C(=O)c2ccc(-c3cc4c(-c5cccc(NC(=O)c6ccccc6)c5C)ncnc4[nH]3)cc2)CC1] |
| I-32 | 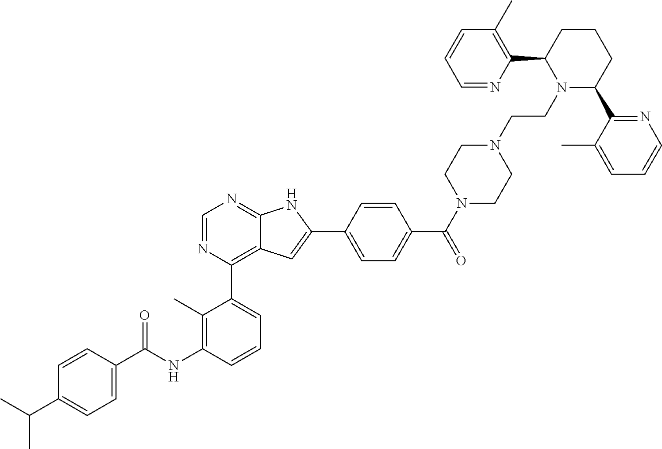 [SMILES: Cc1cccnc1[C@H]1CCC[C@@H](c2ncccc2C)N1CCN1CCN(C(=O)c2ccc(-c3cc4c(-c5cccc(NC(=O)c6ccc(C(C)C)cc6)c5C)ncnc4[nH]3)cc2)CC1] |
| I-33 | 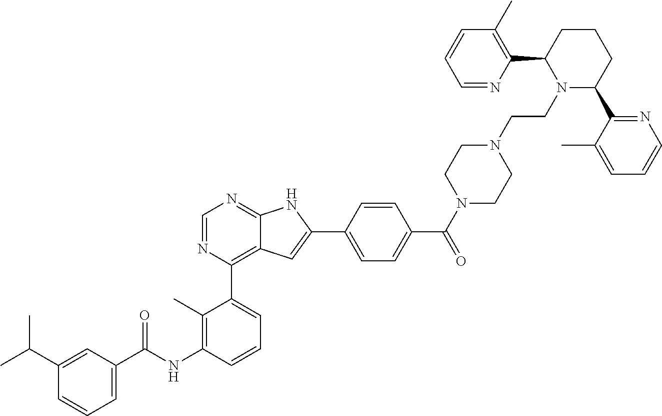 [SMILES: Cc1cccnc1[C@H]1CCC[C@@H](c2ncccc2C)N1CCN1CCN(C(=O)c2ccc(-c3cc4c(-c5cccc(NC(=O)c6cccc(C(C)C)c6)c5C)ncnc4[nH]3)cc2)CC1] |
| I-34 | 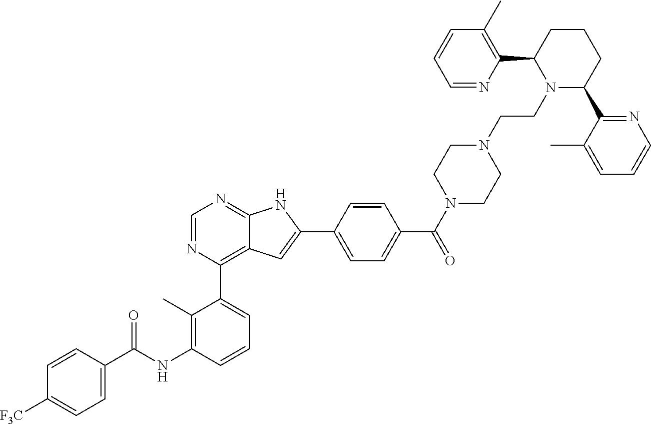 [SMILES: Cc1cccnc1[C@H]1CCC[C@@H](c2ncccc2C)N1CCN1CCN(C(=O)c2ccc(-c3cc4c(-c5cccc(NC(=O)c6ccc(C(F)(F)F)cc6)c5C)ncnc4[nH]3)cc2)CC1] |
| I-35 | 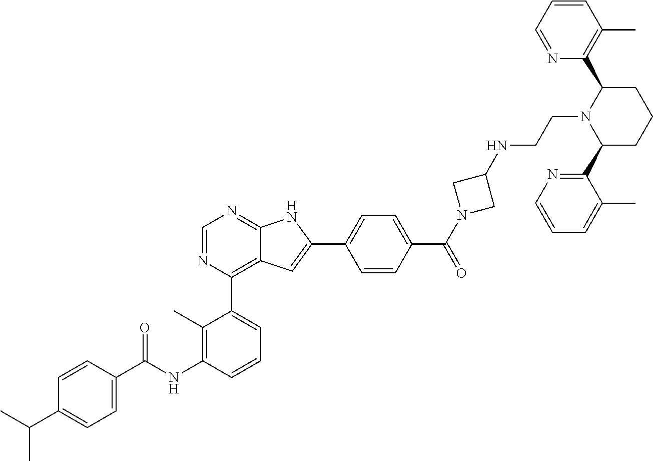 [SMILES: Cc1cccnc1[C@H]1CCC[C@@H](c2ncccc2C)N1CCNC1CN(C(=O)c2ccc(-c3cc4c(-c5cccc(NC(=O)c6ccc(C(C)C)cc6)c5C)ncnc4[nH]3)cc2)C1] |
| I-36 | 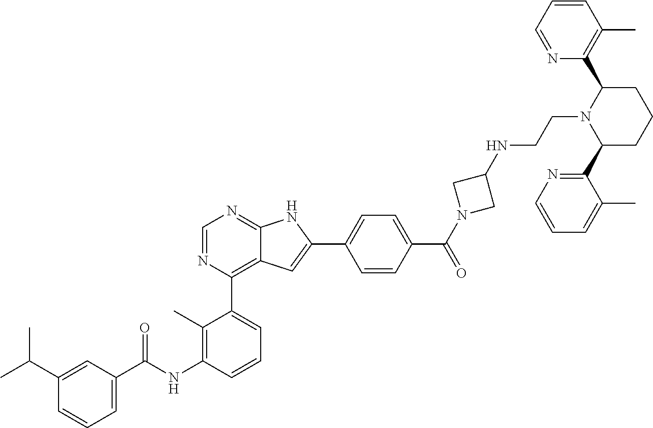 [SMILES: Cc1cccnc1[C@H]1CCC[C@@H](c2ncccc2C)N1CCNC1CN(C(=O)c2ccc(-c3cc4c(-c5cccc(NC(=O)c6cccc(C(C)C)c6)c5C)ncnc4[nH]3)cc2)C1] |
| I-37 | 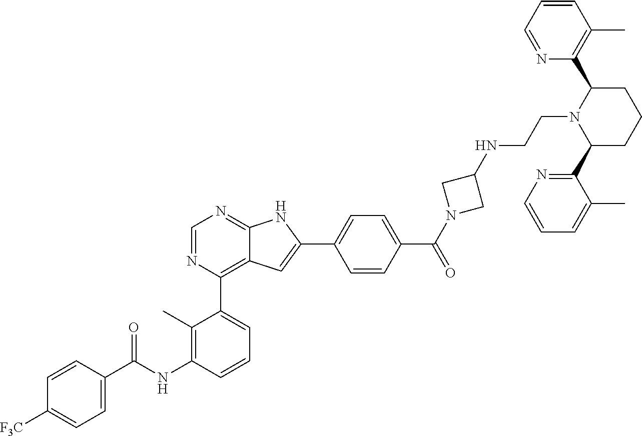 [SMILES: Cc1cccnc1[C@H]1CCC[C@@H](c2ncccc2C)N1CCNC1CN(C(=O)c2ccc(-c3cc4c(-c5cccc(NC(=O)c6ccc(C(F)(F)F)cc6)c5C)ncnc4[nH]3)cc2)C1] |
| I-38 | 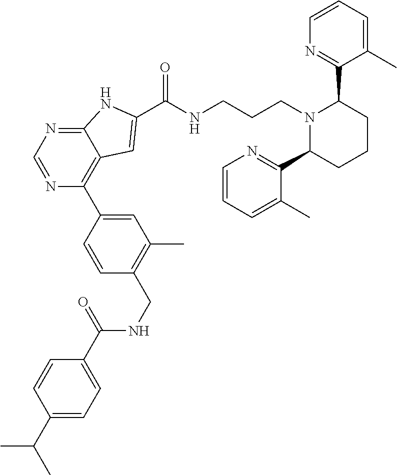 [SMILES: Cc1cc(-c2ncnc3[nH]c(C(=O)NCCCN4[C@@H](c5ncccc5C)CCC[C@H]4c4ncccc4C)cc23)ccc1CNC(=O)c1ccc(C(C)C)cc1] |
| I-39 | 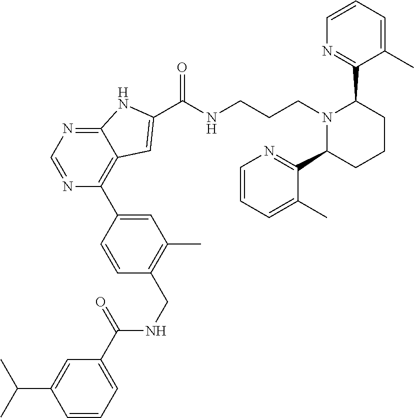 [SMILES: Cc1cc(-c2ncnc3[nH]c(C(=O)NCCCN4[C@@H](c5ncccc5C)CCC[C@H]4c4ncccc4C)cc23)ccc1CNC(=O)c1cccc(C(C)C)c1] |
| I-40 | 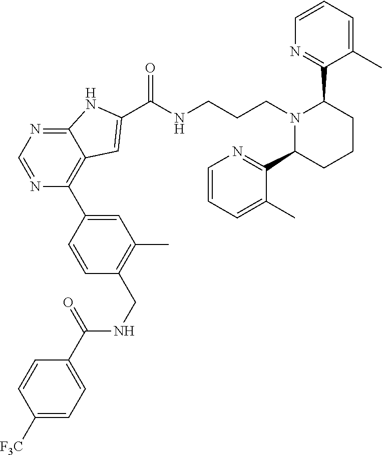 [SMILES: Cc1cc(-c2ncnc3[nH]c(C(=O)NCCCN4[C@@H](c5ncccc5C)CCC[C@H]4c4ncccc4C)cc23)ccc1CNC(=O)c1ccc(C(F)(F)F)cc1] |
| I-41 | 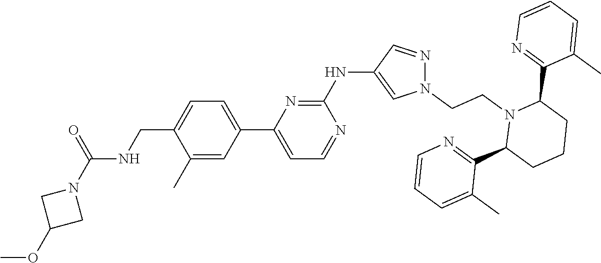 |
| I-42 | 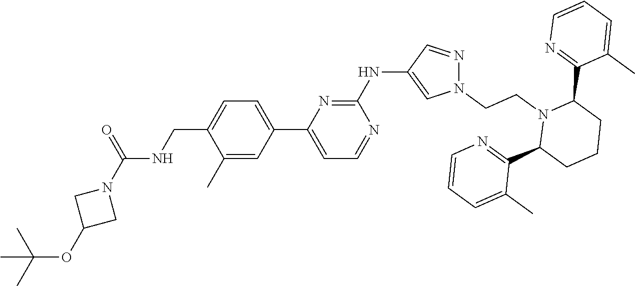 [SMILES: Cc1cc(-c2ccnc(Nc3cnn(CCN4[C@@H](c5ncccc5C)CCC[C@H]4c4ncccc4C)c3)n2)ccc1CNC(=O)N1CC(OC(C)(C)C)C1] |
| I-43 | 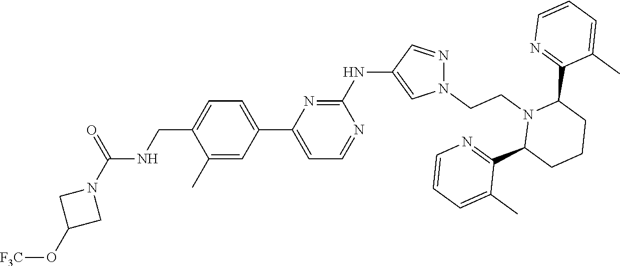 [SMILES: Cc1cc(-c2ccnc(Nc3cnn(CCN4[C@@H](c5ncccc5C)CCC[C@H]4c4ncccc4C)c3)n2)ccc1CNC(=O)N1CC(OC(F)(F)F)C1] |
| I-44 | 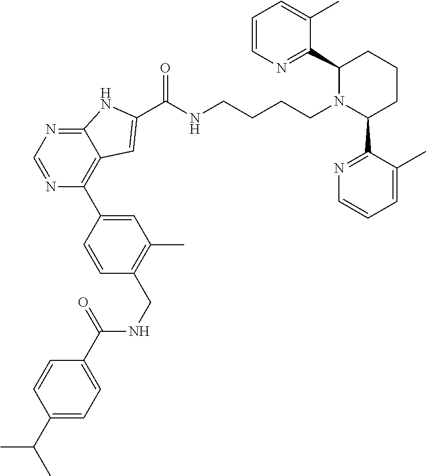 [SMILES: Cc1cc(-c2ncnc3[nH]c(C(=O)NCCCCN4[C@@H](c5ncccc5C)CCC[C@H]4c4ncccc4C)cc23)ccc1CNC(=O)c1ccc(C(C)C)cc1] |
| I-45 | 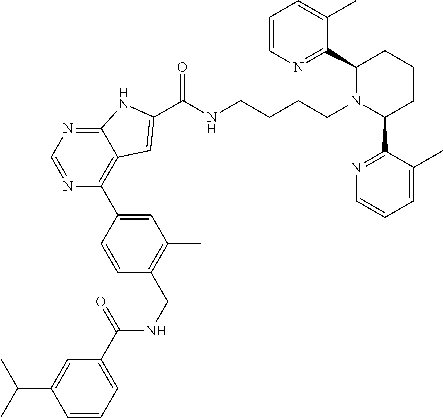 [SMILES: Cc1cc(-c2ncnc3[nH]c(C(=O)NCCCCN4[C@@H](c5ncccc5C)CCC[C@H]4c4ncccc4C)cc23)ccc1CNC(=O)c1cccc(C(C)C)c1] |
| I-46 | 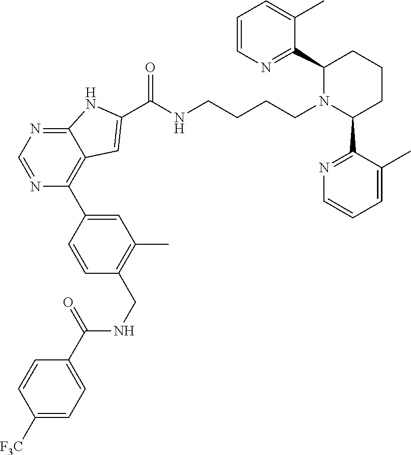 [SMILES: Cc1cc(-c2ncnc3[nH]c(C(=O)NCCCCN4[C@@H](c5ncccc5C)CCC[C@H]4c4ncccc4C)cc23)ccc1CNC(=O)c1ccc(C(F)(F)F)cc1] |
| I-47 | 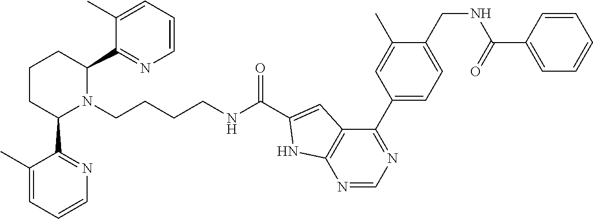 [SMILES: Cc1cc(-c2ncnc3[nH]c(C(=O)NCCCCN4[C@@H](c5ncccc5C)CCC[C@H]4c4ncccc4C)cc23)ccc1CNC(=O)c1ccccc1] |
| I-48 | 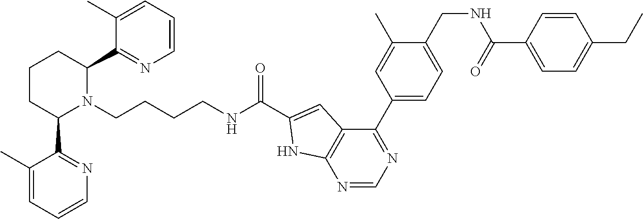 [SMILES: CCc1ccc(C(=O)NCc2ccc(-c3ncnc4[nH]c(C(=O)NCCCCN5[C@@H](c6ncccc6C)CCC[C@H]5c5ncccc5C)cc34)cc2C)cc1] |
| I-49 | 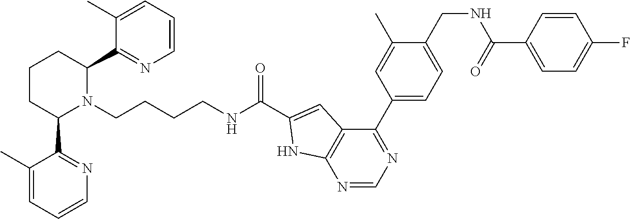 [SMILES: Cc1cc(-c2ncnc3[nH]c(C(=O)NCCCCN4[C@@H](c5ncccc5C)CCC[C@H]4c4ncccc4C)cc23)ccc1CNC(=O)c1ccc(F)cc1] |
| I-50 | 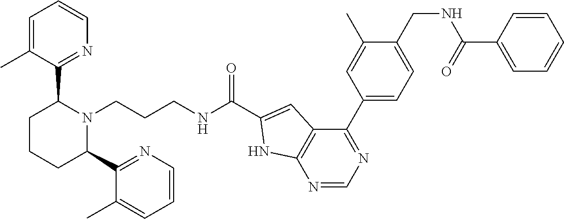 [SMILES: Cc1cc(-c2ncnc3[nH]c(C(=O)NCCCN4[C@@H](c5ncccc5C)CCC[C@H]4c4ncccc4C)cc23)ccc1CNC(=O)c1ccccc1] |
| I-51 | 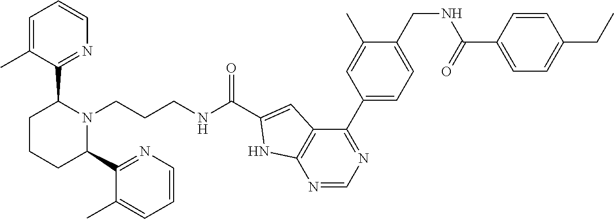 [SMILES: CCc1ccc(C(=O)NCc2ccc(-c3ncnc4[nH]c(C(=O)NCCCN5[C@@H](c6ncccc6C)CCC[C@H]5c5ncccc5C)cc34)cc2C)cc1] |
| I-52 | 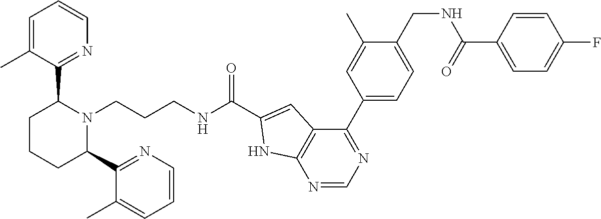 [SMILES: Cc1cc(-c2ncnc3[nH]c(C(=O)NCCCN4[C@@H](c5ncccc5C)CCC[C@H]4c4ncccc4C)cc23)ccc1CNC(=O)c1ccc(F)cc1] |
| I-53 | 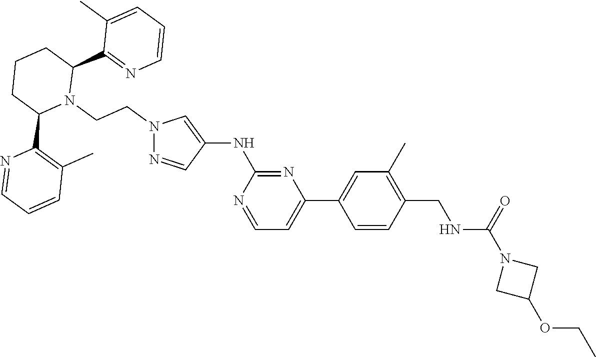 [SMILES: CCOC1CN(C(=O)NCc2ccc(-c3ccnc(Nc4cnn(CCN5[C@@H](c6ncccc6C)CCC[C@H]5c5ncccc5C)c4)n3)cc2C)C1] |
| I-54 | 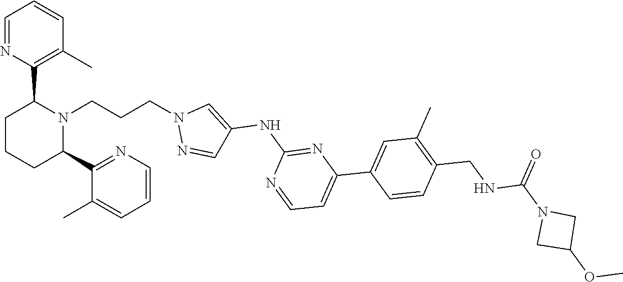 [SMILES: COC1CN(C(=O)NCc2ccc(-c3ccnc(Nc4cnn(CCCN5[C@@H](c6ncccc6C)CCC[C@H]5c5ncccc5C)c4)n3)cc2C)C1] |
| I-55 | 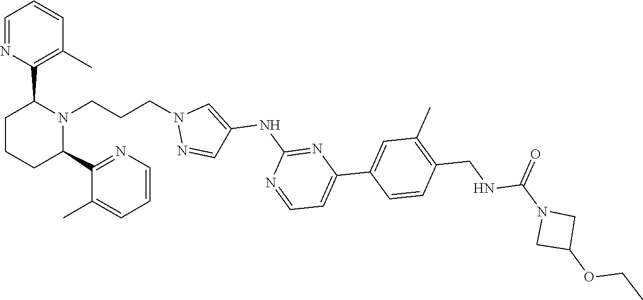 [SMILES: CCOC1CN(C(=O)NCc2ccc(-c3ccnc(Nc4cnn(CCCN5[C@@H](c6ncccc6C)CCC[C@H]5c5ncccc5C)c4)n3)cc2C)C1] |
| I-56 | 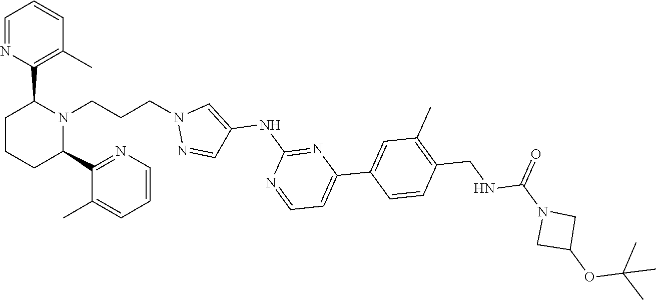 [SMILES: Cc1cc(-c2ccnc(Nc3cnn(CCCN4[C@@H](c5ncccc5C)CCC[C@H]4c4ncccc4C)c3)n2)ccc1CNC(=O)N1CC(OC(C)(C)C)C1] |
| I-57 | 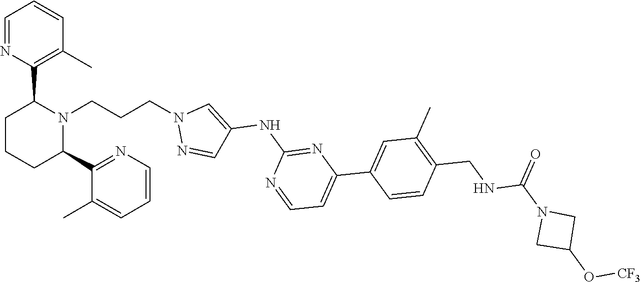 [SMILES: Cc1cc(-c2ccnc(Nc3cnn(CCCN4[C@@H](c5ncccc5C)CCC[C@H]4c4ncccc4C)c3)n2)ccc1CNC(=O)N1CC(OC(F)(F)F)C1] |
| I-58 | 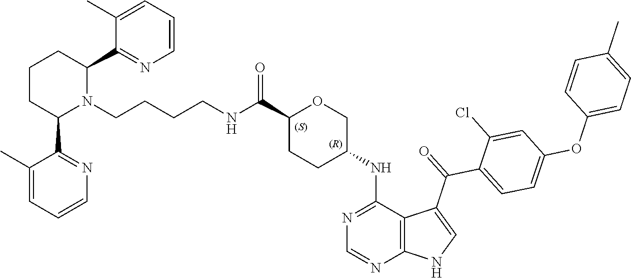 |
| I-59 | 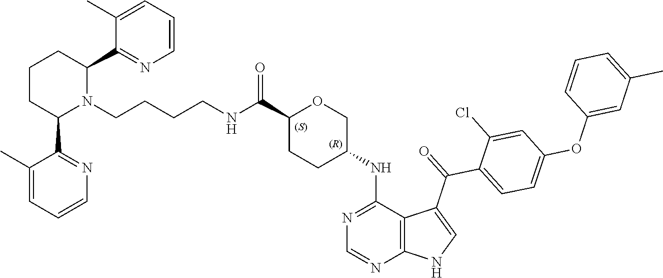 |
| I-60 | 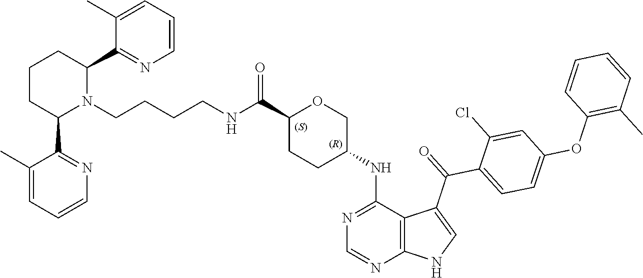 |
| I-61 | 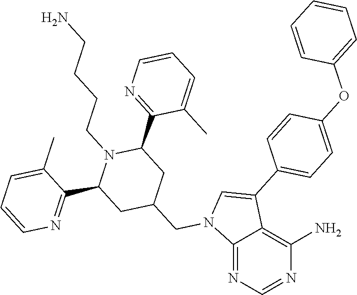 [SMILES: Cc1cccnc1[C@H]1CC(Cn2cc(-c3ccc(Oc4ccccc4)cc3)c3c(N)ncnc32)C[C@@H](c2ncccc2C)N1CCCCN] |
| I-62 | 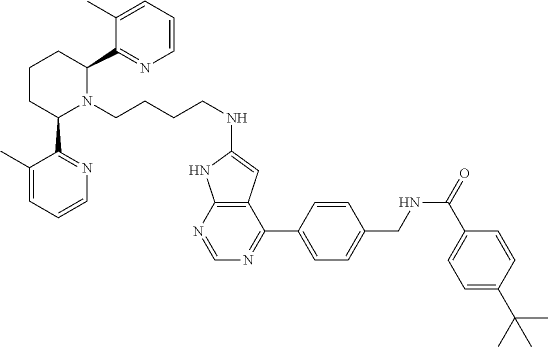 [SMILES: Cc1cccnc1[C@H]1CCC[C@@H](c2ncccc2C)N1CCCCNc1cc2c(-c3ccc(CNC(=O)c4ccc(C(C)(C)C)cc4)cc3)ncnc2[nH]1] |
| I-63 | 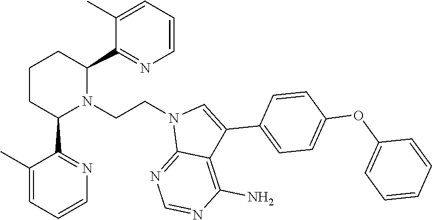 [SMILES: Cc1cccnc1[C@H]1CCC[C@@H](c2ncccc2C)N1CCn1cc(-c2ccc(Oc3ccccc3)cc2)c2c(N)ncnc21] |
| I-64 | 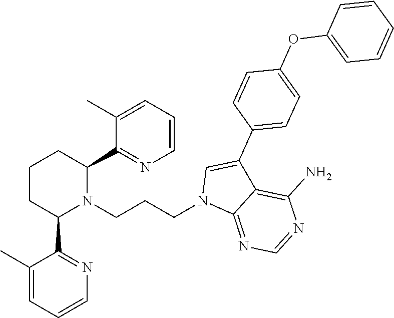 [SMILES: Cc1cccnc1[C@H]1CCC[C@@H](c2ncccc2C)N1CCCn1cc(-c2ccc(Oc3ccccc3)cc2)c2c(N)ncnc21] |
| I-65 | 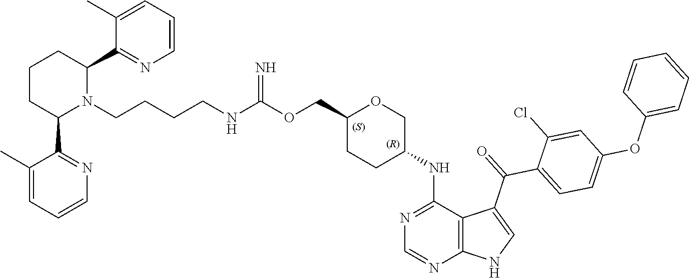 |
| I-66 | 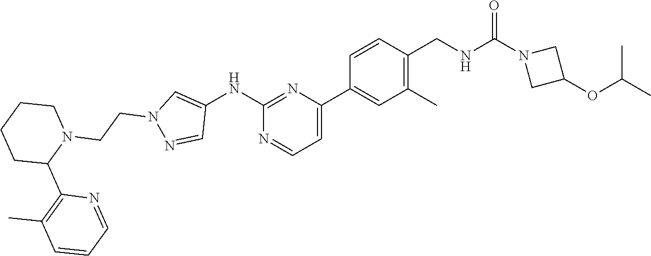 |
| I-67 | 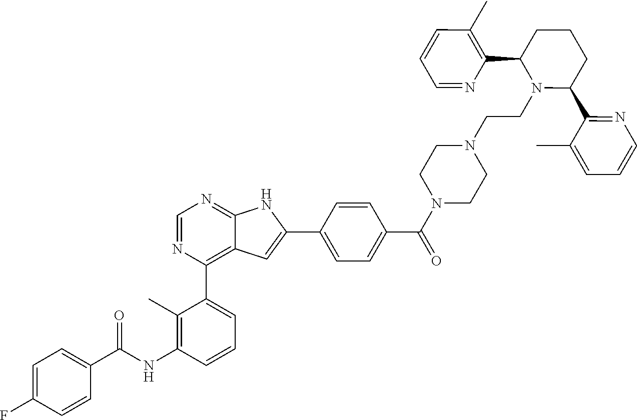 [SMILES: Cc1cccnc1[C@H]1CCC[C@@H](c2ncccc2C)N1CCN1CCN(C(=O)c2ccc(-c3cc4c(-c5cccc(NC(=O)c6ccc(F)cc6)c5C)ncnc4[nH]3)cc2)CC1] |
| I-68 | 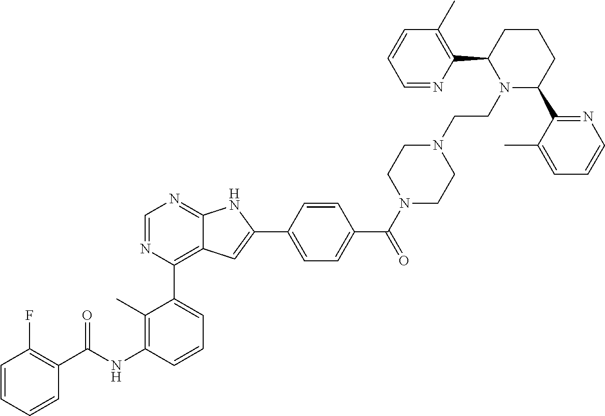 [SMILES: Cc1cccnc1[C@H]1CCC[C@@H](c2ncccc2C)N1CCN1CCN(C(=O)c2ccc(-c3cc4c(-c5cccc(NC(=O)c6ccccc6F)c5C)ncnc4[nH]3)cc2)CC1] |
| I-69 | 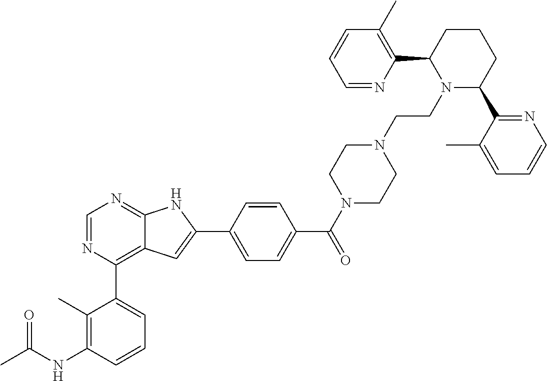 [SMILES: CC(=O)Nc1cccc(-c2ncnc3[nH]c(-c4ccc(C(=O)N5CCN(CCN6[C@@H](c7ncccc7C)CCC[C@H]6c6ncccc6C)CC5)cc4)cc23)c1C] |
| I-70 | 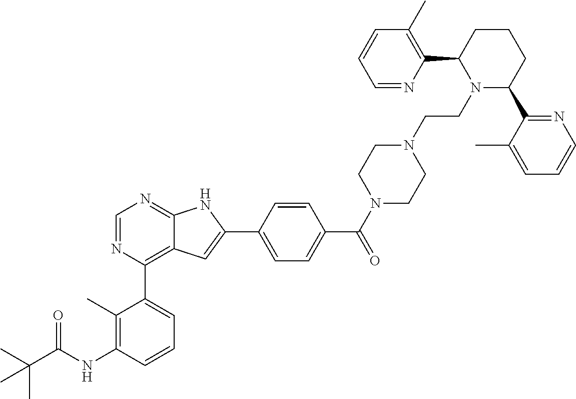 [SMILES: Cc1cccnc1[C@H]1CCC[C@@H](c2ncccc2C)N1CCN1CCN(C(=O)c2ccc(-c3cc4c(-c5cccc(NC(=O)C(C)(C)C)c5C)ncnc4[nH]3)cc2)CC1] |
| I-71 | 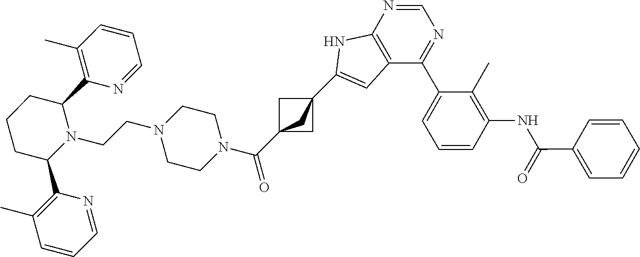 [SMILES: Cc1cccnc1[C@H]1CCC[C@@H](c2ncccc2C)N1CCN1CCN(C(=O)[C@]23C[C@](c4cc5c(-c6cccc(NC(=O)c7ccccc7)c6C)ncnc5[nH]4)(C2)C3)CC1] |
| I-72 | 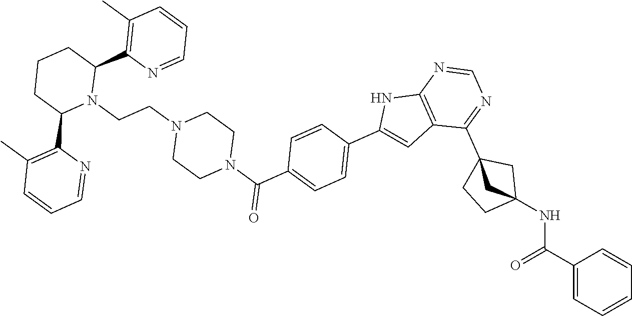 [SMILES: Cc1cccnc1[C@H]1CCC[C@@H](c2ncccc2C)N1CCN1CCN(C(=O)c2ccc(-c3cc4c(ncnc4[C@]45CC[C@](NC(=O)c6ccccc6)(C4)C5)[nH]3)cc2)CC1] |
| I-73 | 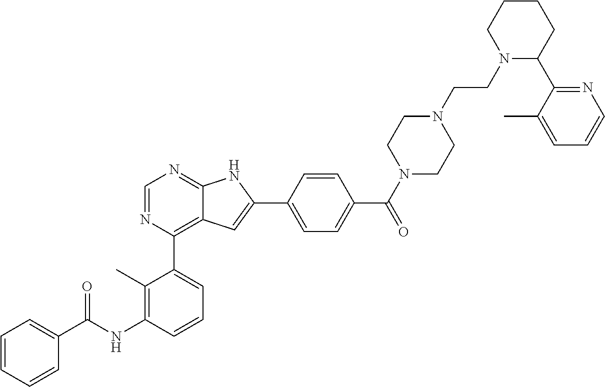 [SMILES: Cc1cccnc1C1CCCCN1CCN1CCN(C(=O)c2ccc(-c3cc4c(-c5cccc(NC(=O)c6ccccc6)c5C)ncnc4[nH]3)cc2)CC1] |
| I-74 | 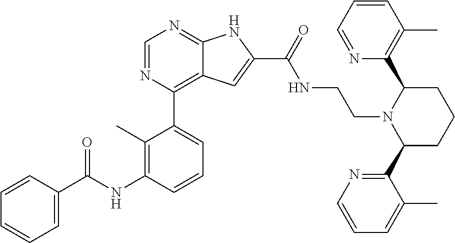 [SMILES: Cc1cccnc1[C@H]1CCC[C@@H](c2ncccc2C)N1CCNC(=O)c1cc2c(-c3cccc(NC(=O)c4ccccc4)c3C)ncnc2[nH]1] |
| I-75 | 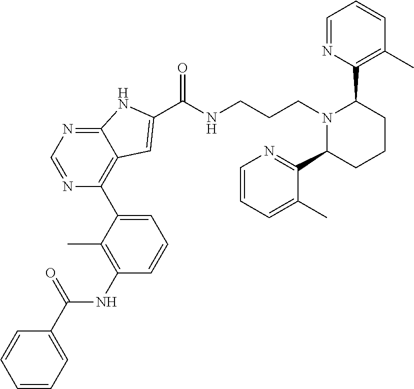 [SMILES: Cc1cccnc1[C@H]1CCC[C@@H](c2ncccc2C)N1CCCNC(=O)c1cc2c(-c3cccc(NC(=O)c4ccccc4)c3C)ncnc2[nH]1] |
| I-76 | 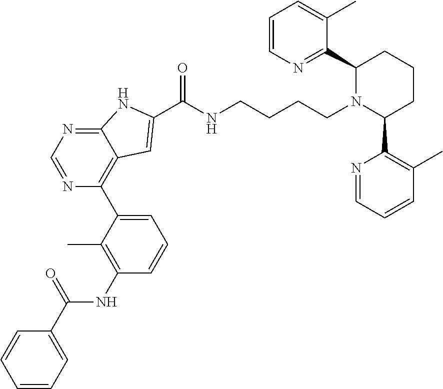 [SMILES: Cc1cccnc1[C@H]1CCC[C@@H](c2ncccc2C)N1CCCCNC(=O)c1cc2c(-c3cccc(NC(=O)c4ccccc4)c3C)ncnc2[nH]1] |
| I-77 | 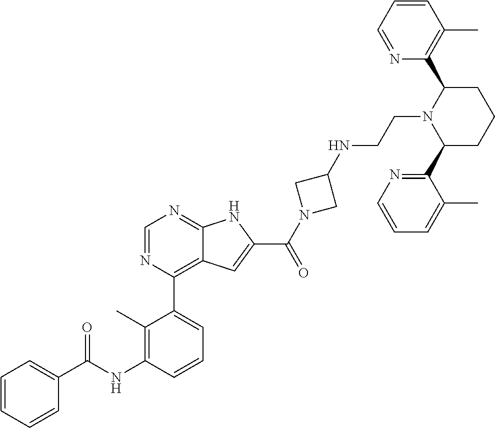 [SMILES: Cc1cccnc1[C@H]1CCC[C@@H](c2ncccc2C)N1CCNC1CN(C(=O)c2cc3c(-c4cccc(NC(=O)c5ccccc5)c4C)ncnc3[nH]2)C1] |
| I-78 | 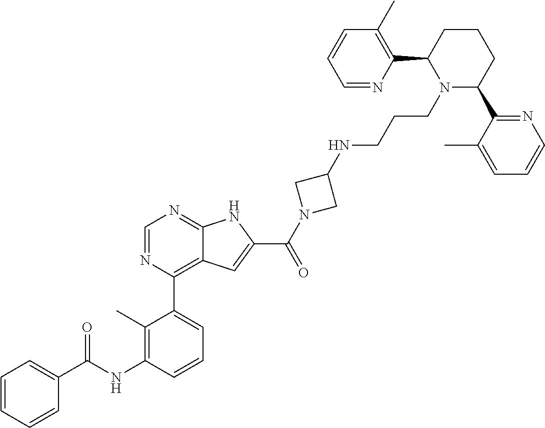 [SMILES: Cc1cccnc1[C@H]1CCC[C@@H](c2ncccc2C)N1CCCNC1CN(C(=O)c2cc3c(-c4cccc(NC(=O)c5ccccc5)c4C)ncnc3[nH]2)C1] |
| I-79 | 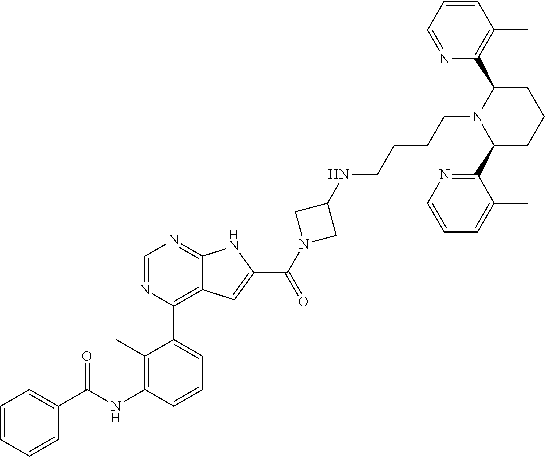 [SMILES: Cc1cccnc1[C@H]1CCC[C@@H](c2ncccc2C)N1CCCCNC1CN(C(=O)c2cc3c(-c4cccc(NC(=O)c5ccccc5)c4C)ncnc3[nH]2)C1] |
| I-80 | 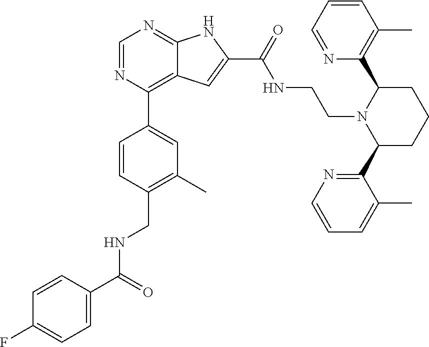 [SMILES: Cc1cc(-c2ncnc3[nH]c(C(=O)NCCN4[C@@H](c5ncccc5C)CCC[C@H]4c4ncccc4C)cc23)ccc1CNC(=O)c1ccc(F)cc1] |
| I-81 | 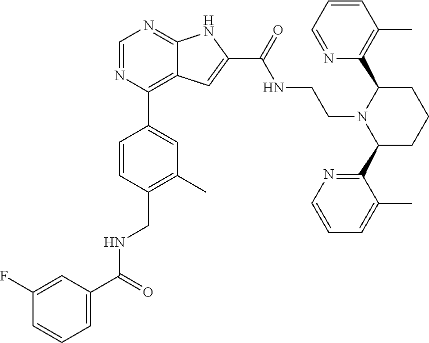 [SMILES: Cc1cc(-c2ncnc3[nH]c(C(=O)NCCN4[C@@H](c5ncccc5C)CCC[C@H]4c4ncccc4C)cc23)ccc1CNC(=O)c1cccc(F)c1] |
| I-82 | 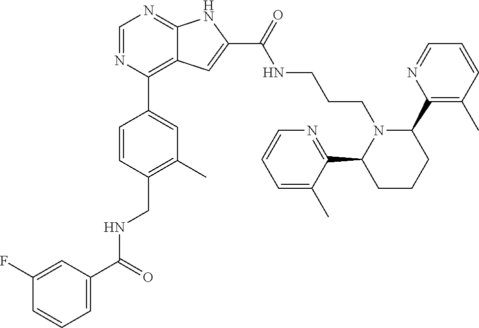 [SMILES: Cc1cc(-c2ncnc3[nH]c(C(=O)NCCCN4[C@@H](c5ncccc5C)CCC[C@H]4c4ncccc4C)cc23)ccc1CNC(=O)c1cccc(F)c1] |
| I-83 | 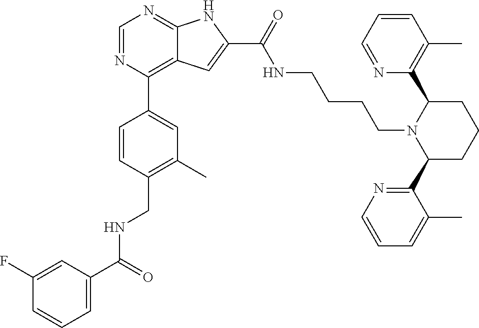 [SMILES: Cc1cc(-c2ncnc3[nH]c(C(=O)NCCCCN4[C@@H](c5ncccc5C)CCC[C@H]4c4ncccc4C)cc23)ccc1CNC(=O)c1cccc(F)c1] |
| I-84 | 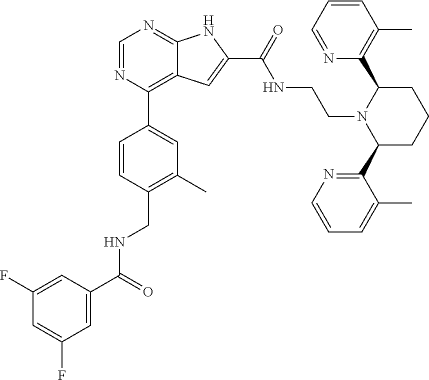 [SMILES: Cc1cc(-c2ncnc3[nH]c(C(=O)NCCN4[C@@H](c5ncccc5C)CCC[C@H]4c4ncccc4C)cc23)ccc1CNC(=O)c1cc(F)cc(F)c1] |
| I-85 | 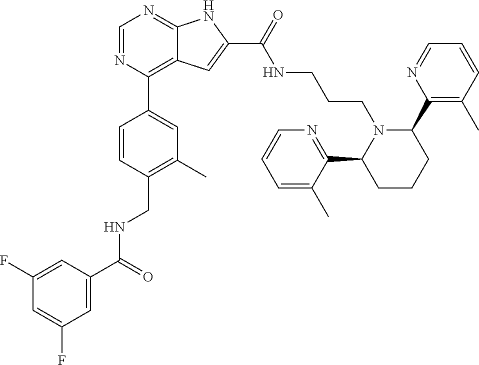 [SMILES: Cc1cc(-c2ncnc3[nH]c(C(=O)NCCCN4[C@@H](c5ncccc5C)CCC[C@H]4c4ncccc4C)cc23)ccc1CNC(=O)c1cc(F)cc(F)c1] |
| I-86 | 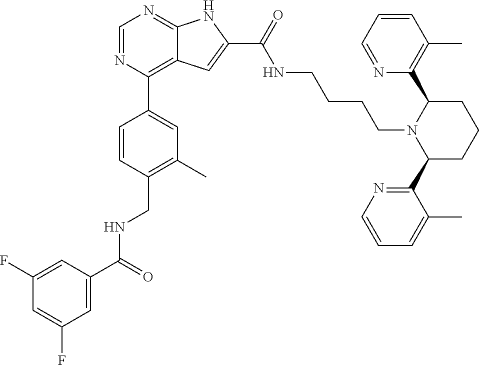 [SMILES: Cc1cc(-c2ncnc3[nH]c(C(=O)NCCCCN4[C@@H](c5ncccc5C)CCC[C@H]4c4ncccc4C)cc23)ccc1CNC(=O)c1cc(F)cc(F)c1] |
| I-87 | 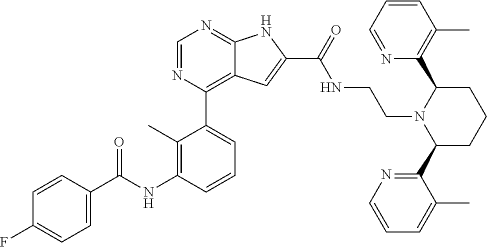 [SMILES: Cc1cccnc1[C@H]1CCC[C@@H](c2ncccc2C)N1CCNC(=O)c1cc2c(-c3cccc(NC(=O)c4ccc(F)cc4)c3C)ncnc2[nH]1] |
| I-88 | 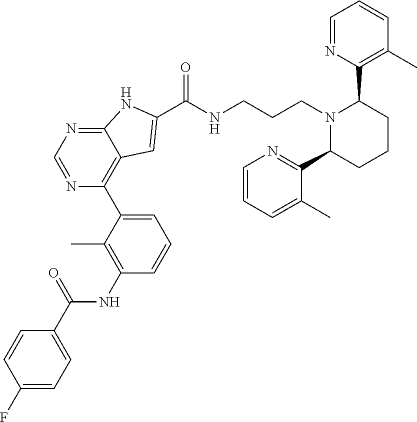 [SMILES: Cc1cccnc1[C@H]1CCC[C@@H](c2ncccc2C)N1CCCNC(=O)c1cc2c(-c3cccc(NC(=O)c4ccc(F)cc4)c3C)ncnc2[nH]1] |
| I-89 | 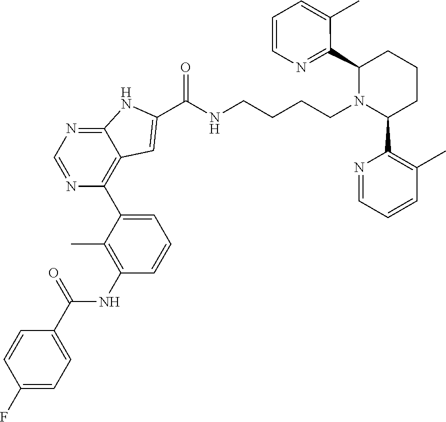 [SMILES: Cc1cccnc1[C@H]1CCC[C@@H](c2ncccc2C)N1CCCCNC(=O)c1cc2c(-c3cccc(NC(=O)c4ccc(F)cc4)c3C)ncnc2[nH]1] |
| I-90 | 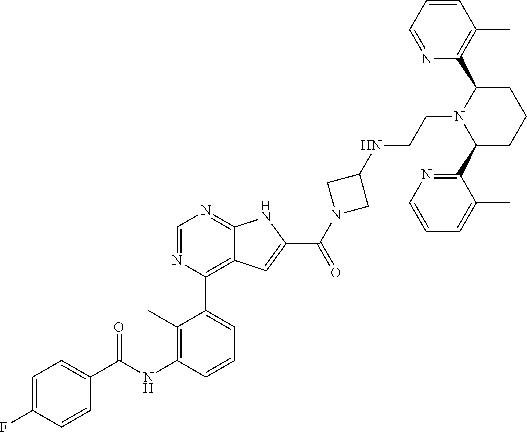 [SMILES: Cc1cccnc1[C@H]1CCC[C@@H](c2ncccc2C)N1CCNC1CN(C(=O)c2cc3c(-c4cccc(NC(=O)c5ccc(F)cc5)c4C)ncnc3[nH]2)C1] |
| I-91 | 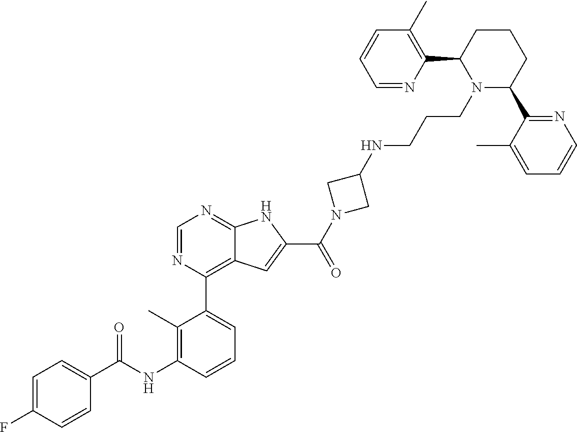 [SMILES: Cc1cccnc1[C@H]1CCC[C@@H](c2ncccc2C)N1CCCNC1CN(C(=O)c2cc3c(-c4cccc(NC(=O)c5ccc(F)cc5)c4C)ncnc3[nH]2)C1] |
| I-92 | 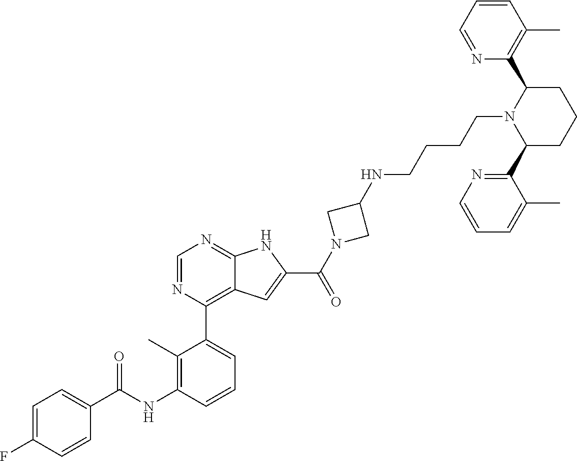 [SMILES: Cc1cccnc1[C@H]1CCC[C@@H](c2ncccc2C)N1CCCCNC1CN(C(=O)c2cc3c(-c4cccc(NC(=O)c5ccc(F)cc5)c4C)ncnc3[nH]2)C1] |

Treatment of Diseases, Disorders, and Conditions Associated with CXCR4 and/or BTK

Compounds provided by the present invention are useful in treating a variety of diseases, disorders, and conditions associated with CXCR4 and/or BTK. For example, in some embodiments, the dual inhibitor compounds of the invention give surprising and unexpected activity in the form of a 10-fold improvement compared to simply combining the two single activity agents (a CXCR4 inhibitor and a BTK inhibitor) and a 40-fold improvement compared to a selective BTK inhibitor.

In one aspect, the present invention provides a method of treating a disease, disorder, or condition associated with or mediated by CXCR4 and/or BTK, comprising administering to a subject in need thereof an effective amount of a disclosed compound or a pharmaceutically acceptable salt thereof. In some embodiments, the disease, disorder, or condition is associated with aberrant expression of CXCR4 or a mutation in CXCR4. In some embodiments, the disease, disorder, or condition is associated with aberrant expression of BTK or a mutation in BTK.

In some embodiments, the disease, disorder, or condition is a cellular proliferative disorder. In some embodiments, the disease, disorder, or condition is cancer.

Cancer includes, in one embodiment, without limitation, leukemias (e.g., acute leukemia, acute lymphocytic leukemia, acute myelocytic leukemia, acute myeloblastic leukemia, acute promyelocytic leukemia, acute myelomonocytic leukemia, acute monocytic leukemia, acute erythroleukemia, chronic leukemia, chronic myelocytic leukemia, chronic lymphocytic leukemia), polycythemia vera, lymphoma (e.g., Hodgkin's disease or non-Hodgkin's disease), Waldenstrom's macroglobulinemia, multiple myeloma, heavy chain disease, and solid tumors such as sarcomas and carcinomas (e.g., fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilm's tumor, cervical cancer, uterine cancer, testicular cancer, lung carcinoma, small cell lung carcinoma, bladder carcinoma, epithelial carcinoma, glioma, astrocytoma, glioblastoma multiforme (GBM, also known as glioblastoma), medulloblastoma, craniopharyngioma, ependymoma, pinealoma, hemangioblastoma, acoustic neuroma, oligodendroglioma, schwannoma, neurofibrosarcoma, meningioma, melanoma, neuroblastoma, and retinoblastoma).

In some embodiments, the cancer is glioma, astrocytoma, glioblastoma multiforme (GBM, also known as glioblastoma), medulloblastoma, craniopharyngioma, ependymoma, pinealoma, hemangioblastoma, acoustic neuroma, oligodendroglioma, schwannoma, neurofibrosarcoma, meningioma, melanoma, neuroblastoma, or retinoblastoma.

In some embodiments, the cancer is acoustic neuroma, astrocytoma (e.g. Grade I-Pilocytic Astrocytoma, Grade II-Low-grade Astrocytoma, Grade III-Anaplastic Astrocytoma, or Grade IV-Glioblastoma (GBM)), chordoma, CNS lymphoma, craniopharyngioma, brain stem glioma, ependymoma, mixed glioma, optic nerve glioma, subependymoma, medulloblastoma, meningioma, metastatic brain tumor, oligodendroglioma, pituitary tumors, primitive neuroectodermal (PNET) tumor, or schwannoma. In some embodiments, the cancer is a type found more commonly in children than adults, such as brain stem glioma, craniopharyngioma, ependymoma, juvenile pilocytic astrocytoma (JPA), medulloblastoma, optic nerve glioma, pineal tumor, primitive neuroectodermal tumors (PNET), or rhabdoid tumor.

In some embodiments, the patient is an adult human. In some embodiments, the patient is a child or pediatric patient.

Cancer includes, in another embodiment, without limitation, mesothelioma, hepatobiliary (hepatic and biliary duct), bone cancer, pancreatic cancer, skin cancer, cancer of the head or neck, cutaneous or intraocular melanoma, ovarian cancer, colon cancer, rectal cancer, cancer of the anal region, stomach cancer, gastrointestinal (gastric, colorectal, and duodenal), uterine cancer, carcinoma of the fallopian tubes, carcinoma of the endometrium, carcinoma of the cervix, carcinoma of the vagina, carcinoma of the vulva, Hodgkin's Disease, cancer of the esophagus, cancer of the small intestine, cancer of the endocrine system, cancer of the thyroid gland, cancer of the parathyroid gland, cancer of the adrenal gland, sarcoma of soft tissue, cancer of the urethra, cancer of the penis, prostate cancer, testicular cancer, chronic or acute leukemia, chronic myeloid leukemia, lymphocytic lymphomas, cancer of the bladder, cancer of the kidney or ureter, renal cell carcinoma (RCC), carcinoma of the renal pelvis, non-Hodgkin's lymphoma, spinal axis tumors, brain stem glioma, pituitary adenoma, adrenocortical cancer, gall bladder cancer, multiple myeloma, cholangiocarcinoma, fibrosarcoma, neuroblastoma, retinoblastoma, or a combination of one or more of the foregoing cancers.

In some embodiments, the cancer is selected from hepatocellular carcinoma (HCC), ovarian cancer, ovarian epithelial cancer, or fallopian tube cancer; papillary serous cystadenocarcinoma or uterine papillary serous carcinoma (UPSC); prostate cancer; testicular cancer; gallbladder cancer; hepatocholangiocarcinoma; soft tissue and bone synovial sarcoma; rhabdomyosarcoma; osteosarcoma; chondrosarcoma; Ewing sarcoma; anaplastic thyroid cancer; adrenocortical adenoma; pancreatic cancer; pancreatic ductal carcinoma or pancreatic adenocarcinoma; gastrointestinal/stomach (GIST) cancer; lymphoma; squamous cell carcinoma of the head and neck (SCCHN); salivary gland cancer; glioma, or brain cancer; neurofibromatosis-1 associated malignant peripheral nerve sheath tumors (MPNST); Waldenstrom's macroglobulinemia; or medulloblastoma.

In some embodiments, the cancer is selected from hepatocellular carcinoma (HCC), hepatoblastoma, colon cancer, rectal cancer, ovarian cancer, ovarian epithelial cancer, fallopian tube cancer, papillary serous cystadenocarcinoma, uterine papillary serous carcinoma (UPSC), hepatocholangiocarcinoma, soft tissue and bone synovial sarcoma, rhabdomyosarcoma, osteosarcoma, anaplastic thyroid cancer, adrenocortical adenoma, pancreatic cancer, pancreatic ductal carcinoma, pancreatic adenocarcinoma, glioma, neurofibromatosis-1 associated malignant peripheral nerve sheath tumors (MPNST), Waldenstrom's macroglobulinemia, or medulloblastoma.

In some embodiments, the present invention provides a method for treating a cancer that presents as a solid tumor, such as a sarcoma, carcinoma, or lymphoma, comprising the step of administering a disclosed compound, or a pharmaceutically acceptable salt thereof, to a patient in need thereof. Solid tumors generally comprise an abnormal mass of tissue that typically does not include cysts or liquid areas. In some embodiments, the cancer is selected from renal cell carcinoma, or kidney cancer; hepatocellular carcinoma (HCC) or hepatoblastoma, or liver cancer; melanoma; breast cancer; colorectal carcinoma, or colorectal cancer; colon cancer; rectal cancer; anal cancer; lung cancer, such as non-small cell lung cancer (NSCLC) or small cell lung cancer (SCLC); ovarian cancer, ovarian epithelial cancer, ovarian carcinoma, or fallopian tube cancer; papillary serous cystadenocarcinoma or uterine papillary serous carcinoma (UPSC); prostate cancer; testicular cancer; gallbladder cancer; hepatocholangiocarcinoma; soft tissue and bone synovial sarcoma; rhabdomyosarcoma; osteosarcoma; chondrosarcoma; Ewing sarcoma; anaplastic thyroid cancer; adrenocortical carcinoma; pancreatic cancer; pancreatic ductal carcinoma or pancreatic adenocarcinoma; gastrointestinal/stomach (GIST) cancer; lymphoma; squamous cell carcinoma of the head and neck (SCCHN); salivary gland cancer; glioma, or brain cancer; neurofibromatosis-1 associated malignant peripheral nerve sheath tumors (MPNST); Waldenstrom's macroglobulinemia; or medulloblastoma.

In some embodiments, the cancer is selected from renal cell carcinoma, hepatocellular carcinoma (HCC), hepatoblastoma, colorectal carcinoma, colorectal cancer, colon cancer, rectal cancer, anal cancer, ovarian cancer, ovarian epithelial cancer, ovarian carcinoma, fallopian tube cancer, papillary serous cystadenocarcinoma, uterine papillary serous carcinoma (UPSC), hepatocholangiocarcinoma, soft tissue and bone synovial sarcoma, rhabdomyosarcoma, osteosarcoma, chondrosarcoma, anaplastic thyroid cancer, adrenocortical carcinoma, pancreatic cancer, pancreatic ductal carcinoma, pancreatic adenocarcinoma, glioma, brain cancer, neurofibromatosis-1 associated malignant peripheral nerve sheath tumors (MPNST), Waldenstrom's macroglobulinemia, or medulloblastoma.

In some embodiments, the cancer is hepatocellular carcinoma (HCC). In some embodiments, the cancer is hepatoblastoma. In some embodiments, the cancer is colon cancer. In some embodiments, the cancer is rectal cancer. In some embodiments, the cancer is ovarian cancer, or ovarian carcinoma. In some embodiments, the cancer is ovarian epithelial cancer. In some embodiments, the cancer is fallopian tube cancer. In some embodiments, the cancer is papillary serous cystadenocarcinoma. In some embodiments, the cancer is uterine papillary serous carcinoma (UPSC). In some embodiments, the cancer is hepatocholangiocarcinoma. In some embodiments, the cancer is soft tissue and bone synovial sarcoma. In some embodiments, the cancer is rhabdomyosarcoma. In some embodiments, the cancer is osteosarcoma. In some embodiments, the cancer is anaplastic thyroid cancer. In some embodiments, the cancer is adrenocortical carcinoma. In some embodiments, the cancer is pancreatic cancer, or pancreatic ductal carcinoma. In some embodiments, the cancer is pancreatic adenocarcinoma. In some embodiments, the cancer is glioma. In some embodiments, the cancer is malignant peripheral nerve sheath tumors (MPNST). In some embodiments, the cancer is neurofibromatosis-1 associated MPNST. In some embodiments, the cancer is Waldenstrom's macroglobulinemia. In some embodiments, the cancer is medulloblastoma.

In some embodiments, the present invention provides a method of treating a cancer selected from leukemias; Waldenstrom's macroglobulinemia; multiple myeloma; heavy chain disease; and solid tumors, including sarcomas and carcinomas, including fibrosarcoma, myxosarcoma, liposarcoma, chondrosarcoma, osteogenic sarcoma, osteosarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, renal cell carcinoma, colon carcinoma, colorectal carcinoma, pancreatic cancer, breast cancer, ovarian cancer, ovarian epithelial cancer, ovarian carcinoma, fallopian tube cancer, papillary serous cystadenocarcinoma, uterine papillary serous carcinoma (UPSC), hepatocholangiocarcinoma, soft tissue and bone synovial sarcoma, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, hepatocellular carcinoma (HCC), hepatoblastoma, hepatoma, bile duct carcinoma, choriocarcinoma, seminoma, embryonal carcinoma, Wilm's tumor, cervical cancer, uterine cancer, testicular cancer, lung carcinoma, small cell lung carcinoma, bladder carcinoma, epithelial carcinoma, glioma, astrocytoma, glioblastoma multiforme (GBM, also known as glioblastoma), medulloblastoma, craniopharyngioma, ependymoma, pinealoma, hemangioblastoma, acoustic neuroma, oligodendroglioma, schwannoma, neurofibrosarcoma, meningioma, neuroblastoma, and retinoblastoma, comprising administering to a patient in need thereof an effective amount of a disclosed compound or a pharmaceutically acceptable salt thereof.

The present invention further features methods and compositions for the diagnosis, prognosis and treatment of viral-associated cancers, including human immunodeficiency virus (HIV) associated solid tumors, human papilloma virus (HPV)-16 positive incurable solid tumors, and adult T-cell leukemia, which is caused by human T-cell leukemia virus type I (HTLV-I) and is a highly aggressive form of CD4+ T-cell leukemia characterized by clonal integration of HTLV-I in leukemic cells (See clinicaltrials.gov/ct2/show/study/NCT02631746); as well as virus-associated tumors in gastric cancer, nasopharyngeal carcinoma, cervical cancer, vaginal cancer, vulvar cancer, squamous cell carcinoma of the head and neck, and Merkel cell carcinoma. (See clinicaltrials.gov/ct2/show/study/NCT02488759; see also clinicaltrials.gov/ct2/show/study/NCT0240886; clinicaltrials.gov/ct2/show/NCT02426892).

In some embodiments, the present invention provides a method for treating a tumor in a patient in need thereof, comprising administering to the patient any of the compounds, salts or pharmaceutical compositions described herein. In some embodiments, the tumor comprises any of the cancers described herein. In some embodiments, the tumor comprises melanoma cancer. In some embodiments, the tumor comprises breast cancer. In some embodiments, the tumor comprises lung cancer. In some embodiments, the tumor comprises small cell lung cancer (SCLC). In some embodiments, the tumor comprises non-small cell lung cancer (NSCLC).

In some embodiments, the patient is an adult human. In some embodiments, the patient is a child or pediatric patient.

In some embodiments, the tumor is treated by arresting further growth of the tumor. In some embodiments, the tumor is treated by reducing the size (e.g., volume or mass) of the tumor by at least 5%, 10%, 25%, 50%, 75%, 90% or 99% relative to the size of the tumor prior to treatment. In some embodiments, tumors are treated by reducing the quantity of the tumors in the patient by at least 5%, 10%, 25%, 50%, 75%, 90% or 99% relative to the quantity of tumors prior to treatment.

In some embodiments, the present invention provides a method for treating one or more disorders, diseases, and/or conditions wherein the disorder, disease, or condition includes, but is not limited to, a primary immunodeficiency disease or disorder, comprising administering to a patient in need thereof an effective amount of a disclosed compound or pharmaceutically acceptable salt thereof. In some embodiments, the method treats, e.g., ameliorates, a symptom of a primary immunodeficiency, such as neutropenia. Primary immune deficiencies treatable by the methods of the present invention may be present at birth (i.e., congenital), acquired after birth, idiotypic and/or cyclic, and include: warts, hypogammaglobulinemia, infections, myelokathexis (WHIM) syndrome; severe congenital neutropenia (SCN), such as those arising from G6PC3 deficiency (McDermott et al. (2010) Blood 116:2793-2802); GATA2 deficiency (Mono MAC syndrome) (Maciejweski-Duval et al. (2015) J. Leukoc. Biol. 5MA0815-288R (Epub. ahead of printing); idiopathic CD4+ T lymphocytopenia (ICL); and Wiskott-Aldrich Syndrome (WAS). In some embodiments, the present invention provides a method for treating a primary immune deficiency, such as neutropenia, chronic idiopathic neutropenia (CIN), severe CIN, cyclic neutropenia, G6PC3 Deficiency, or Glycogen Storage Disease Ib, comprising administering to a patient in need thereof an effective amount of a disclosed compound or a pharmaceutically acceptable salt thereof.

In some embodiments, the disease, disorder, or condition is selected from B-cell acute lymphoblastic leukemia; B-cell lymphoma; breast tumor; carcinoid tumor; central nervous system tumor; chronic lymphocytic leukemia; colorectal tumor; diffuse large B-cell lymphoma; follicle center lymphoma; glioblastoma; graft versus host disease; hairy cell leukemia; hematological neoplasm; lymphoplasmacytic lymphoma; macroglobulinemia; mantle cell lymphoma; marginal zone B-cell lymphoma; metastatic bladder cancer; metastatic head and neck cancer; metastatic non-small cell lung cancer; metastatic pancreatic cancer; metastatic renal cell carcinoma; multiple myeloma; myelodysplastic syndrome; neuroendocrine tumor; non-Hodgkin lymphoma; ovary tumor; primary mediastinal large B-cell lymphoma; renal cell carcinoma; stomach tumor; systemic mastocytosis; T-cell lymphoma; and transitional cell carcinoma.

In some embodiments, the disease, disorder, or condition is selected from autoimmune encephalomyelitis; B-cell acute lymphoblastic leukemia; B-cell lymphoma; breast tumor; carcinoid tumor; central nervous system tumor; chronic lymphocytic leukemia; colorectal tumor; diffuse large B-cell lymphoma; follicle center lymphoma; glioblastoma; graft versus host disease; hairy cell leukemia; hematological neoplasm; lymphoplasmacytic lymphoma; macroglobulinemia; mantle cell lymphoma; marginal zone B-cell lymphoma; metastatic bladder cancer; metastatic head and neck cancer; metastatic non-small cell lung cancer; metastatic pancreatic cancer; metastatic renal cell carcinoma; multiple myeloma; multiple sclerosis; myelodysplastic syndrome; neuroendocrine tumor; non-Hodgkin lymphoma; ovary tumor; primary mediastinal large B-cell lymphoma; renal cell carcinoma; rheumatoid arthritis; Sjogren's syndrome; stomach tumor; systemic lupus erythematosus; systemic mastocytosis; T-cell lymphoma; transitional cell carcinoma; and urticaria.

In some embodiments, the cancer is metastatic. In some embodiments, the cancer is a B-cell cancer. In some embodiments, the cancer is selected from B-cell acute lymphoblastic leukemia; B-cell lymphoma; breast tumor; carcinoid tumor; central nervous system tumor; chronic lymphocytic leukemia; colorectal tumor; diffuse large B-cell lymphoma; follicle center lymphoma; glioblastoma; hairy cell leukemia; hematological neoplasm; lymphoplasmacytic lymphoma; mantle cell lymphoma; marginal zone B-cell lymphoma; metastatic bladder cancer; metastatic head and neck cancer; metastatic non-small cell lung cancer; metastatic pancreas cancer; metastatic renal cell carcinoma; multiple myeloma; myelodysplastic syndrome; neuroendocrine tumor; non-Hodgkin lymphoma; ovary tumor; primary mediastinal large B-cell lymphoma; renal cell carcinoma; stomach tumor; systemic mastocytosis; T-cell lymphoma; and transitional cell carcinoma.

In some embodiments, the present invention provides a method of treating an autoimmune disease, disorder, or condition in a subject in need thereof, comprising administering to the subject an effective amount of a disclosed compound or a pharmaceutically acceptable salt thereof. In some embodiments, the autoimmune disease, disorder, or condition is graft versus host disease or macroglobulinemia. In some embodiments, the autoimmune disease, disorder, or condition is selected from autoimmune encephalomyelitis, graft versus host disease, macroglobulinemia, multiple sclerosis, rheumatoid arthritis, Sjogren's syndrome, systemic lupus erythematosus, and urticaria.

In some embodiments, the disease, disorder, or condition is selected from inflammatory bowel disease, arthritis, systemic lupus erythematosus (SLE), vasculitis, idiopathic thrombocytopenia purpura (ITP), rheumatoid arthritis, psoriatic arthritis, osteoarthritis, Still's disease, juvenile arthritis, myasthenia gravis, Hashimoto's thyroiditis, Ord's thyroiditis, Graves' disease, autoimmune thyroiditis, Sjogren's syndrome, multiple sclerosis, systemic sclerosis, Lyme neuroborreliosis, Guillain-Barre syndrome, acute disseminated encephalomyelitis, Addison's disease, opsoclonus-myoclonus syndrome, ankylosing spondylosis, antiphospholipid antibody syndrome, aplastic anemia, Fanconi Anemia, autoimmune hepatitis, autoimmune gastritis, pernicious anemia, celiac disease, Goodpasture's syndrome, optic neuritis, scleroderma, primary biliary cirrhosis, Reiter's syndrome, Takayasu's arteritis, temporal arteritis, warm autoimmune hemolytic anemia, Wegener's granulomatosis, psoriasis, alopecia universalis, Behcet's disease, chronic fatigue, dysautonomia, membranous glomerulonephropathy, endometriosis, interstitial cystitis, pemphigus vulgaris, bullous pemphigoid, neuromyotonia, scleroderma, vulvodynia, a hyperproliferative disease, rejection of transplanted organs or tissues, Acquired Immunodeficiency Syndrome (AIDS, caused by HIV), type 1 diabetes, graft versus host disease, transplantation, transfusion, anaphylaxis, allergies (e.g., allergies to plant pollens, latex, drugs, foods, insect poisons, animal hair, animal dander, dust mites, or cockroach calyx), type I hypersensitivity, allergic conjunctivitis, allergic rhinitis, and atopic dermatitis, asthma, appendicitis, atopic dermatitis, asthma, allergy, blepharitis, bronchiolitis, bronchitis, bursitis, cervicitis, cholangitis, cholecystitis, chronic graft rejection, colitis, conjunctivitis, Crohn's disease, cystitis, dacryoadenitis, dermatitis, dermatomyositis, encephalitis, endocarditis, endometritis, enteritis, enterocolitis, epicondylitis, epididymitis, fasciitis, fibrositis, gastritis, gastroenteritis, Henoch-Schonlein purpura, hepatitis, hidradenitis suppurativa, immunoglobulin A nephropathy, interstitial lung disease, laryngitis, mastitis, meningitis, myelitis myocarditis, myositis, nephritis, oophoritis, orchitis, osteitis, otitis, pancreatitis, parotitis, pericarditis, peritonitis, pharyngitis, pleuritis, phlebitis, pneumonitis, pneumonia, polymyositis, proctitis, prostatitis, pyelonephritis, rhinitis, salpingitis, sinusitis, stomatitis, synovitis, tendonitis, tonsillitis, ulcerative colitis, uveitis, vaginitis, vasculitis, or vulvitis, B-cell proliferative disorder, e.g., diffuse large B cell lymphoma, follicular lymphoma, chronic lymphocytic lymphoma, chronic lymphocytic leukemia, acute lymphocytic leukemia, B-cell prolymphocytic leukemia, lymphoplasmacytic lymphoma/Waldenstrom's macroglobulinemia, splenic marginal zone lymphoma, multiple myeloma (also known as plasma cell myeloma), non-Hodgkin's lymphoma, Hodgkin's lymphoma, plasmacytoma, extranodal marginal zone B cell lymphoma, nodal marginal zone B cell lymphoma, mantle cell lymphoma, mediastinal (thymic) large B cell lymphoma, intravascular large B cell lymphoma, primary effusion lymphoma, Burkitt lymphoma/leukemia, or lymphomatoid granulomatosis, breast cancer, prostate cancer, or cancer of the mast cells (e.g., astocytoma, mast cell leukemia, mast cell sarcoma, systemic mastocytosis), bone cancer, colorectal cancer, pancreatic cancer, diseases of the bone and joints including, without limitation, seronegative spondyloarthropathies (including ankylosing spondylitis, psoriatic arthritis and Reiter's disease), systemic sclerosis, osteoporosis, bone cancer, bone metastasis, a thromboembolic disorder, (e.g., myocardial infarct, angina pectoris, reocclusion after angioplasty, restenosis after angioplasty, reocclusion after aortocoronary bypass, restenosis after aortocoronary bypass, stroke, transitory ischemia, a peripheral arterial occlusive disorder, pulmonary embolism, deep venous thrombosis), inflammatory pelvic disease, urethritis, skin sunburn, sinusitis, pneumonitis, encephalitis, meningitis, myocarditis, nephritis, osteomyelitis, myositis, hepatitis, gastritis, enteritis, dermatitis, gingivitis, appendicitis, pancreatitis, cholocystitus, agammaglobulinemia, psoriasis, allergy, irritable bowel syndrome, ulcerative colitis, Sjogren's disease, tissue graft rejection, hyperacute rejection of transplanted organs, asthma, allergic rhinitis, chronic obstructive pulmonary disease (COPD), autoimmune polyglandular disease (also known as autoimmune polyglandular syndrome), autoimmune alopecia, pernicious anemia, glomerulonephritis, dermatomyositis, multiple sclerosis, scleroderma, vasculitis, autoimmune hemolytic and thrombocytopenia states, atherosclerosis, Parkinson's disease, Alzheimer's disease, diabetes, septic shock, psoriatic arthritis, juvenile arthritis, osteoarthritis, chronic idiopathic thrombocytopenia purpura, Waldenstrom macroglobulinemia, atopic dermatitis, degenerative joint disease, vitiligo, autoimmune hypopituitarism, Behcet's disease, scleroderma, mycosis fungoides, and acute inflammatory responses (such as acute respiratory distress syndrome and ischemia/reperfusion injury).

In some embodiments, the autoimmune disease, disorder, or condition is selected from Behcet's disease, pemphigus vulgaris, refractory incomplete systemic lupus erythematosus, retroperitoneal fibrosis, idiopathic thrombocytopeniaurpura (ITP), scleroderma (systemic sclerosis or SSc), pemphigus vulgaris, granulomatosis with polyangiitis, immunoglobulin A nephropathy, small vessel vasculitis, retroperitoneal fibrosis, and psoriasis. In some embodiments, the autoimmune disease is systemic lupus erythematosus (SLE) and/or lupus nephritis (LN). In some embodiments, the autoimmune disease is celiac disease. In some embodiments, the autoimmune disease is inflammatory bowel disease (IBD; e.g., Crohn's disease, ulcerative colitis, collagenous colitis, lymphocytic colitis, diversion colitis, Behcet's disease, and indeterminate colitis). In some embodiments, the autoimmune disease is a neurodegenerative disorder. In some embodiments, the neurodegenerative disorder is multiple sclerosis.

In some embodiments, the present invention provides a method of treating or preventing organ transplant rejection, graft-versus-host disease, or implant rejection, comprising administering to a subject in need thereof an effective amount of a disclosed compound or a pharmaceutically acceptable salt thereof. In some embodiments, the organ transplant is selected from a skin, liver, heart, kidney, pancreas, thymus, small intestine, large intestine, uterus, a vascularized composite allograft (VCA) such as face or hand, bone marrow, allogenic blood and marrow transplant (BMT), cornea, and lung transplant. In some embodiments, the organ transplant rejection is acute or chronic transplant rejection.

In some embodiments, the disease, disorder, or condition is selected from retroperitoneal fibrosis, idiopathic thrombocytopeniaurpura (ITP), scleroderma (systemic sclerosis or SSc), pemphigus vulgaris, granulomatosis with polyangiitis, refractory incomplete systemic lupus erythematosus, inflammatory disease, Abdominal cavity inflammation, Peritonitis, Mesenteritis, Perihepatitis, Salpingoperitonitis, Autoinflammatory disease, Cryopyrin associated periodic syndrome, CINCA syndrome, Familial cold autoinflammatory syndrome, Muckle Wells syndrome, Cardiovascular inflammation, Carditis, Endocarditis, Bacterial endocarditis, Infectious endocarditis, Non infectious endocarditis, Thromboendocarditis, Pericarditis, Chylopericarditis, Dressier syndrome, Pleuropericarditis, Vasculitis, Arteritis, Aortitis, Takayasus arteritis, Endarteritis, HIV associated arteritis, Kawasaki disease, Periarteritis, Polyarteritis nodosa, Temporal arteritis, Extracranial temporal arteritis, Intracranial temporal arteritis, Churg-Strauss syndrome, Cutaneous vasculitis, Perivasculitis, Phlebitis, Lymphangiophlebitis, Thrombophlebitis, Mondor disease, Thromboangiitis, Thromboangiitis obliterans, Thrombophlebitis, Dermatitis, Acrodermatitis, Angiodermatitis, Drug eruption, Erythema multiforme, Serum sickness, Stevens Johnson syndrome, Toxic epidermal necrolysis, Intertrigo, Skin allergy, Atopic dermatitis, Contact dermatitis, Eczema, Fibrosis, Cicatrix, Tissue adhesions, Pulmonary fibrosis, Idiopathic pulmonary fibrosis, Renal fibrosis, Gastrointestinal inflammation, Anusitis, Biliary tract inflammation, Hepatocholangitis, Cholecystitis, Esophagitis, Eosinophilic esophagitis, Gastritis, Gastroduodenitis, Gastroenteritis, Hypertrophic gastritis, Hepatitis, Enterohepatitis, Hepatitis virus infection, Hepatitis A virus infection, Hepatitis B virus infection, Hepatitis C virus infection, Hepatitis D virus infection, Hepatitis E virus infection, Hepatitis F virus infection, Hepatitis G virus infection, Hepatocholangitis, Non-viral hepatitis, Alcoholic hepatitis, Autoimmune hepatitis, Perihepatitis, Steatohepatitis, Non-alcoholic steatohepatitis, Inflammatory bowel disease, Colitis, Diverticulitis, Meckel's diverticulitis, Enterocolitis, Acute enterocolitis, Necrotizing enterocolitis, Ileocecitis, Pseudomembranous colitis, Sigmoiditis, Rectosigmoiditis, Enteritis, Enterocolitis, Acute enterocolitis, Necrotizing enterocolitis, Enterohepatitis, Hemorrhagic enteritis, Ileitis, Ileocecitis, Pouchitis, Jejunitis, Mucositis, Balser necrosis, Necrotizing acute pancreatitis, Proctitis, Rectosigmoiditis, Ulcerative proctitis, Genitourinary tract inflammation, Genital tract inflammation, Female genital tract inflammation, Endometriosis, Parametritis, Pelvic inflammatory disease, Salpingitis, Vaginitis, Atrophic vaginitis, Bartholinitis, Vulvovaginitis, Vulvitis, Vulvovaginitis, Male genital tract inflammation, Balanitis, Epididymitis, Epididymo-orchitis, Orchitis, Epididymo-orchitis, Periorchitis, Prostatitis, Urinary tract inflammation, Nephritis, Alport syndrome, Glomerulonephritis, Focal segmental glomerulosclerosis, IgA nephropathy, Membranoproliferative glomerulonephritis, Membranous glomerulonephritis, Wegener granulomatosis, Lupus nephritis, Pyelitis, Pyelocystitis, Pyelonephritis, Granulomatosis, Allergic granulomatosis, Sarcoidosis, Mastitis, Mouth inflammation, Gingivitis, Pericoronitis, Pharyngitis, Rhinopharyngitis, Sialadenitis, Musculoskeletal system inflammation, Arthritis, Chondrocalcinosis, Gout, Infectious arthritis, Osteoarthritis, Periarthritis, Psoriatic arthritis, Reiter syndrome, Rheumatoid arthritis, Adult onset Stills disease, Felty syndrome, Juvenile rheumatoid arthritis, Bursitis, Dactylitis, Myositis, Dermatomyositis, Inclusion body myositis, Hereditary inclusion body myositis, Sporadic inclusion body myositis, Polymyositis, Pyomyositis, Nervous system inflammation, Meningitis, Arachnoiditis, Aseptic meningitis, Infectious meningitis, Bacterial meningitis, Neisseria meningitidis meningitis, Fungal meningitis, Cryptococcus neoformans meningitis, Parasitic meningitis, Viral meningitis, Neoplastic meningitis, Pachymeningitis, Neuritis, Neuromyelitis optica, Poliovirus infection, Postpoliomyelitis syndrome, Ocular and orbital inflammation, Ocular inflammation, Chorioretinitis, Conjunctivitis, Allergic conjunctivitis, Blepharoconjunctivitis, Keratoconjunctivitis, Infectious keratoconjunctivitis, Ophthalmia neonatorum, Trachoma, Uveitis, Intermediate uveitis, Pars planitis, Orbital inflammatory disease, Idiopathic orbital inflammation, Respiratory tract inflammation, Lower respiratory tract inflammation, Bronchitis, Lung inflammation, Asthma, Asthma attack, Exercise induced asthma, Nocturnal asthma, Occupational asthma, Status asthmaticus, Pleurisy, Upper respiratory tract inflammation, Pharyngitis, Rhinopharyngitis, Rhinitis, Allergic rhinitis, Perennial allergic rhinitis, Seasonal allergic rhinitis, Rhinopharyngitis, Sinusitis, Acute sinusitis, Chronic sinusitis, Ethmoiditis, Kartagener syndrome, Pansinusitis, Serositis, Familial mediterranean fever, Systemic inflammatory response syndrome, Immune disorder, Allergy, Delayed hypersensitivity, Contact dermatitis, Hypersensitivity, Immediate hypersensitivity, Food hypersensitivity, Egg hypersensitivity, Milk hypersensitivity, Oral allergy syndrome, Peanut hypersensitivity, Wheat hypersensitivity, Fungal allergy, Immune complex disease, Arthus reaction, Immediate type hypersensitivity, Respiratory tract allergy, Allergic rhinitis, Perennial allergic rhinitis, Seasonal allergic rhinitis, Asthma, Asthma attack, Exercise induced asthma, Nocturnal asthma, Occupational asthma, Status asthmaticus, Skin allergy, Contact dermatitis, eczema, autoimmune disease, antiphospholipid syndrome, Autoimmune hemolytic anemia, aplastic anemia, cold agglutinin disease, autoimmune hepatitis, autoimmune nervous system disease, autoimmune demyelinating nervous system disease, Stiff person syndrome, Lambert-Eaton syndrome, Cutaneous lupus erythematosus, Discoid lupus erythematosus, Evans syndrome, lupus nephritis, multiple sclerosis (MS), myasthenia gravis, paroxysmal nocturnal hemoglobinuria, primary biliary cirrhosis, painful bladder syndrome, psoriasis, Parapsoriasis, Psoriatic arthritis, Temporal arteritis, Extracranial temporal arteritis, Intracranial temporal arteritis, Vitiligo, Non segmental vitiligo, Segmental vitiligo, graft-versus-host disease, Transplant rejection, Bone marrow transplant rejection, Cell transplant rejection, Corneal transplant rejection, Heart transplant rejection, Kidney transplant rejection, Liver transplant rejection, Lung transplant rejection, organ transplant rejection, intestine transplantation, large intestine transplantation, small intestine transplantation, pancreas transplant rejection, islet cell transplant rejection, skin transplant rejection, tissue transplant rejection, immune deficiency, Agammaglobulinemia, Brutons disease, combined immunodeficiency, HIV, acquired immune deficiency syndrome (AIDS), AIDS related complex, Nezelof syndrome, severe combined immunodeficiency syndrome, adenosine deaminase deficiency, common variable immunodeficiency, DiGeorge syndrome, dysgammaglobulinemia, Immunoglobulin A deficiency, Immunoglobulin G deficiency, phagocyte bactericidal disorder, Chediak Higashi syndrome, chronic granulomatous disease, Job syndrome, Wiskott-Aldrich syndrome, immunoadsorption, lymphatic system disease, adenoid disease, adenoid hypertrophy, adenoid tumor, adenoiditis, lymphadenopathy, Kawasaki disease, lymphadenitis, lymphangiophlebitis, lymphangitis, lymphatic system tumor, Castleman's disease, lymphangioma, cystic hygroma, lymphangiomyoma, interstitial cystitis, a neuromyelitis optica spectrum disorder, juvenile neuronal ceroid lipofuscinosis, autoimmune bullous dermatose, nephrotic syndrome (e.g. including idiopathic nephrotic syndrome or minimal change nephropathy), idiopathic membranous nephropathy, congenital urological abnormality, chronic inflammatory demyelinating polyradiculopathy, immune thrombocytopenia, microscopic polyangiitis, MPO-ANCA vasculitis, Takayasu arteritis, hyperkalemia, Bronchiolitis Obliterans, polycystic liver disease, polyomavirus infection, amyotrophic lateral sclerosis (ALS), familial lipoprotein lipase deficiency, Hurler Syndrome, Fanconi Anemia, Glanzmann Thrombasthenia, severe congenital neutropenia, leukocyte adhesion deficiency, Shwachman-Diamond Syndrome, Diamond-Blackfan Anemia, Dyskeratosis-congenita, Chediak-Higashi Syndrome, histiocytosis, DOCK8 deficiency, uremia (e.g., uremia due to renal transplantation), Epidermolysis Bullosa, Amegakaryocytic Thrombocytopenia, Kostmann Syndrome, Lysosomal Storage Disease, Peroxisomal Disorder, mastocytosis, and Henoch-Schoenlein Purpura Nephritis.

In some embodiments, the disease, disorder, or condition is selected from end stage renal disease (ESRD), allogeneic peripheral haematopoietic stem cell transplant, neuroepithelial tumor, multiple myeloma, agnogenic myeloid metaplasia, leukemia, malignant lymphoma, Smith-Magenis Syndrome, a congenital haemoglobinopathy, a sickle cell disorder, a thalassemic disorder such as beta-thalassemia, type 1 diabetes, severe systemic sclerosis, a myelodysplastic syndrome or neoplasm, antibody-mediated rejection, accelerated phase chronic myelogenous leukemia, adult acute lymphoblastic leukemia, adult acute myeloid leukemia with 1 1q23 (MLL) abnormalities, adult acute myeloid leukemia with Del (5q), adult nasal type extranodal NK/T-Cell lymphoma, anaplastic large cell lymphoma, angioimmunoblastic T-cell lymphoma, blastic phase chronic myelogenous leukemia, childhood acute lymphoblastic leukemia, Burkitt lymphoma, chronic myelogenous leukemia, diffuse large cell lymphoma, immunoblastic large cell lymphoma, nasal type extranodal NK/T-cell lymphoma, chronic myelomonocytic leukemia, chronic phase chronic myelogenous leukemia, cutaneous B-cell non-Hodgkin lymphoma, essential thrombocythemia (ET), extranodal marginal zone B-cell lymphoma of mucosa-associated lymphoid tissue, hepatosplenic T-cell lymphoma, intraocular lymphoma, juvenile myelomonocytic leukemia, nodal marginal zone B-cell lymphoma, noncutaneous extranodal lymphoma, peripheral T-Cell Lymphoma (PTCL), polycythemia vera (PV), post-transplant lymphoproliferative disorder, primary myelofibrosis, recurrent adult diffuse large cell lymphoma, recurrent adult diffuse mixed cell lymphoma, recurrent adult diffuse small cleaved cell lymphoma, recurrent adult grade III lymphomatoid granulomatosis, Hodgkin's lymphoma, recurrent adult immunoblastic large cell lymphoma, recurrent adult lymphoblastic lymphoma, recurrent adult T-cell leukemia/lymphoma, recurrent childhood or adult acute myeloid leukemia, recurrent childhood anaplastic large cell lymphoma, recurrent childhood grade III lymphomatoid granulomatosis, recurrent childhood large cell lymphoma, recurrent childhood lymphoblastic lymphoma, recurrent childhood small noncleaved cell lymphoma, recurrent cutaneous T-cell non-Hodgkin lymphoma, recurrent grade 1 follicular lymphoma, recurrent grade 2 follicular lymphoma, recurrent grade 3 follicular lymphoma, mantle cell lymphoma, marginal Zone Lymphoma, mycosis fungoides/Sezary Syndrome, small lymphocytic lymphoma, non-small cell lung cancer, recurrent/refractory childhood Hodgkin lymphoma, refractory anemia with excess blasts, refractory cytopenia with multilineage dysplasia, hairy cell leukemia, refractory multiple myeloma, relapsing chronic myelogenous leukemia, secondary acute myeloid leukemia, small intestine lymphoma, splenic marginal zone lymphoma, T-cell large granular lymphocyte leukemia, testicular lymphoma, Waldenstrom's Macroglobulinemia (WM), acute myeloid leukemia (in remission), aplastic anemia (AA), chronic myelomonocytic leukemia, indolent Non-Hodgkin's Lymphoma, acute myeloid leukemia (AML), Hodgkin's Lymphoma, a myeloproliferative neoplasm, plasma cell myeloma, refractory anemia, refractory anemia with excess blasts, refractory anemia with ring sideroblasts, refractory cytopenia with multilineage dysplasia, refractory cytopenia with multilineage dysplasia and ring sideroblasts, uveitis, renal interstitial fibrosis, interstitial lung disease, chronic kidney disease, cytomegalovirus infection, antibody-mediated rejection, hepatocellular carcinoma, pancreatic cancer, sarcoma, Ewing's tumor, and hypodiploidy.

In some embodiments, the present invention provides a method of treating a disease, disorder, or condition in a subject in need thereof, comprising administering to the subject an effective amount of a disclosed compound or a pharmaceutically acceptable salt thereof, wherein the disease, disorder, or condition is an autoimmune disorder, an inflammatory disorder, a cancer, or transplant rejection.

In some embodiments, the disease, disorder, or condition is celiac disease, refractory celiac disease, enteropathy-associated T-cell lymphoma, cutaneous T-cell lymphoma, a lymphoproliferative disorder of granular lymphocytes, T-cell leukemia, B-cell chronic lymphocytic leukemia, hairy cell leukemia, acute myelogenous leukemia, solid cancer, inflammatory bowel disease, non-alcoholic fatty liver disease, Epstein-Barr viral infection, eosinophilia, transplant rejection, rheumatoid arthritis, sarcoidosis, or multiple sclerosis.

In some embodiments, the disease, disorder, or condition is a cancer. In some embodiments, the cancer is a cancer of the blood or cells of the immune system. In some embodiments, the cancer is a lymphoma or leukemia. In some embodiments, the cancer is selected from enteropathy-associated T-cell lymphoma, cutaneous T-cell lymphoma, a lymphoproliferative disorder of granular lymphocytes, T-cell leukemia, B-cell chronic lymphocytic leukemia, hairy cell leukemia, acute myelogenous leukemia, and a solid cancer, such as a cancer comprising a solid tumor.

In some embodiments, the present invention provides a method of treating a disease, disorder, or condition in a patient in need thereof, comprising administering to the patient an effective amount of a disclosed compound or pharmaceutically acceptable salt thereof, wherein the disease, disorder, or condition is refractory Celiac disease, inflammatory bowel disease (IBD), Epstein-Barr viral infection, or eosinophilia.

In some embodiments, the disease, disorder, or condition is a fatty liver disease. In some embodiments, the fatty liver disease is fatty liver, non-alcoholic fatty liver disease (NAFLD), non-alcoholic steatohepatitis (NASH), or non-alcoholic steatosis.

In some embodiments, the disease, disorder, or condition is transplant rejection, rheumatoid arthritis, sarcoidosis, or multiple sclerosis.

In some embodiments, the disease, disorder, or condition is selected from Epstein-Barr virus, eosinophilia, and sarcoidosis.

In some embodiments, the compound or pharmaceutically acceptable salt thereof is administered in combination with one or more additional immunomodulatory (e.g., immunosuppressive) agents or other co-administered agents such as tacrolimus, everolimus, sirolimus, a steroid such as prednisone, prednisolone, or dexamethasone, cyclophosphamide, azathioprine, methotrexate, or the like. In some embodiments, the one or more additional immunomodulatory (e.g., immunosuppressive) agents or other co-administered agents such as tacrolimus, everolimus, sirolimus, a steroid such as prednisone, prednisolone, or dexamethasone, cyclophosphamide, azathioprine, methotrexate, or the like. In some embodiments, the compound or pharmaceutically acceptable salt thereof is administered in combination with a calcineurin inhibitor (such as ciclosporin or tacrolimus) and/or prednisolone.

In one embodiment, the cancer is a hematological malignancy.

In one embodiment, the cancer is leukemia. In one embodiment, the cancer is acute myeloid leukemia. In one embodiment, the acute myeloid leukemia is B-cell acute myeloid leukemia. In one embodiment, the cancer is acute lymphocytic leukemia. In one embodiment, the cancer is chronic lymphocytic leukemia/small lymphocytic lymphoma.

In one embodiment, the cancer is a B-cell malignancy.

In one embodiment, the cancer is lymphoma. In one embodiment, the cancer is non-Hodgkin's lymphoma. In one embodiment, the cancer is diffuse large B-cell lymphoma (DLBCL). In one embodiment, the cancer is mantle cell lymphoma (MCL). In one embodiment, the cancer is marginal zone lymphoma (MZL). In one embodiment, the marginal zone lymphoma is splenic marginal zone lymphoma (SMZL). In one embodiment, the cancer is indolent follicular cell lymphoma (iFCL). In one embodiment, the cancer is Burkitt lymphoma.

In one embodiment, the cancer is T-cell lymphoma. In one embodiment, the T-cell lymphoma is anaplastic large cell lymphoma (ALCL). In one embodiment, the T-cell lymphoma is Sezary Syndrome.

In one embodiment, the cancer is Hodgkin's lymphoma.

In one embodiment, the cancer is myelodysplastic syndromes.

In one embodiment, the cancer is myeloma. In one embodiment, the cancer is multiple myeloma. In one embodiment, the multiple myeloma is plasma cell leukemia (PCL).

In one embodiment, the multiple myeloma is newly diagnosed multiple myeloma.

In one embodiment, the multiple myeloma is relapsed or refractory. In one embodiment, the multiple myeloma is refractory to lenalidomide. In one embodiment, the multiple myeloma is refractory to pomalidomide. In one embodiment, the multiple myeloma is refractory to pomalidomide when used in combination with a proteasome inhibitor. In one embodiment, the proteasome inhibitor is selected from bortezomib, carfilzomib, and ixazomib.

In one embodiment, the multiple myeloma is refractory to pomalidomide when used in combination with an inflammatory steroid. In one embodiment, the inflammatory steroid is selected from dexamethasone or prednisone. In one embodiment, the multiple myeloma is refractory to pomalidomide when used in combination with a CD38 directed monoclonal antibody.

In one embodiment, provided herein are methods for achieving a complete response, partial response, or stable disease in a patient, comprising administering to a patient having a cancer provided herein a therapeutically effective amount of a compound provided herein in combination with a second active agent provided herein.

In one embodiment, also provided herein are methods for inducing a therapeutic response assessed with the International Uniform Response Criteria for Multiple Myeloma (IURC) (see Durie BGM, Harousseau J-L, Miguel J S, et al. International uniform response criteria for multiple myeloma. Leukemia, 2006; (10) 10: 1-7) of a patient, comprising administering to a patient having multiple myeloma an effective amount of a therapeutically effective amount of a compound provided herein in combination with a second active agent provided herein.

In another embodiment, provided herein are methods for achieving a stringent complete response, complete response, or very good partial response, as determined by the International Uniform Response Criteria for Multiple Myeloma (IURC) in a patient, comprising administering to a patient having multiple myeloma an effective amount of a therapeutically effective amount of a compound provided herein in combination with a second active agent provided herein.

In another embodiment, provided herein are methods for achieving an increase in overall survival, progression-free survival, event-free survival, time to progression, or disease-free survival in a patient, comprising administering to a subject having cancer an effective amount of a disclosed compound or a pharmaceutically acceptable salt thereof.

In one aspect, the present invention provides a method of treating Waldenstrom's macroglobulinemia, comprising administering to a patient in need thereof an effective amount of a disclosed compound, or a pharmaceutically acceptable salt thereof. Waldenstrom's macroglobulinemia (WM) is a distinct B-cell lymphoproliferative disorder characterized by the proliferation of lymphoplasmacytic cells in the bone marrow in other organs, along with elevated serum levels of monoclonal immunoglobulin M (IgM) gammopathy (Owen 2003; Treon 2013). WM is sometimes referred to as a lymphoplasmacytic lymphoma (LPL) with an associated monoclonal IgM paraprotein. In WM, there is a malignant change to the B-cell in the late stages of maturing, and it continues to proliferate into a clone of identical cells, primarily in the bone marrow but also in the lymph nodes and other tissues and organs of the lymphatic system. Under the microscope, WM cells have characteristics of both B-lymphocytes and plasma cells, and they are called lymphoplasmacytic cells. For that reason, WM is classified as a type of non-Hodgkin's lymphoma called lymphoplasmacytic lymphoma (LPL). About 95% of LPL cases are WM; the remaining 5% do not secrete IgM and consequently are not classified as WM. WM is a very rare disease-only about 1,500 patients are diagnosed with it each year in the US. For reference, approximate normal levels of IgM are described, e.g., in Gonzalez-Quintela et al. (2007) Clinical and Experimental Immunology 151:42-50. Normal levels are approximately: 70 mg/190 ml for males; and 80-250 mg/100 ml for females. See also “Range of normal serum immunoglobulin (IgG, IgA and IgM) values in Nigerians,” Oyeyinko et al., Afr J Med Med Sci 1984 September-December; 13 (3-4): 169-76. Mean values of IgM varied from 65 to 132 mg/100 ml in the males and from 96 to 114 mg/100 ml in the females. For men, normal hemoglobin levels are about 13.5 to 17.5 grams per deciliter; for women, 12.0 to 15.5 grams per deciliter. As a result of proliferation in the bone marrow and other sites, the lymphoplasmacytic cells of WM may interfere with normal functioning. In the bone marrow where blood cells are produced, the WM cells “crowd out” the normal blood cells and may lead to a reduction in normal blood counts; in the lymph nodes and other organs, the WM cells may lead to enlargement of these structures and other complications. Somatic mutation in myeloid differentiation primary response 88 (MYD88) is found in over 90% of patients with WM. Mutations in chemokine (C-X-C motif) receptor 4 (CXCR4) are the next most common mutations and found to be present in 43% of patients with WM (Xu 2016). Published pivotal clinical studies conducted with ibrutinib have reported MYD88 and CXCR4 mutation status affected responses to ibrutinib. In WM, three genomic groups have been delineated on the basis of clinical manifestations and survival: 1) MYD88^{L265} CXCR4^{WT} [with WT indicating wild type], 2) MYD88^{L265P}CXCR4^{WHIM} [with WHIM indicating warts, hypogammaglobulinemia, infections, and myelokathexis], and 3) MYD88^{WT}CXCR4^{WT} WHIM-like mutations result in a gain of function in CXCR4, which in turn decreases chemokine (C-X-C motif) 12 (CXCL12) mediated receptor down regulation and ultimately inhibits egress of cells bearing the mutant CXCR4 from sequestered areas in bone marrow and lymph nodes (Lei 2016; Majumdar 2018). These findings affirm an important role for MYD88 and CXCR4 somatic mutations in the pathogenesis of tumors (Treon 2015 [2]). In the ibrutinib published studies in treatment-naïve and previously treated WM patients, the very good partial response (VGPR) and major response (defined as complete response+VGPR+partial response) rates were highest among patients with MYD88^{L265P}CXCR4^{WT} and significantly lower for those with MYD88^{L265P}CXCR4^{WHIM}. The CXCR4^{WHIM} mutations have been associated with more aggressive disease features, such as higher IgM levels and bone marrow involvement.

Other somatic mutations associated with WM, although with substantially lower frequencies of about 3%-10%, have been identified. These include CD79B; HIST1HH1E; MYBPP1A, ARID1A; HIST1HH1B; TP53; and MLL2, as well as patients with more than one mutation, including TP53/CD79B; RAG2/ARID1A; IST1HH1E/IST1HH1B. Jiminez et al. (2015) Blood 126:2971; Poulain et al. (2016) Blood 128:4092; Hunter et al. (2014) Blood 123:1637; each of which is hereby incorporated by reference.

In some embodiments, a disclosed method comprises treatment of a patient having WM that bears a somatic mutation in the CXCR4 receptor, such as one of those described above. Without wishing to be bound by theory, it is believed that because patients with this mutation have a lesser response to ibrutinib, treatment with a disclosed compound or pharmaceutically acceptable salt thereof will provide improved treatment outcomes for such patients.

Furthermore, without wishing to be bound by theory, it is believed that BTK antagonists have a propensity to induce neutropenia in patients. With its leukocyte mobilization properties of a CXCR4 antagonist, dual CXCR4-BTK antagonists are expected to correct for this leukocytopenia-driven safety risk in patients. Additionally, with their CXCR4 antagonizing properties, it is believed that dual CXCR4-BTK antagonists are less susceptible to bone marrow-induced protection and resistance of tumor cells than is seen with other BTK antagonists in tumor cells that become resistant to cancer therapies due to CXCR4 expression and resultant enhanced adhesion to stroma in bone marrow and other tissue compartments.

Chemokines are major regulators of cell trafficking and adhesion. The chemokine CXCL12 (stromal cell-derived factor-la) is normally expressed on hematopoietic cells such as hematopoietic stem cells (HSCs), T cells, B cells, monocytes and macrophages, neutrophils, and eosinophils (Chatterjee 2014; Nagase 2000). CXCL12 has potent chemotactic activity for lymphocytes and myeloid-derived suppressor cells and is important in homing of HSCs to the bone marrow. When CXCL12 activates CXCR4, it enhances and sustains AKT, extracellular signal-regulated kinase, and Bruton's tyrosine kinase (BTK) signaling pathways, as well as increases cell migration, adhesion, growth, and survival of WM cells (Cao 2014). The chemokine CXCL12 binds primarily to CXC receptor 4 (CXCR4; CD184). The binding of CXCL12 to CXCR4 induces intracellular signaling through several divergent pathways initiating signals related to chemotaxis, cell survival and/or proliferation, increase in intracellular calcium, and gene transcription. CXCR4 is expressed on multiple cell types including lymphocytes, HSCs, endothelial and epithelial cells, and cancer cells. The CXCL12/CXCR4 axis is involved in tumor progression, angiogenesis, metastasis, and survival. This pathway is a target for the development of therapeutic agents that can block the CXCL12/CXCR4 interaction or inhibit downstream intracellular signaling.

In WHIM syndrome, the gain-of-function mutation in CXCR4 results in decreased release of leukocytes into the bloodstream. Treatment with a CXCR4 antagonist has been shown to mobilize leukocytes to beneficially impact the characteristic lymphopenia and leukopenia observed in WHIM patients (Liu 2015; Dale 2011).

At least 40 different nonsense and frameshift mutations in the C-terminal domain of CXCR4 have been described in WM (Poulain 2016; Xu 2016; Stone 2004) The nonsense mutations truncate the distal 15- to 20 amino acid region and the frameshift mutations comprise a region of up to 40 amino acids in the C-terminal domain (Hunter 2014). Nonsense and frameshift mutations are almost equally divided among WM patients. The most common CXCR4 mutation in WM is a nonsense mutation of S338X. The presence of CXCR4 somatic mutations can affect disease presentation in WM. Patients with CXCR4 mutations present with a significantly lower rate of adenopathy, and those with CXCR4 nonsense mutations have an increased bone marrow disease burden, serum IgM levels, and/or risk of symptomatic hyperviscosity (Stone 2004; Treon 2014).

Without wishing to be bound by theory, it is believed that, given the important role of the CXCR4^{WHIM} mutation in ibrutinib-resistance in both clinical and preclinical studies, as well as the other anti-tumor activities of a CXCR4 inhibitor, the effective CXCR4 antagonism by compounds of the present disclosure provides a significant benefit in patients with WM.

In a study published by Varettoni, the median treatment-free survival was significantly shorter in asymptomatic WM patients harboring a CXCR4 mutation at diagnosis (median 51 months) than in those with wild type CXCR4 (median not reached). In multivariate analysis, CXCR4 mutation was an independent prognostic factor for progression from asymptomatic to symptomatic WM requiring therapy (Varettoni 2017).

Waldenstrom's macroglobulinemia patients are often treated with rituximab, an anti-CD20 antibody, as monotherapy or in combination with alkylating agents (bendamustine and cyclophosphamide) or nucleoside analogues (fludarabine and cladribine). Other novel therapies include BTK inhibitors (such as ibrutinib, acalabrutinib and zanubrutinib), proteasome inhibitors (bortezomib and carfilzomib), thalidomide, and everolimus (Buske 2013; Dimopoulos 2014; Treon 2015 [2]; Owen 2014; Dimopoulos 2007; Olszewski 2016). Even though these treatments show some activity, they are not curative, and a standard of care has not been established for WM (Dimopoulos 2017). Therefore, new and/or additional therapeutic options are needed for WM and other cancers and proliferative disorders.

Accordingly, in one aspect, the present invention provides a method of treating a cancer, such as those described herein, by administering to a patient in need thereof an effective amount of a disclosed compound or a pharmaceutically acceptable salt thereof. In some embodiments, the CXCR4 inhibitor and BTK inhibitor portions of the compound act synergistically to provide an improved treatment, e.g., by preventing or reducing immune escape and/or angiogenic escape of the cancer. In some embodiments, the patient has previously been administered another anticancer agent, such as an adjuvant therapy or immunotherapy. In some embodiments, the cancer is refractory.

In some embodiments, the disclosed compound or a pharmaceutically acceptable salt thereof is used in combination with an approved cancer therapy such as radiation, a chemotherapeutic, or an immunotherapy or targeted therapeutic. In some embodiments, the approved cancer therapy is chemotherapy, a targeted drug, a biological therapy, plasmapheresis (plasma exchange), stem cell transplant, or radiation therapy.

In one aspect, the present invention provides a method of treating Waldenstrom's macroglobulinemia (WM) in a patient in need thereof, comprising administering to the patient an effective amount a disclosed compound or a pharmaceutically acceptable salt thereof. In some embodiments, the WM is selected from one of the following genomic groups: 1) MYD88L²⁶⁵ CXCR4WT [with WT indicating wild type], 2) MYD88^{L265P}CXCR4^{WHIM} [with WHIM indicating warts, hypogammaglobulinemia, infections, and myelokathexis], and 3) MYD88^{WT}CXCR4^{WT}. In some embodiments, the WM comprises cells of two, or all three, genomic groups.

Additional agents that may be co-administered with the disclosed compound or pharmaceutically acceptable salt thereof are described in WO 2018/237158, the entire contents of which are hereby incorporated by reference.

Ibrutinib (Ibruvica® Pharmacyclics; Abb Vie) is approved for:
Treatment of mantle cell lymphoma in adult patients who have received at least one prior therapy.
Treatment of chronic lymphocytic leukemia (CLL) or small lymphocytic lymphoma (SLL) [both including CLL and SLL with 17p deletion
Treatment of Waldenström's macroglobulinemia. Marginal zone lymphoma (MZL) who require systemic therapy and have received at least one prior anti-CD20-based therapy
Chronic graft versus host disease (cGVHD) after failure of one or more lines of systemic therapy.

Dosage:
MCL and MZL: 560 mg taken orally once daily.
CLL/SLL, WM, and cGVHD: 420 mg taken orally once daily.

Dose should be taken orally with a glass of water.

In the United States, acalabrutinib (Calquence® AstraZeneca Pharmaceuticals) is approved for:
Treatment of mantle cell lymphoma in adult patients who have received at least one prior therapy;
Treatment of chronic lymphocytic leukemia (CLL) or small lymphocytic lymphoma (SLL).

Recommended dose is 100 mg orally approximately every 12 hours; swallow whole with water and with or without food.

In the United States, zanubrutinib (Brukinsa® Beigene, USA) is approved for:
Treatment of mantle cell lymphoma in adult patients who have received at least one prior therapy.

Recommended dose: 160 mg orally twice daily or 320 mg orally once daily; swallow whole with water and with or without food. Reduce dose in patients with severe hepatic impairment.

Accordingly, in some embodiments, the present invention provides a method of treating mantle cell lymphoma, chronic lymphocytic leukemia (CLL), small lymphocytic lymphoma (SLL) (including CLL and SLL with 17p deletion), Waldenström's macroglobulinemia, marginal zone lymphoma (MZL) (including in a subject who requires systemic therapy and has received at least one prior anti-CD20-based therapy), or chronic graft versus host disease (cGVHD) (including in a patient who has failed one or more lines of systemic therapy), comprising administering to a subject in need thereof an effective amount of a disclosed compound or pharmaceutically acceptable salt thereof.

In some embodiments, the method provides at least a 50% percent decrease in IgM levels from baseline. In some embodiments, the method provides at least a 10%, 20%, 25%, 30%, 35%, 40%, 45%, 50%, 60%, 70%, 75%, 80%, 85%, 90%, 95%, or 98% decrease in IgM levels from baseline. In some embodiments, the method provides about a 50% decrease in IgM levels from baseline. In some embodiments, the method provides about a 10%, 20%, 25%, 30%, 35%, 40%, 45%, 50%, 60%, 70%, or 75% decrease in IgM levels from baseline. In some embodiments, the method provides about a 10-20%, 10-25%, 15-30%, 15-35%, 20-40%, 20-45%, 25-50%, 30-60%, 35-70%, 50-60%, 50-75%, 60-90%, 70-90%, 80-90%, 80-95%, 80-98%, 85-98%, 90-98%, or 95-98% decrease in IgM levels from baseline.

In some embodiments, the method reduces IgM and/or Hgb to within 2 times the normal range for a non-diseased adult human (non-WM patient), 1.5 times, 1.25 times, or to within the normal range for a non-diseased adult human.

In some embodiments, the method decreases Hgb to between 2 times the upper limit of normal (ULN) and the lower limit of normal.

In some embodiments, the compound exerts effects that are greater than additive vs. the corresponding CXCR4 and BTK inhibitors when administered separately.

In some embodiments, the method further comprises the step of obtaining a biological sample from the patient and measuring the amount of a disease-related biomarker.

In some embodiments, the biological sample is a blood sample.

In some embodiments, the disease-related biomarker is selected from circulating CD8+ T cells and the ratio of CD8+ T cells:Treg cells.

In some embodiments, the disease-related biomarker is IgM and/or Hgb. In some embodiments, the biomarker is absolute neutrophil count (ANC).

In some embodiments, the method further comprises administering an additional therapeutic agent, wherein the additional therapeutic agent is an immunostimulatory therapeutic compound.

In some embodiments, the immunostimulatory therapeutic compound is selected from elotuzumab, mifamurtide, an agonist or activator of a toll-like receptor, or an activator of RORγt.

In some embodiments, the method further comprises administering to the patient an additional therapeutic agent, such as an immune checkpoint inhibitor.

In some embodiments, the immune checkpoint inhibitor is selected from nivolumab, pembrolizumab, ipilimumab, avelumab, durvalumab, atezolizumab, or pidilizumab.

In some embodiments, the additional therapeutic agents are selected from an indoleamine (2,3)-dioxygenase (IDO) inhibitor, a Poly ADP ribose polymerase (PARP) inhibitor, a histone deacetylase (HDAC) inhibitor, a CDK4/CDK6 inhibitor, or a phosphatidylinositol 3 kinase (PI3K) inhibitor.

In some embodiments, the IDO inhibitor is selected from epacadostat, indoximod, capmanitib, GDC-0919, PF-06840003, BMS:F001287, Phy906/KD108, or an enzyme that breaks down kynurenine.

In some embodiments, the PARP inhibitor is selected from olaparib, rucaparib, or niraparib.

In some embodiments, the HDAC inhibitor is selected from vorinostat, romidepsin, panobinostat, belinostat, entinostat, or chidamide.

In some embodiments, the CDK 4/6 inhibitor is selected from palbociclib, ribociclib, abemaciclib or trilaciclib.

In some embodiments, the additional therapeutic agent is a BH3 mimetic or a BCL-2 inhibitor. In some embodiments, the BCL-2 inhibitor is venetoclax (Venclexta® (Genentech/AbbVie). Venetoclax is also known as ABT-199, and is presently the only BCL-2 inhibitor approved by the FDA. Kapoor et al. (2020) Cell Death Dis. 11:941. In some embodiments, the BCL-2 inhibitor is.BGB-11417 (Beigene), an investigational drug that works similarly to venetoclax. (Hu et al. (2020) Cancer Research 80 (Suppl): Abstr. 3077 “Abstract 3077: Preclinical characterization of BGB-11417, a potent and selective Bcl-2 inhibitor with superior antitumor activities in haematological tumor models.” In some embodiments, the BCL-2 inhibitor is S55746 (ADIR/Servier) (also known as BCL201), Casara et al. (2018) Oncotarget 920075-20088. In some embodiments, the BCL-2 inhibitor is LOXO-338, a novel, orally available BCL-2 inhibitor. Alencar et al. (2021) Blood 138 (supp.1): Abstract 623: “A First-in-Human Phase 1 Study of Oral LOXO-338, a Selective BCL2 Inhibitor, in Patients with Advanced Hematologic Malignancies (Trial in Progress).” In some embodiments, the BCL-2 inhibitor is. S65487/VOB560, a BCL-2 inhibitor that binds to the BH3 hydrophobic groove of BCL-2. Le Tiran et al. (2021) Cancer Research 81 (Suppl) Abstr. 1276: “Abstract 1276: Identification of S65487/VOB560 as a potent and selective intravenous 2^{nd}-generation BCL-2 inhibitor active in wild-type and clinical mutants resistant to Venetoclax.” The disclosures of the above referenced publications are hereby incorporated by reference herein.

In some embodiments, the method further comprises administering to the subject an additional therapeutic agent, such as an immune checkpoint inhibitor. In some embodiments, the additional therapeutic agent is selected from an indoleamine (2,3)-dioxygenase (IDO) inhibitor, a Poly ADP ribose polymerase (PARP) inhibitor, a histone deacetylase (HDAC) inhibitor, a CDK4/CDK6 inhibitor, or a phosphatidylinositol 3 kinase (PI3K) inhibitor, and an immune checkpoint inhibitor. In some embodiments, the immune checkpoint inhibitor is selected from nivolumab, pembrolizumab, ipilimumab, avelumab, durvalumab, atezolizumab, or pidilizumab.

In some embodiments, the PI3K inhibitor is selected from idelalisib, alpelisib, taselisib, pictilisib, copanlisib, duvelisib, PQR309, or TGR1202.

In some embodiments, the method further comprises administering to the patient a platinum-based therapeutic, a taxane, a nucleoside inhibitor, or a therapeutic agent that interferes with normal DNA synthesis, protein synthesis, cell replication, or will otherwise inhibit rapidly proliferating cells.

In some embodiments, the platinum-based therapeutic is selected from cisplatin, carboplatin, oxaliplatin, nedaplatin, picoplatin, or satraplatin.

In some embodiments, the taxane is selected from paclitaxel, docetaxel, albumin-bound paclitaxel, cabazitaxel, or SID530.

In some embodiments, the therapeutic agent that interferes with normal DNA synthesis, protein synthesis, cell replication, or will otherwise interfere with the replication of rapidly proliferating cells is selected from trabectedin, mechlorethamine, vincristine, temozolomide, cytarabine, lomustine, azacitidine, omacetaxine mepesuccinate, asparaginase Erwinia chrysanthemi, eribulin mesylate, capacetrine, bendamustine, ixabepilone, nelarabine, clorafabine, trifluridine, or tipiracil.

In some embodiments, the patient has a solid tumor. Solid tumors generally comprise an abnormal mass of tissue that typically does not include cysts or liquid areas. In some embodiments, the cancer is Waldenstrom's macroglobulinemia.

In some embodiments, the patient has a resectable solid tumor, meaning that the patient's tumor is deemed susceptible to being removed by surgery. In other embodiments, the patient has an unresectable solid tumor, meaning that the patient's tumor has been deemed not susceptible to being removed by surgery, in whole or in part.

In some embodiments, the present invention provides a method for treating refractory cancer in a patient in need thereof comprising administering to a patient in need thereof an effective amount of a disclosed compound or a pharmaceutically acceptable salt thereof.

In certain embodiments, the patient was previously administered a protein kinase inhibitor. In some embodiments, the patient was previously administered a VEGF-R antagonist. In certain embodiments, the patient was previously administered an immune checkpoint inhibitor. In some embodiments, the patient was previously administered an immune checkpoint inhibitor selected from nivolumab (Opdivo®, Bristol-Myers Squibb), pembrolizumab (Keytruda®, Merck), or ipilumumab (Yervoy®, Bristol-Myers Squibb).

### Co-Administered Therapeutic Agents

In certain embodiments, a disclosed compound or a pharmaceutically acceptable salt thereof, is administered in combination with an additional therapeutic agent. In some embodiments, the compound or a pharmaceutically acceptable salt thereof, or another CXCR4 antagonist, is administered in combination with one, two, or three additional therapeutic agents.

In some embodiments, the additional therapeutic agent is a kinase inhibitor or VEGF-R antagonist. Approved VEGF inhibitors and kinase inhibitors useful in the present invention include: bevacizumab (Avastin®, Genentech/Roche) an anti-VEGF monoclonal antibody; ramucirumab (Cyramza®, Eli Lilly), an anti-VEGFR-2 antibody and ziv-aflibercept, also known as VEGF Trap (Zaltrap®; Regeneron/Sanofi). VEGFR inhibitors, such as regorafenib (Stivarga®, Bayer); vandetanib (Caprelsa®, AstraZeneca); axitinib (Inlyta®, Pfizer); and lenvatinib (Lenvima®, Eisai); Raf inhibitors, such as sorafenib (Nexavar®, Bayer AG and Onyx); dabrafenib (Tafinlar®, Novartis); and vemurafenib (Zelboraf®, Genentech/Roche); MEK inhibitors, such as cobimetanib (Cotellic®, Exelexis/Genentech/Roche); trametinib (Mekinist®, Novartis); Bcr-Abl tyrosine kinase inhibitors, such as imatinib (Gleevec®, Novartis); nilotinib (Tasigna®, Novartis); dasatinib (Sprycel®, BristolMyersSquibb); bosutinib (Bosulif®, Pfizer); and ponatinib (Inclusig®, Ariad Pharmaceuticals); Her2 and EGFR inhibitors, such as gefitinib (Iressa®, AstraZeneca); erlotinib (Tarceeva®, Genentech/Roche/Astellas); lapatinib (Tykerb®, Novartis); afatinib (Gilotrif®, Boehringer Ingelheim); osimertinib (targeting activated EGFR, Tagrisso®, AstraZeneca); and brigatinib (Alunbrig®, Ariad Pharmaceuticals); c-Met and VEGFR2 inhibitors, such as cabozanitib (Cometriq®, Exelexis); and multikinase inhibitors, such as sunitinib (Sutent®, Pfizer); pazopanib (Votrient®, Novartis); ALK inhibitors, such as crizotinib (Xalkori®, Pfizer); ceritinib (Zykadia®, Novartis); and alectinib (Alecenza®, Genentech/Roche); Bruton's tyrosine kinase inhibitors, such as ibrutinib (Imbruvica®, Pharmacyclics/Janssen); and Flt3 receptor inhibitors, such as midostaurin (Rydapt®, Novartis).

Other kinase inhibitors and VEGF-R antagonists that are in development and may be used in the present invention include tivozanib (Aveo Pharmaceuticals); vatalanib (Bayer/Novartis); lucitanib (Clovis Oncology); dovitinib (TKI258, Novartis); Chiauanib (Chipscreen Biosciences); CEP-11981 (Cephalon); linifanib (Abbott Laboratories); neratinib (HKI-272, Puma Biotechnology); radotinib (Supect®, IY5511, Il-Yang Pharmaceuticals, S. Korea); ruxolitinib (Jakafi®, Incyte Corporation); PTC299 (PTC Therapeutics); CP-547,632 (Pfizer); foretinib (Exelexis, GlaxoSmithKline); quizartinib (Daiichi Sankyo) and motesanib (Amgen/Takeda).

In some embodiments, the present invention provides a method of treating Waldenström's macroglobulinemia in a patient in need thereof, comprising administering to the patient an effective amount of a disclosed compound or pharmaceutically acceptable salt thereof in combination with one or more standard of care treatments, or a combination thereof, for Waldenström's macroglobulinemia.

Standard of care treatments for Waldenström's macroglobulinemia are well known to one of ordinary skill in the art and include chemotherapy, or immunotherapy, or a combination thereof. In some embodiments, the standard of care chemotherapy is selected from chlorambucil, cladribine, cyclophosphamide, fludarabine, bendamustine, or a BTK inhibitor, such as ibrutinib, acalabrutinib, or zanubrutinib. In some embodiments, the additional therapeutic agent is ibrutinib (Imbruvica®; Pharmacyclics/Janssen/Abb Vie).

In some embodiments, the present invention provides a method of treating a cancer in a subject in need thereof, comprising administering to the subject an effective amount of a disclosed compound or a pharmaceutically acceptable salt thereof.

### Pharmaceutical Compositions and Formulations

As used herein, the term “pharmaceutically acceptable salt” refers to those salts which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts are well known in the art. For example, S. M. Berge et al., describe pharmaceutically acceptable salts in detail in J. Pharmaceutical Sciences, 1977, 66, 1-19, incorporated herein by reference. Pharmaceutically acceptable salts of the compounds of this invention include those derived from suitable inorganic and organic acids and bases. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other methods used in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, pivalate, propionate, stearate, succinate, sulfate, tartrate, thiocyanate, p-toluenesulfonate, undecanoate, valerate salts, and the like.

Salts derived from appropriate bases include alkali metal, alkaline earth metal, ammonium and N⁺(C₁₋₄alkyl)₄ salts. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, loweralkyl sulfonate and aryl sulfonate.

The term “pharmaceutically acceptable carrier, adjuvant, or vehicle” refers to a non-toxic carrier, adjuvant, or vehicle that does not destroy the pharmacological activity of the compound with which it is formulated. Pharmaceutically acceptable carriers, adjuvants or vehicles that may be used in the compositions of this invention include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat.

A “pharmaceutically acceptable derivative” means any non-toxic salt, ester, salt of an ester or other derivative of a compound of this invention that, upon administration to a patient, is capable of providing, either directly or indirectly, a compound of this invention.

Compositions of the present invention may be administered orally, parenterally, by inhalation spray, topically (as by powders, ointments, or drops), rectally, nasally, buccally, intravaginally, intracisternally, or via an implanted reservoir. The term “parenteral” as used herein includes subcutaneous, intravenous, intramuscular, intra-articular, intra-synovial, intrasternal, intrathecal, intrahepatic, intralesional, and intracranial injection or infusion techniques. Preferably, the compositions are administered orally, intraperitoneally or intravenously. Sterile injectable forms of the compositions of this invention may be aqueous or oleaginous suspension. These suspensions may be formulated according to techniques known in the art using suitable dispersing or wetting agents and suspending agents. The sterile injectable preparation may also be a sterile injectable solution or suspension in a non-toxic parenterally acceptable diluent or solvent, for example as a solution in 1,3-butanediol. Among the acceptable vehicles and solvents that may be employed are water, Ringer's solution and isotonic sodium chloride solution. In addition, sterile, fixed oils are conventionally employed as a solvent or suspending medium.

For this purpose, any bland fixed oil may be employed including synthetic mono- or di-glycerides. Fatty acids, such as oleic acid and its glyceride derivatives are useful in the preparation of injectables, as are natural pharmaceutically acceptable oils, such as olive oil or castor oil, especially in their polyoxyethylated versions. These oil solutions or suspensions may also contain a long-chain alcohol diluent or dispersant, such as carboxymethyl cellulose or similar dispersing agents that are commonly used in the formulation of pharmaceutically acceptable dosage forms including emulsions and suspensions. Other commonly used surfactants, such as Tweens, Spans and other emulsifying agents or bioavailability enhancers which are commonly used in the manufacture of pharmaceutically acceptable solid, liquid, or other dosage forms may also be used for the purposes of formulation.

Pharmaceutically acceptable compositions of this invention may be orally administered in any orally acceptable dosage form including, but not limited to, capsules, tablets, aqueous suspensions or solutions. In the case of tablets for oral use, carriers commonly used include lactose and corn starch. Lubricating agents, such as magnesium stearate, are also typically added. For oral administration in a capsule form, useful diluents include lactose and dried cornstarch. When aqueous suspensions are required for oral use, the active ingredient is combined with emulsifying and suspending agents. If desired, certain sweetening, flavoring or coloring agents may also be added.

Alternatively, pharmaceutically acceptable compositions of this invention may be administered in the form of suppositories for rectal administration. These can be prepared by mixing the agent with a suitable non-irritating excipient that is solid at room temperature but liquid at rectal temperature and therefore will melt in the rectum to release the drug. Such materials include cocoa butter, beeswax and polyethylene glycols.

Pharmaceutically acceptable compositions of this invention may also be administered topically, especially when the target of treatment includes areas or organs readily accessible by topical application, including diseases of the eye, the skin, or the lower intestinal tract. Suitable topical formulations are readily prepared for each of these areas or organs.

Topical application for the lower intestinal tract can be effected in a rectal suppository formulation (see above) or in a suitable enema formulation. Topically-transdermal patches may also be used.

For topical applications, provided pharmaceutically acceptable compositions may be formulated in a suitable ointment containing the active component suspended or dissolved in one or more carriers. Carriers for topical administration of compounds of this invention include, but are not limited to, mineral oil, liquid petrolatum, white petrolatum, propylene glycol, polyoxyethylene, polyoxypropylene compound, emulsifying wax and water. Alternatively, provided pharmaceutically acceptable compositions can be formulated in a suitable lotion or cream containing the active components suspended or dissolved in one or more pharmaceutically acceptable carriers. Suitable carriers include, but are not limited to, mineral oil, sorbitan monostearate, polysorbate 60, cetyl esters wax, cetearyl alcohol, 2-octyldodecanol, benzyl alcohol and water.

For ophthalmic use, provided pharmaceutically acceptable compositions may be formulated as micronized suspensions in isotonic, pH adjusted sterile saline, or, preferably, as solutions in isotonic, pH adjusted sterile saline, either with or without a preservative such as benzylalkonium chloride. Alternatively, for ophthalmic uses, the pharmaceutically acceptable compositions may be formulated in an ointment such as petrolatum.

Pharmaceutically acceptable compositions of this invention may also be administered by nasal aerosol or inhalation. Such compositions are prepared according to techniques well-known in the art of pharmaceutical formulation and may be prepared as solutions in saline, employing benzyl alcohol or other suitable preservatives, absorption promoters to enhance bioavailability, fluorocarbons, and/or other conventional solubilizing or dispersing agents.

Most preferably, pharmaceutically acceptable compositions of this invention are formulated for oral administration. Such formulations may be administered with or without food. In some embodiments, pharmaceutically acceptable compositions of this invention are administered without food. In other embodiments, pharmaceutically acceptable compositions of this invention are administered with food.

The amount of compounds of the present invention that may be combined with the carrier materials to produce a composition in a single dosage form will vary depending upon the host treated and the particular mode of administration. Preferably, provided compositions should be formulated so that a dosage of between 0.01-100 mg/kg body weight/day of the inhibitor can be administered to a patient receiving these compositions.

It should also be understood that a specific dosage and treatment regimen for any particular patient will depend upon a variety of factors, including the activity of the specific compound employed, the age, body weight, general health, sex, diet, time of administration, rate of excretion, drug combination, and the judgment of the treating physician and the severity of the particular disease being treated. The amount of a compound of the present invention in the composition will also depend upon the particular compound in the composition.

The compounds and compositions, according to the method of the present invention, may be administered using any amount and any route of administration effective for treating a cancer, such as those disclosed herein. The exact amount required will vary from subject to subject, depending on the species, age, and general condition of the subject, the severity of the cancer, the particular agent, its mode of administration, and the like. Compounds of the invention are preferably formulated in dosage unit form for ease of administration and uniformity of dosage. The expression “dosage unit form” as used herein refers to a physically discrete unit of agent appropriate for the patient to be treated. It will be understood, however, that the total daily usage of the compounds and compositions of the present invention will be decided by the attending physician within the scope of sound medical judgment. The specific effective dose level for any particular patient or organism will depend upon a variety of factors including the cancer being treated and the severity of the cancer; the activity of the specific compound employed; the specific composition employed; the age, body weight, general health, sex and diet of the patient; the time of administration, route of administration, and rate of excretion of the specific compound employed; the duration of the treatment; drugs used in combination or coincidental with the specific compound employed, and like factors well known in the medical arts.

In certain embodiments, the compounds of the invention may be administered orally or parenterally at dosage levels of about 0.01 mg/kg to about 50 mg/kg and preferably from about 1 mg/kg to about 25 mg/kg, of subject body weight per day, one or more times a day, to obtain the desired therapeutic effect.

Liquid dosage forms for oral administration include, but are not limited to, pharmaceutically acceptable emulsions, microemulsions, solutions, suspensions, syrups and elixirs. In addition to the active compounds, the liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, and perfuming agents.

Injectable preparations, for example, sterile injectable aqueous or oleaginous suspensions may be formulated according to the known art using suitable dispersing or wetting agents and suspending agents. The sterile injectable preparation may also be a sterile injectable solution, suspension or emulsion in a nontoxic parenterally acceptable diluent or solvent, for example, as a solution in 1,3-butanediol. Among the acceptable vehicles and solvents that may be employed are water, Ringer's solution, U.S.P. and isotonic sodium chloride solution. In addition, sterile, fixed oils are conventionally employed as a solvent or suspending medium. For this purpose any bland fixed oil can be employed including synthetic mono- or diglycerides. In addition, fatty acids such as oleic acid are used in the preparation of injectables.

Injectable formulations can be sterilized, for example, by filtration through a bacterial-retaining filter, or by incorporating sterilizing agents in the form of sterile solid compositions which can be dissolved or dispersed in sterile water or other sterile injectable medium prior to use.

In order to prolong the effect of a compound of the present invention, it is often desirable to slow the absorption of the compound from subcutaneous or intramuscular injection. This may be accomplished by the use of a liquid suspension of crystalline or amorphous material with poor water solubility. The rate of absorption of the compound then depends upon its rate of dissolution that, in turn, may depend upon crystal size and crystalline form. Alternatively, delayed absorption of a parenterally administered compound form is accomplished by dissolving or suspending the compound in an oil vehicle. Injectable depot forms are made by forming microencapsule matrices of the compound in biodegradable polymers such as polylactide-polyglycolide. Depending upon the ratio of compound to polymer and the nature of the particular polymer employed, the rate of compound release can be controlled. Examples of other biodegradable polymers include poly(orthoesters) and poly(anhydrides). Depot injectable formulations are also prepared by entrapping the compound in liposomes or microemulsions that are compatible with body tissues.

Compositions for rectal or vaginal administration are preferably suppositories which can be prepared by mixing the compounds of this invention with suitable non-irritating excipients or carriers such as cocoa butter, polyethylene glycol or a suppository wax which are solid at ambient temperature but liquid at body temperature and therefore melt in the rectum or vaginal cavity and release the active compound.

Solid dosage forms for oral administration include capsules, tablets, pills, powders, and granules. In such solid dosage forms, the active compound is mixed with at least one inert, pharmaceutically acceptable excipient or carrier such as sodium citrate or dicalcium phosphate and/or a) fillers or extenders such as starches, lactose, sucrose, glucose, mannitol, and silicic acid, b) binders such as, for example, carboxymethylcellulose, alginates, gelatin, polyvinylpyrrolidinone, sucrose, and acacia, c) humectants such as glycerol, d) disintegrating agents such as agar-agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. In the case of capsules, tablets and pills, the dosage form may also comprise buffering agents.

Solid compositions of a similar type may also be employed as fillers in soft and hard-filled gelatin capsules using such excipients as lactose or milk sugar as well as high molecular weight polyethylene glycols and the like. The solid dosage forms of tablets, dragees, capsules, pills, and granules can be prepared with coatings and shells such as enteric coatings and other coatings well known in the pharmaceutical formulating art. They may optionally contain opacifying agents and can also be of a composition that they release the active ingredient(s) only, or preferentially, in a certain part of the intestinal tract, optionally, in a delayed manner. Examples of embedding compositions that can be used include polymeric substances and waxes. Solid compositions of a similar type may also be employed as fillers in soft and hard-filled gelatin capsules using such excipients as lactose or milk sugar as well as high molecular weight polethylene glycols and the like.

The active compounds can also be in micro-encapsulated form with one or more excipients as noted above. The solid dosage forms of tablets, dragees, capsules, pills, and granules can be prepared with coatings and shells such as enteric coatings, release controlling coatings and other coatings well known in the pharmaceutical formulating art. In such solid dosage forms the active compound may be admixed with at least one inert diluent such as sucrose, lactose or starch. Such dosage forms may also comprise, as is normal practice, additional substances other than inert diluents, e.g., tableting lubricants and other tableting aids such a magnesium stearate and microcrystalline cellulose. In the case of capsules, tablets and pills, the dosage forms may also comprise buffering agents. They may optionally contain opacifying agents and can also be of a composition that they release the active ingredient(s) only, or preferentially, in a certain part of the intestinal tract, optionally, in a delayed manner. Examples of embedding compositions that can be used include polymeric substances and waxes.

Dosage forms for topical or transdermal administration of a compound of this invention include ointments, pastes, creams, lotions, gels, powders, solutions, sprays, inhalants or patches. The active component is admixed under sterile conditions with a pharmaceutically acceptable carrier and any needed preservatives or buffers as may be required. Ophthalmic formulation, ear drops, and eye drops are also contemplated as being within the scope of this invention. Additionally, the present invention contemplates the use of transdermal patches, which have the added advantage of providing controlled delivery of a compound to the body. Such dosage forms can be made by dissolving or dispensing the compound in the proper medium. Absorption enhancers can also be used to increase the flux of the compound across the skin. The rate can be controlled by either providing a rate controlling membrane or by dispersing the compound in a polymer matrix or gel.

Inasmuch as it may be desirable to administer a combination of active compounds, for example, for the purpose of treating a particular disease or condition, it is within the scope of the present invention that two or more pharmaceutical compositions, at least one of which contains a compound in accordance with the invention, may conveniently be combined in the form of a kit suitable for co-administration of the compositions. Thus the kit of the invention includes two or more separate pharmaceutical compositions, at least one of which contains a compound of the invention, and means for separately retaining said compositions, such as a container, divided bottle, or divided foil packet. An example of such a kit is the familiar blister pack used for the packaging of tablets, capsules and the like.

The kit of the invention is particularly suitable for administering different dosage forms, for example, oral and parenteral, for administering the separate compositions at different dosage intervals, or for titrating the separate compositions against one another. To assist compliance, the kit typically includes directions for administration and may be provided with a memory aid.

The examples below explain the invention in more detail. The following preparations and examples are given to enable those skilled in the art to more clearly understand and to practice the present invention. The present invention, however, is not limited in scope by the exemplified embodiments, which are intended as illustrations of single aspects of the invention only, and methods which are functionally equivalent are within the scope of the invention. Indeed, various modifications of the invention in addition to those described herein will become apparent to those skilled in the art from the foregoing description and accompanying drawings. Such modifications are intended to fall within the scope of the appended claims.

The contents of each document cited in the specification are herein incorporated by reference in their entireties.

### EXEMPLIFICATION

The following examples are intended to illustrate the invention and are not to be construed as being limitations thereon. Unless otherwise stated, one or more tautomeric forms of compounds of the examples described hereinafter may be prepared in situ and/or isolated. All tautomeric forms of compounds of the examples described hereafter should be considered to be disclosed. Temperatures are given in degrees centigrade. If not mentioned otherwise, all evaporations were carried out in vacuo with a rotary evaporator. Analytical samples were dried in vacuo (1-5 mmHg) at rt. Thin layer chromatography (TLC) was performed on silica gel plates, spots were visualized by UV light (214 and 254 nm). Purification by column and flash chromatography was carried out using silica gel (200-300 mesh). Solvent systems are reported as mixtures by volume. All NMR spectra were recorded on a Bruker 400 (400 MHz) spectrometer. 1H chemical shifts are reported in 8 values in ppm with the deuterated solvent as the internal standard. Data are reported as follows: chemical shift, multiplicity (s=singlet, d=doublet, t=triplet, q=quartet, br=broad, m=multiplet), coupling constant (Hz), integration. LCMS spectra were obtained on an Agilent 1200 series 6110 or 6120 mass spectrometer with electrospray ionization and excepted as otherwise indicated, the general LCMS condition was as follows: Waters X Bridge C18 column (50 mm*4.6 mm*3.5 um), Flow Rate: 2.0 mL/min, the column temperature: 40° C.

All starting materials, building blocks, reagents, acids, bases, dehydrating agents, solvents, and catalysts utilized to synthesis the compounds of the present invention are either commercially available or can be produced by organic synthesis methods known to one of ordinary skill in the art (Houben-Weyl 4th Ed. 1952, Methods of Organic Synthesis, Thieme, Volume 21). Further, the compounds of the present invention can be produced by organic synthesis methods known to one of ordinary skill in the art as shown in the following examples.

As depicted in the Examples below, in certain exemplary embodiments, compounds are prepared according to the following general procedures. It will be appreciated that, although the general methods depict the synthesis of certain compounds of the present invention, the following general methods, and other methods known to one of ordinary skill in the art, can be applied to all compounds and subclasses and species of each of these compounds, as described herein.

### Example 1: Synthesis of Compound I-1

### Synthetic Scheme of Compound I-1

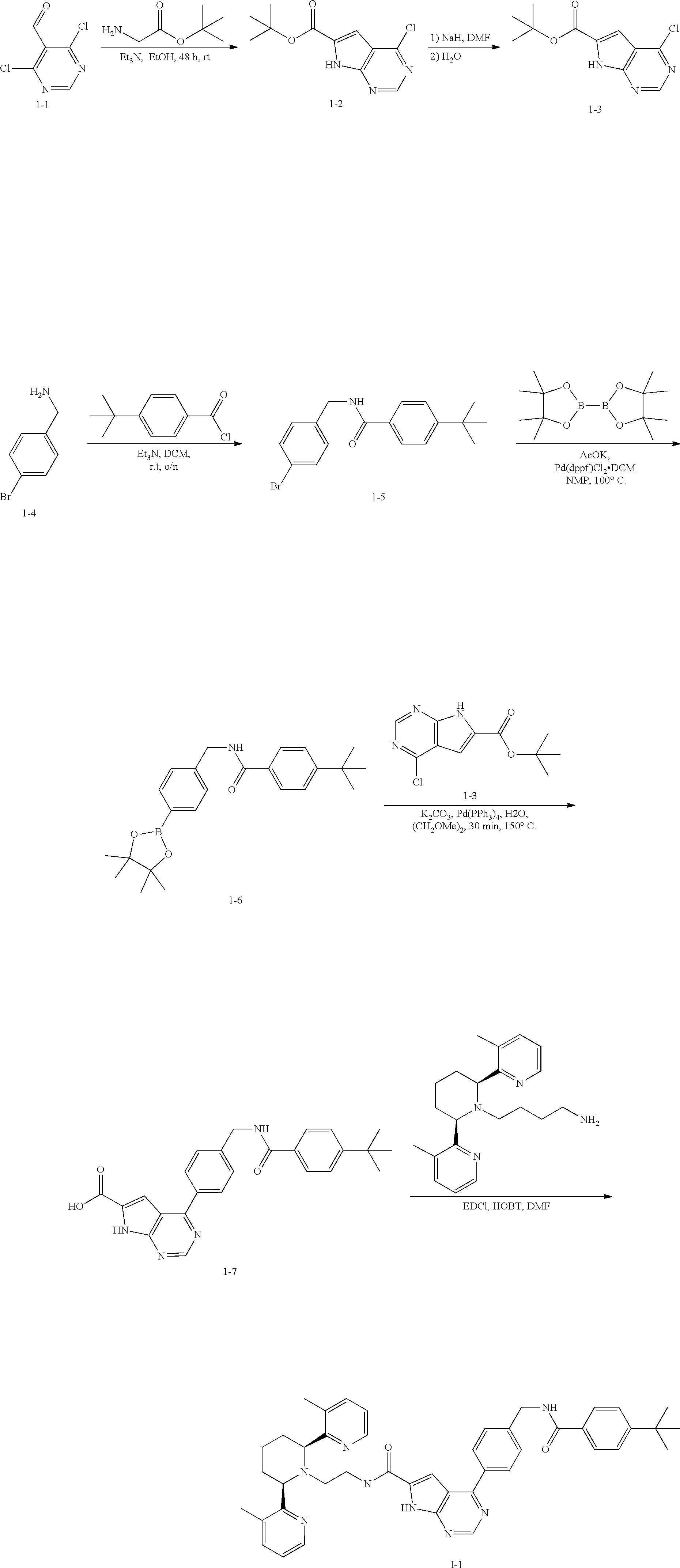

### 1.1 the Synthesis of Intermediate 1-2

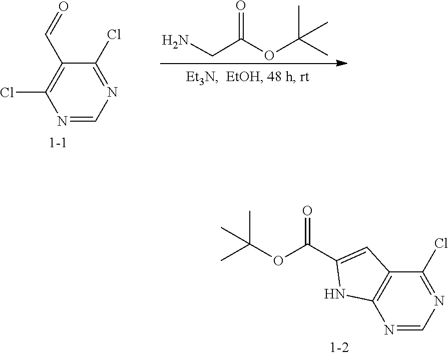

To a solution of 4,6-dichloropyrimidine-5-carbaldehyde (1-1, 30.0 g, 170.5 mmol) in dry EtOH (16 mL) was slowly added tert-butyl 2-aminoacetate (22.3 g, 170.5 mmol) followed by triethylamine (43.1 g, 426.3 mmol) and stirred at room temperature for 48 h. The solvent was removed under reduced pressure and the crude was diluted with dichloromethane and washed with water, and extracted with dichloromethane (500 mL×3). The combined organic layers were washed with brine, dried over anhydrous Na₂SO₄, filtered and concentrated in vacuo to give the residue, which was purified by column chromatography to give tert-butyl 4-chloro-5-hydroxy-6,7-dihydro-5H-pyrrolo[2,3-d]pyrimidine-6-carboxylate (1-2, 5.2 g, 11%) as light-yellow solid.

LCMS (Agilent LCMS 1200-6120, Column: Waters X-Bridge C18 (50 mm*4.6 mm*3.5 μm); Column Temperature: 40° C.; Flow Rate: 2.0 mL/min; Mobile Phase: from 95% [water+10 mM NH₄HCO₃] and 5% [CH₃CN] to 0% [water+10 mM NH₄HCO₃] and 100% [CH₃CN] in 1.6 min, then under this condition for 1.4 min, finally changed to 95% [water+10 mM NH₄HCO₃] and 5% [CH₃CN] in 0.1 min and under this condition for 0.7 min.) Purity: 95.68%. Rt=1.362 min and 1.414 min; MS Calcd.: 271.1; MS Found: 272.0 [M+H]⁺.

### 1.2 the Synthesis of 1-3

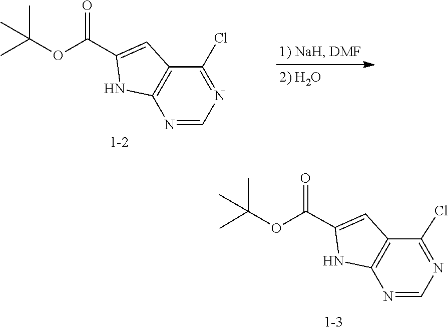 [SMILES: CC(C)(C)OC(=O)c1cc2c(Cl)ncnc2[nH]1 ; CC(C)(C)OC(=O)c1cc2c(Cl)ncnc2[nH]1]

To a solution of tert-butyl 4-chloro-5-hydroxy-6, 7-dihydro-5H-pyrrolo[2, 3-d]pyrimidine-6-carboxylate (1-2, 2.0 g, 7.38 mmol) in DMF (25 mL) was added sodium hydride (285 mg, 7.38 mmol) at 0° C. and then stirred at room temperature for 1 h. After completion of the reaction indicated by LCMS, the mixture was quenched with water, extracted with dichloromethane (200 mL×3). The combined organic layers were washed with brine, dried over anhydrous Na₂SO₄, filtered and concentrated in vacuo to give the residue, which was purified by column chromatography to give tert-butyl4-chloro-5-hydroxy-6,7-dihydro-5H-pyrrolo[2,3-d]pyrimidine-6-carboxylate (1-3, 1.6 g, 85%) as light yellow solid.

LCMS (Agilent LCMS 1200-6120, Column: Waters X-Bridge C18 (50 mm*4.6 mm*3.5 μm); Column Temperature: 40° C.; Flow Rate: 2.0 mL/min; Mobile Phase: from 95% [water+10 mM NH₄HCO₃] and 5% [CH₃CN] to 0% [water+10 mM NH₄HCO₃] and 100% [CH₃CN] in 1.6 min, then under this condition for 1.4 min, finally changed to 95% [water+10 mM NH₄HCO₃] and 5% [CH₃CN] in 0.1 min and under this condition for 0.7 min.) Purity: 100.00%. Rt=1.840 min; MS Calcd.: 253.1; MS Found: 254.1 [M+H]⁺.

1.3 The synthesis of 1-5

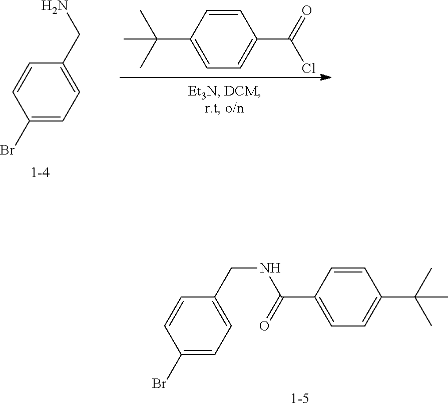

To a solution of (4-bromophenyl) methanamine (1-4, 2.0 g, 10.26 mmol) and 4-tert-butylbenzoyl chloride (2.0 g, 10.26 mmol) in DCM (40 mL) was added triethylamine (2.2 g, 21.5 mmol) at room temperature for overnight. After completion of the reaction indicated by LCMS, the mixture was quenched with water, extracted with dichloromethane (200 mL×3). The combined organic layers were washed with brine, dried over anhydrous Na₂SO₄, filtered and concentrated in vacuo to give the residue, which was purified by column chromatography to give N-(4-bromobenzyl)-4-tert-butylbenzamide (1-5, 3.2 g, 86%) as light yellow solid.

LCMS (Agilent LCMS 1200-6120, Column: Waters X-Bridge C18 (50 mm*4.6 mm*3.5 μm); Column Temperature: 40° C.; Flow Rate: 2.0 mL/min; Mobile Phase: from 95% [water+10 mM NH₄HCO₃] and 5% [CH₃CN] to 0% [water+10 mM NH₄HCO₃] and 100% [CH₃CN] in 1.6 min, then under this condition for 1.4 min, finally changed to 95% [water+10 mM NH₄HCO₃] and 5% [CH₃CN] in 0.1 min and under this condition for 0.7 min.) Purity: 100.00%. Rt=2.063 min; MS Calcd.: 345.1; MS Found: 346.0 [M+H]⁺.

### 1.4 the Synthesis of 1-6

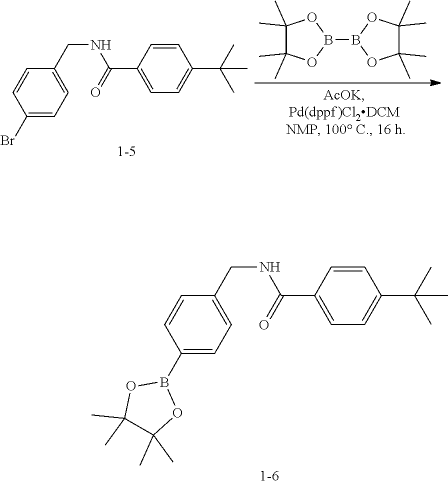

A solution of N-(4-bromobenzyl)-4-tert-butylbenzamide (1-5, 2.0 g, 5.78 mmol), 4,4,4′,4′,5,5,5′,5′-octamethyl-2,2′-bi (1,3,2-dioxaborolane) (1.76 g, 6.94 mmol), AcOK (1.7 g, 17.34 mmol) and Pd(dppf)Cl₂·DCM (490 mg, 0.6 mmol) in NMP (30 mL) under N₂ at room temperature was stirred at 100° C. for 16 hrs. After cooling to room temperature the mixture was diluted with EA and washed with water. The organic phase was dried over Na₂SO₄, filtered and concentrated. The crude material was purified by flash column chromatography on silica gel (PE:EA=10:1) to give4-tert-butyl-N-(4-(4,4,5,5-tetramethyl-1,3,2-dioxaborolan-2-yl)benzyl)benzamide (1-6, 1.8 g, 78% yield) as a white solid.

LCMS (Agilent LCMS 1200-6110, Column: Waters X-Bridge C18 (50 mm*4.6 mm*3.5 μm); Column Temperature: 40° C.; Flow Rate: 1.5 mL/min; Mobile Phase: from 95% [water+0.05% TFA] and 5% [CH₃CN+0.05% TFA] to 0% [water+0.05% TFA] and 100% [CH₃CN+0.05% TFA] in 0.8 min, then under this condition for 0.4 min, finally changed to 95% [water+0.05% TFA] and 5% [CH₃CN+0.05% TFA] and under this condition for 0.01 min). Purity: 53.66%, Rt=1.998 min; MS Calcd.: 393.3; MS Found: 394.2 [M+H]⁺.

### 1.5 the Synthesis of 1-7

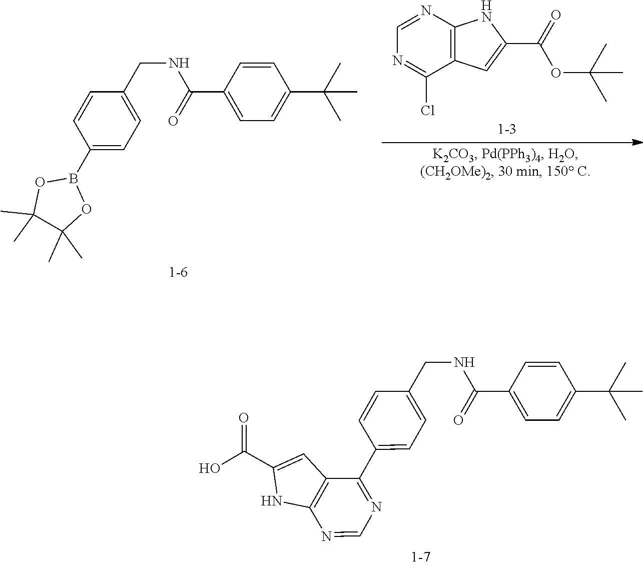 [SMILES: CC(C)(C)c1ccc(C(=O)NCc2ccc(B3OC(C)(C)C(C)(C)O3)cc2)cc1 ; CC(C)(C)OC(=O)c1cc2c(Cl)ncnc2[nH]1 ; CC(C)(C)c1ccc(C(=O)NCc2ccc(-c3ncnc4[nH]c(C(=O)O)cc34)cc2)cc1]

A solution of 4-tert-butyl-N-(4-(4,4,5,5-tetramethyl-1,3,2-dioxaborolan-2-yl)benzyl)benzamide (1-6, 800 mg, 2.04 mmol), tert-butyl 4-chloro-7H-pyrrolo[2,3-d]pyrimidine-6-carboxylate (1-3, 516 mg, 2.04 mmol), K₂CO₃ (845 mg, 6.12 mmol), Pd(PPh₃)₄ (116 mg, 0.1 mmol) in DME (10 mL) and water (10 mL) was heated in the microwave at 150° C. for 30 min. After cooling to room temperature the mixture was concentrated to give the residue, the crude material was purified by flash column chromatography to give tert-butyl4-(4-((4-tert-butylbenzamido)methyl)phenyl)-7H-pyrrolo[2,3-d]pyrimidine-6-carboxylate (1-7, 480 mg, 55% yield) as a purple solid.

LCMS (Agilent LCMS 1200-6110, Column: Waters X-Bridge C18 (50 mm*4.6 mm*3.5 μm); Column Temperature: 40° C.; Flow Rate: 1.5 mL/min; Mobile Phase: from 95% [water+0.05% TFA] and 5% [CH₃CN+0.05% TFA] to 0% [water+0.05% TFA] and 100% [CH₃CN+0.05% TFA] in 0.8 min, then under this condition for 0.4 min, finally changed to 95% [water+0.05% TFA] and 5% [CH₃CN+0.05% TFA] and under this condition for 0.01 min). Purity: 95.76%, Rt=1.597 min; MS Calcd.: 428.2; MS Found: 429.0 [M+H]⁺.

### 1.6 the Synthesis of Compound I-1

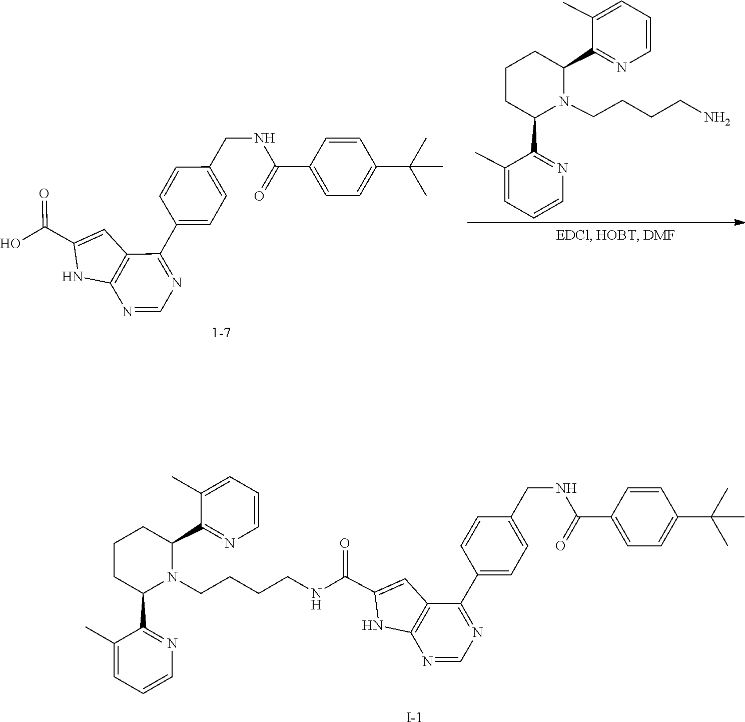

A solution of 4-(4-((4-tert-butylbenzamido)methyl)phenyl)-7H-pyrrolo[2,3-d]pyrimidine-6-carboxylic acid (1-7, 140 mg, 0.33 mmol), 4-((2R,6S)-2,6-bis(3-methylpyridin-2-yl)piperidin-1-yl) butan-1-amine (110 mg, 0.33 mmol), EDCI (95 mg, 0.50 mmol) and DIPEA (128 mg, 0.99 mmol) in DMF (10 mL) at room temperature for 1 hr, followed by adding HOBT (68 mg, 0.50 mmol), than the reaction mixture was allowed to stir at room temperature overnight. After completion of the reaction indicated by LCMS, the mixture was quenched with water and concentrated in vacuum to give the residue, which was purified by column chromatography and prep-HPLC to give I-1 (96.23 mg, 39%) as white solid.

LCMS (Agilent LCMS 1200-6120, Column: Waters X-Bridge C18 (50 mm*4.6 mm*3.5 μm); Column Temperature: 40° C.; Flow Rate: 2.0 mL/min; Mobile Phase: from 95% [water+10 mM NH₄HCO₃] and 5% [CH₃CN] to 0% [water+10 mM NH₄HCO₃] and 100% [CH₃CN] in 1.6 min, then under this condition for 1.4 min, finally changed to 95% [water+10 mM NH₄HCO₃] and 5% [CH₃CN] in 0.1 min and under this condition for 0.7 min.) Purity: 100.00%. Rt=1.986 min; MS Calcd.: 748.4; MS Found: 749.0 [M+H]⁺.

HPLC (Agilent HPLC 1200, Column: L-column2 ODS (150 mm*4.6 mm*5.0 μm); Column Temperature: 40° C.; Flow Rate: 1.0 mL/min; Mobile Phase: from 90% [(total 10 mM AcONH₄) H₂O/MeCN=900/100 (v/v)] and 10% [total 10 mM AcONH₄) H₂O/MeCN=100/900 (v/v)] to 15% [total 10 mM AcONH₄) H₂O/MeCN=900/100 (v/v)] and 85% [total 10 mM AcONH₄) H₂O/MeCN=100/900 (v/v)] in 5 min, then under this condition for 10 min, finally changed to 90% [(total 10 mM AcONH₄) H₂O/MeCN=900/100 (v/v)] and 10% [total 10 mM AcONH₄) H₂O/MeCN=100/900 (v/v)] in 0.1 min and under this condition for 5 min. Purity: 100.00%, Rt=10.314 min; MS Calcd.: 748.4; MS Found: 749.0 [M+H]⁺. ¹H NMR (400 MHZ, DMSO-d₆)δ: 12.63 (s, 1H), 9.12 (t, J=6.0 Hz, 1H), 8.87 (d, J=4.4 Hz, 1H), 8.29 (s, 3H), 8.13 (d, J=8.4 Hz, 2H), 7.88 (d, J=8.4 Hz, 2H), 7.53 (dd, J 1=8.0 Hz, J 2=14.0 Hz, 4H), 7.47 (s, 1H), 7.39 (d, J=5.2 Hz, 2H), 7.02 (s, 2H), 4.60 (d, J=6.0 Hz, 2H), 4.18 (s, 2H), 2.67 (d, J=1.2 Hz, 2H), 2.50 (d, J=1.6 Hz, 6H), 2.25 (s, 2H), 2.15-12.08 (m, 2H), 1.99-1.90 (m, 1H), 1.63-1.56 (m, 1H), 1.47 (d, J=4.4 Hz, 2H), 1.31 (s, 9H), 0.65 (d, J=2.4 Hz, 2H), 0.34-0.25 (m, 1H).

### Example 2: Synthesis of Compound I-2

### 2.1 the Synthetic of Compound I-2

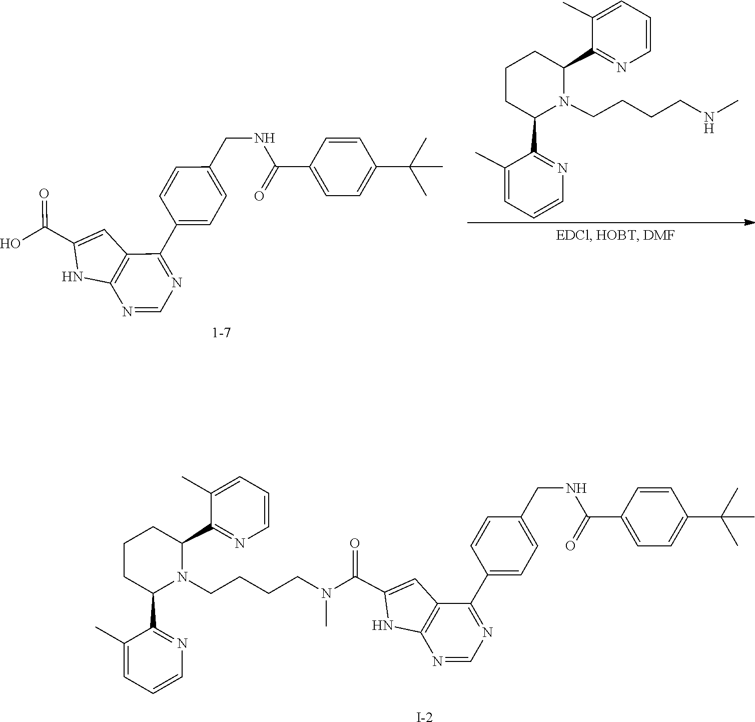

A solution of 4-(4-((4-tert-butylbenzamido)methyl)phenyl)-7H-pyrrolo[2,3-d]pyrimidine-6-carboxylic acid (1-7, 120 mg, 0.28 mmol), 4-((2R,6S)-2,6-bis(3-methylpyridin-2-yl)piperidin-1-yl)-N-methylbutan-1-amine (95 mg, 0.28 mmol), EDCI (81 mg, 0.42 mmol) and DIPEA (108 mg, 0.84 mmol) in DMF (10 mL) at room temperature for 1 hr, followed by adding HOBT (57 mg, 0.42 mmol), than the reaction mixture was allowed to stir at room temperature overnight. After completion of the reaction indicated by LCMS, the mixture was quenched with water and concentrated in vacuum to give the residue, which was purified by column chromatography and prep-HPLC to give I-2 (53.64 mg, 25%) as white solid. LCMS (Agilent LCMS 1200-6120, Column: Waters X-Bridge C18 (50 mm*4.6 mm*3.5 μm); Column Temperature: 40° C.; Flow Rate: 2.0 mL/min; Mobile Phase: from 95% [water+10 mM NH₄HCO₃] and 5% [CH₃CN] to 0% [water+10 mM NH₄HCO₃] and 100% [CH₃CN] in 1.6 min, then under this condition for 1.4 min, finally changed to 95% [water+10 mM NH₄HCO₃] and 5% [CH₃CN] in 0.1 min and under this condition for 0.7 min.) Purity: 95.69%. Rt=2.006 min; MS Calcd.: 762.4; MS Found: 763.0 [M+H]⁺. HPLC (Agilent HPLC 1200, Column: L-column2 ODS (150 mm*4.6 mm*5.0 μm); Column Temperature: 40° C.; Flow Rate: 1.0 mL/min; Mobile Phase: from 90% [(total 10 mM AcONH₄) H₂O/MeCN=900/100 (v/v)] and 10% [total 10 mM AcONH₄) H₂O/MeCN=100/900 (v/v)] to 15% [total 10 mM AcONH₄) H₂O/MeCN=900/100 (v/v)] and 85% [total 10 mM AcONH₄) H₂O/MeCN=100/900 (v/v)] in 5 min, then under this condition for 10 min, finally changed to 90% [(total 10 mM AcONH₄) H₂O/MeCN=900/100 (v/v)] and 10% [total 10 mM AcONH₄) H₂O/MeCN=100/900 (v/v)] in 0.1 min and under this condition for 5 min. Purity: 95.44%, Rt=10.373 min; MS Calcd.: 762.4; MS Found: 763.0 [M+H]⁺. ¹H NMR (400 MHz, DMSO-d₆)δ: 12.38 (s, 1H), 8.91 (d, J=8.4 Hz, 2H), 8.23 (s, 2H), 8.16 (d, J=8.4 Hz, 2H), 8.87 (d, J=8.4 Hz, 2H), 8.13 (d, J=8.4 Hz, 2H), 7.55 (d, J=8.0 Hz, 2H), 7.53 (dd, J 1=2.0 Hz, J 2=6.8 Hz, 3H), 7.32 (s, 2H), 6.99 (s, 2H), 6.90 (s, 1H), 4.61 (d, J=6.0 Hz, 2H), 4.07 (d, J=8.8 Hz, 2H), 2.88 (t, J=6.8 Hz, 2H), 2.82 (s, 3H), 2.43 (s, 6H), 2.20 (s, 2H), 2.05 (d, J=12.0 Hz, 2H), 1.90 (d, J=12.0 Hz, 1H), 1.57 (d, J=12.8 Hz, 1H), 1.47 (d, J=5.6 Hz, 2H), 1.31 (s, 9H), 0.74 (s, 2H), 0.42-0.35 (m, 1H).

### Example 3: Synthesis of Compound I-3

### Synthetic Scheme of Compound I-3

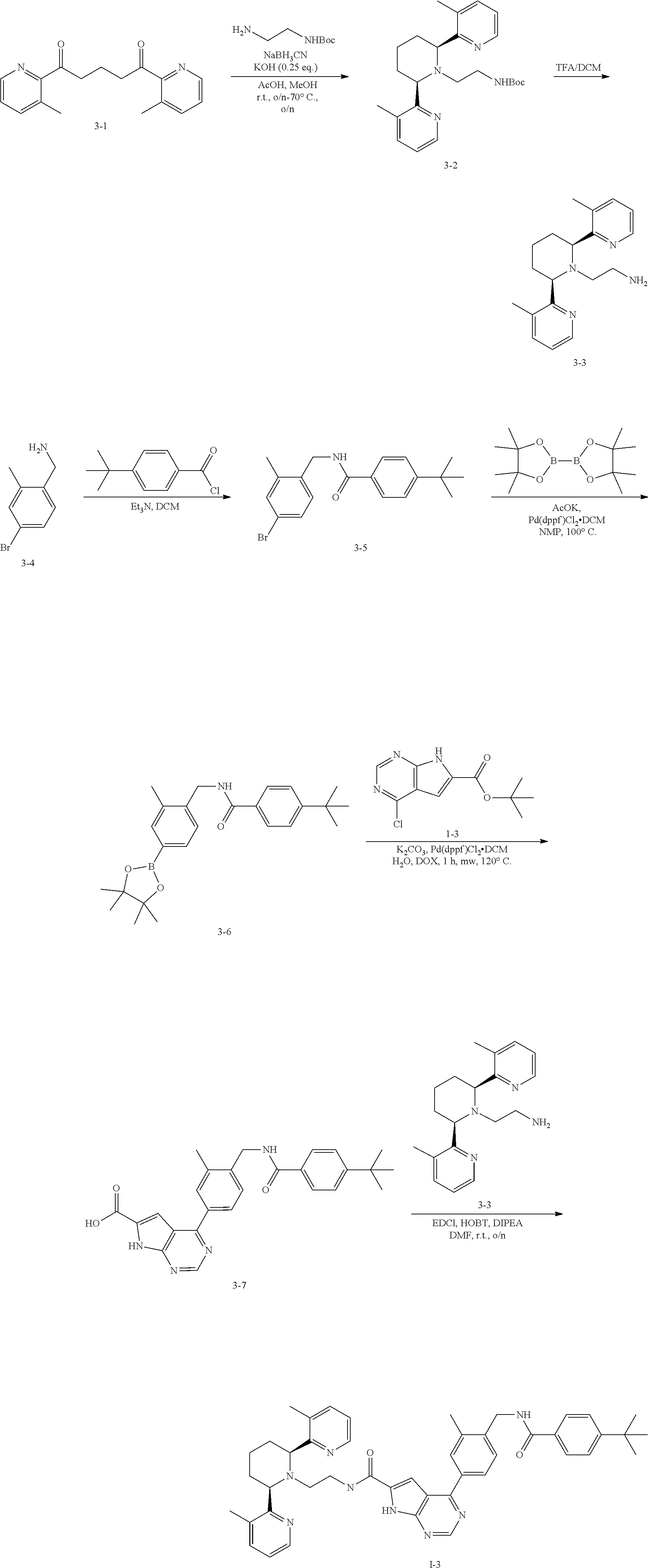

### 3.1 the Synthesis of 3-2

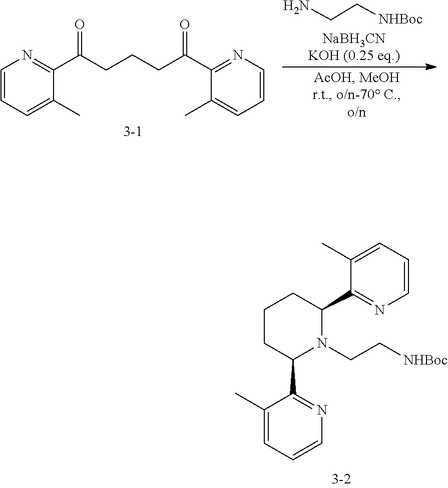 [SMILES: CC(C)(C)OC(=O)NCCN ; Cc1cccnc1C(=O)CCCC(=O)c1ncccc1C ; Cc1cccnc1[C@H]1CCC[C@@H](c2ncccc2C)N1CCNC(=O)OC(C)(C)C]

To a solution of 1,5-bis(3-methylpyridin-2-yl) pentane-1,5-dione (3-1, 500 mg, 1.77 mmol), tert-butyl 2-aminoethylcarbamate (567 mg, 3.55 mmol), potassium hydroxide (25 mg, 0.04 mmol) and AcOH (117 mg, 1.95 mmol) in CH₃OH (15 mL) was added NaBH₃CN (335 mg, 5.32 mmol), the mixture was stirred at room temperature overnight, then the mixture was stirred at 70° C. overnight After completion of the reaction indicated by LCMS, the reaction mixture was alkalixed to pH=8 with NaHCO₃ aqueous solution and concentrated to give the residue, which was purified by column chromatography to give tert-butyl 2-((2R,6S)-2,6-bis(3-methylpyridin-2-yl)piperidin-1-yl)ethylcarbamate (3-2, 400 mg, 55%) as yellow solid.

LCMS (Agilent LCMS 1200-6120, Column: Waters X-Bridge C18 (50 mm*4.6 mm*3.5 μm); Column Temperature: 40° C.; Flow Rate: 2.0 mL/min; Mobile Phase: from 95% [water+10 mM NH₄HCO₃] and 5% [CH₃CN] to 0% [water+10 mM NH₄HCO₃] and 100% [CH₃CN] in 1.6 min, then under this condition for 1.4 min, finally changed to 95% [water+10 mM NH₄HCO₃] and 5% [CH₃CN] in 0.1 min and under this condition for 0.7 min.) Purity: 66.16%, Rt=1.875 min; MS Calcd.: 410.3; MS Found: 411.2 [M+H]⁺.

### 3.2 the Synthesis of 3-3

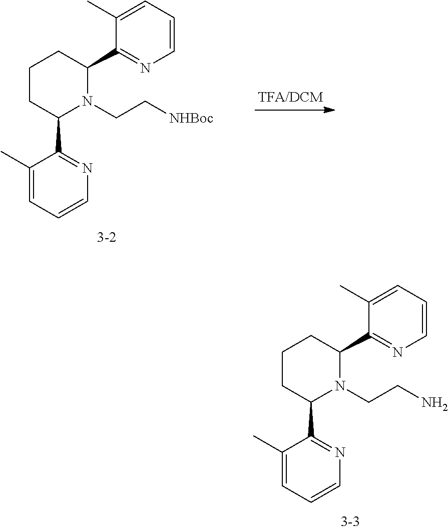 [SMILES: Cc1cccnc1[C@H]1CCC[C@@H](c2ncccc2C)N1CCNC(=O)OC(C)(C)C ; Cc1cccnc1[C@H]1CCC[C@@H](c2ncccc2C)N1CCN]

To a solution of tert-butyl 2-((2R, 6S)-2,6-bis(3-methylpyridin-2-yl)piperidin-1-yl)ethylcarbamate (3-2, 400 mg, 0.98 mol) in TFA (5 ml)/DCM (5 ml), and the mixture was stirred at room temperature overnight. After completion of the reaction indicated by LCMS, the reaction mixture was concentrated, neutralized with NaHCO₃ aq., and extracted with dichloromethane. The combined organic layers were washed with brine, dried over anhydrous Na₂SO₄, filtered and concentrated in vacuo to give the residue, which was purified by column chromatography and pre-HPLC to give 2-((2R,6S)-2,6-bis(3-methylpyridin-2-yl)piperidin-1-yl) ethanamine (3-3, 290 mg, 96%), as yellow solid. LCMS (Agilent LCMS 1200-6120, Column: Waters X-Bridge C18 (50 mm*4.6 mm*3.5 μm); Column Temperature: 40° C.; Flow Rate: 2.0 mL/min; Mobile Phase: from 95% [water+10 mM NH₄HCO₃] and 5% [CH₃CN] to 0% [water+10 mM NH₄HCO₃] and 100% [CH₃CN] in 1.6 min, then under this condition for 1.4 min, finally changed to 95% [water+10 mM NH₄HCO₃] and 5% [CH₃CN] in 0.1 min and under this condition for 0.7 min.) Purity: 98.09%, Rt=1.627 min; MS Calcd.: 310.2; MS Found: 311.2 [M+H]⁺.

### 3.3 the Synthesis of 3-5

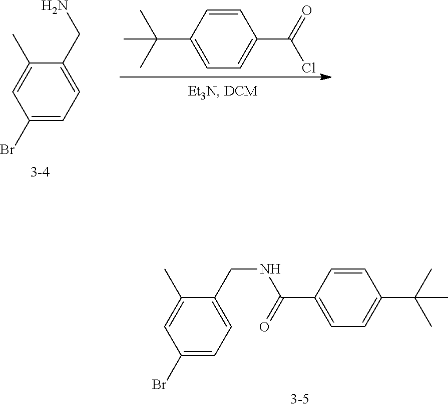

To a solution of (4-bromo-2-methylphenyl) methanamine (3-4, 4.5 g, 22.491 mmol), 4-tert-butylbenzoyl chloride (4.42 g, 22.491 mmol) in DCM (80 mL) was added riethylamine (4.55 g, 44.982 mmol) at room temperature for overnight. After completion of the reaction indicated by LCMS, the mixture was quenched with water, extracted with DCM (150 mL×3). The combined organic layers were washed with brine, dried over anhydrous Na₂SO₄, filtered and concentrated in vacuum to give a residue, which was purified by column chromatography (DCM:MeOH=20:1) to give N-(4-bromo-2-methylbenzyl)-4-tert-butylbenzamide (3-5, 7.2 g, 89%) as white solid.

### 3.4 the Synthesis of 3-6

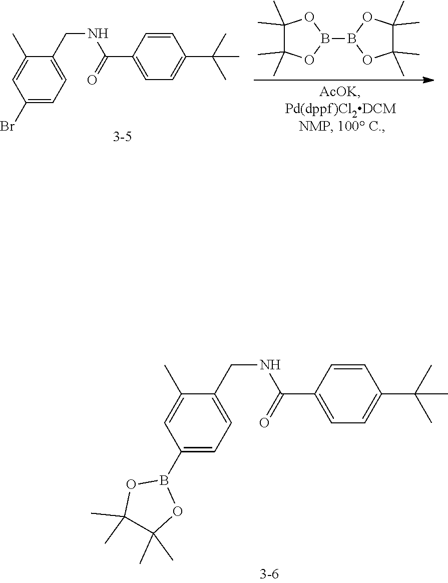

A solution of N-(4-bromobenzyl)-4-tert-butylbenzamide (3-5, 3.0 g, 8.354 mmol), 4,4,4′,4′,5,5,5′,5′-octamethyl-2,2′-bi (1,3,2-dioxaborolane) (2.54 g, 10.025 mmol), AcOK (2.46 g, 25.063 mmol), Pd(dppf)Cl₂·DCM (683 mg, 0.835 mmol) in NMP (60 mL) under N₂ at room temperature was stirred at 100° C. for overnight. After completion of the reaction indicated by LCMS, the mixture was poured into water (80 mL) and extracted with EA (60 mL×3), the organic layer was dried over Na₂SO₄, filtered and concentrated in vacuum to give a residue, which was purified by column chromatography (PE:EA=4:1) to give 4-tert-butyl-N-(2-methyl-4-(4,4,5,5-tetramethyl-1,3,2-dioxaborolan-2-yl)benzyl)benzamide (3-6, 1. 1 g, 32%) as white solid.

### 3.5 the Synthesis of 3-7

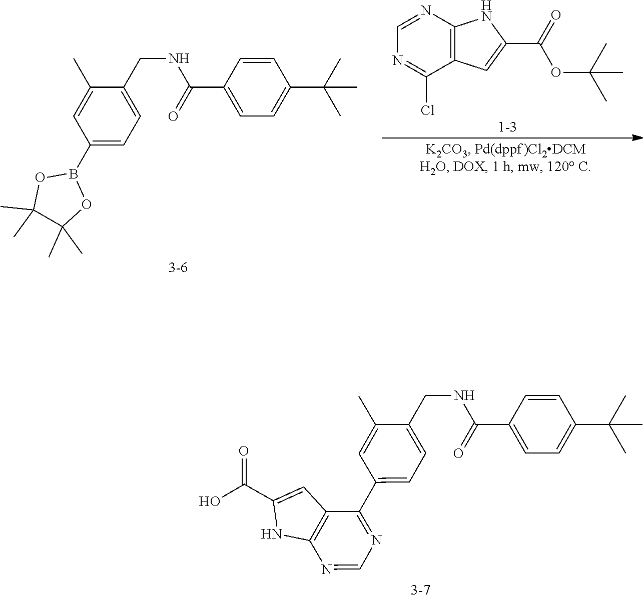 [SMILES: Cc1cc(B2OC(C)(C)C(C)(C)O2)ccc1CNC(=O)c1ccc(C(C)(C)C)cc1 ; CC(C)(C)OC(=O)c1cc2c(Cl)ncnc2[nH]1 ; Cc1cc(-c2ncnc3[nH]c(C(=O)O)cc23)ccc1CNC(=O)c1ccc(C(C)(C)C)cc1]

A solution of 4-tert-butyl-N-(2-methyl-4-(4,4,5,5-tetramethyl-1,3,2-dioxaborolan-2-yl)benzyl)benzamide (3-6, 900 mg, 2.210 mmol), tert-butyl 4-chloro-7H-pyrrolo[2,3-d]pyrimidine-6-carboxylate (1-3, 559 mg, 2.210 mmol), K₂CO₃ (916 mg, 6.630 mmol), Pd(dppf)Cl₂·DCM (90 mg, 0.110 mmol) in DOX (10 mL) and water (10 mL) was heated in the microwave at 120° C. for 1 h. After cooling to room temperature the mixture was concentrated to give the residue, the crude material was purified by flash column chromatography to give 4-(4-((4-tert-butylbenzamido)methyl)-3-methylphenyl)-7H-pyrrolo[2,3-d]pyrimidine-6-carboxylic acid (3-7, 430 mg, 44%) as a brown solid. LCMS (Agilent LCMS 1200-6120, Column: Waters X-Bridge C18 (50 mm*4.6 mm*3.5 μm); Column Temperature: 40° C.; Flow Rate: 2.0 mL/min; Mobile Phase: from 95% [water+10 mM NH₄HCO₃] and 5% [CH₃CN] to 0% [water+10 mM NH₄HCO₃] and 100% [CH₃CN] in 1.6 min, then under this condition for 1.4 min, finally changed to 95% [water+10 mM NH₄HCO₃] and 5% [CH₃CN] in 0.1 min and under this condition for 0.7 min.) Purity: 100.00%. Rt=1.535 min; MS Calcd.: 442.2; MS Found: 443.1 [M+H]⁺. HPLC (Agilent HPLC 1200, Column: L-column2 ODS (150 mm*4.6 mm*5.0 μm); Column Temperature: 40° C.; Flow Rate: 1.0 mL/min; Mobile Phase: from 90% [(total 10 mM AcONH₄) H₂O/MeCN=900/100 (v/v)] and 10% [total 10 mM AcONH₄) H₂O/MeCN=100/900 (v/v)] to 15% [total 10 mM AcONH₄) H₂O/MeCN=900/100 (v/v)] and 85% [total 10 mM AcONH₄) H₂O/MeCN=100/900 (v/v)] in 5 min, then under this condition for 10 min, finally changed to 90% [(total 10 mM AcONH₄) H₂O/MeCN=900/100 (v/v)] and 10% [total 10 mM AcONH₄) H₂O/MeCN=100/900 (v/v)] in 0.1 min and under this condition for 5 min. Purity: 100.00%, Rt=7.047 min; MS Calcd.: 442.2; MS Found: 443.0 [M+H]⁺. ¹H NMR (400 MHZ, DMSO-d₆) δ: 12.81 (s, 1H), 8.97 (t, J=6.0 Hz, 1H), 8.91 (s, 1H), 7.99 (d, J=7.6 Hz, 2H), 7.88 (d, J=8.4 Hz, 2H), 7.51 (d, J=8.4 Hz, 2H), 7.45 (d, J=8.0 Hz, 1H), 7.39 (s, 1H), 4.55 (d, J=5.2 Hz, 2H), 2.47 (s, 3H), 1.31 (s, 9H).

### 3.6 the Synthesis of Compound I-3

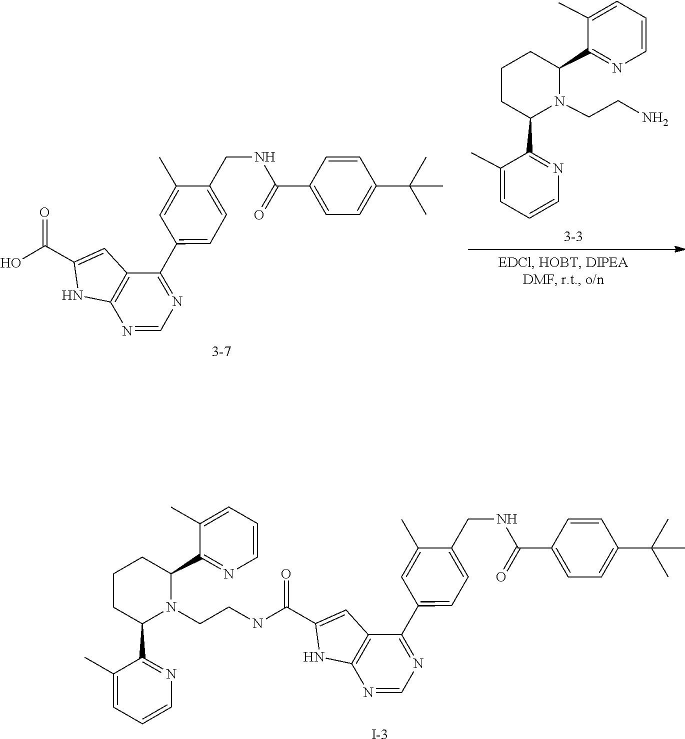

A solution of 4-(4-((4-tert-butylbenzamido)methyl)-3-methylphenyl)-7H-pyrrolo[2,3-d]pyrimidine-6-carboxylic acid (3-7, 100 mg, 0.226 mmol), 2-((2R,6S)-2,6-bis(3-methylpyridin-2-yl)piperidin-1-yl) ethanamine (3-3, 70 mg, 0.226 mmol), EDCI (65 mg, 0.339 mmol), DIPEA (88 mg, 0.678 mmol) and HOBT (46 mg, 0.339 mmol) in DMF (10 mL) was stirred at room temperature for overnight. After completion of the reaction indicated by LCMS, the mixture was concentrated in vacuum to give the residue, which was purified by prep-HPLC to give I-3 (70.65 mg, 42%) as off-white solid. LCMS (Agilent LCMS 1200-6120, Column: Waters X-Bridge C18 (50 mm*4.6 mm*3.5 μm); Column Temperature: 40° C.; Flow Rate: 2.0 mL/min; Mobile Phase: from 95% [water+10 mM NH₄HCO₃] and 5% [CH₃CN] to 0% [water+10 mM NH₄HCO₃] and 100% [CH₃CN] in 1.6 min, then under this condition for 1.4 min, finally changed to 95% [water+10 mM NH₄HCO₃] and 5% [CH₃CN] in 0.1 min and under this condition for 0.7 min.) Purity: 98.95%. Rt=2.099 min; MS Calcd.: 734.4; MS Found: 735.0 [M+H]⁺. HPLC (Agilent HPLC 1200, Column: L-column2 ODS (150 mm*4.6 mm*5.0 μm); Column Temperature: 40° C.; Flow Rate: 1.0 mL/min; Mobile Phase: from 90% [(total 10 mM AcONH₄) H₂O/MeCN=900/100 (v/v)] and 10% [total 10 mM AcONH₄) H₂O/MeCN=100/900 (v/v)] to 15% [total 10 mM AcONH₄) H₂O/MeCN=900/100 (v/v)] and 85% [total 10 mM AcONH₄) H₂O/MeCN=100/900 (v/v)] in 5 min, then under this condition for 10 min, finally changed to 90% [(total 10 mM AcONH₄) H₂O/MeCN=900/100 (v/v)] and 10% [total 10 mM AcONH₄) H₂O/MeCN=100/900 (v/v)] in 0.1 min and under this condition for 5 min. Purity: 99.26%, Rt=11.223 min; MS Calcd.: 734.4; MS Found: 735.4 [M+H]⁺. ¹H NMR (400 MHZ, DMSO-d₆): 12.47 (s, 1H), 8.99 (t, J=5.6 Hz, 1H), 8.84 (s, 1H), 8.31 (d, J=4.0 Hz, 1H), 7.96-7.88 (m, 5H), 7.52 (d, J=8.4 Hz, 2H), 7.43 (d, J=4.0 Hz, 2H), 7.37 (s, 1H), 7.07 (t, J=5.2 Hz, 2H), 4.55 (d, J=5.2 Hz, 2H), 4.26 (d, J=8.0 Hz, 2H), 2.55 (s, 8H), 2.45 (s, 3H), 2.15-2.08 (m, 4H), 1.97-1.94 (m, 1H), 1.68-1.61 (m, 1H), 1.55-1.48 (m, 2H), 1.32 (s, 9H).

### Example 4: Synthesis of Compound I-4

### Synthetic Scheme of Compound I-4

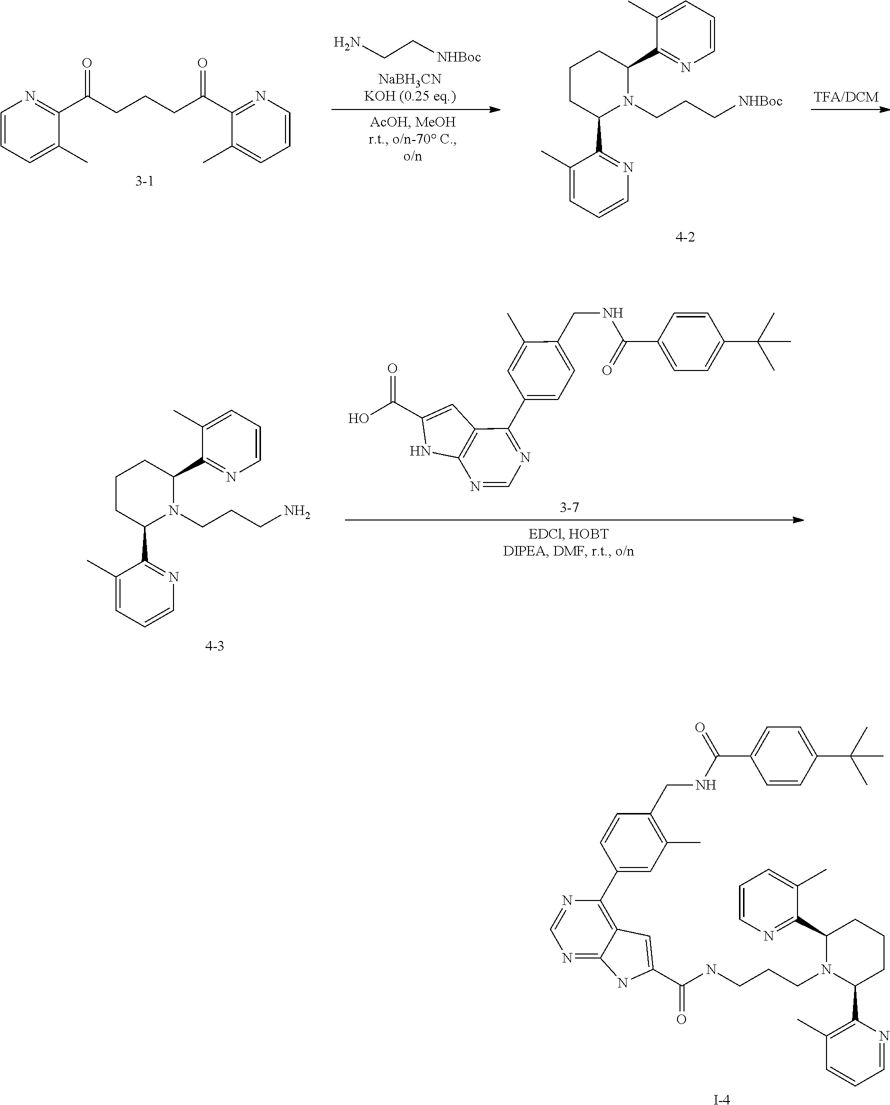

### 4.1 The Synthesis of 4-2

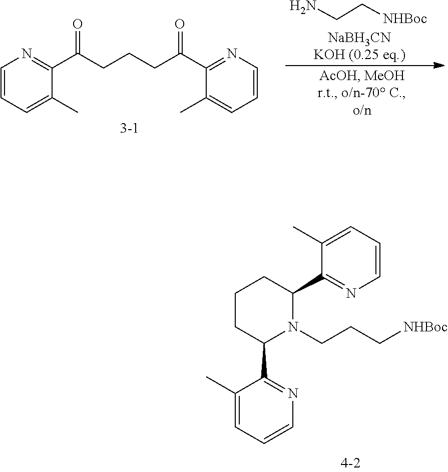 [SMILES: CC(C)(C)OC(=O)NCCN ; Cc1cccnc1C(=O)CCCC(=O)c1ncccc1C ; Cc1cccnc1[C@H]1CCC[C@@H](c2ncccc2C)N1CCCNC(=O)OC(C)(C)C]

To a solution of 1,5-bis(3-methylpyridin-2-yl) pentane-1,5-dione (3-1, 500 mg, 1.771 mmol) in MeOH (20 mL) was added KOH (25 mg, 0.443 mmol), tert-butyl tert-butyl 3-aminopropylcarbamate (926 mg, 5.313 mmol), AcOH (117 mg, 1.948 mmol) and NaBH₃CN (167 mg, 2.656 mmol), then the mixture was stirred at room temperature for overnight, followed by stirring at 70° C. for overnight. After completion of the reaction indicated by LCMS, the mixture was poured into water (15 mL) and extracted with DCM (40 mL×3). The organic layer was dried over Na₂SO₄, filtered and concentrated in vacuum to give a residue, which was purified by column chromatography (PE:EA=2:1) to give the desired product (4-2, 470 mg, 62%) as yellow solid.

### 4.2 the Synthesis of 4-3

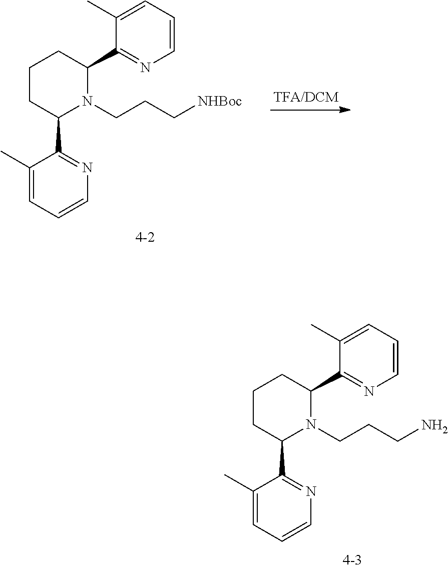 [SMILES: Cc1cccnc1[C@H]1CCC[C@@H](c2ncccc2C)N1CCCNC(=O)OC(C)(C)C ; Cc1cccnc1[C@H]1CCC[C@@H](c2ncccc2C)N1CCCN]

To a solution of tert-butyl 3-((2R,6S)-2,6-bis(3-methylpyridin-2-yl)piperidin-1-yl) propylcarbamate (4-2, 470 mg, 1.107 mmol) in dry DCM (10 mL) was added TFA (5 mL), then the mixture was stirred at room temperature for overnight. After completion of the reaction indicated by LCMS, the mixture was concentrated in vacuum, diluted with DCM, adjusted to pH=9 by sat. NaHCO₃ solution and extracted with DCM/MeOH=30:1 (20 mL×3). The organic layer was dried over Na₂SO₄, filtered and concentrated in vacuum to give a residue, which was purified by column chromatography (DCM:MeOH=20:1) to give the desired product (4-3, 310 mg, 86%) as yellow oil.

### 4.3 the Synthesis of Compound I-4

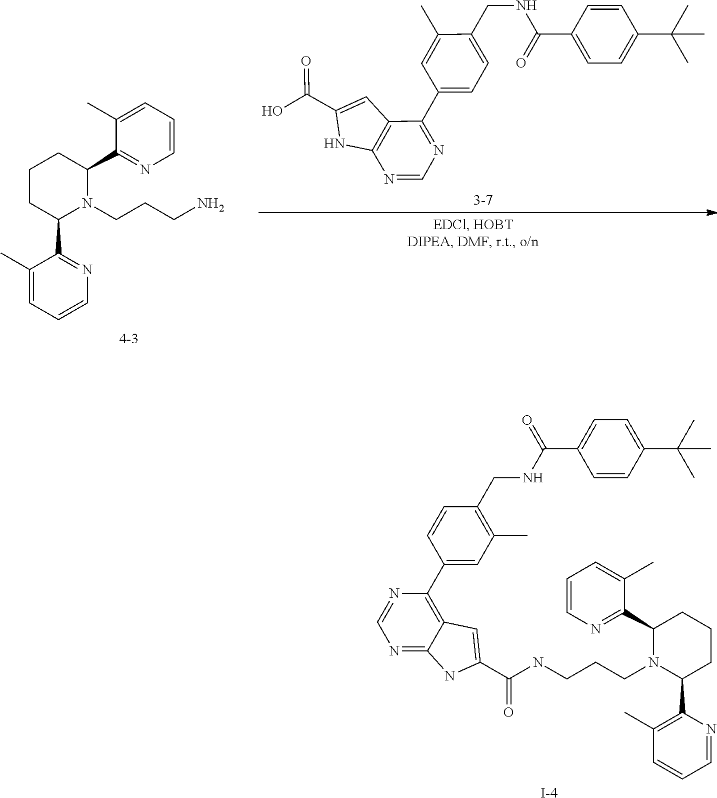

A solution of 4-(4-((4-tert-butylbenzamido)methyl)-3-methylphenyl)-7H-pyrrolo[2,3-d]pyrimidine-6-carboxylic acid (3-7, 100 mg, 0.226 mmol), 3-((2R,6S)-2,6-bis(3-methylpyridin-2-yl)piperidin-1-yl) propan-1-amine (4-3, 73 mg, 0.226 mmol), EDCI (65 mg, 0.339 mmol), DIPEA (88 mg, 0.678 mmol) and HOBT (46 mg, 0.339 mmol) in DMF (10 Ml) was stirred at room temperature for overnight. After completion of the reaction indicated by LCMS, the mixture was concentrated in vacuum to give the residue, which was purified by prep-HPLC to give I-4 (66.31 mg, 39%) as off-white solid. LCMS (Agilent LCMS 1200-6120, Column: Waters X-Bridge C18 (50 mm*4.6 mm*3.5 μm); Column Temperature: 40° C.; Flow Rate: 2.0 mL/min; Mobile Phase: from 95% [water+10 mM NH₄HCO₃] and 5% [CH₃CN] to 0% [water+10 mM NH₄HCO₃] and 100% [CH₃CN] in 1.6 min, then under this condition for 1.4 min, finally changed to 95% [water+10 mM NH₄HCO₃] and 5% [CH₃CN] in 0.1 min and under this condition for 0.7 min.) Purity: 100.00%. Rt=2.245 min; MS Calcd.: 748.4; MS Found: 749.4 [M+H]⁺. HPLC (Agilent HPLC 1200, Column: L-column2 ODS (150 mm*4.6 mm*5.0 μm); Column Temperature: 40° C.; Flow Rate: 1.0 mL/min; Mobile Phase: from 90% [(total 10 mM AcONH₄) H₂O/MeCN=900/100 (v/v)] and 10% [total 10 mM AcONH₄) H₂O/MeCN=100/900 (v/v)] to 15% [total 10 mM AcONH₄) H₂O/MeCN=900/100 (v/v)] and 85% [total 10 mM AcONH₄) H₂O/MeCN=100/900 (v/v)] in 5 min, then under this condition for 10 min, finally changed to 90% [(total 10 mM AcONH₄) H₂O/MeCN=900/100 (v/v)] and 10% [total 10 mM AcONH₄) H₂O/MeCN=100/900 (v/v)] in 0.1 min and under this condition for 5 min. Purity: 100.00%, Rt=10.257 min; MS Calcd.: 748.4; MS Found: 749.0 [M+H]⁺. ¹H NMR (400 MHZ, DMSO-d₆) δ: 12.53 (s, 1H), 9.00 (t, J=5.0 Hz, 1H), 8.86 (s, 1H), 8.20 (d, J=6.0 Hz, 3H), 7.98-7.88 (m, 4H), 7.52 (d, J=8.4 Hz, 2H), 7.45-7.37 (m, 4H), 6.95 (s, 2H), 4.57 (d, J=5.6 Hz, 2H), 2.67 (s, 6H), 2.51 (s, 3H), 2.48 (s, 2H), 2.39-2.33 (m, 2H), 2.08 (s, 2H), 1.91-1.89 (m, 1H), 1.61-1.58 (m, 1H), 1.50-1.48 (m, 2H), 1.32 (s, 9H), 0.61-0.58 (m, 2H).

### Example 5: Synthesis of Compound I-5

### Synthetic Scheme of Compound I-5

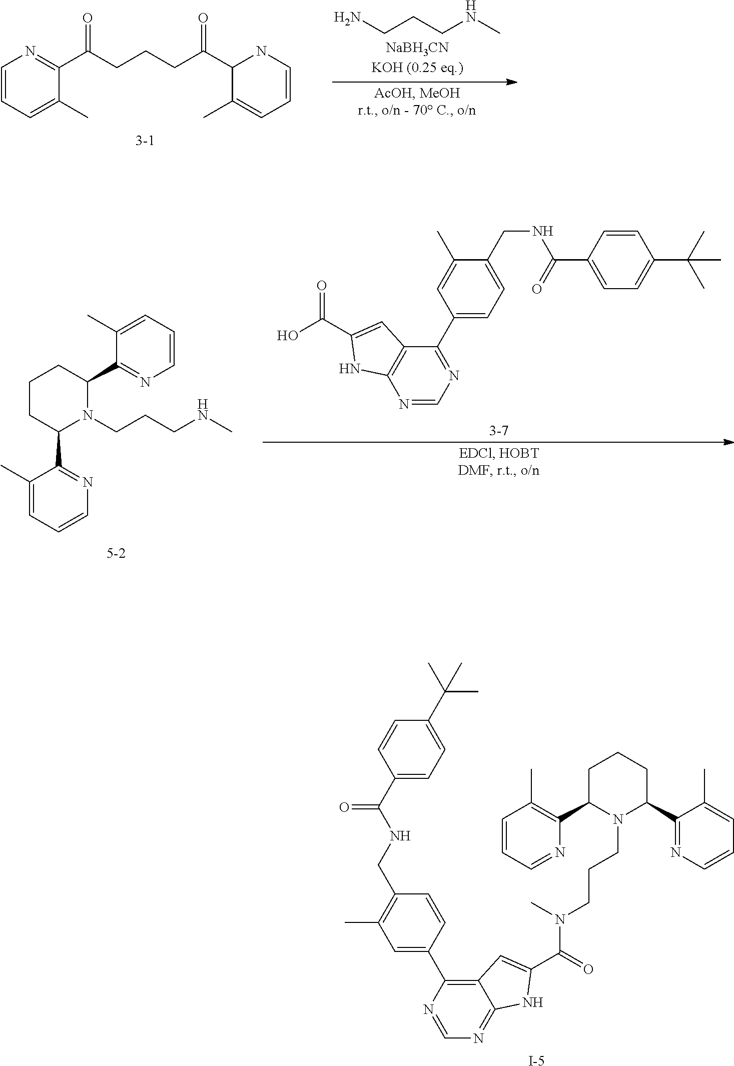

### 5.1 The Synthesis of 5-2

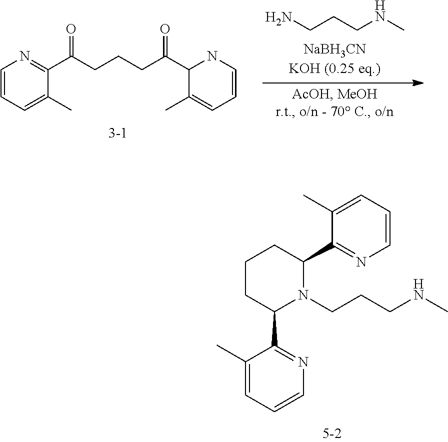

To a solution of 1,5-bis(3-methylpyridin-2-yl) pentane-1,5-dione (3-1, 600 mg, 2.13 mmol), N₁-methylpropane-1,3-diamine (374 mg, 4.26 mmol), potassium hydroxide (30 mg, 0.53 mmol) and AcOH (140 mg, 2.34 mmol) in CH₃OH (15 mL) was added NaBH₃CN (402 mg, 6.38 mmol), the mixture was stirred at room temperature overnight, then the mixture was stirred at 70° C. overnight After completion of the reaction indicated by LCMS, the reaction mixture was alkalized to pH=8 with NaHCO₃ aqueous solution and concentrated to give the residue, which was purified by column chromatography to give 3-((2R,6S)-2,6-bis(3-methylpyridin-2-yl)piperidin-1-yl)-N-methylpropan-1-amine (5-2, 140 mg, 19%) as yellow solid.

LCMS (Agilent LCMS 1200-6120, Column: Waters X-Bridge C18 (50 mm*4.6 mm*3.5 μm)); Column Temperature: 40° C.; Flow Rate: 2.3 mL/min; Mobile Phase: from 95% [water+10 mM NH₄HCO₃] and 5% [CH₃CN] to 5% [water+10 mM NH₄HCO₃] and 100% [CH₃CN] in 1.75 min, then under this condition for 0.8 min, finally changed to 95% [water+10 mM NH₄HCO₃] and 5% [CH₃CN] in 0.1 min and under this condition for 0.1 min. Purity: 76.49%, Rt=1.875 min; MS Calcd.: 338.2; MS Found: 339.1 [M+H]⁺.

### 5.2 the Synthesis of Compound I-5

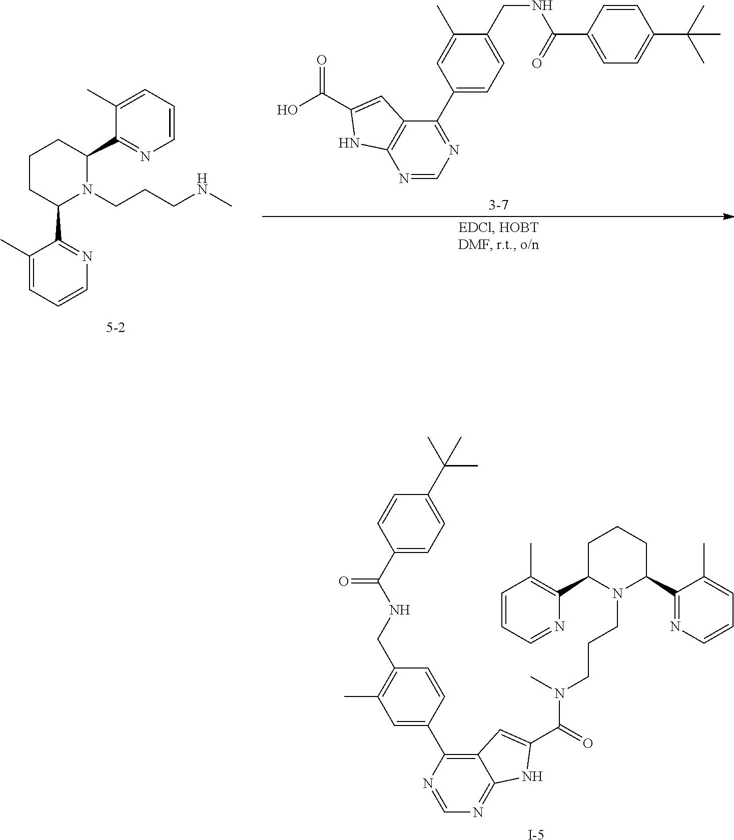

A solution of 4-(4-((4-tert-butylbenzamido)methyl)-3-methylphenyl)-7H-pyrrolo[2,3-d]pyrimidine-6-carboxylic acid (3-7, 100 mg, 0.226 mmol), 3-((2R,6S)-2,6-bis(3-methylpyridin-2-yl)piperidin-1-yl)-N-methylpropan-1-amine (5-2, 76.38 mg, 0.226 mmol), EDCI (65 mg, 0.339 mmol), DIPEA (88 mg, 0.678 mmol) and HOBT (46 mg, 0.339 mmol) in DMF (10 mL) was stirred at room temperature for overnight. After completion of the reaction indicated by LCMS, the mixture was concentrated in vacuum to give the residue, which was purified by prep-HPLC to give I-5 (55.31 mg, 34%) as off-white solid. LCMS (Agilent LCMS 1200-6120, Column: Waters X-Bridge C18 (50 mm*4.6 mm*3.5 μm); Column Temperature: 40° C.; Flow Rate: 2.0 mL/min; Mobile Phase: from 95% [water+10 mM NH₄HCO₃] and 5% [CH₃CN] to 0% [water+10 mM NH₄HCO₃] and 100% [CH₃CN] in 1.6 min, then under this condition for 1.4 min, finally changed to 95% [water+10 mM NH₄HCO₃] and 5% [CH₃CN] in 0.1 min and under this condition for 0.7 min.) Purity: 100.00%. Rt=2.01 min; MS Calcd.: 762.4; MS Found: 763.0 [M+H]⁺. HPLC (Agilent HPLC 1200, Column: L-column2 ODS (150 mm*4.6 mm*5.0 μm); Column Temperature: 40° C.; Flow Rate: 1.0 mL/min; Mobile Phase: from 90% [(total 10 mM AcONH₄) H₂O/MeCN=900/100 (v/v)] and 10% [total 10 mM AcONH₄) H₂O/MeCN=100/900 (v/v)] to 15% [total 10 mM AcONH₄) H₂O/MeCN=900/100 (v/v)] and 85% [total 10 mM AcONH₄) H₂O/MeCN=100/900 (v/v)] in 5 min, then under this condition for 10 min, finally changed to 90% [(total 10 mM AcONH₄) H₂O/MeCN=900/100 (v/v)] and 10% [total 10 mM AcONH₄) H₂O/MeCN=100/900 (v/v)] in 0.1 min and under this condition for 5 min. Purity: 100.00%, Rt=10.30 min; MS Calcd.: 762.4; MS Found: 763.0 [M+H]⁺. ¹H NMR (400 MHZ, DMSO-d₆) ô: 12.28 (s, 1H), 8.85 (s, 1H), 8.76 (t, J=5.6 Hz, 1H), 8.15 (d, J=6.0 Hz, 2H), 7.95-7.92 (m, 2H), 7.88-7.84 (m, 2H), 7.50-7.45 (m, 3H), 7.33 (s, 2H), 6.97 (s, 2H), 6.90 (s, 2H), 4.65 (d, J=5.6 Hz, 2H), 4.09-4.03 (m, 2H), 2.64-2.57 (m, 8H), 2.47 (s, 6H), 2.42-2.39 (m, 2H), 2.12-2.07 (m, 2H), 2.02-2.00 (m, 1H), 1.88-1.65 (m, 3H), 1.35 (s, 9H), 1.32 (s, 9H), 0.73-0.52 (m, 2H).

### Example 6: Synthesis of Compound I-6

### 6.1 the Synthesis of Compound I-6

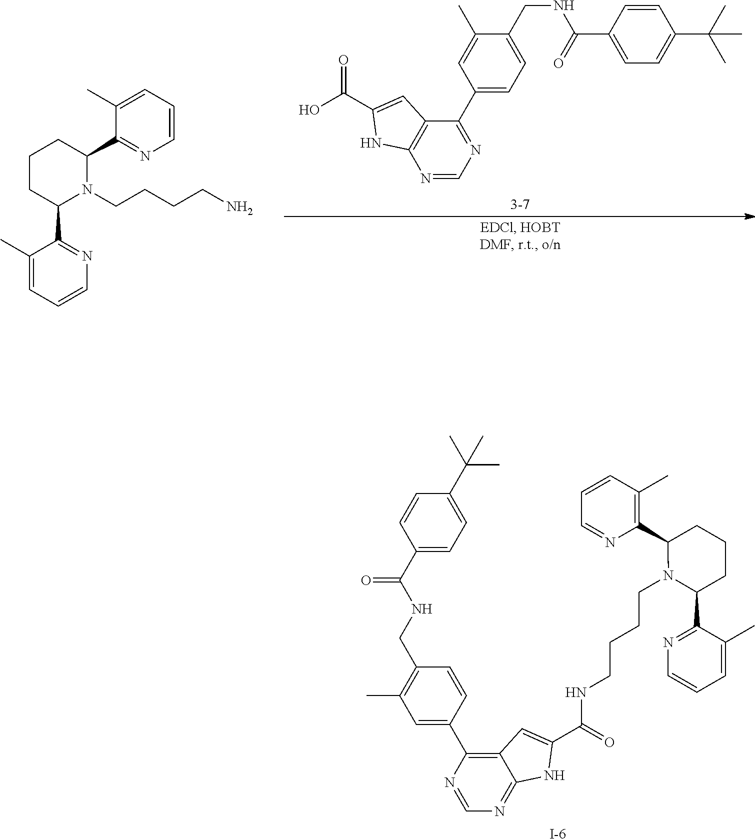

A solution of 4-(4-((4-tert-butylbenzamido)methyl)-3-methylphenyl)-7H-pyrrolo[2,3-d]pyrimidine-6-carboxylic acid (3-7, 100 mg, 0.226 mmol), 4-((2R,6S)-2,6-bis(3-methylpyridin-2-yl)piperidin-1-yl) butan-1-amine (76.38 mg, 0.226 mmol), EDCI (65 mg, 0.339 mmol), DIPEA (88 mg, 0.678 mmol) and HOBT (46 mg, 0.339 mmol) in DMF (10 mL) was stirred at room temperature for overnight. After completion of the reaction indicated by LCMS, the mixture was concentrated in vacuum to give the residue, which was purified by prep-HPLC to give I-6 (52.33 mg, 32%) as off-white solid. LCMS (Agilent LCMS 1200-6120, Column: Waters X-Bridge C18 (50 mm*4.6 mm*3.5 μm); Column Temperature: 40° C.; Flow Rate: 2.0 mL/min; Mobile Phase: from 95% [water+10 mM NH₄HCO₃] and 5% [CH₃CN] to 0% [water+10 mM NH₄HCO₃] and 100% [CH₃CN] in 1.6 min, then under this condition for 1.4 min, finally changed to 95% [water+10 mM NH₄HCO₃] and 5% [CH₃CN] in 0.1 min and under this condition for 0.7 min.) Purity: 100.00%. Rt=2.05 min; MS Calcd.: 762.4; MS Found: 763.1 [M+H]⁺. HPLC (Agilent HPLC 1200, Column: L-column2 ODS (150 mm*4.6 mm*5.0 μm); Column Temperature: 40° C.; Flow Rate: 1.0 mL/min; Mobile Phase: from 90% [(total 10 mM AcONH₄) H₂O/MeCN=900/100 (v/v)] and 10% [total 10 mM AcONH₄) H₂O/MeCN=100/900 (v/v)] to 15% [total 10 mM AcONH₄) H₂O/MeCN=900/100 (v/v)] and 85% [total 10 mM AcONH₄) H₂O/MeCN=100/900 (v/v)] in 5 min, then under this condition for 10 min, finally changed to 90% [(total 10 mM AcONH₄) H₂O/MeCN=900/100 (v/v)] and 10% [total 10 mM AcONH₄) H₂O/MeCN=100/900 (v/v)] in 0.1 min and under this condition for 5 min. Purity: 100.00%, Rt=10.55 min; MS Calcd.: 762.4; MS Found: 763.0 [M+H]⁺. ¹H NMR (400 MHZ, DMSO-d₆) δ: 12.51 (s, 1H), 9.00 (t, J=6.0 Hz, 1H), 8.87 (s, 1H), 8.29 (s, 3H), 7.99-7.88 (m, 4H), 7.53-7.39 (m, 6H), 7.02 (s, 2H), 4.56 (d, J=5.6 Hz, 2H), 4.18 (s, 2H), 2.67-2.61 (m, 5H), 2.47 (s, 6H), 2.42-2.39 (m, 2H), 2.12-2.07 (m, 2H), 2.02-2.00 (m, 1H), 1.88-1.65 (m, 3H), 1.35 (s, 9H), 1.32 (s, 9H), 0.66 (s, 2H), 0.29-0.27 (m, 2H).

### Example 7: Synthesis of Compound I-7

### Synthetic Scheme of Compound I-7

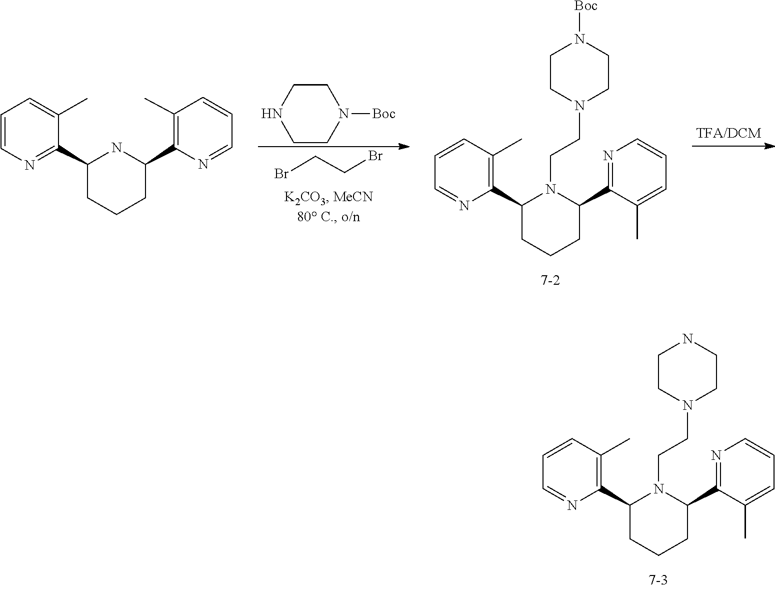

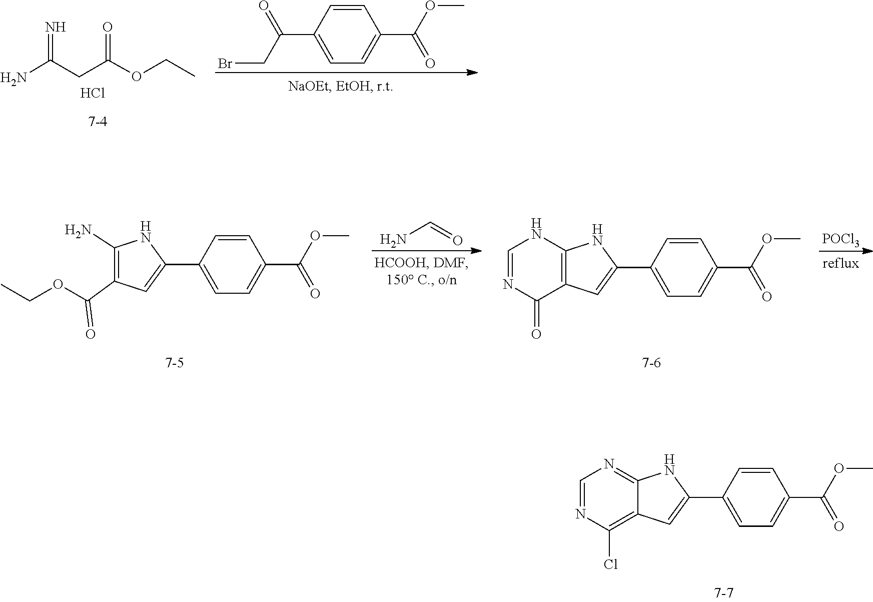

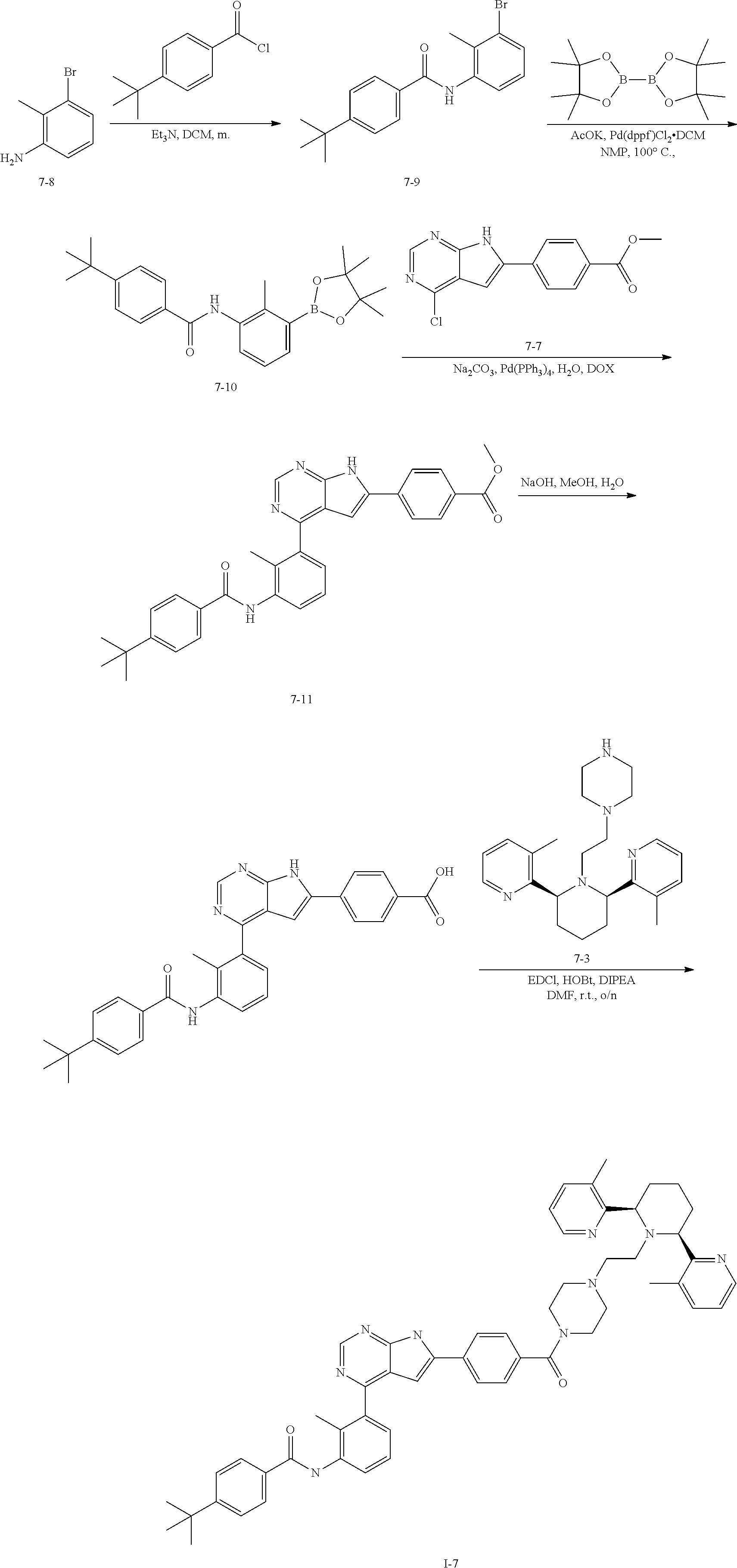

### The Synthesis of 7-2

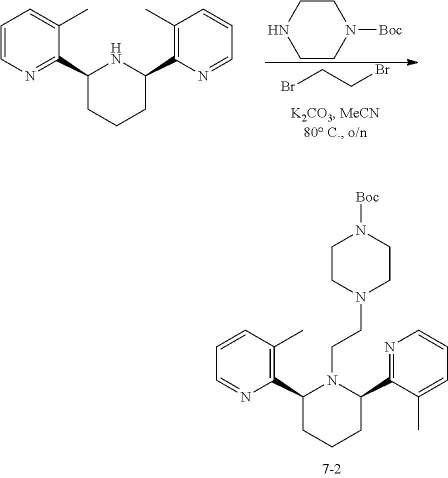

To a solution of (2R,6S)-2,6-bis(3-methylpyridin-2-yl)piperidine (1.0 g, 3.743 mmol) in MeCN (60 mL) was added tert-butyl piperazine-1-carboxylate (697 mg, 3.743 mmol), 1,2-dibromoethane (703 mg, 3.743 mmol) and K₂CO₃ (1.0 g, 7.486 mmol), then the mixture was stirred at 80° C. for overnight. After completion of the reaction indicated by LCMS, the mixture was filtered, poured into water and extracted with DCM (150 mL×3). The combined organic layers were washed with brine, dried over anhydrous Na₂SO₄, filtered and concentrated in vacuum to give a residue, which was purified by column chromatography (DCM:MeOH=10:1) to give N-(4-bromo-2-methylbenzyl)-4-tert-butylbenzamide (7-2, 330 mg, 18%) as yellow oil.

### 7.2 the Synthesis of 7-3

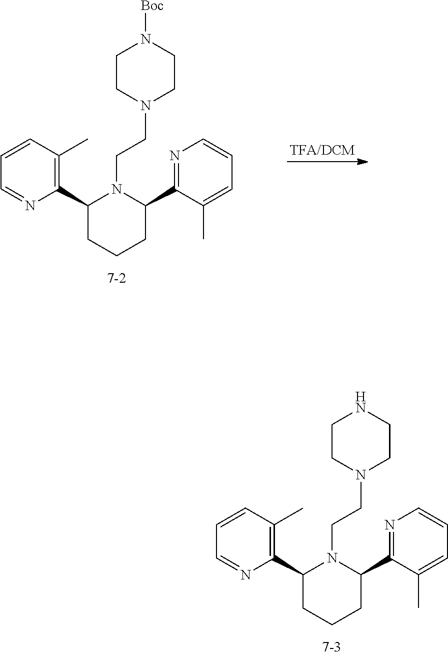 [SMILES: Cc1cccnc1[C@H]1CCC[C@@H](c2ncccc2C)N1CCN1CCN(C(=O)OC(C)(C)C)CC1 ; Cc1cccnc1[C@H]1CCC[C@@H](c2ncccc2C)N1CCN1CCNCC1]

To a solution of tert-butyl 4-(2-((2S,6R)-2,6-bis(3-methylpyridin-2-yl)piperidin-1-yl)ethyl)piperazine-1-carboxylate (7-2, 330 mg, 0.689 mmol) in dry DCM (8 mL) was added TFA (4 mL), then the mixture was stirred at room temperature overnight. After completion of the reaction indicated by LCMS, the mixture was concentrated in vacuum, diluted with water, adjusted to pH=9 by NaHCO₃ solution and extracted with DCM/MeOH=30:1 (20 mL×3). The organic layer was dried over Na₂SO₄, filtered and concentrated in vacuum to give a residue, which was purified by column chromatography (DCM:MeOH=5:1) to give 1-(2-((2S,6R)-2,6-bis(3-methylpyridin-2-yl)piperidin-1-yl)ethyl)piperazine (7-3, 170 mg, 65%) as yellow oil.

### 7.3 the Synthesis of 7-5

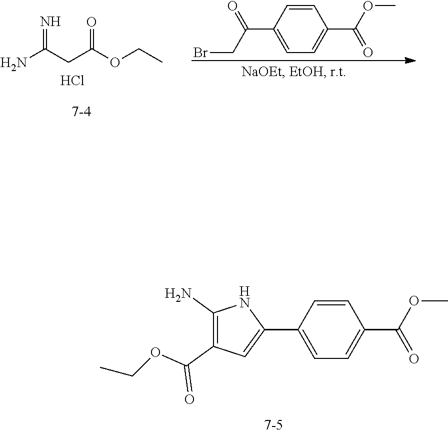

To a solution of ethyl 3-amino-3-iminopropanoate hydrochloride (7-4, 16.6 g, 100 mmol) and methyl 4-(2-bromoacetyl)benzoate (25.7 g, 100 mmol) in EtOH (500 mL) was added NaOEt (40 mL, 2.5 M, 100 mmol), then the mixture was stirred at room temperature overnight. After completion of the reaction indicated by LCMS, the mixture was filtered and the residue was washed with MeOH for 2 times. The combined organic solid were concentrated in vacuo to give 7-5 (8.9 g, 32%) as brown solid.

LCMS (Agilent LCMS 1200-6120, Column: Waters X-Bridge C18 (50 mm*4.6 mm*3.5 μm); Column Temperature: 40° C.; Flow Rate: 2.0 mL/min; Mobile Phase: from 95% [water+10 mM NH₄HCO₃] and 5% [CH₃CN] to 0% [water+10 mM NH₄HCO₃] and 100% [CH₃CN] in 1.6 min, then under this condition for 1.4 min, finally changed to 95% [water+10 mM NH₄HCO₃] and 5% [CH₃CN] in 0.1 min and under this condition for 0.7 min.) Purity: 88.27%. Rt=1.76 min; MS Calcd.: 288.1; MS Found: 289.0 [M+H]⁺.

### 7.4 the Synthesis of 7-6

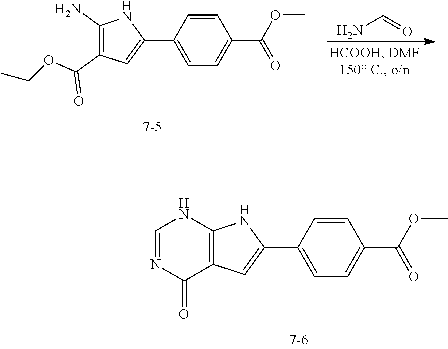

A solution of ethyl 2-amino-5-(4-(methoxycarbonyl)phenyl)-1H-pyrrole-3-carboxylate (7-5, 8.64 g, 30 mmol) in formamide (15 mL), HCOOH (5 mL) and DMF (3 mL) under N₂ at room temperature was stirred at 150° C. overnight. After cooling to room temperature, the mixture was diluted with IPA, the solid was filtered and concentrated to give 7-6 (7.1 g, 87% yield) as brown solid. LCMS (Agilent LCMS 1200-6110, Column: Waters X-Bridge C18 (50 mm*4.6 mm*3.5 μm); Column Temperature: 40° C.; Flow Rate: 1.5 mL/min; Mobile Phase: from 95% [water+0.05% TFA] and 5% [CH₃CN+0.05% TFA] to 0% [water+0.05% TFA] and 100% [CH₃CN+0.05% TFA] in 0.8 min, then under this condition for 0.4 min, finally changed to 95% [water+0.05% TFA] and 5% [CH₃CN+0.05% TFA] and under this condition for 0.01 min). Purity: 100.00%, Rt=1.38 min; MS Calcd.: 269.1; MS Found: 270.1 [M+H]⁺.

### 7.5 the Synthesis of 7-7

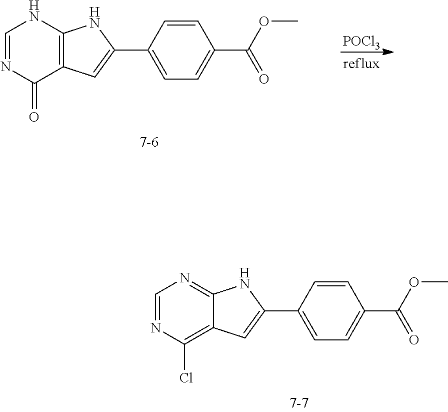

A solution of methyl 4-(4-oxo-4, 7-dihydro-1H-pyrrolo[2, 3-d]pyrimidin-6-yl)benzoate (7-6, 7.1 g, 26.4 mmol) in POCl₃ (100 mL) was stirred at 120° C. overnight. After completion of the reaction indicated by LCMS, the mixture was concentrated and diluted with sat. NHCO₃ solution, the solid was filtered and concentrated to give 7-7 (6.0 g, 80% yield) as brown solid. LCMS (Agilent LCMS 1200-6120, Column: Waters X-Bridge C18 (50 mm*4.6 mm*3.5 μm); Column Temperature: 40° C.; Flow Rate: 2.0 mL/min; Mobile Phase: from 95% [water+10 mM NH₄HCO₃] and 5% [CH₃CN] to 0% [water+10 mM NH₄HCO₃] and 100% [CH₃CN] in 1.6 min, then under this condition for 1.4 min, finally changed to 95% [water+10 mM NH₄HCO₃] and 5% [CH₃CN] in 0.1 min and under this condition for 0.7 min.) Purity: 94.58%. Rt=1.77 min; MS Calcd.: 287.1; MS Found: 288.1 [M+H]⁺.

### 7.6 the Synthesis of 7-9

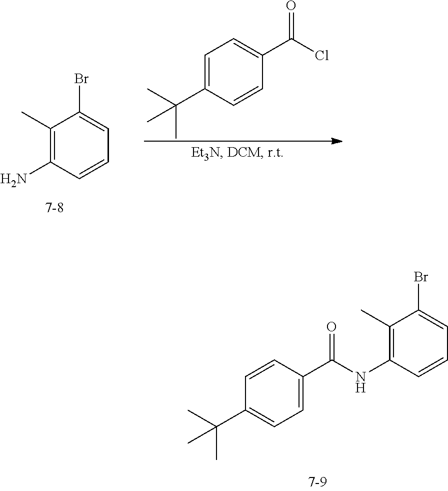

To a solution of 3-bromo-2-methylaniline (7-8, 3.0 g, 16.22 mmol), 4-tert-butylbenzoyl chloride (3.2 g, 16.22 mmol) in DCM (40 mL) was added Triethylamine (3.3 g, 32.44 mmol) at room temperature for overnight. After completion of the reaction indicated by LCMS, the mixture was quenched with water, extracted with dichloromethane (20 mL×3). The combined organic layers were washed with brine, dried over anhydrous Na₂SO₄, filtered and concentrated in vacuo to give the residue, which was purified by column chromatography to give N-(3-bromo-2-methylphenyl)benzamide (7-9, 3.2 g, 60%) as light yellow solid. LCMS (Agilent LCMS 1200-6120, Column: Waters X-Bridge C18 (50 mm*4.6 mm*3.5 μm); Column Temperature: 40° C.; Flow Rate: 2.0 mL/min; Mobile Phase: from 95% [water+10 mM NH₄HCO₃] and 5% [CH₃CN] to 0% [water+10 mM NH₄HCO₃] and 100% [CH₃CN] in 1.6 min, then under this condition for 1.4 min, finally changed to 95% [water+10 mM NH₄HCO₃] and 5% [CH₃CN] in 0.1 min and under this condition for 0.7 min.) Purity: 100.00%. Rt=1.94 min; MS Calcd.: 345.1; MS Found: 346.0 [M+H]⁻.

### 7.7 the Synthesis of 7-10

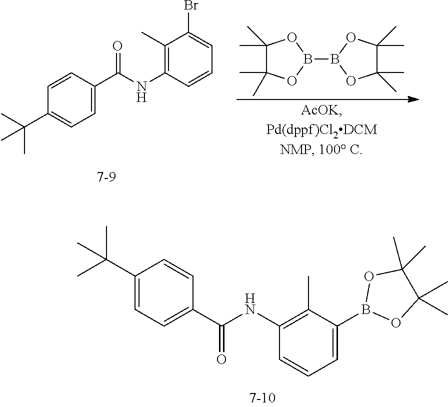

A solution of N-(3-bromo-2-methylphenyl)-4-tert-butylbenzamide (7-9, 2.4 g, 6.92 mmol), 4,4,4′,4′,5,5,5′,5′-octamethyl-2,2′-bi (1,3,2-dioxaborolane) (1.93 g, 7.62 mmol), AcOK (2.03 g, 20.76 mmol), Pd(dppf)Cl₂·DCM (559 mg, 0.69 mmol) in DOX (30 mL) under N₂ at room temperature was stirred at 100° C. for 16 h. After cooling to room temperature the mixture was diluted with EA and washed with water. The organic phase was dried over Na₂SO₄, filtered and concentrated. The crude material was purified by flash column chromatography on silica gel (PE:EA=10:1) to give 7-10 (2.0 g, 78% yield) as a white solid. LCMS (Agilent LCMS 1200-6110, Column: Waters X-Bridge C18 (50 mm*4.6 mm*3.5 μm); Column Temperature: 40° C.; Flow Rate: 1.5 mL/min; Mobile Phase: from 95% [water+0.05% TFA] and 5% [CH₃CN+0.05% TFA] to 0% [water+0.05% TFA] and 100% [CH₃CN+0.05% TFA] in 0.8 min, then under this condition for 0.4 min, finally changed to 95% [water+0.05% TFA] and 5% [CH₃CN+0.05% TFA] and under this condition for 0.01 min). Purity: 86.99%, Rt=2.24 min; MS Calcd.: 393.2; MS Found: 394.1 [M+H]⁺.

### 7.8 the Synthesis of 7-11

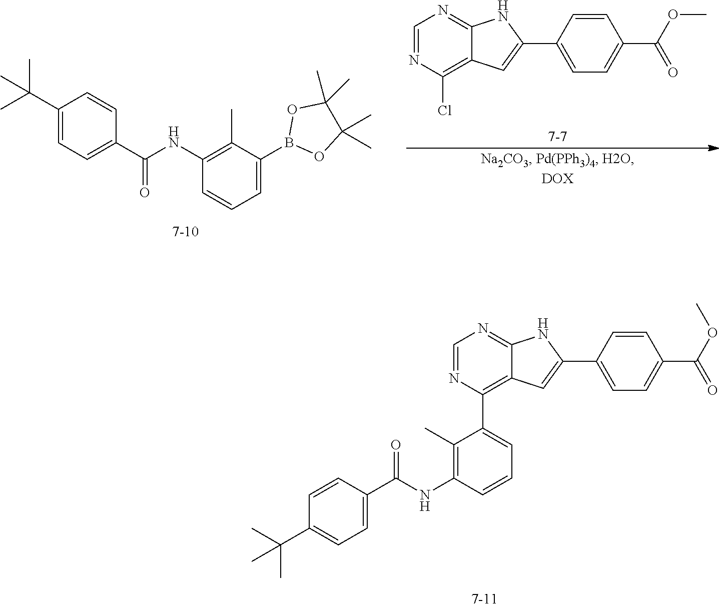 [SMILES: Cc1c(NC(=O)c2ccc(C(C)(C)C)cc2)cccc1B1OC(C)(C)C(C)(C)O1 ; COC(=O)c1ccc(-c2cc3c(Cl)ncnc3[nH]2)cc1 ; COC(=O)c1ccc(-c2cc3c(-c4cccc(NC(=O)c5ccc(C(C)(C)C)cc5)c4C)ncnc3[nH]2)cc1]

A solution of 4-tert-butyl-N-(2-methyl-3-(4,4,5,5-tetramethyl-1,3,2-dioxaborolan-2-yl)phenyl)benzamide (7-1, 1.37 g, 3.48 mmol), methyl 4-(4-chloro-7H-pyrrolo[2,3-d]pyrimidin-6-yl)benzoate (1.2 g, 3.48 mmol), K₂CO₃ (1.44 g, 10.44 mmol), Pd(PPh₃)₄ (402 mg, 0.35 mmol) in DOX (12 mL) and water (3 mL) was heated in the microwave at 120° C. for 1 h. After cooling to room temperature the mixture was concentrated to give the residue, the crude material was purified by flash column chromatography to give 7-11 (1.5 g, 83% yield) as a purple solid. LCMS (Agilent LCMS 1200-6110, Column: Waters X-Bridge C18 (50 mm*4.6 mm*3.5 μm); Column Temperature: 40° C.; Flow Rate: 1.5 mL/min; Mobile Phase: from 95% [water+0.05% TFA] and 5% [CH₃CN+0.05% TFA] to 0% [water+0.05% TFA] and 100% [CH₃CN+0.05% TFA] in 0.8 min, then under this condition for 0.4 min, finally changed to 95% [water+0.05% TFA] and 5% [CH₃CN+0.05% TFA] and under this condition for 0.01 min). Purity: 88.47%, Rt=1.83 min; MS Calcd.: 518.2; MS Found: 519.2 [M+H]⁺.

### 7.9 the Synthesis of 7-12

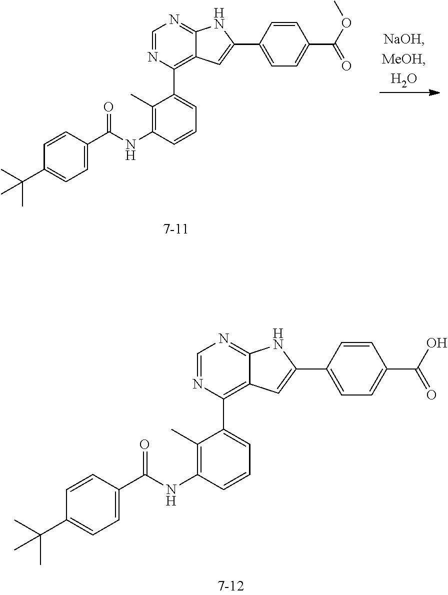 [SMILES: COC(=O)c1ccc(-c2cc3c(-c4cccc(NC(=O)c5ccc(C(C)(C)C)cc5)c4C)ncnc3[nH]2)cc1 ; Cc1c(NC(=O)c2ccc(C(C)(C)C)cc2)cccc1-c1ncnc2[nH]c(-c3ccc(C(=O)O)cc3)cc12]

To a solution of methyl 4-(4-(3-(4-tert-butylbenzamido)-2-methylphenyl)-7H-pyrrolo[2,3-d]pyrimidin-6-yl)benzoate (7-11, 1.6 g, 3.0 mmol) in MeOH/H₂O (10/10 mL) was added NaOH (240 mg, 6.0 mmol), then the mixture was stirred at 80° C. overnight. After completion of the reaction indicated by LCMS, the mixture was quenched with water and adjusted to pH=6 by Citric acid solution and filtered, the solid was washed with H₂O and concentrated to 7-12 (500 mg, 33.3%) as brown solid. LCMS (Agilent LCMS 1200-6110, Column: Waters X-Bridge C18 (50 mm*4.6 mm*3.5 μm); Column Temperature: 40° C.; Flow Rate: 1.5 mL/min; Mobile Phase: from 95% [water+0.05% TFA] and 5% [CH₃CN+0.05% TFA] to 0% [water+0.05% TFA] and 100% [CH₃CN+0.05% TFA] in 0.8 min, then under this condition for 0.4 min, finally changed to 95% [water+0.05% TFA] and 5% [CH₃CN+0.05% TFA] and under this condition for 0.01 min). Purity: 100.00%, Rt=1.329 min; MS Calcd.: 504.2; MS Found: 449.3 [M+H]⁺. HPLC (Agilent HPLC 1200, Column: L-column2 ODS (150 mm*4.6 mm*5.0 μm); Column Temperature: 40° C.; Flow Rate: 1.0 mL/min; Mobile Phase: from 90% [(total 10 mM AcONH₄) H₂O/MeCN=900/100 (v/v)] and 10% [total 10 mM AcONH₄) H₂O/MeCN=100/900 (v/v)] to 15% [total 10 mM AcONH₄) H₂O/MeCN=900/100 (v/v)] and 85% [total 10 mM AcONH₄) H₂O/MeCN=100/900 (v/v)] in 5 min, then under this condition for 10 min, finally changed to 90% [(total 10 mM AcONH₄) H₂O/MeCN=900/100 (v/v)] and 10% [total 10 mM AcONH₄) H₂O/MeCN=100/900 (v/v)] in 0.1 min and under this condition for 5 min. Purity: 100.00%, Rt=5.796 min; MS Calcd.: 504.2; MS Found: 449.2 [M+H]⁺. ¹H NMR (400 MHZ, DMSO-d₆)δ: 12.89 (s, 1H), 10.05 (s, 1H), 8.89 (s, 1H), 8.09 (d, J=8.4 Hz, 2H), 8.02 (q, J=2.0 Hz, 4H), 7.63-7.60 (m, 1H), 7.57-7.54 (m, 3H), 7.48-7.41 (m, 2H), 6.96 (s, 1H), 2.21 (s, 3H).

### 7.10 the Synthesis of Compound I-7

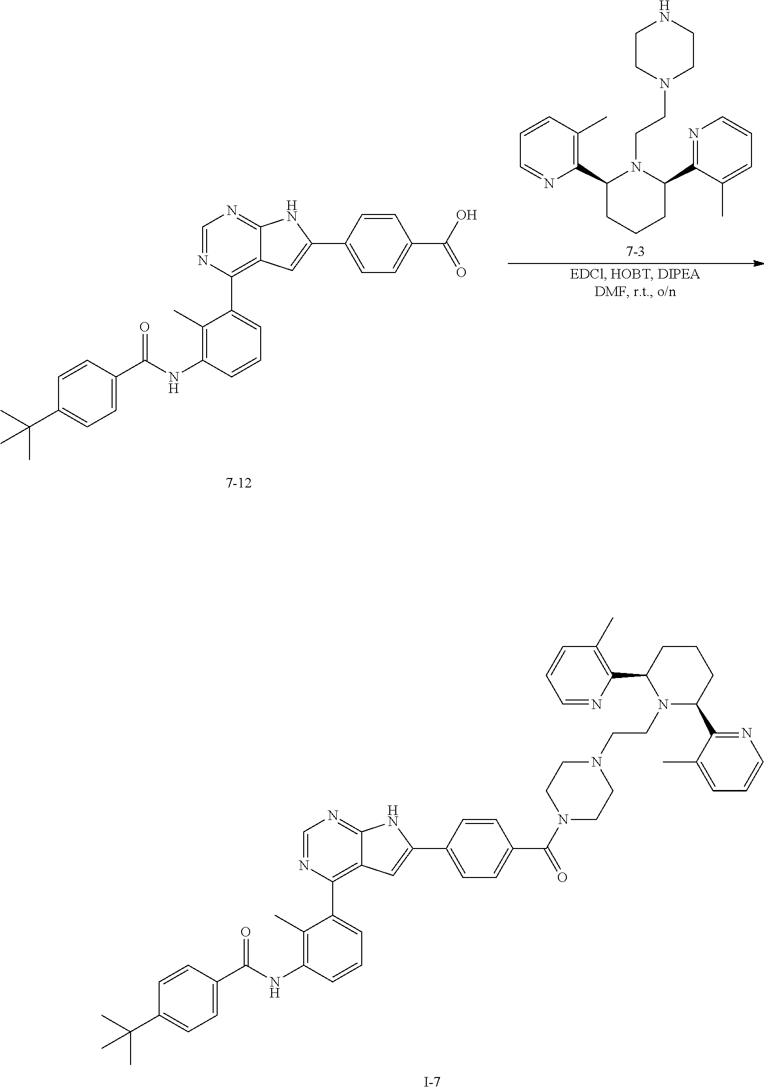

A solution of 4-(4-(3-(4-tert-butylbenzamido)-2-methylphenyl)-7H-pyrrolo[2,3-d]pyrimidin-6-yl)benzoic acid (7-12, 93 mg, 0.185 mmol), 1-(2-((2S,6R)-2,6-bis(3-methylpyridin-2-yl)piperidin-1-yl)ethyl)piperazine (7-3, 70 mg, 0.185 mmol), EDCI (53 mg, 0.277 mmol), DIPEA (71 mg, 0.554 mmol) and HOBT (37 mg, 0.277 mmol) in DMF (4 mL) was stirred at room temperature for overnight. After completion of the reaction indicated by LCMS, the mixture was concentrated in vacuum to give the residue, which was purified by prep-HPLC to give I-7 (28.02 mg, 18%) as white solid. LCMS (Agilent LCMS 1200-6120, Column: Waters X-Bridge C18 (50 mm*4.6 mm*3.5 μm); Column Temperature: 40° C.; Flow Rate: 2.0 mL/min; Mobile Phase: from 95% [water+10 mM NH₄HCO₃] and 5% [CH₃CN] to 0% [water+10 mM NH₄HCO₃] and 100% [CH₃CN] in 1.6 min, then under this condition for 1.4 min, finally changed to 95% [water+10 mM NH₄HCO₃] and 5% [CH₃CN] in 0.1 min and under this condition for 0.7 min.) Purity: 100.00%. Rt=2.025 min; MS Calcd.: 865.5; MS Found: 866.7 [M+H]⁺. HPLC (Agilent HPLC 1200, Column: L-column2 ODS (150 mm*4.6 mm*5.0 μm); Column Temperature: 40° C.; Flow Rate: 1.0 mL/min; Mobile Phase: from 90% [(total 10 mM AcONH₄) H₂O/MeCN=900/100 (v/v)] and 10% [total 10 mM AcONH₄) H₂O/MeCN=100/900 (v/v)] to 15% [total 10 mM AcONH₄) H₂O/MeCN=900/100 (v/v)] and 85% [total 10 mM AcONH₄) H₂O/MeCN=100/900 (v/v)] in 5 min, then under this condition for 10 min, finally changed to 90% [(total 10 mM AcONH₄) H₂O/MeCN=900/100 (v/v)] and 10% [total 10 mM AcONH₄) H₂O/MeCN=100/900 (v/v)] in 0.1 min and under this condition for 5 min. Purity: 99.50%, Rt=10.360 min; MS Calcd.: 865.5; MS Found: 866.6 [M+H]⁺. ¹H NMR (400 MHZ, DMSO-d₆): 12.81 (s, 1H), 9.95 (s, 1H), 8.87 (s, 1H), 8.35 (d, J=4.0 Hz, 2H), 7.98 (q, J=4.0 Hz, 4H), 7.55 (t, J=8.4 Hz, 5H), 7.46-7.39 (m, 2H), 7.35 (d, J=8.4 Hz, 2H), 7.18 (q, J=2.4 Hz, 2H), 6.87 (s, 1H), 4.28 (d, J=10.4 Hz, 2H), 3.29-3.22 (m, 2H), 3.02 (s, 2H), 2.54 (s, 7H), 2.41-2.33 (m, 2H), 2.23 (s, 5H), 2.01-1.99 (m, 1H), 1.70-1.67 (m, 1H), 1.54-1.45 (m, 5H), 1.33 (s, 9H), 0.93 (s, 2H).

### Example 8: Synthesis of Compound I-8

### Synthetic Scheme of Compound I-8

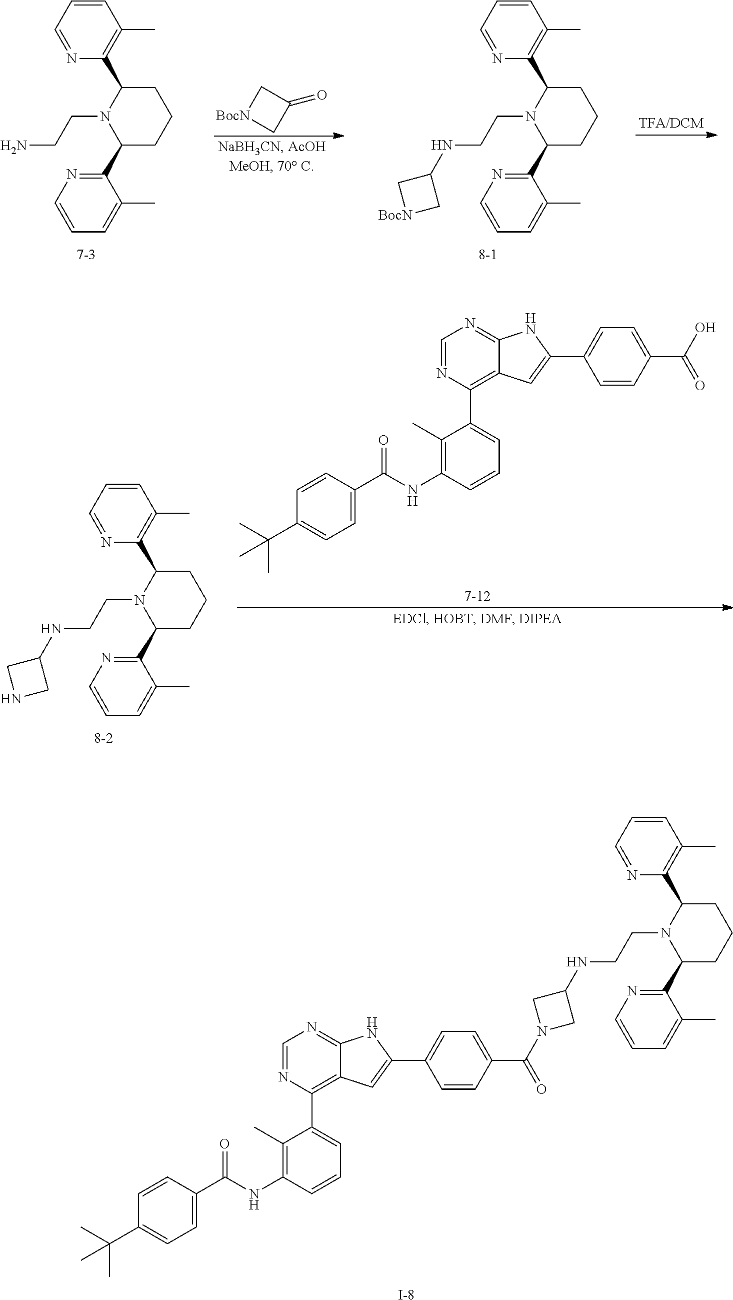

### 8.1 the Synthesis of 8-1

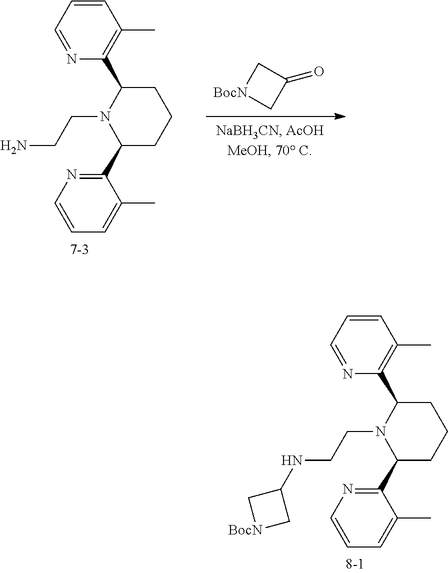 [SMILES: Cc1cccnc1[C@H]1CCC[C@@H](c2ncccc2C)N1CCN ; CC(C)(C)OC(=O)N1CC(=O)C1 ; Cc1cccnc1[C@H]1CCC[C@@H](c2ncccc2C)N1CCNC1CN(C(=O)OC(C)(C)C)C1]

To a solution of 2-((2R,6S)-2,6-bis(3-methylpyridin-2-yl)piperidin-1-yl) ethanamine (7-3, 500 mg, 1.613 mmol), tert-butyl 3-oxoazetidine-1-carboxylate (644 mg, 4.839 mmol) and AcOH (107 mg, 1.774 mmol) in CH₃OH (15 mL) was added NaBH₃CN (204 mg, 3.226 mmol), then the mixture was stirred at room temperature overnight. After completion of the reaction indicated by TLC, the reaction mixture was alkalixed to pH=8 with NaHCO₃ aqueous solution and concentrated to give the residue, which was purified by column chromatography (DCM/CH₃OH=100/5) to give tert-butyl 3-(2-((2R,6S)-2,6-bis(3-methylpyridin-2-yl)piperidin-1-yl)ethylamino) azetidine-1-carboxylate (8-1, 240 mg, 32%) as a colorless liquid. LCMS (Agilent LCMS 1200-6120, Column: Waters X-Bridge C18 (50 mm*4.6 mm*3.5 μm); Column Temperature: 40° C.; Flow Rate: 2.0 mL/min; Mobile Phase: from 95% [water+10 mM NH₄HCO₃] and 5% [CH₃CN] to 0% [water+10 mM NH₄HCO₃] and 100% [CH₃CN] in 1.6 min, then under this condition for 1.4 min, finally changed to 95% [water+10 mM NH₄HCO₃] and 5% [CH₃CN] in 0.1 min and under this condition for 0.7 min.) Purity: 73%. Rt=1.874 min; MS Calcd.: 465.3; MS Found: 466.4 [M+H]⁺.

### 8.2 the Synthesis of 8-2

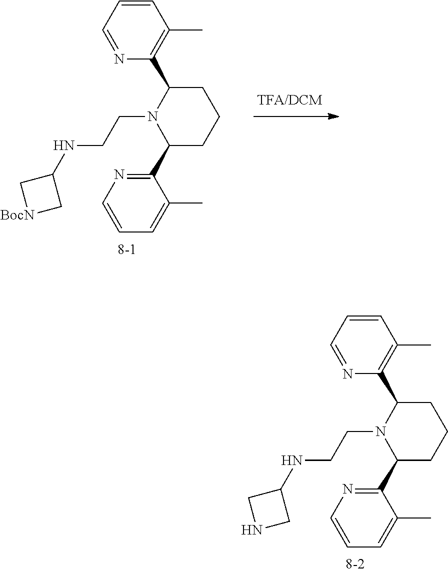 [SMILES: Cc1cccnc1[C@H]1CCC[C@@H](c2ncccc2C)N1CCNC1CN(C(=O)OC(C)(C)C)C1 ; Cc1cccnc1[C@H]1CCC[C@@H](c2ncccc2C)N1CCNC1CNC1]

To a solution of 3-(2-((2R, 6S)-2, 6-bis(3-methylpyridin-2-yl)piperidin-1-yl)ethylamino) azetidine-1-carboxylate (8-1, 240 mg, 0.516 mmol) in dry DCM (8 mL) was added TFA (2 mL), then the mixture was stirred at room temperature overnight. After completion of the reaction indicated by TLC, the mixture was concentrated in vacuum, diluted with water, adjusted to pH=9 by NaOH solution and extracted with DCM/MeOH=30:1 (15 mL×3). The organic layer was washed with brine, dried over Na₂SO₄, filtered and concentrated in vacuum to give the residue, which was purified by column chromatography (DCM/MeOH=20:1) to give N-(2-((2R,6S)-2,6-bis(3-methylpyridin-2-yl)piperidin-1-yl)ethyl) azetidin-3-amine (8-2, 160 mg, 85%) as colorless liquid.

LCMS (Agilent LCMS 1200-6120, Column: Waters X-Bridge C18 (50 mm*4.6 mm*3.5 μm); Column Temperature: 40° C.; Flow Rate: 2.0 mL/min; Mobile Phase: from 95% [water+10 mM NH₄HCO₃] and 5% [CH₃CN] to 0% [water+10 mM NH₄HCO₃] and 100% [CH₃CN] in 1.6 min, then under this condition for 1.4 min, finally changed to 95% [water+10 mM NH₄HCO₃] and 5% [CH₃CN] in 0.1 min and under this condition for 0.7 min.) Purity: 100%. Rt=1.531 min; MS Calcd.: 365.3; MS Found: 366.4 [M+H]⁺.

### 8.3 the Synthesis of Compound I-8

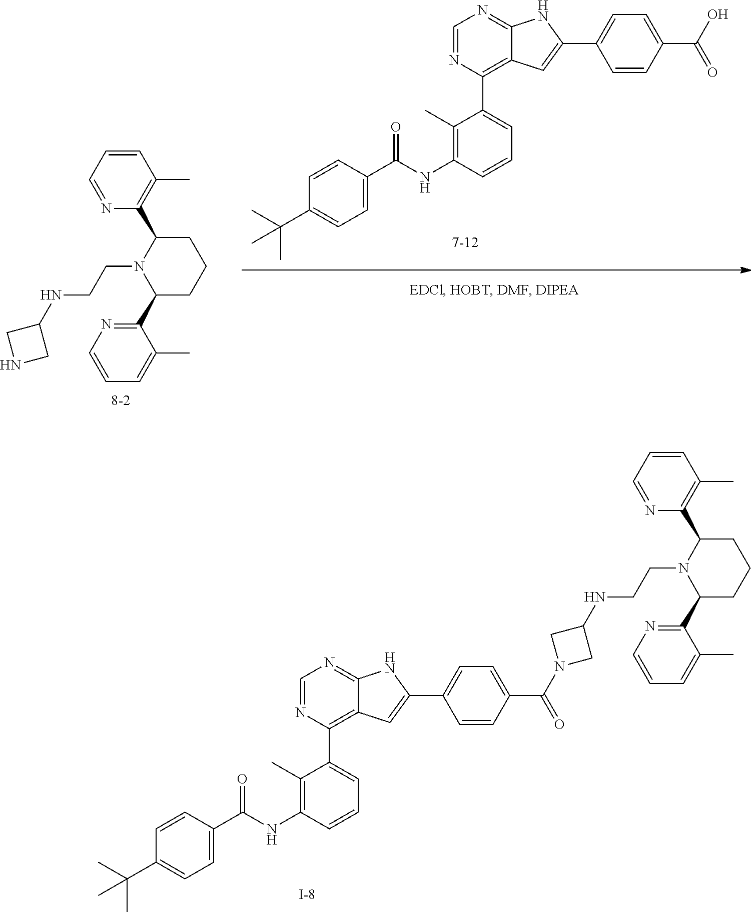

To a solution of N-(2-((2R,6S)-2,6-bis(3-methylpyridin-2-yl)piperidin-1-yl)ethyl) azetidin-3-amine (8-2, 50 mg, 0.137 mmol), 4-(4-(3-(4-tert-butylbenzamido)-2-methylphenyl)-7H-pyrrolo[2,3-d]pyrimidin-6-yl)benzoic acid (7-12, 73 mg, 0.144 mmol), EDCI (40 mg, 0.206 mmol) and HOBT (30 mg, 0.206 mmol) in DMF (5 mL) was added DIPEA (60 mg, 0.412 mmol), then the mixture was stirred at room temperature overnight. After completion of the reaction indicated by TLC, then concentrated to give the residue, which was purified by column chromatography and prep-HPLC to give I-8 (9 mg, 8%) as a yellow solid.

LCMS (Agilent LCMS 1200-6120, Column: Waters X-Bridge C18 (50 mm*4.6 mm*3.5 μm); Column Temperature: 40° C.; Flow Rate: 2.0 mL/min; Mobile Phase: from 95% [water+10 mM NH₄HCO₃] and 5% [CH₃CN] to 0% [water+10 mM NH₄HCO₃] and 100% [CH₃CN] in 1.6 min, then under this condition for 1.4 min, finally changed to 95% [water+10 mM NH₄HCO₃] and 5% [CH₃CN] in 0.1 min and under this condition for 0.7 min.) Purity: 100%. Rt=1.979 min; MS Calcd.: 851.5; MS Found: 852.6 [M+H]⁺. HPLC (Agilent HPLC 1200, Column: L-column2 ODS (150 mm*4.6 mm*5.0 μm); Column Temperature: 40° C.; Flow Rate: 1.0 mL/min; Mobile Phase: from 90% [(total 10 mM AcONH₄) H₂O/MeCN=900/100 (v/v)] and 10% [total 10 mM AcONH₄) H₂O/MeCN=100/900 (v/v)] to 15% [total 10 mM AcONH₄) H₂O/MeCN=900/100 (v/v)] and 85% [total 10 mM AcONH₄) H₂O/MeCN=100/900 (v/v)] in 5 min, then under this condition for 10 min, finally changed to 90% [(total 10 mM AcONH₄) H₂O/MeCN=900/100 (v/v)] and 10% [total 10 mM AcONH₄) H₂O/MeCN=100/900 (v/v)] in 0.1 min and under this condition for 5 min. Purity: 100.00%, Rt=10.04 min; MS Calcd.: 851.5; MS Found: 852.6 [M+H]⁺. ¹H NMR (400 MHZ, DMSO-d₆)δ: 12.86 (s, 1H), 9.95 (s, 1H), 8.87 (s, 1H), 8.35 (s, 2H), 8.07-8.05 (d, J=8.4 Hz, 2H), 7.97-7.95 (d, J=8.4 Hz, 2H), 7.61-7.48 (m, 8H), 7.46-7.40 (m, 2H), 7.18-7.15 (m, 2H), 6.92 (s, 1H), 4.26-4.23 (m, 2H), 4.02-3.98 (m, 2H), 3.78-3.74 (m, 2H), 3.44-3.41 (m, 1H), 3.27-3.19 (m, 1H), 2.68 (s, 6H), 2.33-2.28 (m, 2H), 2.26-2.07 (m, 5H), 2.00-1.97 (m, 1H), 1.69-1.67 (m, 1H), 1.48-1.46 (d, J=11.2 Hz, 2H), 1.33 (s, 10H), 1.23-1.14 (m, 2H).

### Example 9: Synthesis of Compound I-9

### Synthetic Scheme of Compound I-9

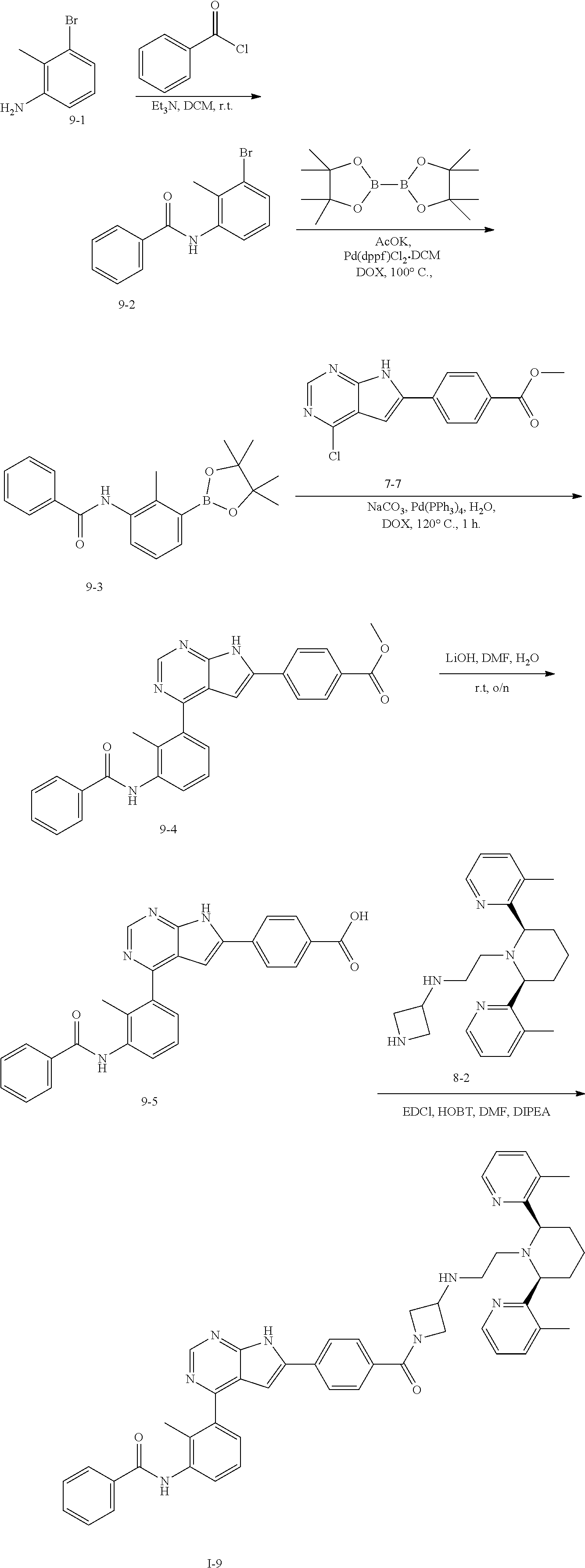

### 9.1 the Synthesis of 9-2

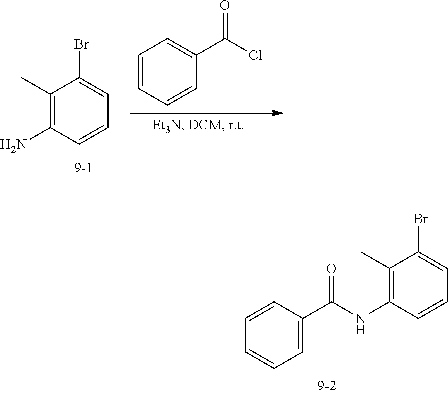

To a solution of 3-bromo-2-methylaniline (9-1, 3.0 g, 16.22 mmol), benzoyl chloride (2.3 g, 16.22 mmol) in DCM (40 mL) was added triethylamine (3.3 g, 32.44 mmol) at room temperature for overnight. After completion of the reaction indicated by LCMS, the mixture was quenched with water, extracted with dichloromethane (20 mL×3). The combined organic layers were washed with brine, dried over anhydrous Na₂SO₄, filtered and concentrated in vacuo to give the residue, which was purified by column chromatography to give N-(3-bromo-2-methylphenyl)benzamide (9-2, 2.5 g, 53%) as light yellow solid. LCMS (Agilent LCMS 1200-6120, Column: Waters X-Bridge C18 (50 mm*4.6 mm*3.5 μm); Column Temperature: 40° C.; Flow Rate: 2.0 mL/min; Mobile Phase: from 95% [water+10 mM NH₄HCO₃] and 5% [CH₃CN] to 0% [water+10 mM NH₄HCO₃] and 100% [CH₃CN] in 1.6 min, then under this condition for 1.4 min, finally changed to 95% [water+10 mM NH₄HCO₃] and 5% [CH₃CN] in 0.1 min and under this condition for 0.7 min.) Purity: 89.89%. Rt=1.648 min; MS Calcd.: 289.0; MS Found: 290.0 [M+H]⁺.

### 9.2 the Synthesis of 9-3

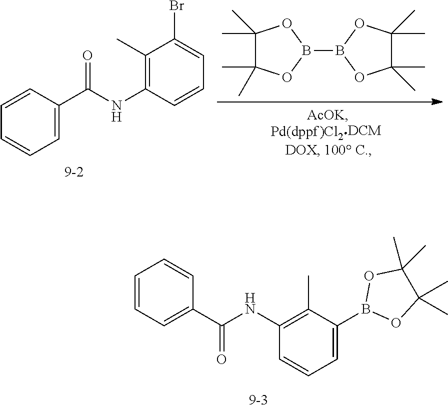

A solution of N-(3-bromo-2-methylphenyl)benzamide (9-2, 2.0 g, 6.92 mmol), 4,4,4′,4′,5,5,5′,5′-octamethyl-2,2′-bi (1,3,2-dioxaborolane (1.93 g, 7.62 mmol), AcOK (2.03 g, 20.76 mmol), and Pd(dppf)Cl₂·DCM (559 mg, 0.69 mmol) in DOX (30 mL) under N₂ at room temperature was stirred at 100° C. for 16 h. After cooling to room temperature the mixture was diluted with EA and washed with water. The organic phase was dried over Na₂SO₄, filtered and concentrated. The crude material was purified by flash column chromatography on silica gel (PE:EA=10:1) to give N-(2-methyl-3-(4, 4, 5, 5-tetramethyl-1,3,2-dioxaborolan-2-yl)phenyl)benzamide (9-3, 1.8 g, 78% yield) as a white solid.

LCMS (Agilent LCMS 1200-6110, Column: Waters X-Bridge C18 (50 mm*4.6 mm*3.5 μm); Column Temperature: 40° C.; Flow Rate: 1.5 mL/min; Mobile Phase: from 95% [water+0.05% TFA] and 5% [CH₃CN+0.05% TFA] to 0% [water+0.05% TFA] and 100% [CH₃CN+0.05% TFA] in 0.8 min, then under this condition for 0.4 min, finally changed to 95% [water+0.05% TFA] and 5% [CH₃CN+0.05% TFA] and under this condition for 0.01 min). Purity: 89.70%, Rt=2.007 min; MS Calcd.: 337.2; MS Found: 338.1 [M+H]⁺.

### 9.3 the Synthesis of 9-4

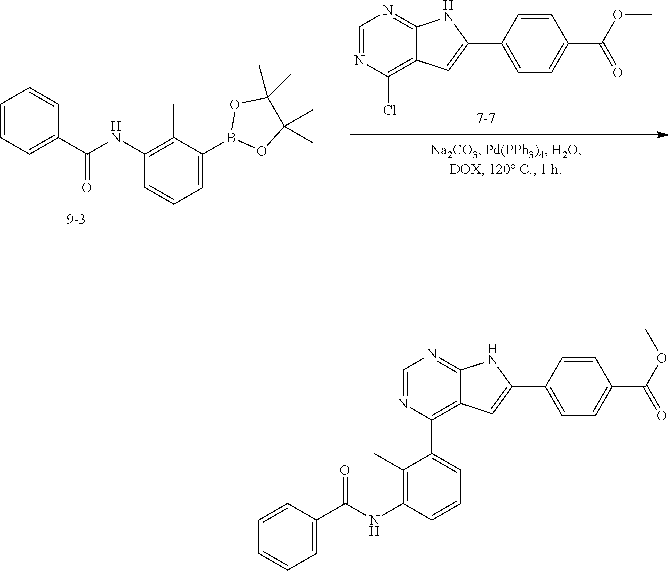 [SMILES: Cc1c(NC(=O)c2ccccc2)cccc1B1OC(C)(C)C(C)(C)O1 ; COC(=O)c1ccc(-c2cc3c(Cl)ncnc3[nH]2)cc1 ; COC(=O)c1ccc(-c2cc3c(-c4cccc(NC(=O)c5ccccc5)c4C)ncnc3[nH]2)cc1]

A solution of N-(2-methyl-3-(4,4,5,5-tetramethyl-1,3,2-dioxaborolan-2-yl)phenyl)benzamide (9-3, 1.0 g, 3.48 mmol), methyl 4-(4-chloro-7H-pyrrolo[2,3-d]pyrimidin-6-yl)benzoate (7-7, 1.2 g, 3.48 mmol), K₂CO₃ (1.44 g, 10.44 mmol), Pd(PPh₃)₄ (402 mg, 0.35 mmol) in DOX (12 mL) and water (3 mL) was heated in the microwave at 120° C. for 1 h. After cooling to room temperature the mixture was concentrated to give the residue, the crude material was purified by flash column chromatography to give methyl4-(4-(3-benzamido-2-methylphenyl)-7H-pyrrolo[2,3-d]pyrimidin-6-yl)benzoate (9-4, 1.1 g, 80% yield) as a purple solid. LCMS (Agilent LCMS 1200-6110, Column: Waters X-Bridge C18 (50 mm*4.6 mm*3.5 μm); Column Temperature: 40° C.; Flow Rate: 1.5 mL/min; Mobile Phase: from 95% [water+0.05% TFA] and 5% [CH₃CN+0.05% TFA] to 0% [water+0.05% TFA] and 100% [CH₃CN+0.05% TFA] in 0.8 min, then under this condition for 0.4 min, finally changed to 95% [water+0.05% TFA] and 5% [CH₃CN+0.05% TFA] and under this condition for 0.01 min). Purity: 84.23%, Rt=1.584 min; MS Calcd.: 462.2; MS Found: 463.2 [M+H]⁺.

### 9.4 the Synthesis of 9-5

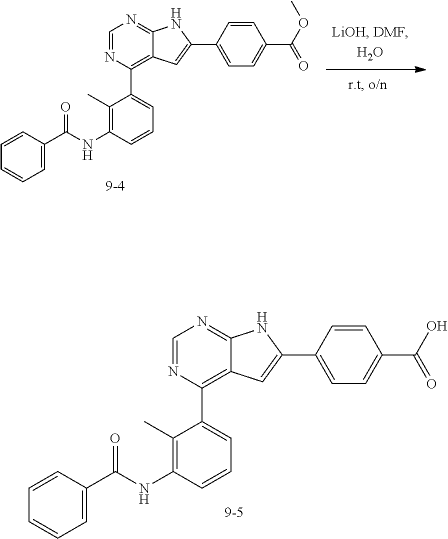 [SMILES: COC(=O)c1ccc(-c2cc3c(-c4cccc(NC(=O)c5ccccc5)c4C)ncnc3[nH]2)cc1 ; Cc1c(NC(=O)c2ccccc2)cccc1-c1ncnc2[nH]c(-c3ccc(C(=O)O)cc3)cc12]

To a solution of methyl 4-(4-(3-benzamido-2-methylphenyl)-7H-pyrrolo[2,3-d]pyrimidin-6-yl)benzoate (9-4, 1.0 g, 2.16 mmol) in DMF (20 mL) was added LiOH (363 mg, 8.64 mmol, in H₂O (5 mL) at room temperature overnight. After completion of the reaction indicated by LCMS, the mixture was quenched with water and adjusted to pH=6 by Citric acid solution and filtered, which was purified by column chromatography and prep-HPLC to give 4-(4-(3-benzamido-2-methylphenyl)-7H-pyrrolo[2, 3-d]pyrimidin-6-yl)benzoic acid (9-5, 600 mg, 61.8%) as a light yellow solid. LCMS (Agilent LCMS 1200-6110, Column: Waters X-Bridge C18 (50 mm*4.6 mm*3.5 μm); Column Temperature: 40° C.; Flow Rate: 1.5 mL/min; Mobile Phase: from 95% [water+0.05% TFA] and 5% [CH₃CN+0.05% TFA] to 0% [water+0.05% TFA] and 100% [CH₃CN+0.05% TFA] in 0.8 min, then under this condition for 0.4 min, finally changed to 95% [water+0.05% TFA] and 5% [CH₃CN+0.05% TFA] and under this condition for 0.01 min). Purity: 100.00%, Rt=1.329 min; MS Calcd.: 448.2; MS Found: 449.3 [M+H]⁺. HPLC (Agilent HPLC 1200, Column: L-column2 ODS (150 mm*4.6 mm*5.0 μm); Column Temperature: 40° C.; Flow Rate: 1.0 mL/min; Mobile Phase: from 90% [(total 10 mM AcONH₄) H₂O/MeCN=900/100 (v/v)] and 10% [total 10 mM AcONH₄) H₂O/MeCN=100/900 (v/v)] to 15% [total 10 mM AcONH₄) H₂O/MeCN=900/100 (v/v)] and 85% [total 10 mM AcONH₄) H₂O/MeCN=100/900 (v/v)] in 5 min, then under this condition for 10 min, finally changed to 90% [(total 10 mM AcONH₄) H₂O/MeCN=900/100 (v/v)] and 10% [total 10 mM AcONH₄) H₂O/MeCN=100/900 (v/v)] in 0.1 min and under this condition for 5 min. Purity: 100.00%, Rt=5.796 min; MS Calcd.: 448.2; MS Found: 449.2 [M+H]⁺.

¹H NMR (400 MHZ, DMSO-d₆)δ: 12.89 (s, 1H), 10.05 (s, 1H), 8.89 (s, 1H), 8.09 (d, J=8.4 Hz, 2H), 8.02 (q, J=2.0 Hz, 4H), 7.63-7.60 (m, 1H), 7.57-7.54 (m, 3H), 7.48-7.41 (m, 2H), 6.96 (s, 1H), 2.21 (s, 3H).

### 9.5 the Synthesis of Compound I-9

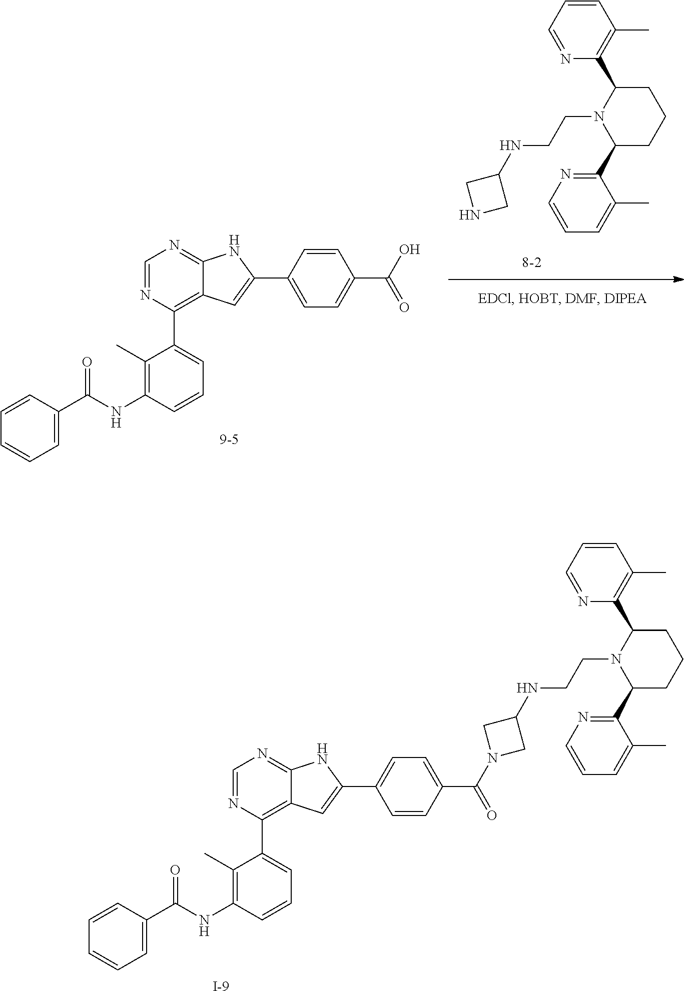

To a solution of N-(2-((2R,6S)-2,6-bis(3-methylpyridin-2-yl)piperidin-1-yl)ethyl) azetidin-3-amine (8-2, 50 mg, 0.137 mmol), 4-(4-(3-(4-tert-butylbenzamido)-2-methylphenyl)-7H-pyrrolo[2,3-d]pyrimidin-6-yl)benzoic acid (9-5, 62 mg, 0.137 mmol), EDCI (40 mg, 0.206 mmol) and HOBT (30 mg, 0.206 mmol) in DMF (5 mL) was added DIPEA (60 mg, 0.412 mmol), then the mixture was stirred at room temperature overnight. After completion of the reaction indicated by TLC, then concentrated to give the residue, which was purified by column chromatography and prep-HPLC to give I-9 (12 mg, 11%) as a yellow solid.

LCMS (Agilent LCMS 1200-6120, Column: Waters X-Bridge C18 (50 mm*4.6 mm*3.5 μm); Column Temperature: 40° C.; Flow Rate: 2.0 mL/min; Mobile Phase: from 95% [water+10 mM NH₄HCO₃] and 5% [CH₃CN] to 0% [water+10 mM NH₄HCO₃] and 100% [CH₃CN] in 1.6 min, then under this condition for 1.4 min, finally changed to 95% [water+10 mM NH₄HCO₃] and 5% [CH₃CN] in 0.1 min and under this condition for 0.7 min.) Purity: 100%. Rt=1.768 min; MS Calcd.: 795.4; MS Found: 796.5 [M+H]⁺.

HPLC (Agilent HPLC 1200, Column: L-column2 ODS (150 mm*4.6 mm*5.0 μm); Column Temperature: 40° C.; Flow Rate: 1.0 mL/min; Mobile Phase: from 90% [(total 10 mM AcONH₄) H₂O/MeCN=900/100 (v/v)] and 10% [total 10 mM AcONH₄) H₂O/MeCN=100/900 (v/v)] to 15% [total 10 mM AcONH₄) H₂O/MeCN=900/100 (v/v)] and 85% [total 10 mM AcONH₄) H₂O/MeCN=100/900 (v/v)] in 5 min, then under this condition for 10 min, finally changed to 90% [(total 10 mM AcONH₄) H₂O/MeCN=900/100 (v/v)] and 10% [total 10 mM AcONH₄) H₂O/MeCN=100/900 (v/v)] in 0.1 min and under this condition for 5 min. Purity: 100.00%, Rt=8.688 min; MS Calcd.: 795.4; MS Found: 796.4 [M+H]⁺.

¹H NMR (400 MHZ, DMSO-d₆)δ: 12.86 (s, 1H), 10.05 (s, 1H), 8.88 (s, 1H), 8.35 (s, 2H), 8.07-8.02 (d, J=22.8 Hz, 4H), 7.63-7.49 (m, 8H), 7.48-7.41 (m, 3H), 7.18-7.15 (m, 2H), 6.93 (s, 1H), 4.26-4.23 (m, 2H), 4.00-3.98 (m, 1H), 3.78-3.74 (m, 1H), 3.44-3.41 (m, 1H), 3.26-3.18 (m, 1H), 2.67 (s, 6H), 2.38-2.33 (m, 2H), 2.21-2.08 (m, 5H), 2.01-1.97 (m, 1H), 1.69-1.64 (m, 1H), 1.49-1.46 (d, J=11.6 Hz, 2H), 1.37-1.35 (m, 1H), 1.23-1.13 (m, 2H).

### Example 10: Synthesis of Compound I-10

### 10.1 Synthesis of Compound I-10

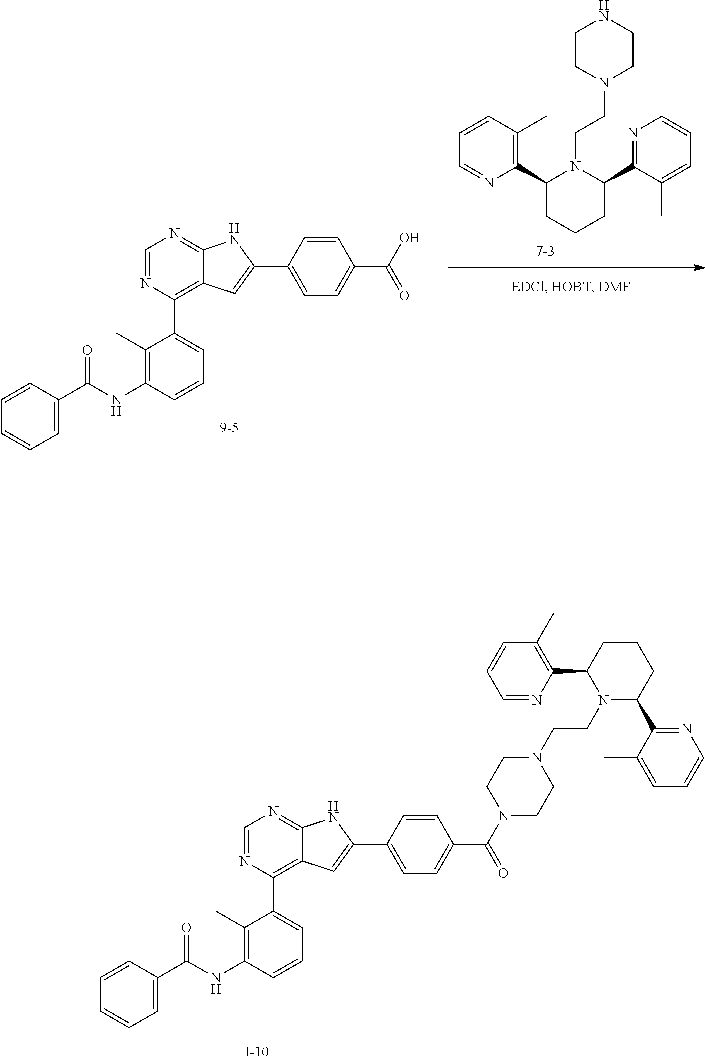

A solution of 1-(2-((2S,6R)-2,6-bis(3-methylpyridin-2-yl)piperidin-1-yl)ethyl)piperazine (7-3, 60 mg, 0.16 mmol), 4-(4-(3-benzamido-2-methylphenyl)-7H-pyrrolo[2, 3-d]pyrimidin-6-yl)benzoic acid (9-5, 71 mg, 0.16 mmol), EDCI (46 mg, 0.24 mmol), DIPEA (62 mg, 0.48 mmol) in DMF (5 mL) was stirred at room temperature for 20 min, followed by adding HOBT (33 mg, 0.24 mmol). The reaction mixture was allowed to stir at room temperature overnight. After completion of the reaction indicated by LCMS, the mixture was filtered to give the filtrate, which was purified by column chromatography and prep-HPLC to give N-(3-(6-(4-(4-(2-((2R,6S)-2,6-bis(3-methylpyridin-2-yl)piperidin-1-yl)ethyl)piperazine-1-carbonyl)phenyl)-7H-pyrrolo[2,3-d]pyrimidin-4-yl)-2-methylphenyl)benzamide (I-10, 34.68 mg, 27%) as white solid. LCMS (Agilent LCMS 1200-6120, Column: Waters X-Bridge C18 (50 mm*4.6 mm*3.5 μm); Column Temperature: 40° C.; Flow Rate: 2.0 mL/min; Mobile Phase: from 95% [water+10 mM NH₄HCO₃] and 5% [CH₃CN] to 0% [water+10 mM NH₄HCO₃] and 100% [CH₃CN] in 1.6 min, then under this condition for 1.4 min, finally changed to 95% [water+10 mM NH₄HCO₃] and 5% [CH₃CN] in 0.1 min and under this condition for 0.7 min.) Purity: 100.00%. Rt=1.821 min; MS Calcd.: 809.4; MS Found: 810.5 [M+H]⁺. HPLC (Agilent HPLC 1200, Column: L-column2 ODS (150 mm*4.6 mm*5.0 μm); Column Temperature: 40° C.; Flow Rate: 1.0 mL/min; Mobile Phase: from 90% [(total 10 mM AcONH₄) H₂O/MeCN=900/100 (v/v)] and 10% [total 10 mM AcONH₄) H₂O/MeCN=100/900 (v/v)] to 15% [total 10 mM AcONH₄) H₂O/MeCN=900/100 (v/v)] and 85% [total 10 mM AcONH₄) H₂O/MeCN=100/900 (v/v)] in 5 min, then under this condition for 10 min, finally changed to 90% [(total 10 mM AcONH₄) H₂O/MeCN=900/100 (v/v)] and 10% [total 10 mM AcONH₄) H₂O/MeCN=100/900 (v/v)] in 0.1 min and under this condition for 5 min. Purity: 100.00%, Rt=9.024 min; MS Calcd.: 809.4; MS Found: 810.4 [M+H]⁺.

¹H NMR (400 MHZ, DMSO-d₆)δ: 10.06 (s, 1H), 8.86 (s, 1H), 8.33 (d, J=2.8 Hz, 2H), 8.00 (q, J=7.6 Hz, 4H), 7.61 (t, J=6.8 Hz, 1H), 7.54 (d, J=7.6 Hz, 5H), 7.42 (d, J=7.2 Hz, 2H), 7.34 (d, J=8.0 Hz, 2H), 7.16 (d, J=4.8 Hz, 2H), 6.86 (s, 1H), 4.26 (d, J=10.0 Hz, 2H), 3.68 (s, 1H), 3.27 (s, 1H), 3.02 (s, 2H), 2.52 (s, 8H), 3.39 (s, 1H), 2.19-2.06 (m, 5H), 1.97 (s, 1H), 1.68-1.45 (m, 7H), 1.07-0.97 (m, 1H).

### Example 11: Synthesis of Compound I-11

### Synthetic Scheme of Compound I-11

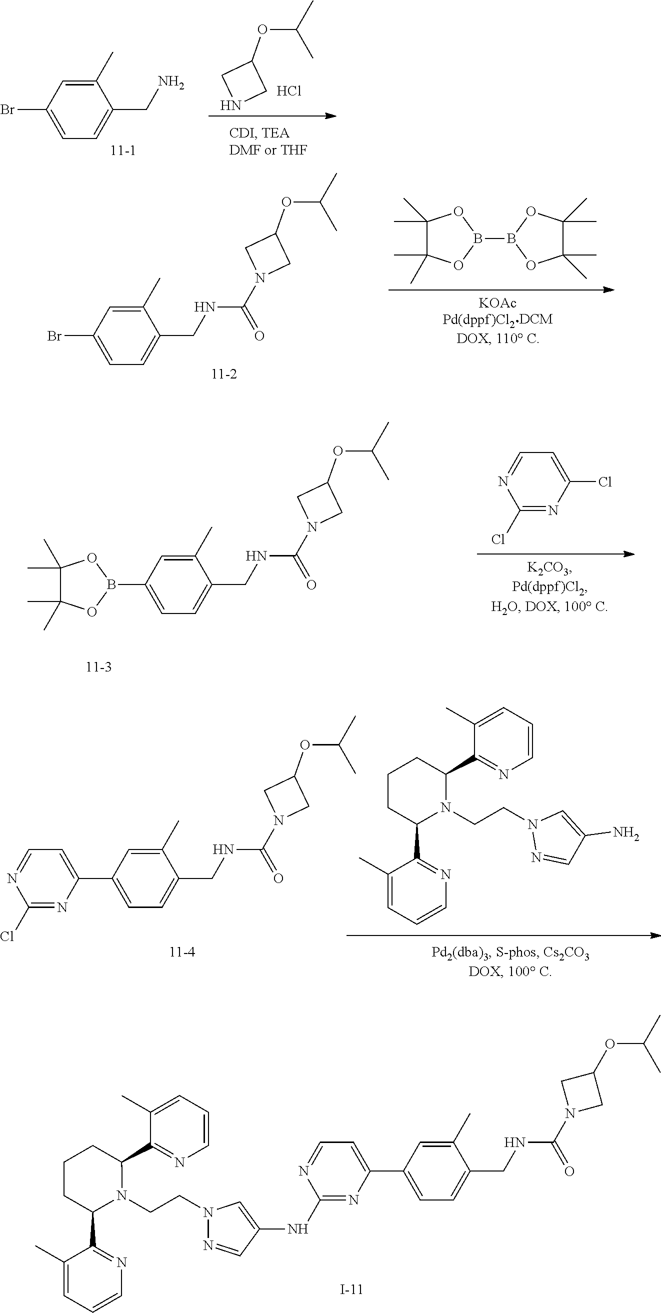

### 11.1 the Synthesis of 11-2

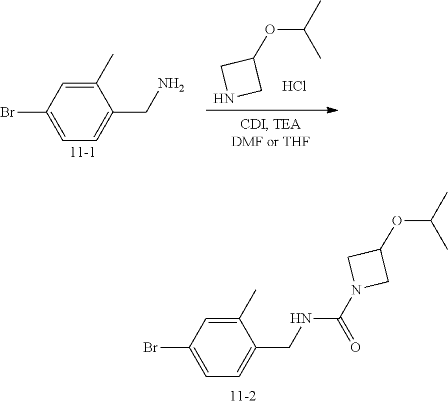

To a solution of (4-bromo-2-methylphenyl) methanamine (11-1, 500 mg, 2.499 mmol) in THF (150 mL) was added CDI (608 mg, 3.749 mmol) and TEA (379 mg, 3.749 mmol), then the mixture was stirred at room temperature for 1 h, 3-isopropoxyazetidine hydrochloride was added to the solution, the mixture was stirred at room temperature for 1 h. After completion of the reaction indicated by LCMS, the mixture was concentrated in vacuum to give a residue, which was purified by column chromatography (PE:EA=2:1) to give the desired product (11-2, 680 mg, 80%) as white solid.

### 11.2 the Synthesis of 11-3

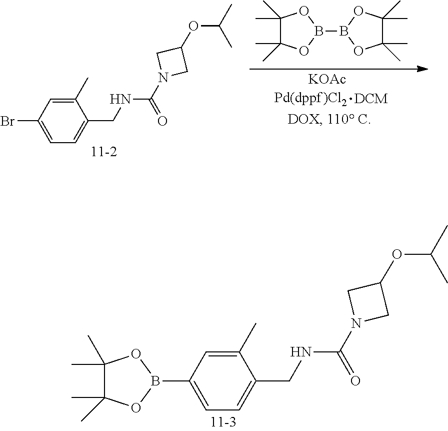

To a solution of N-(4-bromo-2-methylbenzyl)-3-isopropoxyazetidine-1-carboxamide (11-2, 680 mg, 1.999 mmol) in DOX (15 mL) was added 4,4,4′,4′,5,5,5′,5′-octamethyl-2,2′-bi (1,3,2-dioxaborolane) (558 mg, 2.199 mmol), KOAc (392 mg, 3.999 mmol) and Pd(dppf)Cl₂·DCM (163 mg, 0.200 mmol), then the mixture was stirred at 110° C. for overnight. After completion of the reaction indicated by LCMS, the mixture was filtered and concentrated in vacuum to give a residue, which was purified by column chromatography (PE:EA=2:1) to give the desired product (11-3, 450 mg, 58%) as yellow oil.

### 11.3 the Synthesis of 11-4

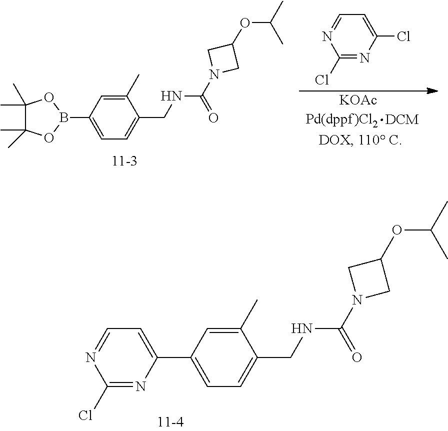 [SMILES: Cc1cc(B2OC(C)(C)C(C)(C)O2)ccc1CNC(=O)N1CC(OC(C)C)C1 ; Clc1ccnc(Cl)n1 ; Cc1cc(-c2ccnc(Cl)n2)ccc1CNC(=O)N1CC(OC(C)C)C1 ; "KOAc"]

To a solution of 3-isopropoxy-N-(2-methyl-4-(4,4,5,5-tetramethyl-1,3,2-dioxaborolan-2-yl)benzyl) azetidine-1-carboxamide (11-3, 450 mg, 1.159 mmol) in DOX/H₂O (20 mL/4 mL) was added 2,4-dichloropyrimidine (190 mg, 1.275 mmol), K₂CO₃ (320 mg, 2.318 mmol) and Pd(dppf)Cl₂ (42 mg, 0.058 mmol), then the mixture was stirred at 100° C. for 2 h. After completion of the reaction indicated by LCMS, the mixture was filtered and poured into water (15 mL) and extracted with DCM (30 mL×3). The organic layer was dried over Na₂SO₄, filtered and concentrated in vacuum to give a residue, which was purified by column chromatography (DCM:MeOH=20:1) to give the desired product (11-4, 450 mg, 92%) as yellow solid.

### 11.4 the Synthesis of Compound I-11

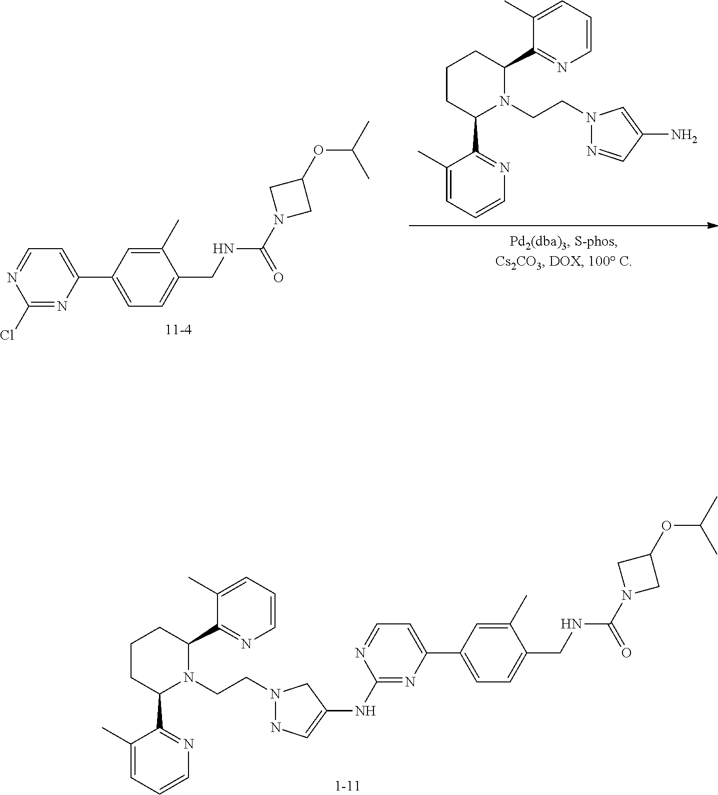 [SMILES: Cc1cc(-c2ccnc(Cl)n2)ccc1CNC(=O)N1CC(OC(C)C)C1 ; Cc1cccnc1[C@H]1CCC[C@@H](c2ncccc2C)N1CCn1cc(N)cn1 ; Cc1cc(-c2ccnc(NC3=CNN(CCN4[C@@H](c5ncccc5C)CCC[C@H]4c4ncccc4C)C3)n2)ccc1CNC(=O)N1CC(OC(C)C)C1]

To a solution of N-(4-(2-chloropyrimidin-4-yl)-2-methylbenzyl)-3-isopropoxyazetidine-1-carboxamide (11-4, 120 mg, 0.321 mmol) in DOX (10 mL) was added 1-(2-((2R,6S)-2,6-bis(3-methylpyridin-2-yl)piperidin-1-yl)ethyl)-1H-pyrazol-4-amine (110 mg, 0.292 mmol), Cs₂CO₃ (285 mg, 0.875 mmol), S-Phos (26 mg, 0.064 mmol) and Pd₂(dba)₃ (27 mg, 0.029 mmol), then the mixture was stirred at 100° C. for overnight. After completion of the reaction indicated by LCMS, the mixture was filtered and concentrated in vacuum to give a residue, which was purified by prep-HPLC to give the desired product I-11 (29.28 mg, 14%) as yellow solid.

LCMS (Agilent LCMS 1200-6120, Column: Waters X-Bridge C18 (50 mm*4.6 mm*3.5 μm); Column Temperature: 40° C.; Flow Rate: 2.0 mL/min; Mobile Phase: from 95% [water+10 mM NH₄HCO₃] and 5% [CH₃CN] to 0% [water+10 mM NH₄HCO₃] and 100% [CH₃CN] in 1.6 min, then under this condition for 1.4 min, finally changed to 95% [water+10 mM NH₄HCO₃] and 5% [CH₃CN] in 0.1 min and under this condition for 0.7 min.) Purity: 100.00%. Rt=2.043 min; MS Calcd.: 714.4; MS Found: 715.0 [M+H]⁺.

HPLC (Agilent HPLC 1200, Column: L-column2 ODS (150 mm*4.6 mm*5.0 μm); Column Temperature: 40° C.; Flow Rate: 1.0 mL/min; Mobile Phase: from 90% [(total 10 mM AcONH₄) H₂O/MeCN=900/100 (v/v)] and 10% [total 10 mM AcONH₄) H₂O/MeCN=100/900 (v/v)] to 15% [total 10 mM AcONH₄) H₂O/MeCN=900/100 (v/v)] and 85% [total 10 mM AcONH₄) H₂O/MeCN=100/900 (v/v)] in 5 min, then under this condition for 10 min, finally changed to 90% [(total 10 mM AcONH₄) H₂O/MeCN=900/100 (v/v)] and 10% [total 10 mM AcONH₄) H₂O/MeCN=100/900 (v/v)] in 0.1 min and under this condition for 5 min. Purity: 100.00%, Rt=9.174 min; MS Calcd.: 714.4; MS Found: 715.0 [M+H]⁺.

¹H NMR (400 MHZ, DMSO-d₆) &: 9.31 (s, 1H), 8.46 (s, 1H), 8.36 (s, 2H), 7.89 (d, J=8.0 Hz, 2H), 7.52 (s, 2H), 7.40 (s, 1H), 7.24-7.16 (m, 4H), 6.99 (s, 1H), 6.88 (s, 1H), 4.36 (d, J=10.8 Hz, 2H), 4.32-4.28 (m, 1H), 4.25 (d, J=5.2 Hz, 2H), 4.04 (q, J=6.8 Hz, 2H), 3.62 (dd, J=4.4, 8.8 Hz, 2H), 3.56 (q, J=6.4 Hz, 1H), 2.73 (s, 2H), 2.68-2.51 (m, 8H), 2.38 (s, 3H), 2.21 (d, J=10.8 Hz, 2H), 2.01 (d, J=11.6 Hz, 1H), 1.71 (q, J=10.4 Hz, 1H), 1.49 (d, J=12.4 Hz, 2H), 1.07 (d, J=6.4 Hz, 6H).

### Example 12: Synthesis of Compound I-12

### Synthetic Scheme of Compound I-12

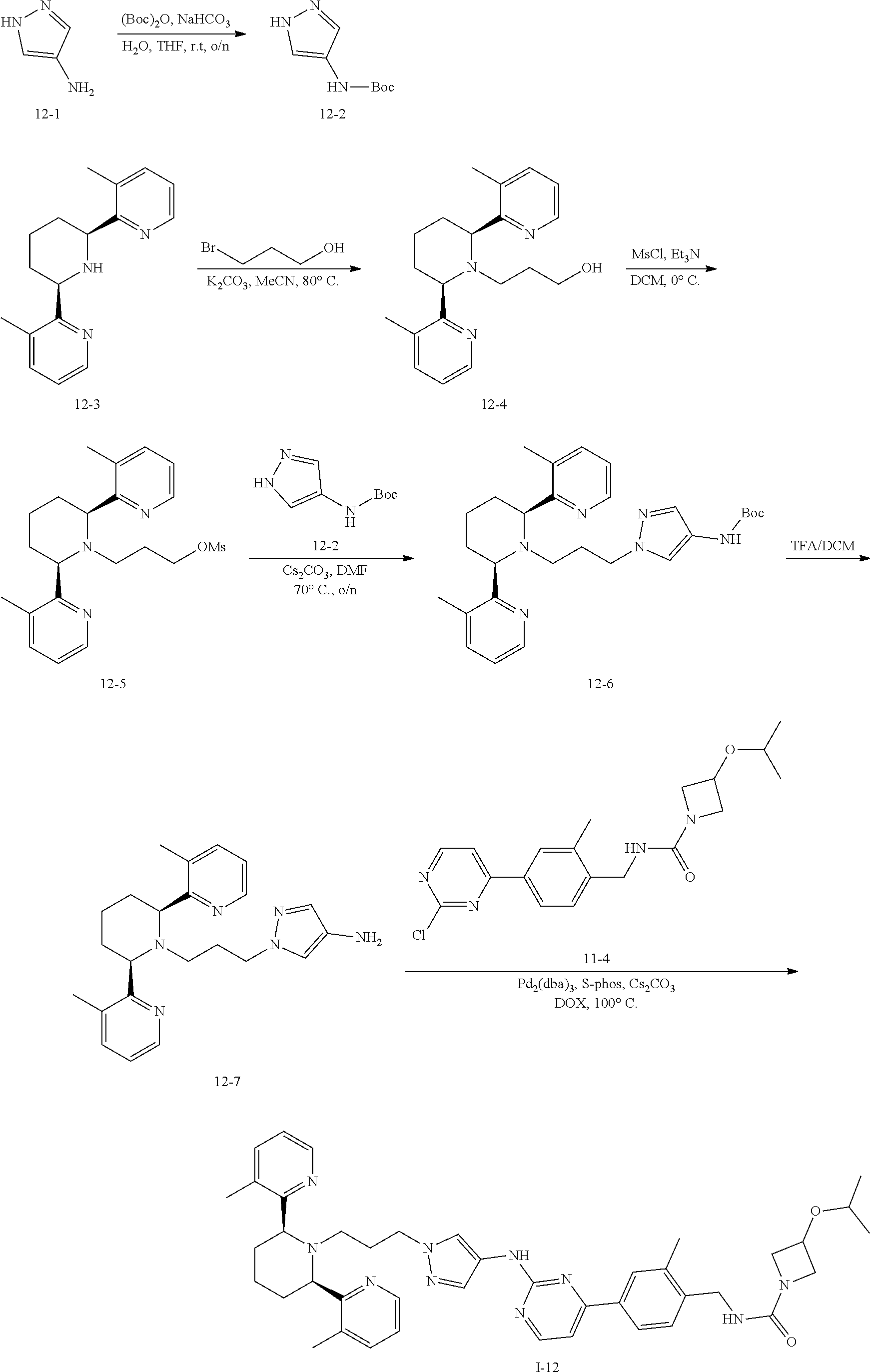

### 12.1 the Synthesis of 12-2

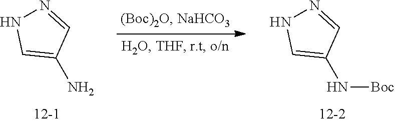 [SMILES: Nc1cn[nH]c1 ; CC(C)(C)OC(=O)Nc1cn[nH]c1]

To a solution of 1H-pyrazol-4-amine (12-1, 1.0 g, 12.05 mmol) in dry THF (16 mL) was slowly added di-tert-butyl dicarbonate (5.2 g, 24.0 mmol) at room temperature, followed by adding a solution of NaHCO₃ (2.0 g, 24.0 mmol) in water (10 mL) at room temperature, then the mixture was stirred at room temperature overnight. After completion of the reaction indicated by LCMS, the reaction mixture was added water (20 mL), and extracted with dichloromethane (20 mL×3). The combined organic layers were washed with brine, dried over anhydrous Na₂SO₄, filtered and concentrated in vacuo to give the residue, which was purified by column chromatography to give tert-butyl 1H-pyrazol-4-ylcarbamate (12-2, 1.2 g, 55%) as light yellow syrup. LCMS (Agilent LCMS 1200-6120, Column: Waters X-Bridge C18 (50 mm*4.6 mm*3.5 μm); Column Temperature: 40° C.; Flow Rate: 2.0 mL/min; Mobile Phase: from 95% [water+10 mM NH₄HCO₃] and 5% [CH₃CN] to 0% [water+10 mM NH₄HCO₃] and 100% [CH₃CN] in 1.6 min, then under this condition for 1.4 min, finally changed to 95% [water+10 mM NH₄HCO₃] and 5% [CH₃CN] in 0.1 min and under this condition for 0.7 min.) Purity: 93.91%. Rt=1.359 min; MS Calcd.: 183.1; MS Found: 184.2 [M+H]⁺.

### 12.2 the Synthesis of 12-4

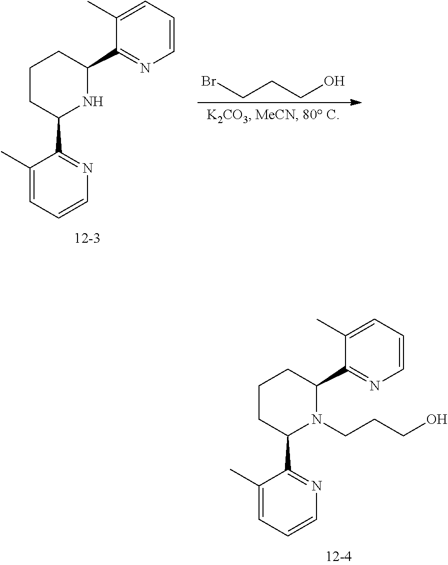 [SMILES: Cc1cccnc1[C@@H]1CCC[C@H](c2ncccc2C)N1 ; OCCCBr ; Cc1cccnc1[C@H]1CCC[C@@H](c2ncccc2C)N1CCCO]

To a solution of (2R, 6S)-2, 6-bis(3-methylpyridin-2-yl)piperidine (12-3, 600 mg, 2.247 mmol) and 3-bromopropan-1-ol (465 mg, 3.371 mmol) in MeOH (20 mL) was added K₂CO₃ (620 mg, 4.494 mmol), then the mixture was stirred at 80° C. overnight. After completion of the reaction indicated by LCMS, the reaction mixture was concentrated to give the residue, which was purified by column chromatography to give 3-((2R,6S)-2,6-bis(3-methylpyridin-2-yl)piperidin-1-yl) propan-1-ol (12-4, 656 mg, 90%) as white solid.

LCMS (Agilent LCMS 1200-6120, Column: Waters X-Bridge C18 (50 mm*4.6 mm*3.5 μm); Column Temperature: 40° C.; Flow Rate: 2.0 mL/min; Mobile Phase: from 95% [water+10 mM NH₄HCO₃] and 5% [CH₃CN] to 0% [water+10 mM NH₄HCO₃] and 100% [CH₃CN] in 1.6 min, then under this condition for 1.4 min, finally changed to 95% [water+10 mM NH₄HCO₃] and 5% [CH₃CN] in 0.1 min and under this condition for 0.7 min.) Purity: 94.08%. Rt=1.616 min; MS Calcd.: 325.2; MS Found: 326.2 [M+H]⁺.

### 12.3 the Synthesis of 12-5

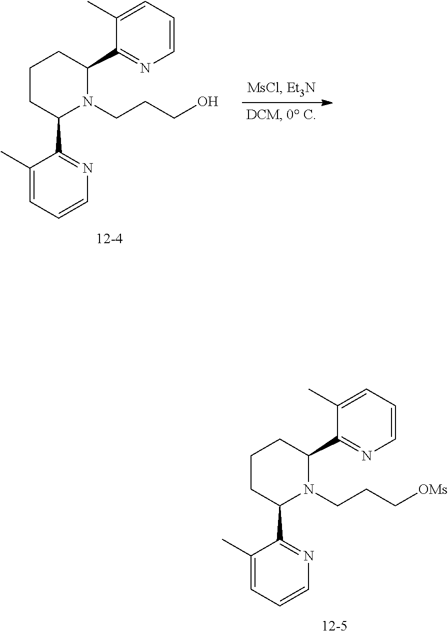 [SMILES: Cc1cccnc1[C@H]1CCC[C@@H](c2ncccc2C)N1CCCO ; Cc1cccnc1[C@H]1CCC[C@@H](c2ncccc2C)N1CCCOS(C)(=O)=O]

To a solution of 3-((2R, 6S)-2, 6-bis(3-methylpyridin-2-yl)piperidin-1-yl) propan-1-ol (12-4, 400 mg, 1.230 mmol) and MsCl (220 mg, 1.846 mmol) in DCM (20 mL) was added Et₃N (260 mg, 2.460 mmol), then the mixture was stirred at 0° C. for 20 min. After completion of the reaction indicated by LCMS, the reaction mixture was concentrated to give the residue, which was purified by column chromatography to give 3-((2R,6S)-2,6-bis(3-methylpyridin-2-yl)piperidin-1-yl) propyl methanesulfonate (12-5, 480 mg, 96%) as colorless liquid. LCMS (Agilent LCMS 1200-6120, Column: Waters X-Bridge C18 (50 mm*4.6 mm*3.5 μm); Column Temperature: 40° C.; Flow Rate: 2.0 mL/min; Mobile Phase: from 95% [water+10 mM NH₄HCO₃] and 5% [CH₃CN] to 0% [water+10 mM NH₄HCO₃] and 100% [CH₃CN] in 1.6 min, then under this condition for 1.4 min, finally changed to 95% [water+10 mM NH₄HCO₃] and 5% [CH₃CN] in 0.1 min and under this condition for 0.7 min.) Purity: 80.99%. Rt=1.769 min; MS Calcd.: 403.2; MS Found: 404.0 [M+H]. 12.4 The synthesis of 12-6

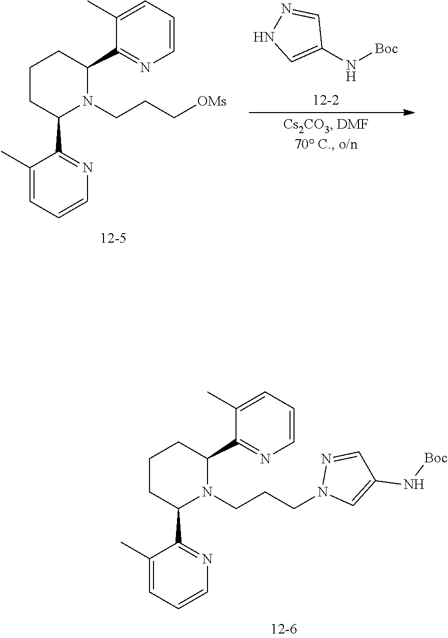 [SMILES: Cc1cccnc1[C@H]1CCC[C@@H](c2ncccc2C)N1CCCOS(C)(=O)=O ; CC(C)(C)OC(=O)Nc1cn[nH]c1 ; Cc1cccnc1[C@H]1CCC[C@@H](c2ncccc2C)N1CCCn1cc(NC(=O)OC(C)(C)C)cn1]

To a solution of 3-((2R,6S)-2,6-bis(3-methylpyridin-2-yl)piperidin-1-yl) propyl methanesulfonate (12-5, 226 mg, 0.571 mmol) and tert-butyl 1-(3-((2R,6S)-2,6-bis(3-methylpyridin-2-yl)piperidin-1-yl) propyl)-1H-pyrazol-4-ylcarbamate (12-2, 99 mg, 0.544 mmol) in DMF (8 mL) was added Cs₂CO₃ (532 mg, 1.632 mmol), then the mixture was stirred at 70° C. overnight. After completion of the reaction indicated by LCMS, the reaction mixture was concentrated to give the residue, which was purified by column chromatography (DCM/MeOH=20:1) to give tert-butyl 1-(3-((2R,6S)-2,6-bis(3-methylpyridin-2-yl)piperidin-1-yl) propyl)-1H-pyrazol-4-ylcarbamate (12-6, 187 mg, 70%) as colorless solid.

LCMS (Agilent LCMS 1200-6120, Column: Waters X-Bridge C18 (50 mm*4.6 mm*3.5 μm); Column Temperature: 40° C.; Flow Rate: 2.0 mL/min; Mobile Phase: from 95% [water+10 mM NH₄HCO₃] and 5% [CH₃CN] to 0% [water+10 mM NH₄HCO₃] and 100% [CH₃CN] in 1.6 min, then under this condition for 1.4 min, finally changed to 95% [water+10 mM NH₄HCO₃] and 5% [CH₃CN] in 0.1 min and under this condition for 0.7 min.) Purity: 86%. Rt=1.885 min; MS Calcd.: 490.3; MS Found: 491.1 [M+H]⁺.

### 12.5 the Synthesis of 12-7

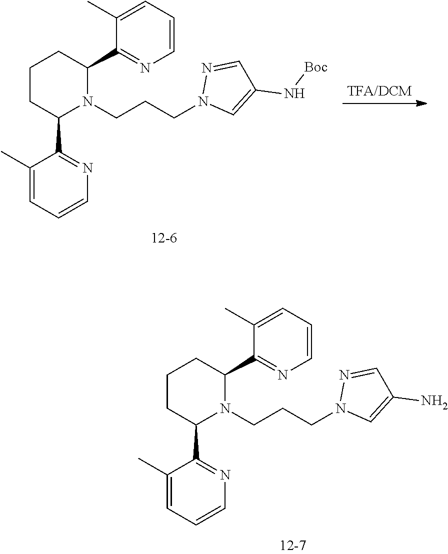 [SMILES: Cc1cccnc1[C@H]1CCC[C@@H](c2ncccc2C)N1CCCn1cc(NC(=O)OC(C)(C)C)cn1 ; Cc1cccnc1[C@H]1CCC[C@@H](c2ncccc2C)N1CCCn1cc(N)cn1]

To a solution of tert-butyl 1-(3-((2R, 6S)-2,6-bis(3-methylpyridin-2-yl)piperidin-1-yl) propyl)-1H-pyrazol-4-ylcarbamate (12-6, 187 mg, 0.382 mmol) in dry DCM (8 mL) was added TFA (2 mL), then the mixture was stirred at room temperature overnight. After completion of the reaction indicated by TLC, the mixture was concentrated in vacuum, diluted with water, adjusted to pH=9 by NaOH solution and extracted with DCM/MeOH=30:1 (15 mL×3). The organic layer was washed with brine, dried over Na₂SO₄, filtered and concentrated in vacuum to give the residue, which was purified by column chromatography (DCM/MeOH=20:1) to give 1-(3-((2R,6S)-2,6-bis(3-methylpyridin-2-yl)piperidin-1-yl) propyl)-1H-pyrazol-4-amine (12-7, 56 mg, 38%) as colorless solid. LCMS (Agilent LCMS 1200-6120, Column: Waters X-Bridge C18 (50 mm*4.6 mm*3.5 μm); Column Temperature: 40° C.; Flow Rate: 2.0 mL/min; Mobile Phase: from 95% [water+10 mM NH₄HCO₃] and 5% [CH₃CN] to 0% [water+10 mM NH₄HCO₃] and 100% [CH₃CN] in 1.6 min, then under this condition for 1.4 min, finally changed to 95% [water+10 mM NH₄HCO₃] and 5% [CH₃CN] in 0.1 min and under this condition for 0.7 min.) Purity: 86%. Rt=1.566 min; MS Calcd.: 390.3; MS Found: 391.1 [M+H]⁺.

### 12-6 the Synthesis of Compound I-12

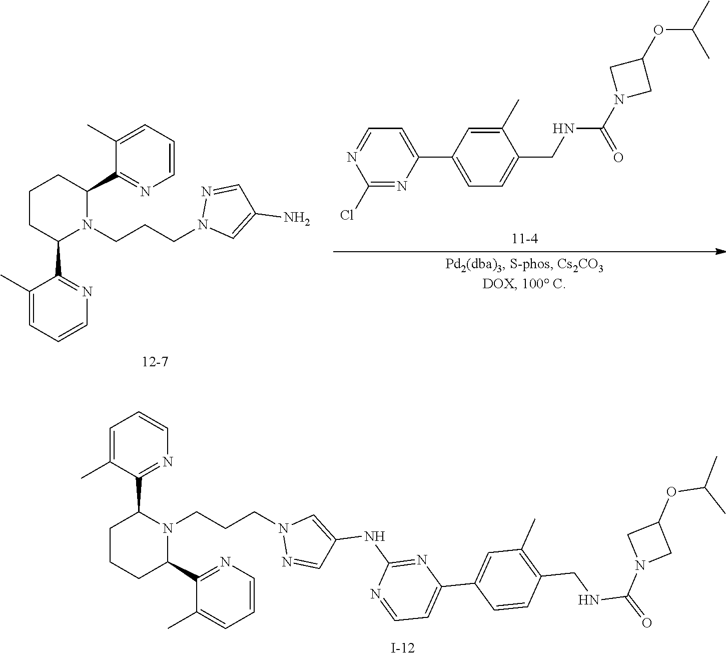

To a solution of N-(4-(2-chloropyrimidin-4-yl)-2-methylbenzyl)-3-isopropoxyazetidine-1-carboxamide (11-4, 58 mg, 0.155 mmol) in DOX (8 mL) was added 1-(3-((2R,6S)-2,6-bis(3-methylpyridin-2-yl)piperidin-1-yl) propyl)-1H-pyrazol-4-amine (12-7, 55 mg, 0.141 mmol), Cs₂CO₃ (138 mg, 0.0.423 mmol), S-Phos (12 mg, 0.0282 mmol) and Pd₂(dba)₃ (13 mg, 0.0141 mmol), then the mixture was stirred at 100° C. for overnight. After completion of the reaction indicated by LCMS, the mixture was filtered and concentrated in vacuum to give a residue, which was purified by column chromatography and prep-HPLC to give the desired product N-(4-(2-(1-(3-((2S,6R)-2,6-bis(3-methylpyridin-2-yl)piperidin-1-yl) propyl)-1H-pyrazol-4-ylamino)pyrimidin-4-yl)-2-methylbenzyl)-3-isopropoxyazetidine-1-carboxamide (I-12, 14 mg, 14%) as yellow solid. LCMS (Agilent LCMS 1200-6120, Column: Waters X-Bridge C18 (50 mm*4.6 mm*3.5 μm); Column Temperature: 40° C.; Flow Rate: 2.0 mL/min; Mobile Phase: from 95% [water+10 mM NH₄HCO₃] and 5% [CH₃CN] to 0% [water+10 mM NH₄HCO₃] and 100% [CH₃CN] in 1.6 min, then under this condition for 1.4 min, finally changed to 95% [water+10 mM NH₄HCO₃] and 5% [CH₃CN] in 0.1 min and under this condition for 0.7 min.) Purity: 100.00%. Rt=1.82 min; MS Calcd.: 728.4; MS Found: 729.1 [M+H]⁺. HPLC (Agilent HPLC 1200, Column: L-column2 ODS (150 mm*4.6 mm*5.0 μm); Column Temperature: 40° C.; Flow Rate: 1.0 mL/min; Mobile Phase: from 90% [(total 10 mM AcONH₄) H₂O/MeCN=900/100 (v/v)] and 10% [total 10 mM AcONH₄) H₂O/MeCN=100/900 (v/v)] to 15% [total 10 mM AcONH₄) H₂O/MeCN=900/100 (v/v)] and 85% [total 10 mM AcONH₄) H₂O/MeCN=100/900 (v/v)] in 5 min, then under this condition for 10 min, finally changed to 90% [(total 10 mM AcONH₄) H₂O/MeCN=900/100 (v/v)] and 10% [total 10 mM AcONH₄) H₂O/MeCN=100/900 (v/v)] in 0.1 min and under this condition for 5 min. Purity: 100.00%, Rt=9.01 min; MS Calcd.: 728.4; MS Found: 729.4 [M+H]⁺. ¹H NMR (400 MHZ, DMSO-d₆)δ: 9.35 (s, 1H), 7.47-7.46 (d, J=4 Hz, 2H), 8.29 (s, 2H), 7.91-7.89 (d, J=8 Hz, 2H), 7.46 (s, 2H), 7.38-7.36 (d, J=8 Hz, 2H), 7.30 (s, 1H), 7.26-7.24 (d, J=8 Hz, 2H), 7.05 (s, 2H), 7.47-7.46 (t, J=4.8 Hz, 2H), 4.35-4.25 (m, 1H), 4.26-4.20 (m, 4H), 4.07-4.03 (m, 2H), 3.65-3.62 (m, 2H), 3.59-3.55 (m, 1H), 3.10 (s, 1H), 2.53 (s, 6H), 2.37-2.33 (m, 5H), 2.13-2.08 (m, 2H), 1.94 (s, 1H), 1.66-1.64 (m, 1H), 1.49-1.44 (m, 2H), 1.08 (s, 3H), 1.07 (s, 3H), 0.68-0.65 (m, 2H).

### Example 13: Synthesis of Compound I-13

### Synthetic Scheme of Compound I-13

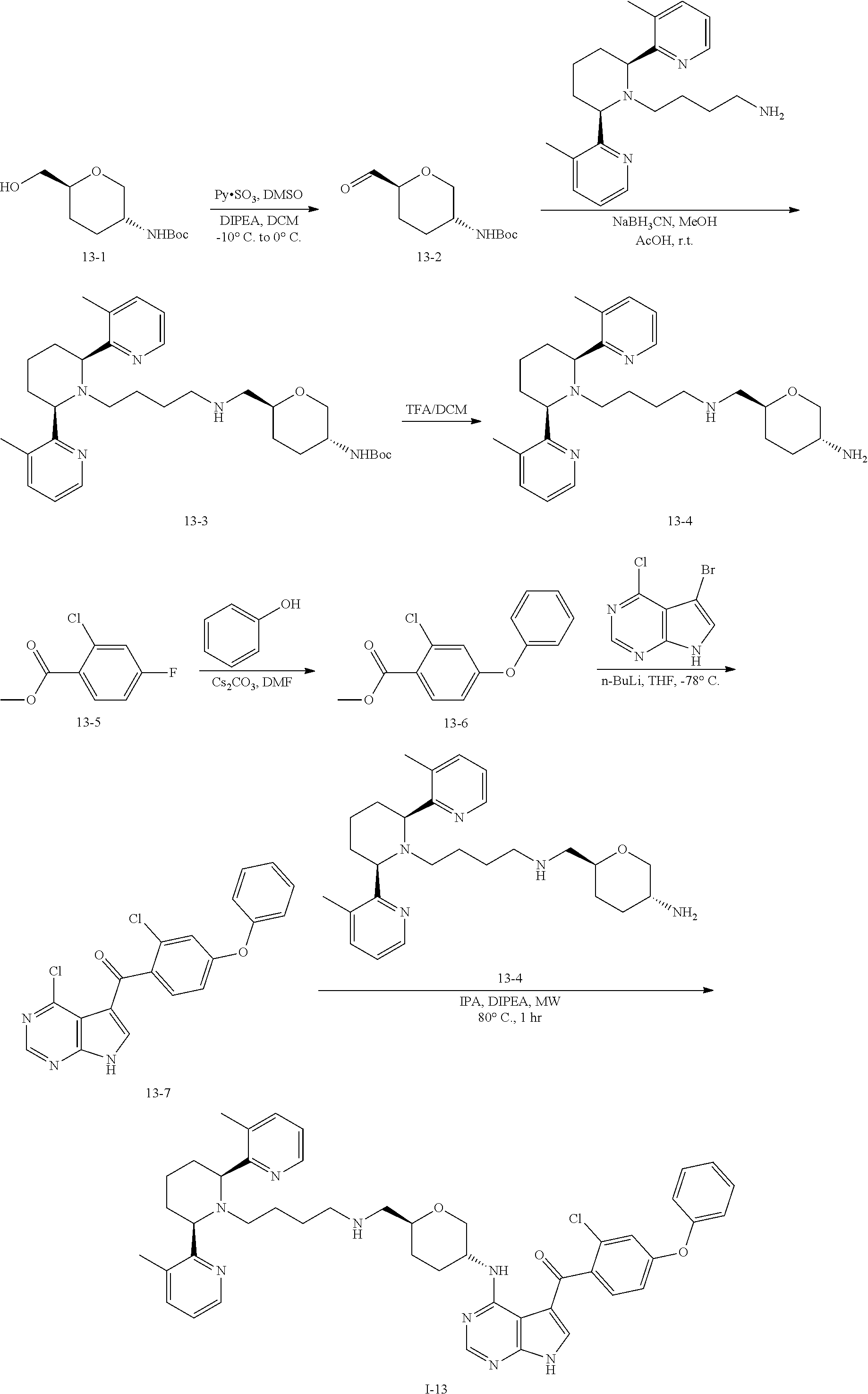

### 13.1 the Synthesis of 13-2

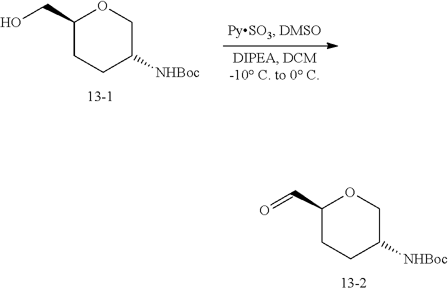

To a solution of tert-butyl (3R, 6S)-6-(hydroxymethyl)tetrahydro-2H-pyran-3-ylcarbamate (13-1, 1480 mg, 6.406 mmol) was slowly added a solution of DIPEA (3.4 mL) in DCM (5 mL) at −10° C. The mixture was stirred for 0.5 h. Then a solution of Py·SO₃ (2780 mg, 8.968 mmol) was added and the resulting mixture was stirred for 2 h at 0° C. After completion of the reaction indicated by TLC, the mixture was diluted with water, extracted with DCM. The organic layer was washed with brine, dried over Na₂SO₄, filtered and concentrated in vacuum to give the residue, 13-2, which is used directly in the next reaction without purification. 13.2 The synthesis of 13-3

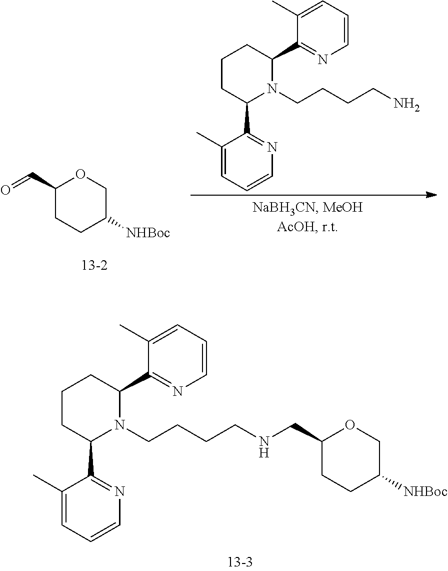

To a solution of tert-butyl (3R,6S)-6-formyltetrahydro-2H-pyran-3-ylcarbamate (13-2, 500 mg, 2.183 mmol), 4-((2R,6S)-2,6-bis(3-methylpyridin-2-yl)piperidin-1-yl) butan-1-amine (875 mg, 2.620 mmol) and AcOH (1 drops) in CH₃OH (10 mL) was added NaBH₃CN (140 mg, 4.366 mmol), then the mixture was stirred at room temperature overnight. After completion of the reaction indicated by LCMS, the reaction mixture was alkalized to pH=8 with NaHCO₃ aqueous solution and concentrated to give the residue, which was purified by column chromatography and prep-HPLC to give tert-butyl (3R,6S)-6-((4-((2R,6S)-2,6-bis(3-methylpyridin-2-yl)piperidin-1-yl)butylamino) methyl)tetrahydro-2H-pyran-3-ylcarbamate (13-3, 355 mg, 30%) as yellow brown syrup. LCMS (Agilent LCMS 1200-6120, Column: Waters X-Bridge C18 (50 mm*4.6 mm*3.5 μm); Column Temperature: 40° C.; Flow Rate: 2.0 mL/min; Mobile Phase: from 95% [water+10 mM NH₄HCO₃] and 5% [CH₃CN] to 0% [water+10 mM NH₄HCO₃] and 100% [CH₃CN] in 1.6 min, then under this condition for 1.4 min, finally changed to 95% [water+10 mM NH₄HCO₃] and 5% [CH₃CN] in 0.1 min and under this condition for 0.7 min.) Purity: 73%. Rt=1.75 min; MS Calcd.: 551.4; MS Found: 552.1 [M+H]⁺.

### 13-3 the Synthesis of 13-4

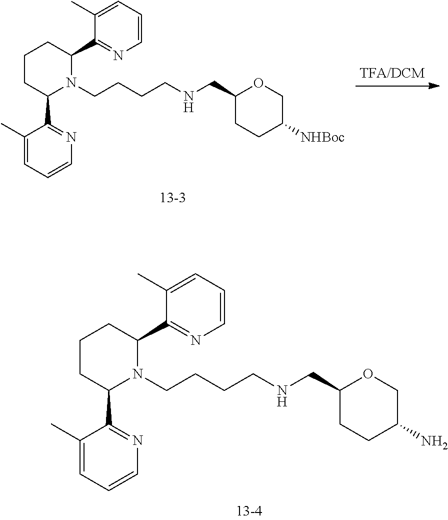 [SMILES: Cc1cccnc1[C@H]1CCC[C@@H](c2ncccc2C)N1CCCCNC[C@@H]1CC[C@@H](NC(=O)OC(C)(C)C)CO1 ; Cc1cccnc1[C@H]1CCC[C@@H](c2ncccc2C)N1CCCCNC[C@@H]1CC[C@@H](N)CO1]

To a solution of tert-butyl (3R,6S)-6-((4-((2R,6S)-2,6-bis(3-methylpyridin-2-yl)piperidin-1-yl)butylamino) methyl)tetrahydro-2H-pyran-3-ylcarbamate (13-3, 100 mg, 0.1841 mmol) in dry DCM (8 mL) was added TFA (2 mL), then the mixture was stirred at room temperature overnight. After completion of the reaction indicated by LCMS, the mixture was concentrated in vacuum, diluted with water, adjusted to pH=9 by NaOH solution and extracted with DCM/MeOH=30:1 (15 mL×3). The organic layer was washed with brine, dried over Na₂SO₄, filtered and concentrated in vacuum to give the residue, which was purified by prep-HPLC to give (3R,6S)-6-((4-((2R,6S)-2,6-bis(3-methylpyridin-2-yl)piperidin-1-yl)butylamino) methyl)tetrahydro-2H-pyran-3-amine (13-4, 61 mg, 75%) as colorless solid. LCMS (Agilent LCMS 1200-6120, Column: Waters X-Bridge C18 (50 mm*4.6 mm*3.5 μm); Column Temperature: 40° C.; Flow Rate: 2.0 mL/min; Mobile Phase: from 95% [water+10 mM NH₄HCO₃] and 5% [CH₃CN] to 0% [water+10 mM NH₄HCO₃] and 100% [CH₃CN] in 1.6 min, then under this condition for 1.4 min, finally changed to 95% [water+10 mM NH₄HCO₃] and 5% [CH₃CN] in 0.1 min and under this condition for 0.7 min.) Purity: 100.00%. Rt=2.015 min; MS Calcd.: 451.3; MS Found: 451.2 [M+H]⁺. HPLC (Agilent HPLC 1200, Column: L-column2 ODS (150 mm*4.6 mm*5.0 μm); Column Temperature: 40° C.; Flow Rate: 1.0 mL/min; Mobile Phase: from 90% [(total 10 mM AcONH₄) H₂O/MeCN=900/100 (v/v)] and 10% [total 10 mM AcONH₄) H₂O/MeCN=100/900 (v/v)] to 15% [total 10 mM AcONH₄) H₂O/MeCN=900/100 (v/v)] and 85% [total 10 mM AcONH₄) H₂O/MeCN=100/900 (v/v)] in 5 min, then under this condition for 10 min, finally changed to 90% [(total 10 mM AcONH₄) H₂O/MeCN=900/100 (v/v)] and 10% [total 10 mM AcONH₄) H₂O/MeCN=100/900 (v/v)] in 0.1 min and under this condition for 5 min. Purity: 100.00%, Rt=10.347 min; MS Calcd.: 451.3; MS Found: 451.2 [M+H]⁺. ¹H NMR (400 MHZ, CDCl₃)δ: 8.56 (s, 2H), 7.43-7.41 (m, 2H), 7.07-7.05 (m, 2H), 4.01-4.00 (d, J=2.4 Hz, 2H), 3.99-3.87 (m, 1H), 3.31-3.27 (m, 1H), 2.98-2.92 (t, J=10.8 Hz, 1H), 2.80-2.72 (m, 1H), 2.62 (s, 2H), 2.46-2.42 (m, 6H), 2.21-2.12 (m, 4H), 2.05-1.95 (m, 4H), 1.68-1.65 (d, J=1.2 Hz, 2H), 1.60-1.45 (m, 2H), 1.38-1.15 (m, 2H), 0.81 (s, 4H).

### 13-4 the Synthesis of 13-6

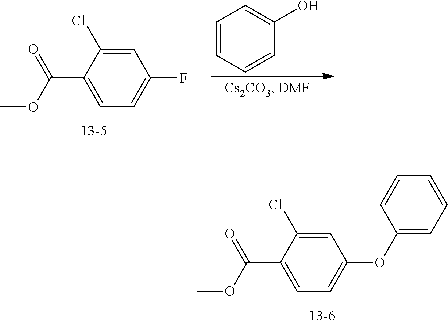

To a solution of methyl 2-chloro-4-fluorobenzoate (13-5, 33 g, 175.53 mmol) and phenol (20 g, 212.74 mmol) in DMF (200 mL) was added Cs₂CO₃ (85 g, 260.90 mmol), then the mixture was stirred at 90° C. for 3 h. After completion of the reaction indicated by LCMS, the reaction mixture was concentrated to give the residue, which was purified by column chromatography to give methyl 2-chloro-4-phenoxybenzoate (13-6, 39 g, 85%) as colorless liquid.

LCMS (Agilent LCMS 1200-6120, Column: Waters X-Bridge C18 (50 mm*4.6 mm*3.5 μm); Column Temperature: 40° C.; Flow Rate: 2.0 mL/min; Mobile Phase: from 95% [water+10 mM NH₄HCO₃] and 5% [CH₃CN] to 0% [water+10 mM NH₄HCO₃] and 100% [CH₃CN] in 1.6 min, then under this condition for 1.4 min, finally changed to 95% [water+10 mM NH₄HCO₃] and 5% [CH₃CN] in 0.1 min and under this condition for 0.7 min.) Purity: 80.92%. Rt=1.909 min; MS Calcd.: 262.0; MS Found: 263.0 [M+H]⁺.

### 13.5 the Synthesis of 13-7

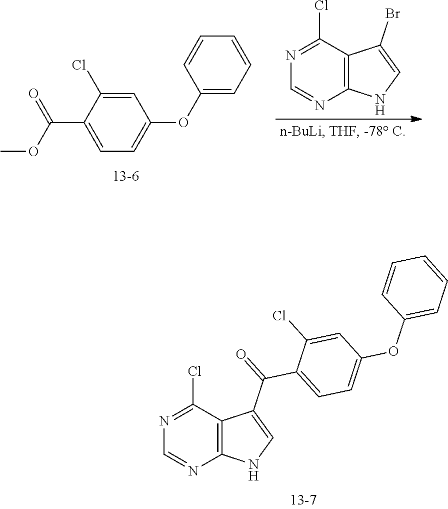

To a stirred solution of 5-bromo-4-chloro-7H-pyrrolo[2,3-d]pyrimidine (16.7 g, 72.32 mmol) in THF (400 mL) was added nBuLi (2.5M in THF, 60 mL, 151.87 mmol) at −78° C. under an atmosphere of nitrogen. The mixture was stirred at −78° C. for 50 min and methyl 2-chloro-4-phenoxybenzoate (13-6, 20 g, 75.94 mmol) was then added at the same temperature. After stirring at −78° C. for 50 min, the mixture was quenched with 1N HCl (150 mL), warmed to room temperature. After completion of the reaction indicated by LCMS, the reaction mixture was concentrated to give the residue, which was purified by column chromatography to give (2-chloro-4-phenoxyphenyl) (4-chloro-7H-pyrrolo[2,3-d]pyrimidin-5-yl) methanone (13-7, 11.6 g, 42%) as colorless solid. LCMS (Agilent LCMS 1200-6120, Column: Waters X-Bridge C18 (50 mm*4.6 mm*3.5 μm); Column Temperature: 40° C.; Flow Rate: 2.0 mL/min; Mobile Phase: from 95% [water+10 mM NH₄HCO₃] and 5% [CH₃CN] to 0% [water+10 mM NH₄HCO₃] and 100% [CH₃CN] in 1.6 min, then under this condition for 1.4 min, finally changed to 95% [water+10 mM NH₄HCO₃] and 5% [CH₃CN] in 0.1 min and under this condition for 0.7 min.) Purity: 100%. Rt=1.856 min; MS Calcd.: 383.0; MS Found: 383.9 [M+H]⁺.

### 13.6 the Synthesis of Compound I-13

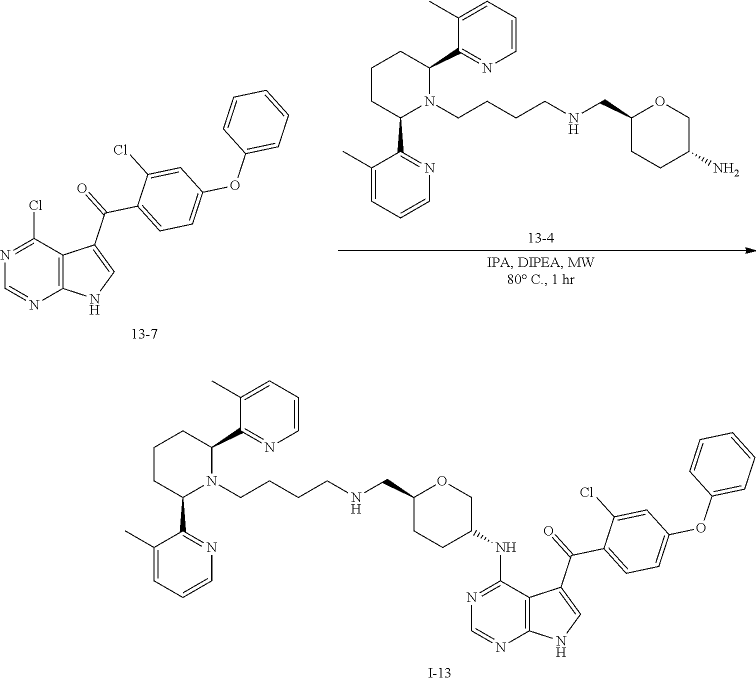

To a solution of (3R,6S)-6-((4-((2R,6S)-2,6-bis(3-methylpyridin-2-yl)piperidin-1-yl)butylamino) methyl)tetrahydro-2H-pyran-3-amine (13-4, 40 mg, 0.0888 mmol) and (2-chloro-4-phenoxyphenyl) (4-chloro-7H-pyrrolo[2,3-d]pyrimidin-5-yl) methanone (13-7, 34 mg, 0.0888 mmol) in IPA (4 mL) was added DIPEA (35 mg, 0.266 mmol) and stirred at 80° C. for 1 h under microwave irradiation. After completion of the reaction indicated by LCMS, the reaction mixture was concentrated to give the residue, which was purified by column chromatography and prep-HPLC to give (4-((3R,6S)-6-((4-((2R,6S)-2,6-bis(3-methylpyridin-2-yl)piperidin-1-yl)butylamino) methyl)tetrahydro-2H-pyran-3-ylamino)-7H-pyrrolo[2,3-d]pyrimidin-5-yl) (2-chloro-4-phenoxyphenyl) methanone (I-13, 25 mg, 36%) as colorless solid. ¹H NMR (400 MHZ, DMSO-d₆)δ: 8.26 (s, 2H), 8.20 (s, 1H), 7.58-7.47 (m, 4H), 7.40-7.37 (m, 3H), 7.29-7.25 (m, 1H), 7.20-7.18 (m, 3H), 7.18-7.14 (m, 1H), 7.13-7.00 (m, 3H), 4.14 (s, 2H), 3.66-3.62 (d, J=16 HZ, 1H), 3.18 (s, 2H), 3.07-3.05 (m, 2H), 2.86 (s, 1H), 2.74-2.69 (m, 2H), 2.37 (s, 6H), 2.14-2.09 (m, 4H), 1.96-1.92 (m, 1H), 1.79 (s, 1H), 1.58-1.56 (m, 2H), 1.41-1.39 (m, 2H), 1.04-1.00 (m, 2H), 0.40 (s, 2H), −0.14 (s, 2H).

### Example 14: Synthesis of Compound I-14

### Synthetic Scheme of Compound I-14

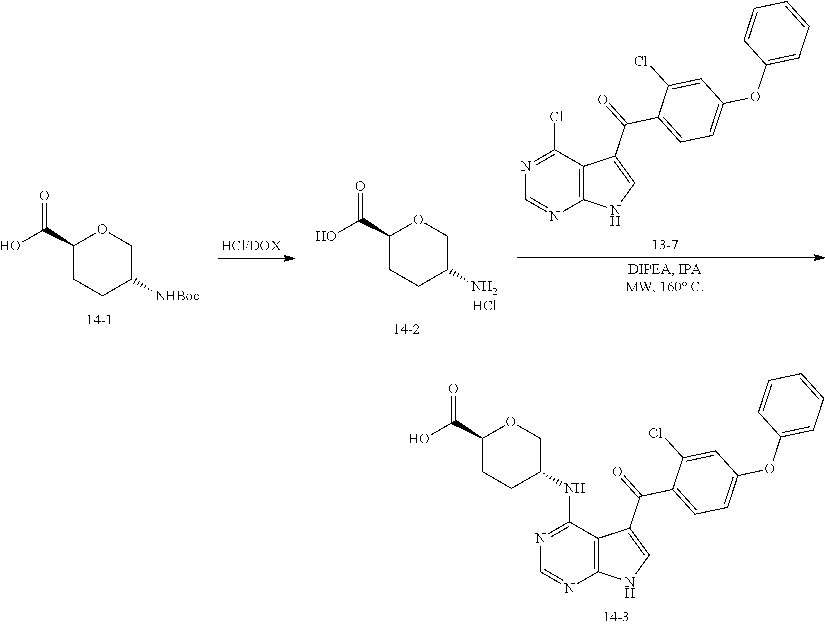

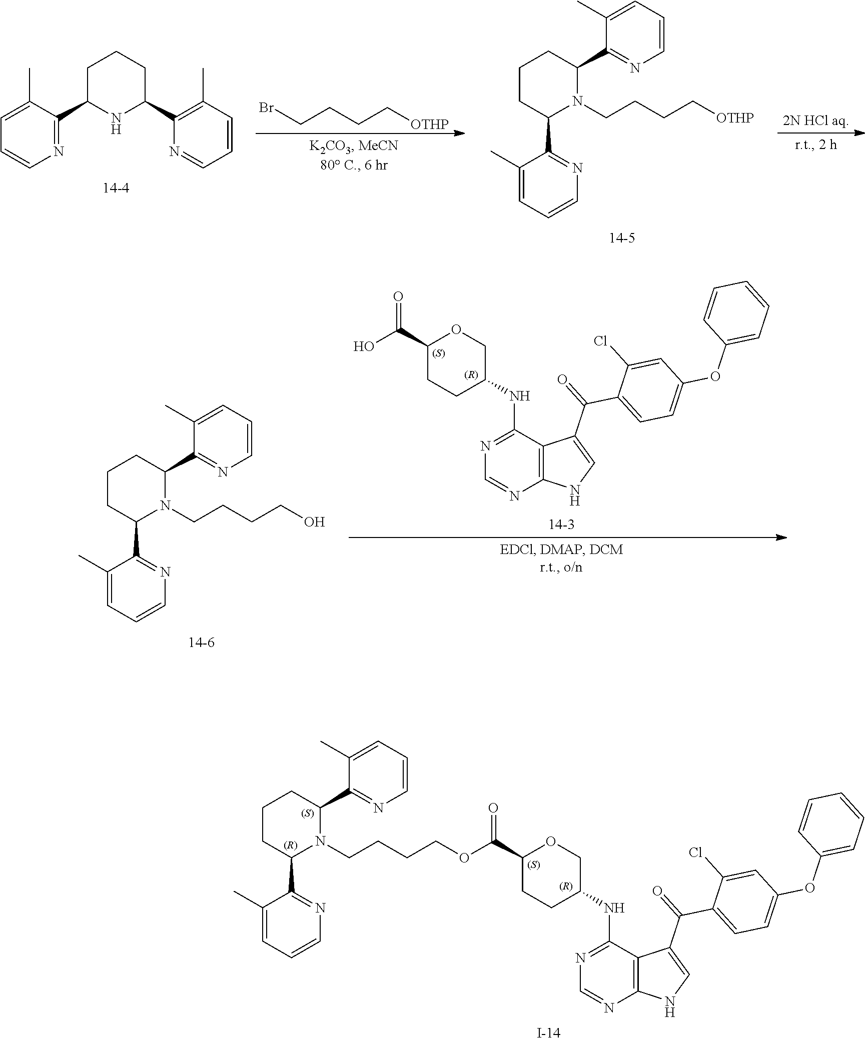

### 14.1 the Synthesis of 14-2

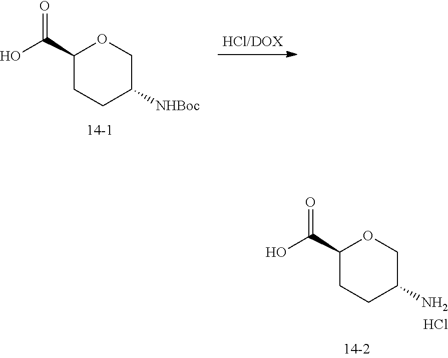

A solution of (2S, 5R)-5-(tert-butoxycarbonylamino)tetrahydro-2H-pyran-2-carboxylic acid (14-1, 2.45 g, 10.0 mmol) in HCl/DOX (16 mL) was stirred at room temperature overnight. The mixture was concentrated under reduced pressure to give 14-2 (2.0 g, >100.00%), Which was used in the next step without further purification.

### 14.2 the Synthesis of 14-3

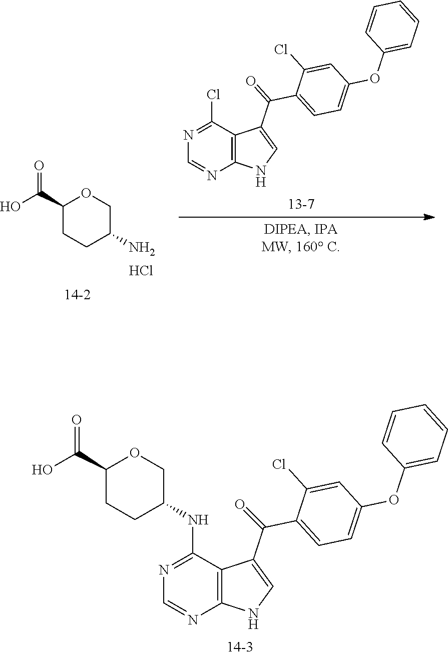 [SMILES: N[C@@H]1CC[C@@H](C(=O)O)OC1 ; O=C(c1ccc(Oc2ccccc2)cc1Cl)c1c[nH]c2ncnc(Cl)c12 ; Cl ; O=C(c1ccc(Oc2ccccc2)cc1Cl)c1c[nH]c2ncnc(N[C@@H]3CC[C@@H](C(=O)O)OC3)c12]

To a solution of (2S,5R)-5-aminotetrahydro-2H-pyran-2-carboxylic acid hydrochloride (14-2, 471 mg, 2.60 mmol) and (2-chloro-4-phenoxyphenyl) (4-chloro-7H-pyrrolo[2,3-d]pyrimidin-5-yl) methanone (13-7, 1.0 g, 2.60 mmol) in IPA (15 mL) was added DIPEA (1.0 g, 7.80 mmol) and stirred at 80° C. for 1 h under microwave irradiation. After completion of the reaction indicated by LCMS, the reaction mixture was concentrated to give the residue, which was purified by column chromatography and prep-HPLC to give (2S,5R)-5-(5-(2-chloro-4-phenoxybenzoyl)-7H-pyrrolo[2,3-d]pyrimidin-4-ylamino)tetrahydro-2H-pyran-2-carboxylic acid (14-3, 650 mg, 51%) as colorless solid. LCMS (Agilent LCMS 1200-6120, Column: Waters X-Bridge C18 (50 mm*4.6 mm*3.5 μm); Column Temperature: 40° C.; Flow Rate: 2.0 mL/min; Mobile Phase: from 95% [water+10 mM NH₄HCO₃] and 5% [CH₃CN] to 0% [water+10 mM NH₄HCO₃] and 100% [CH₃CN] in 1.6 min, then under this condition for 1.4 min, finally changed to 95% [water+10 mM NH₄HCO₃] and 5% [CH₃CN] in 0.1 min and under this condition for 0.7 min.) Purity: 100.00%. Rt=1.495 min; MS Calcd.: 492.1; MS Found: 493.0 [M+H]⁺. HPLC (Agilent HPLC 1200, Column: L-column2 ODS (150 mm*4.6 mm*5.0 μm); Column Temperature: 40° C.; Flow Rate: 1.0 mL/min; Mobile Phase: from 90% [(total 10 mM AcONH₄) H₂O/MeCN=900/100 (v/v)] and 10% [total 10 mM AcONH₄) H₂O/MeCN=100/900 (v/v)] to 15% [total 10 mM AcONH₄) H₂O/MeCN=900/100 (v/v)] and 85% [total 10 mM AcONH₄) H₂O/MeCN=100/900 (v/v)] in 5 min, then under this condition for 10 min, finally changed to 90% [(total 10 mM AcONH₄) H₂O/MeCN=900/100 (v/v)] and 10% [total 10 mM AcONH₄) H₂O/MeCN=100/900 (v/v)] in 0.1 min and under this condition for 5 min. Purity: 100.00%, Rt=6.888 min; MS Calcd.: 492.1; MS Found: 493.0 [M+H]⁺. ¹H NMR (400 MHZ, DMSO-d₆)δ: 8.68 (d, J=8.4 Hz, 1H), 8.26 (s, 1H), 7.63 (s, 1H), 7.58 (d, J=8.4 Hz, 2H), 7.57-7.46 (m, 2H), 7.26 (t, J=8.4 Hz, 1H), 7.19 (dd, J=2.8 Hz, 4.4 Hz, 3H), 7.02 (dd, J=2.0 Hz, 8.4 Hz, 1H), 4.18 (d, J=8.4 Hz, 2H), 2.15 (d, J=1.6 Hz, 1H), 2.05 (s, 1H), 1.68 (t, J=8.8 Hz, 2H).

### 14.3 the Synthesis of 14-5

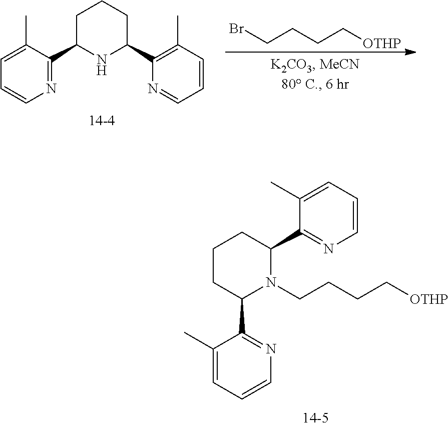

To a solution of (2R,6S)-2,6-bis(3-methylpyridin-2-yl)piperidine (14-4, 500 mg, 1.871 mmol) in MeCN (20 mL) was added 2-(4-bromobutoxy)tetrahydro-2H-pyran (666 mg, 2.807 mmol) and K₂CO₃ (259 mg, 1.871 mmol), then the mixture was stirred at 80° C. for 6 h. After completion of the reaction indicated by LCMS, the mixture was filtered and poured into water (15 mL) and extracted with DCM (50 mL×3). The organic layer was dried over Na₂SO₄, filtered and concentrated in vacuum to give a residue, which was purified by column chromatography (DCM:MeOH=30:1) to give the desired product (14-5, 650 mg, 82%) as yellow oil.

### 14.4 the Synthesis of 14-6

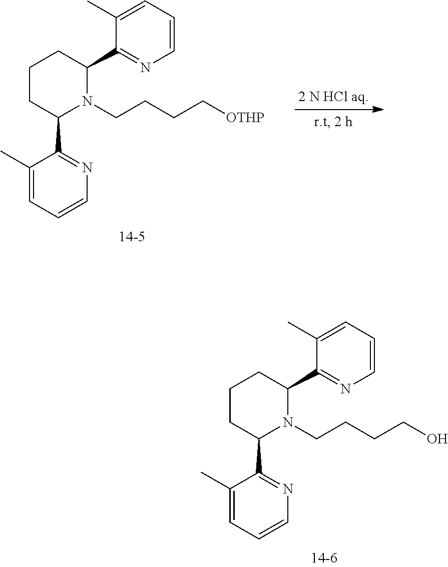

To a solution of 2,2′-((2R,6S)-1-(4-(tetrahydro-2H-pyran-2-yloxy)butyl)piperidine-2,6-diyl)bis(3-methylpyridine) (14-5, 650 mg, 1.536 mmol) in EtOH (15 mL) was added 2N HCl (6 mL), then the mixture was stirred at room temperature for 2 h. After completion of the reaction indicated by LCMS, the mixture was adjusted pH=8 by NaHCO₃ solution and extracted with DCM (50 mL×3). The organic layer was dried over Na₂SO₄, filtered and concentrated in vacuum to give a residue, which was purified by prep-HPLC to give the desired product (14-6, 466 mg, 89%) as yellow solid.

### 14.5 the Synthesis of Compound I-14

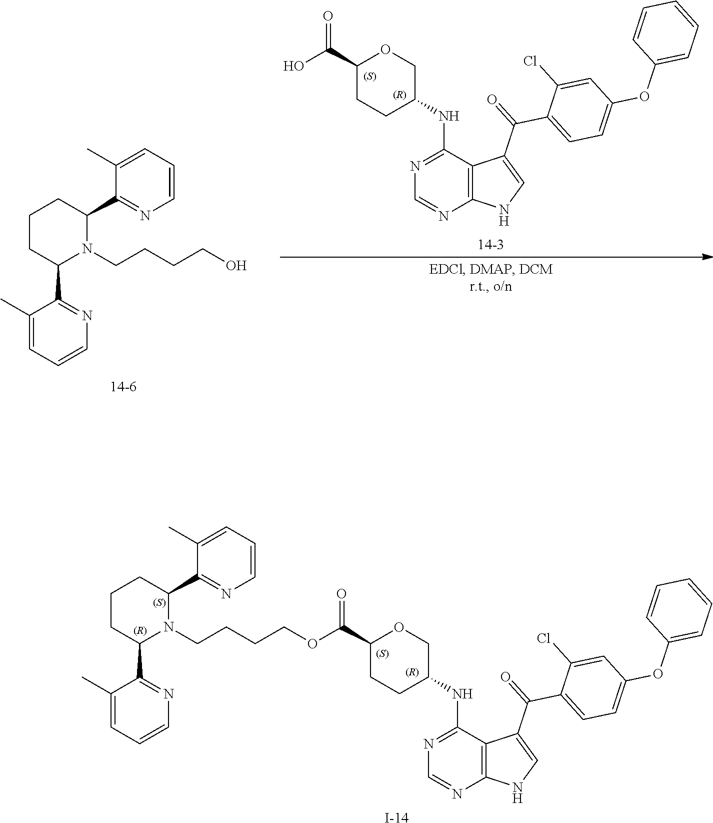

To a solution of (2S,5R)-5-(5-(2-chloro-4-phenoxybenzoyl)-7H-pyrrolo[2,3-d]pyrimidin-4-ylamino)tetrahydro-2H-pyran-2-carboxylic acid (14-3, 400 mg, 0.813 mmol) in DCM (15 mL) was added 4-((2R,6S)-2,6-bis(3-methylpyridin-2-yl)piperidin-1-yl) butan-1-ol (14-6, 276 mg, 0.813 mmol) and DMAP (198 mg, 1.626 mmoL) and EDCI (312 mg, 1.626 mmol), then the reaction mixture was stirred at room temperature for overnight. After completion of the reaction indicated by LCMS, the mixture was poured into water (15 mL) and extracted with DCM/MeOH-30:1 (40 mL×4). The organic layer was dried over Na₂SO₄, filtered and concentrated in vacuum to give a residue, which was purified by prep-HPLC to give the desired product (I-14, 50.06 mg, 7.6%) as white solid. LCMS (Agilent LCMS 1200-6120, Column: Waters X-Bridge C18 (50 mm*4.6 mm*3.5 μm); Column Temperature: 40° C.; Flow Rate: 2.0 mL/min; Mobile Phase: from 95% [water+10 mM NH₄HCO₃] and 5% [CH₃CN] to 0% [water+10 mM NH₄HCO₃] and 100% [CH₃CN] in 1.6 min, then under this condition for 1.4 min, finally changed to 95% [water+10 mM NH₄HCO₃] and 5% [CH₃CN] in 0.1 min and under this condition for 0.7 min.) Purity: 100.00%. Rt=2.244 min; MS Calcd.: 813.3; MS Found: 813.8 [M+H]⁺. HPLC (Agilent HPLC 1200, Column: L-column2 ODS (150 mm*4.6 mm*5.0 μm); Column Temperature: 40° C.; Flow Rate: 1.0 mL/min; Mobile Phase: from 90% [(total 10 mM AcONH₄) H₂O/MeCN=900/100 (v/v)] and 10% [total 10 mM AcONH₄) H₂O/MeCN=100/900 (v/v)] to 15% [total 10 mM AcONH₄) H₂O/MeCN=900/100 (v/v)] and 85% [total 10 mM AcONH₄) H₂O/MeCN=100/900 (v/v)] in 5 min, then under this condition for 10 min, finally changed to 90% [(total 10 mM AcONH₄) H₂O/MeCN=900/100 (v/v)] and 10% [total 10 mM AcONH₄) H₂O/MeCN=100/900 (v/v)] in 0.1 min and under this condition for 5 min. Purity: 100.00%, Rt=11.469 min; MS Calcd.: 813.3; MS Found: 813.8 [M+H]. ¹H NMR (400 MHZ, DMSO-d₆) δ: 12.78 (s, 1H), 8.69 (d, J=7.2 Hz, 1H), 8.35 (s, 2H), 8.27 (s, 1H), 7.65 (s, 1H), 7.59-7.54 (m, 3H), 7.51-7.46 (m, 2H), 7.28-7.24 (m, 1H), 7.20-7.16 (m, 5H), 7.02 (dd, J=2.4, 8.4 Hz, 1H), 4.22-4.13 (m, 4H), 3.98 (d, J=9.6 Hz, 1H), 3.42 (d, J=3.2 Hz, 1H), 3.27 (d, J=9.6 Hz, 2H), 2.54 (s, 6H), 2.33-2.24 (m, 2H), 2.16-2.01 (m, 3H), 1.97-1.90 (m, 2H), 1.75-1.67 (m, 2H), 1.61-1.50 (m, 3H), 0.69 (d, J=4.8 Hz, 2H), 0.22-0.18 (m, 2H).

### Example 15: Synthesis of Compound I-15

### Synthetic Scheme of Compound I-15

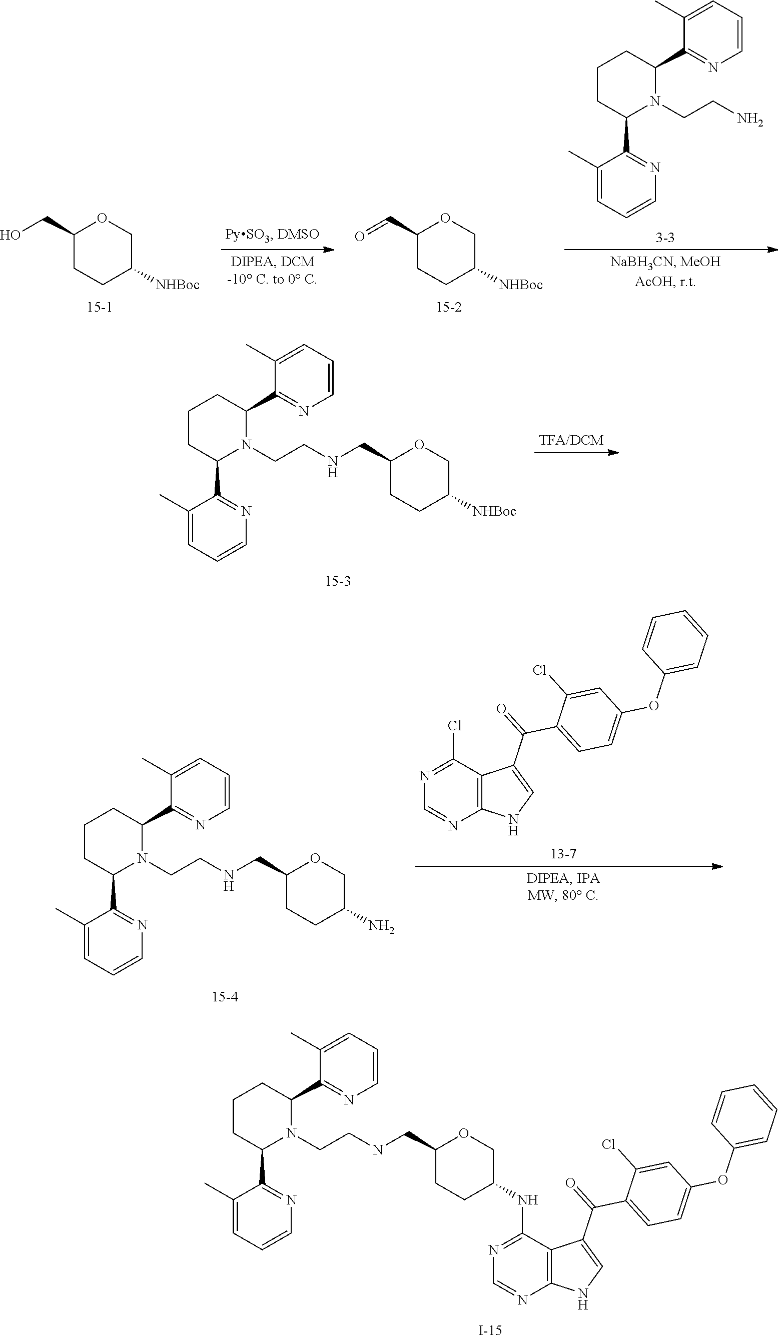

### 15.1 the Synthesis of 15-2

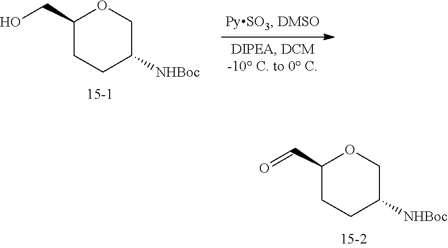

To a solution of tert-butyl (3R, 6S)-6-(hydroxymethyl)tetrahydro-2H-pyran-3-ylcarbamate (15-1, 1480 mg, 6.406 mmol) was slowly added a solution of DIPEA (3.4 mL) in DCM (5 mL) at −10° C. The mixture was stirred for 0.5 h. Then a solution of Py·SO₃ (2780 mg, 8.968 mmol) was added and the resulting mixture was stirred for 2 h at 0° C. After completion of the reaction indicated by TLC, the mixture was diluted with water, extracted with DCM. The organic layer was washed with brine, dried over Na₂SO₄, filtered and concentrated in vacuum to give the residue, 15-2, which is used directly in the next reaction without purification.

### 15.2 the Synthesis of 15-3

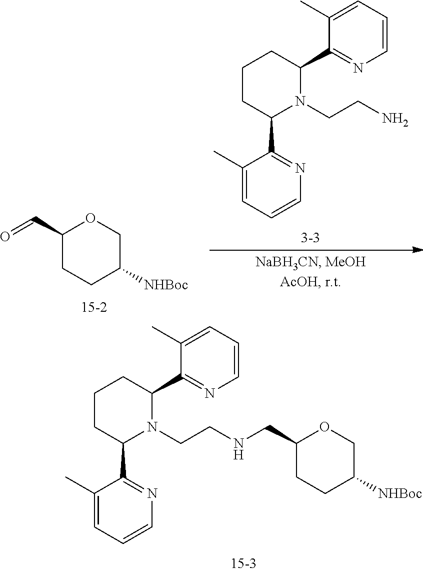

To a solution of tert-butyl (3R,6S)-6-formyltetrahydro-2H-pyran-3-ylcarbamate (15-2, 400 mg, 1.747 mmol), 2-((2R,6S)-2,6-bis(3-methylpyridin-2-yl)piperidin-1-yl) ethanamine (3-3, 271 mg, 0.874 mmol) and AcOH (1 drops) in CH₃OH (10 mL) was added NaBH₃CN (165 mg, 2.622 mmol), then the mixture was stirred at room temperature overnight. After completion of the reaction indicated by LCMS, the reaction mixture was alkalixed to pH=8 with NaHCO₃ aqueous solution and concentrated to give the residue, which was purified by column chromatography to give tert-butyl (3R,6S)-6-((2-((2R,6S)-2,6-bis(3-methylpyridin-2-yl)piperidin-1-yl)ethylamino) methyl)tetrahydro-2H-pyran-3-ylcarbamate (15-3, 350 mg, 76%) as yellow brown syrup. LCMS (Agilent LCMS 1200-6120, Column: Waters X-Bridge C18 (50 mm*4.6 mm*3.5 μm); Column Temperature: 40° C.; Flow Rate: 2.0 mL/min; Mobile Phase: from 95% [water+10 mM NH₄HCO₃] and 5% [CH₃CN] to 0% [water+10 mM NH₄HCO₃] and 100% [CH₃CN] in 1.6 min, then under this condition for 1.4 min, finally changed to 95% [water+10 mM NH₄HCO₃] and 5% [CH₃CN] in 0.1 min and under this condition for 0.7 min.) Purity: 100%. Rt=1.985 min; MS Calcd.: 523.4; MS Found: 524.4 [M+H]⁺.

### 15.3 the Synthesis of 15-4

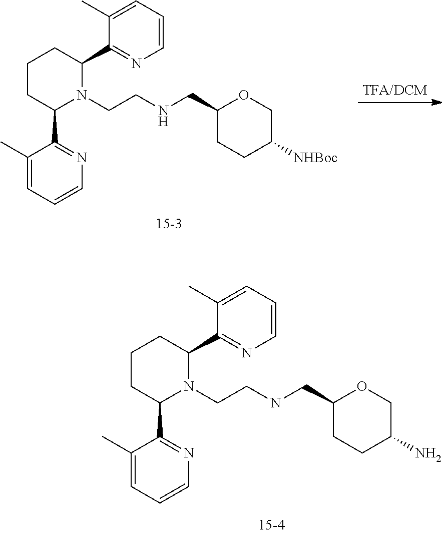

To a solution of tert-butyl (3R,6S)-6-((2-((2R,6S)-2,6-bis(3-methylpyridin-2-yl)piperidin-1-yl)ethylamino)methyl)tetrahydro-2H-pyran-3-ylcarbamate (15-3, 350 mg, 0.669 mmol) in dry DCM (8 mL) was added TFA (2 mL), then the mixture was stirred at room temperature overnight. After completion of the reaction indicated by LCMS, the mixture was concentrated in vacuum, diluted with water, adjusted to pH=9 by NaOH solution and extracted with DCM/MeOH=30:1 (15 mL×3). The organic layer was washed with brine, dried over Na₂SO₄, filtered and concentrated in vacuum to give the residue, which was purified by prep-HPLC to give (3R,6S)-6-((2-((2R,6S)-2,6-bis(3-methylpyridin-2-yl)piperidin-1-yl)ethylamino)methyl)tetrahydro-2H-pyran-3-amine (15-4, 200 mg, 71%) as colorless solid. LCMS (Agilent LCMS 1200-6120, Column: Waters X-Bridge C18 (50 mm*4.6 mm*3.5 μm); Column Temperature: 40° C.; Flow Rate: 2.0 mL/min; Mobile Phase: from 95% [water+10 mM NH₄HCO₃] and 5% [CH₃CN] to 0% [water+10 mM NH₄HCO₃] and 100% [CH₃CN] in 1.6 min, then under this condition for 1.4 min, finally changed to 95% [water+10 mM NH₄HCO₃] and 5% [CH₃CN] in 0.1 min and under this condition for 0.7 min.) Purity: 87%. Rt=1.618 min; MS Calcd.: 423.3; MS Found: 424.1 [M+H]⁺.

### 15.4 the Synthesis of Compound I-15

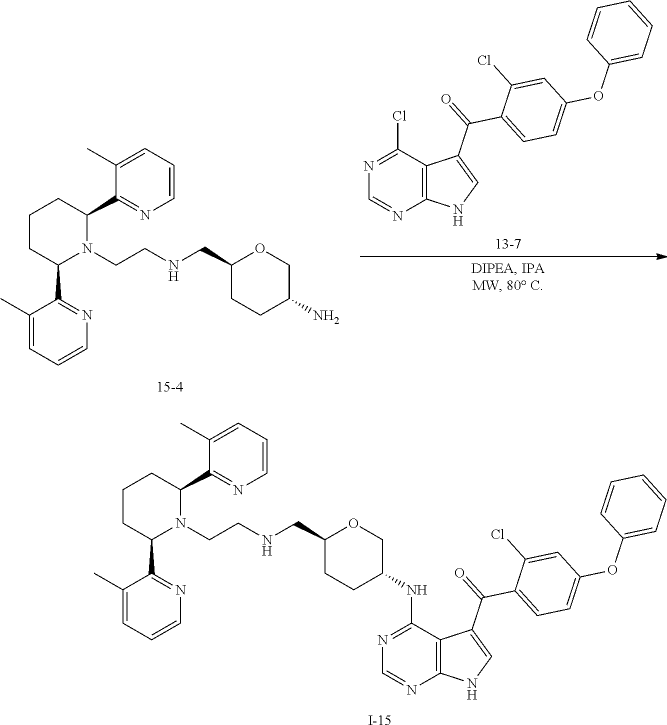

To a solution of (3R,6S)-6-((2-((2R,6S)-2,6-bis(3-methylpyridin-2-yl)piperidin-1-yl)ethylamino)methyl)tetrahydro-2H-pyran-3-amine (15-4, 100 mg, 0.236 mmol) and (2-chloro-4-phenoxyphenyl) (4-chloro-7H-pyrrolo[2,3-d]pyrimidin-5-yl) methanone (13-7, 95 mg, 0.248 mmol) in IPA (4 mL) was added DIPEA (91 mg, 0.708 mmol), then the mixture was stirred at 80° C. for 1 h under microwave irradiation. After completion of the reaction indicated by LCMS, the reaction mixture was concentrated to give the residue, which was purified by column chromatography and prep-HPLC to give (4-((3R,6S)-6-((2-((2R,6S)-2,6-bis(3-methylpyridin-2-yl)piperidin-1-yl)ethylamino)methyl)tetrahydro-2H-pyran-3-ylamino)-7H-pyrrolo[2,3-d]pyrimidin-5-yl) (2-chloro-4-phenoxyphenyl) methanone (I-15, 6 mg, 4%) as colorless solid. LCMS (Agilent LCMS 1200-6120, Column: Waters X-Bridge C18 (50 mm*4.6 mm*3.5 μm); Column Temperature: 40° C.; Flow Rate: 2.0 mL/min; Mobile Phase: from 95% [water+10 mM NH₄HCO₃] and 5% [CH₃CN] to 0% [water+10 mM NH₄HCO₃] and 100% [CH₃CN] in 1.6 min, then under this condition for 1.4 min, finally changed to 95% [water+10 mM NH₄HCO₃] and 5% [CH₃CN] in 0.1 min and under this condition for 0.7 min.) Purity: 100.00%. Rt=1.93 min; MS Calcd.: 771.4; MS Found: 771.0 [M+H]⁺. HPLC (Agilent HPLC 1200, Column: L-column2 ODS (150 mm*4.6 mm*5.0 μm); Column Temperature: 40° C.; Flow Rate: 1.0 mL/min; Mobile Phase: from 90% [(total 10 mM AcONH₄) H₂O/MeCN=900/100 (v/v)] and 10% [total 10 mM AcONH₄) H₂O/MeCN=100/900 (v/v)] to 15% [total 10 mM AcONH₄) H₂O/MeCN=900/100 (v/v)] and 85% [total 10 mM AcONH₄) H₂O/MeCN=100/900 (v/v)] in 5 min, then under this condition for 10 min, finally changed to 90% [(total 10 mM AcONH₄) H₂O/MeCN=900/100 (v/v)] and 10% [total 10 mM AcONH₄) H₂O/MeCN=100/900 (v/v)] in 0.1 min and under this condition for 5 min. Purity: 97.99%, Rt=9.82 min; MS Calcd.: 762.4; MS Found: 771.0 [M+H]⁺. ¹H NMR (400 MHZ, DMSO-d₆)δ: 8.21-8.16 (d, J=20 Hz, 2H), 7.81 (s, 1H), 7.53-7.47 (m, 5H), 7.38-7.36 (m, 2H), 7.28-7.24 (m, 1H), 7.19-7.17 (d, J=8 Hz, 3H), 7.09-7.07 (m, 3H), 7.03-7.01 (d, J=8 Hz, 1H), 4.24-4.22 (m, 2H), 3.92 (s, 1H), 3.18 (s, 2H), 3.17-3.11 (m, 3H), 2.89 (s, 6H), 2.74-2.69 (m, 2H), 2.69-2.63 (m, 3H), 2.23-2.19 (m, 4H), 1.98-1.96 (m, 1H), 1.63-1.60 (m, 2H), 1.49-1.46 (m, 2H), 1.25-1.21 (m, 1H), 1.19-1.15 (m, 1H), 0.85-0.83 (m, 1H), 0.66-0.63 (m, 1H).

### Example 16: Synthesis of Compound I-16

### 16.1 the Synthesis of Compound I-16

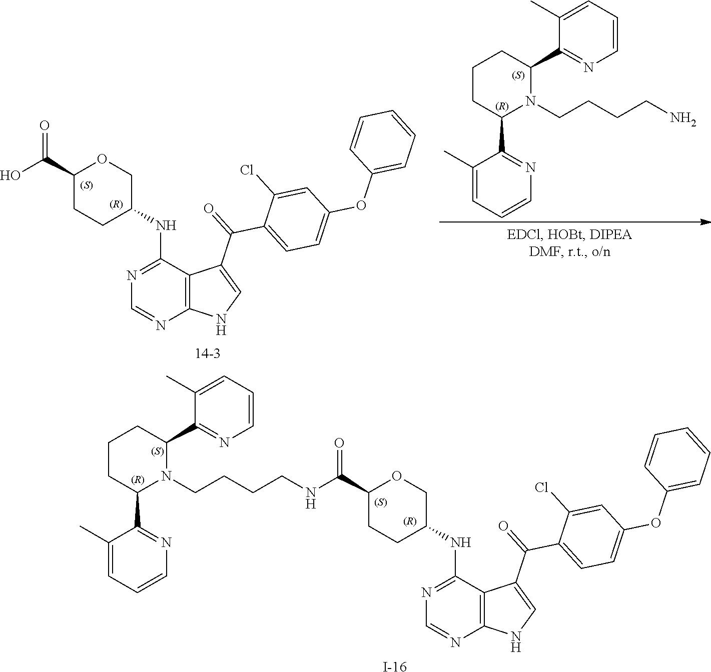

To a solution of (2S,5R)-5-(5-(2-chloro-4-phenoxybenzoyl)-7H-pyrrolo[2,3-d]pyrimidin-4-ylamino)tetrahydro-2H-pyran-2-carboxylic acid (14-3, 50 mg, 0.102 mmol) in DMF (2 mL) was added 4-((2R,6S)-2,6-bis(3-methylpyridin-2-yl)piperidin-1-yl) butan-1-amine (38 mg, 0.112 mmol) and DIPEA (39 mg, 0.305 mmoL), EDCI (29 mg, 0.152 mmol) and HOBT (21 mg, 0.152 mmol), then the reaction mixture was stirred at room temperature for overnight. After completion of the reaction indicated by LCMS, the reaction mixture was concentrated in vacuum to give a residue, which was purified by prep-HPLC to give the desired product (I-16, 19.54 mg, 24%) as yellow solid. LCMS (Agilent LCMS 1200-6120, Column: Waters X-Bridge C18 (50 mm*4.6 mm*3.5 μm); Column Temperature: 40° C.; Flow Rate: 2.0 mL/min; Mobile Phase: from 95% [water+10 mM NH₄HCO₃] and 5% [CH₃CN] to 0% [water+10 mM NH₄HCO₃] and 100% [CH₃CN] in 1.6 min, then under this condition for 1.4 min, finally changed to 95% [water+10 mM NH₄HCO₃] and 5% [CH₃CN] in 0.1 min and under this condition for 0.7 min.) Purity: 100.00%. Rt=2.342 min; MS Calcd.: 812.4; MS Found: 813.3 [M+H]⁺. HPLC (Agilent HPLC 1200, Column: L-column2 ODS (150 mm*4.6 mm*5.0 μm); Column Temperature: 40° C.; Flow Rate: 1.0 mL/min; Mobile Phase: from 90% [(total 10 mM AcONH₄) H₂O/MeCN=900/100 (v/v)] and 10% [total 10 mM AcONH₄) H₂O/MeCN=100/900 (v/v)] to 15% [total 10 mM AcONH₄) H₂O/MeCN=900/100 (v/v)] and 85% [total 10 mM AcONH₄) H₂O/MeCN=100/900 (v/v)] in 5 min, then under this condition for 10 min, finally changed to 90% [(total 10 mM AcONH₄) H₂O/MeCN=900/100 (v/v)] and 10% [total 10 mM AcONH₄) H₂O/MeCN=100/900 (v/v)] in 0.1 min and under this condition for 5 min. Purity: 100.00%, Rt=11.193 min; MS Calcd.: 812.4; MS Found: 813.3 [M+H]⁺. ¹H NMR (400 MHZ, DMSO-d₆) δ: 8.62 (d, J=7.2 Hz, 1H), 8.35 (d, J=2.4 Hz, 2H), 8.26 (s, 1H), 7.63 (s, 1H), 7.55 (dd, J=8.8, 11.6 Hz, 3H), 7.48 (t, J=7.6 Hz, 2H), 7.26 (t, J=7.6 Hz, 1H), 7.19-7.16 (m, 6H), 7.02 (dd, J=2.4, 8.4 Hz, 1H), 4.20-4.16 (s, 4H), 3.72 (d, J=9.6 Hz, 1H), 3.22 (t, J=10.0 Hz, 1H), 2.55 (s, 6H), 2.25-2.12 (m, 4H), 2.10-2.04 (m, 2H), 2.01-1.92 (m, 3H), 1.68-1.56 (m, 3H), 1.51-1.48 (m, 2H), 1.45-1.38 (m, 1H), 0.55 (s, 2H), 0.25 (s, 1H).

### Example 17: Synthesis of Compound I-17

### 17.1 the Synthesis of Compound I-17

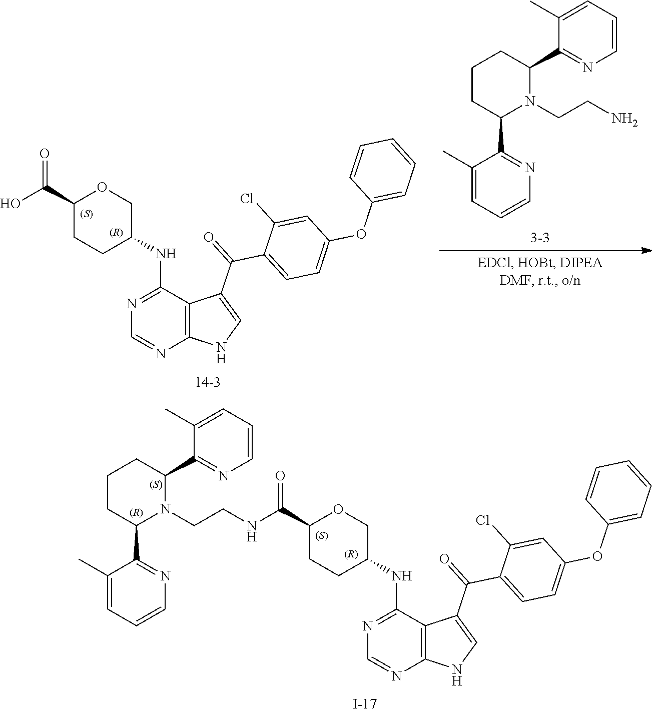

To a solution of (2S,5R)-5-(5-(2-chloro-4-phenoxybenzoyl)-7H-pyrrolo[2,3-d]pyrimidin-4-ylamino)tetrahydro-2H-pyran-2-carboxylic acid (14-3, 100 mg, 0.203 mmol) in DMF (4 mL) was added 2-((2R,6S)-2,6-bis(3-methylpyridin-2-yl)piperidin-1-yl) ethanamine (3-3, 95 mg, 0.305 mmol) and DIPEA (79 mg, 0.610 mmol), EDCI (59 mg, 0.305 mmol) and HOBT (41 mg, 0.305 mmol), then the reaction mixture was stirred at room temperature for overnight. After completion of the reaction indicated by LCMS, the reaction mixture was concentrated in vacuum to give a residue, which was purified by prep-HPLC to give the desired product (1-17, 44.10 mg, 28%) as off-white solid. LCMS (Agilent LCMS 1200-6120, Column: Waters X-Bridge C18 (50 mm*4.6 mm*3.5 μm); Column Temperature: 40° C.; Flow Rate: 2.0 mL/min; Mobile Phase: from 95% [water+10 mM NH₄HCO₃] and 5% [CH₃CN] to 0% [water+10 mM NH₄HCO₃] and 100% [CH₃CN] in 1.6 min, then under this condition for 1.4 min, finally changed to 95% [water+10 mM NH₄HCO₃] and 5% [CH₃CN] in 0.1 min and under this condition for 0.7 min.) Purity: 100.00%. Rt=1.990 min; MS Calcd.: 784.3; MS Found: 785.0 [M+H]⁺. HPLC (Agilent HPLC 1200, Column: L-column2 ODS (150 mm*4.6 mm*5.0 μm); Column Temperature: 40° C.; Flow Rate: 1.0 mL/min; Mobile Phase: from 90% [(total 10 mM AcONH₄) H₂O/MeCN=900/100 (v/v)] and 10% [total 10 mM AcONH₄) H₂O/MeCN=100/900 (v/v)] to 15% [total 10 mM AcONH₄) H₂O/MeCN=900/100 (v/v)] and 85% [total 10 mM AcONH₄) H₂O/MeCN=100/900 (v/v)] in 5 min, then under this condition for 10 min, finally changed to 90% [(total 10 mM AcONH₄) H₂O/MeCN=900/100 (v/v)] and 10% [total 10 mM AcONH₄) H₂O/MeCN=100/900 (v/v)] in 0.1 min and under this condition for 5 min. Purity: 98.56%, Rt=10.469 min; MS Calcd.: 784.3; MS Found: 784.8 [M+H]⁺. ¹H NMR (400 MHZ, DMSO-d₆) ô: 12.75 (s, 1H), 8.61 (d, J=7.2 Hz, 1H), 8.37 (s, 2H), 8.31 (s, 1H), 7.66 (s, 2H), 7.58 (d, J=8.4 Hz, 2H), 7.48 (t, J=8.4 Hz, 2H), 7.27 (d, J=7.6 Hz, 1H), 7.27-7.18 (m, 5H), 7.02 (dd, J=2.4, 8.4 Hz, 1H), 6.35 (s, 1H), 4.28 (dd, J=10.8, 32 Hz, 3H), 4.18-4.14 (m, 1H), 3.47 (d, J=10.8 Hz, 1H), 3.16 (t, J=10.0 Hz, 1H), 2.57 (t, J=10.4 Hz, 8H), 2.35-2.21 (m, 2H), 2.12-2.09 (m, 1H), 2.01-1.98 (m, 1H), 1.86-1.83 (m, 1H), 1.74-1.63 (m, 3H), 1.61-1.57 (m, 1H), 1.48 (d, J=12.8 Hz, 2H), 1.15-1.08 (m, 1H).

### Example 18: Synthesis of Compound I-18 and Additional Exemplary Compounds

### Synthetic Scheme of I-18

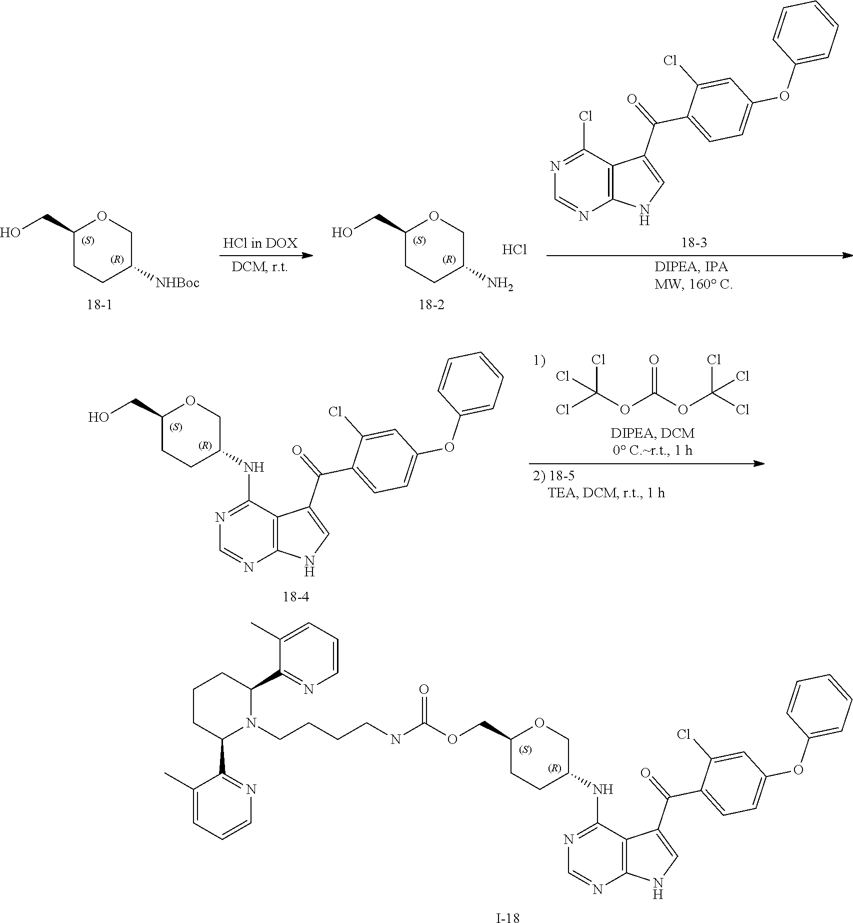

### The Synthesis of 18-2

To a solution of 18-1 (6 g, 25.97 mmol) in dry DCM (30 mL) was added 4N HCl in DOX (30 mL), then the mixture was stirred at room temperature overnight. After completion of the reaction indicated by TLC, the mixture was concentrated in vacuum to give 18-2 (4.2 g, 97%) as colorless solid.

### The Synthesis of 18-4

To a solution of 18-2 (1.00 g, 5.99 mmol) in IPA (8 mL) was added 18-3 (2.29 g, 5.99 mmol) and DIPEA (2.32 g, 17.97 mmol), then the mixture was stirred at 160° C. for 1 h under microwave irradiation. After completion of the reaction indicated by LCMS, the mixture was filtered and concentrated in vacuum to give a residue, which was purified by column chromatography (DCM/MeOH=10/1) to give the desired product (2.43 g, 85%) as white solid. LCMS (Agilent LCMS 1200-6120, Column: Waters X-Bridge C18 (50 mm*4.6 mm*3.5 μm); Column Temperature: 40° C.; Flow Rate: 2.0 mL/min; Mobile Phase: from 95% [water+10 mM NH₄HCO₃] and 5% [CH₃CN] to 0% [water+10 mM NH₄HCO₃] and 100% [CH₃CN] in 1.6 min, then under this condition for 1.4 min, finally changed to 95% [water+10 mM NH₄HCO₃] and 5% [CH₃CN] in 0.1 min and under this condition for 0.7 min.) Purity: 100%. Rt=1.771 min; MS Calcd.: 478.1; MS Found: 479.2 [M+H]⁺.

### The Synthesis of I-18

To a solution of triphosgene (176 mg, 0.593 mmol) in DCM (12 mL) was added a mixture of 18-4 (850 mg, 1.777 mmol) and DIPEA (230 mg, 1.777 mmoL) in DCM (10 mL) dropwise at 0° C. under N₂ atmosphere. Then the reaction mixture was stirred at room temperature for 2 h, followed by adding a solution of 18-5 (601 mg, 1.777 mmol) and TEA (180 mg, 1.777 mmol) in DCM (10 mL), then the mixture was stirred at room temperature for 1 h. After completion of the reaction indicated by LCMS, the reaction mixture was poured into sat. NaHCO₃ solution (30 mL) and extracted with DCM (30 mL×3), the organic layer was dried over Na₂SO₄, filtered and concentrated in vacuum to give a residue, which was purified by prep-HPLC to give the desired product (I-18, 637.30 mg, 42%) as off-white solid.

LCMS (Agilent LCMS 1200-6120, Column: Waters X-Bridge C18 (50 mm*4.6 mm*3.5 μm); Column Temperature: 40° C.; Flow Rate: 2.0 mL/min; Mobile Phase: from 95% [water+10 mM NH₄HCO₃] and 5% [CH₃CN] to 0% [water+10 mM NH₄HCO₃] and 100% [CH₃CN] in 1.6 min, then under this condition for 1.4 min, finally changed to 95% [water+10 mM NH₄HCO₃] and 5% [CH₃CN] in 0.1 min and under this condition for 0.7 min.) Purity: 99.07%. Rt=2.065 min; MS Calcd.: 842.4; MS Found: 843.4 [M+H]⁺. HPLC (Agilent HPLC 1200, Column: L-column2 ODS (150 mm*4.6 mm*5.0 μm); Column Temperature: 40° C.; Flow Rate: 1.0 mL/min; Mobile Phase: from 90% [(total 10 mM AcONH₄) H₂O/MeCN=900/100 (v/v)] and 10% [total 10 mM AcONH₄) H₂O/MeCN=100/900 (v/v)] to 15% [total 10 mM AcONH₄) H₂O/MeCN=900/100 (v/v)] and 85% [total 10 mM AcONH₄) H₂O/MeCN=100/900 (v/v)] in 5 min, then under this condition for 10 min, finally changed to 90% [(total 10 mM AcONH₄) H₂O/MeCN=900/100 (v/v)] and 10% [total 10 mM AcONH₄) H₂O/MeCN=100/900 (v/v)] in 0.1 min and under this condition for 5 min. Purity: 98.71%, Rt=11.094 min; MS Calcd.: 842.4; MS Found: 843.3 [M+H]⁺. ¹H NMR (400 MHZ, DMSO-d₆) δ: 12.7 (s, 1H), 8.59 (d, J=7.2 Hz, 1H), 8.34 (d, J=3.2 Hz, 2H), 8.25 (s, 1H), 7.64 (s, 1H), 7.57 (d, J=8.8 Hz, 1H), 7.53-7.45 (m, 4H), 7.27-7.24 (m, 1H), 7.19-7.14 (m, 5H), 7.02 (dd, J=2.4, 8.4 Hz, 1H), 6.82 (s, 1H), 4.15 (t, J=12.0 Hz, 4H), 3.92-3.82 (m, 2H), 3.51 (s, 1H), 3.11 (t, J=11.6 Hz, 1H), 2.54 (s, 6H), 2.33-2.20 (m, 2H), 2.20 (s, 3H), 2.13-2.11 (m, 2H), 1.94 (s, 1H), 1.72 (d, J=12.4 Hz, 1H), 1.65-1.56 (m, 2H), 1.50-1.35 (m, 3H), 0.53 (s, 2H), 0.24 (m, 2H).

### Synthetic Scheme for I-67

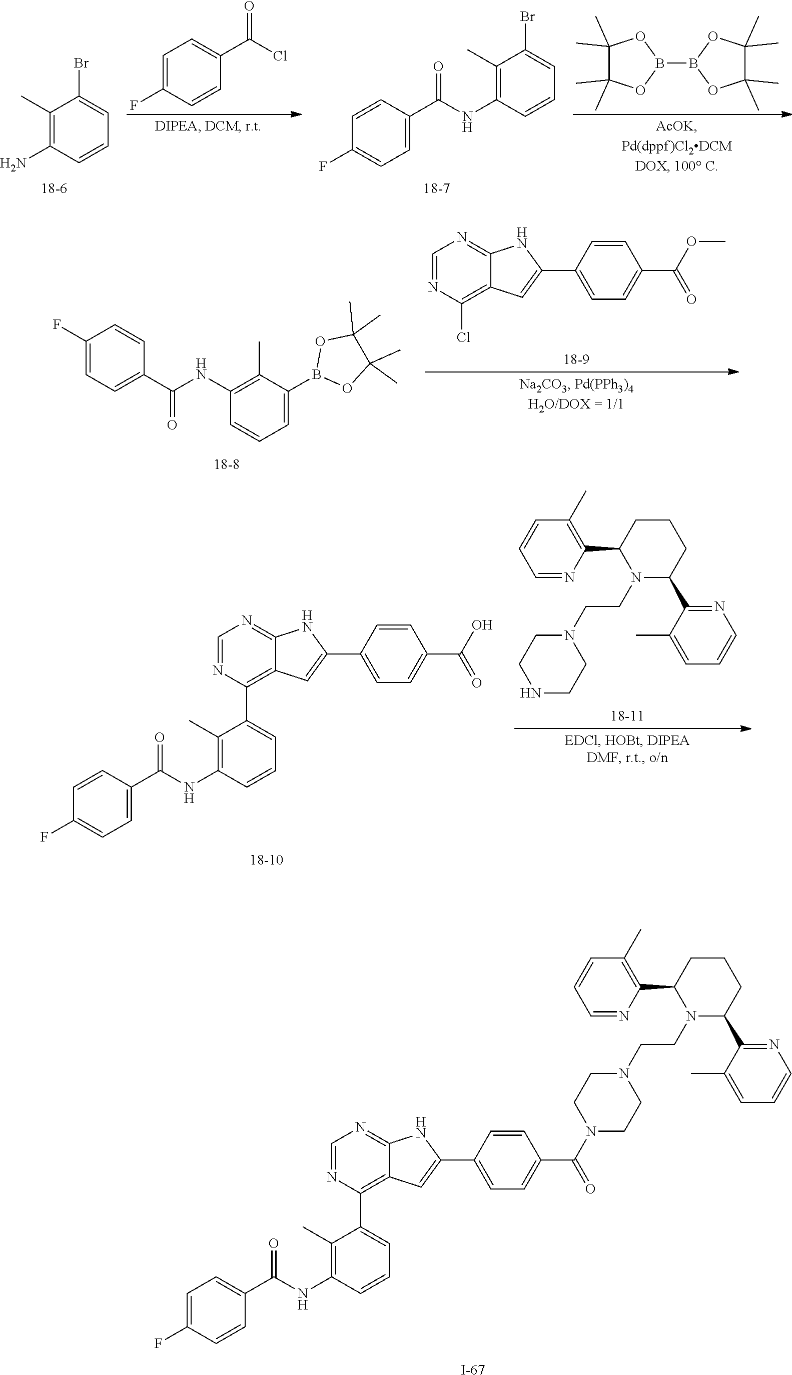

### The Synthesis of 18-7

To a solution of 3-bromo-2-methylaniline (18-6, 1.5 g, 8.108 mmol) in DCM (60 mL) was added DIPEA (2.1 g, 16.216 mmol), 4-fluorobenzoyl chloride (1.41 g, 8.919 mmol) at room temperature overnight. After completion of the reaction indicated by LCMS, the mixture was quenched with water, extracted with DCM (80 mL×3). The combined organic layers were washed with brine, dried over anhydrous Na₂SO₄, filtered and concentrated in vacuum to give a residue, which was purified by column chromatography (PE:EA=3:1) to give N-(3-bromo-2-methylphenyl)-4-fluorobenzamide (18-7, 1.9 g, 76%) as white solid.

### The Synthesis of 18-8

To a solution of N-(3-bromo-2-methylphenyl)-4-fluorobenzamide (18-7, 1.9 g, 6.189 mmol), 4,4,4′,4′,5,5,5′,5′-octamethyl-2,2′-bi (1,3,2-dioxaborolane) (1.73 g, 6.808 mmol), AcOK (1.2 g, 12.378 mmol), Pd(dppf)Cl₂·DCM (506 mg, 0.619 mmol) in 1,4-dioxane (DOX) (40 mL) under N₂ at room temperature. The mixture was stirred at 100° C. for overnight. After completion of the reaction indicated by LCMS, the mixture was filtered and concentrated in vacuum to give a residue, which was purified by column chromatography (PE:EA=3:1) to give 4-fluoro-N-(2-methyl-3-(4,4,5,5-tetramethyl-1,3,2-dioxaborolan-2-yl)phenyl)benzamide (18-8, 2.0 g, 91%) as yellow solid.

### The Synthesis of 18-10

To a solution of 4-fluoro-N-(2-methyl-3-(4,4,5,5-tetramethyl-1,3,2-dioxaborolan-2-yl)phenyl)benzamide (18-8, 350 mg, 0.986 mmol), methyl 4-(4-chloro-7H-pyrrolo[2,3-d]pyrimidin-6-yl)benzoate (18-9, 283 mg, 0.986 mmol), Na₂CO₃ (314 mg, 2.958 mmol), Pd(PPh₃)₄ (114 mg, 0.098 mmol) in DOX (2 mL) and water (2 mL) and heated in the microwave at 120° C. for 1 h. After cooling to room temperature the mixture was concentrated to give the residue, the crude material was purified by reversed column chromatography to give 4-(4-(3-(4-fluorobenzamido)-2-methylphenyl)-7H-pyrrolo[2,3-d]pyrimidin-6-yl)benzoic acid (18-10, 250 mg, 54%) as a brown solid.

### The Synthesis of I-67

To a solution of 4-(4-(3-(4-fluorobenzamido)-2-methylphenyl)-7H-pyrrolo[2,3-d]pyrimidin-6-yl)benzoic acid (18-10, 150 mg, 0.322 mmol), 1-(2-((2S,6R)-2,6-bis(3-methylpyridin-2-yl)piperidin-1-yl)ethyl)piperazine (18-11, 122 mg, 0.322 mmol), EDCI (93 mg, 0.483 mmol), DIPEA (125 mg, 0.966 mmol) and HOBT (65 mg, 0.483 mmol) in DMF (3 mL), then the reaction mixture was stirred at room temperature for overnight. After completion of the reaction indicated by LCMS, the mixture was concentrated in vacuum to give the residue, which was purified by column chromatography (DCM:MeOH=9:1) and prep-HPLC to give I-67 (58.69 mg, 22%) as white solid. LCMS (Agilent LCMS 1200-6120, Column: Waters X-Bridge C18 (50 mm*4.6 mm*3.5 μm); Column Temperature: 40° C.; Flow Rate: 2.0 mL/min; Mobile Phase: from 95% [water+10 mM NH₄HCO₃] and 5% [CH₃CN] to 0% [water+10 mM NH₄HCO₃] and 100% [CH₃CN] in 1.6 min, then under this condition for 1.4 min, finally changed to 95% [water+10 mM NH₄HCO₃] and 5% [CH₃CN] in 0.1 min and under this condition for 0.7 min.) Purity: 99.33%. Rt=1.822 min; MS Calcd.: 827.4; MS Found: 828.3 [M+H]⁺. HPLC (Agilent HPLC 1200, Column: L-column2 ODS (150 mm*4.6 mm*5.0 μm); Column Temperature: 40° C.; Flow Rate: 1.0 mL/min; Mobile Phase: from 90% [(total 10 mM AcONH₄) H₂O/MeCN=900/100 (v/v)] and 10% [total 10 mM AcONH₄) H₂O/MeCN=100/900 (v/v)] to 15% [total 10 mM AcONH₄) H₂O/MeCN=900/100 (v/v)] and 85% [total 10 mM AcONH₄) H₂O/MeCN=100/900 (v/v)] in 5 min, then under this condition for 10 min, finally changed to 90% [(total 10 mM AcONH₄) H₂O/MeCN=900/100 (v/v)] and 10% [total 10 mM AcONH₄) H₂O/MeCN=100/900 (v/v)] in 0.1 min and under this condition for 5 min. Purity: 99.66%, Rt=9.162 min; MS Calcd.: 827.4; MS Found: 829.2 [M+H]⁺. ¹H NMR (400 MHZ, DMSO-d₆): 12.81 (s, 1H), 10.08 (s, 1H), 8.87 (s, 1H), 8.35 (d, J=3.6 Hz, 2H), 8.12-8.08 (m, 2H), 7.99 (d, J=8.4 Hz, 2H), 7.55-7.51 (m, 3H), 7.47-7.34 (m, 6H), 7.19-7.16 (m, 2H), 6.87 (s, 1H), 4.29 (d, J=10.0 Hz, 2H), 3.27 (s, 2H), 3.05-3.02 (m, 2H), 2.54 (s, 6H), 2.45-2.41 (m, 2H), 2.20 (s, 5H), 2.02-1.99 (m, 1H), 1.70-1.67 (m, 1H), 1.54-1.45 (m, 6H), 0.94-0.92 (m, 2H).

### Synthesis of I-68, 1-69, and I-70

### Synthetic Scheme

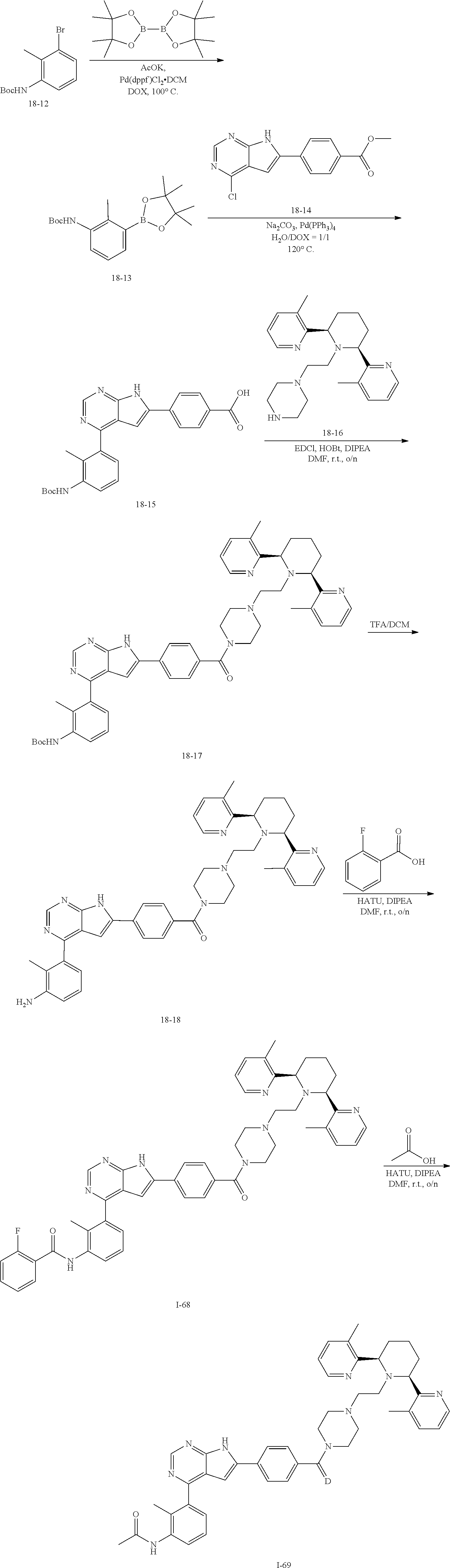

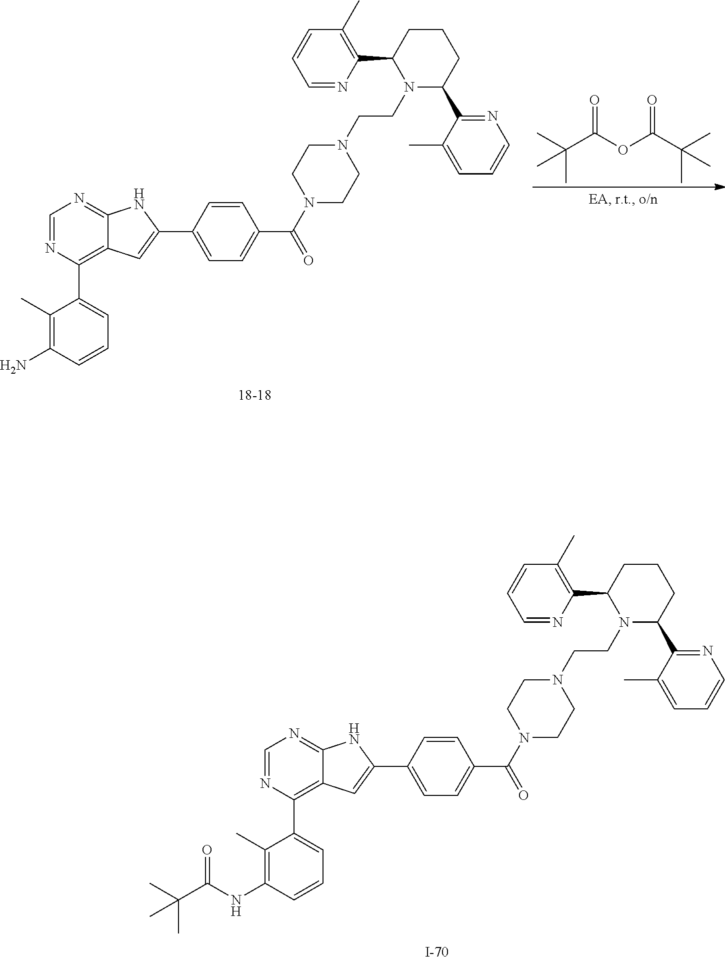

### The Synthesis of 18-13

A solution of 18-12 (2.86 g, 10 mmol), 4,4,4′,4′,5,5,5′,5′-octamethyl-2,2′-bi (1,3,2-dioxaborolane) (2.79 g, 11 mmol), AcOK (2.0 g, 20 mmol), Pd(dppf)Cl₂·DCM (816.47 mg, 1.0 mmol) in DOX (20 mL) under N₂ at room temperature was stirred at 100° C. for overnight. After completion of the reaction indicated by LCMS, the mixture was filtered and concentrated in vacuum to give a residue, which was purified by column chromatography (PE:EA=3:1) to give 18-13, 3.0 g, 90%) as white solid.

### The Synthesis of 18-15

A solution of 18-13, (1.67 g, 5.0 mmol), 18-14 (1.43 g, 5.0 mmol), Na₂CO₃ (1.06 g, 10.0 mmol), Pd(PPh₃)₄ (577 mg, 0.5 mmol) in DOX (10 mL) and water (20 mL) was heated in the microwave at 120° C. for 1 h. After cooling to room temperature the mixture was concentrated to give the residue, the crude material was purified by reversed column chromatography to get 18-15 (1.42 g, 64%) as a brown solid.

### The Synthesis of 18-17

A solution of 18-15 (1.33 g, 3.0 mmol), 18-16 (1.12 g, 3.0 mmol), EDCI (770 mg, 4.0 mmol), DIPEA (1.04 g, 8.0 mmol) and HOBT (540 mg, 4.0 mmol) in DMF (20 mL) was stirred at room temperature for overnight. After completion of the reaction indicated by LCMS, the mixture was concentrated in vacuum to give the residue, which was purified by column chromatography (DCM:MeOH=9:1) and prep-HPLC to give 18-17 (1.3 g, 54%) as white solid.

### The Synthesis of 18-18

A solution of 18-17 (806 mg, 1.0 mmol) in DCM (12 mL) and TFA (3 mL) was stirred at room temperature for overnight. After completion of the reaction indicated by LCMS, the mixture was concentrated in vacuum to give the crude. The crude was poured into water (20 mL), alkalified with sat. NaHCO₃ solution, then extracted with DCM (30 mL×4). The organic layer was dried over Na₂SO₄, filtered and concentrated in vacuum to give a residue, which was purified by column chromatography (DCM:MeOH=8:1) to give 18-18 (500 mg, 71%) as white solid.

### The Synthesis of I-68

A solution of 18-18 (71 mg, 0.1 mmol), 2-fluorobenzoic acid (70 mg, 0.5 mmol), HATU (57 mg, 0.15 mmol), DIPEA (81 mg, 0.3 mmol) in DMF (3 mL) was stirred at room temperature for overnight. After completion of the reaction indicated by LCMS, the mixture was concentrated in vacuum to give the residue, which was purified by column chromatography (DCM:MeOH=9:1) and prep-HPLC to give I-68 (24.8 mg, 30%) as white solid.

LCMS (Agilent LCMS 1200-6120, Column: Waters X-Bridge C18 (50 mm*4.6 mm*3.5 μm); Column Temperature: 40° C.; Flow Rate: 2.0 mL/min; Mobile Phase: from 95% [water+10 mM NH₄HCO₃] and 5% [CH₃CN] to 0% [water+10 mM NH₄HCO₃] and 100% [CH₃CN] in 1.6 min, then under this condition for 1.4 min, finally changed to 95% [water+10 mM NH₄HCO₃] and 5% [CH₃CN] in 0.1 min and under this condition for 0.7 min.) Purity: 99.65%. Rt=2.25 min; MS Calcd.: 827.4; MS Found: 828.2 [M+H]⁺. HPLC (Agilent HPLC 1200, Column: L-column2 ODS (150 mm*4.6 mm*5.0 μm); Column Temperature: 40° C.; Flow Rate: 1.0 mL/min; Mobile Phase: from 90% [(total 10 mM AcONH₄) H₂O/MeCN=900/100 (v/v)] and 10% [total 10 mM AcONH₄) H₂O/MeCN=100/900 (v/v)] to 15% [total 10 mM AcONH₄) H₂O/MeCN=900/100 (v/v)] and 85% [total 10 mM AcONH₄) H₂O/MeCN=100/900 (v/v)] in 5 min, then under this condition for 10 min, finally changed to 90% [(total 10 mM AcONH₄) H₂O/MeCN=900/100 (v/v)] and 10% [total 10 mM AcONH₄) H₂O/MeCN=100/900 (v/v)] in 0.1 min and under this condition for 5 min. Purity: 98.62%, Rt=10.66 min. ¹H NMR (400 MHZ, DMSO-d₆) ô: 12.81 (s, 1H), 10.00 (s, 1H), 8.56 (d, J=5.2 Hz, 1H), 8.34 (d, J=4.8 Hz, 2H), 8.00-7.99 (m, 2H), 7.77 (t, J=6.4 Hz, 1H), 7.64-7.62 (m, 4H), 7.57-7.33 (m, 6H), 6.19-7.16 (m, 2H), 6.88 (s, 1H), 4.27 (d, J=11.2 Hz, 2H), 3.27-3.15 (m, 2H), 3.11-3.02 (m, 2H), 2.51 (s, 6H), 2.50-2.49 (m, 2H), 2.42-2.34 (m, 5H), 2.07-2.01 (m, 1H), 1.78-1.58 (m, 7H), 0.82 (br, 2H).

### The Synthesis of I-69

A solution of 18-18 (71 mg, 0.1 mmol), AcOH (35 mg, 0.5 mmol), HATU (57 mg, 0.15 mmol), DIPEA (81 mg, 0.3 mmol) in DMF (3 mL) was stirred at room temperature for overnight. After completion of the reaction indicated by LCMS, the mixture was concentrated in vacuum to give the residue, which was purified by column chromatography (DCM:MeOH=9:1) and prep-HPLC to give I-69 (26.2 mg, 35%) as white solid.

LCMS (Agilent LCMS 1200-6120, Column: Waters X-Bridge C18 (50 mm*4.6 mm*3.5 μm); Column Temperature: 40° C.; Flow Rate: 2.0 mL/min; Mobile Phase: from 95% [water+10 mM NH₄HCO₃] and 5% [CH₃CN] to 0% [water+10 mM NH₄HCO₃] and 100% [CH₃CN] in 1.6 min, then under this condition for 1.4 min, finally changed to 95% [water+10 mM NH₄HCO₃] and 5% [CH₃CN] in 0.1 min and under this condition for 0.7 min.) Purity: 97.48%. Rt=2.14 min; MS Calcd.: 747.4; MS Found: 748.2 [M+H]⁺. HPLC (Agilent HPLC 1200, Column: L-column2 ODS (150 mm*4.6 mm*5.0 μm); Column Temperature: 40° C.; Flow Rate: 1.0 mL/min; Mobile Phase: from 90% [(total 10 mM AcONH₄) H₂O/MeCN=900/100 (v/v)] and 10% [total 10 mM AcONH₄) H₂O/MeCN=100/900 (v/v)] to 15% [total 10 mM AcONH₄) H₂O/MeCN=900/100 (v/v)] and 85% [total 10 mM AcONH₄) H₂O/MeCN=100/900 (v/v)] in 5 min, then under this condition for 10 min, finally changed to 90% [(total 10 mM AcONH₄) H₂O/MeCN=900/100 (v/v)] and 10% [total 10 mM AcONH₄) H₂O/MeCN=100/900 (v/v)] in 0.1 min and under this condition for 5 min. Purity: 100.00%, Rt=8.00 min. ¹H NMR (400 MHZ, DMSO-d₆): 12.78 (s, 1H), 9.46 (s, 1H), 8.78 (s, 1H), 8.34 (d, J=4.0 Hz, 2H), 7.98 (d, J=4.0 Hz, 2H), 7.55-7.53 (m, 3H), 7.37-7.33 (m, 4H), 6.19-7.16 (m, 2H), 6.86 (s, 1H), 4.27 (d, J=11.2 Hz, 2H), 3.27-3.15 (m, 2H), 3.11-3.02 (m, 2H), 2.51 (s, 6H), 2.50-2.49 (m, 2H), 2.42-2.34 (m, 5H), 2.24 (s, 3H), 2.07-2.01 (m, 1H), 1.78-1.58 (m, 7H), 0.82 (br, 2H).

### The Synthesis of I-70

A solution of 18-18 (71 mg, 0.1 mmol), pivalic anhydride (100 mg, 0.5 mmol) in EA (3 mL) was stirred at room temperature for overnight. After completion of the reaction indicated by LCMS, the mixture was concentrated in vacuum to give the residue, which was purified by column chromatography (DCM:MeOH=9:1) and prep-HPLC to give I-70 (30.8 mg, 39%) as white solid. LCMS (Agilent LCMS 1200-6120, Column: Waters X-Bridge C18 (50 mm*4.6 mm*3.5 μm); Column Temperature: 40° C.; Flow Rate: 2.0 mL/min; Mobile Phase: from 95% [water+10 mM NH₄HCO₃] and 5% [CH₃CN] to 0% [water+10 mM NH₄HCO₃] and 100% [CH₃CN] in 1.6 min, then under this condition for 1.4 min, finally changed to 95% [water+10 mM NH₄HCO₃] and 5% [CH₃CN] in 0.1 min and under this condition for 0.7 min.) Purity: 99.74%. Rt=1.83 min; MS Calcd.: 789.5; MS Found: 790.4 [M+H]⁺. HPLC (Agilent HPLC 1200, Column: L-column2 ODS (150 mm*4.6 mm*5.0 μm); Column Temperature: 40° C.; Flow Rate: 1.0 mL/min; Mobile Phase: from 90% [(total 10 mM AcONH₄) H₂O/MeCN=900/100 (v/v)] and 10% [total 10 mM AcONH₄) H₂O/MeCN=100/900 (v/v)] to 15% [total 10 mM AcONH₄) H₂O/MeCN=900/100 (v/v)] and 85% [total 10 mM AcONH₄) H₂O/MeCN=100/900 (v/v)] in 5 min, then under this condition for 10 min, finally changed to 90% [(total 10 mM AcONH₄) H₂O/MeCN=900/100 (v/v)] and 10% [total 10 mM AcONH₄) H₂O/MeCN=100/900 (v/v)] in 0.1 min and under this condition for 5 min. Purity: 99.76%, Rt=9.75 min. ¹H NMR (400 MHZ, DMSO-d₆) δ: 12.78 (s, 1H), 9.46 (s, 1H), 8.78 (s, 1H), 8.34 (d, J=4.0 Hz, 2H), 7.98 (d, J=4.0 Hz, 2H), 7.55-7.53 (m, 3H), 7.37-7.33 (m, 4H), 6.19-7.16 (m, 2H), 6.86 (s, 1H), 4.27 (d, J=11.2 Hz, 2H), 3.27-3.15 (m, 2H), 3.11-3.02 (m, 2H), 2.51 (s, 6H), 2.50-2.49 (m, 2H), 2.42-2.34 (m, 5H), 2.07-2.01 (m, 1H), 1.78-1.58 (m, 7H), 1.27 (s, 9H), 0.82 (br, 2H).

Additional, exemplary compounds of the present invention were prepared using methods similar to those described above and/or using methods known in the art. Their structures and characterization data are shown below.

| Com- | | | |
|---|---|---|---|
| pound | | | |
| # | | LCMS | ¹H NMR |
| I-20 | 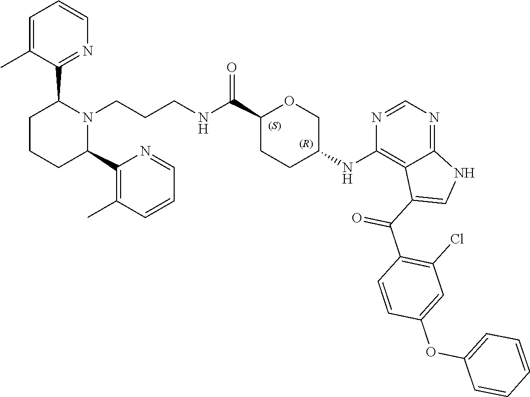 | LCMS (Agilent LCMS 1200-6120, Column: Waters X-Bridge C18 (50 mm*4.6 mm*3.5 μm); Column Temperature: 40° C.; Flow Rate: 2.0 mL/min; Mobile Phase: from 95% [water + 10 mM NH₄HCO₃] and 5% [CH₃CN] to 0% [water + 10 mM NH₄HCO₃] and 100% [CH₃CN] in 1.6 min, then under this condition for 1.4 min, finally changed to 95% [water + 10 mM NH₄HCO₃] and 5% [CH₃CN] in 0.1 min and under this condition for 0.7 min.) Purity: 100.00%. Rt = 2.001 min; MS Calcd.: 798.3; MS Found: | ¹H NMR (400 MHz, DMSO-d₆) δ: 12.5 (s, 1H), 8.61 (d, J = 7.2 Hz, 1H), 8.36 (s, 2H), 8.28 (s, 1H), 7.66 (s, 1H), 7.59- 7.50 (m, 3H), 7.48-7.46 (m, 2H), 7.26 (t, J = 7.2 Hz, 1H), 7.20-7.18 (m, 5H), 7.02 (dd, J = 2.4, 8.4 Hz, 1H), 6.89 (s, 1H), 4.18- 4.12 (m, 4H), 3.57 (d, J = 11.2 Hz, 1H), 3.16 (t, J = 10.0 Hz, 1H), 2.55 (s, 6H), 2.41-2.29 (m, 3H), 2.24-2.11 (m, 3H), 1.99-1.88 (m, 2H), 1.65-1.56 (m, 3H), 1.52-1.50 (m, 2H), 1.23-1.18 |
| | | 799.2 [M + H]⁺. | (m, 1H), 0.53-0.48 |
| | | | (m, 2H). |
| I-21 | 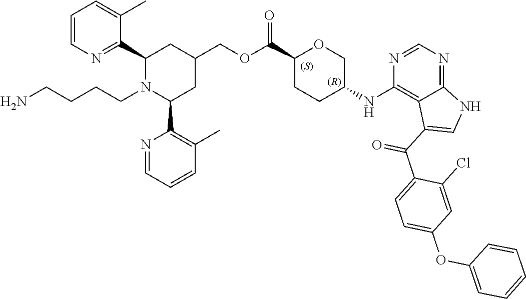 | LCMS (Agilent LCMS 1200-6120, Column: Waters X-Bridge C18 (50 mm*4.6 mm*3.5 μm); Column Temperature: 40° C.; Flow Rate: 2.0 mL/min; Mobile Phase: from 95% [water + 10 mM NH₄HCO₃] and 5% [CH₃CN] to 0% [water + 10 mM NH₄HCO₃] and 100% [CH₃CN] in 1.6 min, then under this condition for 1.4 min, finally changed to 95% [water + 10 mM NH₄HCO₃] and 5% [CH₃CN] in 0.1 min and | ¹H NMR (400 MHz, DMSO-d₆): δ 9.61 (s, 2H), 8.92 (d, J = 7.6 Hz, 1H), 8.65 (dd, J = 4.0, 13.6 Hz, 2H), 8.50 (s, 1H), 8.30 (s, 1H), 7.55 (d, J = 7.6 Hz, 2H), 7.46- 7.40 (m, 2H), 7.37 (d, J = 8.4 Hz, 2H), 7.25-7.19 (m, 3H), 7.12-7.07 (m, 3H), 6.93 (dd, J = 2.4, 6.0 Hz, 1H), 4.40 (dd, J = 9.6, 12.4 Hz, 2H), 4.10-3.98 (m, 3H), 3.86 (d, J = 10.4 |
| | | under this condition for | Hz, 2H), 3.36 (t, |
| | | 0.7 min.) Purity: 100.00 | J = 11.2 Hz, 1H), |
| | | %. Rt = 1.065 min; MS | 3.02-2.93 (m, 2H), |
| | | Calcd.: 842.4; MS | 2.43 (s, 6H), 2.33- |
| | | Found: 422.4 [M/2 + H]⁺. | 2.66 (m, 3H), 2.15- |
| | | | 2.11 (m, 2H), 1.85- |
| | | | 1.63 (m, 6H), 1.01 |
| | | | (, J = 5.6 Hz, 2H), |
| | | | 0.45 (d, J = 4.4 Hz, |
| | | | 2H). |
| I-22 | 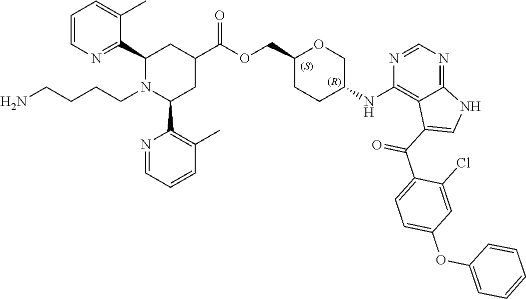 | LCMS (Agilent LCMS 1200-6120, Column: Waters X-Bridge C18 (50 mm*4.6 mm*3.5 μm); Column Temperature: 40° C.; Flow Rate: 2.0 mL/min; Mobile Phase: from 95% [water + 10 mM NH₄HCO₃] and 5% [CH₃CN] to 0% [water + 10 mM NH₄HCO₃] and 100% [CH₃CN] in 1.6 min, then under this condition for 1.4 min, finally changed to 95% [water + 10 mM NH₄HCO₃] and 5% [CH₃CN] in 0.1 min | 1H NMR (400 MHz, DMSO-d₆): δ 9.74-9.55 (m, 2H), 8.87 (t, J = 7.2 Hz, 1H), 8.64- 8.58 (m, 2H), 8.50-8.43 (m, 1H), 8.29 (d, J = 11.6 Hz, 1H), 7.53 (q, J = 8.0 Hz, 2H), 7.45-7.37 (m, 4H), 7.25-7.17 (m, 3H), 7.12-7.07 (m, 3H), 6.97-6.94 (m, 1H), 4.39-4.22 (m, 2H), 4.17-3.98 (m, 2H), 3.88 (dd, J = 6.8, 13.2 Hz, 2H), 3.29 (t, J = 11.6 Hz, |
| | | and under this | 1H), 2.96-2.90 (m, |
| | | condition for 0.7 min.) | 3H), 2.75-2.67 (m, |
| | | Purity: 100.00%. Rt = | 1H), 2.44 (s, 6H), |
| | | 1.071 min; MS Calcd.: | 2.34-2.22 (m, 3H), |
| | | 842.4; MS Found: | 2.15 (d, J = 14.0 |
| | | 843.3 [M + H]⁺. | Hz, 1H), 2.09- |
| | | | 2.01 (m, 1H), |
| | | | 1.85-1.51 (m, 3H), |
| | | | 1.00 (s, 2H), 0.46 |
| | | | (m, 2H). |
| I-26 | 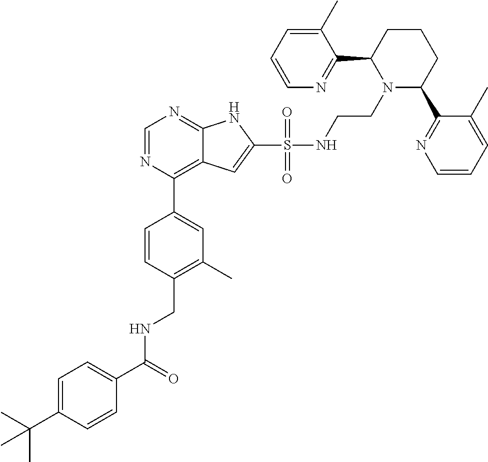 [SMILES: Cc1cc(-c2ncnc3[nH]c(S(=O)(=O)NCCN4[C@@H](c5ncccc5C)CCC[C@H]4c4ncccc4C)cc23)ccc1CNC(=O)c1ccc(C(C)(C)C)cc1] | LCMS (Agilent LCMS 1200-6120, Column: Waters X-Bridge C18 (50 mm*4.6 mm*3.5 μm); Column Temperature: 40° C.; Flow Rate: 2.0 mL/min; Mobile Phase: from 95% [water + 10 mM NH₄HCO₃] and 5% [CH₃CN] to 0% [water + 10 mM NH₄HCO₃] and 100% [CH₃CN] in 1.6 min, then under this condition for 1.4 min, finally changed to 95% [water + 10 mM NH₄HCO₃] and 5% [CH₃CN] in 0.1 min and under this condition for 0.7 min.) Purity: 97.64%. Rt = 2.063 min; MS Calcd.: 770.4; MS Found: 771.4 [M + H]⁺. | ¹H NMR (400 MHz, DMSO-d₆) δ: 12.98 (s, 1H), 9.02 (q, J = 5.6 Hz, 2H), 8.08 (s, 2H), 7.98 (t, J = 6.4 Hz, 2H), 7.89 (d, J = 8.4 Hz, 2H), 7.51 (d, J = 7.2 Hz, 3H), 7.22 (d, J = 7.6 Hz, 2H), 7.03 (t, J = 1.6 Hz, 1H), 6.87 (s, 1H), 6.77 (s, 2H), 4.59 (d, J = 6.0 Hz, 2H), 4.10 (t, J = 8.0 Hz, 2H), 2.51 (d, J = 2.4 Hz, 3H), 2.43- 2.28 (m, 8H), 1.99- 1.92 (m, 2H), 1.85 (t, J = 10.4 Hz, 1H), 1.67-1.56 (m, 3H), 1.43 (d, J = 12.8 Hz, 2H), 1.31 (s, 9H). |
| I-27 | 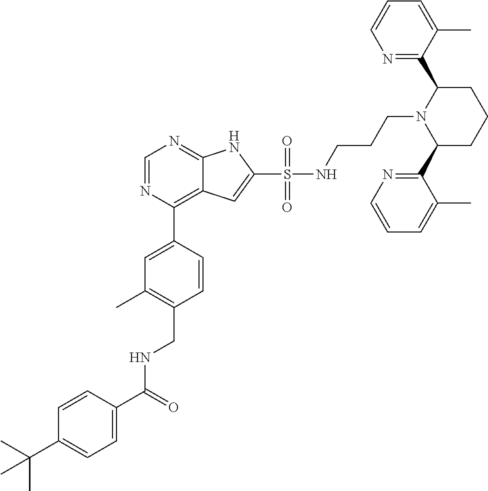 [SMILES: Cc1cc(-c2ncnc3[nH]c(S(=O)(=O)NCCCN4[C@@H](c5ncccc5C)CCC[C@H]4c4ncccc4C)cc23)ccc1CNC(=O)c1ccc(C(C)(C)C)cc1] | LCMS (Agilent LCMS 1200-6120, Column: Waters X-Bridge C18 (50 mm*4.6 mm*3.5 μm); Column Temperature: 40 °C.; Flow Rate: 2.0 mL/min; Mobile Phase: from 95% [water + 10 mM NH₄HCO₃] and 5% [CH₃CN] to 0% [water + 10 mM NH₄HCO₃] and 100% [CH₃CN] in 1.6 min, then under this condition for 1.4 min, finally changed to 95% [water + 10 mM NH₄HCO₃] and 5% [CH₃CN] in 0.1 min and under this condition for 0.7 min.) Purity: 97.53 %. Rt = 2.017 min; MS Calcd.: 784.4; MS Found: 785.5 [M + H]⁺. | ¹H NMR (400 MHz, DMSO-d₆) δ: 13.04 (s, 1H), 9.02 (t, J = 5.6 Hz, 2H), 8.18 (d, J = 2.8 Hz, 2H), 7.98 (t, J = 6.8 Hz, 2H), 7.88 (d, J = 8.4 Hz, 2H), 7.51 (d, J = 8.8 Hz, 3H), 7.24 (s, 1H), 7.13 (d, J = 8.2 Hz, 2H), 7.04 (s, 1H), 6.85 (s, 2H), 4.58 (d, J = 5.6 Hz, 2H), 4.11 (t, J = 5.6 Hz, 2H), 2.51-2.50 (m, 3H), 2.41-2.28 (m, 8H), 2.14-2.03 (m, 2H), 1.89 (t, J = 9.2 Hz, 1H), 1.76 (d, J = 13.2 Hz, 2H), 1.58 (q, J = 7.2 Hz, 1H), 1.46-1.34 (m, 2H), 1.31 (s, 9H), 0.25- 0.22 (m, 2H). |
| I-28 | 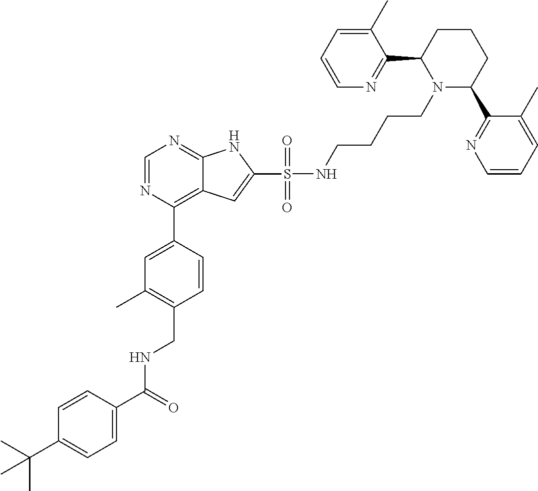 [SMILES: Cc1cc(-c2ncnc3[nH]c(S(=O)(=O)NCCCCN4[C@@H](c5ncccc5C)CCC[C@H]4c4ncccc4C)cc23)ccc1CNC(=O)c1ccc(C(C)(C)C)cc1] | LCMS (Agilent LCMS 1200-6120, Column: Waters X-Bridge C18 (50 mm*4.6 mm*3.5 μm); Column Temperature: 40° C.; Flow Rate: 2.0 mL/min; Mobile Phase: from 95% [water + 10 mM NH₄HCO₃] and 5% [CH₃CN] to 0% [water + 10 mM NH₄HCO₃] and 100% [CH₃CN] in 1.6 min, then under this condition for 1.4 min, finally changed to 95% [water + 10 mM NH₄HCO₃] and 5% [CH₃CN] in 0.1 min and under this condition for 0.7 min.) Purity: 100.00 %. Rt = 2.024 min; MS Calcd.: 798.4; MS Found: 799.4 [M + H]⁺. | ¹H NMR (400 MHz, DMSO-d₆) δ: 13.13 (s, 1H), 9.01 (t, J = 6.0 Hz, 2H), 8.19 (s, 2H), 7.97 (d, J = 8.8 Hz, 2H), 7.88 (d, J = 8.4 Hz, 2H), 7.52-7.42 (m, 4H), 7.37 (d, J = 7.2 Hz, 2H), 7.15 (s, 1H), 6.97 (q, J = 4.8 Hz, 2H), 4.56 (d, J = 5.6 Hz, 2H), 4.11 (s, 2H), 2.49 (s, 3H), 2.43 (s, 6H), 2.13- 1.97 (m, 6H), 1.88 (t, J = 12.4 Hz, 1H), 1.59-1.51 (m, 1H), 1.50-1.38 (m, 2H), 1.31 (s, 9H), 0.56 (s, 2H), 0.15- 0.10 (m, 2H). |
| I-29 | 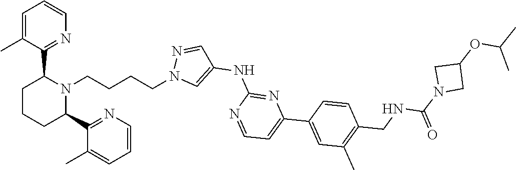 | LCMS (Agilent LCMS 1200-6120, Column: Waters X-Bridge C18 (50 mm*4.6 mm*3.5 μm); Column Temperature: 40° C.; Flow Rate: 2.0 mL/min; Mobile Phasc: from 95% [water + 10 mM NH₄HCO₃] and 5% [CH₃CN] to 0% [water + | ¹H NMR (400 MHz, DMSO-d₆) δ: 8.43 (d, J = 5.6 Hz, 3H), 7.79 (d, J = 8.0 Hz, 2H), 7.72-7.63 (m, 1H), 7.49 (s, 1H), 7.39 (s, 3H), 7.14-7.06 (m, 3H), 4.46 (d, J = 5.2 Hz, 2H), 4.38-4.33 (m, 1H), |
| | | 10 mM NH₄HCO₃] | 4.17 (s, 2H), 4.05- |
| | | and 100% [CH₃CN] in | 3.96 (m, 1H), |
| | | 1.6 min, then under this | 3.90-3.87 (m, 2H), |
| | | condition for 1.4 min, | 3.75-3.59 (m, 3H), |
| | | finally changed to 95% | 2.65-2.53 (m, 1H), |
| | | [water + 10 mM | 2.42 (s, 6H), 2.26- |
| | | NH₄HCO₃] and 5% | 2.20 (m, 1H), |
| | | [CH₃CN] in 0.1 min | 2.11-2.05 (m, 2H), |
| | | and under this | 2.05 (s, 3H), 1.79- |
| | | condition for 0.7 min.) | 1.70 (m, 2H), |
| | | Purity: 100.00%. Rt = | 1.67-1.52 (m, 2H), |
| | | 1.825 min; MS Calcd.: | 1.31-1.26 (m, 3H), |
| | | 742.1; MS Found: | 1.15 (d, J = 6.0 |
| | | 743.4 [M + H]⁺. | Hz, 6H), 0.99- |
| | | | 0.81 (m, 2H). |
| I-30 | 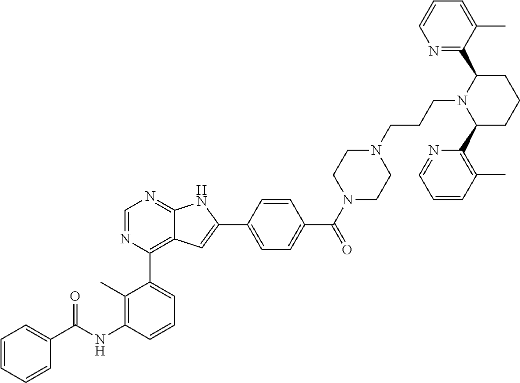 [SMILES: Cc1cccnc1[C@H]1CCC[C@@H](c2ncccc2C)N1CCCN1CCN(C(=O)c2ccc(-c3cc4c(-c5cccc(NC(=O)c6ccccc6)c5C)ncnc4[nH]3)cc2)CC1] | LCMS (Agilent LCMS 1200-6120, Column: Waters X-Bridge C18 (50 mm*4.6 mm*3.5 μm); Column Temperature: 40° C.; Flow Rate: 2.0 mL/min; Mobile Phase: from 95% [water + 10 mM NH₄HCO₃] and 5% [CH₃CN] to 0% [water + 10 mM NH₄HCO₃] and 100% [CH₃CN] in 1.6 min, then under this condition for 1.4 min, finally changed to 95% [water + 10 mM NH₄HCO₃] and 5% [CH₃CN] in 0.1 min and under this condition for 0.7 min.) Purity: 100%. Rt = 1.833 min; MS Calcd.: 823.4; MS Found: 824.3 [M + H]⁺. | ¹H NMR (400 MHz, DMSO-d₆) δ: 12.83 (s, 1H), 10.05 (s, 1H), 8.87 (s, 1H), 8.23 (s, 2H), 8.06-7.92 (m, 4H), 7.63-7.49 (m, 6H), 7.45-7.39 (m, 4H), 7.17-7.14 (m, 2H), 6.88 (s, 1H), 4.22-4.19 (m, 1H), 3.26-3.20 (m, 2H), 3.19-3.04 (m, 2H), 2.67 (s, 6H), 2.54- 2.50 (m, 3H), 2.23 (s, 3H), 2.19-2.09 (m, 2H), 1.98-1.91 (m, 1H), 1.88-1.65 (m, 4H), 1.61-1.54 (m, 1H), 1.51-1.38 (m, 3H), 0.49-0.38 (m, 2H). |
| I-31 | 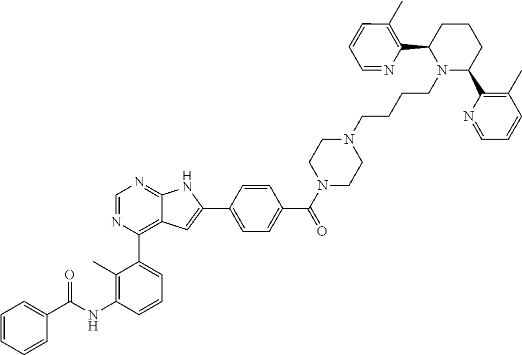 | LCMS (Agilent LCMS 1200-6120, Column: Waters X-Bridge C18 (50 mm*4.6 mm*3.5 μm); Column Temperature: 40° C.; Flow Rate: 2.0 mL/min; Mobile Phase: from 95% [water + 10 mM NH₄HCO₃] and 5% [CH₃CN] to 0% [water + 10 mM NH₄HCO₃] and 100% [CH₃CN] in 1.6 min, then under this condition for 1.4 min, finally changed to 95% [water + 10 mM NH₄HCO₃] and 5% [CH₃CN] in 0.1 min and under this condition for 0.7 min.) Purity: 100.00%. Rt = | ¹H NMR (400 MHz, DMSO-d₆) δ: 12.83 (s, 1H), 10.05 (s, 1H), 8.87 (s, 1H), 8.34 (s, 2H), 8.05-8.02 (m, 4H), 7.63-7.51 (m, 6H), 7.47-7.40 (m, 4H), 7.17-7.14 (m, 2H), 6.89 (s, 1H), 4.22-4.18 (m, 2H), 3.48-3.40 (m, 2H), 3.28-3.18 (m, 2H), 2.54 (s, 6H), 2.33- 2.24 (m, 3H), 2.21-2.13 (m, 2H), 2.08-1.91 (m, 7H), 1.70-1.60 (m, 3H), 1.48-1.46 (m, 2H), 0.50 (s, 2H), 0.21- 0.15 (m, 2H). |
| | | 1.857 min; MS Calcd.: | |
| | | 837.4; MS Found: | |
| | | 838.3 [M + H]⁺. | |
| I-32 | 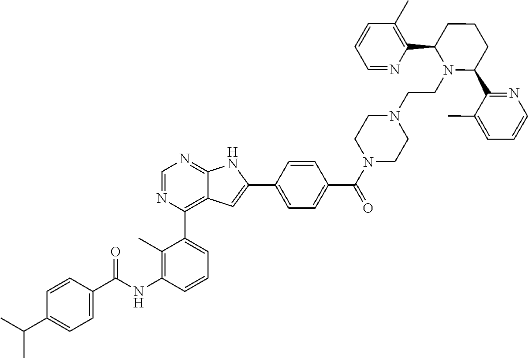 | LCMS (Agilent LCMS 1200-6120, Column: Waters X-Bridge C18 (50 mm*4.6 mm*3.5 μm); Column Temperature: 40° C.; Flow Rate: 2.0 mL/min; Mobile Phase: from 95% [water + 10 mM NH₄HCO₃] and 5% [CH₃CN] to 0% [water + 10 mM NH₄HCO₃] and 100% [CH₃CN] in 1.6 min, then under this condition for 1.4 min, finally changed to 95% [water + 10 mM NH₄HCO₃] and 5% [CH₃CN] in 0.1 min and under this condition for 0.7 min.) Purity: 97.18%. Rt = | ¹H NMR (400 MHz, DMSO-d₆) δ: 12.82 (s, 1H), 9.96 (s, 1H), 8.87 (s, 1H), 8.35 (d, J = 3.2 Hz, 2H), 7.97 (q, J = 8.4 Hz, 4H), 7.55- 7.52 (m, 3H), 7.45-7.41 (m, 4H), 7.35 (d, J = 8 Hz, 2H), 7.18 (dd, J₁ = 8.4 Hz, J₂ = 7.6 Hz, 2H), 6.87 (s, 1H), 4.29 (d, J = 12 Hz, 2H), 3.25 (s, 2H), 3.02-2.97 (m, 3H), 2.54 (s, 6H), 2.43 (s, 2H), 2.19 (s, 5H), 2.03- 1.99 (m, 1H), 1.73-1.64 (m, 1H), |
| | | 1.971 min; MS | 1.53-1.45 (m, 6H), |
| | | Calcd.: 851.5; MS | 1.25 (s, 3H), 1.24 |
| | | Found: 852.5 [M + H]⁺. | (s, 3H), 0.92 (s, |
| | | | 2H). |
| I-33 | 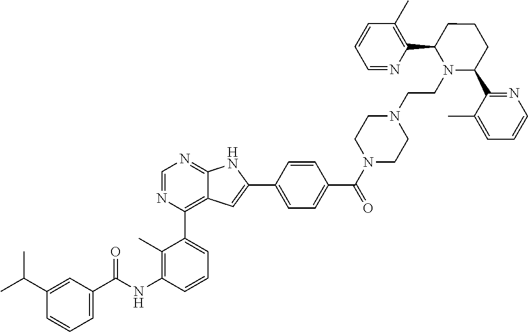 | LCMS (Agilent LCMS 1200-6120, Column: Waters X-Bridge C18 (50 mm*4.6 mm*3.5 μm); Column Temperature: 40° C.; Flow Rate: 2.0 mL/min; Mobile Phase: from 95% [water + 10 mM NH₄HCO₃] and 5% [CH₃CN] to 0% [water + 10 mM NH₄HCO₃] and 100% [CH₃CN] in 1.6 min, then under this condition for 1.4 min, finally changed to 95% [water + 10 mM NH₄HCO₃] and 5% [CH₃CN] in 0.1 min and under this condition for | ¹H NMR (400 MHz, DMSO-d₆) δ: 12.83 (s, 1H), 10.03 (s 1H), 8.87 (s, 1H), 8.35 (d, J = 3.2 Hz, 2H), 7.89 (s, 2H), 7.84 (d, J = 7.2 Hz, 2H), 7.55-7.42 (m, 7H), 7.35 (d, J = 9.4 Hz, 2H), 7.17 (dd, J = 4.4, 7.2 Hz, 2H), 6.88 (s, 1H), 4.29 (d, J = 10.4 Hz, 2H), 3.31- 3.20 (m, 2H), 3.03- 2.96 (m, 3H), 2.54 (s, 6H), 2.40 (d, J = 6.8 Hz, 2H), 2.20- 2.07 (m, 5H), 2.01- |
| | | 0.7 min.) Purity: | 1.95 (m, 1H),1.74- |
| | | 99.78%. Rt = 1.980 | 1.62 (m, 1H), 1.54- |
| | | min; MS Calcd.: 851.5; | 1.41 (m, 6H), 1.26 |
| | | MS Found: 852.5 | (d, J = 6.8 Hz, 6H), |
| | | [M + H]⁺. | 0.97-0.85 (m, 2H). |
| I-34 | 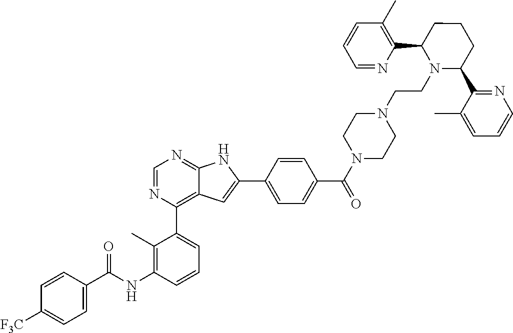 | LCMS (Agilent LCMS 1200-6120, Column: Waters X-Bridge C18 (50 mm*4.6 mm*3.5 μm); Column Temperature: 40° C.; Flow Rate: 2.0 mL/min; Mobile Phase: from 95% [water + 10 mM NH₄HCO₃] and 5% [CH₃CN] to 0% [water + 10 mM NH₄HCO₃] and 100% [CH₃CN] in 1.6 min, then under this condition for 1.4 min, finally changed to 95% [water + 10 mM NH₄HCO₃] and 5% [CH₃CN] in 0.1 min and under this condition for | ¹H NMR (400 MHz, DMSO-d₆) δ: 12.82 (s, 1H), 10.03 (s, 1H), 8.88 (s, 1H), 8.35 (d, J = 4.0 Hz, 2H), 8.22 (d, J = 8.4 Hz, 2H), 8.00 (d, J = 8.0 Hz, 2H), 7.95 (d, J = 8.0 Hz, 2H), 7.54 (d, J = 6.4 Hz, 3H), 7.49-7.44 (m, 2H), 7.35 (d, J = 8.0 Hz, 2H), 7.18 (q, J = 4.8 Hz, 2H), 6.89 (s, 1H), 4.29 (d, J = 10.8 Hz, 2H), 3.27 (d, J = 3.2 Hz, 2H), 3.02 (d, J = 1.6 Hz, 2H), 2.54 (s, 6H), |
| | | 0.7 min.) Purity: 98.98 | 2.41 (d, J = 2.8 Hz, |
| | | %. Rt = 1.923 min; MS | 2H), 2.21-2.15 (m, |
| | | Calcd.: 877.4; MS | 5H), 2.01 (d, J = |
| | | Found: 878.4 [M + H]⁺. | 7.2 Hz, 1H), 1.70 |
| | | | (s, 1H), 1.53-1.45 |
| | | | (m, 6H), 0.93-0.90 |
| | | | (m, 2H). |
| I-35 | 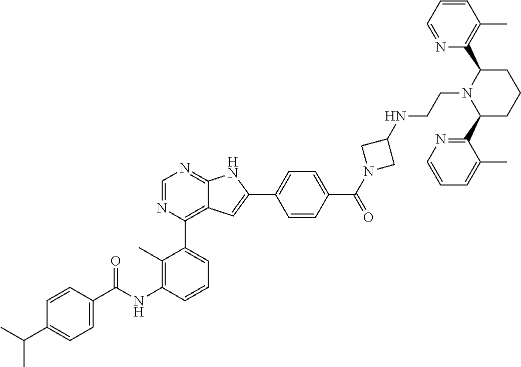 [SMILES: Cc1cccnc1[C@H]1CCC[C@@H](c2ncccc2C)N1CCNC1CN(C(=O)c2ccc(-c3cc4c(-c5cccc(NC(=O)c6ccc(C(C)C)cc6)c5C)ncnc4[nH]3)cc2)C1] | LCMS (Agilent LCMS 1200-6120, Column: Waters X-Bridge C18 (50 mm*4.6 mm*3.5 μm); Column Temperature: 40° C.; Flow Rate: 2.0 mL/min; Mobile Phase: from 95% [water + 10 mM NH₄HCO₃] and 5% [CH₃CN] to 0% [water + 10 mM NH₄HCO₃] and 100% [CH₃CN] in 1.6 min, then under this condition for 1.4 min, finally changed to 95% [water + 10 mM NH₄HCO₃] and 5% [CH₃CN] in 0.1 min and under this condition for 0.7 min.) Purity: 97.88 %. Rt = 1.912 min; MS Calcd.: 837.5; MS | ¹H NMR (400 MHz, DMSO-d₆) δ: 12.87 (s, 1H), 9.96 (s 1H), 8.88 (s, 1H), 8.35 (s, 2H), 8.05 (d, J = 9.2 Hz, 2H), 7.96 (d, J = 8.4 Hz, 2H), 7.60 (d, J = 8.4 Hz, 2H), 7.55-7.46 (m, 3H), 7.45-7.40 (m, 5H), 7.18-7.15 (m, 2H), 6.93 (s, 1H), 4.26-4.24 (m, 2H), 3.99 (d, J = 7.2 Hz, 1H), 3.76 (t, J = 9.2 Hz, 1H), 3.28- 3.19 (m, 1H), 3.02- 2.95 (m, 1H), 2.74- 2.67 (m, 1H), 2.51 (s, 6H), 2.39-2.27 (m, 2H), 2.23 (s, 5H), 1.97-1.94 (m, |
| | | Found: 838.4 [M + H]⁺. | 1H), 1.68-1.64 (m, |
| | | | 1H), 1.47 (d, J = |
| | | | 12.4 Hz, 2H), |
| | | | 1.41-1.35 (m, 1H), |
| | | | 1.24 (d, J = 7.2 Hz, |
| | | | 6H), 1.18-1.07 (m, |
| | | | 2H). |
| I-36 | 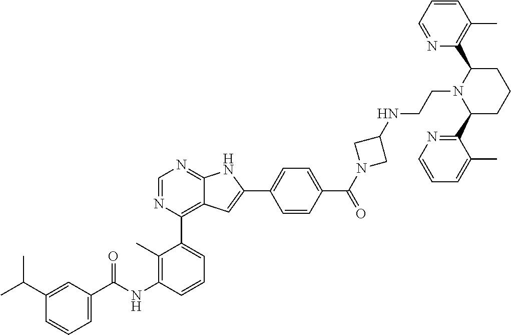 | LCMS (Agilent LCMS 1200-6120, Column: Waters X-Bridge C18 (50 mm*4.6 mm*3.5 μm); Column Temperature: 40° C.; Flow Rate: 2.0 mL/min; Mobile Phase: from 95% [water + 10 mM NH₄HCO₃] and 5% [CH₃CN] to 0% [water + 10 mM NH₄HCO₃] and 100% [CH₃CN] in 1.6 min, then under this condition for 1.4 min, finally changed to 95% [water + 10 mM NH₄HCO₃] and 5% [CH₃CN] in 0.1 min and under this condition for 0.7 min.) Purity: 97.61 | ¹H NMR (400 MHz, DMSO-d₆) δ: 12.87 (s, 1H), 10.03 (s 1H), 8.87 (s, 1H), 8.35 (d, J = 3.2 Hz, 2H), 7.89-7.62 (m, 2H), 7.61 (d, J = 8.4 Hz, 2H), 7.55-7.41 (m, 8H), 7.16 (q, J = 4.8 Hz, 2H), 6.94 (s, 1H), 4.26 (d, J = 1.6 Hz, 2H), 4.01 (d, J = 7.6 Hz, 1H), 3.76 (t, J = 8.0 Hz, 2H), 3.19 (t, J = 10.8 Hz, 1H), 3.01- 2.96 (m, 1H), 2.74 (s, 1H), 2.50 (s, 6H), 2.39-2.33 (m, |
| | | %. Rt = 1.909 min; MS | 2H), 2.20-2.08 (m, |
| | | Calcd.: 837.5; MS | 5H), 1.98 (d, J = |
| | | Found: 838.5 [M + H]⁺. | 8.8 Hz, 1H), 1.69- |
| | | | 1.65 (m, 1H), 1.49- |
| | | | 1.35 (m, 3H), 1.26 |
| | | | (d, J = 6.8 Hz, 6H), |
| | | | 1.20-1.10 (m, 2H). |
| I-37 | 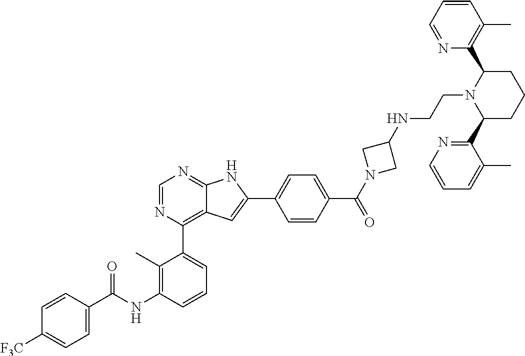 | LCMS (Agilent LCMS 1200-6120, Column: Waters X-Bridge C18 (50 mm*4.6 mm*3.5 μm)); Column Temperature: 40° C.; Flow Rate: 2.3 mL/min; Mobile Phase: from 95% [water + 10 mM NH₄HCO₃] and 5% [CH₃CN] to 5% [water + 10 mM NH₄HCO₃] and 100% [CH₃CN] in 1.75 min, then under this condition for 0.8 min, finally changed to 95% [water + 10 mM NH₄HCO₃] and 5% [CH₃CN] in 0.1 min and under this condition for 0.1 min). Purity: 100.00 %. Rt = 1.710 min; MS | ¹H NMR (400 MHz, DMSO-d₆) δ: 12.85 (s, 1H), 10.30 (s, 1H), 8.35 (s, 2H), 8.21 (d, J = 8.0 Hz, 2H), 8.07 (d, J = 8.4 Hz, 2H), 7.94 (d, J = 8.4 Hz, 2H), 7.61-7.42 (m, 8H), 7.16 (q, J = 4.4 Hz, 2H), 6.95 (s, 1H), 4.24 (d, J = 10.0 Hz, 2H), 4.01-3.98 (m, 1H), 3.76 (s, 1H), 3.20 (d, J = 12.0 Hz, 1H), 2.72-2.68 (m, 1H), 2.39-2.33 (m, 6H), 2.22 (s, 6H), 2.00-1.93 (m, 2H), 1.69-1.62 (m, 1H), 1.51 (d, J = 17.2 |
| | | Calcd.: 863.4; MS | Hz, 2H), 1.45-1.34 |
| | | Found: 864.4 [M + H]⁺. | (m, 1H), 1.20-1.09 |
| | | | (m, 2H). |
| I-38 | 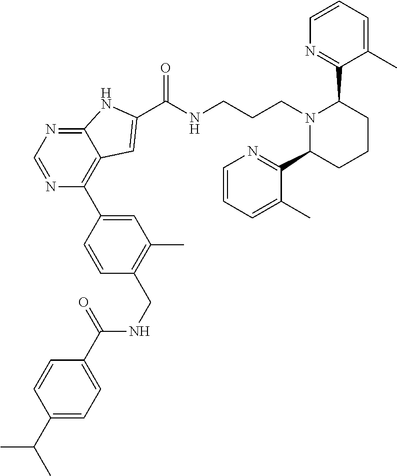 [SMILES: Cc1cc(-c2ncnc3[nH]c(C(=O)NCCCN4[C@@H](c5ncccc5C)CCC[C@H]4c4ncccc4C)cc23)ccc1CNC(=O)c1ccc(C(C)C)cc1] | LCMS (Agilent LCMS 1200-6120, Column: Waters X-Bridge C18 (50 mm*4.6 mm*3.5 μm); Column Temperature: 40° C.; Flow Rate: 2.0 mL/min; Mobile Phase: from 95% [water + 10 mM NH₄HCO₃] and 5% [CH₃CN] to 0% [water + 10 mM NH₄HCO₃] and 100% [CH₃CN] in 1.6 min, then under this condition for 1.4 min, finally changed to 95% [water + 10 mM NH₄HCO₃] and 5% [CH₃CN] in 0.1 min and under this condition for 0.7 min.) Purity: 100.00 %. Rt = 1.958 min; MS Calcd.: 734.4; MS Found: 735.5 [M + H]⁺. | ¹H NMR (400 MHz, DMSO-d₆) δ: 12.56 (s, 1H), 9.05 (t, J = 5.6 Hz, 1H), 8.87 (s, 1H), 8.21 (s, 3H), 7.97 (t, J = 8.8 Hz, 2H), 7.83 (s, 1H), 7.77 (d, J = 6.8 Hz, 1H), 7.47-7.39 (m, 6H), 6.95 (s, 2H), 4.57 (d, J = 5.2 Hz, 2H), 4.13 (t, J = 6.4 Hz, 2H), 2.99-2.95 (m, 1H), 2.67-2.50 (m, 6H), 2.50-2.49 (m, 5H), 2.33 (d, J = 1.6 Hz, 2H), 2.08 (m, 1H), 1.50-1.46 (m, 3H), 1.25 (d, J = 7.2 Hz, 6H), 1.21 (s, 2H). |
| I-39 | 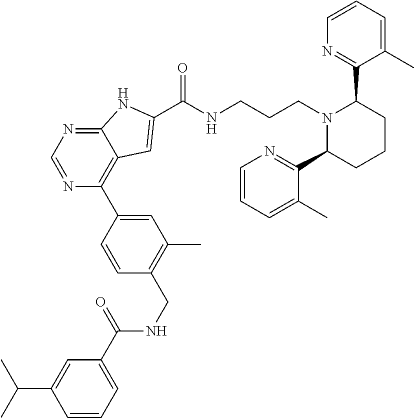 [SMILES: Cc1cc(-c2ncnc3[nH]c(C(=O)NCCCN4[C@@H](c5ncccc5C)CCC[C@H]4c4ncccc4C)cc23)ccc1CNC(=O)c1cccc(C(C)C)c1] | LCMS (Agilent LCMS 1200-6120, Column: Waters X-Bridge C18 (50 mm*4.6 mm*3.5 μm); Column Temperature: 40° C.; Flow Rate: 2.0 mL/min; Mobile Phase: from 95% [water + 10 mM NH₄HCO₃] and 5% [CH₃CN] to 0% [water + 10 mM NH₄HCO₃] and 100% [CH₃CN] in 1.6 min, then under this condition for 1.4 min, finally changed to 95% [water + 10 mM NH₄HCO₃] and 5% [CH₃CN] in 0.1 min and under this condition for 0.7 min.) Purity: 100.00 %. Rt = 1.958 min; MS Calcd.: 734.4; MS Found: 735.5 [M + H]⁺. | ¹H NMR (400 MHz, DMSO-d₆) δ: 12.56 (s, 1H), 9.05 (t, J = 5.6 Hz, 1H), 8.87 (s, 1H), 8.21 (s, 3H), 7.97 (t, J = 8.8 Hz, 2H), 7.83 (s, 1H), 7.77 (d, J = 6.8 Hz, 1H), 7.47-7.39 (m, 6H), 6.95 (s, 2H), 4.57 (d, J = 5.2 Hz, 2H), 4.13 (t, J = 6.4 Hz, 2H), 2.99-2.95 (m, 1H), 2.67-2.50 (m, 6H), 2.50-2.49 (m, 5H), 2.33 (d, J = 1.6 Hz, 2H), 2.08 (m, 1H), 1.50-1.46 (m, 3H), 1.25 (d, J = 7.2 Hz, 6H), 1.21 (s, 2H). |
| I-40 | 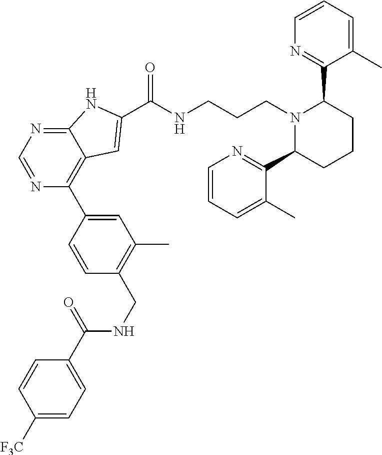 [SMILES: Cc1cc(-c2ncnc3[nH]c(C(=O)NCCCN4[C@@H](c5ncccc5C)CCC[C@H]4c4ncccc4C)cc23)ccc1CNC(=O)c1ccc(C(F)(F)F)cc1] | LCMS (Agilent LCMS 1200-6120, Column: Waters X-Bridge C18 (50 mm*4.6 mm*3.5 μm); Column Temperature: 40° C.; Flow Rate: 2.0 mL/min; Mobile Phase: from 95% [water + 10 mM NH₄HCO₃] and 5% [CH₃CN] to 0% [water + 10 mM NH₄HCO₃] and 100% [CH₃CN] in 1.6 min, then under this condition for 1.4 min, finally changed to 95% [water + 10 mM NH₄HCO₃] and 5% [CH₃CN] in 0.1 min and under this condition for 0.7 min.) Purity: 99.05 %. Rt = 1.92 min; MS Calcd.: 760.4; MS Found: 761.5 [M + H]⁺. | ¹H NMR (400 MHz, DMSO-d₆) δ: 12.56 (s, 1H), 9.05 (t, J = 5.6 Hz, 1H), 8.87 (s, 1H), 8.21 (s, 3H), 7.97 (t, J = 8.8 Hz, 2H), 7.83 (s, 1H), 7.77 (d, J = 6.8 Hz, 1H), 7.47-7.39 (m, 6H), 6.95 (s, 2H), 4.57 (d, J = 5.2 Hz, 2H), 4.13 (t, J = 6.4 Hz, 2H), 2.99-2.95 (m, 1H), 2.67-2.50 (m, 6H), 2.50-2.49 (m, 5H), 2.33 (d, J = 1.6 Hz, 2H), 2.08 (m, 1H), 1.50-1.46 (m, 3H), 1.21 (s, 2H). |
| I-41 | 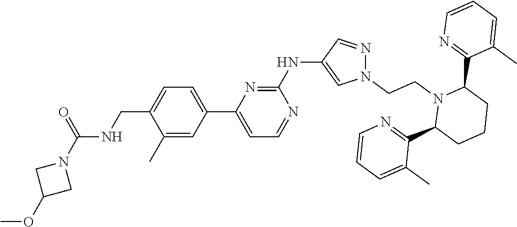 | LCMS (Agilent LCMS 1200-6120, Column: Waters X-Bridge C18 (50 mm*4.6 mm*3.5 μm); Column Temperature: 40° C.; Flow Rate: 2.0 mL/min; Mobile Phase: from 95% [water + 10 mM NH₄HCO₃] and 5% [CH₃CN] to 0% [water + 10 mM NH₄HCO₃] and 100% [CH₃CN] in 1.6 min, then under this | ¹H NMR (400 MHz, DMSO-d₆) δ: 9.33 (s, 1H), 8.47 (s, 1H), 8.36 (s, 2H), 7.90 (d, J = 7.6 Hz, 2H), 7.53 (s, 2H), 7.40 (s, 1H), 7.25-7.15 (m, 4H), 6.99 (s, 1H), 6.92 (s, 1H), 4.36 (d, J = 10.8 Hz, 2H), 4.25 (d, J = 5.2 Hz, 2H), 4.17-4.12 (m, 1H), |
| | | condition for 1.4 min, | 4.03-3.99 (m, 2H), |
| | | finally changed to 95% | 3.65 (dd, J = 4.0, |
| | | [water + 10 mM | 9.2 Hz, 2H), 3.19 |
| | | NH₄HCO₃] and 5% | (s, 3H), 2.74 (m, |
| | | [CH₃CN] in 0.1 min | 2H), 2.51 (s, 6H), |
| | | and under this | 2.5 (s, 2H), 2.38 |
| | | condition for 0.7 min.) | (s, 3H), 2.27-2.20 |
| | | Purity: 100.00%. Rt = | (m, 2H), 2.03-2.00 |
| | | 1.746 min; MS Calcd.: | (m, 1H), 1.73-1.70 |
| | | 686.4; MS Found: | (m, 1H), 1.49 (d, |
| | | 687.4 [M + H]⁺. | J = 12.0 Hz, 2H). |
| I-42 | 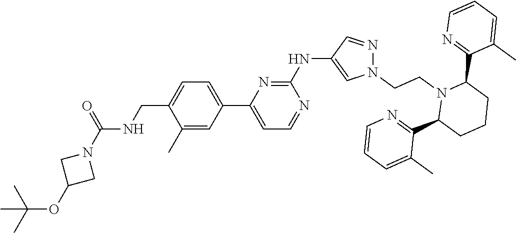 | LCMS (Agilent LCMS 1200-6120, Column: Waters X-Bridge C18 (50 mm*4.6 mm*3.5 μm); Column Temperature: 40° C.; Flow Rate: 2.0 mL/min; Mobile Phase: from 95% [water + 10 mM NH₄HCO₃] and 5% [CH₃CN] to 0% [water + 10 mM NH₄HCO₃] and 100% [CH₃CN] in 1.6 min, then under this condition for 1.4 min, | ¹H NMR (400 MHz, DMSO-d₆) δ: 9.33 (s, 1H), 8.46 (s, 1H), 8.36 (s, 2H), 7.90 (d, J = 7.6 Hz, 2H), 7.53 (s, 2H), 7.41 (s, 1H), 7.25-7.16 (m, 4H), 7.00 (s, 1H), 6.88 (s, 1H), 4.46-4.44 (m, 1H), 4.36 (d, J = 10.8 Hz, 2H), 4.25 (d, J = 4.0 Hz, 2H), 4.03 (t, J = 8.0 Hz, |
| | | finally changed to 95% | 2H), 3.62-3.59 (m, |
| | | [water + 10 mM | 2H), 2.73 (s, 2H), |
| | | NH₄HCO₃] and 5% | 2.68-2.51 (m, 6H), |
| | | [CH₃CN] in 0.1 min | 2.43 (s, 2H), 2.38 |
| | | and under this | (s, 3H), 2.23-2.20 |
| | | condition for 0.7 min.) | (m, 2H), 2.03-2.00 |
| | | Purity: 100.00%. Rt = | (m, 1H), 1.72 (d, |
| | | 1.885 min; MS Calcd.: | J = 12.4 Hz, 1H), |
| | | 728.4; MS Found: | 1.49 (d, J = 12.8 |
| | | 729.4 [M + H]⁺. | Hz, 2H), 1.11 (s, |
| | | | 9H). |
| I-43 | 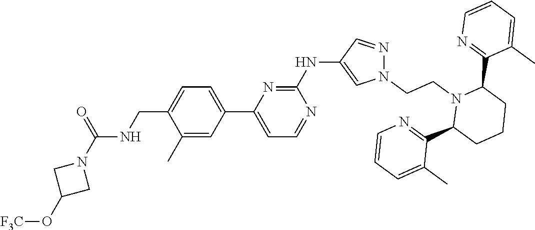 | LCMS (Agilent LCMS 1200-6120, Column: Waters X-Bridge C18 (50 mm*4.6 mm*3.5 μm); Column Temperature: 40° C.; Flow Rate: 2.0 mL/min; Mobile Phase: from 95% [water + 10 mM NH₄HCO₃] and 5% [CH₃CN] to 0% [water + 10 mM NH₄HCO₃] and 100% [CH₃CN] in 1.6 min, then under this | ¹H NMR (400 MHz, DMSO-d₆) δ: 9.33 (s, 1H), 8.47 (s, 1H), 8.36 (s, 2H), 7.90 (d, J = 7.6 Hz, 2H), 7.53 (s, 2H), 7.40 (s, 1H), 7.25-7.11 (m, 5H), 6.99 (s, 1H), 5.16 (s, 1H), 4.36 (d, J = 10.8 Hz, 2H), 4.28- 4.22 (m, 4H), 3.90 (dd, J = 3.6, 5.6 |
| | | condition for 1.4 min, | Hz, 2H), 2.73 (s, |
| | | finally changed to 95% | 2H), 2.51 (s, 6H), |
| | | [water + 10 mM | 2.50 (s, 2H), 2.38 |
| | | NH₄HCO₃] and 5% | (s, 3H), 2.22-2.20 |
| | | [CH₃CN] in 0.1 min | (m, 2H), 2.03-2.00 |
| | | and under this | (m, 1H), 1.73-1.70 |
| | | condition for 0.7 min.) | (m, 1H), 1.49 (d, |
| | | Purity: 100.00%. Rt = | J = 12.0 Hz, 2H). |
| | | 1.894 min; MS Calcd.: | |
| | | 740.4; MS Found: | |
| | | 741.4 [M + H]⁺. | |
| I-44 | 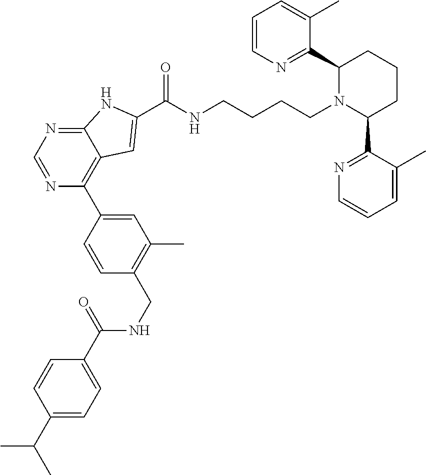 [SMILES: Cc1cc(-c2ncnc3[nH]c(C(=O)NCCCCN4[C@@H](c5ncccc5C)CCC[C@H]4c4ncccc4C)cc23)ccc1CNC(=O)c1ccc(C(C)C)cc1] | LCMS (Agilent LCMS 1200-6120, Column: Waters X-Bridge C18 (50 mm*4.6 mm*3.5 μm); Column Temperature: 40° C.; Flow Rate: 2.0 mL/min; Mobile Phase: from 95% [water + 10 mM NH₄HCO₃] and 5% [CH₃CN] to 0% [water + 10 mM NH₄HCO₃] and 100% [CH₃CN] in 1.6 min, then under this condition for 1.4 min, finally changed to 95% [water + 10 mM NH₄HCO₃] and 5% [CH₃CN] in 0.1 min and under this condition for 0.7 min.) Purity: 99.33 %. Rt = 1.990 min; MS Calcd.: 748.4; MS Found: 749.4 [M + H]⁺. | ¹H NMR (400 MHz, DMSO-d₆) δ: 12.56 (s, 1H), 9.05 (t, J = 5.6 Hz, 1H), 8.87 (s, 1H), 8.30 (s, 3H), 7.98 (t, J = 8.0 Hz, 2H), 7.83 (s, 1H), 7.77 (d, J = 6.8 Hz, 1H), 7.48-7.37 (m, 6H), 7.03 (s, 2H), 4.57 (d, J = 5.6 Hz, 2H), 4.18 (s, 2H), 2.97- 2.95 (m, 1H), 2.67- 2.50 (m, 6H), 2.50- 2.49 (m, 5H), 2.33 (s, 2H), 2.25-2.20 (m, 2H), 2.14-1.93 (m, 3H), 1.48-1.21 (m, 3H), 1.45 (d, J = 7.2 Hz, 6H), 1.21 (s, 2H). |
| I-45 | 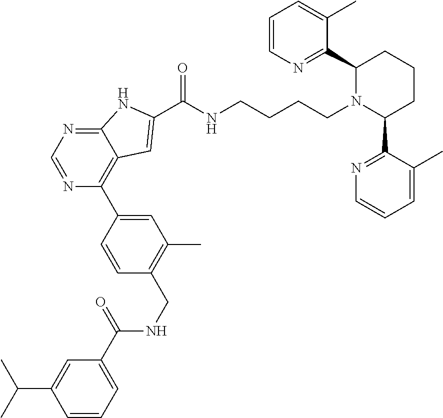 [SMILES: Cc1cc(-c2ncnc3[nH]c(C(=O)NCCCCN4[C@@H](c5ncccc5C)CCC[C@H]4c4ncccc4C)cc23)ccc1CNC(=O)c1cccc(C(C)C)c1] | LCMS (Agilent LCMS 1200-6120, Column: Waters X-Bridge C18 (50 mm*4.6 mm*3.5 μm); Column Temperature: 40° C.; Flow Rate: 2.0 mL/min; Mobile Phase: from 95% [water + 10 mM NH₄HCO₃] and 5% [CH₃CN] to 0% [water + 10 mM NH₄HCO₃] and 100% [CH₃CN] in 1.6 min, then under this condition for 1.4 min, finally changed to 95% [water + 10 mM NH₄HCO₃] and 5% [CH₃CN] in 0.1 min and under this condition for 0.7 min.) Purity: 99.33 %. Rt = 1.990 min; MS Calcd.: 748.4; MS Found: 749.4 [M + H]⁺. | ¹H NMR (400 MHz, DMSO-d₆) δ: 12.56 (s, 1H), 9.05 (t, J = 5.6 Hz, 1H), 8.87 (s, 1H), 8.30 (s, 3H), 7.98 (t, J = 8.0 Hz, 2H), 7.83 (s, 1H), 7.77 (d, J = 6.8 Hz, 1H), 7.48-7.37 (m, 6H), 7.03 (s, 2H), 4.57 (d, J = 5.6 Hz, 2H), 4.18 (s, 2H), 2.97- 2.95 (m, 1H), 2.67- 2.50 (m, 6H), 2.50- 2.49 (m, 5H), 2.33 (s, 2H), 2.25-2.20 (m, 2H), 2.14-1.93 (m, 3H), 1.48-1.21 (m, 3H), 1.45 (d, J = 7.2 Hz, 6H), 1.21 (s, 2H). |
| I-46 | 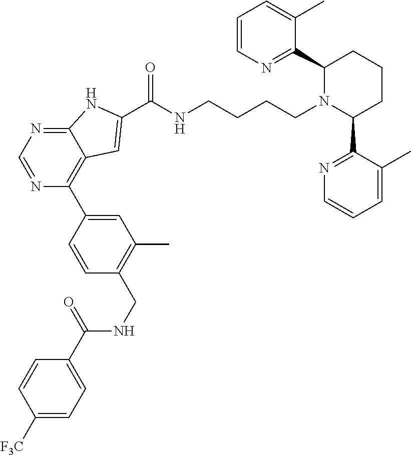 [SMILES: Cc1cc(-c2ncnc3[nH]c(C(=O)NCCCCN4[C@@H](c5ncccc5C)CCC[C@H]4c4ncccc4C)cc23)ccc1CNC(=O)c1ccc(C(F)(F)F)cc1] | LCMS (Agilent LCMS 1200-6120, Column: Waters X-Bridge C18 (50 mm*4.6 mm*3.5 μm); Column Temperature: 40° C.; Flow Rate: 2.0 mL/min; Mobile Phase: from 95% [water + 10 mM NH₄HCO₃] and 5% [CH₃CN] to 0% [water + 10 mM NH₄HCO₃] and 100% [CH₃CN] in 1.6 min, then under this condition for 1.4 min, finally changed to 95% [water + 10 mM NH₄HCO₃] and 5% [CH₃CN] in 0.1 min and under this condition for 0.7 min.) Purity: 100.00 %. Rt = 1.95 min; MS Calcd.: 774.4; MS Found: 775.4 [M + H]⁺. | ¹H NMR (400 MHz, DMSO-d₆) δ: 12.56 (s, 1H), 9.05 (t, J = 5.6 Hz, 1H), 8.87 (s, 1H), 8.30 (s, 3H), 7.98 (t, J = 8.0 Hz, 2H), 7.83 (s, 1H), 7.77 (d, J = 6.8 Hz, 1H), 7.48-7.37 (m, 6H), 7.03 (s, 2H), 4.57 (d, J = 5.6 Hz, 2H), 4.18 (s, 2H), 2.97- 2.95 (m, 1H), 2.67- 2.50 (m, 6H), 2.50- 2.49 (m, 5H), 2.33 (s, 2H), 2.25-2.20 (m, 2H), 2.14-1.93 (m, 3H), 1.48-1.21 (m, 3H), 1.21 (s, 2H). |
| I-47 | 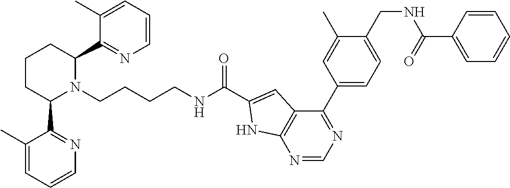 | LCMS (Agilent LCMS 1200-6120, Column: Waters X-Bridge C18 (50 mm*4.6 mm*3.5 μm); Column Temperature: 40° C.; Flow Rate: 2.0 mL/min; Mobile Phasc: from 95% [water + 10 mM NH₄HCO₃] and 5% [CH₃CN] to 0% [water + 10 mM NH₄HCO₃] | ¹H NMR (400 MHz, DMSO-d₆) δ: 12.61 (s, 1H), 9.07 (t, J = 5.6 Hz, 1H), 8.88 (s, 1H), 8.29 (s, 3H), 8.00- 7.95 (m, 4H), 7.59-7.40 (m, 7H), 7.03 (s, 2H), 4.58 (d, J = 5.6 Hz, 2H), 4.19 (s, 2H), 2.67 (s, 2H), 2.51 |
| | | and 100% [CH₃CN] in | (s, 6H), 2.50 (s, |
| | | 1.6 min, then under this | 3H), 2.26 (s, 2H), |
| | | condition for 1.4 min, | 2.12-2.08 (m, 2H), |
| | | finally changed to 95% | 1.94 (s, 1H), 1.93 |
| | | [water + 10 mM | (s, 1H), 1.49 (s, |
| | | NH₄HCO₃] and 5% | 2H), 0.67 (s, 2H), |
| | | [CH₃CN] in 0.1 min | 0.29-0.20 (m, 2H). |
| | | and under this | |
| | | condition for 0.7 min.) | |
| | | Purity: 100.00%. Rt = | |
| | | 2.011 min; MS Calcd.: | |
| | | 706.4; MS Found: | |
| | | 707.3 [M + H]⁺. | |
| I-48 | 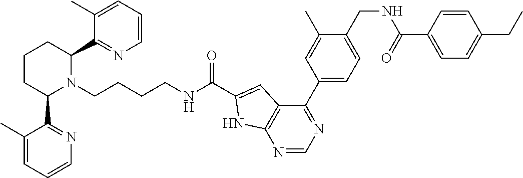 | LCMS (Agilent LCMS 1200-6120, Column: Waters X-Bridge C18 (50 mm*4.6 mm*3.5 μm); Column Temperature: 40° C.; Flow Rate: 2.0 mL/min; Mobile Phase: from 95% [water + 10 mM NH₄HCO₃] and 5% [CH₃CN] to 0% [water + | ¹H NMR (400 MHz, DMSO-d₆) δ: 12.61 (s, 1H), 8.99 (t, J = 5.6 Hz, 1H), 8.87 (s, 1H), 8.29 (s, 3H), 8.00- 7.95 (m, 2H), 7.88 (d, J = 8.0 Hz, 2H), 7.47-7.23 (m, 6H), 7.03 (s, 2H), 4.57 (d, J = 5.6 |
| | | 10 mM NH₄HCO₃] | Hz, 2H), 4.18 (s, |
| | | and 100% [CH₃CN] in | 2H), 2.70-2.60 (m, |
| | | 1.6 min, then under this | 3H), 2.50 (s, 6H), |
| | | condition for 1.4 min, | 2.49 (s, 3H), 2.33- |
| | | finally changed to 95% | 2.21 (m, 2H), |
| | | [water + 10 mM | 2.13-2.08 (m, 2H), |
| | | NH₄HCO₃] and 5% | 1.94 (s, 1H), 1.48 |
| | | [CH₃CN] in 0.1 min | (s, 1H), 1.49 (s, |
| | | and under this | 2H), 1.23-1.19 (m, |
| | | condition for 0.7 min.) | 3H), 1.17 (s, 1H), |
| | | Purity: 100.00%. Rt = | 0.67 (s, 2H), 0.29- |
| | | 1.938 min; MS Calcd.: | 0.20 (m, 2H). |
| | | 734.4; MS Found: | |
| | | 735.3 [M + H]⁺. | |
| I-49 | 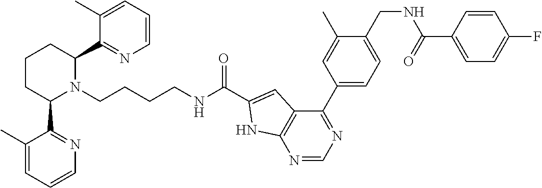 | LCMS (Agilent LCMS 1200-6120, Column: Waters X-Bridge C18 (50 mm*4.6 mm*3.5 μm); Column Temperature: 40° C.; Flow Rate: 2.0 mL/min; Mobile Phase: from 95% [water + 10 mM NH₄HCO₃] and 5% [CH₃CN] to 0% [water + 10 mM NH₄HCO₃] | ¹H NMR (400 MHz, DMSO-d₆) δ: 12.51 (s, 1H), 9.10 (t, J = 5.6 Hz, 1H), 8.87 (s, 1H), 8.30 (s, 3H), 8.04- 8.00 (m, 3H), 7.97 (d, J = 8.0 Hz, 1H), 7.52-7.45 (m, 2H), 7.40-7.33 (m, 4H), 7.03 (s, 2H), 4.57 (d, J = 5.2 |
| | | and 100% [CH₃CN] in | Hz, 2H), 4.19 (s, |
| | | 1.6 min, then under this | 2H), 2.68-2.67 (m, |
| | | condition for 1.4 min, | 2H), 2.49 (s, 9H), |
| | | finally changed to 95% | 2.34-2.26 (m, 2H), |
| | | [water + 10 mM | 2.11-2.02 (m, 2H), |
| | | NH₄HCO₃] and 5% | 1.94 (s, 1H), 1.64- |
| | | [CH₃CN] in 0.1 min | 1.57 (m, 1H), 1.47 |
| | | and under this | (s, 2H), 0.66 (s, |
| | | condition for 0.7 min.) | 2H), 0.29-0.20 (m, |
| | | Purity: 98.42%. Rt = | 2H). |
| | | 2.045 min; MS Calcd.: | |
| | | 724.4; MS Found: | |
| | | 725.3 [M + H]⁺. | |
| I-50 | 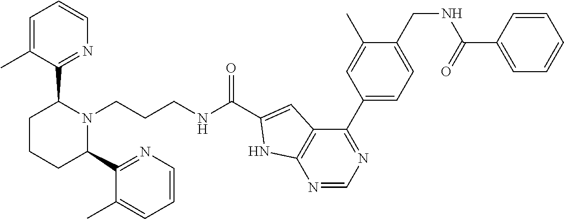 | LCMS (Agilent LCMS 1200-6120, Column: Waters X-Bridge C18 (50 mm*4.6 mm*3.5 μm); Column Temperature: 40° C.; Flow Rate: 2.0 mL/min; Mobile Phase: from 95% [water + 10 mM NH₄HCO₃] and 5% [CH₃CN] to 0% [water + 10 mM NH₄HCO₃] and 100% [CH₃CN] in 1.6 min, then under this | ¹H NMR (400 MHz, DMSO-d₆) δ: 12.55 (s, 1H), 9.08 (t, J = 5.6 Hz, 1H), 8.87 (s, 1H), 8.22-8.16 (m, 3H), 7.99-7.94 (m, 4H), 7.60-7.37 (m, 7H), 6.96 (s, 2H), 4.58 (d, J = 5.6 Hz, 2H), 4.13 (s, 2H), 2.51 (s, 6H), 2.49 (s, 5H), 2.40-2.33 (m, 2H), 2.10-2.07 |
| | | condition for 1.4 min, | (m, 2H), 1.92-1.89 |
| | | finally changed to 95% | (m, 1H), 1.62-1.58 |
| | | [water + 10 mM | (m, 1H), 1.51-1.49 |
| | | NH₄HCO₃] and 5% | (m, 2H), 0.62-0.59 |
| | | [CH₃CN] in 0.1 min | (m, 2H). |
| | | and under this | |
| | | condition for 0.7 min.) | |
| | | Purity: 100.00%. Rt = | |
| | | 1.784 min; MS Calcd.: | |
| | | 692.4; MS Found: | |
| | | 693.3 [M + H]⁺. | |
| I-51 | 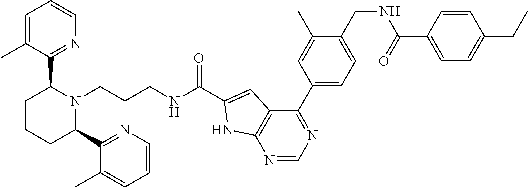 | LCMS (Agilent LCMS 1200-6120, Column: Waters X-Bridge C18 (50 mm*4.6 mm*3.5 μm); Column Temperature: 40° C.; Flow Rate: 2.0 mL/min; Mobile Phase: from 95% [water + 10 mM NH₄HCO₃] and 5% [CH₃CN] to 0% [water + 10 mM NH₄HCO₃] | ¹H NMR (400 MHz, DMSO-d₆) δ: 12.55 (s, 1H), 9.00 (t, J = 5.6 Hz, 1H), 8.87 (s, 1H), 8.21 (s, 3H), 7.98- 7.94 (m, 2H), 7.88 (d, J = 8.0 Hz, 2H), 7.46-7.33 (m, 6H), 6.95 (s, 2H), 4.56 (d, J = 5.6 Hz, 2H), 4.13 (s, |
| | | and 100% [CH₃CN] in | 2H), 2.71-2.621 |
| | | 1.6 min, then under this | (m, 2H), 2.51 (s, |
| | | condition for 1.4 min, | 9H), 2.48 (s, 2H), |
| | | finally changed to 95% | 2.34-2.33 (m, 2H), |
| | | [water + 10 mM | 1.89 (s, 2H), 1.61 |
| | | NH₄HCO₃] and 5% | (s, 1H), 1.61-1.56 |
| | | [CH₃CN] in 0.1 min | (m, 1H), 1.50 (s, |
| | | and under this | 2H), 1.21 (t, J = |
| | | condition for 0.7 min.) | 7.6 Hz, 3H), 0.65- |
| | | Purity: 100.00%. Rt = | 0.53 (m, 2H). |
| | | 1.906 min; MS Calcd.: | |
| | | 720.4; MS Found: | |
| | | 721.3 [M + H]⁺. | |
| I-52 | 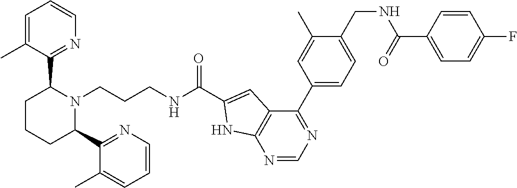 | LCMS (Agilent LCMS 1200-6120, Column: Waters X-Bridge C18 (50 mm*4.6 mm*3.5 μm); Column Temperature: 40° C.; Flow Rate: 2.0 mL/min; Mobile Phase: from 95% [water + 10 mM NH₄HCO₃] and 5% [CH₃CN] to 0% [water + 10 mM NH₄HCO₃] | ¹H NMR (400 MHz, DMSO-d₆): 12.56 (s, 1H), 9.10 (t, J = 6.0 Hz, 1H), 8.87 (s, 1H), 8.21 (s, 3H), 8.04- 8.00 (m, 3H), 7.96 (d, J = 8.0 Hz, 1H), 7.46 (d, J = 8.0 Hz, 1H), 7.41- 7.33 (m, 5H), 6.96 (s, 2H), 4.57 (d, J = |
| | | and 100% [CH₃CN] in | 5.6 Hz, 2H), |
| | | 1.6 min, then under this | 4.14 (s, 2H), 2.51 |
| | | condition for 1.4 min, | (s, 2H), 2.49 (s, |
| | | finally changed to 95% | 9H), 2.34-2.32 (m, |
| | | [water + 10 mM | 2H), 2.20-2.07 (m, |
| | | NH₄HCO₃] and 5% | 2H), 1.98-1.89 (m, |
| | | [CH₃CN] in 0.1 min | 1H), 1.59 (s, 1H), |
| | | and under this | 1.50-1.48 (m, 2H), |
| | | condition for 0.7 min.) | 0.59 (s, 2H). |
| | | Purity: 100.00%. Rt = | |
| | | 2.000 min; MS Calcd.: | |
| | | 710.4; MS Found: | |
| | | 711.4 [M + H]⁺. | |
| I-53 | 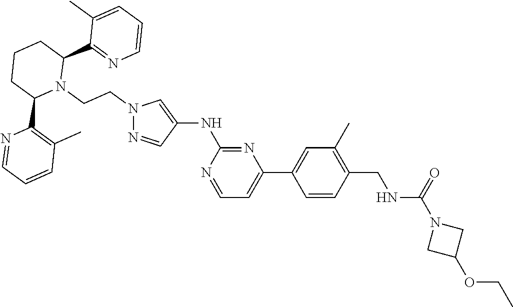 | LCMS (Agilent LCMS 1200-6120, Column: Waters X-Bridge C18 (50 mm*4.6 mm*3.5 μm); Column Temperature: 40° C.; Flow Rate: 2.0 mL/min; Mobile Phase: from 95% [water + 10 mM NH₄HCO₃] and 5% [CH₃CN] to 0% [water + 10 mM NH₄HCO₃] and 100% [CH₃CN] in 1.6 min, then under this condition for 1.4 min, finally changed to 95% [water + 10 mM NH₄HCO₃] and 5% [CH₃CN] in 0.1 min and | ¹H NMR (400 MHz, DMSO-d₆) δ: 9.32 (s, 1H), 8.46 (s, 1H), 8.36 (s, 2H), 7.91 (d, J = 8.0 Hz, 2H), 7.53 (s, 2H), 7.40 (s, 1H), 7.24-7.16 (m, 4H), 6.90 (s, 1H), 6.70 (s, 1H), 4.35 (d, J = 10.8 Hz, 2H), 4.26- 4.20 (m, 3H), 4.04-4.01 (m, 2H), 3.65 (dd, J = 9.2, 4.4 Hz, 2H), 3.41- 3.66 (m, 2H), 2.73 (br, 2H), 2.51 (s, 6H), 2.45-2.42 (m, |
| | | under this condition for | 2H), 2.38 (s, 3H), |
| | | 0.7 min.) Purity: | 2.26-2.20 (m, 2H), |
| | | 100.00%. Rt = 1.79 | 2.01-2.00 (m, 1H), |
| | | min; MS Calcd.: 700.4; | 1.72-1.69 (m, 1H), |
| | | MS Found: 701.3 [M + | 1.51-1.48 (m, 2H), |
| | | H]⁺. | 1.12 (t, J = 4.8 Hz, |
| | | | 3H). |
| I-54 | 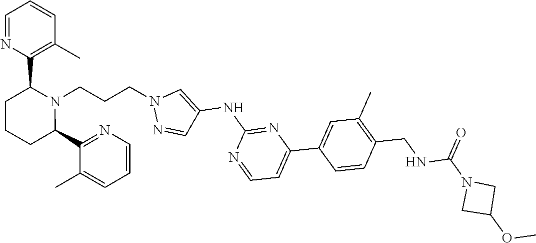 | LCMS (Agilent LCMS 1200-6120, Column: Waters X-Bridge C18 (50 mm*4.6 mm*3.5 μm); Column Temperature: 40° C.; Flow Rate: 2.0 mL/min; Mobile Phase: from 95% [water + 10 mM NH₄HCO₃] and 5% [CH₃CN] to 0% [water + 10 mM NH₄HCO₃] and 100% [CH₃CN] in 1.6 min, then under this condition for 1.4 min, | ¹H NMR (400 MHz, DMSO-d₆) δ: 9.32 (s, 1H), 8.45 (s, 1H), 8.36 (s, 2H), 7.91 (d, J = 8.0 Hz, 2H), 7.52 (s, 2H), 7.40 (s, 1H), 7.24-7.16 (m, 4H), 6.90 (s, 1H), 6.71 (s, 1H), 4.34-4.30 (m, 3H), 4.27-4.20 (m, 2H), 4.05-4.01 (m, 2H), 3.65 (dd, J = 9.2, 4.4 Hz, 2H), 3.19 |
| | | finally changed to 95% | (s, 3H), 2.51 (s, |
| | | [water + 10 mM | 6H), 2.45-2.38 (m, |
| | | NH₄HCO₃] and 5% | 5H), 2.26-2.20 (m, |
| | | [CH₃CN] in 0.1 min | 2H), 2.01-2.00 (m, |
| | | and under this | 1H), 1.72-1.69 (m, |
| | | condition for 0.7 min.) | 1H), 1.51-1.48 (m, |
| | | Purity: 100.00%. Rt = | 2H), 0.63 (br, 2H). |
| | | 1.890 min; MS Calcd.: | |
| | | 700.4; MS Found: | |
| | | 701.4 [M + H]⁺. | |
| I-55 | 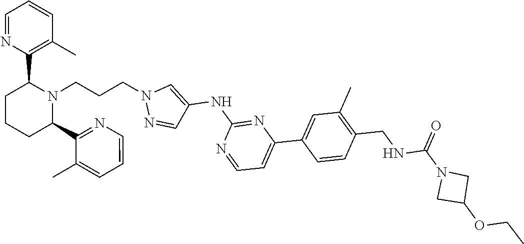 | LCMS (Agilent LCMS 1200-6120, Column: Waters X-Bridge C18 (50 mm*4.6 mm*3.5 μm); Column Temperature: 40° C.; Flow Rate: 2.0 mL/min; Mobile Phase: from 95% [water + 10 mM NH₄HCO₃] and 5% [CH₃CN] to 0% [water + 10 mM NH₄HCO₃] and 100% [CH₃CN] in 1.6 min, then under this condition for 1.4 min, | ¹H NMR (400 MHz, DMSO-d₆) δ: 9.36 (s, 1H), 8.46 (s, 1H), 8.30 (s, 2H), 7.90 (d, J = 8.0 Hz, 2H), 7.53-7.43 (s, 4H), 7.40 (s, 1H), 7.24- 7.16 (m, 1H), 6.90 (br, 2H), 6.70 (s, 1H), 4.35-4.30 (m, 4H), 4.26-4.20 (m, 2H), 4.04-4.01 (m, 2H), 3.65 (dd, J = 9.2, 4.4 Hz, 2H), |
| | | finally changed to 95% | 3.12 (br, 2H), 2.51 |
| | | [water + 10 mM | (s, 6H), 2.45-2.38 |
| | | NH₄HCO₃] and 5% | (m, 5H), 2.26-2.20 |
| | | [CH₃CN] in 0.1 min | (m, 2H), 2.01-2.00 |
| | | and under this | (m, 1H), 1.72-1.69 |
| | | condition for 0.7 min.) | (m, 1H), 1.51-1.48 |
| | | Purity: 100.00%. Rt = | (m, 2H), 1.12 (t, |
| | | 1.76 min; MS Calcd.: | J = 4.8 Hz, 3H), |
| | | 714.4; MS Found: | 0.72 (br, 2H). |
| | | 715.3 [M + H]⁺. | |
| I-56 | 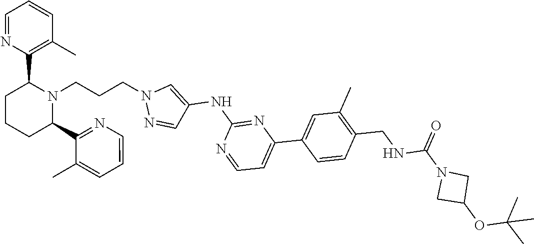 | LCMS (Agilent LCMS 1200-6120, Column: Waters X-Bridge C18 (50 mm*4.6 mm*3.5 μm); Column Temperature: 40° C.; Flow Rate: 2.0 mL/min; Mobile Phase: from 95% [water + 10 mM NH₄HCO₃] and 5% [CH₃CN] to 0% [water + 10 mM NH₄HCO₃] and 100% [CH₃CN] in 1.6 min, then under this condition for 1.4 min, | ¹H NMR (400 MHz, DMSO-d₆) δ: 9.36 (s, 1H), 8.45 (d, J = 4.4 Hz, 1H), 8.33 (s, 2H), 7.93 (d, J = 7.2 Hz, 2H), 7.46 (s, 2H), 7.49 (d, J = 6.8 Hz, 2H), 7.31 (s, 1H), 7.26 (d, J = 5.2 Hz, 1H), 7.08 (t, J = 5.2 Hz, 3H), 5.20- 5.14 (m, 1H), 2.23 (q, J = 6.8 Hz, |
| | | finally changed to 95% | 6H), 3.91 (dd, J = |
| | | [water + 10 mM | 3.6, 9.6 Hz, 2H), |
| | | NH₄HCO₃] and 5% | 3.24-3.09 (m, 2H), |
| | | [CH₃CN] in 0.1 min | 2.52 (d, J = 2.0 |
| | | and under this | Hz, 6H), 2.41- |
| | | condition for 0.7 min.) | 2.31 (m, 5H), |
| | | Purity: 99.58%. Rt = | 2.28-2.16 (s, 2H), |
| | | 1.87 min; MS Calcd.: | 2.16-2.06 (m, 1H), |
| | | 742.4; MS Found: | 1.69-1.62 (m, 1H), |
| | | 743.2 [M + H]⁺. | 1.52-1.46 (m, 2H), |
| | | | 1.11 (s, 9H), 0.70- |
| | | | 0.64 (m, 2H). |
| I-57 | 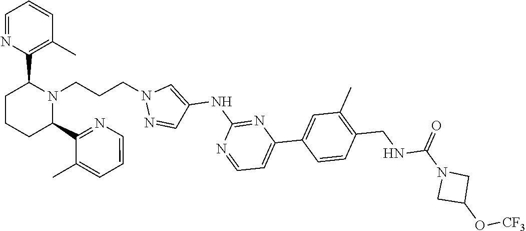 | LCMS (Agilent LCMS 1200-6120, Column: Waters X-Bridge C18 (50 mm*4.6 mm*3.5 μm); Column Temperature: 40° C.; Flow Rate: 2.0 mL/min; Mobile Phase: from 95% [water + 10 mM NH₄HCO₃] and 5% [CH₃CN] to 0% [water + 10 mM NH₄HCO₃] and 100% [CH₃CN] in 1.6 min, then under this | ¹H NMR (400 MHz, DMSO-d₆): 9.36 (s, 1H), 8.46 (d, J = 4.4 Hz, 1H), 8.34 (s, 2H), 7.91 (d, J = 7.2 Hz, 2H), 7.46 (s, 2H), 7.48 (d, J = 6.8 Hz, 2H), 7.31 (s, 1H), 7.25 (d, J = 5.2 Hz, 1H), 7.08 (t, J = 5.2 Hz, 3H), 5.20- 5.14 (m, 1H), 2.24 |
| | | condition for 1.4 min, | (q, J = 6.8 Hz, |
| | | finally changed to 95% | 6H), 3.91 (dd, J = |
| | | [water + 10 mM | 3.6, 9.6 Hz, 2H), |
| | | NH₄HCO₃] and 5% | 3.24-3.09 (m, 2H), |
| | | [CH₃CN] in 0.1 min and | 2.52 (d, J = 2.0 |
| | | under this condition for | Hz, 6H), 2.41- |
| | | 0.7 min.) Purity: | 2.31 (m, 5H), |
| | | 100.00%. Rt = 1.874 | 2.28-2.16 (s, 2H), |
| | | min; MS Calcd.: 754.4; | 2.14-2.06 (m, 1H), |
| | | MS Found: 755.3 [M + | 1.68-1.62 (m, 1H), |
| | | H]. | 1.50-1.46 (m, 2H), |
| | | | 0.70-0.64 (m, 2H). |
| I-58 | 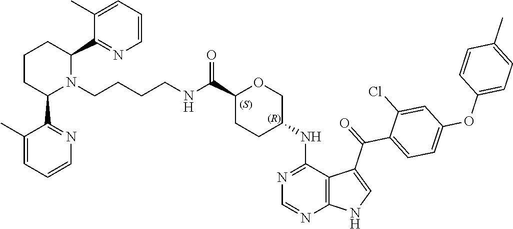 | LCMS (Agilent LCMS 1200-6120, Column: Waters X-Bridge C18 (50 mm*4.6 mm*3.5 μm); Column Temperature: 40° C.; Flow Rate: 2.0 mL/min; Mobile Phase: from 95% [water + 10 mM NH₄HCO₃] and 5% [CH₃CN] to 0% [water + 10 mM NH₄HCO₃] and 100% [CH₃CN] in 1.6 min, then under this | ¹H NMR (400 MHz, DMSO-d₆): 12.76 (s, 1H), 8.62 (d, J = 7.2 Hz, 1H), 8.35 (s, 2H), 8.26 (s, 1H), 7.63 (s, 1H), 7.55 (t, J = 4.4 Hz, 3H), 7.28 (d, J = 8.4 Hz, 2H), 7.19 (t, J = 11.2 Hz, 3H), 7.13 (d, J = 2.0 Hz, 1H), 7.12 (d J = 8.4 Hz, 1H), |
| | | condition for 1.4 min, | 7.11 (d, J = 8.4 |
| | | finally changed to 95% | Hz, 2H), 6.99- |
| | | [water + 10 mM | 6.96 (m, 1H), 4.21 |
| | | NH₄HCO₃] and 5% | (d, J = 9.6 Hz, |
| | | [CH₃CN] in 0.1 min and | 4H), 3.71 (d, J = |
| | | under this condition for | 10.4 Hz, 1H), 3.22 |
| | | 0.7 min.) Purity: | (t, J = 10.4 Hz, |
| | | 99.34%. Rt = 2.088 | 1H), 2.67 (s, 6H), |
| | | min; MS Calcd.: 826.4; | 2.54 (s, 2H), 2.33 |
| | | MS Found: 827.2 [M + | (s, 3H), 2.19-2.08 |
| | | H]. | (m, 5H), 2.02-1.93 |
| | | | (m, 2H), 1.66-1.60 |
| | | | (m, 2H), 1.52-1.41 |
| | | | (m, 2H), 1.34 (s, |
| | | | 2H), 0.48 (s, 2H). |
| I-59 | 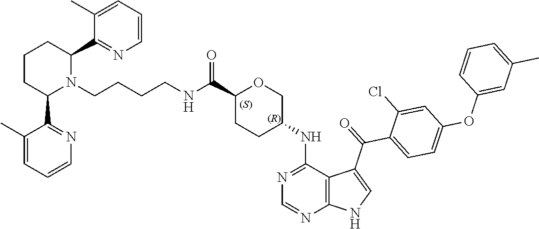 | LCMS (Agilent LCMS 1200-6120, Column: Waters X-Bridge C18 (50 mm*4.6 mm*3.5 μm); Column Temperature: 40° C.; Flow Rate: 2.0 mL/min; Mobile Phasc: from 95% [water + 10 mM NH₄HCO₃] and 5% [CH₃CN] to 0% [water + 10 mM NH₄HCO₃] and 100% [CH₃CN] in 1.6 min, then under this | ¹H NMR (400 MHz, DMSO-d₆) δ: 12.75 (s, 1H), 8.62 (d, J = 7.2 Hz, 1H), 8.35 (d, J = 3.2 Hz, 2H), 8.26 (s, 1H), 7.64 (s, 1H), 7.54 (q, J = 4.0 Hz, 3H), 7.39 (d, J = 7.2 Hz, 1H), 7.33- 7.29 (m, 1H), 7.23-7.16 (m, 4H), 7.09 (dd, J = 8.0, |
| | | condition for 1.4 min, | 2.4 Hz, 2H), 6.88 |
| | | finally changed to 95% | (dd, J = 2.4, 2.4 |
| | | [water + 10 mM | Hz, 2H), 4.23- |
| | | NH₄HCO₃] and 5% | 4.12 (m, 4H), 3.71 |
| | | [CH₃CN] in 0.1 min and | (d, J = 9.6 Hz, |
| | | under this condition for | 1H), 3.22 (t, J = |
| | | 0.7 min.) Purity: | 10.0 Hz, 1H), 2.55 |
| | | 99.78%. Rt = 2.153 | (s, 6H), 2.51 (s, |
| | | min; MS Calcd.: 826.4; | 1H), 2.20-2.08 (m, |
| | | MS Found: 827.2 [M + | 8H), 2.02-1.95 (m, |
| | | H]. | 2H), 1.69-1.60 (m, |
| | | | 2H), 1.51 (s, 2H), |
| | | | 1.41-1.35 (m, 1H), |
| | | | 0.55 (m, 2H), |
| | | | 0.27-0.20 (m, 2H). |
| I-60 | 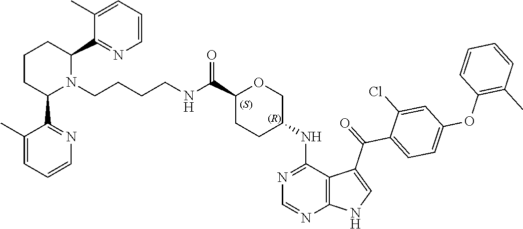 | LCMS (Agilent LCMS 1200-6120, Column: Waters X-Bridge C18 (50 mm*4.6 mm*3.5 μm); Column Temperature: 40° C.; Flow Rate: 2.0 mL/min; Mobile Phase: from 95% [water + 10 mM NH₄HCO₃] and 5% [CH₃CN] to 0% [water + 10 mM NH₄HCO₃] and 100% [CH₃CN] in 1.6 min, then under this | ¹H NMR (400 MHz, DMSO-d₆) δ: 12.77 (s, 1H), 8.62 (s, 1H), 8.35 (s, 2H), 8.27 (s, 1H), 7.65 (s, 1H), 7.56 (t, J = 8.4 Hz, 3H), 7.35 (t, J = 8.0 Hz, 1H), 7.18 (t, J = 8.0 Hz, 4H), 7.13 (d, J = 2.0 Hz, 1H), 7.02- 6.95 (m, 3H), 4.23-4.13 (m, 4H), |
| | | condition for 1.4 min, | 3.71 (d, J = 11.2 |
| | | finally changed to 95% | Hz, 1H), 3.22 (t, |
| | | [water + 10 mM | J = 10.0 Hz, 1H), |
| | | NH₄HCO₃] and 5% | 2.55 (s, 6H), 2.49 |
| | | [CH₃CN] in 0.1 min and | (s, 2H), 2.34 (s, |
| | | under this condition for | 3H), 2.20-2.08 (m, |
| | | 0.7 min.) Purity: 100%. | 5H), 2.02-1.95 (m, |
| | | Rt = 2.162 min; MS | 2H), 1.69-1.57 (m, |
| | | Calcd.: 826.4; MS | 2H), 1.51 (s, 2H), |
| | | Found: 827.2 [M + H]. | 1.44-1.35 (m, 1H), |
| | | | 0.56 (s, 2H), 2.28- |
| | | | 0.21 (m, 2H). |
| I-61 | 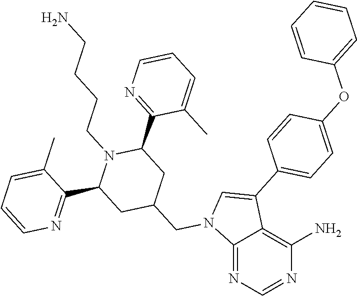 | LCMS (Agilent LCMS 1200-6120, Column: Waters X-Bridge C18 (50 mm*4.6 mm*3.5 μm); Column Temperature: 40° C.; Flow Rate: 2.0 mL/min; Mobile Phase: from 95% [water + 10 mM NH₄HCO₃] and 5% [CH₃CN] to 0% [water + 10 mM NH₄HCO₃] and 100% [CH₃CN] in 1.6 min, then under this condition for 1.4 min, finally changed to 95% [water + 10 mM NH₄HCO₃] and 5% | ¹H NMR (400 MHz, DMSO-d₆): δ 8.47 (d, J = 7.6 Hz, 1H), 8.25 (d, J = 3.6 Hz, 1H), 8.16 (s, 1H), 8.12 (s, 1H), 7.16 (t, J = 7.6 Hz, 2), 7.42 (q, J = 4.0 Hz, 4H), 7.37 (s 1H), 7.28- 7.21 (m, 2H), 7.16 (t, J = 7.6 Hz, 1H), 7.09 (t, J = 8.4 Hz, 4H), 6.31 (q, J = 4.4 Hz, 1H), 4.17 (s, 1H), 4.08 (s, 1H), 3.37-3.17 (m, 2H), 2.69-2.57 (m, |
| | | [CH₃CN] in 0.1 min and | 3H), 2.45 (t, J = |
| | | under this condition for | 7.2 Hz, 5H), 2.26 |
| | | 0.7 min.) Purity: 100.00 | (q, J = 12.4 Hz, |
| | | %. Rt = 1.012 min; MS | 5H), 2.12-1.88 (m, |
| | | Calcd.: 652.4; MS | 4H), 1.50-1.39 (m, |
| | | Found: 653.3 [M + H]⁺. | 4H). |
| I-64 | 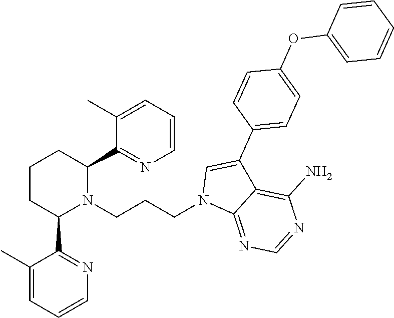 | LCMS (Agilent LCMS 1200-6120, Column: Waters X-Bridge C18 (50 mm*4.6 mm*3.5 μm); Column Temperature: 40° C.; Flow Rate: 2.0 mL/min; Mobile Phase: from 95% [water + 10 mM NH₄HCO₃] and 5% [CH₃CN] to 0% [water + 10 mM NH₄HCO₃] and 100% [CH₃CN] in 1.6 min, then under this condition for 1.4 min, finally changed to 95% [water + 10 mM NH₄HCO₃] and 5% [CH₃CN] in 0.1 min | ¹H NMR (400 MHz, DMSO-d₆) δ: 8.20 (s, 2H), 8.02 (s, 1H), 7.45- 7.34 (m, 6H), 7.18- 7.10 (m, 5H), 7.00- 6.97 (m, 2H), 6.65 (s, 1H), 6.02 (s, 2H), 4.14 (s, 2H), 3.46-3.41 (m, 2H), 2.20 (s, 6H), 2.08- 2.05 (d, J = 9.2 Hz, 2H), 2.00-1.88 (m, 3H), 1.63 (s, 1H), 1.47-1.45 (d, J = 10.4, 2H), 0.78 (s, 2H). |
| | | and under this | |
| | | condition for 0.7 min.) | |
| | | Purity: 100.00%. Rt = | |
| | | 2.015 min; MS Calcd.: | |
| | | 609.3; MS Found: | |
| | | 610.1 [M + H]⁺. | |
| I-66 | 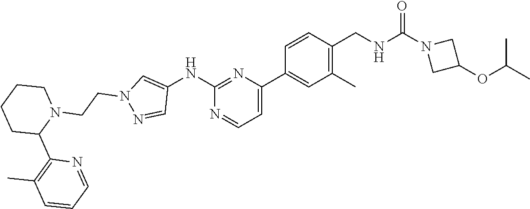 | LCMS (Agilent LCMS 1200-6120, Column: Waters X-Bridge C18 (50 mm*4.6 mm*3.5 μm); Column Temperature: 40° C.; Flow Rate: 2.0 mL/min; Mobile Phase: from 95% [water + 10 mM NH₄HCO₃] and 5% [CH₃CN] to 0% [water + 10 mM NH₄HCO₃] and 100% [CH₃CN] in | ¹H NMR (400 MHz, DMSO-d₆) δ: 9.44 (s, 1H), 8.44 (d, J = 5.2 Hz, 1H), 8.32 (d, J = 3.6 Hz, 1H), 7.92 (d, J = 8.8 Hz, 2H), 7.82 (s, 1H), 7.45 (s, 2H), 7.36 (d, J = 8.0 Hz, 1H), 7.24 (d, J = 5.2 Hz, 1H), 7.11 (s, 1H), 6.87 |
| | | 1.6 min, then under this | (t, J = 5.6 Hz, 1H), |
| | | condition for 1.4 min, | 4.34-4.29 (m, 1H), |
| | | finally changed to 95% | 4.23 (d, J = 5.6 |
| | | [water + 10 mM | Hz, 2H), 4.06- |
| | | NH₄HCO₃] and 5% | 3.98 (m, 4H), |
| | | [CH₃CN] in 0.1 min | 3.64-3.55 (m, 4H), |
| | | and under this | 3.15 (d, J = 11.2 |
| | | condition for 0.7 min.) | Hz, 1H), 2.68- |
| | | Purity: 97.51%. Rt = | 2.62 (m, 2H), 2.36 |
| | | 1.776 min; MS Calcd.: | (s, 3H), 2.32 (m, |
| | | 623.4; MS Found: | 3H), 2.22-1.17 (m, |
| | | 624.3 [M + H]⁺. | 1H), 1.79-1.74 (m, |
| | | | 2H), 1.59-1.53 (m, |
| | | | 3H), 1.40-1.34 (m, |
| | | | 1H), 1.08 (d, J = |
| | | | 6.0 Hz, 6H). |
| I-71 | 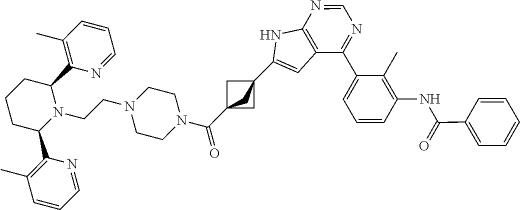 | LCMS (Agilent LCMS 1200-6120, Column: Waters X-Bridge C18 (50 mm*4.6 mm*3.5 μm); Column Temperature: 40° C.; Flow Rate: 2.0 mL/min; Mobile Phase: from 95% [water + 10 mM NH₄HCO₃] and 5% [CH₃CN] to 0% [water + 10 mM NH₄HCO₃] and 100% | ¹H NMR (400 MHz, DMSO-d₆) δ: 12.29 (br, 1H), 10.03 (s, 1H), 8.79 (s, 1H), 8.36 (d, J = 3.6 Hz, 2H), 8.02-8.00 (m, 2H), 7.63-7.48 (m, 6H), 7.40-7.32 (m, 2H), 7.20-7.17 (m, 2H), 6.03 (s, 1H), 4.28 (d, J = 10.0 Hz, 2H), 3.28 (br, 2H), |
| | | [CH₃CN] in 1.6 min, | 3.24 (br, 2H), 2.53 |
| | | then under this | (s, 6H), 2.51-2.48 |
| | | condition for 1.4 min, | (m, 2H), 2.30 (s, |
| | | finally changed to 95% | 6H), 2.24-2.18 (m, |
| | | [water + 10 mM | 2H), 2.12 (s, 3H), |
| | | NH₄HCO₃] and 5% | 2.09-2.01 (m, 1H), |
| | | [CH₃CN] in 0.1 min | 1.70-1.45 (m, 7H), |
| | | and under this | 0.89 (br, 2H). |
| | | condition for 0.7 min.) | |
| | | Purity: 100.00%. Rt = | |
| | | 2.00 min; MS Calcd.: | |
| | | 799.4; MS Found: | |
| | | 800.2 [M + H]⁺. | |
| I-73 | 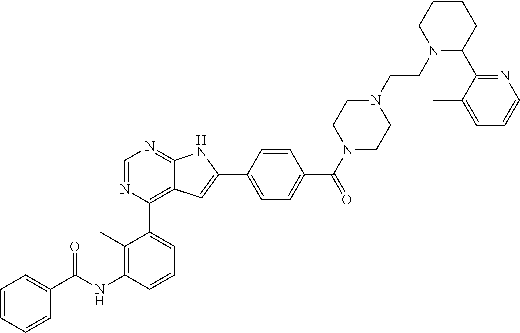 | LCMS (Agilent LCMS 1200-6120, Column: Waters X-Bridge C18 (50 mm*4.6 mm*3.5 μm); Column Temperature: 40° C.; Flow Rate: 2.0 mL/min; Mobile Phase: from 95% [water + 10 mM NH₄HCO₃] and 5% [CH₃CN] to 0% [water + 10 mM NH₄HCO₃] and 100% [CH₃CN] in 1.6 min, then under this condition for 1.4 min, finally changed to 95% [water + 10 mM NH₄HCO₃] and 5% [CH₃CN] in 0.1 min | ¹H NMR (400 MHz, DMSO-d₆) δ: 12.81 (s, 1H), 10.04 (s, 1H), 8.87 (s, 1H), 8.32 (t, J = 3.6 Hz, 1H), 8.03 (d, J = 7.2 Hz, 4H), 7.63-7.50 (m, 5H), 7.47-7.40 (m, 4H), 7.14 (dd, J = 4.8, 7.6 Hz, 1H), 6.88 (s, 1H), 3.50- 3.47 (m, 3H), 3.27-3.24 (m, 2H), 3.12 (d, J = 11.2 Hz, 1H), 2.43 (s, 3H), 2.37-2.27 (m, 4H), 2.21 (s, 6H), 2.13-2.05 (m, 2H), 1.78-1.73 (m, 2H), |
| | | and under this | 1.59-1.51 (m, 3H), |
| | | condition for 0.7 min.) | 1.35-1.31 (m, 1H). |
| | | Purity: 95.75%. Rt = | |
| | | 1.738 min; MS Calcd.: | |
| | | 718.4; MS Found: | |
| | | 720.3 [M + 2H]⁺. | |
| I-74 | 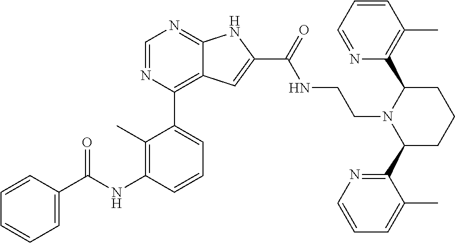 | LCMS (Agilent LCMS 1200-6120, Column: Waters X-Bridge C18 (50 mm*4.6 mm*3.5 μm); Column Temperature: 40° C.; Flow Rate: 2.0 mL/min; Mobile Phase: from 95% [water + 10 mM NH₄HCO₃] and 5% [CH₃CN] to 0% [water + 10 mM NH₄HCO₃] and 100% [CH₃CN] in 1.6 min, then under this condition for 1.4 min, | ¹H NMR (400 MHz, DMSO-d₆) δ: 12.45 (s, 1H), 10.04 (s, 1H), 8.86 (d, J = 5.2 Hz, 1H), 8.31 (d, J = 3.6 Hz, 2H), 8.03-8.00 (m, 2H), 7.75 (s, 1H), 7.63-7.59 (m, 4H), 7.57-7.33 (m, 4H), 7.10-7.07 (m, 2H), 6.81 (s, 1H), 4.26 (d, J = 8.4 Hz, 2H), 2.54 (s, 6H), 2.49 (s, 2H), 2.12 (s, |
| | | finally changed to 95% | 5H), 2.08-2.02 (m, |
| | | [water + 10 mM | 2H), 2.00-1.94 (m, |
| | | NH₄HCO₃] and 5% | 1H), 1.68-1.65 (m, |
| | | [CH₃CN] in 0.1 min and | 1H), 1.51 (d, J = |
| | | under this condition for | 12.0 Hz, 2H). |
| | | 0.7 min.) Purity: | |
| | | 99.50%. Rt = 1.880 | |
| | | min; MS Calcd.: 664.3; | |
| | | MS Found: 665.3 [M + | |
| | | H]⁺. | |
| I-75 | 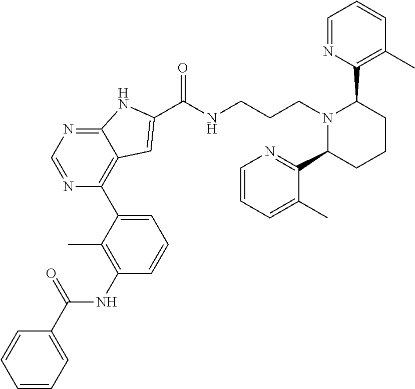 [SMILES: Cc1cccnc1[C@H]1CCC[C@@H](c2ncccc2C)N1CCCNC(=O)c1cc2c(-c3cccc(NC(=O)c4ccccc4)c3C)ncnc2[nH]1] | LCMS (Agilent LCMS 1200-6120, Column: Waters X-Bridge C18 (50 mm*4.6 mm*3.5 μm); Column Temperature: 40° C.; Flow Rate: 2.0 mL/min; Mobile Phase: from 95% [water + 10 mM NH₄HCO₃] and 5% [CH₃CN] to 0% [water + 10 mM NH₄HCO₃] and 100% [CH₃CN] in 1.6 min, then under this condition for 1.4 min, finally changed to 95% [water + 10 mM NH₄HCO₃] and 5% [CH₃CN] in 0.1 min and under this condition for 0.7 min.) Purity: 99.33%. Rt = 2.028 min; MS Calcd.: 678.3; MS Found: 679.2 [M + | ¹H NMR (400 MHz, DMSO-d₆) δ: 12.56 (s, 1H), 10.06 (s, 1H), 8.90 (d, J = 4.8 Hz, 1H), 8.21-8.04 (m, 3H), 8.02 (d, J = 1.6 Hz, 2H), 7.64-7.57 (m, 4H), 7.57-7.37 (m, 4H), 6.95 (s, 2H), 6.81 (s, 1H), 4.14 (s, 2H), 2.51 (s, 6H), 2.49-2.33 (m, 2H), 2.32-2.30 (m, 2H), 2.17 (s, 3H), 2.07-2.06 (m, 2H), 1.91 (s, 1H), 1.63- 1.58 (m, 1H), 1.50- 1.47 (m, 2H), 0.56- 0.52 (m, 2H). |
| | | H]⁺. | |
| I-76 | 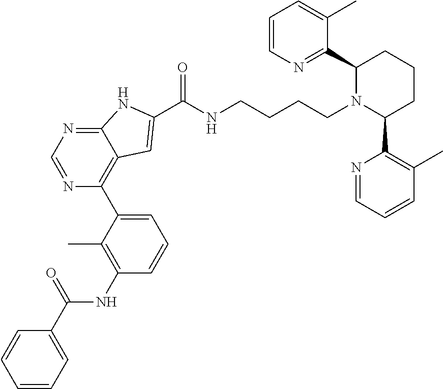 [SMILES: Cc1cccnc1[C@H]1CCC[C@@H](c2ncccc2C)N1CCCCNC(=O)c1cc2c(-c3cccc(NC(=O)c4ccccc4)c3C)ncnc2[nH]1] | LCMS (Agilent LCMS 1200-6120, Column: Waters X-Bridge C18 (50 mm*4.6 mm*3.5 μm); Column Temperature: 40° C.; Flow Rate: 2.0 mL/min; Mobile Phase: from 95% [water + 10 mM NH₄HCO₃] and 5% [CH₃CN] to 0% [water + 10 mM NH₄HCO₃] and 100% [CH₃CN] in 1.6 min, then under this condition for 1.4 min, finally changed to 95% [water + 10 mM NH₄HCO₃] and 5% [CH₃CN] in 0.1 min and under this condition for 0.7 min.) Purity: 98.11%. Rt = 2.093 min; MS Calcd.: 692.3; MS Found: 693.2 [M + | ¹H NMR (400 MHz, DMSO-d₆) δ: 12.63 (s, 1H), 10.06 (s, 1H), 8.91 (s, 1H), 8.29-8.26 (m, 3H), 8.02 (d, J = 7.2 Hz, 2H), 7.63-7.59 (m, 4H), 7.55-7.33 (m, 4H), 7.03 (s, 2H), 6.93 (s, 1H), 4.18 (s, 2H), 2.67-2.51 (m, 2H), 2.49 (s, 6H), 2.33-2.23 (m, 2H), 2.16 (s, 5H), 2.12- 2.06 (m, 1H), 1.98- 1.93 (m, 1H), 1.62- 1.45 (m, 2H), 0.66 (s, 2H), 0.29-0.22 (m, 2H). |
| | | H]⁺. | |
| I-77 | 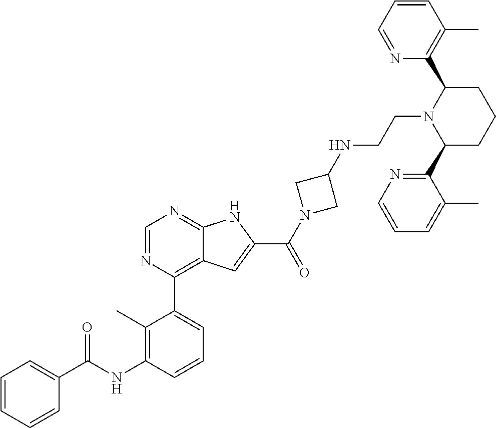 [SMILES: Cc1cccnc1[C@H]1CCC[C@@H](c2ncccc2C)N1CCNC1CN(C(=O)c2cc3c(-c4cccc(NC(=O)c5ccccc5)c4C)ncnc3[nH]2)C1] | LCMS (Agilent LCMS 1200-6120, Column: Waters X-Bridge C18 (50 mm*4.6 mm*3.5 μm); Column Temperature: 40° C.; Flow Rate: 2.0 mL/min; Mobile Phase: from 95% [water + 10 mM NH₄HCO₃] and 5% [CH₃CN] to 0% [water + 10 mM NH₄HCO₃] and 100% [CH₃CN] in 1.6 min, then under this condition for 1.4 min, finally changed to 95% [water + 10 mM NH₄HCO₃] and 5% [CH₃CN] in 0.1 min and under this condition for 0.7 min.) Purity: 100.00%. Rt = 1.909 min; MS Calcd.: 719.4; MS Found: 720.2 [M + H]⁺. | ¹H NMR (400 MHz, DMSO-d₆) δ: 12.71 (s, 1H), 10.11 (s, 1H), 8.95 (s, 1H), 8.31 (d, J = 5.2 Hz, 2H), 8.02 (d, J = 7.6 Hz, 2H), 7.64- 7.53 (m, 4H), 7.48-7.39 (m, 4H), 7.02 (t, J = 4.4 Hz, 2H), 6.43 (s, 1H), 4.23 (d, J = 9.6 Hz, 2H), 4.08 (t, J = 3.6 Hz, 1H), 3.82 (t, J = 8.4 Hz, 1H), 3.42 (s, 1H), 3.25 (s, 1H), 2.83 (s, 1H), 2.47 (s, 6H), 2.33 (s, 2H), 2.17 (s, 5H), 2.08- 1.95 (m, 1H), 1.67-1.60 (m, 1H), 1.47-1.44 (m, 3H), 1.24-1.07 (m, 2H). |
| I-78 | 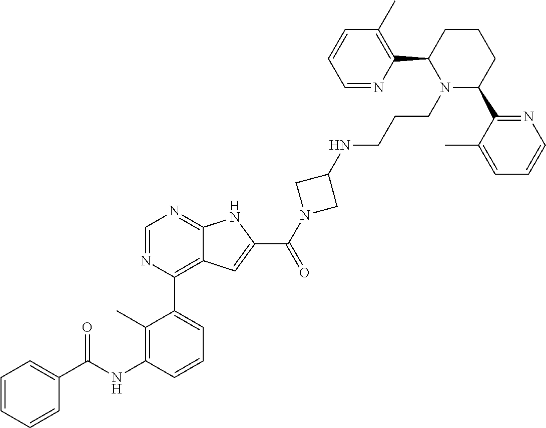 [SMILES: Cc1cccnc1[C@H]1CCC[C@@H](c2ncccc2C)N1CCCNC1CN(C(=O)c2cc3c(-c4cccc(NC(=O)c5ccccc5)c4C)ncnc3[nH]2)C1] | LCMS (Agilent LCMS 1200-6120, Column: Waters X-Bridge C18 (50 mm*4.6 mm*3.5 μm); Column Temperature: 40° C.; Flow Rate: 2.0 mL/min; Mobile Phase: from 95% [water + 10 mM NH₄HCO₃] and 5% [CH₃CN] to 0% [water + 10 mM NH₄HCO₃] and 100% [CH₃CN] in 1.6 min, then under this condition for 1.4 min, finally changed to 95% [water + 10 mM NH₄HCO₃] and 5% [CH₃CN] in 0.1 min and under this condition for 0.7 min.) Purity: 99.64%. Rt = 1.910 min; MS Calcd.: 733.4; MS Found: 734.2 [M + H]⁺. | ¹H NMR (400 MHz, DMSO-d₆) δ: 12.75 (s, 1H), 10.10 (s, 1H), 8.94 (s, 1H), 8.31 (s, 2H), 8.02 (d, J = 6.8 Hz, 2H), 7.64- 7.61 (m, 1H), 7.56- 7.52 (m, 3H), 7.48- 7.40 (m, 4H), 7.12- 7.05 (m, 2H), 6.51 (s, 1H), 4.32 (t, J = 7.6 Hz, 1H), 4.21 (d, J = 6.8 Hz, 2H), 3.96 (t, J = 9.2 Hz, 1H), 3.73 (s, 1H), 3.42 (t, J = 4.0 Hz, 1H), 3.09 (s, 1H), 2.51 (s, 6H), 2.33- 2.20 (m, 2H), 2.17 (s, 5H), 2.08-1.95 (m, 1H), 1.70-1.60 (m, 2H), 1.56-1.47 (m, 4H), 0.27-0.22 (m, 2H). |
| I-79 | 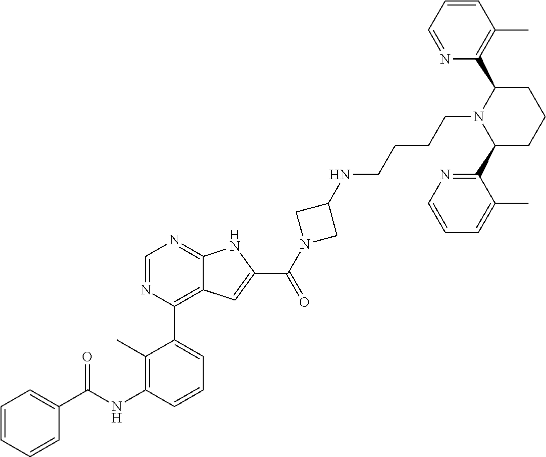 [SMILES: Cc1cccnc1[C@H]1CCC[C@@H](c2ncccc2C)N1CCCCNC1CN(C(=O)c2cc3c(-c4cccc(NC(=O)c5ccccc5)c4C)ncnc3[nH]2)C1] | LCMS (Agilent LCMS 1200-6120, Column: Waters X-Bridge C18 (50 mm*4.6 mm*3.5 μm); Column Temperature: 40° C.; Flow Rate: 2.0 mL/min; Mobile Phase: from 95% [water + 10 mM NH₄HCO₃] and 5% [CH₃CN] to 0% [water + 10 mM NH₄HCO₃] and 100% [CH₃CN] in 1.6 min, then under this condition for 1.4 min, finally changed to 95% [water + 10 mM NH₄HCO₃] and 5% [CH₃CN] in 0.1 min and under this condition for 0.7 min.) Purity: 99.05%. Rt = 1.976 min; MS Calcd.: 747.4; MS Found: 748.2 [M + H]⁺. | ¹H NMR (400 MHz, DMSO-d₆) δ: 12.65 (s, 1H), 10.10 (s, 1H), 8.94 (s, 1H), 8.31 (s, 2H), 8.01 (d, J = 7.2 Hz, 2H), 7.64- 7.52 (m, 4H), 7.48-7.40 (m, 4H), 7.11 (d, J = 2.8 Hz, 2H), 6.56 (s, 1H), 4.45 (t, J = 8.0 Hz, 1H), 4.12 (s, 2H), 4.08 (t, J = 9.2 Hz, 1H), 3.92- 3.89 (m, 1H), 3.58-3.54 (m, 1H), 3.28 (s, 1H), 2.51 (s, 6H), 2.17 (s, 7H), 2.00-1.90 (m, 2H), 1.83-1.76 (m, 2H), 1.63-1.57 (m, 1H), 1.49-1.46 (m, 2H), 0.53 (s, 2H), 0.28-0.22 (m, 2H). |
| I-80 | 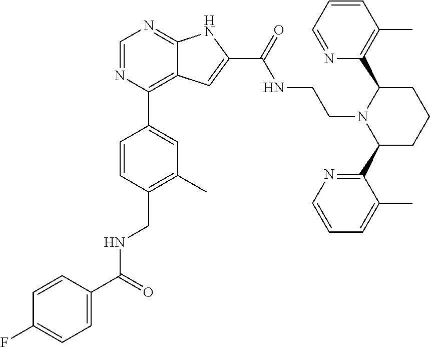 | LCMS (Agilent LCMS 1200-6120, Column: Waters X-Bridge C18 (50 mm*4.6 mm*3.5 μm); Column Temperature: 40° C.; Flow Rate: 2.0 mL/min; Mobile Phase: from 95% [water + 10 mM NH₄HCO₃] and 5% [CH₃CN] to 0% [water + 10 mM NH₄HCO₃] and 100% [CH₃CN] in 1.6 min, then under this condition for 1.4 min, finally changed to 95% [water + 10 mM NH₄HCO₃] and 5% [CH₃CN] in 0.1 min and under this condition for 0.7 min.) Purity: | ¹H NMR (400 MHz, DMSO-d₆) δ: 12.46 (s, 1H), 9.08 (t, J = 5.6 Hz, 1H), 8.84 (s, 1H), 8.31 (d, J = 4.4 Hz, 2H),8.03-8.00 (m, 2H), 7.94 (t, J = 12.0 Hz, 2H), 7.84 (t, J = 0.8 Hz, 1H), 7.43 (d, J = 8.4 Hz, 3H), 7.37-7.33 (m, 3H), 7.08 (t, J = 5.6 Hz, 1H), 4.55 (d, J = 5.6 Hz, 2H), 4.27 (t, J = 3.6 Hz, 2H), 2.55-2.51 (m, 8H), 2.45 (s, 3H), 2.15-2.08 (m, 4H), 1.96 (d, J = 11.6 Hz, 1H), 1.53 (d, |
| | | 100.00%. Rt = 1.913 | J = 13.6 Hz, 2H). |
| | | min; MS Calcd.: 696.3; | |
| | | MS Found: 697.2 [M + | |
| | | H]⁺. | |
| I-81 | 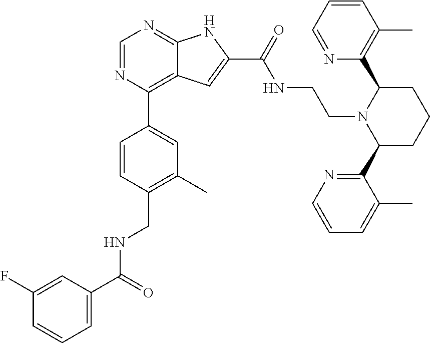 | LCMS (Agilent LCMS 1200-6120, Column: Waters X-Bridge C18 (50 mm*4.6 mm*3.5 μm); Column Temperature: 40° C.; Flow Rate: 2.0 mL/min; Mobile Phase: from 95% [water + 10 mM NH₄HCO₃] and 5% [CH₃CN] to 0% [water + 10 mM NH₄HCO₃] and 100% [CH₃CN] in 1.6 min, then under this condition for 1.4 min, finally changed to 95% [water + 10 mM NH₄HCO₃] and 5% [CH₃CN] in 0.1 min and under this condition for 0.7 min.) Purity: | ¹H NMR (400 MHz, DMSO-d₆) δ: 12.47 (s, 1H), 9.15 (t, J = 5.6 Hz, 1H), 8.84 (s, 1H), 8.31 (d, J = 4.0 Hz, 2H), 7.94 (t, J = 10.8 Hz, 2H), 7.81 (s, 1H), 7.81-7.75 (m, 1H), 7.75-7.72 (m, 1H), 7.60-7.55 (s, 1H), 7.46-7.41 (m, 4H), 7.38 (s, 4H), 7.08 (t, J = 5.2 Hz, 2H), 7.46 (d, J = 5.6 Hz, 2H), 4.26 (d, J = 8.0 Hz, 2H), 2.55-2.47 (m, 8H), 2.45 (s, 3H), 2.13-2.07 (m, 4H), 1.95 (s, 1H), 1.56- |
| | | 97.64%. Rt = 1.928 | 1.54 (m, 1H), 1.53 |
| | | min; MS Calcd.: 696.2; | (d, J = 11.6 Hz, |
| | | MS Found: 697.3 [M + | 2H). |
| | | H]⁺. | |
| I-82 | 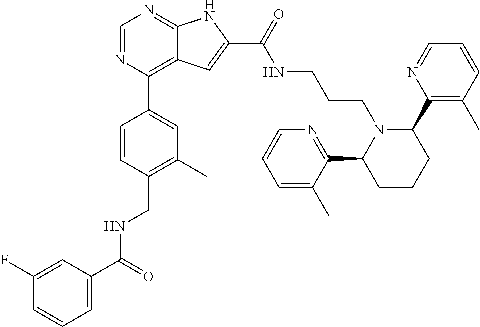 | LCMS (Agilent LCMS 1200-6120, Column: Waters X-Bridge C18 (50 mm*4.6 mm*3.5 μm); Column Temperature: 40° C.; Flow Rate: 2.0 mL/min; Mobile Phase: from 95% [water + 10 mM NH₄HCO₃] and 5% [CH₃CN] to 0% [water + 10 mM NH₄HCO₃] and 100% [CH₃CN] in 1.6 min, then under this condition for 1.4 min, finally changed to 95% [water + 10 mM NH₄HCO₃] and 5% [CH₃CN] in 0.1 min and under this condition for | ¹H NMR (400 MHz, DMSO-d₆) δ: 12.53 (s, 1H), 9.14 (t, J = 5.6 Hz, 1H), 8.84 (s, 1H), 8.19 (s, 3H), 7.94 (t, J = 8.0 Hz, 2H), 7.79-7.71 (m, 2H), 7.57-7.52 (m, 1H), 7.45-7.35 (m, 5H), 6.93 (s, 2H), 4.55 (d, J = 5.6 Hz, 2H), 4.11 (s, 2H), 2.49- 2.46 (m, 9H), 2.30 (s, 2H), 2.04 (t, J = 2.4 Hz, 2H), 1.93- 1.87 (m, 1H), 1.58- 1.42 (m, 3H), 0.59- 0.46 (m, 2H). |
| | | 0.7 min.) Purity: | |
| | | 96.96%. Rt = 1.798 | |
| | | min; MS Calcd.: 710.4; | |
| | | MS Found: 711.3 [M + | |
| | | H]⁺. | |
| I-83 | 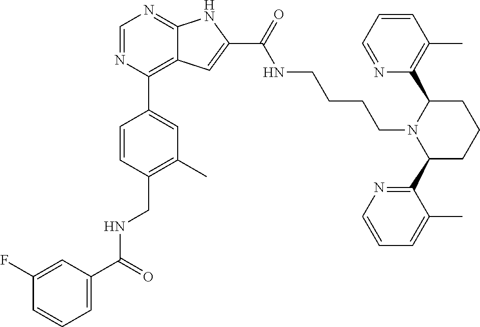 | LCMS (Agilent LCMS 1200-6120, Column: Waters X-Bridge C18 (50 mm*4.6 mm*3.5 μm); Column Temperature: 40° C.; Flow Rate: 2.0 mL/min; Mobile Phase: from 95% [water + 10 mM NH₄HCO₃] and 5% [CH₃CN] to 0% [water + 10 mM NH₄HCO₃] and 100% [CH₃CN] in 1.6 min, then under this condition for 1.4 min, finally changed to 95% [water + 10 mM NH₄HCO₃] and 5% [CH₃CN] in 0.1 min and under this condition for | ¹H NMR (400 MHz, DMSO-d₆) δ: 12.62 (s, 1H), 9.17 (t, J = 6.0 Hz, 1H), 8.87 (s, 1H), 8.30 (s, 3H), 7.98 (t, J = 7.6 Hz, 2H), 7.82-7.75 (m, 1H), 7.74-7.73 (m, 1H), 7.60-7.55 (m, 1H), 7.48-7.38 (m, 5H), 7.03 (s, 2H), 7.57 (d, J = 5.6 Hz, 2H), 4.18 (s, 2H), 4.67 (t, J = 1.2 Hz, 2H), 2.62 (s, 3H), 2.51 (s, 6H), 2.26 (s, 2H), 2.15-2.08 (m, 2H), 1.65-1.52 (m, 3H), 0.54 (s, 2H), |
| | | 0.7 min.) Purity: | 0.53-0.12 (m, 2H). |
| | | 98.00%. Rt = 2.040 | |
| | | min; MS Calcd.: 724.4; | |
| | | MS Found: 725.2 [M + | |
| | | H]⁺. | |
| I-84 | 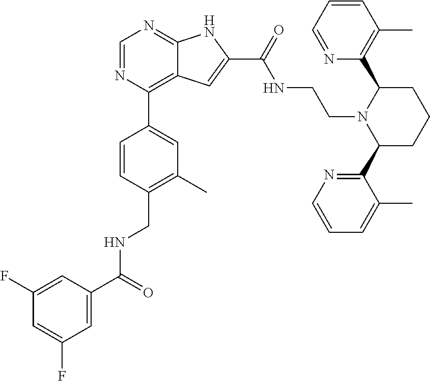 [SMILES: Cc1cc(-c2ncnc3[nH]c(C(=O)NCCN4[C@@H](c5ncccc5C)CCC[C@H]4c4ncccc4C)cc23)ccc1CNC(=O)c1cc(F)cc(F)c1] | LCMS (Agilent LCMS 1200-6120, Column: Waters X-Bridge C18 (50 mm*4.6 mm*3.5 μm); Column Temperature: 40° C.; Flow Rate: 2.0 mL/min; Mobile Phase: from 95% [water + 10 mM NH₄HCO₃] and 5% [CH₃CN] to 0% [water + 10 mM NH₄HCO₃] and 100% [CH₃CN] in 1.6 min, then under this condition for 1.4 min, finally changed to 95% [water + 10 mM NH₄HCO₃] and 5% [CH₃CN] in 0.1 min and under this condition for 0.7 min.) Purity: 100.00%. Rt = 2.193 min; MS Calcd.: 713.3; | ¹H NMR (400 MHz, DMSO-d₆) δ: 12.48 (s, 1H), 9.21 (t, J = 5.6 Hz, 1H), 8.84 (s, 1H), 8.31 (d, J = 3.6 Hz, 2H), 7.97-7.90 (m, 2H), 7.86 (s, 1H), 7.68-7.63 (m, 2H), 7.54-7.44 (m, 4H), 7.36 (d, J = 6.8 Hz, 1H), 7.08 (t, J = 5.6 Hz, 2H), 4.56 (d, J = 5.6 Hz, 2H), 4.26 (d, J = 9.2 Hz, 2H), 2.67 (d, J = 1.6 Hz, 8H), 2.45 (s, 3H), 2.13-1.89 (m, 5H), 1.76-1.62 (m, 1H), 1.54 (d, J = 13.2 Hz, 2H). |
| | | MS Found: 715.1 [M + | |
| | | H]⁺. | |
| I-85 | 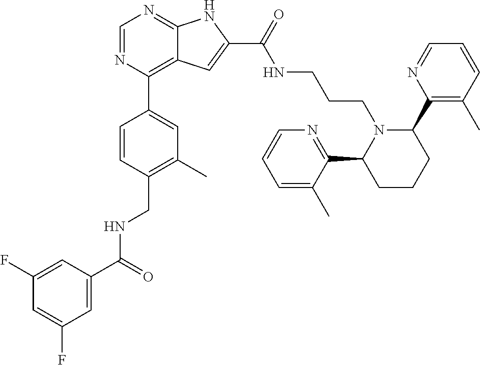 | LCMS (Agilent LCMS 1200-6120, Column: Waters X-Bridge C18 (50 mm*4.6 mm*3.5 μm); Column Temperature: 40° C.; Flow Rate: 2.0 mL/min; Mobile Phase: from 95% [water + 10 mM NH₄HCO₃] and 5% [CH₃CN] to 0% [water + 10 mM NH₄HCO₃] and 100% [CH₃CN] in 1.6 min, then under this condition for 1.4 min, finally changed to 95% [water + 10 mM NH₄HCO₃] and 5% [CH₃CN] in 0.1 min and under this condition for 0.7 min.) Purity: 100.00%. Rt = 2.079 | ¹H NMR (400 MHz, DMSO-d₆) δ: 12.55 (s, 1H), 9.23 (t, J = 5.6 Hz, 1H), 8.86 (d, J = 4.8 Hz, 1H), 8.22 (s, 3H), 7.99-7.95 (m, 2H), 7.68-7.63 (m, 2H), 7.54-7.38 (m, 5H), 6.96 (s, 2H), 4.58 (d, J = 5.2 Hz, 2H), 4.14 (s, 2H), 2.68- 2.60 (m, 8H), 2.48 (s, 3H), 2.34-2.33 (m, 2H), 1.15-1.89 (m, 3H), 1.65-1.50 (m, 3H), 0.63-0.53 (m, 2H). |
| | | min; MS Calcd.: 728.3; | |
| | | MS Found: 729.2 [M + | |
| | | H]⁺. | |
| I-86 | 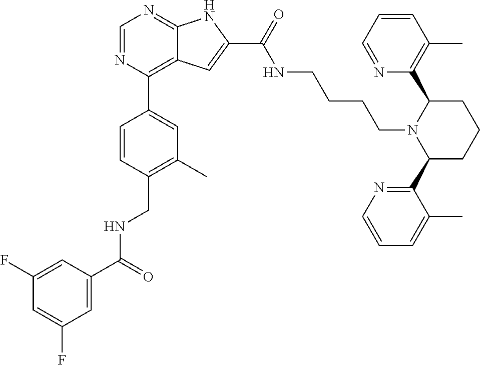 | LCMS (Agilent LCMS 1200-6120, Column: Waters X-Bridge C18 (50 mm*4.6 mm*3.5 μm); Column Temperature: 40° C.; Flow Rate: 2.0 mL/min; Mobile Phase: from 95% [water + 10 mM NH₄HCO₃] and 5% [CH₃CN] to 0% [water + 10 mM NH₄HCO₃] and 100% [CH₃CN] in 1.6 min, then under this condition for 1.4 min, finally changed to 95% [water + 10 mM NH₄HCO₃] and 5% [CH₃CN] in 0.1 min and under this condition for 0.7 min.) Purity: 99.35%. Rt = 1.895 | ¹H NMR (400 MHz, DMSO-d₆) δ: 12.62 (s, 1H), 9.24 (t, J = 5.6 Hz, 1H), 8.88 (s, 1H), 8.30 (s, 3H), 8.00- 7.96 (m, 2H), 7.68- 7.64 (m, 2H), 7.54- 7.47 (m, 5H), 7.39 (d, J = 4.8 Hz, 2H), 4.58 (d, J = 5.2 Hz, 2H), 4.18 (d, J = 4.8 Hz, 2H), 2.68 (t, J = 5.6 Hz, 3H), 2.62 (t, J = 2.4 Hz, 3H), 2.49 (s, 6H), 2.26 (m, 1H), 2.08 (t, J = 3.2 Hz, 2H), 1.92 (d, J = 15.2 Hz, 1H), 1.64-1.46 (m, 3H), 0.67 (s, 2H), (s, 2H). |
| | | min; MS Calcd.: 742.4; | |
| | | MS Found: 743.3 [M + | |
| | | H]⁺. | |
| I-87 | 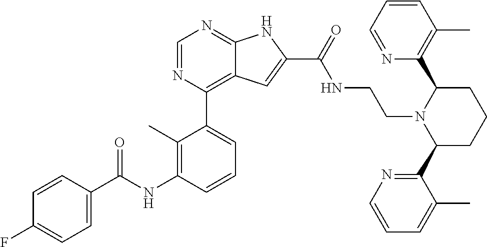 | LCMS (Agilent LCMS 1200-6120, Column: Waters X-Bridge C18 (50 mm*4.6 mm*3.5 μm); Column Temperature: 40° C.; Flow Rate: 2.0 mL/min; Mobile Phase: from 95% [water + 10 mM NH₄HCO₃] and 5% [CH₃CN] to 0% [water + 10 mM NH₄HCO₃] and 100% [CH₃CN] in 1.6 min, then under this condition for 1.4 min, | ¹H NMR (400 MHz, DMSO-d₆) δ: 12.47 (s, 1H), 10.07 (s, 1H), 8.87 (s, 1H), 8.30 (d, J = 3.6 Hz, 2H), 8.11- 8.07 (m, 2H), 7.74 (s, 1H), 7.51 (d, J = 7.6 Hz, 1H), 7.45- 7.34 (m, 6H), 7.08 (t, J = 4.8 Hz, 2H), 6.81 (s, 1H), 4.26 (d, J = 6.8 Hz, 2H), 2.54 (s, 6H), 2.44 (s, 2H), 2.11 (s, |
| | | finally changed to 95% | 7H), 1.96 (d, J = |
| | | [water + 10 mM | 7.2 Hz, 1H), 1.68- |
| | | NH₄HCO₃] and 5% | 1.65 (m, 1H), 1.51 |
| | | [CH₃CN] in 0.1 min and | (d, J = 11.6 Hz, |
| | | under this condition for | 2H). |
| | | 0.7 min.) Purity: | |
| | | 100.00%. Rt = 2.108 | |
| | | min; MS Calcd.: 682.3; | |
| | | MS Found: 683.2 [M + | |
| | | H]⁺. | |
| I-88 | 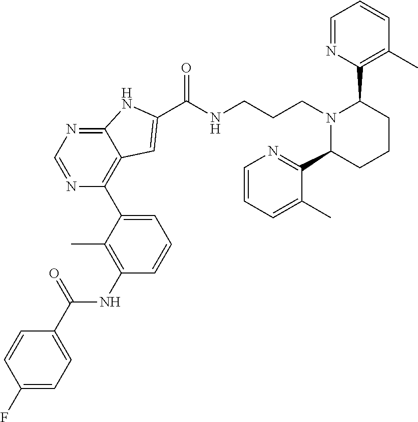 [SMILES: Cc1cccnc1[C@H]1CCC[C@@H](c2ncccc2C)N1CCCNC(=O)c1cc2c(-c3cccc(NC(=O)c4ccc(F)cc4)c3C)ncnc2[nH]1] | LCMS (Agilent LCMS 1200-6120, Column: Waters X-Bridge C18 (50 mm*4.6 mm*3.5 μm); Column Temperature: 40° C.; Flow Rate: 2.0 mL/min; Mobile Phase: from 95% [water + 10 mM NH₄HCO₃] and 5% [CH₃CN] to 0% [water + 10 mM NH₄HCO₃] and 100% [CH₃CN] in 1.6 min, then under this condition for 1.4 min, finally changed to 95% [water + 10 mM NH₄HCO₃] and 5% [CH₃CN] in 0.1 min and under this condition for 0.7 min.) Purity: 100.00%. Rt = 1.985 min; MS Calcd.: 696.3; MS Found: 697.2 [M + H]⁺. | ¹H NMR (400 MHz, DMSO-d₆) δ: 12.56 (s, 1H), 10.09 (s, 1H), 8.90 (t, J = 5.2 Hz, 1H), 8.21 (s, 2H), 8.12- 8.08 (m, 3H), 7.53 (d, J = 7.6 Hz, 1H), 7.45-7.37 (m, 6H), 6.95 (s, 2H), 6.81 (s, 1H), 4.14 (t, J = 5.2 Hz, 2H), 2.67 (s, 6H), 2.40 (d, J = 2.0 Hz, 2H), 2.33- 2.20 (m, 2H), 2.16 (s, 3H), 2.08-2.00 (m, 2H), 1.98-1.91 (m, 1H), 1.63-1.55 (m, 1H), 1.48 (d, J = 10.0 Hz, 2H), 0.57 (d, J = 2.4 Hz, 2H). |
| I-89 | 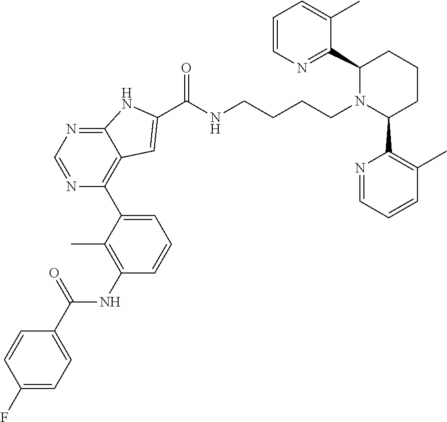 [SMILES: Cc1cccnc1[C@H]1CCC[C@@H](c2ncccc2C)N1CCCCNC(=O)c1cc2c(-c3cccc(NC(=O)c4ccc(F)cc4)c3C)ncnc2[nH]1] | LCMS (Agilent LCMS 1200-6120, Column: Waters X-Bridge C18 (50 mm*4.6 mm*3.5 μm); Column Temperature: 40° C.; Flow Rate: 2.0 mL/min; Mobile Phase: from 95% [water + 10 mM NH₄HCO₃] and 5% [CH₃CN] to 0% [water + 10 mM NH₄HCO₃] and 100% [CH₃CN] in 1.6 min, then under this condition for 1.4 min, finally changed to 95% [water + 10 mM NH₄HCO₃] and 5% [CH₃CN] in 0.1 min and under this condition for 0.7 min.) Purity: 100.00%. Rt = 2.026 min; MS Calcd.: 710.3; MS Found: 711.2 [M + H]⁺. | ¹H NMR (400 MHz, DMSO-d₆) δ: 12.64 (s, 1H), 10.09 (s, 1H), 8.91 (t, J = 5.2 Hz, 1H), 8.27 (d, J = 14.8 Hz, 3H), 8.11-8.08 (m, 2H), 7.54-7.52 (m, 1H), 7.45-7.37 (m, 6H), 7.03 (s, 2H), 6.93 (s, 1H), 4.17 (s, 2H), 2.72 (s, 2H), 2.62 (s, 6H), 2.40 (s, 2H), 2.15 (s, 5H), 1.99-1.93 (m, 1H), 1.63-1.55 (m, 1H), 1.47 (d, J = 8.8 Hz, 2H), 0.71- 0.56 (m, 2H), 0.32-0.26 (m, 2H). |
| I-90 | 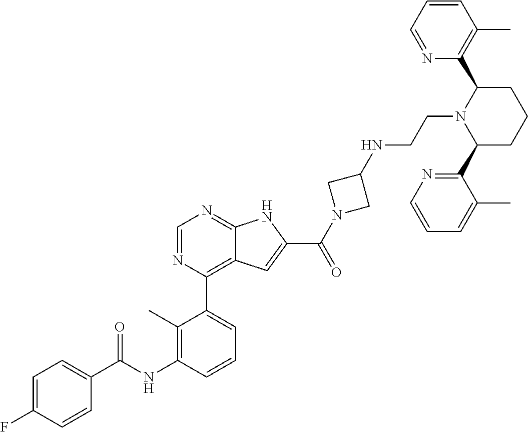 [SMILES: Cc1cccnc1[C@H]1CCC[C@@H](c2ncccc2C)N1CCNC1CN(C(=O)c2cc3c(-c4cccc(NC(=O)c5ccc(F)cc5)c4C)ncnc3[nH]2)C1] | LCMS (Agilent LCMS 1200-6120, Column: Waters X-Bridge C18 (50 mm*4.6 mm*3.5 μm); Column Temperature: 40° C.; Flow Rate: 2.0 mL/min; Mobile Phase: from 95% [water + 10 mM NH₄HCO₃] and 5% [CH₃CN] to 0% [water + 10 mM NH₄HCO₃] and 100% [CH₃CN] in 1.6 min, then under this condition for 1.4 min, finally changed to 95% [water + 10 mM NH₄HCO₃] and 5% [CH₃CN] in 0.1 min and under this condition for 0.7 min.) Purity: 100.00%. Rt = 1.962 min; MS Calcd.: 737.4; MS Found: 738.2 [M + H]⁺. | ¹H NMR (400 MHz, DMSO-d₆) δ: 12.71 (s, 1H), 10.14 (s, 1H), 8.96 (s, 1H), 8.30 (d, J = 5.6 Hz, 2H), 8.11-8.07 (s, 2H), 7.56-7.53 (m, 1H), 7.48-7.44 (m, 3H), 7.41-7.31 (m, 3H), 7.02 (t, J = 4.8 Hz, 2H), 6.43 (s, 1H), 4.22 (d, J = 5.6 Hz, 2H), 4.08 (t, J = 4.4 Hz, 1H), 3.82 (t, J = 8.4 Hz, 1H), 3.51-3.43 (m, 1H), 3.29-3.24 (m, 1H), 2.84-2.80 (m, 1H), 2.46 (s, 6H), 2.39 (d, J = 6.0 Hz, 2H), 2.16 (s, 5H), 1.98-1.95 (m, 1H), 1.67-1.61 (m, 1H), 1.38 (d, J = 2.0 Hz, 3H), 1.23- |
| | | | 1.08 (m, 2H). |
| I-91 | 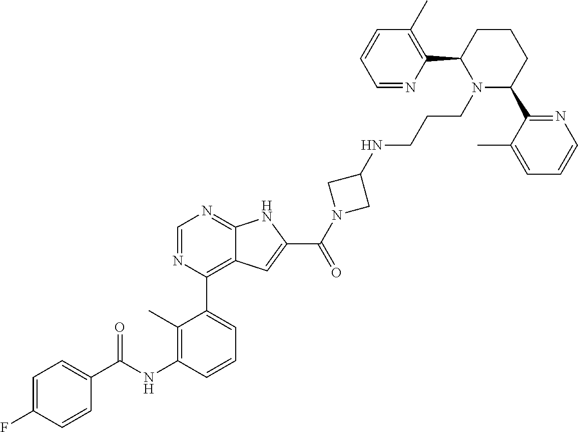 [SMILES: Cc1cccnc1[C@H]1CCC[C@@H](c2ncccc2C)N1CCCNC1CN(C(=O)c2cc3c(-c4cccc(NC(=O)c5ccc(F)cc5)c4C)ncnc3[nH]2)C1] | LCMS (Agilent LCMS 1200-6120, Column: Waters X-Bridge C18 (50 mm*4.6 mm*3.5 μm); Column Temperature: 40° C.; Flow Rate: 2.0 mL/min; Mobile Phase: from 95% [water + 10 mM NH₄HCO₃] and 5% [CH₃CN] to 0% [water + 10 mM NH₄HCO₃] and 100% [CH₃CN] in 1.6 min, then under this condition for 1.4 min, finally changed to 95% [water + 10 mM NH₄HCO₃] and 5% [CH₃CN] in 0.1 min and under this condition for 0.7 min.) Purity: 100.00%. Rt = 1.975 min; MS Calcd.: 751.4; MS Found: 752.1 [M + H]⁺. | ¹H NMR (400 MHz, DMSO-d₆) δ: 10.13 (s, 1H), 8.94 (s, 1H), 8.31 (s, 2H), 8.11-8.07 (s, 2H), 7.55-7.35 (m, 7H), 7.12-7.07 (m, 2H), 6.51 (s, 1H), 4.31 (t, J = 8.0 Hz, 1H), 4.20 (s, 2H), 3.96 (t, J = 9.2 Hz, 1H), 3.73 (s, 1H), 3.42 (t, J = 5.6 Hz, 1H), 3.09 (s, 1H), 2.51 (s, 6H), 2.33-2.30 (m, 2H), 2.16 (s, 5H), 2.02-1.95 (m, 2H), 1.66-1.47 (m, 6H), 0.31-0.24 (m, 2H). |
| I-92 | 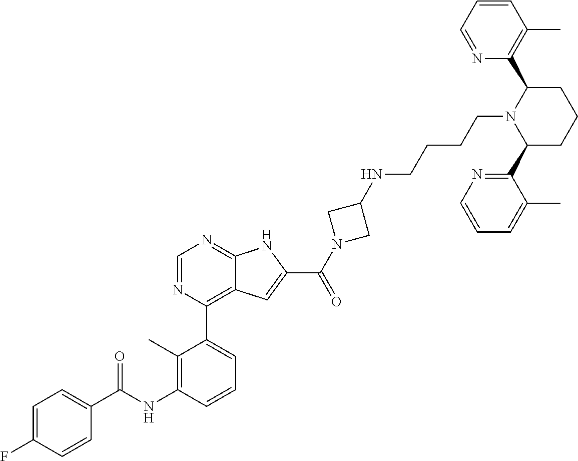 [SMILES: Cc1cccnc1[C@H]1CCC[C@@H](c2ncccc2C)N1CCCCNC1CN(C(=O)c2cc3c(-c4cccc(NC(=O)c5ccc(F)cc5)c4C)ncnc3[nH]2)C1] | LCMS (Agilent LCMS 1200-6120, Column: Waters X-Bridge C18 (50 mm*4.6 mm*3.5 μm) ; Column Temperature: 40° C.; Flow Rate: 2.0 mL/min; Mobile Phase: from 95% [water + 10 mM NH₄HCO₃] and 5% [CH₃CN] to 0% [water + 10 mM NH₄HCO₃] and 100% [CH₃CN] in 1.6 min, then under this condition for 1.4 min, finally changed to 95% [water + 10 mM NH₄HCO₃] and 5% [CH₃CN] in 0.1 min and under this condition for 0.7 min.) Purity: 100.00%. Rt = 1.996 min; MS Calcd.: 765.4; MS Found: 766.2 [M + H]⁺. | ¹H NMR (400 MHz, DMSO-d₆) δ: 10.13 (s, 1H), 8.94 (s, 1H), 8.31 (s, 2H), 8.10-8.07 (m, 2H), 7.54-7.35 (m, 7H), 7.11 (d, J = 3.2 Hz, 2H), 6.57 (s, 1H), 4.45 (t, J = 8.0 Hz, 1H), 4.17 (d, J = 5.6 Hz, 1H), 4.08 (t, J = 8.4 Hz, 2H), 3.94-3.86 (m, 1H), 3.58-3.54 (m, 1H), 2.51 (s, 6H), 2.18- 2.00 (m, 8H), 1.93-1.82 (m, 2H), 1.76 (s, 3H), 1.70- 1.60 (m, 1H), 1.55-1.46 (m, 2H), 0.53 (s, 2H), 0.34- 0.15 (m, 2H). |

### Example 19: CXCR4 Screening Assays

### Intracellular CXCL-12-Induced Calcium Mobilization Assay

Intracellular calcium mobilization induced by chemokines or chemokine-derived peptides were evaluated using a calcium responsive fluorescent probe and a FLIPR system. The CXCR-4 transfected U87 cell line (U87.CXCR4) cells were seeded in gelatine-coated black-wall 96-well plates at 20,000 cells per well and incubated for 12 hours. Cells were then loaded with the fluorescent calcium probe Fluo-2 acetoxymethyl at 4 μM final concentration in assay buffer (Hanks' balanced salt solution with 20 mM HEPES buffer and 0.2% bovine serum albumin, pH 7.4) for 45 min at 37° C. The intracellular calcium mobilization induced by the CXCL-12 (25-50 ng/ml) was then measured at 37° C. by monitoring the fluorescence as a function of time in all the wells simultaneously using a fluorometric imaging plate reader (FLIPR Tetra, Molecular Devices). The test compounds were added 15 minutes before the addition of CXCL-12 and monitored to see if compounds induced signals by themselves (agonistic properties).

### Chemokine (CXCL12-AF647) Binding Inhibition Assay

Jurkat cells expressing CXCR4 were washed once with assay buffer (Hanks' balanced salt solution with 20 mM HEPES buffer and 0.2% bovine serum albumin, pH 7.4) and then incubated for 15 min at room temperature with the test compounds diluted in assay buffer at dose-dependent concentrations. Subsequently, CXCL12-AF647 (25 ng/ml) was added to the compound-incubated cells. The cells were incubated for 30 min at room temperature. Thereafter, the cells were washed twice in assay buffer, fixed in 1% paraformaldehyde in PBS, and analyzed on the FL4 channel of a FACSCalibur flow cytometer equipped with a 635-nm red diode laser (Becton Dickinson, San Jose, CA, USA).

The percentages of inhibition of CXCL12-AF647 binding were calculated according to the formula: [1−((MFI−MFI_{NC})/(MFI_{PC}−MFI_{NC}))]×100 where MFI is the mean fluorescence intensity of the cells incubated with CXCL 12-AF647 in the presence of the inhibitor, MFI_{NC} is the mean fluorescence intensity measured in the negative control (i.e., autofluorescence of unlabeled cells), and MFI_{PC} is the mean fluorescence intensity of the positive control (i.e., cells exposed to CXCL12-AF647 alone).

### Results of Assays

Table 3 shows the activity of selected compounds of this invention in the assays described above. The compound numbers correspond to the compound numbers in Table 1. Compounds having an activity designated as “A” provided an IC₅₀ of 1 to 100 nM; compounds having an activity designated as “B” provided an IC₅₀ of >100 nm to <1 μM; compounds having an activity designated as “C” provided an IC₅₀ of 1 μM to <2.5 μM; and compounds having an activity designated as “D” provided an IC₅₀ of 2.5 μM or greater.

TABLE 3
Inhibition of Ca²⁺ Signaling and Inhibition of CXCL12 Binding

| | IC₅₀ CXCL- | IC₅₀ |
|---|---|---|
| | 12 Ca2⁺ flux | CXCL-12 |
| Compound | U87.CXCR4+ | binding |
| No. | (nM) | Jurkat (nM |
| I-1 | D | A |
| I-2 | D | A |
| I-3 | D | B |
| I-4 | D | A |
| I-5 | D | B |
| I-6 | D | A |
| I-7 | D | B |
| I-8 | B | A |
| I-9 | A | A |
| I-10 | A | A |
| I-11 | B | A |
| I-12 | C | A |
| I-13 | D | B |
| I-14 | D | D |
| I-15 | D | C |
| I-16 | B | A |
| I-17 | D | C |
| I-18 | D | B |
| I-20 | D | B |
| I-21 | B | A |
| I-22 | B | A |
| I-23 | | |
| I-24 | | |
| I-25 | | |
| I-26 | D | D |
| I-27 | D | B |
| I-28 | D | B |
| I-29 | D | |
| I-30 | A | A |
| I-31 | A | A |
| I-32 | D | A |
| I-33 | B | A |
| I-34 | B | A |
| I-35 | A | A |
| I-36 | B | A |
| I-37 | A | A |
| I-38 | B | A |
| I-39 | D | A |
| I-40 | D | A |
| I-41 | D | A |
| I-42 | B | A |
| I-43 | B | A |
| I-44 | B | A |
| I-45 | C | A |
| I-46 | B | A |
| I-47 | | |
| I-48 | | |
| I-49 | | |
| I-50 | | |
| I-51 | | |
| I-52 | | |
| I-53 | | |
| I-54 | | |
| I-55 | | |
| 1-56 | | |
| I-57 | | |
| I-58 | | |
| I-59 | | |
| I-60 | | |
| I-61 | D | D |
| 1-62 | | |
| I-63 | D | C |
| I-64 | D | D |
| I-65 | | |
| I-66 | D | D |

### Example 20: BTK Screening Assay (Enzyme Assay)

Inhibition of BTK activity was evaluated using the BTK Kinase Enzyme System in combination with the ADP-Glo™ Kinase Assay Kit (V9071, Promega), according to manufacturer's instructions. Luminescence was detected on a PerkinElmer EnVision Multimode Plate Reader.

Recombinant full-length human BTK was expressed by baculovirus in Sf9 insect cells using an N-terminal His tag, MW ˜78 kDa. Substrate solution contained Poly (4:1 Glu, Tyr) Peptide Substrate. BTK Kinase Buffer consisted of: 40 mM Tris, pH 7.5; 20 mM MgCl₂; 0.1 mg/ml BSA; 2 mM MnCl₂; 50 μM DTT. ADP-Glo™ Reagents were thawed at ambient temperature; components of BTK Enzyme System, ADP and ATP were thawed on ice. Final assay conditions used 3 ng BTK, 50 μM ATP, and 0.2 mg/mL substrate. Compounds were tested at 11 dose points in duplicates, in 3-fold dilution with top concentration at 10 μM.

IC₅₀ values were calculated as Transfer signal to inhibition %: (AVG0% inhibition−Signal)/(AVG0% inhibition−AVG100% inhibition)×100% Calculate IC₅₀ by standard 4-parameter fit method (Model 205, XL-fit). Assay performance was evaluated according to the reference compound Staurosporine, which was within 3-5 fold of published results and between different tests, with a Z′ value larger than 0.5, where applicable.

### Results of Assays

Table 4 shows the activity of selected compounds of this invention in the assays described above. The compound numbers correspond to the compound numbers in Table 1. Compounds having an activity designated as “A” provided an IC₅₀ of 1 to 100 M; compounds having an activity designated as “B” provided an IC₅₀ of >100 nm to <1 μM; compounds having an activity designated as “C” provided an IC₅₀ of 1 μM to <2.5 μM; and compounds having an activity designated as “D” provided an IC₅₀ of 2.5 μM or greater.

TABLE 4
Inhibition of BTK

| Compound | |
|---|---|
| No. | BTK IC₅₀ |
| I-1 | B |
| I-2 | C |
| I-3 | A |
| I-4 | A |
| I-5 | B |
| I-6 | A |
| I-7 | A |
| I-8 | A |
| I-9 | B |
| I-10 | A |
| I-11 | A |
| I-12 | A |
| I-13 | B |
| I-14 | A |
| I-15 | C |
| I-16 | A |
| I-17 | A |
| I-18 | A |

### Example 21: BTK Screening Assay (Ramos Cell Line Assay)

Inhibition of non-Hodgkin lymphoma B-cell proliferation by compounds of the present invention was evaluated in a Ramos cell line. Ramos is a B lymphocyte cell line that was derived from a patient with Burkitt's Lymphoma. This cell line has a high level of CXCR4 expression, and BTK inhibition has been shown to inhibit cell proliferation (Ibrutinib IC₅₀=0.87 μM; Chu et al. 2018).

Cell viability was analyzed with the CellTiter-Glo® Luminescent Cell Viability Assay Kit (G7572, Promega), according to the manufacturer's instruction. Cells were seeded into a 96-well/384-well plate and then incubated in 5% CO₂ at 37° C. overnight. Compounds were added to the cells, with 9-10 dose points in duplicates in 3-fold dilution from a top concentration of 30 μM, and incubated in 5% CO₂ at 37° C. for 72 hr. The 96-well/384-well plate was equilibrated at room temperature for 30 min. 50 μl/25 μl of CellTiter-Glo® Reagent was added to each well and mixed for 2 min on an orbital shaker to induce cell lysis. The 96-well/384-well plate was incubated at room temperature for 10 min to stabilize luminescent signal. Luminescence was detected on a PerkinElmer EnVision Multimode Plate Reader as relative luminescence units (RLU).

IC₅₀ values were calculated by standard 4-parameter fit method (Model 205, XL-fit), according to the following formula for percent inhibition: ((1−(RLU compound−AVG RLU 100% inhibition)/(AVG RLU 0% inhibition−AVG RLU 100% inhibition))*100%. Assay performance was evaluated according to the reference compound Staurosporine, which was within 3-fold of published results and between different tests, with a Z′ value larger than 0.5, where applicable.

Table 5 shows the activity of selected compounds of this invention in the assays described above. The compound numbers correspond to the compound numbers in Table 1. Compounds having an activity designated as “A” provided an IC₅₀ of 1 μM to <1.5 μM; compounds having an activity designated as “B” provided an IC₅₀ of 1.5 μM to <2 μM; compounds having an activity designated as “C” provided an IC₅₀ of 2 μM to <4 μM; and compounds having an activity designated as “D” provided an IC₅₀ of 4 μM or greater.

TABLE 5
Cell Viability IC₅₀'s in Ramos Cell Lines

| Compound | | Ramos |
|---|---|---|
| No. | IC₅₀ (uM) | viability IC₅₀ |
| Ibrutinib | 1.74 | B |
| I-6 | 1.55 | B |
| I-10 | 2.67 | C |
| 1-11 | 1.71 | B |
| I-12 | 2.78 | C |
| I-16 | 1.48 | A |
| I-18 | 1.33 | A |
| I-7 | 1.62 | B |
| I-8 | 11.10 | D |
| I-9 | 3.54 | C |
| Zanubrutinib | 11.87 | D |

### Example 22: Glioblastoma Multiforme (GBM) Cell Proliferation Assays

Inhibition of glioblastoma multiforme cell proliferation by compounds of the present invention was evaluated in U-87 MG (ATCC; Cat. No. HTB-14; Lot. No. 5018014) and T98G (ATCC; Cat. No. CRL-1690; Lot. No. 58267513) cell lines. These are adherent cell lines isolated from glioblastoma multiforme/malignant glioma, which express both CXCR4 and BTK (see Zhou et al. 2002 and Yue et al. 2017).

Cell viability was analyzed with the CellTiter Glo Cell assay kit (Corning, Cat. No. G7573, Lot. No. 0000474103), according to the manufacturer's instruction.

U-87 MG and T98G cells were seeded onto 384-well opaque-walled clear bottom plates (Corning, Cat. No. 3765, Lot. No. 34319047) in EMEM (Invitrogen, Cat. No. 11095-080; Lot. No. 2276656) supplemented with 10% FBS (Invitrogen, Cat. No. 10099-141C, Lot. No. 2145169CP), at a seeding density of 1000 or 1200 cells per well, respectively, and then incubated in 5% CO₂ at 37° C. overnight.

Compounds were prepared as stock solutions of 30 mM in DMSO (Sigma, Cat. No. 276855-1L. Lot. No. SHBL5610). Compounds were added to the cells, with 10 dose points in duplicates, in 3-fold dilution from a top concentration of 30 μM (final DMSO concentration 0.2%), and incubated in 5% CO₂ at 37° C. for 96 hr.

The 384-well plates were equilibrated at room temperature for approximately 30 min. 25 μl of CellTiter-Glo® Reagent was added to each well and mixed for 10 min on an orbital shaker to induce cell lysis. The 384-well plates were incubated at room temperature for 10 min to stabilize luminescent signal. Luminescence was detected on a PerkinElmer EnVision Multimode Plate Reader (measurement time 0.1s) as relative luminescence units (RLU).

IC₅₀s were calculated with XLFit curve fitting software using 4 Parameter Logistic Model [fit=(A+((B−A)/(1+((C/x)\hat{ }D))))], according to the following formula for percent inhibition: ((1−(RLU compound−AVGRLU 100% inhibition)/(AVGRLU 0% inhibition-AVG RLU 100% inhibition))*100%. Assay performance was evaluated according to the reference compound Staurosporine, which was within 3-fold of published results and between different tests, with a Z′ value larger than 0.5, where applicable.

Table 6A shows the activity of selected compounds of this invention in the assays described above. The compound numbers correspond to the compound numbers in Table 1.

TABLE 6A
Cell Viability IC₅₀'s in in U-87 MG and T98G Cell Lines

| Compound | U-87 MG | T98G IC₅₀ |
|---|---|---|
| No. | IC₅₀ (uM) | (uM) |
| Ibrutinib | >30 | >30 |
| I-16 | 1.3 | 2.2 |
| I-18 | 1.2 | 2.4 |

### Example 23: Cell-Based Cancer Efficacy Assays

### Results of Assays

Table 6B shows the activity of selected compounds of this invention in the assays described above. The compound numbers correspond to the compound numbers in Table 1.

TABLE 6B
Cell Viability IC₅₀'s in Ramos Cell Lines

| | GBM-U87MG | | GBM-T-98G | | GBM-U251MG | |
|---|---|---|---|---|---|---|
| | Cell | | Cell | | Cell | |
| | Proliferation | | Proliferation | | Proliferation | |
| Compound | IC₅₀ | Eₘₐₓ | IC₅₀ | Eₘₐₓ | IC₅₀ | Eₘₐₓ |
| # | uM | % | uM | % | uM | % |
| Ibrutinib | 28.5 | 52 | 24.4 | 73 | 0.31 | 57 |
| I-19 | 6.47 | 19 | >30 | 16 | 1.67 | 6 |
| I-19 + | 4.65 | 59 | >30 | 62 | 6.95 | 55 |
| Ibrutinib | | | | | | |
| I-11 | 0.66 | 96 | 3.28 | 98 | 0.93 | 82 |
| I-10 | 0.76 | 101 | 2.66 | 93 | 2.12 | 98 |

The dual inhibitor compounds of the invention gave surprising and unexpected activity, a 10-fold improvement compared to simply combining the two single activity agents (a CXCR4 inhibitor and a BTK inhibitor) and a 40-fold improvement compared to a selective BTK inhibitor.

### Example 24: Waldenstrom's Macroglobulinemia Assays

Waldenström's Macroglobulinemia cells (MWCL-1) and the bone marrow stromal cells (BMSCs; HS-27A) were used in this study. The MWCL-1 is a lymphoplasmacytic lymphoma established from the bone marrow aspirate of a 73-year-old male patient diagnosed with Waldenström's Macroglobulinemia. It was provided by Dr Stephen M. Ansell (MAYO file number 2021-121; 200 First Street SW, Rochester, Minnesota). This cell line was cultured in RPMI-1640 (Fisher Scientific) containing 10% fetal bovine serum (FBS) (Sigma Aldrich, St Louis, MO), 100U/ml of Penicillin-Streptomycin (Gibco™, ThermoFisher Scientific). The HS-27A bone marrow stromal cells were obtained from ATCC. They were maintained in RPMI 1640 (Fisher Scientific) supplemented with 100U/ml of Penicillin-Streptomycin (Gibco™, ThermoFisher Scientific) and 10% FBS (TakaraBio). Cells were cultured in a 37° C. tissue culture CO₂ incubator.

### Co-Culture System

For studies with compounds using a co-culture system of MWCL-1 and BMSCs, BMSCs were cultured in a 96-, 48- or 24-well plates and cultured until the 90% confluence was reached. Then MWCL-1 cells (density˜ 2×10⁵ cells/ml) were pretreated with various concentrations of dual antagonists (20-10-5-2.5-1.25 0.625-0.31 and 0.16 μM) or similar concentrations of the BTK inhibitors (Ibrutinib, Zanubrutinib, LOXO-305, ARQ-531) in a medium containing 4% FBS. After 1 hour, the MWCL-Icells were added to the BMSC to create a co-culture system. The cells were co-incubated for 72 hours. After incubation time, cell viability, apoptosis, and IgM release were measured.

### Cell Viability Assay

For studying the effect of compounds on the viability of Waldenström Macroglobulinemia cells (MWCL-1). Cellular viability was determined using the Cell Titer Glo Assay (Promega), which assesses metabolic activity as a proxy for viability, according to the manufacturer's instructions. MWCL-1 cells alone and the co-culture were seeded in the 96 well plates. Control and treated cells were run in duplicate and incubated. After 72 hours the plates were equilibrated at room temperature for 30 minutes, and 50 μL of CellTiter-Glo reagent was added to each well. The plates were shaken for 2 minutes and then incubated at room temperature for 10 minutes. The luminescence was measured with a microplate reader (Synergy™ HT, BioTek Instruments).

### Apoptosis Assay

For studying the effect of compounds on inducing apoptosis of Waldenström Macroglobulinemia cells (MWCL-1). Cells were washed with PBS, resuspended in Annexin V binding buffer (Biolegend), and stained with Alexa Fluor 647 annexin V (Biolegend), Propidium Iodide (BD Pharmigen), and CD45 antibody (Biolegend) for 15 minutes at RT and analyzed by flow cytometry. Data were collected on an CytoFLEX S Flow Cytometer (Beckman Coulter) and analysed with FCS Express Software (De Novo Software). Annexin V positive cells were considered apoptotic.

### Enzyme-Linked Immunosorbent Assay

For the IgM enzyme-linked immunosorbent assays (ELISAs), the human IgM ELISA Kit (Abcam) was used according to the manufacturer's recommendations. The supernatant of the MWCL-1 cells alone, HS-27A cells alone or of the co-culture was collected and plated into wells precoated with the monoclonal antibody specific for IgM. An IgM specific biotinylated detection antibody was added followed by washing with wash buffer. Subsequently, Streptavidin-Peroxidase Conjugate was added, and unbound conjugates were washed away with wash buffer. TMB was then used to visualize Streptavidin-Peroxidase enzymatic reaction. The Stop Solution was added and the microwell absorbance was read at 450 nm. Standards were tested together with the samples to obtain a standard curve.

As shown in the table below, compounds I-10 and I-11 were more effective in promoting apoptosis in WM cells (MWCL-1) than either ibrutinib or zanubrutinib alone. In the table, ** indicates an IC₅₀ of between 0.1 μM and 2.0 μM.

TABLE 7
Dual Antagonists More Effectively Increase Apoptosis in
WM Cells vs. BTK Inhibitors Alone

| | Apoptosis IC50 (μM) in single- |
|---|---|
| | mutant WM Cell Lines |
| Compound | (MWCL-1) |
| I-10 | ** |
| I-11 | ** |
| Ibrutinib | 4.2 |
| Zanubrutinib | 17.7 |

### Example 25: Kinase Screening

The compound I-10, a dual inhibitor according to the present invention, and four leading clinical BTK inhibitors (Zanubrutinib, LOXO-305, Ibrutinib and ARQ-531) were screened against a panel of approximately 336 wild-type kinases [PanQinase™, Reaction Biology Europe GmbH, Freiburg Germany]. Screening procedures were performed in accordance with the company's on-line protocol [accessed on-line Mar. 25, 2022 at reaction biology.com/assay-protocol-panqinase]. The results for 70 wild-type kinases of interest are shown in the table below. Inhibitory activity of 50% or greater at concentrations of 1 μM are highlighted for all five compounds tested.

In comparison with the other compounds tested, the kinase selectivity profile of I-10 demonstrated surprisingly high selectivity for BTK inhibition, with unexpectedly low off-target effects. Apart from BTK, I-10 showed 90% or greater inhibitor effect on only one other kinase tested, ACK1. Notably, ACK1 has been reported to be aberrantly activated, amplified or mutated in a wide variety of human cancers. Mahajan and Mahajan (2013) Cancer Letters 338:185-192. I-10 unexpectedly showed surprising and inhibitory effects [50-89% inhibition at 1 μM] on a cluster of 13 additional kinases: AuroraA; DDR2; TRKA; ABL1; ABL2; CDK13/CycK; CLK1; CLK2; LIMK1; LIMK2; MAP2K11; MUSK and SIK2. These kinases are associated with various types of cancers. Some are targeted by clinical BCR-ABL kinase inhibitors, such as imatinib (Gleevec®, Novartis), bosutinib (Bosulif®, Pfizer), dasatinib (Sprycel®, Bristol-Myers Squbb), nilotinib (Tasigna®, Novartis) and ponatinib (Iclusig®, Takeda), which are indicated for treatment of Philadelphia chromosome positive chronic myeloid leukemia (CML) and/or acute lymphoblastic leukemia (ALL). While ARQ-531 exhibited inhibitory effects of 50% or greater on three of these kinases [AuroraA; DDR2 and TRKA] none of the other BTK inhibitors tested exhibited inhibitory effects of 50% or more on these 13 kinases.

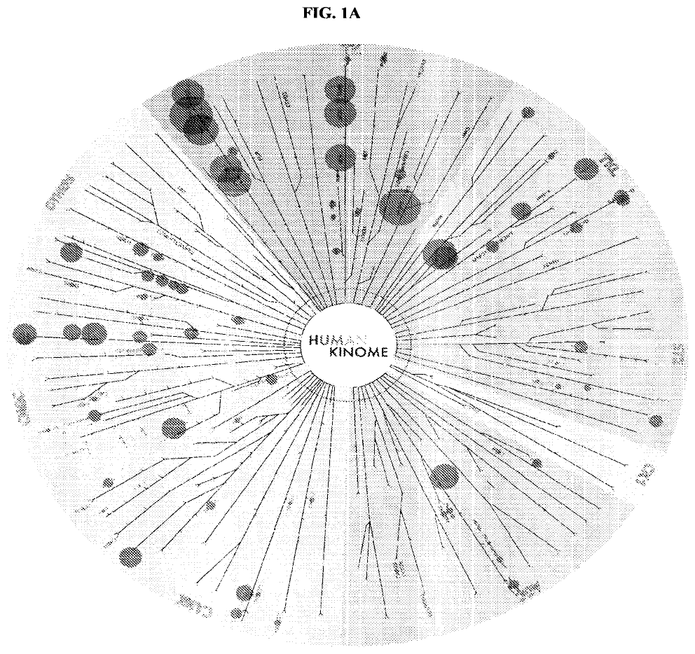

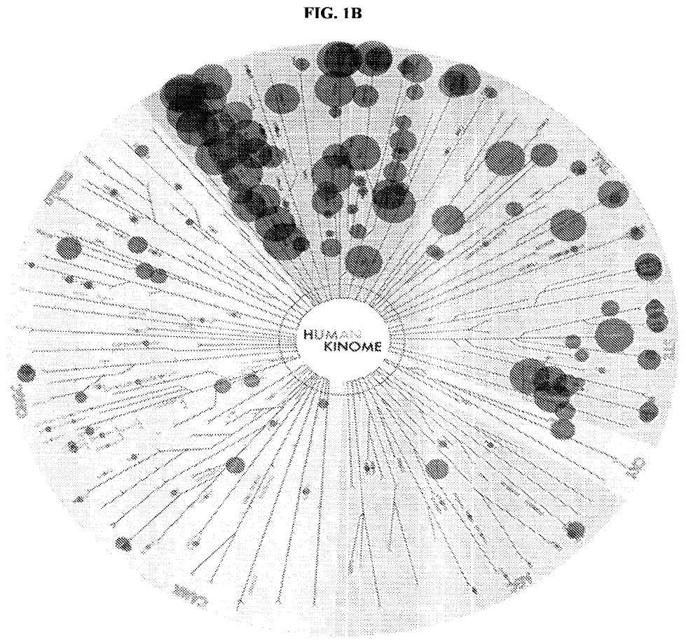

FIG. 1A provides a visualization of the data of the table of kinase inhibition below for compound I-10. FIG. 1B shows similar visualization for kinase inhibition by ARQ-531. The visualizations were performed using a web-based kinase mapper tool provided on the developer's website accessed on-line Mar. 25, 2022 at reactionbiology.com/tools/kinase-mapper. The diameter of dots reflects the percent inhibition of 336 tested wild-type kinases. In comparison with the visualization for ARQ-531, the inhibitory effects of I-10 are shown to be surprisingly and unexpectedly precise and directed. The selectivity of I-10 and other compounds of the invention is expected to lead to reduced adverse effects in patients due to off-target binding.

| Kinase Name | Zanubrutinib | LOXO-305 | Ibrutinib | ARQ-531 | I-10 |
|---|---|---|---|---|---|
| BMX | ++++ | ++ | ++++ | ++++ | +++ |
| BTK | ++++ | ++++ | ++++ | ++++ | ++++ |
| TEC | ++++ | ++++ | ++++ | ++++ | +++ |
| BRK | ++++ | +++ | ++++ | ++++ | + |
| CSK | ++ | +++ | ++++ | ++++ | + |
| ERBB4 | +++ | ++ | ++++ | ++++ | + |
| TXK | ++++ | ++ | ++++ | ++++ | + |
| ACK1 | + | + | +++ | ++++ | ++++ |
| PKN3 | + | + | +++ | ++ | ++ |
| BLK | +++ | + | ++++ | ++++ | + |
| ERBB2 | + | ++ | ++++ | +++ | + |
| FRK | ++ | + | +++ | +++ | + |
| RET | + | ++ | ++ | ++++ | + |
| RIPK2 | ++ | + | +++ | ++++ | + |
| ARAF YDYD | + | + | ++ | +++ | + |
| EGFR | + | + | +++ | +++ | + |
| EPHB2 | + | + | ++ | +++ | + |
| FGFR1 | + | + | ++ | ++ | + |
| FGFR2 | + | + | | ++++ | + |
| FGFR3 | + | + | ++ | ++ | + |
| FGR | + | + | +++ | +++ | + |
| FYN | + | + | +++ | ++++ | + |
| HCK | + | + | +++ | +++ | + |
| LCK | + | + | ++++ | ++++ | + |
| LYN | + | + | +++ | +++ | + |
| RAF1 YDYD | + | + | ++ | +++ | + |
| RIPK5 | + | + | ++ | +++ | + |
| SRC | + | + | ++ | +++ | + |
| SRMS | + | + | ++++ | ++++ | + |
| VEGFR2 | + | + | ++ | | + |
| YES | + | + | +++ | ++++ | + |
| AuroraA | + | + | + | ++ | ++ |
| DDR2 | + | + | + | ++ | +++ |
| TRKA | + | + | + | ++++ | +++ |
| ABL1 | + | + | + | | +++ |
| ABL2 | + | + | + | | +++ |
| CDK13/CycK | + | + | + | | ++ |
| CLK1 | + | + | + | | ++ |
| CLK2 | + | + | + | | ++ |

| Kinase Name | Zanubrutinib | LOXO-305 | Ibrutinib | ARQ-531 | I-10 |
|---|---|---|---|---|---|
| LIMK1 | + | + | + | | +++ |
| LIMK2 | + | + | + | | ++ |
| MAP3K11 | + | + | + | | ++ |
| MUSK | + | + | + | | +++ |
| SIK2 | + | + | + | | ++ |
| CK1epsilon | + | + | + | ++ | + |
| EPHA6 | + | + | + | ++ | + |
| FER | + | + | + | +++ | + |
| FES | + | + | + | ++ | + |
| INSR | + | + | + | +++ | + |
| INSRR | + | + | + | ++++ | + |
| ITK | + | + | + | ++ | + |
| JAK3 | + | + | + | ++ | + |
| KIT | + | + | + | ++++ | + |
| LRRK2 | + | + | + | ++ | + |
| MAP4K5 | + | + | + | ++ | + |
| MEK1 | + | + | + | ++ | + |
| MEKK2 | + | + | + | +++ | + |
| MEKK3 | + | + | + | ++ | + |
| MINK1 | + | + | + | ++ | + |
| MKK6 SDTD | + | + | + | ++++ | + |
| MST1 | + | + | + | +++ | + |
| p38beta | + | + | + | +++ | + |
| PAK1 | + | + | + | ++ | + |
| PAK2 | + | + | + | +++ | + |
| PAK3 | + | + | + | ++++ | + |
| PDGFRalpha | + | + | + | +++ | + |
| PDGFRbeta | + | + | +++ | + | + |
| TIE2 | + | + | ++ | + | + |
| TRKB | + | ++ | + | + | + |
| TRKC | + | ++ | + | + | + |

| CODE | INHIBITORY EFFECT @ 1 uM |
|---|---|
| ++++ | 90%-100% Inhibition at 1 uM |
| +++ | 70%-89% Inhibition at 1 uM |
| ++ | 50%-69% Inhibition at 1 uM |
| + | Below 50% Inhibition at 1 uM |

For each kinase, the median value of the cpm of three wells was defined as “low control” (n=3). This value reflects unspecific binding of radioactivity to the plate in the absence of a protein kinase but in the presence of the substrate. Additionally, for each kinase the median value of the cpm of three other wells was taken as the “high control,” i.e. full activity in the absence of any inhibitor (n=3). The difference between high and low control of each enzyme was taken as 100% activity.

As part of the data evaluation the low control of each kinase was subtracted from the high control value as well as from their corresponding “compound values”. The residual activity (in %) for each compound well was calculated by using the following formula: Res. Activity (%)=100×[(signal of compound−low control)/(high control−low control)].

The selectivity score, according to Karaman et al. (Nat. Biotech. 26, 1, 127-132 (2008)), is a compound concentration-dependent parameter describing the portion of kinases, which are inhibited to more than a predefined degree (e.g., more than 50%), in relation to all tested kinases of the particular project.

The selectivity score of the compounds at the tested concentrations was calculated for a residual activity <50%, i.e., an inhibition of >50%. The selectivity score for a particular compound at a particular concentration was calculated by using the following formula: Selectivity Score=(count of data points <50%)/(total number of data points).

The selectivity scores (<50% residual kinase activity) for additional compounds can be found in the table below.

Table 8: (Mean) selectivity scores for compounds at 1 μM concentrations in 340 wild-type protein kinase assays; *: no residual activity <50%.

| Compound ID | Zanubrutinib | Ibrutinib | BIIB-068 |
|---|---|---|---|
| Selectivity Score | 0.029 | 0.097 | 0.009 |

| | | LOXO- | |
|---|---|---|---|
| Compound ID | ARQ-531 | 305-P1 | I-11 |
| Selectivity Score | 0.165 | 0.032 | 0.032 |

| Compound ID | I-16 | I-18 | I-10 |
|---|---|---|---|
| Selectivity Score | 0.265 | 0.221 | 0.053 |

### Example 26: BTK Screening Assay (33PanQinase® Activity Assay)

Inhibition of BTK activity was evaluated using a radiometric protein kinase assay (33PanQinase® Activity Assay) to measure the activity of BTK (Bruton tyrosine kinase). IC₅₀ values were measured by testing 10 concentrations of test compound in singlicate, with a serial semi-logarithmic dilution from a top concentration of 10 μM (final DMSO concentration 1%).

Kinase assays were performed in 96-well ScintiPlates™ from Perkin Elmer (Boston, MA, USA) in a 50 μL reaction volume. The reaction cocktail was pipetted in four steps in the following order: 10 μL of non-radioactive ATP solution (in H₂O); 25 μL of assay buffer/[γ-³³P]-ATP mixture; 5 μL of compound test sample in 10% DMSO; 10 μL of enzyme/substrate mixture.

The assay for the protein kinase contained 70 mM HEPES-NaOH pH 7.5, 3 mM MgCl₂, 3 mM MnCl₂, 3 μM Na-orthovanadate, 1.2 mM DTT, 50 μg/ml PEG20000, 3 μM ATP, [γ-33P]-ATP (approx. 4×1005 cpm per well), 150 ng/50 μL BTK kinase (Reaction Biology Europe GmbH, Lot 012,11), and 250 ng/50 μL Poly(Glu,Tyr)4:1 substrate (Lot SIG_20K5903). BTK kinase was expressed in Sf9 insect cells or in E. coli as recombinant GST-fusion proteins or His-tagged proteins, either as full-length or enzymatically active fragments from human cDNAs, and purified by either GSH-affinity chromatography or immobilized metal. Purity was examined by SDS-PAGE/Coomassie staining, and identity confirmed by mass spectroscopy.

The reaction cocktails were incubated at 30° C. for 60 minutes. The reaction was stopped with 50 μL of 2% (v/v) H₃PO₄, plates were aspirated and washed two times with 200 μL 0.9% (w/v) NaCl. Incorporation of ³³Pᵢ (counting of “cpm”) was determined with a microplate scintillation counter (Microbeta, Wallac).

The median value of the counts in wells containing assay buffer without enzyme, and without test compound was defined as “low control.” This value reflects unspecific binding of radioactivity to the plate in the absence of a protein kinase but in the presence of the substrate. The median value of the counts in wells containing assay buffer without test compound was defined as the “high control,” i.e. full activity in the absence of any inhibitor. The difference between high and low control was taken as 100% activity. Residual activity (in %) was calculated using the following formula: Res. Activity (%)=100×[(cpm of compound-low control)/(high control-low control)].

The residual activities for each concentration and the compound IC₅₀ values were calculated using Quattro Workflow V3.1.1 (Quattro Research GmbH, Munich, Germany; quattro-research.com). The fitting model for the IC₅₀ determinations was “Sigmoidal response (variable slope)” with parameters “top” fixed at 100% and “bottom” at 0%. The fitting method used was a least-squares fit.

Table 9 shows the activity of selected compounds of this invention in the assays described above. The compound numbers correspond to the compound numbers in Table 1. Compounds having an activity designated as “A” provided an IC₅₀ of 1 to 100 nM; compounds having an activity designated as “B” provided an IC₅₀ of >100 nm to <1 μM; compounds having an activity designated as “C” provided an IC₅₀ of 1 μM to <2.5 μM; and compounds having an activity designated as “D” provided an IC₅₀ of 2.5 μM or greater.

TABLE 9
Inhibition of BTK (33PanQinase ® Activity Assay)

| | 33PanQinase ® |
|---|---|
| Compound No. | Radiometric IC50 |
| I-1 | B |
| I-10 | A |
| I-11 | A |
| I-12 | A |
| I-13 | B |
| I-14 | A |
| I-15 | C |
| I-16 | A |
| I-17 | A |
| I-18 | A |
| I-2 | C |
| I-20 | A |
| I-21 | A |
| I-22 | A |
| I-26 | C |
| I-27 | B |
| I-28 | C |
| I-29 | A |
| I-3 | A |
| I-30 | B |
| I-31 | B |
| I-32 | A |
| I-33 | B |
| I-34 | B |
| I-35 | A |
| I-36 | C |
| I-37 | B |
| I-38 | A |
| I-39 | B |
| I-4 | A |
| I-40 | A |
| I-41 | A |
| I-42 | A |
| I-43 | A |
| I-44 | A |
| I-45 | A |
| I-46 | A |
| I-47 | A |
| I-48 | A |
| I-49 | A |
| I-5 | B |
| I-50 | A |
| I-51 | A |
| I-52 | A |
| I-53 | A |
| I-54 | A |
| I-55 | A |
| I-56 | A |
| I-57 | A |
| I-58 | A |
| I-59 | A |
| I-6 | A |
| I-60 | A |
| I-61 | D |
| I-63 | D |
| I-64 | D |
| I-66 | A |
| I-67 | A |
| I-68 | A |
| I-69 | A |
| I-7 | A |
| I-70 | B |
| I-71 | C |
| I-73 | B |
| I-74 | D |
| I-75 | D |
| I-76 | D |
| I-77 | D |
| I-78 | D |
| I-79 | D |
| I-8 | A |
| I-80 | B |
| I-81 | B |
| I-82 | B |
| I-83 | B |
| I-84 | B |
| I-85 | B |
| I-86 | B |
| I-87 | D |
| I-88 | D |
| I-89 | D |
| I-9 | B |
| I-90 | D |
| I-91 | D |
| I-92 | C |

### REFERENCES

1. Buske C, Leblond V, Dimopoulos M, et al. Waldenstrom's macroglobulinaemia: ESMO Clinical Practice Guidelines for diagnosis, treatment and follow-up. Ann Oncol. 2013; 24(6):vi155-9.
2. Cao Y, Hunter Z, Liu X, et al. The WHIM-like CXCR4 (S338X) somatic mutation activates AKT and ERK, and promotes resistance to ibrutinib and other agents used in the treatment of Waldenstrom's Macroglobulinemia. Leukemia. 2014; 29(1):169-76.
3. Cao Y, Hunter Z, Liu X, et al. CXCR4 WHIM-like frameshift and nonsense mutations promote ibrutinib resistance but do not supplant MYD88 (L265P)-directed survival signaling in Waldenstrom macroglobulinaemia cells. Br J Haematol. 2015; 168(5):701-7.
4. Castillo J, Olszewski A, Kanan S, et al. Overall survival and competing risks of death in patients with Waldenstrom macroglobulinaemia: an analysis of the Surveillance, Epidemiology and End Results database. Br J Haematol. 2015; 169(1):81-9.
5. Chatterjee S, Azad B, Nimmagadda S. The intricate role of CXCR4 in cancer. Adv Cancer Res. 2014; 124:31-82.
6. Christiansen C F, Johansen M B, Langeberg W J, et al. Incidence of acute kidney injury in cancer patients: a Danish population-based cohort study. Eur J Intern Med. 2011; 4:399-406.
7. Dale D, Bolyard A, Kelley M, et al. The CXCR4 antagonist plerixafor is a potential therapy for myelokathexis, WHIM syndrome. Blood. 2011; 118(18):4963-66. doi: 10.11.1182/blood-2011-06-360586
8. de Duve C, de Barsy T, Poole B, et al. Lysosomotropic agents. Biochem Pharmacol. 1974; 23:2495-2534.
9. De Falco V, Guarino V, Avilla E, et al. Biological role and potential therapeutic targeting of the chemokine receptor CXCR4 in undifferentiated thyroid cancer. Cancer Res 2007; 67:11821-9.
10. Dimopoulos M, Panayiotidis P, Moulopoulos L, et al. Waldenstrom's macroglobulinemia: clinical features, complications, and management. J Clin Oncol. 2000; 18(1):214-26.
11. Dimopoulos M, Anagnostopoulos A, Kyrtsonis M, et al. Primary treatment of Waldenstrom macroglobulinemia with dexamethasone, rituximab, and cyclophosphamide. J Clin Oncol. 2007; 25(22):3344-9.
12. Dimopoulos M, Kastritis E, Owen R, et al. Treatment recommendations for patients with Waldenstrom macroglobulinemia (WM) and related disorders: IWWM-7 consensus. Blood. 2014; 124(9):1404-11.
13. Dimopoulos M, Trotman J, Tedeschi A, et al. Ibrutinib for patients with rituximab-refractory Waldenstrom's macroglobulinaemia (INNOVATE): an open-label substudy of an international, multicentre, Phase 3 trial. Lancet Oncol. 2017; 18(2):241-250.
14. Dimopoulos M, Tedeschi A, Trotman J, et al. Phase 3 Trial of Ibrutinib plus Rituximab in Waldenstrom's Macroglobulinemia. N Engl J Med. 2018; 378(25):2399-2410.
15. FDA Drug development and drug interactions: table of substrates, inhibitors and inducers. Updated Nov. 14, 2017. Available at fda.gov/drugs/drug-interactions-labeling/drug-development-and-drug-interactions-table-substrates-inhibitors-and-inducers. Accessed Jun. 22, 2019.
16. Fonseca R, Hayman S. Waldenstrom macroglobulinaemia. Br J Haematol. 2007; 138(6):700-20.
17. Goldman J, Mendenhall M, Rettinger S. Hyperglycemia associated with targeted oncologic treatment: mechanisms and management. The Oncologist. 2016; 21:1326-36.
18. Groves F, Travis L, Devesa S, et al. Waldenstrom's macroglobulinemia: incidence patterns in the United States, 1988-1994. Cancer. 1998; 82(6):1078-81.
19. Hassan S, Buchanan M, Jahan K, et al. CXCR4 peptide antagonist inhibits primary breast tumor growth, metastasis and enhances the efficacy of anti-VEGF treatment or docetaxel in a transgenic mouse model. Int J Cancer 2010; 129:225-32.
20. Hendrix C, Flexner C, MacFarland R, et al. Pharmacokinetics and safety of AMD-3100, a novel antagonist of the CXCR-4 cytokine receptor, in human volunteers. Antimicrob Agent and Chemother. 2000; 44(6):1167-73.
21. Hruban Z. Pulmonary changes induced by amphophilic drugs. Environ Health Perspect. 1976; 16:111-18
22. Huang E H, Singh B, Cristofanilli M, et al. A CXCR4 antagonist CTCE-9908 inhibits primary tumor growth and metastasis of breast cancer. J Surg Res 2009; 155:231-6.
23. Hunter Z, Xu L, Yang G, et al. The genomic landscape of Waldenstrom macroglobulinemia is characterized by highly recurring MYD88 and WHIM-like CXCR4 mutations, and small somatic deletions associated with B-cell lymphomagenesis. Blood. 2014; 123(11):1637-46.
24. IMBRUVICA® [Highlights of Prescribing Information]. Horsham, P A: Janssen Biotech, Inc; 2019. imbruvica.com/docs/librariesprovider7/default-document-library/prescribing-information.pdf
25. Ji Y, Jin J, Hyman D, et al. Challenges and opportunities in dose finding in oncology and immuno-oncology. Clin Transl Sci. 2018; 11:345-51.
26. Jiang H, Passarelli P, Munro G, et al. High-resolution sub-cellular imaging by correlative NanoSIMS and electron microscopy of amiodorone internalisation by lung macrophages as evidence for drug-induced phospholipidosis. Chem Commun. 2017; 53:1506-1509.
27. Kim S Y, Lee C H, Midura B V, et al. Inhibition of the CXCR4/CXCL12 chemokine pathway reduces the development of murine pulmonary metastases. Clin Exp Metastasis 2008; 25:201-11.
28. Kodovanti U, Mehendale H. Cationic amphiphilic drugs and phospholipid storage disorder. Pharmcol Review. 1990; 42(4):327-54.
29. Kyle R A, Therneau™, Rajkumar S V, et at. Long-term follow-up of 241 patients with monoclonal gammopathy of undetermined significance: the original Mayo Clinic series 25 years later. Mayo Clin Proc. 2004; 79:859-65.
30. Kyle R A, Rajkumar S V. Monoclonal gammopathy of undetermined significance. Clin Lymphoma Myeloma. 2005; 6:102-14.
31. Lahoti A, Humphreys B. Chapter 3 AKI associated with malignancies. Amer Soc Nephrol. 2016; 3:1-8.
32. Lei N, Rubin E, Mehrotra N, et al. Rendering the 3±3 design to rest: more efficient approaches to oncology dose-finding trials in the era of targeted therapy. Clin Cancer Resch. 2016; 22(11):2623-9.
33. Liu Q, Li Z, Gao J L, et al. CXCR4 antagonist AMD3 100 redistributes leukocytes from primary immune organs to secondary immune organs, lung, and blood in mice. Eur J Immunol. 2015:45 (6):1855-67.
34. Liu Q, Pan C, Lopez L, et al. WHIM syndrome caused by Waldenstrom's macroglobulinemia-associated mutation CXCR4 (L329fs). J Clin Immunol. 2016; 36(4):397-405.
35. Majumdar S, Murphy P M. Adaptive immunodeficiency in WHIM syndrome. Int J Mol Sci. 2018; 20(1):3. Published 2018. doi: 103390/ijms20010003
36. Maker A V, Yang, J C, Sherry R M, et al. Intrapatient dose escalation of anti-CTLA-4 antibody in patients with metastatic melanoma. J Immunother. 2006; 29 (4): 455-463.
37. Mazzucchelli M, Frustaci A, Deodato M, et al. Waldenstrom's macroglobulinemia: an update. Mediterr J Hematol Infect Dis. 2018; 10(1) e2018004. doi: 10.4084/MJHID.2018.004
38. Moyle G, DeJesus E, Boffito M, et al. Proof of activity with AMD11070, an orally bioavailable inhibitor of CXCR4 tropic HIV type 1. Clin Infect Dis. 2009; 48(6):798-805.
39. Muller A, Homey B, Soto H, et al. Involvement of chemokine receptors in breast cancer metastasis. Nature 2001; 410:50-6.
40. Murugan R, Karajala-Subramanyam V, Lee M, et al. Acute Kidney injury in non-severe pneumonia is associated with an increased immune response and lower survival. Kidney Int. 2010; 77(6):527-35. doi: 10.1038/ki.2009.502
41. Nagase H, Miyamasu M, Yamaguchi M, et al. Expression of CXCR4 in eosinophils: functional analyses and cytokine-mediated regulation. J Immunol. 2000; 164(11):5935-43.
42. Nowakowski G S, Witzig T E, Dinghi D, et al. Circulating plasma cells detected by flow cytometry as a predictor of survival in 302 patients with newly diagnosed multiple myeloma. Blood. 2005; 106:2276-79.
43. Nyunt M, Becker S, MacFarland R, et al. Pharmacokinetic Effect of AMD070, an Oral CXCR4 Antagonist, on CYP3A4 and CYP2D6 Substrates Midazolam and Dextromethorphan in Healthy Volunteers. J Acquir Immune Defic Syndr. 2008; 47 (5): 559-565.
44. Olszewski A, Treon S, Castillo J. Evolution of Management and Outcomes in Waldenstrom Macroglobulinemia: A Population-Based Analysis. Oncologist. 2016; 21(11):1377-1386.
45. Owen R, Treon S, Al-Katib A, et al. Clinicopathological definition of Waldenstrom's macroglobulinemia: consensus panel recommendations from the Second International Workshop on Waldenstrom's Macroglobulinemia. Semin Oncol. 2003; 30(2):110-5.
46. Owen R, Pratt G, Auer R, et al. Guidelines on the diagnosis and management of Waldenstrom macroglobulinaemia. Br J Haematol. 2014; 165(3):316-33.
47. Porvasnik S, Sakamoto N, Kusmartsev S, et al. Effects of CXCR4 antagonist CTCE-9908 on prostate tumor growth. Prostate 2009; 69:1460-9.
48. Pophali P, Bartley A, Kapoor P, et al. Prevalence and survival of smoldering Waldenstrom macroglobulinaemia in the United States. Br J Haematol. 2018. 1011-70.
49. Poulain S, Roumier C, Venet-Caillault A, et al. Genomic Landscape of CXCR4 Mutations in Waldenstrom Macroglobulinemia. Clin Cancer Res. 2016; 22(6):1480-8.
50. Roccaro A, Sacco A, Himenez C, et al. C1013G/CXCR4 acts as a driver mutation of tumor progression and modulator of drug resistance in lymphoplasmacytic lymphoma. Blood. 2014; 123 (26): 4120-31.
51. Rubin J B, Kung A L, Klein R S, et al. A small-molecule antagonist of CXCR4 inhibits intracranial growth of primary brain tumors. Proc Nat Acad Sci USA 2003; 100:13513-8.
52. Sacco A, Fenotti A, Affo, L, et al. The importance of the genomic landscape in Waldenstrom's macroglobulinemia of targeted therapeutical intervention. Oncotarget. 2017; 8:35435-44.
53. Sarma J S M, Pei H, Venkataraman K. Role of oxidative stress in amiodorone-induced toxicity. J Cardiovasc Pharmacol Therapeut. 1997; 2(1):53-60.
54. Schimanski C C, Bahre R, Gockel I, et al. Dissemination of hepatocellular carcinoma is mediated via chemokine receptor CXCR4. Br J Cancer 2006; 95:210-7.
55. Smith M C, Luker K E, Garbow J R, et al. CXCR4 regulates growth of both primary and metastatic breast cancer. Cancer Res 2004; 64:8604-12.
56. Stone N, Dunaway S, Flexner C, et al. Multiple-dose escalation study of the safety, pharmacokinetics and biologic activity of oral AMD070, a selective CXCR4 receptor inhibitor, in human subjects. Antimicrob Agent and Chemother. 2007; 51(7):2351-58/-73.
57. Treon S, XIII. Waldenstrom's macroglobulinaemia: an indolent B-cell lymphoma with distinct molecular and clinical features. Hematol Oncol. 2013; 31(1):76-80.
58. Treon S, Cao Y, Xu L, et al. Somatic mutations in MYD88 and CXCR4 are determinants of clinical presentation and overall survival in Waldenstrom macroglobulinemia. Blood. 2014; 123(18):2791-6.
59. Treon S, Tripsas C, Meid K, et al. Ibrutinib in previously treated Waldenstrom's macroglobulinemia. N Engl J Med. 2015; 372(15):1430-40. [1]
60. Treon S, How I treat Waldenstrom macroglobulinemia. Blood. 2015; 126(6):721-32. [2]
61. Treon S, Gustine J, Meid K, et al. Ibrutinib monotherapy in symptomatic, treatment-naive patients with Waldenstrom macroglobulinemia. J Clin Oncol. 2018; 36(27):2755-2761. [1]
62. Treon S, Meid K, Gustine J, et al. Ibrutinib shows prolonged progression-free survival in symptomatic, previously treated patients with MYD88 mutated Waldenstrom's macroglobulinemia: long-term follow-up of pivotal trial (NCT01614821). Abstr. PS1185. 23rd Conf. Euro Hem Assoc. Jun. 16, 2018. Stockholm, Sweden. [2]
63. Varettoni M, Zibellini S, Defrancesco I, et al. Pattern of somatic mutations in patients with Waldenström macroglobulinemia or IgM monoclonal gammopathy of undetermined significance. Haematologica. 2017; 102(12):2077-2085.
64. Xu L, Hunter Z, Tsakmaklis N, et al. Clonal architecture of CXCR4 WHIM-like mutations in Waldenstrom Macroglobulinaemia. Br J Haematol. 2016; 172(5):735-44.
65. Zeelenberg I S, Ruuls-Van Stalle L E, Roos E. The chemokine receptor CXCR4 is required for outgrowth of colon carcinoma micrometastases. Cancer Res 2003; 63:3833-9.
66. Beider K, Ribakovsky E, Abraham M, Wald H, Weiss L, Rosenberg E, Galun E, Avigdor A, Eizenberg O, Peled A, Nagler A. Targeting the CD20 and CXCR4 pathways in non-hodgkin lymphoma with rituximab and high-affinity CXCR4 antagonist BKT140. Clin Cancer Res. 2013 Jul. 1; 19(13):3495-507. doi: 10.1158/1078-0432.CCR-12-3015. Epub 2013 May 1. PMID: 23637121.
67. Chu Y, Lee S, Shah T, Yin C, Barth M, Miles R R, Ayello J, Morris E, Harrison L, Van de Ven C, Galardy P, Goldman S C, Lim M S, Hermiston M, McAllister-Lucas L M, Giulino-Roth L, Perkins S L, Cairo M S. Ibrutinib significantly inhibited Bruton's tyrosine kinase (BTK) phosphorylation, in-vitro proliferation and enhanced overall survival in a preclinical Burkitt lymphoma (BL) model. Oncoimmunology. 2018 Oct. 11; 8(1): e1512455. doi: 10.1080/2162402X.2018.1512455. PMID: 30546948; PMCID: PMC6287791.
68. Zhou Y, Larsen P H, Hao C, Yong V W. CXCR4 is a major chemokine receptor on glioma cells and mediates their survival. J Biol Chem. 2002 Dec. 20; 277(51):49481-7. doi: 10.1074/jbc.M206222200. Epub 2002 Oct. 17. PMID: 12388552.
69. Yue C, Niu M, Shan Q Q, Zhou T, Tu Y, Xie P, Hua L, Yu R, Liu X. High expression of Bruton's tyrosine kinase (BTK) is required for EGFR-induced NF-κB activation and predicts poor prognosis in human glioma. J Exp Clin Cancer Res. 2017 Sep. 25; 36(1):132. doi: 10.1186/s13046-017-0600-7. PMID: 28946903; PMCID: PMC5613332.

1. A compound of formula I: 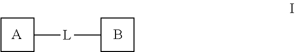
or a pharmaceutically acceptable salt thereof, wherein: [symbol]
is a small molecule BTK inhibitor; [symbol]
is a small molecule or peptide CXCR4 inhibitor, or a CXCR4 antibody; and
-L- is a covalent bond or a bivalent linker;
wherein -L- is attached via a covalent bond to each of [symbol] and [symbol] at a carbon, nitrogen, or oxygen atom, and each of the covalent bonds independently replaces a hydrogen on the carbon, nitrogen, or oxygen atom.

2. The compound of claim 1, wherein the compound is of formula II: 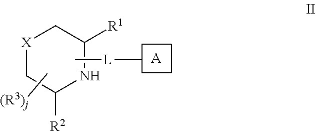
or a pharmaceutically acceptable salt, solvate, or hydrate thereof, wherein:
—X— is carbon, nitrogen, oxygen, sulfur, SO, or SO₂;
-L- is a covalent bond or a C₁₋₈ bivalent straight or branched, saturated or unsaturated hydrocarbon chain wherein 1-4 methylene units of the chain are independently and optionally replaced with —O—, —C(O)—, —C(S)—, -Cy-, —C(R^{A})₂—, —CH(R^{A})—, —CH(OR)—, —N(R)—, —S—, —S(O)—, or —S(O)₂—;
each R^{A} is independently hydrogen, halogen, —CN, optionally substituted group selected from C₁₋₆ aliphatic; phenyl; an 8-10 membered bicyclic aryl ring, a 3-7 membered saturated or partially unsaturated monocyclic carbocyclic ring; a 4-8 membered saturated or partially unsaturated monocyclic heterocyclic ring having 1-2 heteroatoms independently selected from nitrogen, oxygen, and sulfur; a 5-6 membered monocyclic heteroaryl ring having 1-4 heteroatoms independently selected from nitrogen, oxygen, and sulfur; and an 8-10 membered bicyclic heteroaryl ring having 1-5 heteroatoms independently selected from nitrogen, oxygen, and sulfur; or
two R^{A} groups on the same carbon are optionally taken together, with the carbon they are attached to, to form:
an optionally substituted 3-10 membered monocyclic or bicyclic saturated, or partially unsaturated, carbocyclic ring; or
an optionally substituted 3-10 membered monocyclic or bicyclic saturated, or partially unsaturated, heterocyclic ring, having 0-3 heteroatoms independently selected from nitrogen, oxygen, and sulfur;
each R is independently hydrogen, or an optionally substituted group selected from C₁₋₆ aliphatic; phenyl; an 8-10 membered bicyclic aryl ring, a 3-7 membered saturated or partially unsaturated monocyclic carbocyclic ring; a 4-8 membered saturated or partially unsaturated monocyclic heterocyclic ring having 1-2 heteroatoms independently selected from nitrogen, oxygen, and sulfur; a 5-6 membered monocyclic heteroaryl ring having 1-4 heteroatoms independently selected from nitrogen, oxygen, and sulfur; and an 8-10 membered bicyclic heteroaryl ring having 1-5 heteroatoms independently selected from nitrogen, oxygen, and sulfur; or
two R groups on the same nitrogen are optionally taken together with the nitrogen to form an optionally substituted 4-7 membered monocyclic saturated, partially unsaturated, or heteroaryl ring having, in addition to the nitrogen, 0-3 heteroatoms independently selected from nitrogen, oxygen, and sulfur;
each -Cy- is independently an optionally substituted bivalent ring selected from a 4-7 membered saturated or partially unsaturated heterocyclylenyl having 1-2 heteroatoms independently selected from nitrogen, oxygen, and sulfur, phenylenyl, a 3-7 membered saturated or partially unsaturated carbocyclylenyl, or a 5-6 membered heteroarylenyl having 1-4 heteroatoms independently selected from nitrogen, oxygen, and sulfur;
each of R¹ and R² is independently —C(O)N(R″)₂; —C(O) OR″; C₁₋₇ aliphatic; phenyl; a 5-6 membered monocyclic heteroaryl ring having 1-4 heteroatoms independently selected from nitrogen, oxygen, and sulfur; a 3-7 membered saturated or partially unsaturated monocyclic carbocyclic ring; a 4-8 membered saturated or partially unsaturated monocyclic heterocyclic ring having 1-2 heteroatoms independently selected from nitrogen, oxygen, and sulfur; a 5-10 membered saturated or partially unsaturated bridged bicyclic ring having 0-3 heteroatoms independently selected from nitrogen, oxygen, and sulfur; a 5-10 membered saturated or partially unsaturated spirocyclic ring having 0-3 heteroatoms independently selected from nitrogen, oxygen, and sulfur; or an 8-10 membered partially aromatic or heteroaromatic bicyclic heterocyclic ring having 1-4 heteroatoms independently selected from nitrogen, oxygen, and sulfur; each of which is substituted with m instances of R′;
each occurrence of R′ is independently hydrogen, halogen, or a C₁₋₆ aliphatic group optionally substituted with 1-5 halogens;
each occurrence of R″ is independently hydrogen, or a C₁₋₆ aliphatic group optionally substituted with 1-5 halogens;
each R³ is independently —N(R)₂, —OR, or C₁₋₇ aliphatic optionally substituted with 1-5 groups independently selected from halogen, —N(R)₂, and —OR, and wherein 1 or 2 methylene units of the aliphatic group are optionally replaced by —N(R)— or a methyl group is optionally replaced by —N(R)₂; [symbol]
is selected from the following: 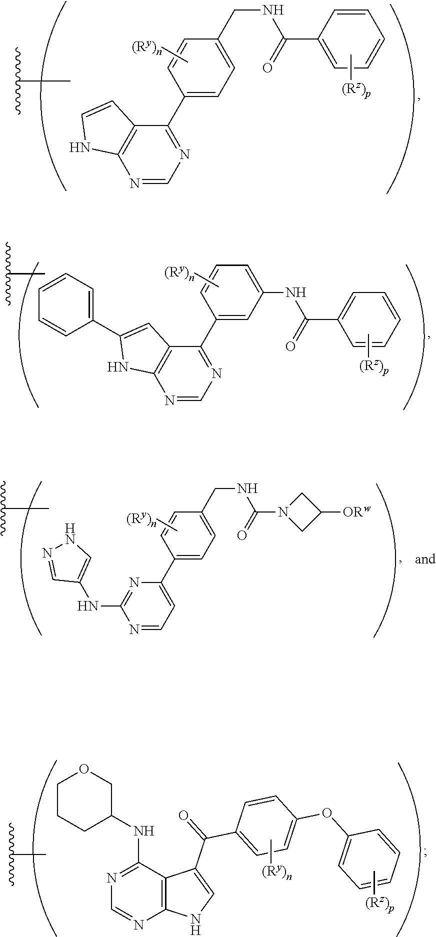
R^{w} is hydrogen, or C₁₋₇ aliphatic optionally substituted with 1-5 halogens;
R^{y} is hydrogen, halogen, or C₁₋₇ aliphatic optionally substituted with 1-5 halogens;
R^{z} is hydrogen, halogen, or C₁₋₇ aliphatic optionally substituted with 1-5 halogens;
j is 0, 1, or 2;
m is 0, 1, 2, 3, or 4;
n is 0, 1, 2, 3, or 4;
p is 0, 1, 2, 3, 4, or 5; and
wherein -L- is attached via a covalent bond to each of [symbol] and 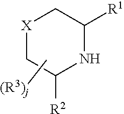
at a carbon, nitrogen, or oxygen atom, and each covalent bond independently replaces a hydrogen on the carbon, nitrogen, or oxygen atom.

3. The compound of claim 1, wherein -L- is

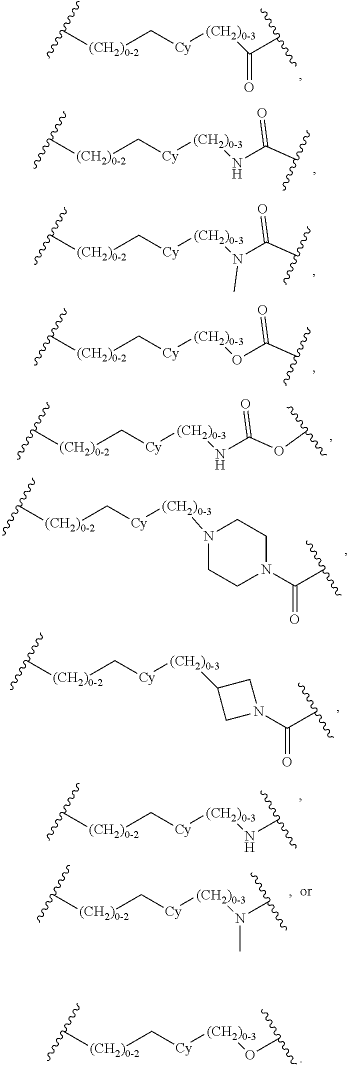

4-7. (canceled)

8. The compound of claim 1, wherein each of R¹ and R² is independently selected from phenyl, 5-6 membered monocyclic heteroaryl ring having 1-4 heteroatoms independently selected from nitrogen, oxygen, and sulfur, and an 8-10 membered partially aromatic or heteroaromatic bicyclic heterocyclic ring having 1-4 heteroatoms independently selected from nitrogen, oxygen, and sulfur; each of which is optionally substituted with m instances of R′.

9. (canceled)

10. The compound of claim 1, wherein each occurrence of R³ is independently C₁₋₇ aliphatic optionally substituted with 1-5 groups independently selected from halogen, —N(R)₂, and —OR, and wherein 1 or 2 methylene units of the aliphatic group are optionally replaced by —N(R)— or a methyl group is optionally replaced by —N(R)₂.

11. (canceled)

12. (canceled)

13. The compound of claim 1, wherein [symbol] is

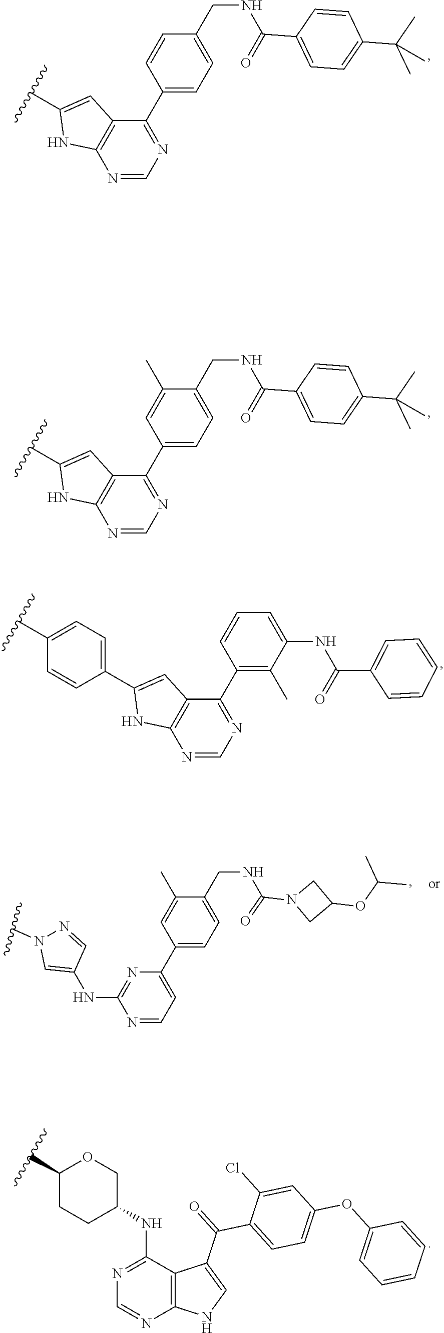 [SMILES: *c1cc2c(-c3ccc(CNC(=O)c4ccc(C(C)(C)C)cc4)cc3)ncnc2[nH]1 ; *c1cc2c(-c3ccc(CNC(=O)c4ccc(C(C)(C)C)cc4)c(C)c3)ncnc2[nH]1 ; *c1ccc(-c2cc3c(-c4cccc(NC(=O)c5ccccc5)c4C)ncnc3[nH]2)cc1 ; *n1cc(Nc2nccc(-c3ccc(CNC(=O)N4CC(OC(C)C)C4)c(C)c3)n2)cn1 ; *[C@@H]1CC[C@@H](Nc2ncnc3[nH]cc(C(=O)c4ccc(Oc5ccccc5)cc4Cl)c23)CO1]

14-38. (canceled)

39. The compound of claim 1, wherein the compound is of formula I-D-h, I-D-i, I-D-j, I-D-k, or I-B-l: 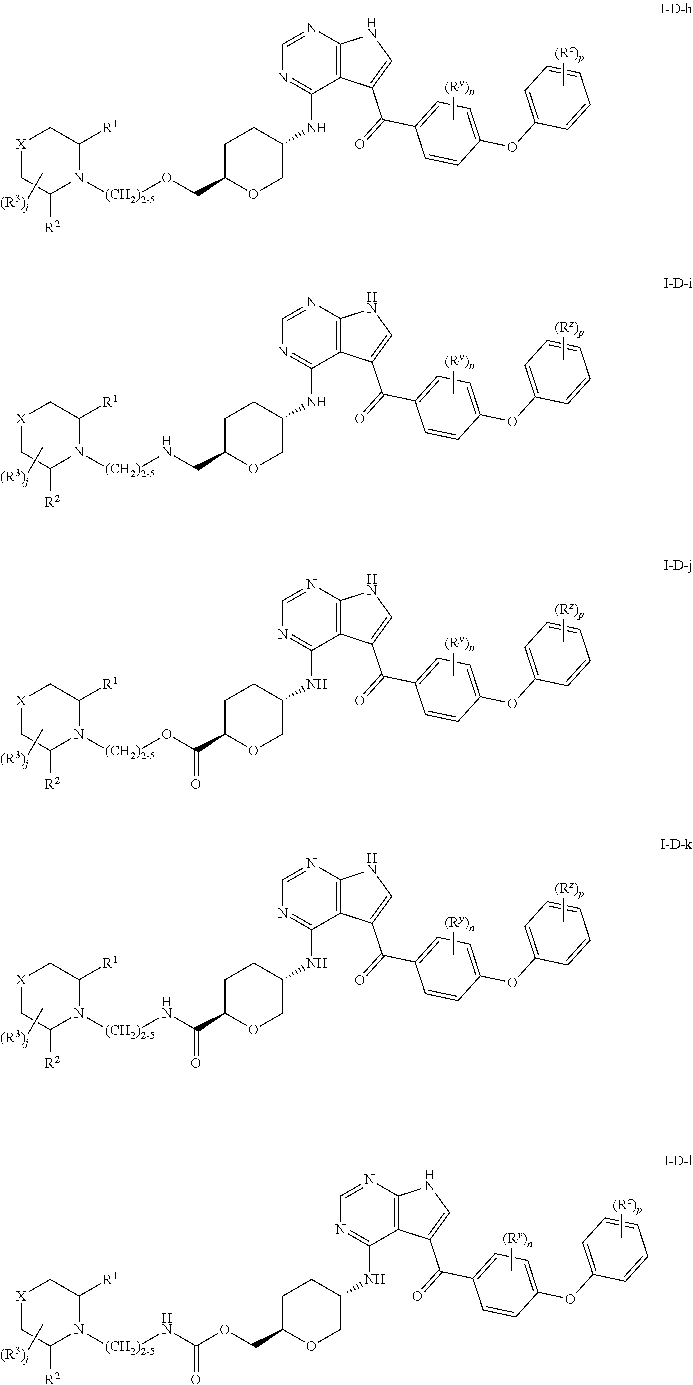
or a pharmaceutically acceptable salt thereof.

40. The compound of claim 1, wherein the compound is of formula I-D-m, I-D-n, I-D-o, I-D-p, or I-D-q: 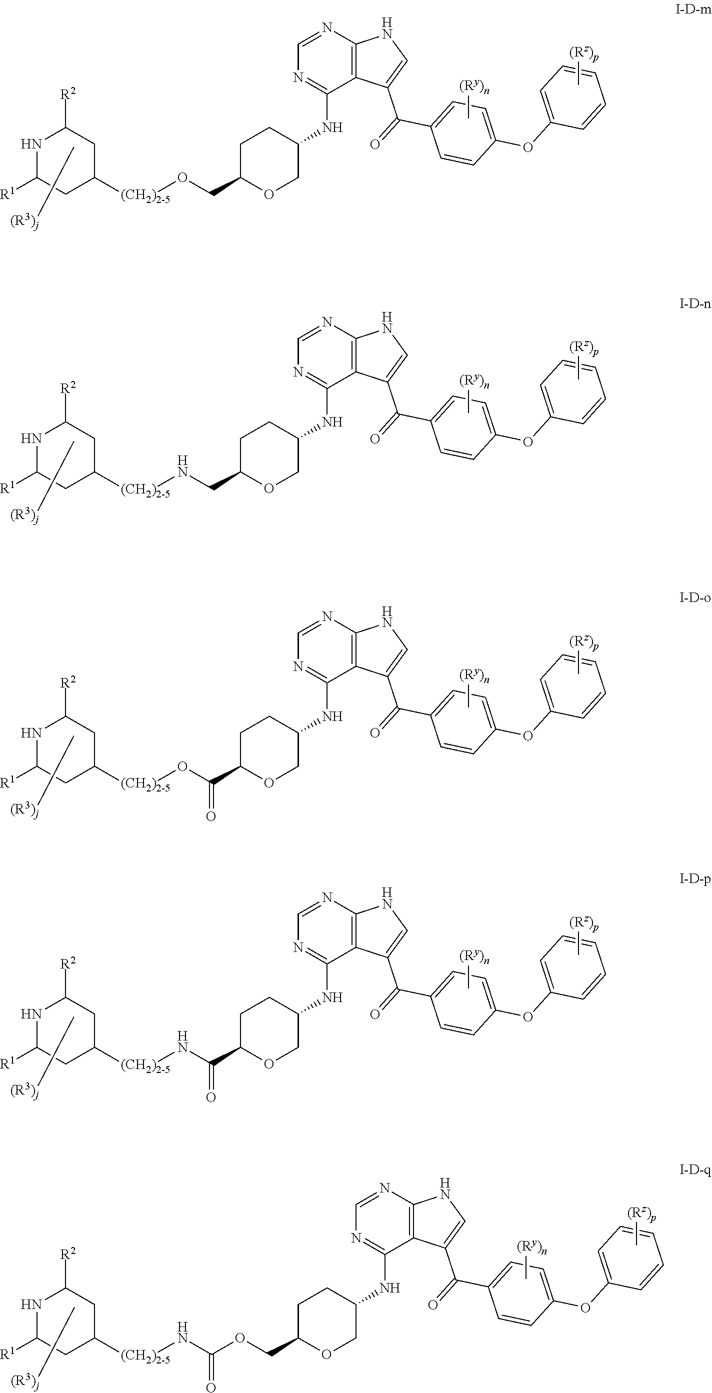
or a pharmaceutically acceptable salt thereof.

41. (canceled)

42. The compound of claim 1, wherein the compound is of formula I-D-r-a, I-D-s-a, I-D-t-a, I-D-u-a, or I-D-v-a: 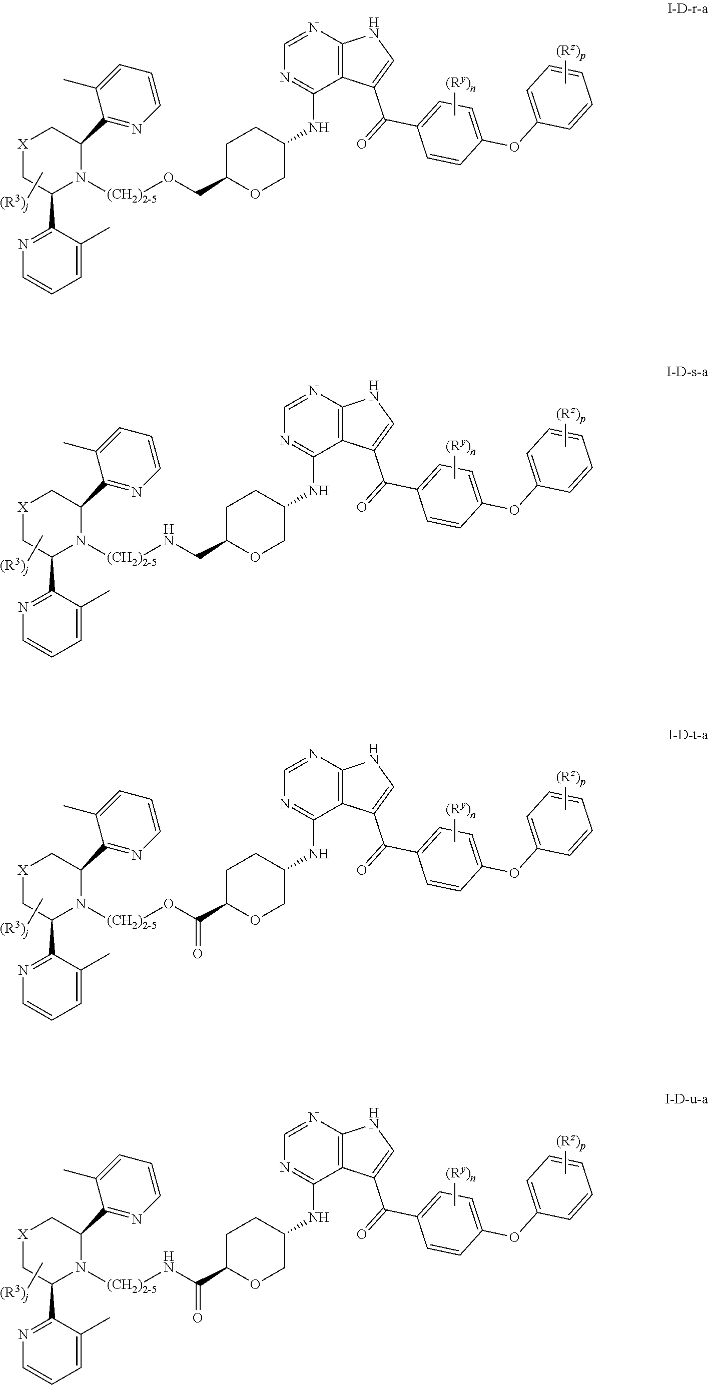 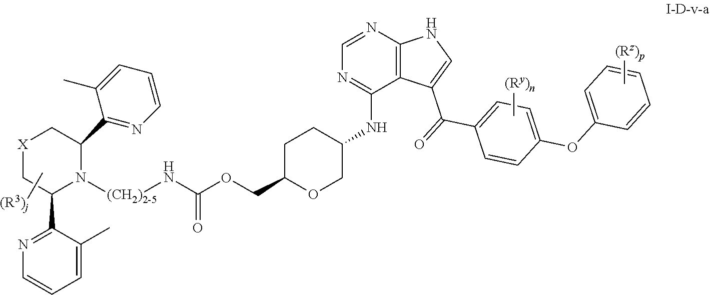
or a pharmaceutically acceptable salt thereof.

43. The compound of claim 1, wherein the compound is of formula I-D-r-b, I-D-s-b, I-D-t-b, I-D-u-b, or I-D-v-b: 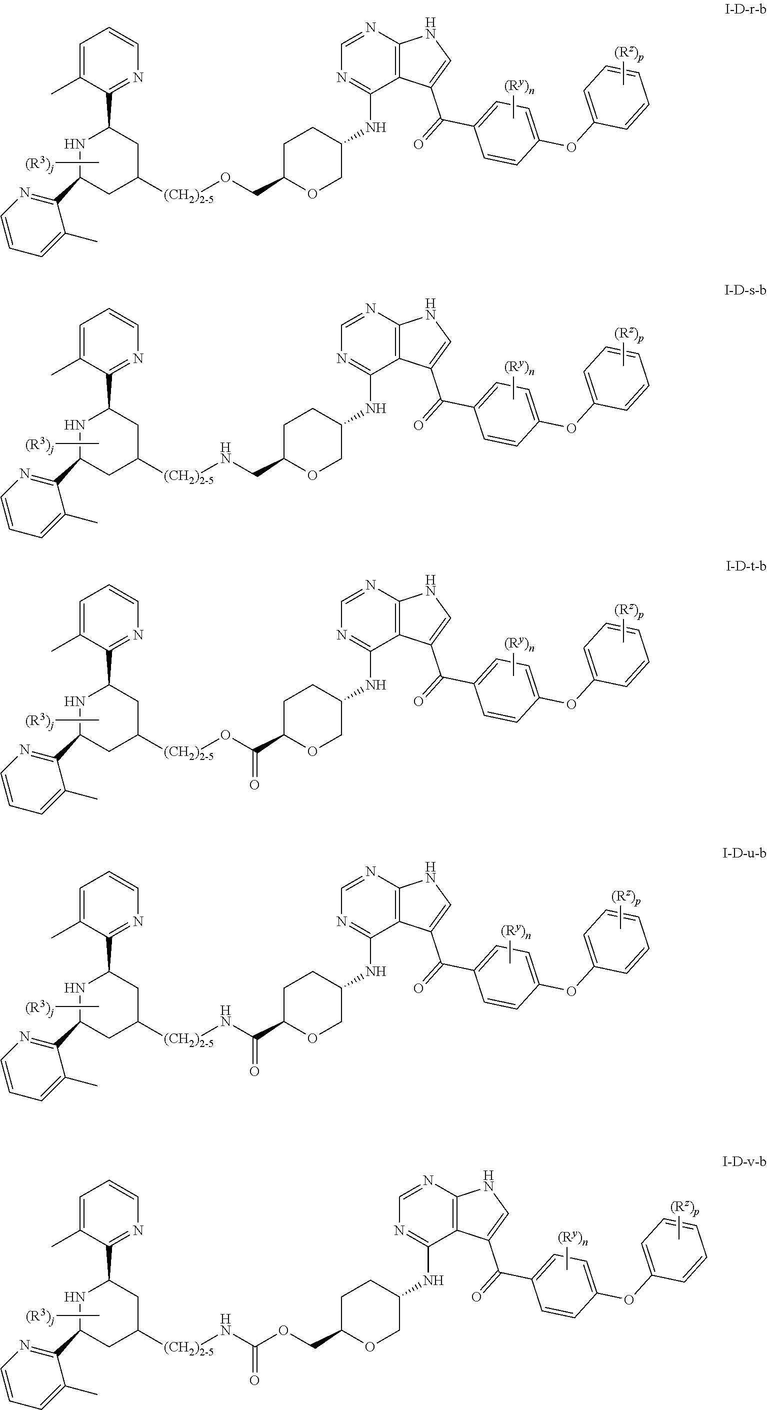
or a pharmaceutically acceptable salt thereof.

44. The compound of claim 1, wherein the compound is selected from those in Table 1, or a pharmaceutically acceptable salt thereof.

45. A pharmaceutical composition comprising a compound of claim 1, and a pharmaceutically acceptable excipient.

46. A method of treating a disease, disorder, or condition associated with CXCR4 and/or BTK, comprising administering to a subject in need thereof an effective amount of a compound of claim 1, or a pharmaceutically acceptable salt thereof.

47. The method of claim 46, wherein the disease, disorder, or condition is a B cell malignancy.

48. The method of claim 46, wherein the disease, disorder, or condition is selected from diffuse large B-cell lymphoma (DLBCL), follicular lymphoma, mantle-cell lymphoma, B-cell chronic lymphocytic leukemia (CLL), and Waldenstrom's macroglobulinemia.

49. The method of claim 46, wherein the disease, disorder, or condition is selected from inflammatory bowel disease, arthritis, systemic lupus erythematosus (SLE), vasculitis, idiopathic thrombocytopenia purpura (ITP), rheumatoid arthritis, psoriatic arthritis, osteoarthritis, Still's disease, juvenile arthritis, myasthenia gravis, Hashimoto's thyroiditis, Ord's thyroiditis, Graves' disease, autoimmune thyroiditis, Sjogren's syndrome, multiple sclerosis, Lyme neuroborreliosis, Guillain-Barre syndrome, acute disseminated encephalomyelitis, Addison's disease, opsoclonus-myoclonus syndrome, ankylosing spondylosis, antiphospholipid antibody syndrome, aplastic anemia, Fanconi Anemia, autoimmune hepatitis, autoimmune gastritis, pernicious anemia, celiac disease, Goodpasture's syndrome, optic neuritis, scleroderma, primary biliary cirrhosis, Reiter's syndrome, Takayasu's arteritis, temporal arteritis, warm autoimmune hemolytic anemia, Wegener's granulomatosis, psoriasis, alopecia universalis, Behcet's disease, chronic fatigue, dysautonomia, membranous glomerulonephropathy, endometriosis, interstitial cystitis, pemphigus vulgaris, bullous pemphigoid, neuromyotonia, scleroderma, vulvodynia, a hyperproliferative disease, rejection of transplanted organs or tissues, Acquired Immunodeficiency Syndrome (AIDS, caused by HIV), type 1 diabetes, graft versus host disease, transplantation, transfusion, anaphylaxis, allergies (e.g., allergies to plant pollens, latex, drugs, foods, insect poisons, animal hair, animal dander, dust mites, or cockroach calyx), type I hypersensitivity, allergic conjunctivitis, allergic rhinitis, and atopic dermatitis, asthma, appendicitis, atopic dermatitis, asthma, allergy, blepharitis, bronchiolitis, bronchitis, bursitis, cervicitis, cholangitis, cholecystitis, chronic graft rejection, colitis, conjunctivitis, Crohn's disease, cystitis, dacryoadenitis, dermatitis, dermatomyositis, encephalitis, endocarditis, endometritis, enteritis, enterocolitis, epicondylitis, epididymitis, fasciitis, fibrositis, gastritis, gastroenteritis, Henoch-Schonlein purpura, hepatitis, hidradenitis suppurativa, immunoglobulin A nephropathy, interstitial lung disease, laryngitis, mastitis, meningitis, myelitis myocarditis, myositis, nephritis, oophoritis, orchitis, osteitis, otitis, pancreatitis, parotitis, pericarditis, peritonitis, pharyngitis, pleuritis, phlebitis, pneumonitis, pneumonia, polymyositis, proctitis, prostatitis, pyelonephritis, rhinitis, salpingitis, sinusitis, stomatitis, synovitis, tendonitis, tonsillitis, ulcerative colitis, uveitis, vaginitis, vasculitis, or vulvitis, diffuse large B cell lymphoma, follicular lymphoma, chronic lymphocytic lymphoma, chronic lymphocytic leukemia, acute lymphocytic leukemia, B-cell prolymphocytic leukemia, lymphoplasmacytic lymphoma/Waldenstrom's macroglobulinemia, splenic marginal zone lymphoma, multiple myeloma (also known as plasma cell myeloma), non-Hodgkin's lymphoma, Hodgkin's lymphoma, plasmacytoma, extranodal marginal zone B cell lymphoma, nodal marginal zone B cell lymphoma, mantle cell lymphoma, mediastinal (thymic) large B cell lymphoma, intravascular large B cell lymphoma, primary effusion lymphoma, Burkitt lymphoma/leukemia, or lymphomatoid granulomatosis, breast cancer, prostate cancer, or cancer of the mast cells (e.g., astocyto a, mast cell leukemia, mast cell sarcoma, systemic mastocytosis), bone cancer, colorectal cancer, pancreatic cancer, diseases of the bone and joints including, without limitation, rheumatoid arthritis, seronegative spondyloarthropathies (including ankylosing spondylitis, psoriatic arthritis and Reiter's disease), Behcet's disease, Sjogren's syndrome, systemic sclerosis, osteoporosis, bone cancer, bone metastasis, a thromboembolic disorder, (e.g., myocardial infarct, angina pectoris, reocclusion after angioplasty, restenosis after angioplasty, reocclusion after aortocoronary bypass, restenosis after aortocoronary bypass, stroke, transitory ischemia, a peripheral arterial occlusive disorder, pulmonary embolism, deep venous thrombosis), inflammatory pelvic disease, urethritis, skin sunburn, sinusitis, pneumonitis, encephalitis, meningitis, myocarditis, nephritis, osteomyelitis, myositis, hepatitis, gastritis, enteritis, dermatitis, gingivitis, appendicitis, pancreatitis, cholocystitus, agammaglobulinemia, psoriasis, allergy, Crohn's disease, irritable bowel syndrome, ulcerative colitis, Sjogren's disease, tissue graft rejection, hyperacute rejection of transplanted organs, asthma, allergic rhinitis, chronic obstructive pulmonary disease (COPD), autoimmune polyglandular disease (also known as autoimmune polyglandular syndrome), autoimmune alopecia, pernicious anemia, glomerulonephritis, dermatomyositis, multiple sclerosis, scleroderma, vasculitis, autoimmune hemolytic and thrombocytopeniatates, Goodpasture's syndrome, atherosclerosis, Addison's disease, Parkinson's disease, Alzheimer's disease, diabetes, septic shock, systemic lupus erythematosus (SLE), rheumatoid arthritis, psoriatic arthritis, juvenile arthritis, osteoarthritis, chronic idiopathic thrombocytopenia purpura, Waldenstrom macroglobulinemia, atopic dermatitis, degenerative joint disease, vitiligo, autoimmune hypopituitarism, Behcet's disease, scleroderma, mycosis fungoides, acute inflammatory responses (such as acute respiratory distress syndrome and ischemia/reperfusion injury), and Graves' disease.

50. The method of claim 46, wherein the disease, disorder, or condition is mesothelioma, hepatobilliary (hepatic and billiary duct), bone cancer, pancreatic cancer, skin cancer, cancer of the head or neck, cutaneous or intraocular melanoma, ovarian cancer, colon cancer, rectal cancer, cancer of the anal region, stomach cancer, gastrointestinal (gastric, colorectal, and duodenal), uterine cancer, carcinoma of the fallopian tubes, carcinoma of the endometrium, carcinoma of the cervix, carcinoma of the vagina, carcinoma of the vulva, Hodgkin's Disease, cancer of the esophagus, cancer of the small intestine, cancer of the endocrine system, cancer of the thyroid gland, cancer of the parathyroid gland, cancer of the adrenal gland, sarcoma of soft tissue, cancer of the urethra, cancer of the penis, prostate cancer, testicular cancer, chronic or acute leukemia, chronic myeloid leukemia, lymphocytic lymphomas, cancer of the bladder, cancer of the kidney or ureter, renal cell carcinoma (RCC), carcinoma of the renal pelvis, non-Hodgkins's lymphoma, spinal axis tumors, brain stem glioma, pituitary adenoma, adrenocortical cancer, gall bladder cancer, multiple myeloma, cholangiocarcinoma, fibrosarcoma, neuroblastoma, retinoblastoma.

51. The method of claim 49, wherein the disease, disorder, or condition is Waldenstrom's macroglobulinemia.

52. The method of claim 46, wherein the disease, disorder, or condition is a primary immunodeficiency disease (PID).

53. The method of claim 52, wherein the PID is warts, hypogammaglobulinemia, infections, myelokathexis (WHIM) syndrome, severe congenital neutropenia (SCN), GATA2 deficiency (Mono MAC syndrome), idiopathic CD4+ T lymphocytopenia (ICL), Wiskott-Aldrich Syndrome (WAS), chronic idiopathic neutropenia (CIN), severe CIN, cyclic neutropenia, or Glycogen Storage Disease Ib.
